SEQUENCE LISTING (6 sequences)

SEQ ID NO: 1 - DNA, 1458 bp, Artificial sequence
  Codes for SEQ ID NO 2, a human 3A4 P450 protein truncated in its N-terminal region to delete the hydrophobic trans-membrane domain, and the region replaced by a short N-terminal sequence.
atggcatacggtactcattcacatggtctgtttaaaaaactgggaattccagggcccacacctctgccttttttgggaaatattttgtcctaccataagggcttttgtatgtttgacatggaatgtcataaaaagtatggaaaagtgtggggcttttatgatggtcaacagcctgtgctggctatcacagatcctgacatgatcaaaacagtgctagtgaaagaatgttattctgtcttcacaaaccggaggccttttggtccagtgggatttatgaaaagtgccatctctatagctgaggatgaagaatggaagagattacgatcattgctgtctccaaccttcaccagtggaaaactcaaggagatggtccctatcattgcccagtatggagatgtgttggtgagaaatctgaggcgggaagcagagacaggcaagcctgtcaccttgaaagacgtctttggggcctacagcatggatgtgatcactagcacatcatttggagtgaacatcgactctctcaacaatccacaagacccctttgtggaaaacaccaagaagcttttaagatttgattttttggatccattctttctctcaataacagtctttccattcctcatcccaattcttgaagtattaaatatctgtgtgtttccaagagaagttacaaattttttaagaaaatctgtaaaaaggatgaaagaaagtcgcctcgaagatacacaaaagcaccgagtggatttccttcagctgatgattgactctcagaattcaaaagaaactgagtcccacaaagctctgtccgatctggagctcgtggcccaatcaattatctttatttttgctggctatgaaaccacgagcagtgttctctccttcattatgtatgaactggccactcaccctgatgtccagcagaaactgcaggaggaaattgatgcagttttacccaataaggcaccacccacctatgatactgtgctacagatggagtatcttgacatggtggtgaatgaaacgctcagattattcccaattgctatgagacttgagagggtctgcaaaaaagatgttgagatcaatgggatgttcattcccaaaggggtggtggtgatgattccaagctatgctcttcaccgtgacccaaagtactggacagagcctgagaagttcctccctgaaagattcagcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacagatccccctgaaattaagcttaggaggacttcttcaaccagaaaaacccgttgttctaaaggttgagtcaagggatggcaccgtaagtggagcccaccatcaccattga

SEQ ID NO: 2 - protein, 485 aa, Artificial sequence
  Human 3A4 P450 protein truncated in its N-terminal region to delete the hydrophobic trans-membrane domain, and the region replaced by a short N-terminal sequence.
MAYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGAHHHH

SEQ ID NO: 3 - protein, 16 aa, Artificial sequence
  N-terminal sequence for expression of human 3A4 P450
MAYGTHSHGLFKKLGI

SEQ ID NO: 4 - DNA, 33 bp, Artificial sequence
  Primer
ggaattcatatggctctcatcccagacttggcc

SEQ ID NO: 5 - DNA, 47 bp, Artificial sequence
  Primer
tgcggtcgactcaatggtgatggtgggctccacttacggtgccatcc

SEQ ID NO: 6 - DNA, 69 bp, Artificial sequence
  Primer
ttaacatatggcatatggtactcattcacatggtctgtttaaaaaactgggaattccagggcccacacc

(19) United States
(12) Patent Application Publication
(10) Pub. No.: US 2005/0032119 A1
(43) Pub. Date: Feb. 10, 2005

(54) Crystal structure of cytochrome P450

(75) Inventors: Ian J. Tickle, Cambridge (GB); Clemens Vonrhein, Cambridge (GB); Dijana Matak Vinkovic, Cambridge (GB); Stewart Kirton, Cambridge (GB); Pamela A. Williams, Cambridge (GB); Harren Jhoti, Cambridge (GB)

Correspondence Address: NIXON & VANDERHYE, PC, 1100 N GLEBE ROAD, 8TH FLOOR, ARLINGTON, VA, 22201-4714, US

(73) Assignee: ASTEX TECHNOLOGY LTD., Cambridge (GB)

(21) Appl. No.: 10/833,296

(22) Filed: Apr. 28, 2004

Related U.S. Application Data
(63) Continuation-in-part of application No. 10/690,991, filed on Oct. 23, 2003.
Application No. 10/690,991 is a continuation-in-part of application No. PCT/GB02/02668, filed on May 30, 2002.
Application No. 10/690,991 is a continuation-in-part of application No. 10/221,036.
(60) Provisional application No. 60/479,448, filed on Jun. 19, 2003.
(60) Provisional application No. 60/421,063, filed on Oct. 25, 2002.

(30) Foreign Application Priority Data
Apr. 2, 2001 (GB) GB 0108214.8
Apr. 2, 2001 (GB) GB 0108212.2

(51) Int. Cl. G01N 33/53; G06F 19/00; G01N 33/48; G01N 33/50

(52) U.S. Cl. 435/7.1; 702/19

(57) ABSTRACT

The invention provides the crystal structure of the cytochrome P450 3A4 protein molecule. The structure is set out in Tables 1-4. The structure may be used in to model the interaction of compounds such as pharmaceuticals with this protein, and to determine the structure of related cytochrome P450 molecules.

The present application is a continuation-in-part of Ser. No. 10/690,991, which is a continuation-in-part of applications PCT/GB02/02668 filed May 30, 2002 and designating the US, and Ser. No. 10/221,036, filed Apr. 2, 2002, and claims benefit of the following U.S. Provisional Application Ser. Nos. 60/479,448, filed Jun. 19, 2003; 60/421,063, filed Oct. 25, 2002. U.S. Ser. No. 10/221,036 claims the benefit of priority of 60/306,873, filed Jul. 23, 2001, 60/306,874, filed Jul. 23, 2001, and UK applications GB 0108214.8 filed Apr. 2, 2001 and GB 0108212.2 filed Apr. 2, 2001. The contents of all these applications are incorporated herein by reference.

### FIELD OF THE INVENTION

The present invention relates to the human cytochrome P450 protein 3A4, methods for its crystallization, crystals and co-crystals of 3A4 and their 3-dimensional structures, and uses thereof.

### BACKGROUND TO THE INVENTION

Introduction to Cytochrome P450

Cytochrome P450s (CYP450) form a very large and complex gene superfamily of hemeproteins that metabolise physiologically important compounds in many species of microorganisms, plants and animals. Cytochrome P450s are important in the oxidative, peroxidative and reductive metabolism of numerous and diverse endogenous compounds such as steroids, bile, fatty acids, prostaglandins, leukotrienes, retinoids and lipids. Many of these enzymes also metabolise a wide range of xenobiotics including drugs, environmental compounds and pollutants. Their involvement in drug metabolism is extensive, it is estimated that 50% of all known drugs are affected in some way by the action of CYP450 enzymes. Significant resource is employed by the pharmaceutical industry to optimise drug candidates in order to avoid their detrimental interactions with the CYP450 enzymes. Another level of complication results from the fact that these enzymes exhibit different tissue distributions and polymorphisms between individuals and ethnic populations

Most mammalian P450s are located in the liver, but other organs and tissues have high concentrations of certain cytochrome P450s, including the intestinal wall, lung, kidney, adrenal cortex and nasal epithelium. Mammals have about 50 unique CYP450 genes and each family member is 45-55 KDa in size and contains a heme moiety that catalyses a two-electron activation of oxygen. The source of electrons may be used to classify CYP450s. Those that receive electrons in a three protein chain in which electrons flow from a flavin adenine dinucleotide (FAD) containing reductase, to an iron-sulphur protein, and then to P450 belong to the group of class I P450s, and include most of the bacterial enzymes. Class II P450s receive electrons from a reductase containing both FAD and flavin mononucleotide (FMN), and comprise the microsomal P450s that are the main culprits of drug metabolism. The mammalian microsomal cytochrome P450s are integral membrane proteins anchored by an N-terminal transmembrane spanning α-helix. They are inserted in the membrane of the endoplasmic reticulum by a short, highly hydrophobic N-terminal segment that acts as a non-cleavable signal sequence for insertion into the membrane. The remainder of the mammalian cytochrome P450 protein is a globular structure that protrudes into the cytoplasmic space. Hence, the bulk of the enzyme faces the cytoplasmic surface of the lipid bilayer. P450s require other membranous enzymatic components for activity including the flavoprotein NADPH-cytochrome P450 oxidoreductase and, in some cases, cytochrome b5. A single cytochrome P450 oxidoreductase supports the activity of all the mammalian microsomal enzymes by interacting directly with the P450s and transferring the required two electrons from NADPH. Cytochrome P450s are able to incorporate one of the two oxygen atoms of an O₂ molecule into a broad variety of substrates with concomitant reduction of the other oxygen atom by two electrons to H₂O. Cytochrome P450 are known to catalyse hydroxylations, epoxidation, N-, S-, and O-dealkylations, N-oxidations, sulfoxidations, dehalogenations, and other reactions.

The genes of the P450 superfamily have been categorized by Nelson et al (Pharmacogenetics, 6; 1-42, 1996) who proposed a systematic nomenclature for the family members. This nomenclature is used widely in the art, and is adopted herein. Nelson et al provide cross-references to sequence database entries for P450 sequences.

Homo sapiens has 17 cytochrome P450 gene families and 42 subfamilies that total more than 50 sequenced isoforms. Cytochrome P450s from families 1, 2 and 3 constitute the major pathways for drug metabolism. Many drugs rely on hepatic metabolism by cytochrome P450s for clearance from the circulation and for pharmacological inactivation. Conversely, some drugs have to be converted in the body to their pharmacologically active metabolites by P450s. Many promising lead compounds are terminated in the development phase due to their interaction with one or more P450s. One of the greatest problems in drug discovery is the prediction of the role of cytochrome P450s on the metabolism or modification of drug leads. Early detection of metabolic problems associated with a chemical lead series is of paramount importance for the pharmaceutical industry. Obtaining crystal structures of the main human drug metabolising cytochrome P450s would be highly valuable for drug design, as this would provide detailed information on how P450 enzymes recognize drug molecules and the mode of drug binding. This in turn would allow drug companies to develop strategies to modify metabolic clearance and decrease the attrition rates of compounds in development.

The major human CYP450 isoforms involved in drug metabolism are CYP1A2, CYP2C9, CYP2C19, CYP2D6 and CYP3A4. The level of sequence identity between these family members ranges from about 20-80%, with much of the variability within the residues involved in substrate recognition. CYP450 enzymes are also present in bacteria and much of the understanding of substrate recognition is derived from crystal structures obtained of bacterial CYP450 enzymes.

CYP3A is both the most abundant and most clinically significant subfamily of cytochrome P450 enzymes. The CYP3A subfamily has four human isoforms, 3A4, 3A5, 3A7 and 3A43, CYP3A4 being the most commonly associated with drug interactions. The CYP3A isoforms make up approximately 50% of the liver's total cytochrome P450 and are widely expressed throughout the gastrointestinal tract, kidneys and lungs and therefore are ultimately responsible for the majority of first-pass metabolism. This is important as increases or decreases in first-pass metabolism can have the effect of administering a much smaller or larger dose of drug than usual. More than 150 drugs are known substrates of CYP3A4, including many of the opiate analgesics, steroids, antiarrhythmic agents, tricyclic antidepressants, calcium-channel blockers and macrolide antibiotics. Although several substrates show age-dependent reductions in elimination, the enzyme itself does not appear to be altered. CYP3A4 is important in the metabolism of many drugs including cyclosporine, codeine, tamoxifen, lovastatin, and many more, and endogenous compounds such as testosterone, estradiol and cortisol. Ketoconazole, itraconazole, erythromycin, clarithromycin, diltiazem, fluvoxamine, nefazodone, and dihydroxybergamottin and various substances found in grapefruit juice, green tea and other foods are potent inhibitors of CYP3A4 and are known to be responsible for many drug interactions. These interactions can have serious clinical consequences.

Background to Crystallisation

It is well-known in the art of protein chemistry, that crystallising a protein is a chancy and difficult process without any clear expectation of success. It is now evident that protein crystallization is the main hurdle in protein structure determination. For this reason, protein crystallization has become a research subject in and of itself, and is not simply an extension of the protein crystallographer's laboratory. There are many references which describe the difficulties associated with growing protein crystals. For example, Kierzek, A. M. and Zielenkiewicz, P., (2001), Biophysical Chemistry, 91, 1-20, Models of protein crystal growth, and Vviencek, J. M. (1999) Annu. Rev. Biomed. Eng., 1, 505-534, New Strategies for crystal growth.

It is commonly held that crystallization of protein molecules from solution is the major obstacle in the process of determining protein structures. The reasons for this are many; proteins are complex molecules, and the delicate balance involving specific and non-specific interactions with other protein molecules and small molecules in solution, is difficult to predict.

Each protein crystallizes under a unique set of conditions, which cannot be predicted in advance. Simply supersaturating the protein to bring it out of solution may not work, the result would, in most cases, be an amorphous precipitate. Many precipitating agents are used, common ones are different salts, and polyethylene glycols, but others are known. In addition, additives such as metals and detergents can be added to modulate the behaviour of the protein in solution. Many kits are available (e.g. from Hampton Research), which attempt to cover as many parameters in crystallization space as possible, but in many cases these are just a starting point to optimise crystalline precipitates and crystals which are unsuitable for diffraction analysis. Successful crystallization is aided by a knowledge of the proteins behaviour in terms of solubility, dependence on metal ions for correct folding or activity, interactions with other molecules and any other information that is available. Even so, crystallization of proteins is often regarded as a time-consuming process, whereby subsequent experiments build on observations of past trials.

In cases where protein crystals are obtained, these are not necessarily always suitable for diffraction analysis; they may be limited in resolution, and it may subsequently be difficult to improve them to the point at which they will diffract to the resolution required for analysis. Limited resolution in a crystal can be due to several things. It may be due to intrinsic mobility of the protein within the crystal, which can be difficult to overcome, even with other crystal forms. It may be due to high solvent content within the crystal, which consequently results in weak scattering. Alternatively, it could be due to defects within the crystal lattice which mean that the diffracted x-rays will not be completely in phase from unit to unit within the lattice. Any one of these or a combination of these could mean that the crystals are not suitable for structure determination.

Some proteins never crystallize, and after a reasonable attempt it is necessary to examine the protein itself and consider whether it is possible to make individual domains, different N or C-terminal truncations, or point mutations. It is often hard to predict how a protein could be re-engineered in such a manner as to improve crystallisability. Our understanding of crystallisation mechanisms are still incomplete and the factors of protein structure which are involved in crystallisation are poorly understood.

Determination of Protein Structure.

A mathematical operation termed a Fourier transform relates the diffraction pattern observed from a crystal and the molecular structure of the protein comprising the crystal. A Fourier transform may be considered to be a summation of sine and cosine waves each with a defined amplitude and phase. Thus, in theory, it is possible to calculate the electron density associated with a protein structure by carrying out an inverse Fourier transform on the diffraction data. This, however, requires amplitude and phase information to be extracted from the diffraction data. Amplitude information may be obtained by analysing the intensities of the spots within a diffraction pattern. Current technologies for generating x-rays and recording diffraction data lead to loss of all phase information. This “phase information” must be in some way recovered and the loss of this information represents the “crystallographic phase problem”. The phase information necessary for carrying out the inverse Fourier transform can be obtained via a variety of methods. If a protein structure exists a set of theoretical amplitudes and phases may be calculated using the protein model and then the theoretical phases combined with the experimentally derived amplitudes. An electron density map may then be calculated and the protein structure observed.

If there is no known structure of the protein then alternative methods for obtaining phases must be explored. One method is multiple isomorphous replacement (MIR). This relies on soaking “heavy atom” (i.e. platinum, uranium, mercury, etc) compounds into the crystals and observing how their incorporation into the crystals modifies the spot intensities observed in the diffraction pattern. This method relies on the heavy atoms being incorporated into the protein at a finite number of defined sites. It is a pre-requisite of an isomorphous replacement experiment that the heavy atom soaked crystals remain isomorphous. That is, there should be no appreciable alterations in the physical characteristics of the protein crystal (i.e. perturbations to crystallographic cell dimensions, or significant loss of resolution). Perturbations to the physical properties of the crystal are termed non-isomorphisms and prevent this type of experiment being successfully completed. Successful isomorphous incorporation of heavy atoms into a protein crystal results in the intensities of the spots within the diffraction pattern obtained from the crystal being modified, as compared to the data collected from an identical, unsoaked, (native) crystal. The diffraction data obtained from a successful isomorphous replacement experiment are termed a “derivative” dataset. By mathematically analysing the “native” and “derivative” datasets it is possible to extract preliminary phase information from the datasets. This phase information, when combined with the experimentally obtained amplitudes from the native dataset, enables an electron density map of the unknown protein molecule to be calculated using the Fourier transform method.

An alternative method for obtaining phase information for a protein of unknown structure is to perform a multi-wavelength anomalous dispersion (MAD) experiment. This relies on the absorption of X-rays by electrons at certain characteristic X-ray wavelengths. Different elements have different characteristic absorption edges. Anomalous scattering by atoms within a protein will modify the diffraction pattern obtained from the protein crystal. Thus if a protein contains atoms which are capable of anomalous scattering a diffraction dataset (anomalous dataset) may be collected at an X-ray wavelength at which this anomalous scattering is maximal. By altering the X-ray wavelength to a value at which there is no anomalous scattering a native dataset may then be collected. Similarly to the MIR case, by mathematically processing the anomalous and native datasets the phase information necessary for the calculation of an electron density map may be determined. The most usual way to introduce anomalous scatterers into a protein is to replace the sulphur containing methionine amino acid residues with selenium containing seleno-methionine residues. This is done by generating recombinant protein that is isolated from cells grown on growth media that contain seleno-methionine. Selenium is capable of anomalously scattering X-rays and may thus be used for a MAD experiment. Further methods for phase determination such as single isomorphous replacement (SIR), single isomorphous replacement anomalous scattering (SIRAS) and direct methods exist, but the principles behind them are similar to MIR and MAD.

The final method generally available for the calculation of the phases necessary for the determination of an unknown protein structure is molecular replacement. This method relies upon the assumption that proteins with similar amino acid sequences (primary sequences) will have a similar fold and three-dimensional structure (tertiary structure). Proteins related by amino acid sequence are termed homologous proteins. If an X-ray diffraction dataset has been collected from a crystal whose protein structure is not known, but a structure has been determined for a homologous protein, then molecular replacement can be attempted. Molecular replacement is a mathematical process that attempts to correlate the dataset obtained from a new protein crystal with the theoretical diffraction pattem calculated for a protein of known structure. If the correlation is sufficiently high some phase information can be extracted from the known protein structure and combined with the amplitudes obtained from the new protein dataset. This enables calculation of a preliminary electron density map for the protein of unknown structure.

If an electron density map has been calculated for a protein of unknown structure then the amino acids comprising the protein must be fitted into the electron density for the protein. This is normally done manually, although high resolution data may enable automatic model building. The process of model building and fitting the amino acids to the electron density can be both a time consuming and laborious process. Once the amino acids have been fitted to the electron density it is necessary to refine the structure. Refinement attempts to maximise the correlation between the experimentally calculated electron density and the electron density calculated from the protein model built. Refinement also attempts to optimise the geometry and disposition of the atoms and amino acids within the user-constructed model of the protein structure. Sometimes manual re-building of the structure will be required to release the structure from local energetic minima. There are now several software packages available that enable an experimentalist to carry out refinement of a protein structure. There are certain geometry and correlation diagnostics that are used to monitor the progress of a refinement. These diagnostic parameters are monitored and rebuilding/refinement continued until the experimenter is satisfied that the structure has been adequately refined.

Description of Anomalous Scattering Theory

If the energy of incident X-rays is close to the minimum energy that is required to eject a bound electron from an innermost shell of an atom, the scattering of the X-rays is described as “anomalous”. In the process of “normal” scattering, the electrons are forced to undergo vibrations at the same frequency as that of the incident X-ray photon, emitting elastically scattered photons (i.e. no change in frequency) in the process. However, because this frequency is far from the natural frequency of vibration of the electron there is no effect on the scattered photon from this natural vibration. In the process of “anomalous” scattering, the frequency of the incident photon is close to the natural frequency of the electron, resulting in a resonance effect, which is manifested as a dispersion (decrease in velocity, though still no change in frequency) of the photon, as well as a vibration damping effect, which is manifested as absorption (decrease in intensity) of a fraction of the incident photons.

The anomalously scattered photon will thus have a phase angle associated with it that is retarded when compared with one being scattered normally, all other conditions being equal. If the structure consists of a mixture normal and anomalous scatterers this phase lag results in the breakdown of Friedel's law, as pairs of reflections with indices (h,k,l) and (−h,−k,−l) that are diffracted from opposite sides of the same crystal plane no longer have the same amplitudes.

By careful measurement of the two reflection intensities, and by consideration of their relative amplitudes, it is possible to make an initial estimate of the phases of all reflections that have been observed.

In theory all atoms could give rise to an anomalous scattering effect if irradiated with X-ray radiation of the appropriate wavelength. However as the scattering is directly proportional to the weight of the scatterer, heavier elements are normally chosen, e.g. sulphur or larger. The choice of element is also dependent on the ability to tune the energy of the X-rays to the required transition energy. As access to tuneable synchrotron X-radiation has become routine, the MAD technique has come of age. Incorporation of an anomalous scatterer may be via a number of routes e.g. by soaking crystals in solutions containing heavy atoms which then bind to the protein, by expressing recombinant proteins in media in which an element has been replaced by a suitable heavier element (e.g. the replacement of methionine with selenomethionine) leading to the incorporation of the element in certain amino acids themselves, or making use of naturally occurring co-factors which contain heavy elements.

As the contribution from the anomalous scatterer may be small, it is often important to obtain well-recorded, redundant data, and to facilitate detection of what may be a small signal, it is helpful to have a reference dataset to which the anomalous dataset can be compared. The routine collection of X-ray data at cryo-temperatures has prolonged crystal lifetime and has made collection of multiple datasets (at different wavelengths) from a single crystal now feasible for many crystal systems. Collection and analysis of multiple datasets from a single crystal has the advantage of eliminating all effects related to non-isomorphism (variations in structure between different crystals due to random variations in soaking and/or freezing conditions).

In the case of cytochrome P450, the haem group that forms the site of enzymatic activity naturally contains a single iron atom. Iron has transition energies at the high energies (long wavelengths) obtainable at tunable synchrotron beamlines.

P450 Crystal Stuctures.

As of 2002, eight cytochrome P450 structures had been solved by X-ray crystallography and were available in the public domain. Six structures correspond to bacterial cytochrome P450s: P450cam (CYP101 Poulos et al., 1985, J. Biol. Chem., 260, 16122), the hemeprotein domain of P450BM3 (CYP102, Ravichandran etal., 1993, Science, 261, 731), P450terp (CYP108, Hasemann et al., 1994, J. Mol. Biol. 236, 1169), P450eryF (CYP107A1, Cupp-Vickery and Poulos, 1995, Nature Struct. Biol. 2, 144), P450 14α-sterol demethylase (CYP51, Podust et al., 2001, Proc. Natl. Acad. Sci. USA, 98, 3068) and the crystal structure of a thermophilic cytochrome P450 (CYP119) from Archaeon sulfolobus solfataricus was solved (Yano et al., 2000, J. Biol. Chem. 275, 31086). The structure of cytochrome P450nor was obtained from the denitrifying fungus Fusarium oxysporum (Shimizu et al. 2000, J. Inorg. Biochem. 81, 191). The eighth structure is that of the rabbit 2C5 isoform, the first structure of a mammalian cytochrome P450 (Williams etal. 2000, Mol. Cell. 5, 121).

WO 03/035693 describes the crystallisation of a human 2C9 P450 protein molecule and provides an analysis of the protein crystal structure.

Our understanding of the structural variability of these enzymes has been advanced further in recent years, with the addition of nine non-mammalian crystal structures; CYP152A1 from Bacillus subtilis (Lee et al, 2003, J. Biol. Chem, 278, 9761), CYP165B1 from Amycolatopsis orientalis (P450 OxyB) (Zerbe et al, 2002, J. Biol. Chem, 277, 47476), CYP165C1 from Amycolatopsis orientalis (P450 OxyC) (Pylypenko et al, 2002, J. Biol. Chem, 278, 46727), CYP167A1 from Polyangium cellulosum (P450 EpoK) (Nagano et al, 2003, J Biol. Chem. 278, 44886), CYP119A2 from sulfolobus tokodaii (CYP119) (Yano et al 2000, J. Biol. Chem. 31086), CYP175A1 from Thermus thermophilus strain HB27 (Yano et al, 2003, J. Biol. Chem. 278, 608), CYP121 from mycobacterium tuberculosis (Leys et al, 2003, J. Biol. Chem. 278, 5141), CYP154C1 from streptomyces coelicolor (Podust et al, 2003, J. Biol. Chem. 278, 12214), and CYP154A1 from streptomyces coelicolor (Podust et al, 2004, Protein Sci., 13, 255).

In addition, another two mammalian structures have been solved, namely the rabbit CYP2B4 in the absence (Scott et al, 2003, P.N.A.S., 100, 13196) and presence of compound (Scott et al, 2004, J. Biol. Chem, April 2004; 10.1074/jbc.M403349200), and the human CYP2C8 (Schoch et al, 2003, Biochemistry, 279, 9497) in the absence of compound. Two compound complexes with rabbit CYP2C5 with diclofenac and a sulfaphenazole derivative have been also been solved (Wester et al, 2003, Biochemistry, 42, 9335;Wester et al, 2003, Biochemistry, 42, 6370).

The reason why the mammalian cytochrome P450s have been particularly difficult to crystallize, compared to their bacterial counterparts, resides in the nature of these proteins. The bacterial cytochrome P450s are soluble whereas the mammalian P450s are membrane-associated proteins. Thus, structural studies on mammalian cytochrome P450s may use the combination of heterologous expression systems that allow expression of single cytochrome P450s at high concentration with modification of their sequences to improve the solubility and the behaviour of these proteins in solution.

Due to significant sequence differences from both the bacterial proteins and rabbit proteins, to fully understand the role of the human CYP450 enzymes in drug metabolism, the crystal structures of other human isoforms are still required.

### DISCLOSURE OF THE INVENTION

The present invention relates to the crystal structure of human 3A4, which allows the binding location of the substrates in the enzyme to be investigated and determined.

More particularly, the present inventors have obtained apo crystals of 3A4 in two different space group forms, and co-crystals of 3A4. Thus in one aspect, the invention provides a three dimensional structure of 3A4 set out in any one of Tables 1-4, and uses thereof.

In general aspects, the present invention is concerned with the provision of a 3A4 structure and its use in modelling the interaction of molecular structures, e.g. potential and existing pharmaceutical compounds, prodrugs, P450 inhibitors or substrates, or fragments of such compounds, prodrugs, inhibitors or substrates with this 3A4 structure.

These and other aspects and embodiments of the present invention are discussed below.

The above aspects of the invention, both singly and in combination, all contribute to features of the invention, which are advantageous.

### BRIEF DESCRIPTION OF THE TABLES

Table 1 (FIG. 1) sets out the coordinate data of the structure of 3A4.

Table 2 (FIG. 2) sets out the coordinate data of a co-complex of 3A4 and metyrapone.

Table 3 (FIG. 3) sets out the coordinate data of a co-complex of 3A4 and progesterone.

Table 4 (FIG. 4) sets out the coordinate data of the structure of an alternate crystal form of 3A4

Table 5 (FIG. 5) sets out one possible set of coordinate data of a loop region of 3A4.

Table 6 details binding site residues of 3A4.

Table 7 sets out newly identified binding site residues of 3A4.

### BRIEF DESCRIPTION OF THE DRAWINGS

FIG. 1 sets out Table 1.

FIG. 2 sets out Table 2.

FIG. 3 sets out Table 3.

FIG. 4 sets out Table 4.

FIG. 5 sets out Table 5

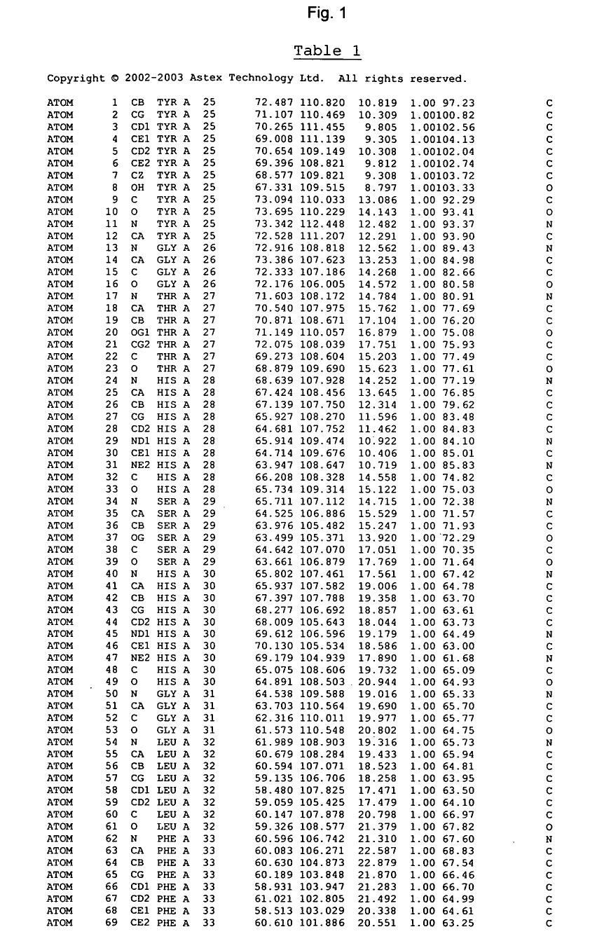

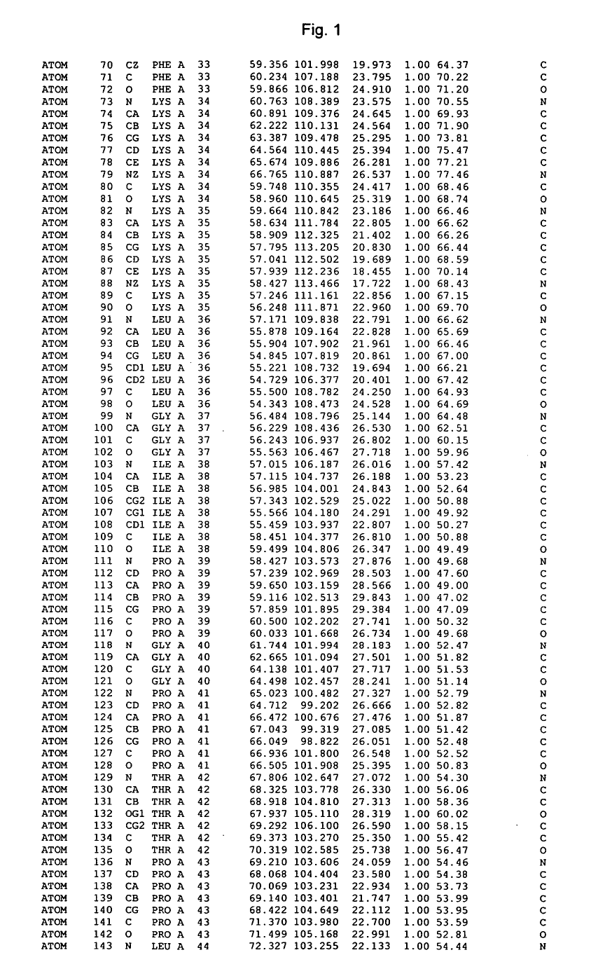

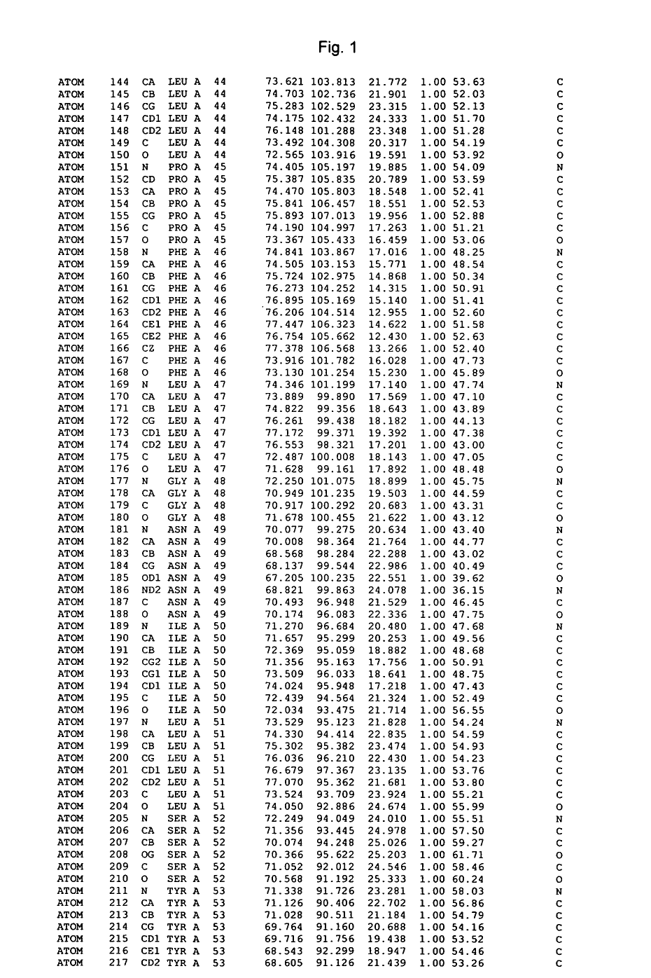

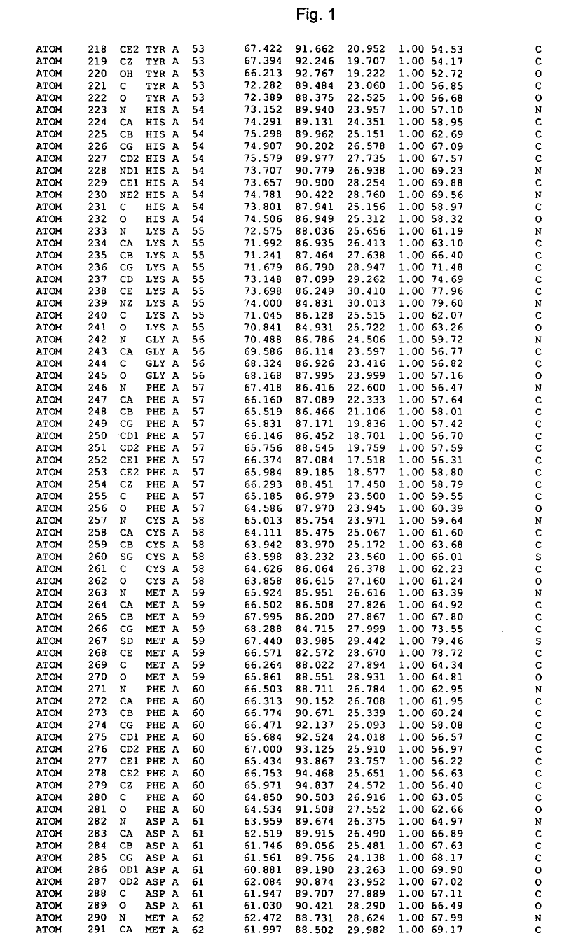

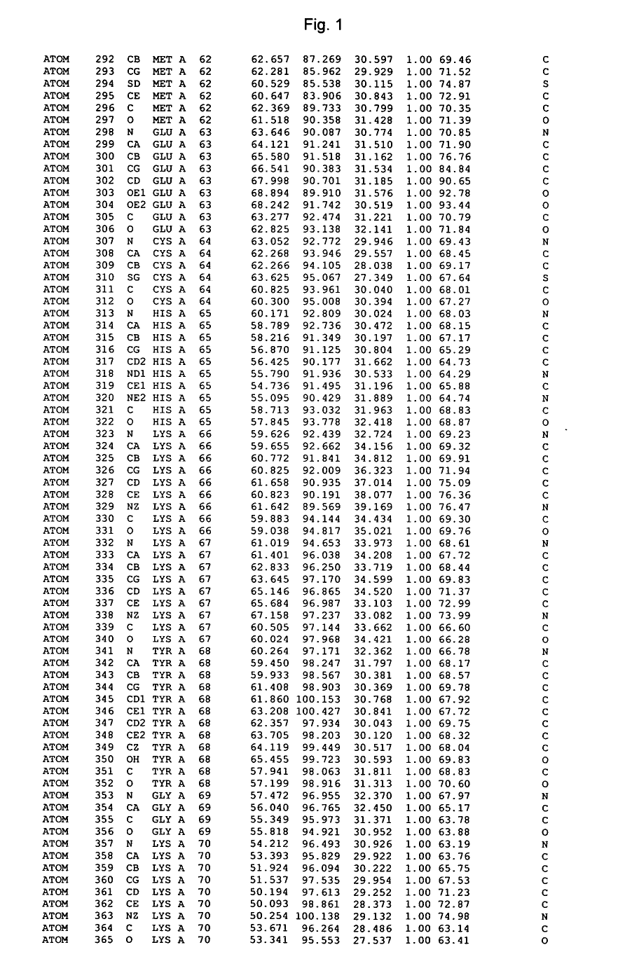

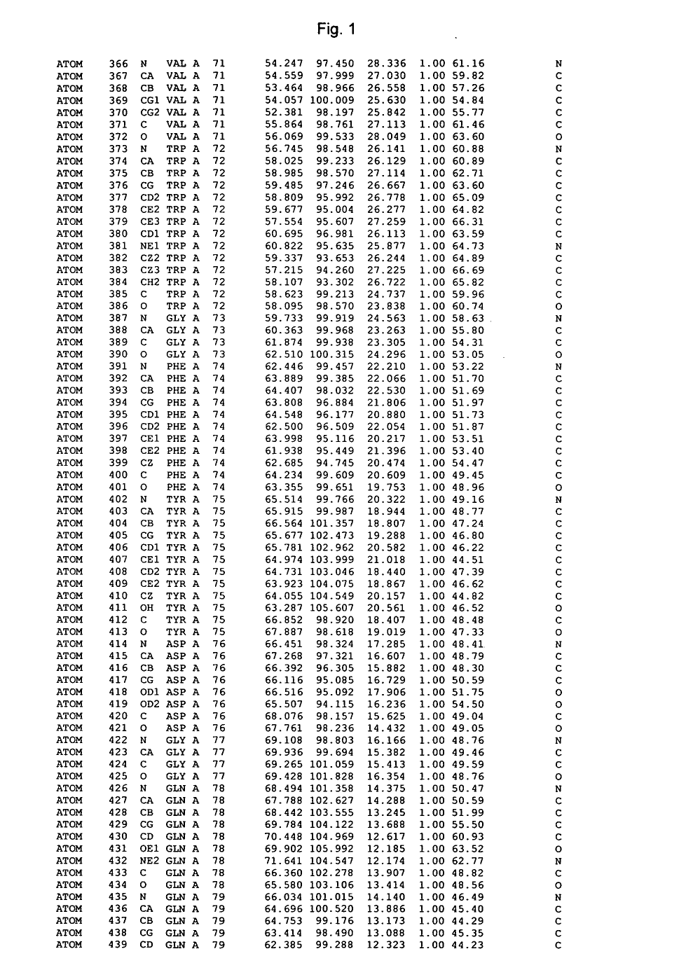

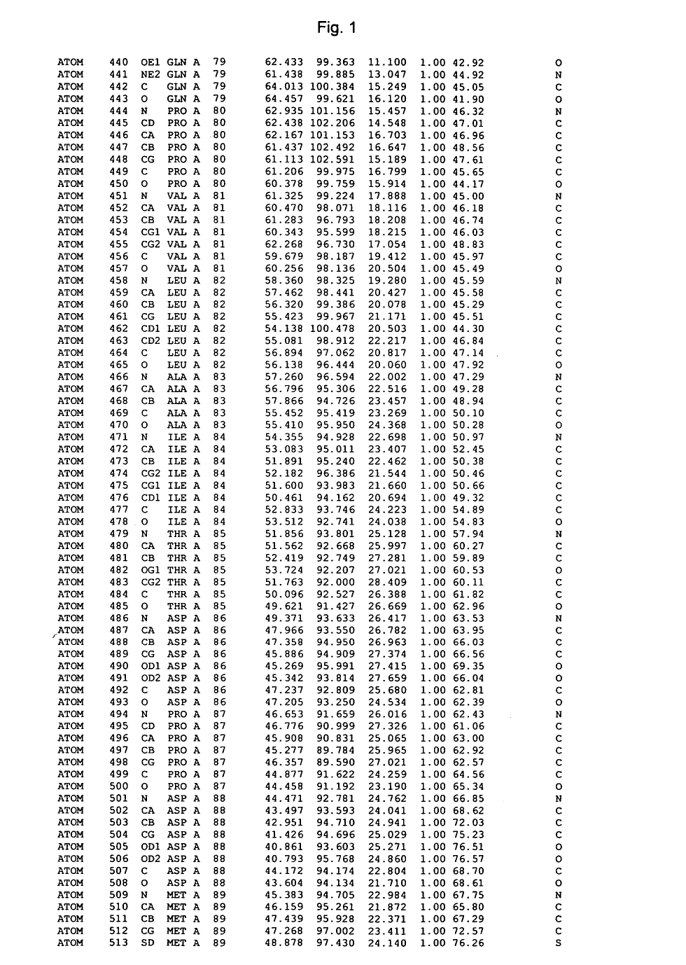

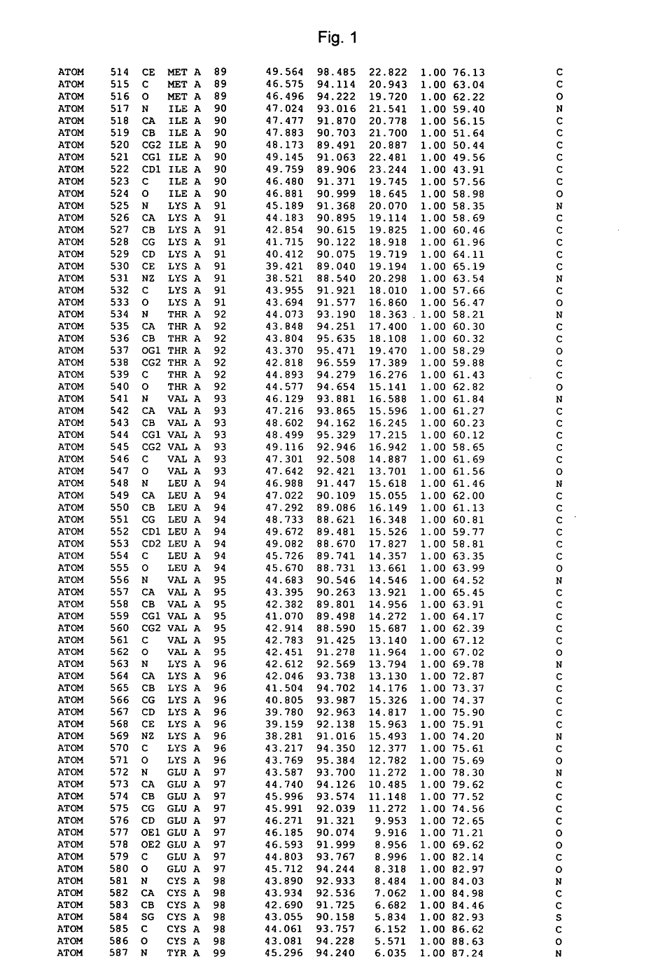

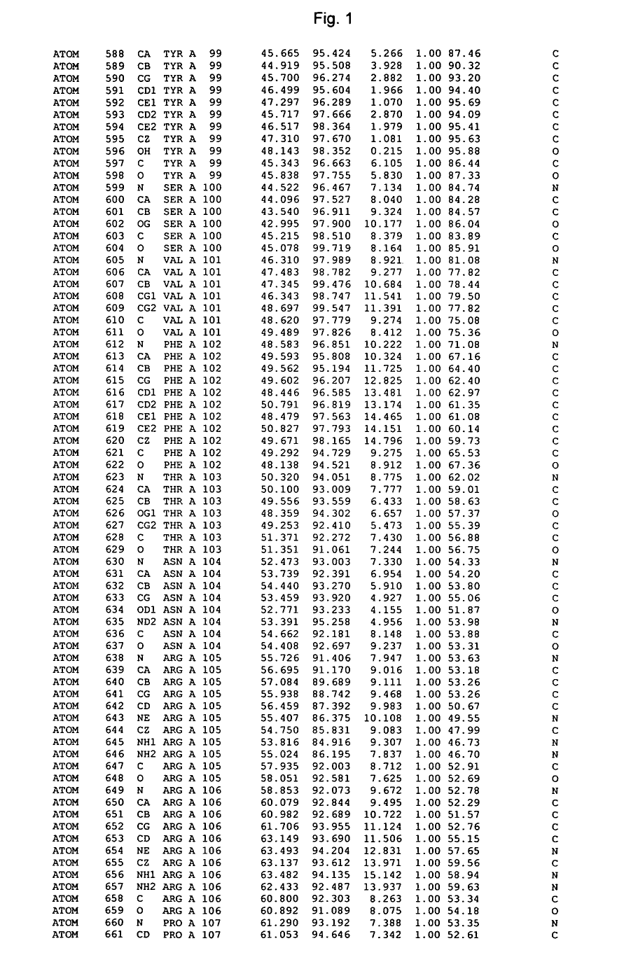

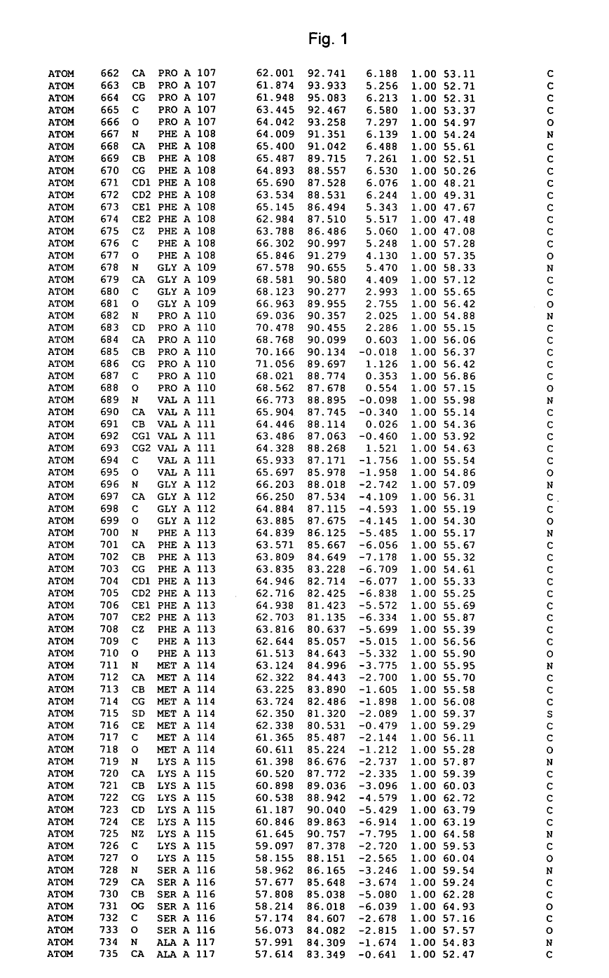

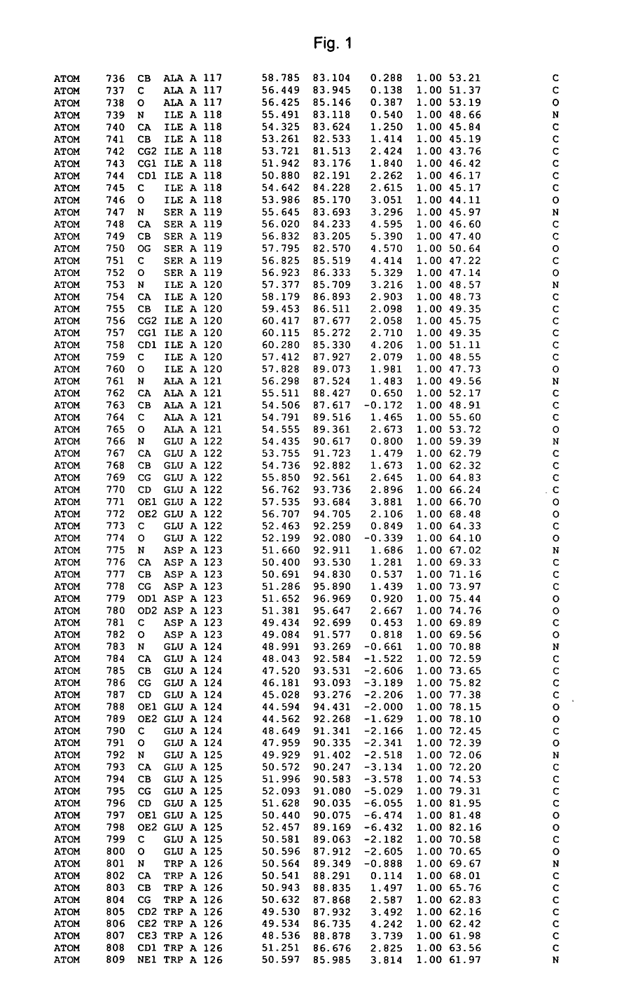

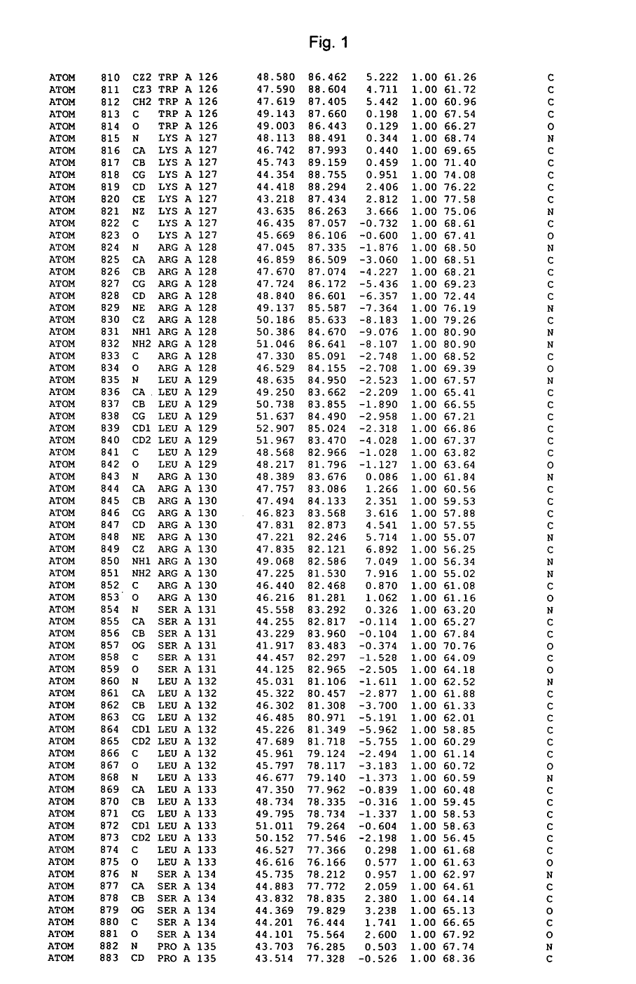

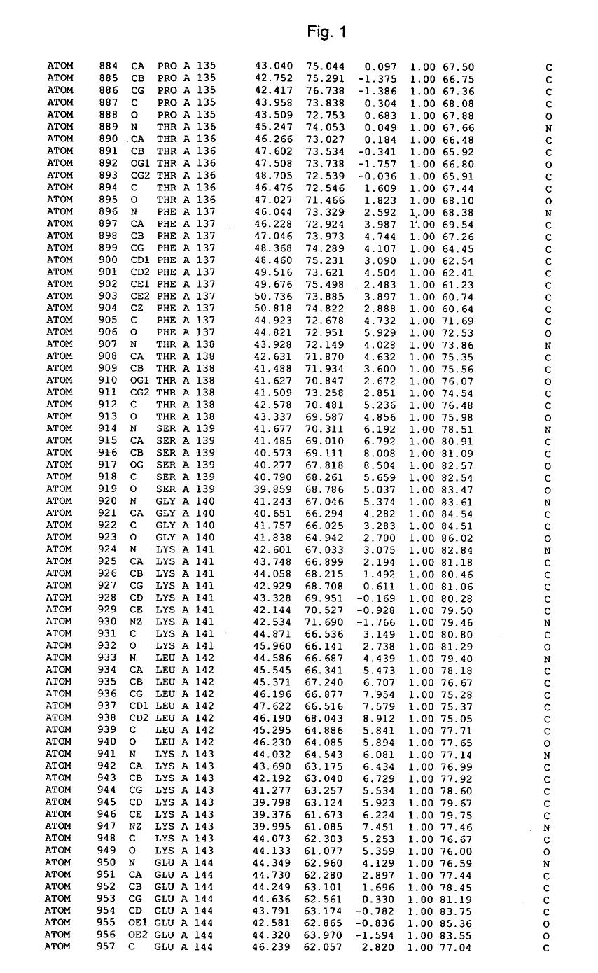

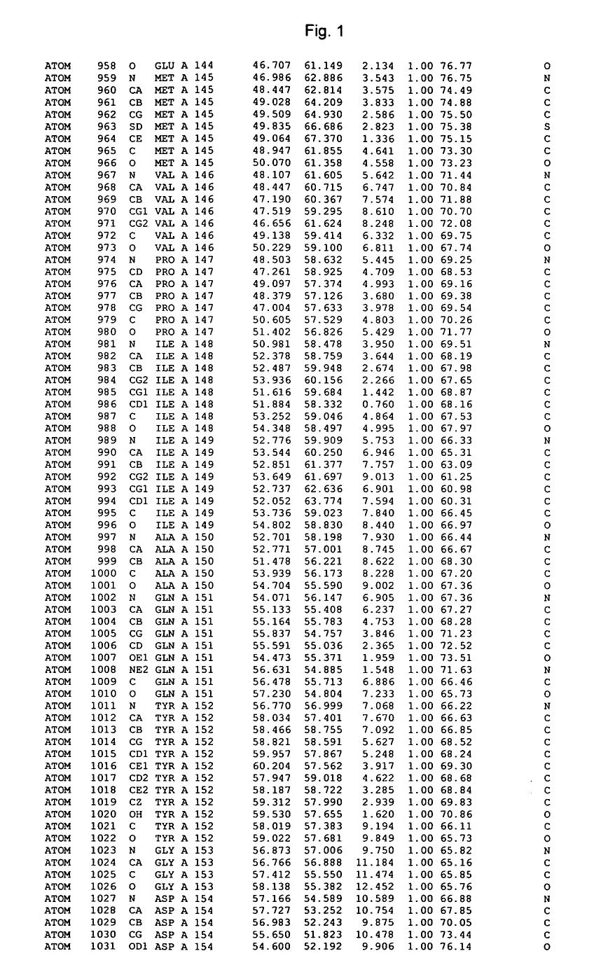

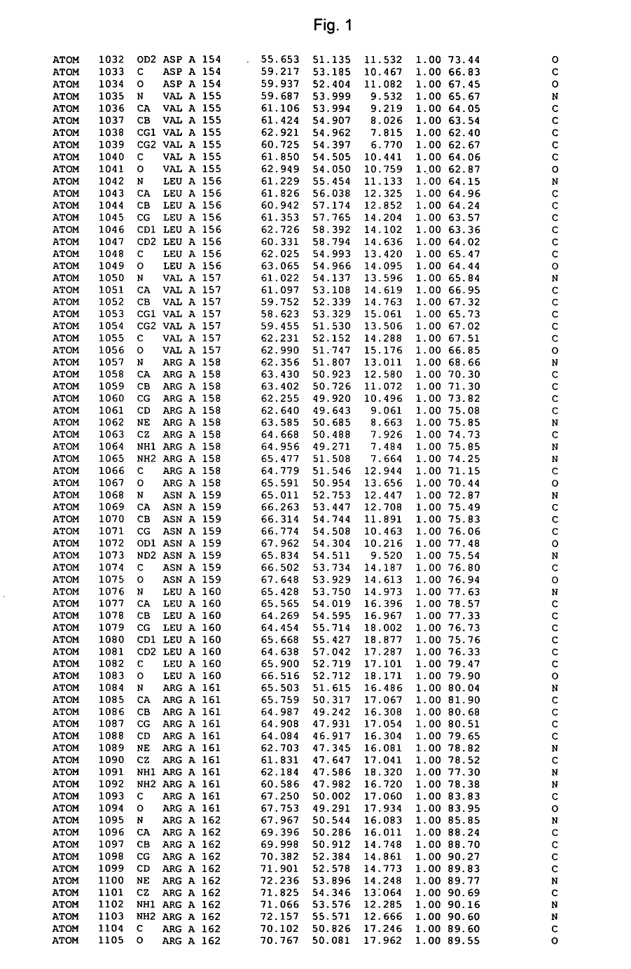

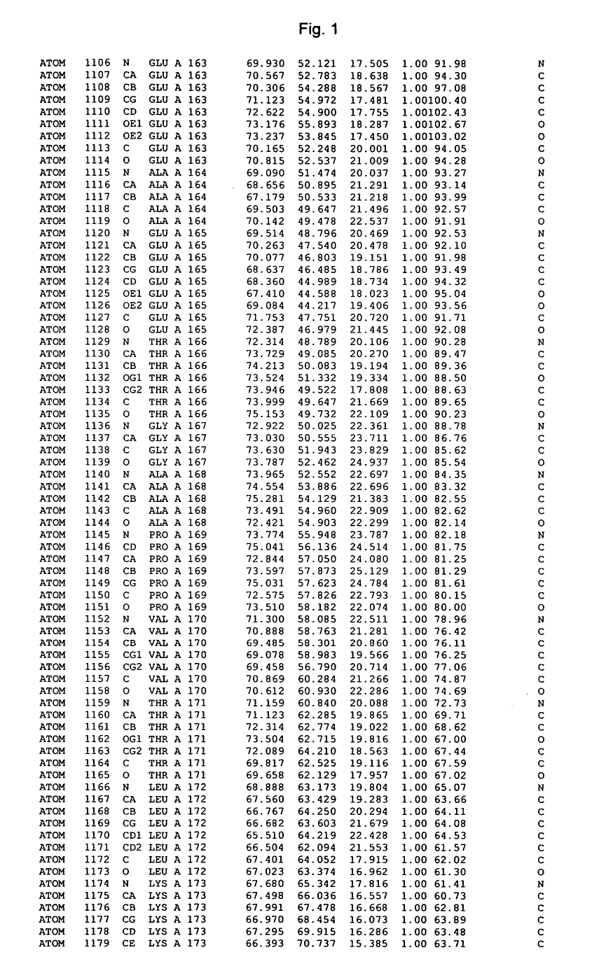

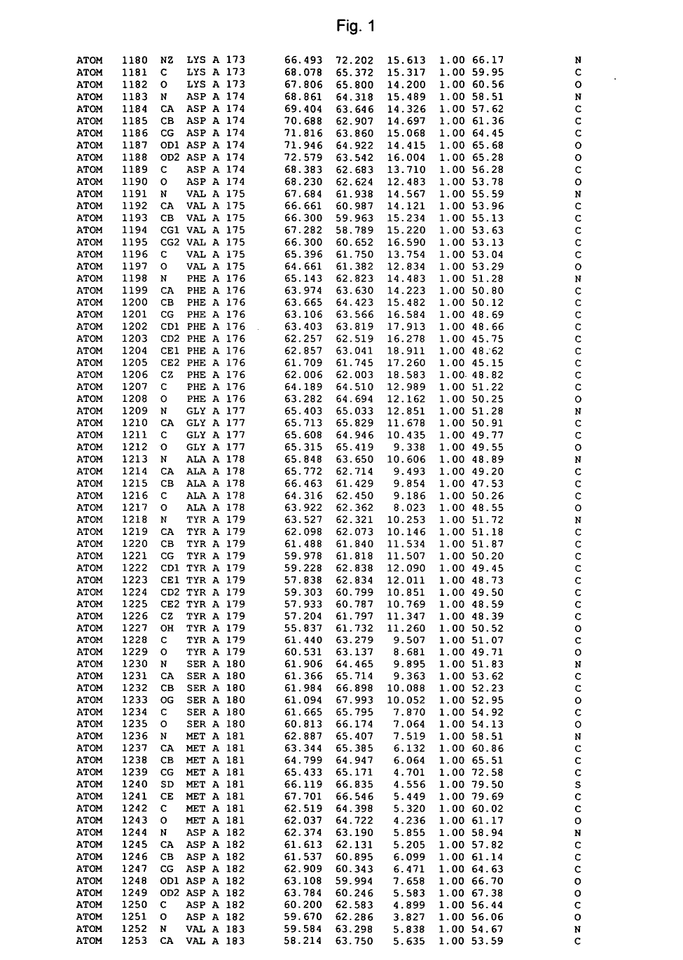

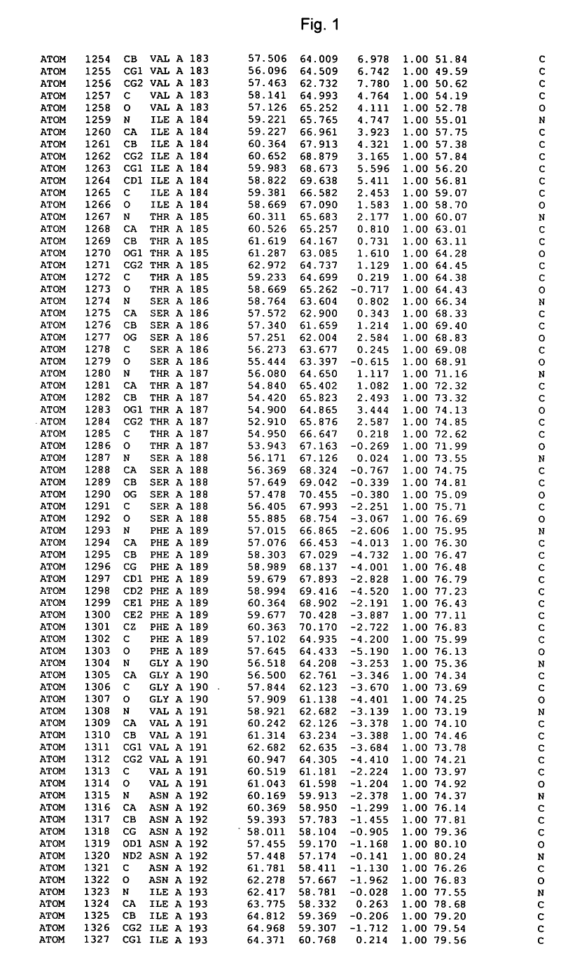

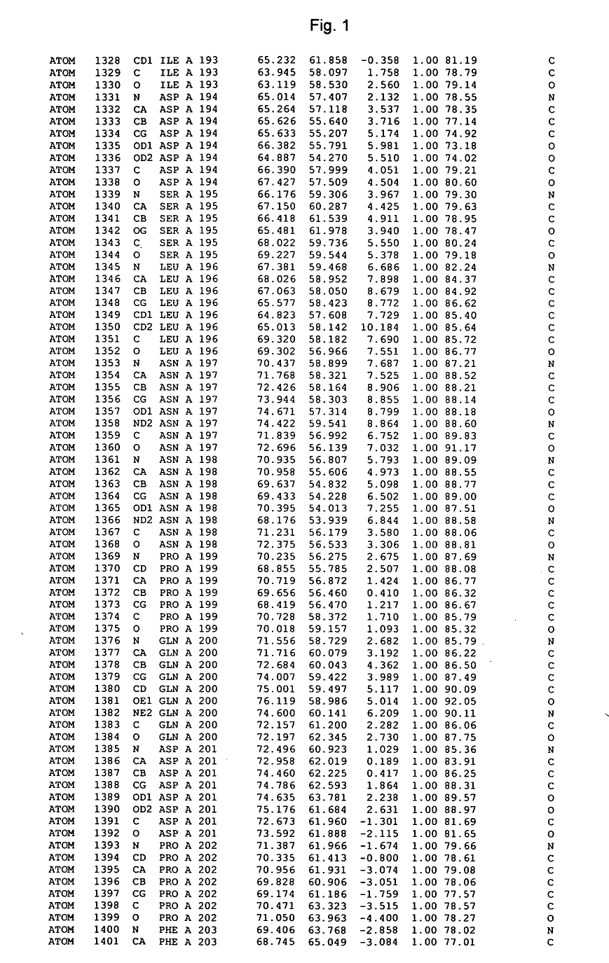

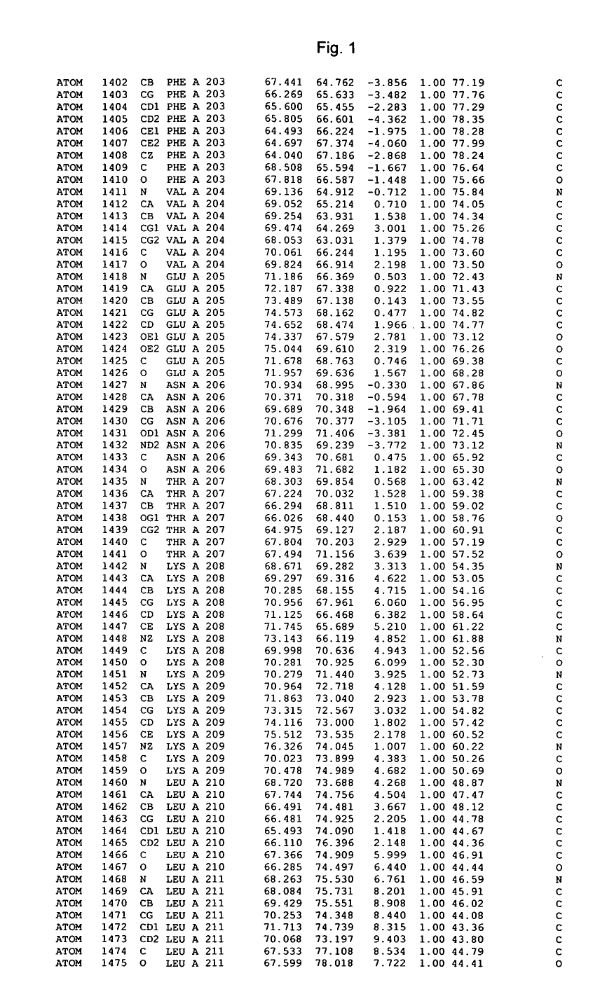

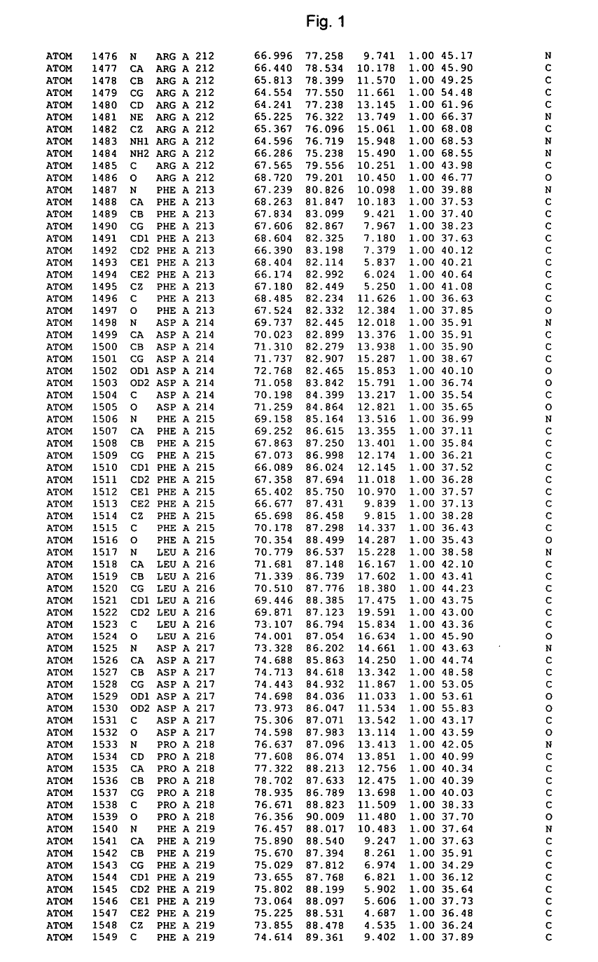

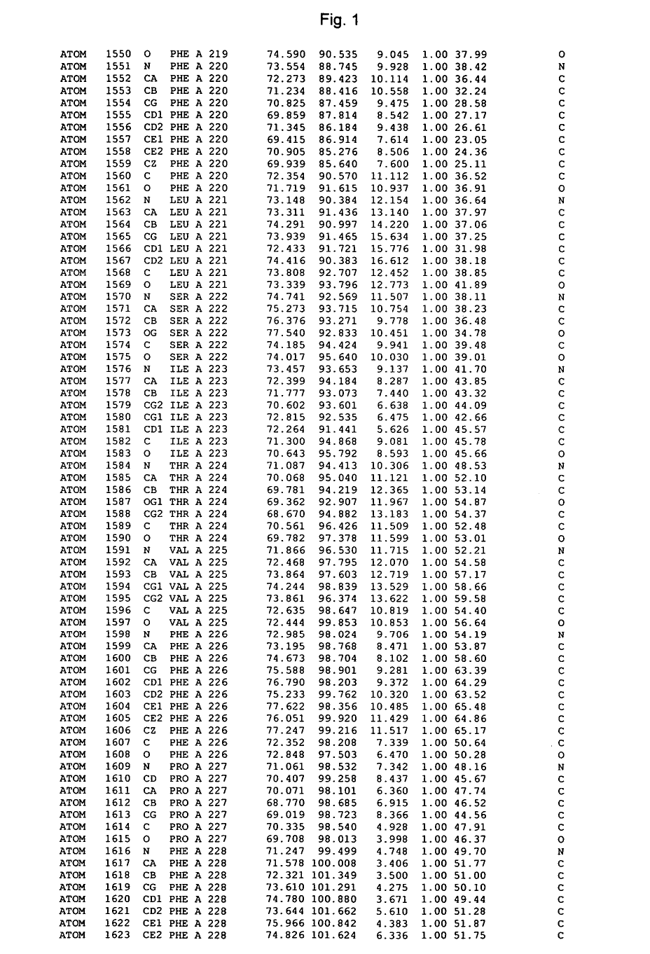

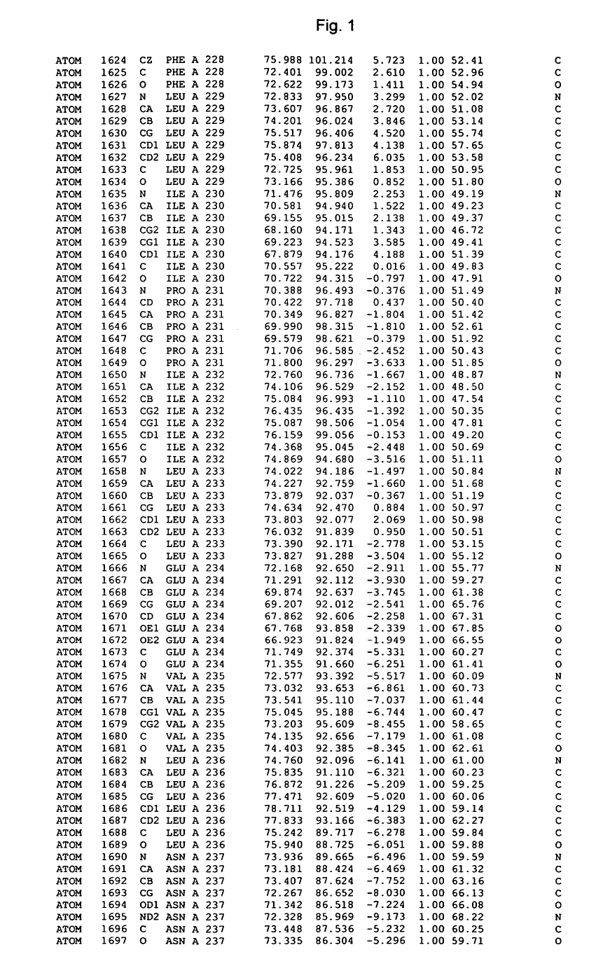

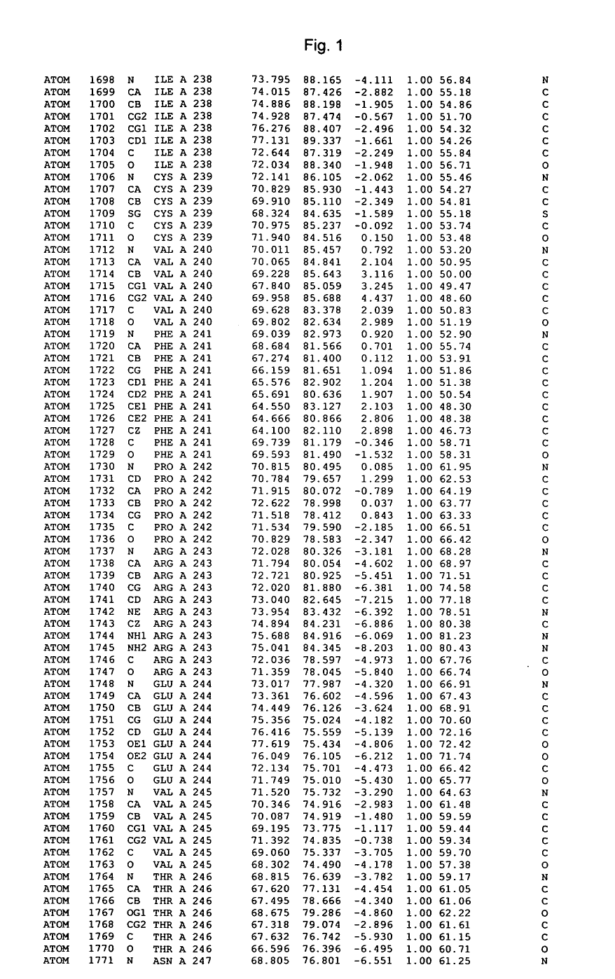

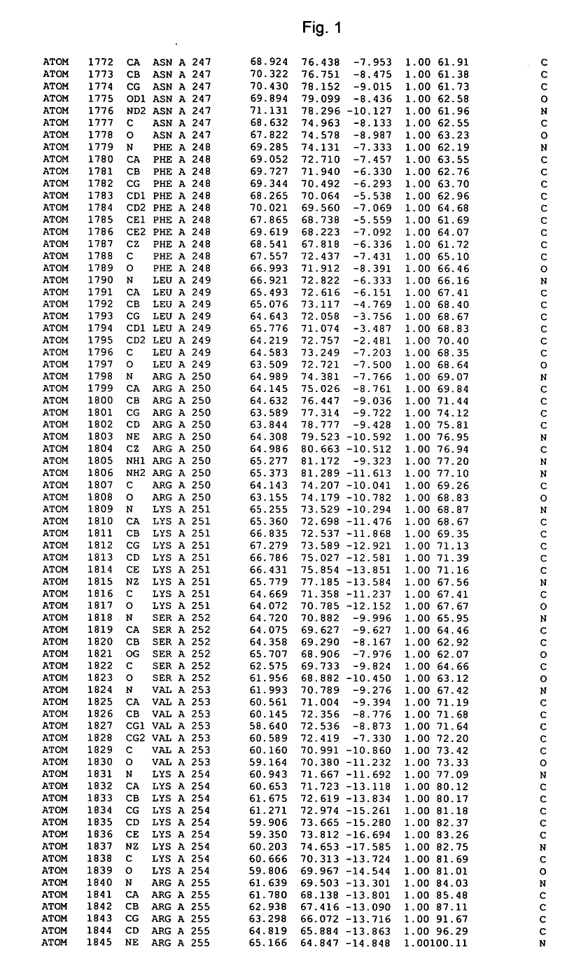

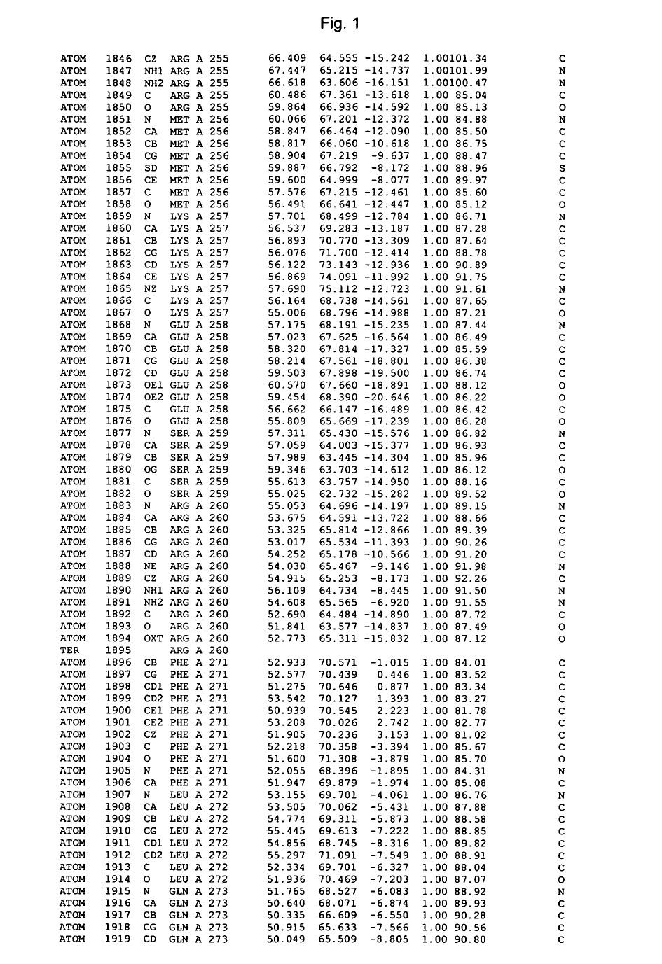

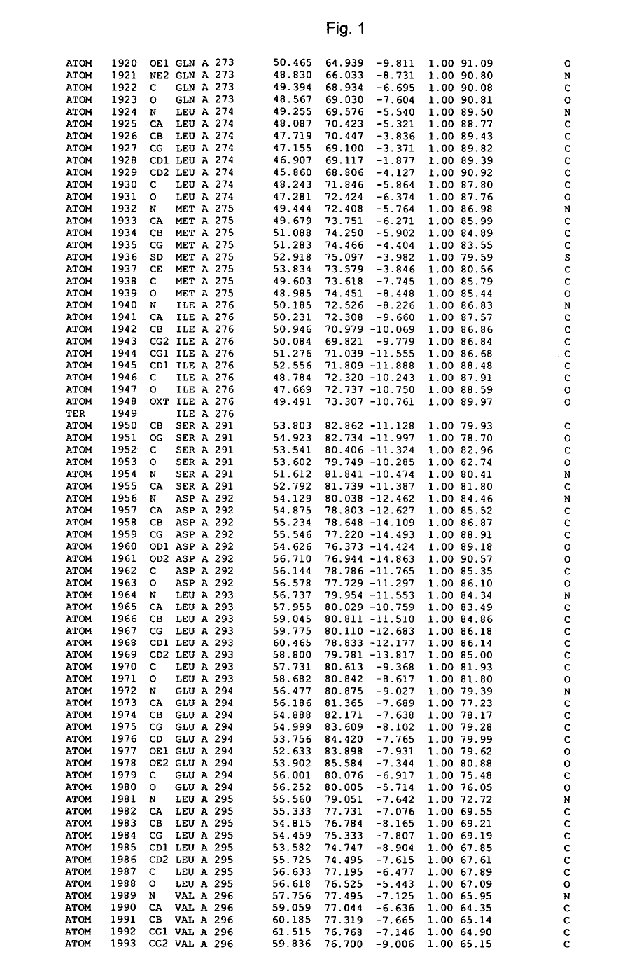

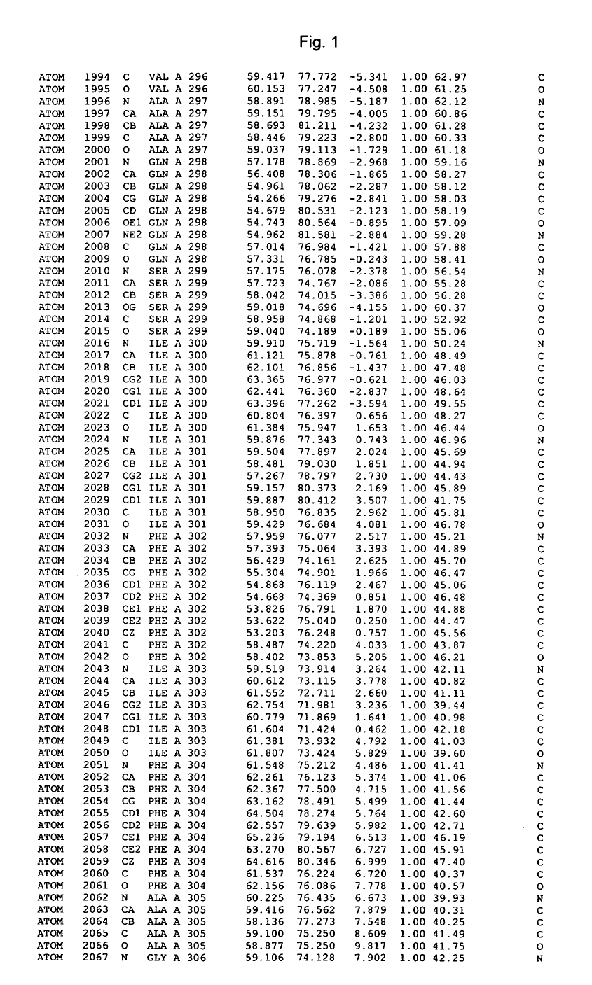

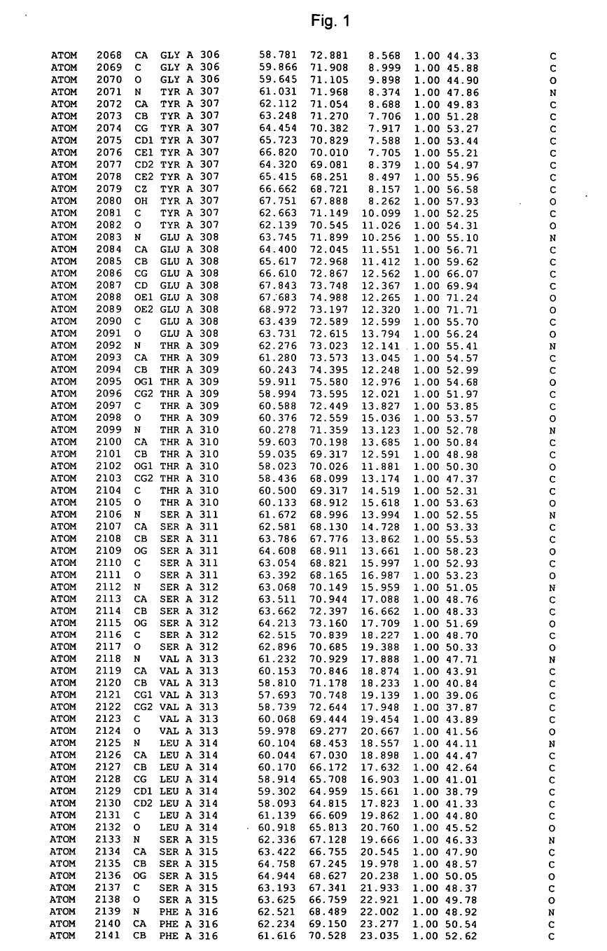

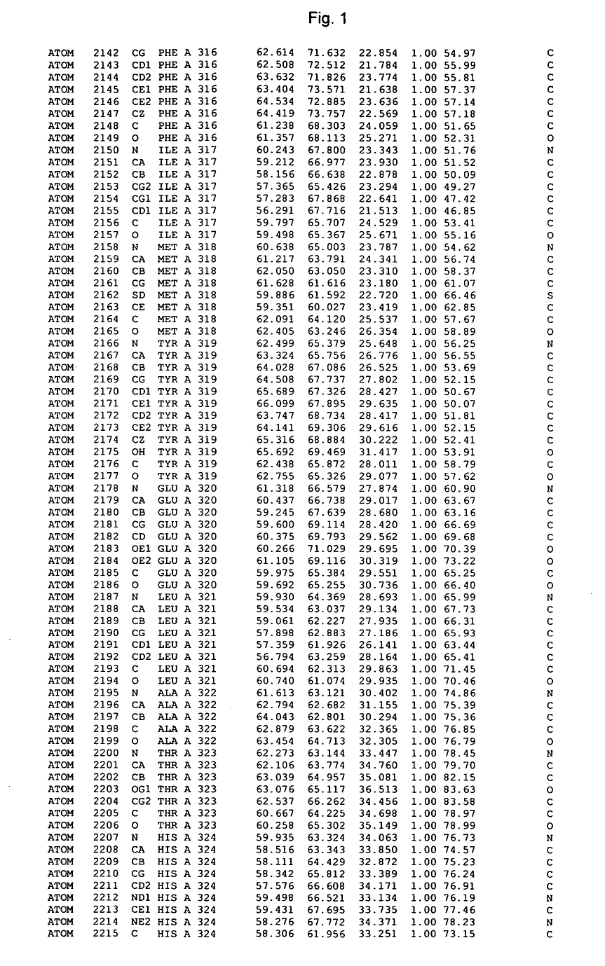

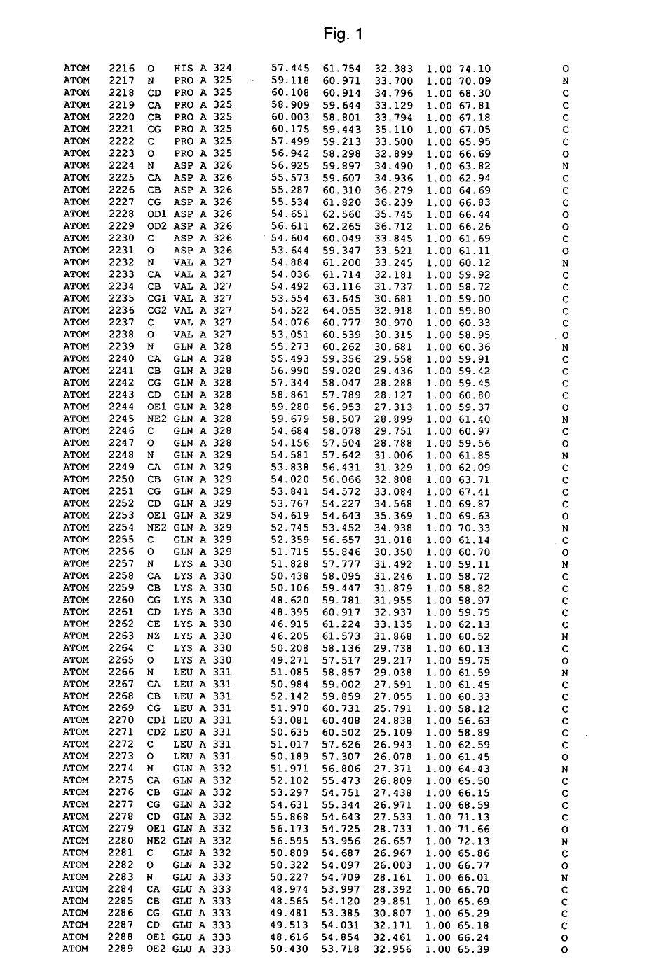

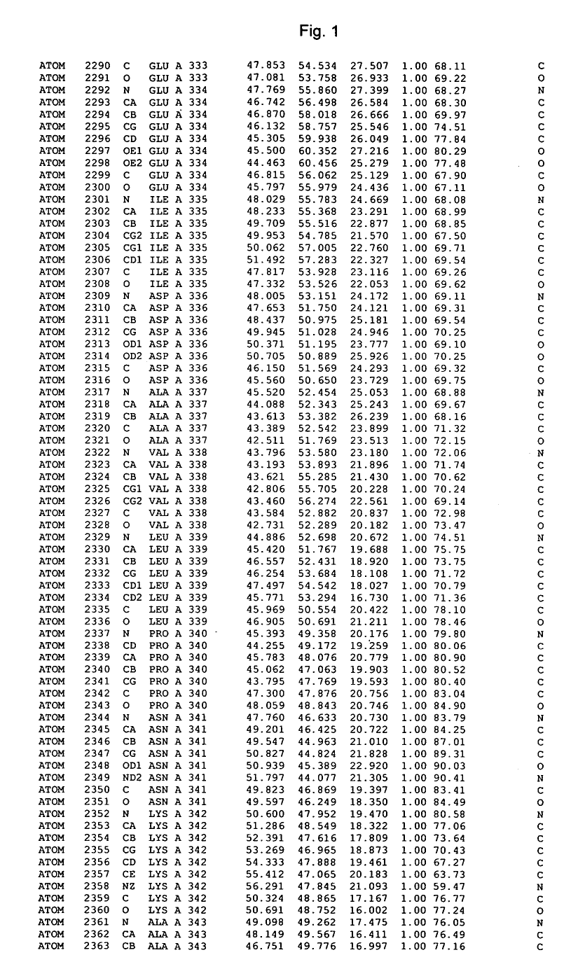

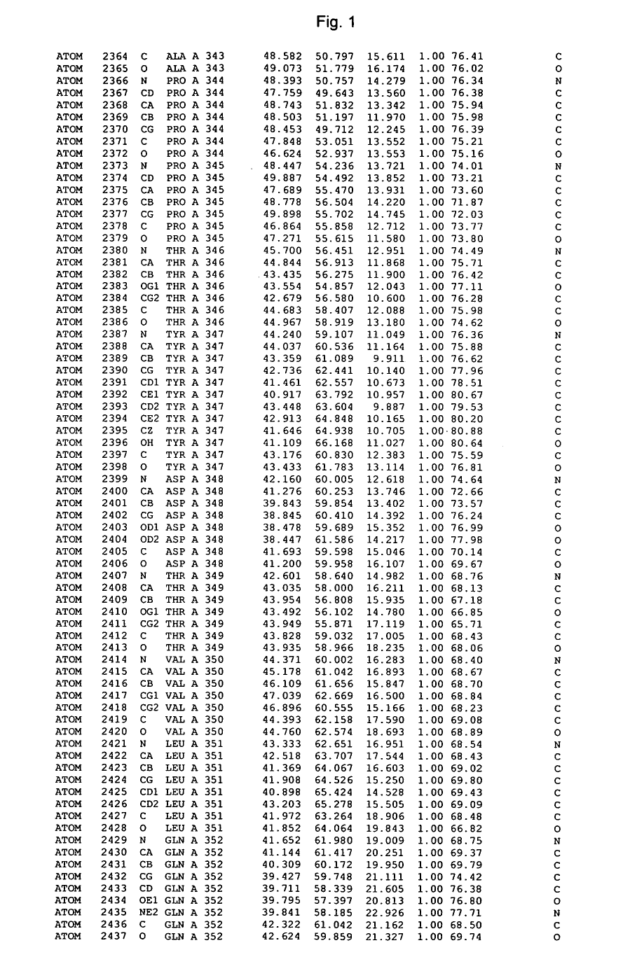

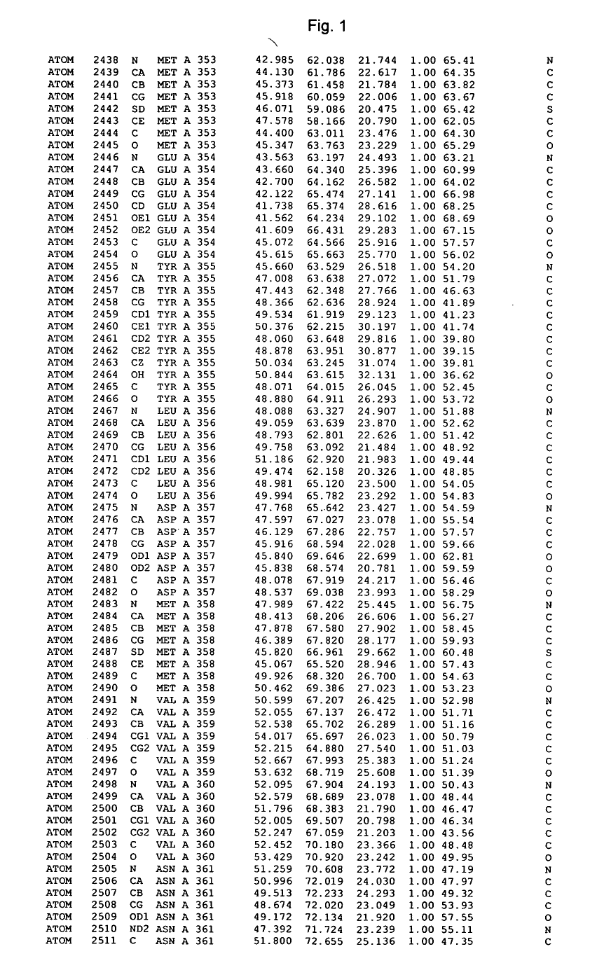

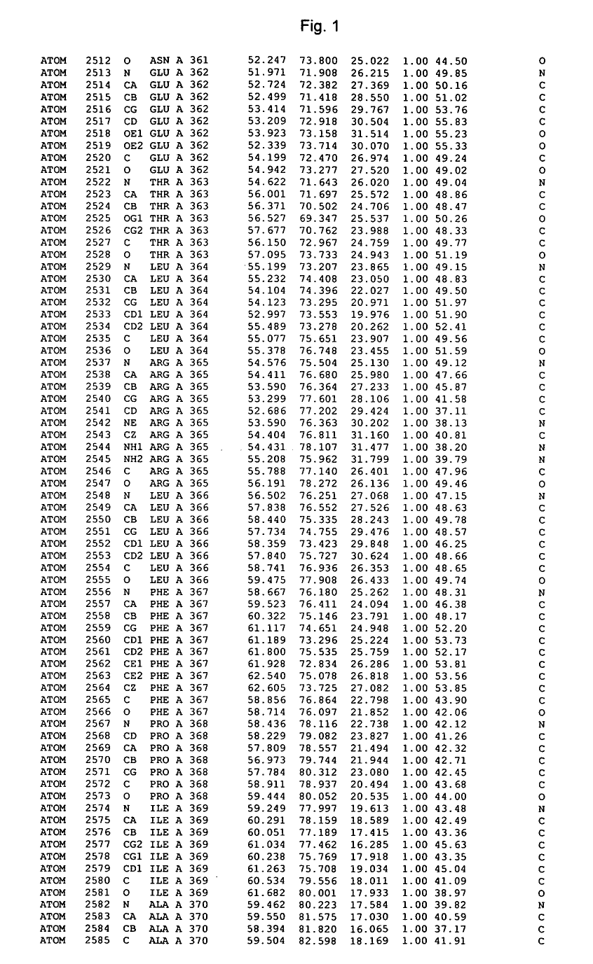

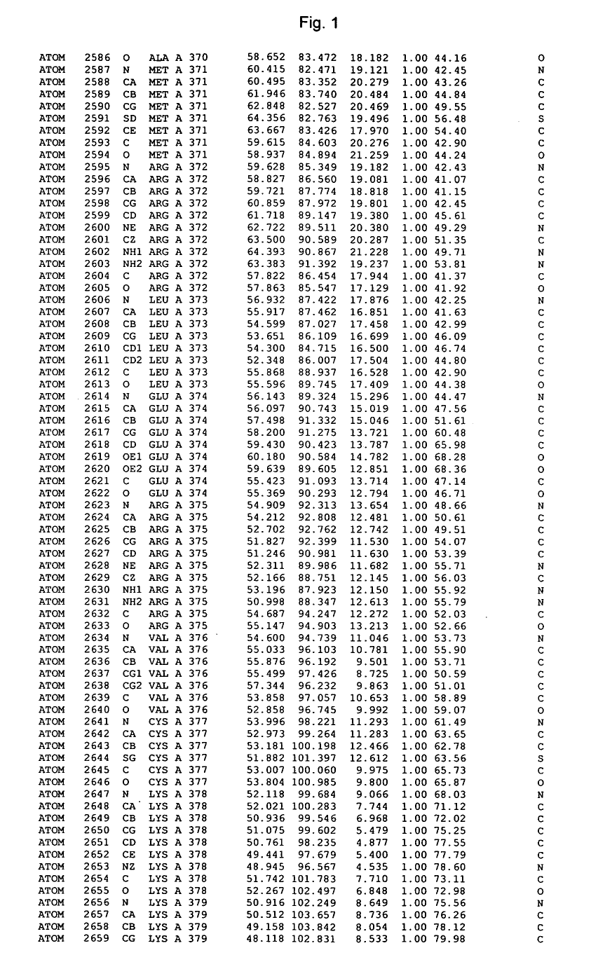

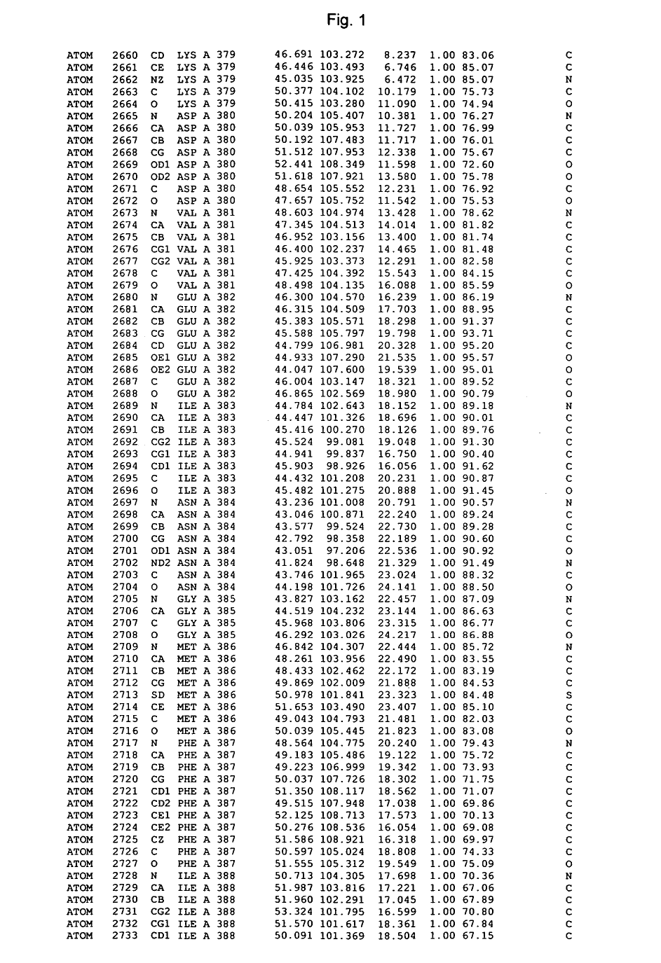

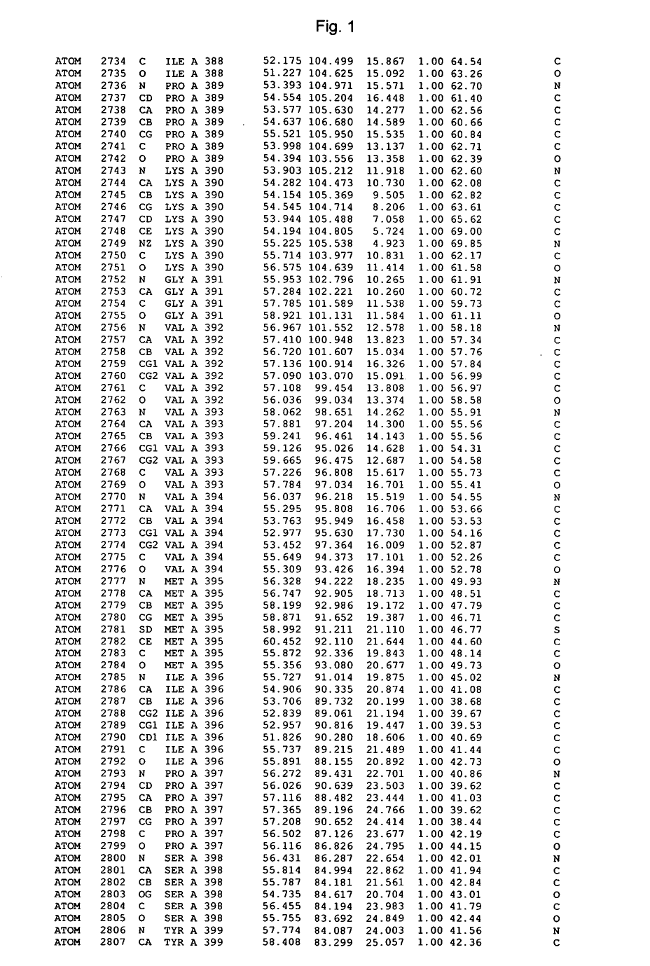

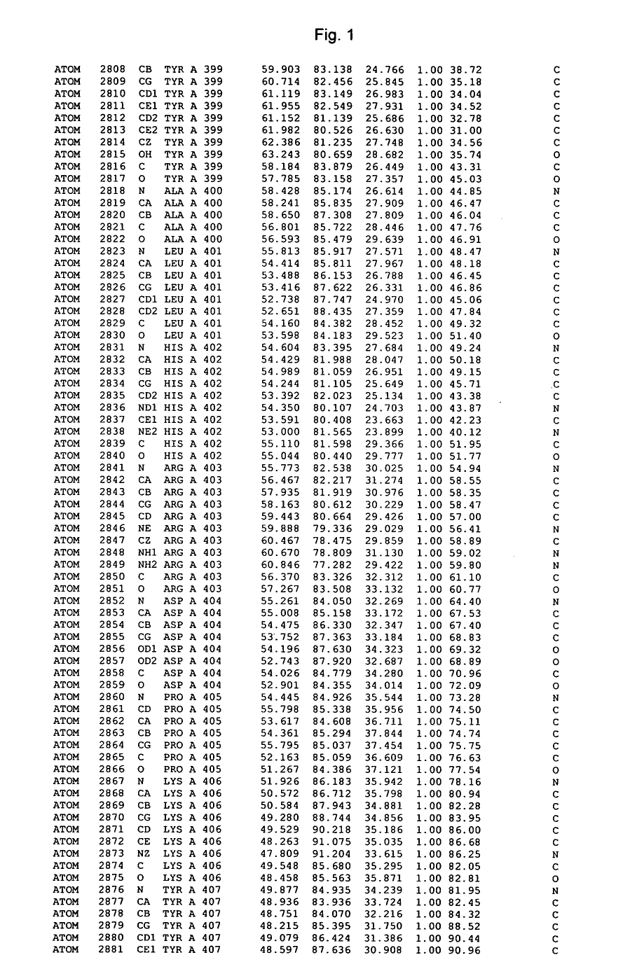

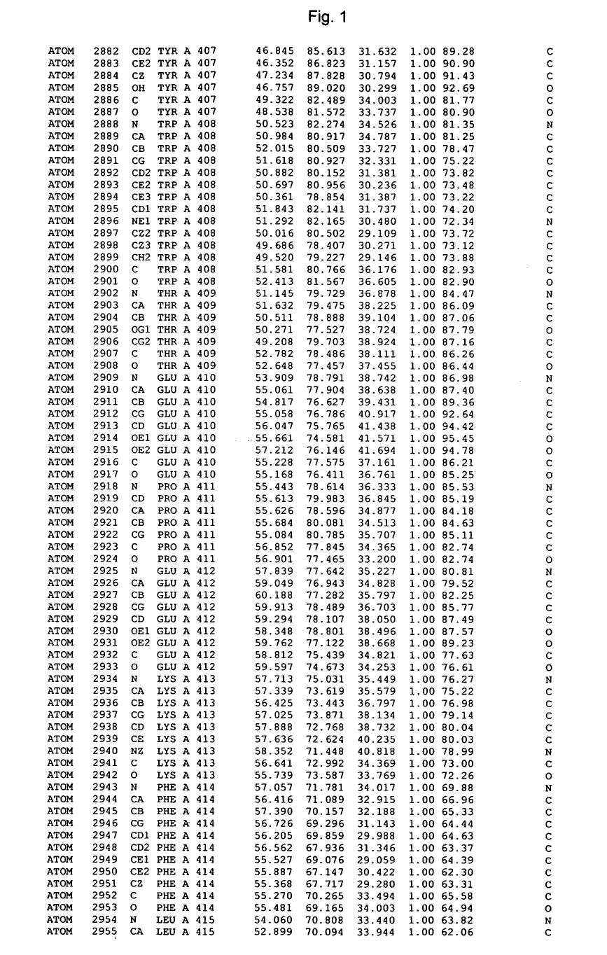

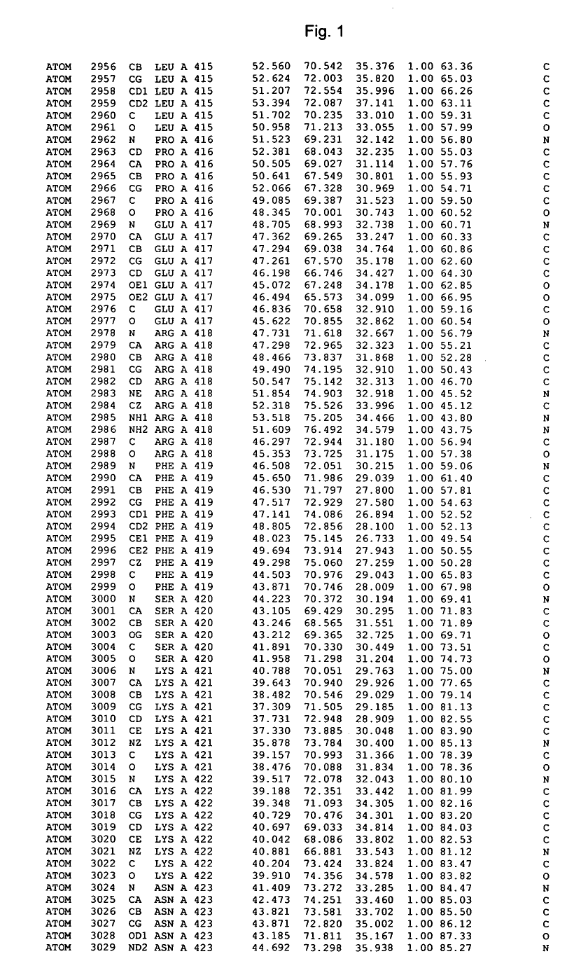

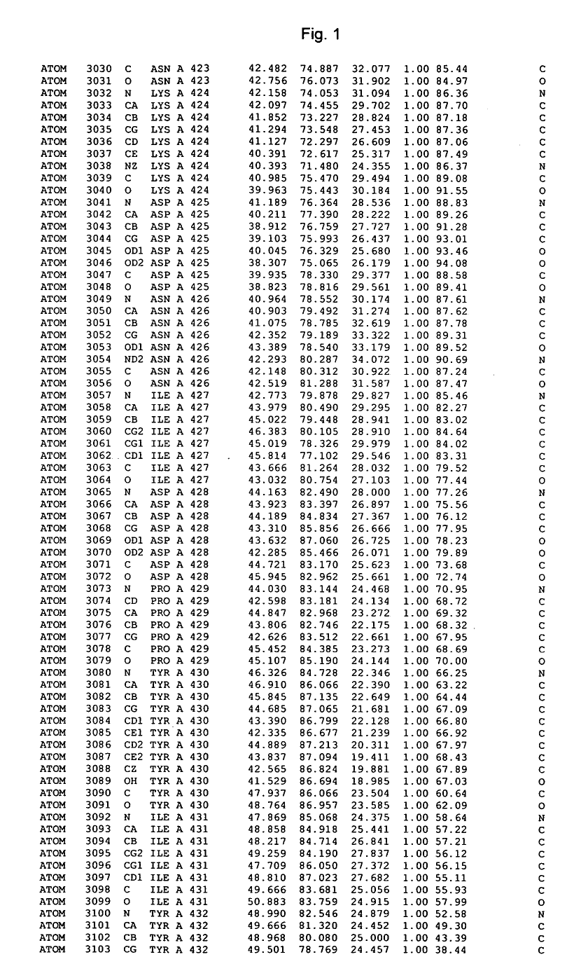

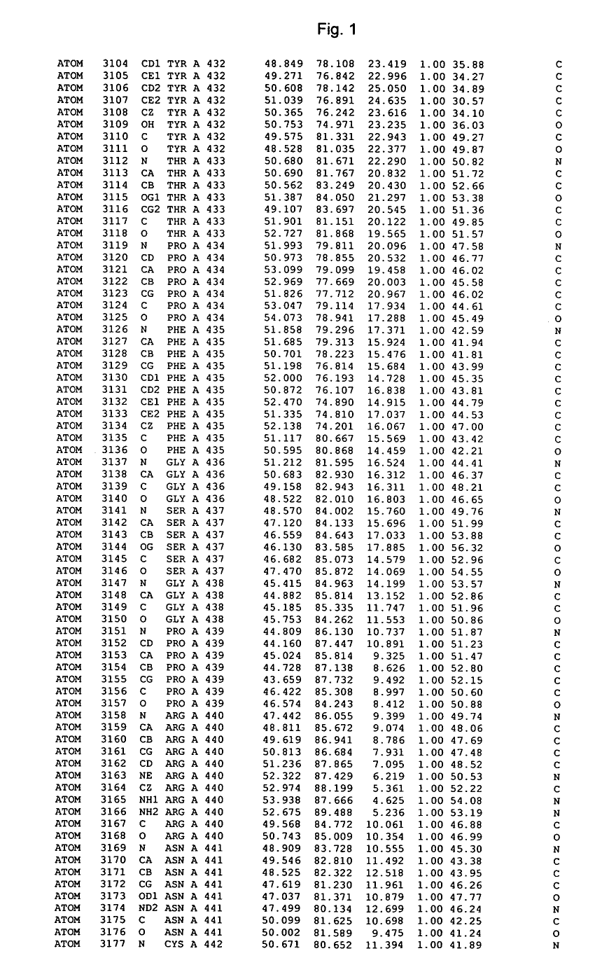

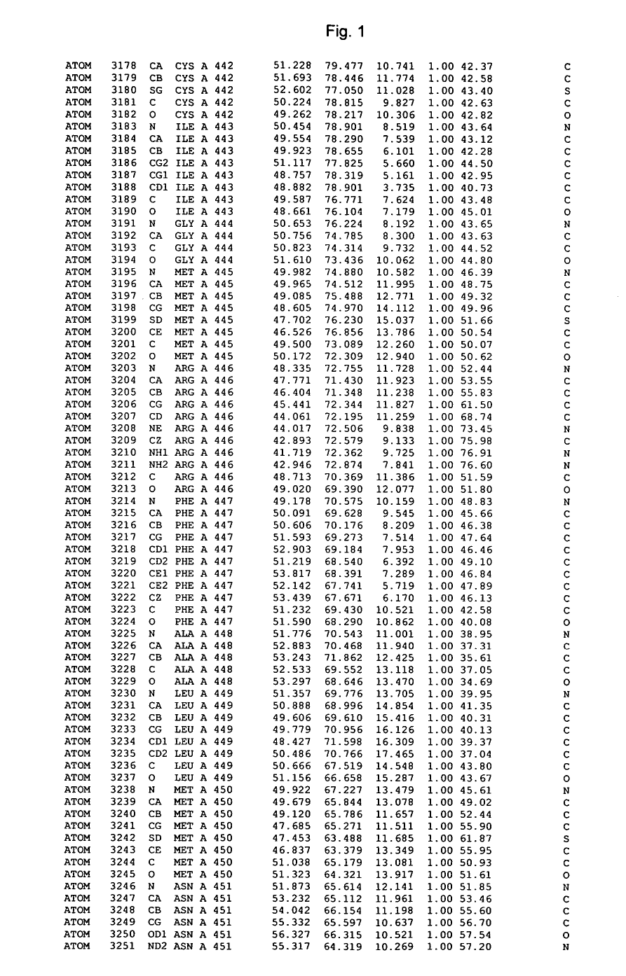

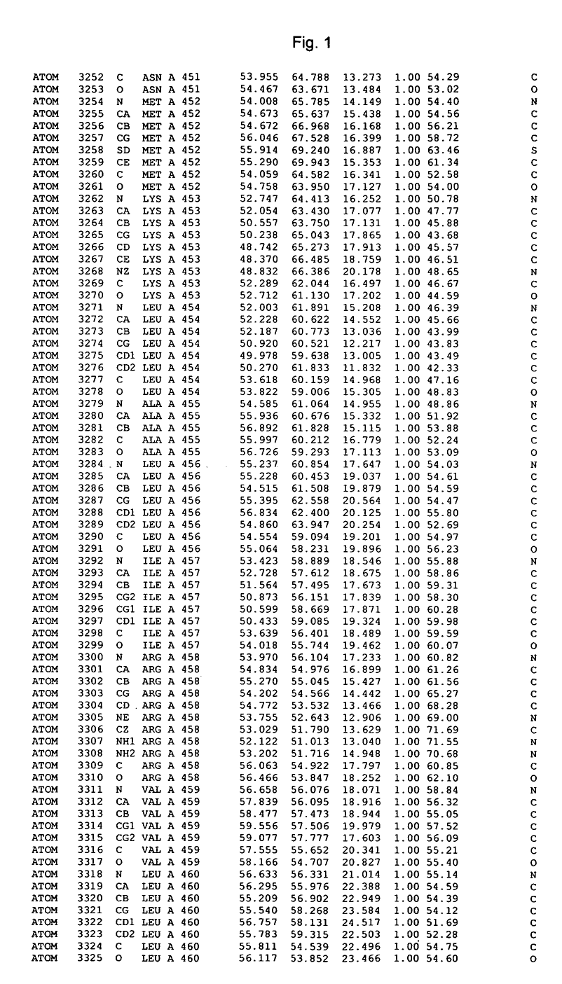

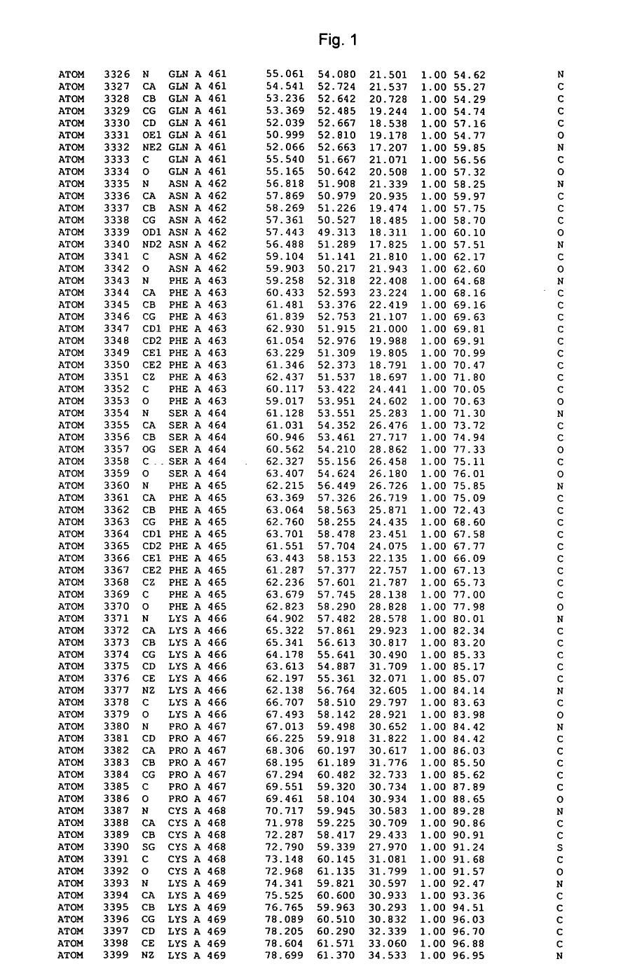

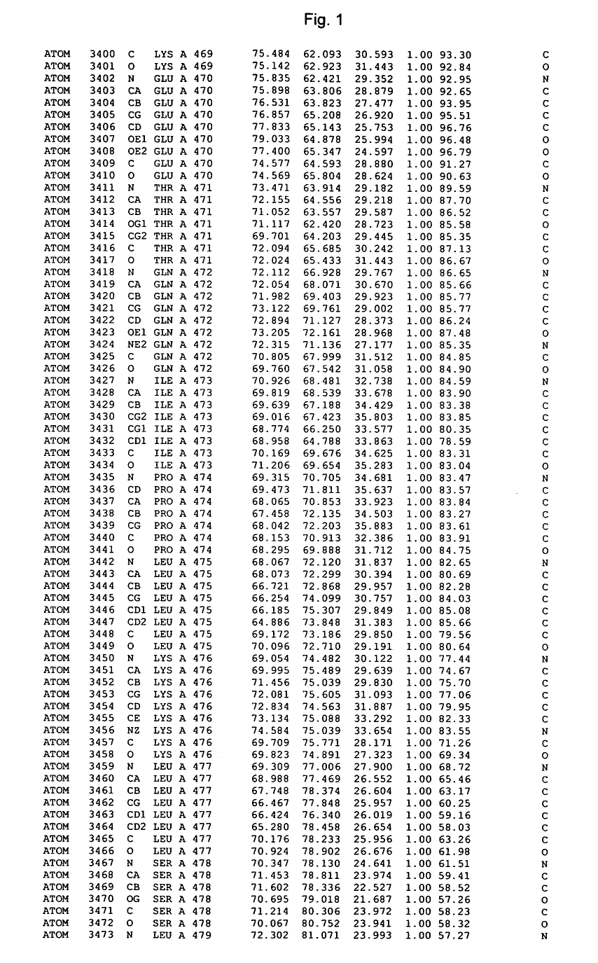

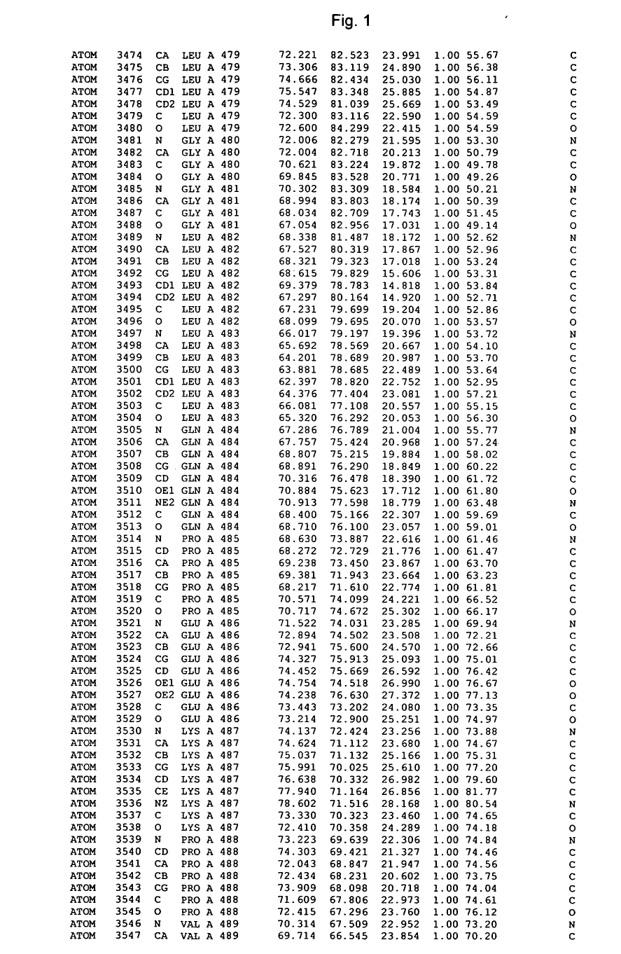

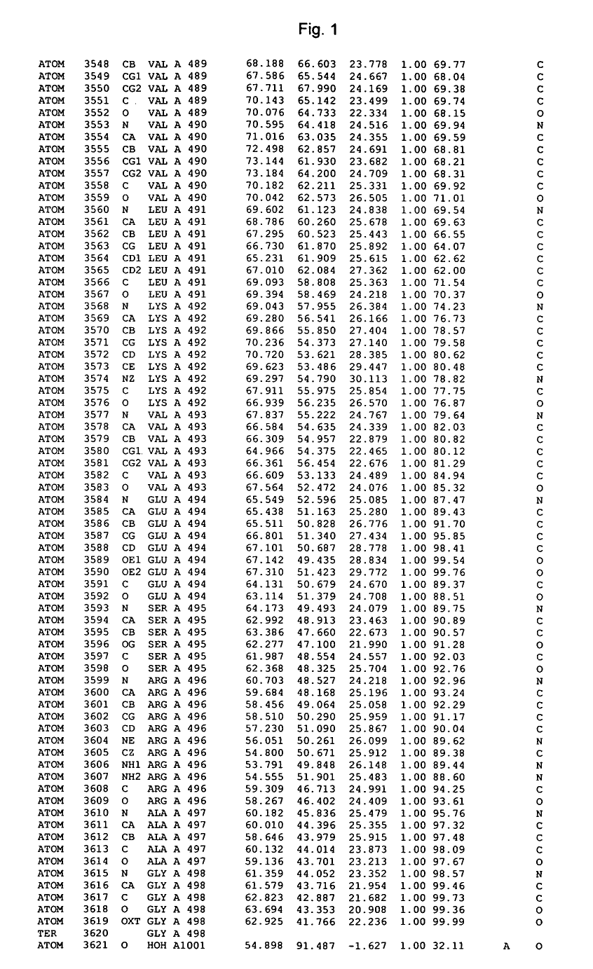

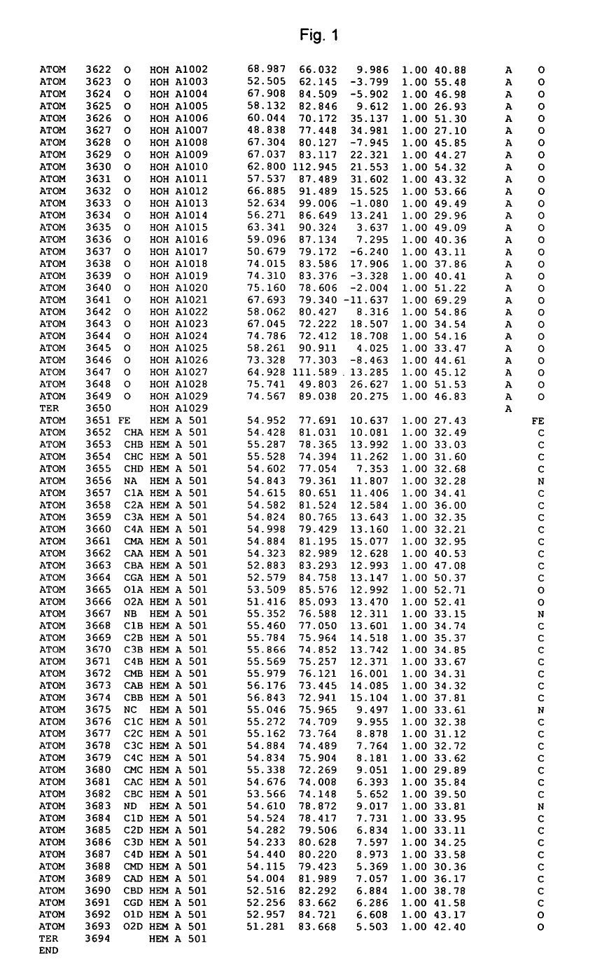

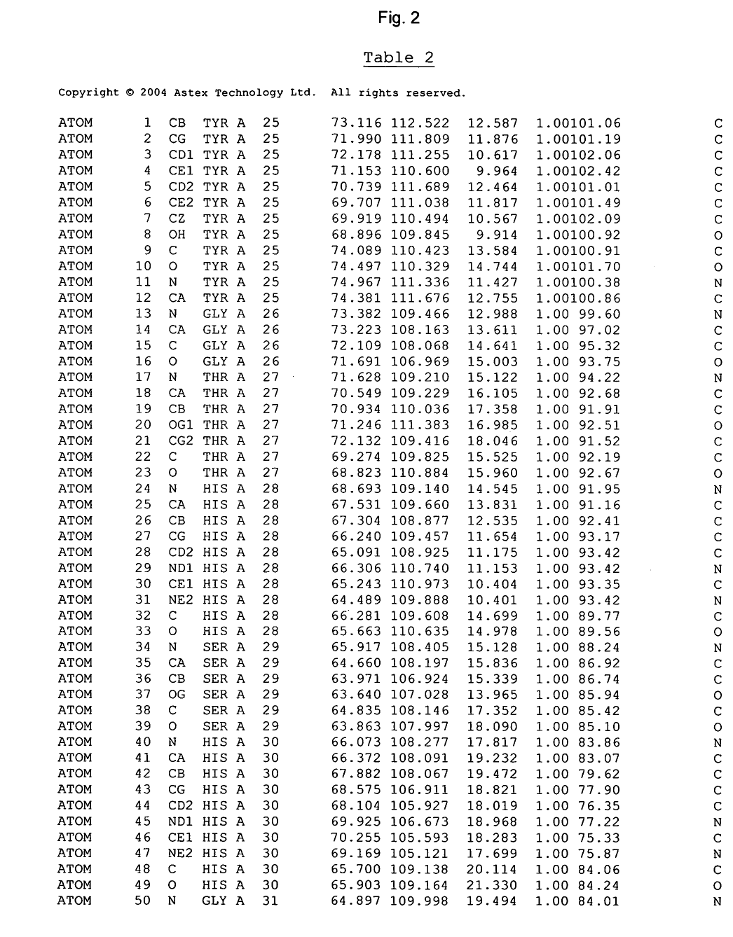

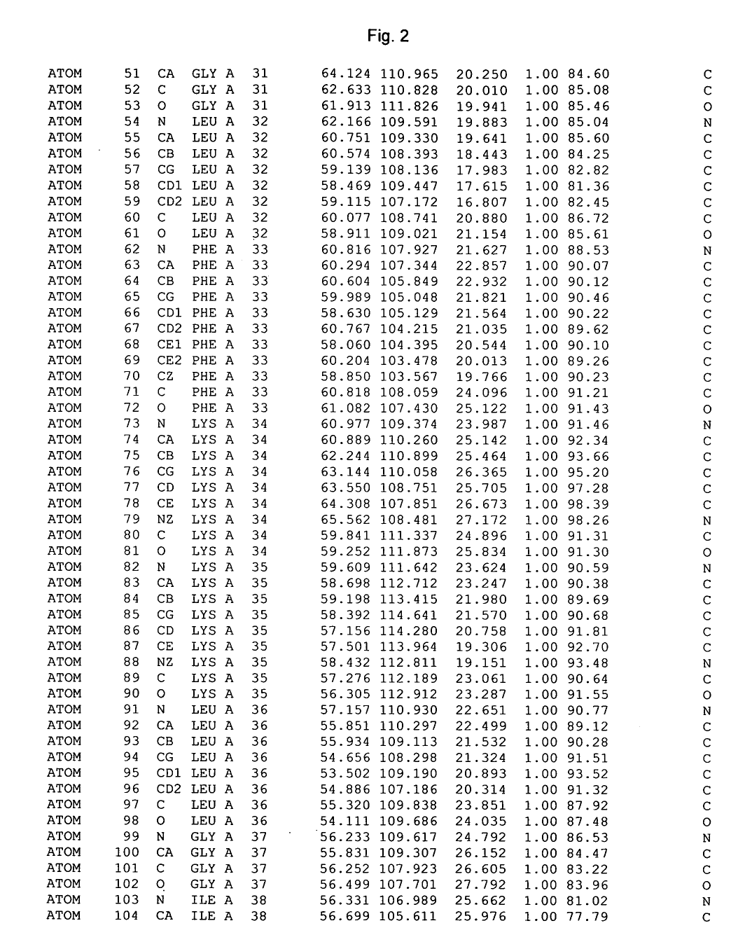

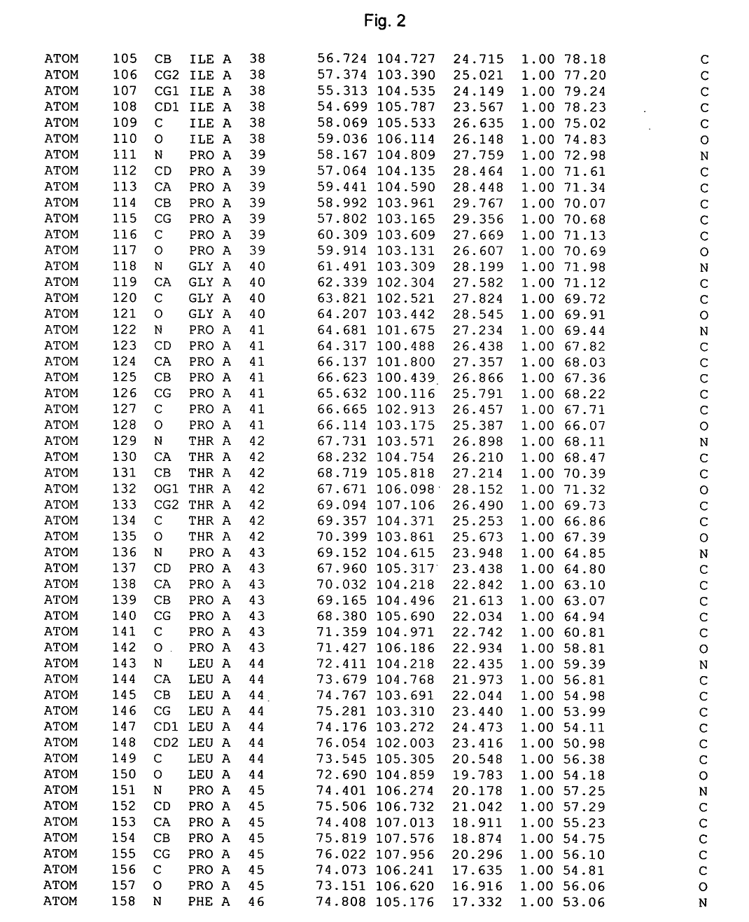

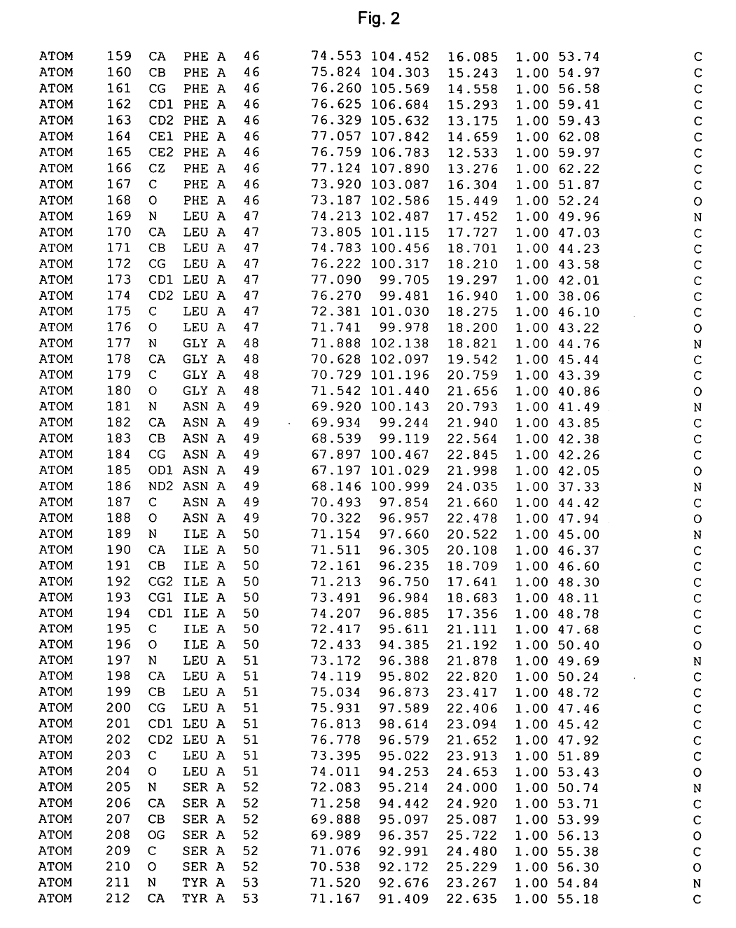

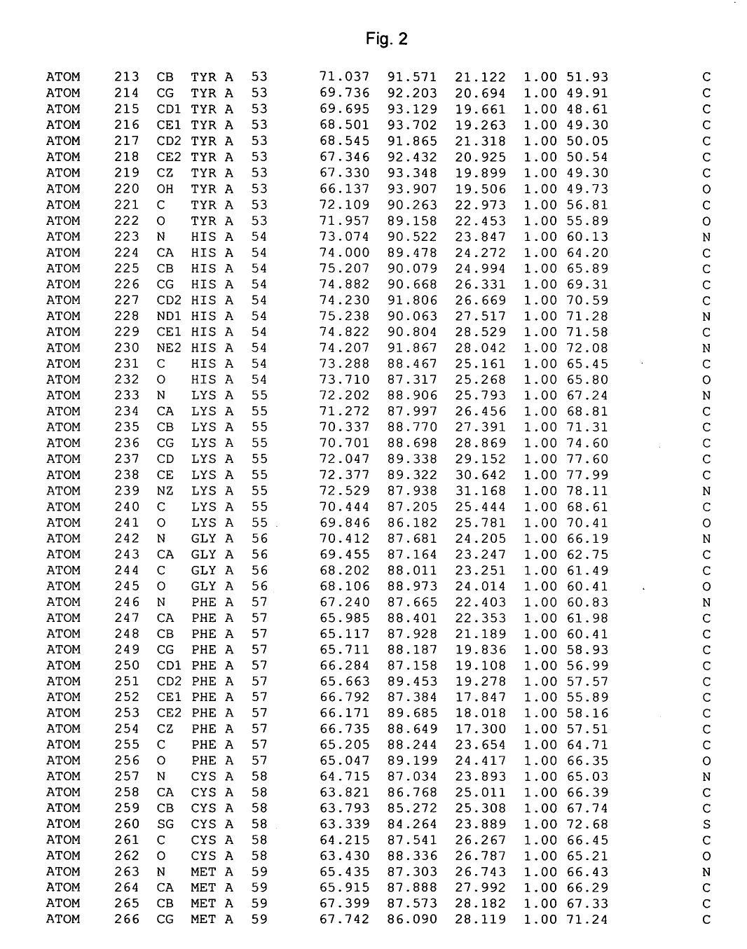

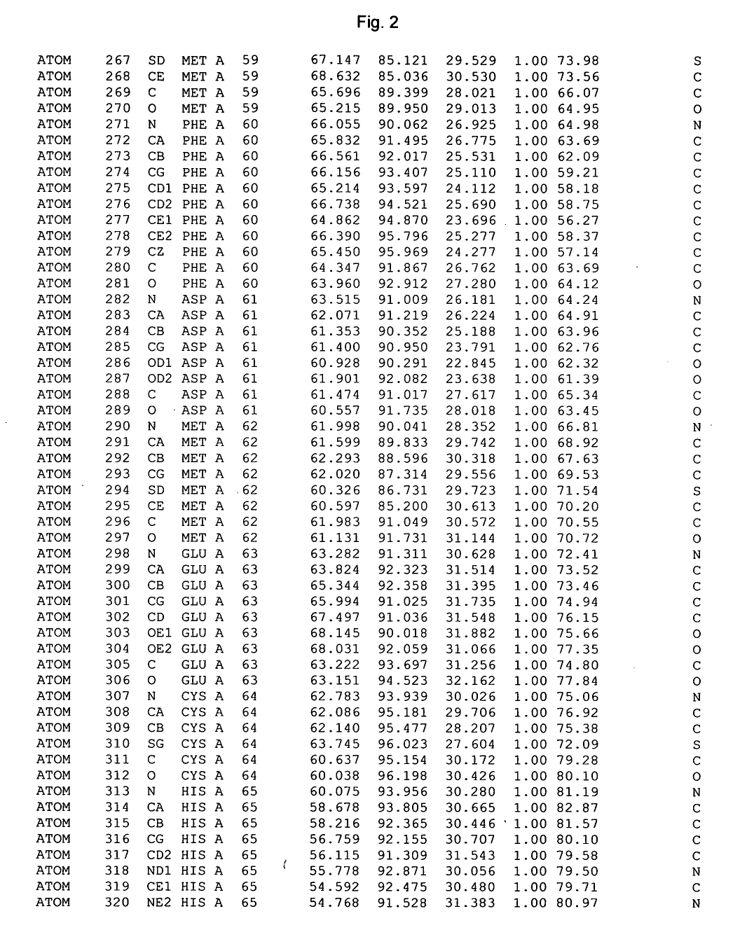

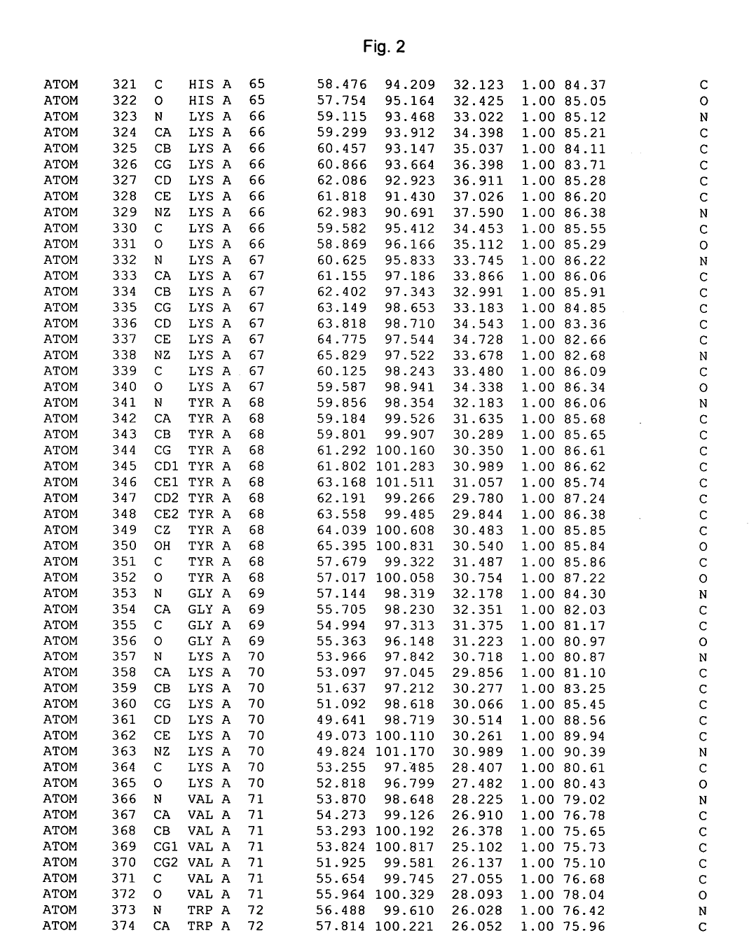

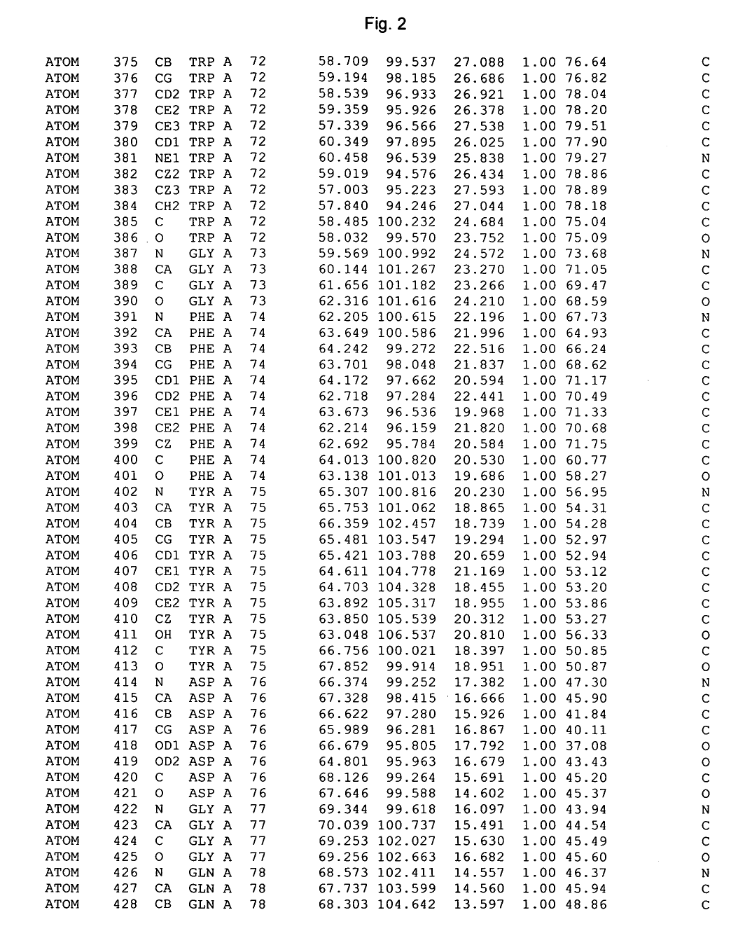

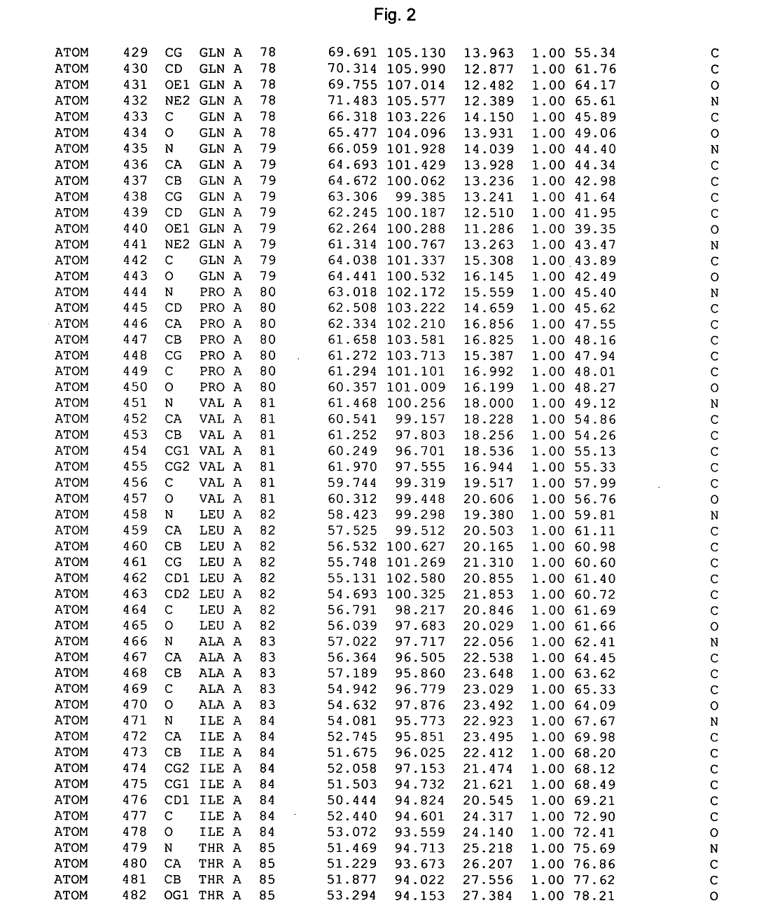

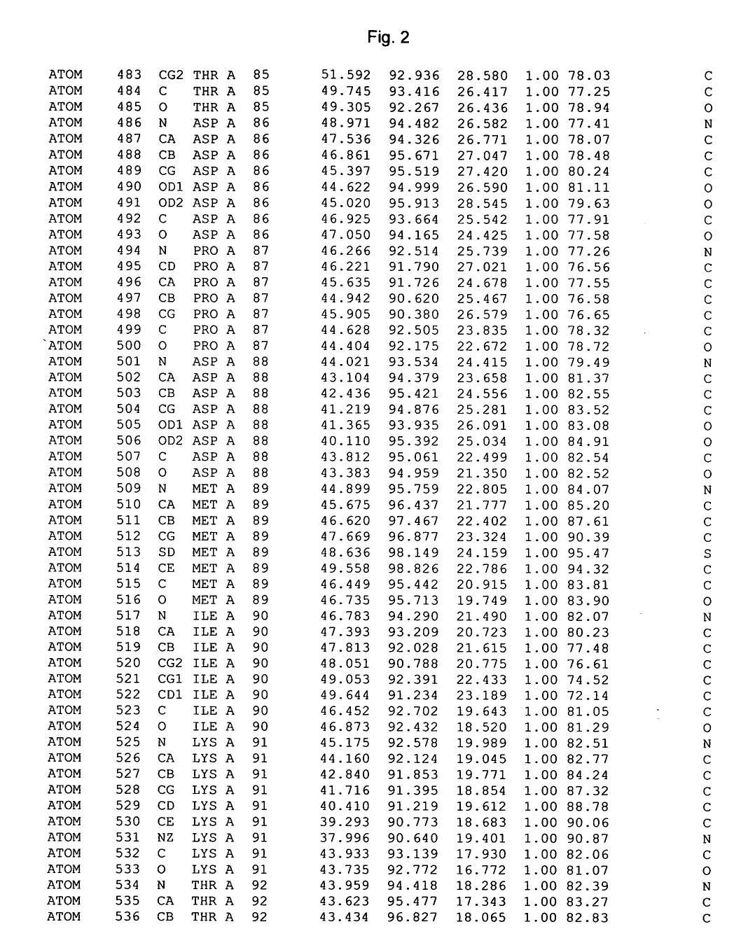

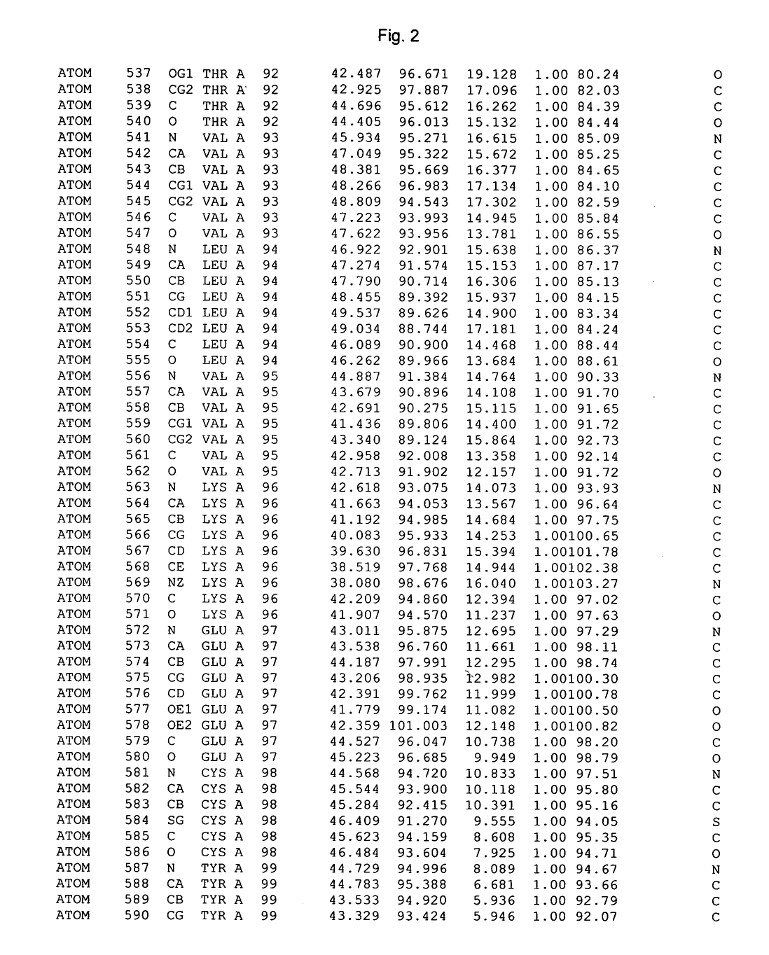

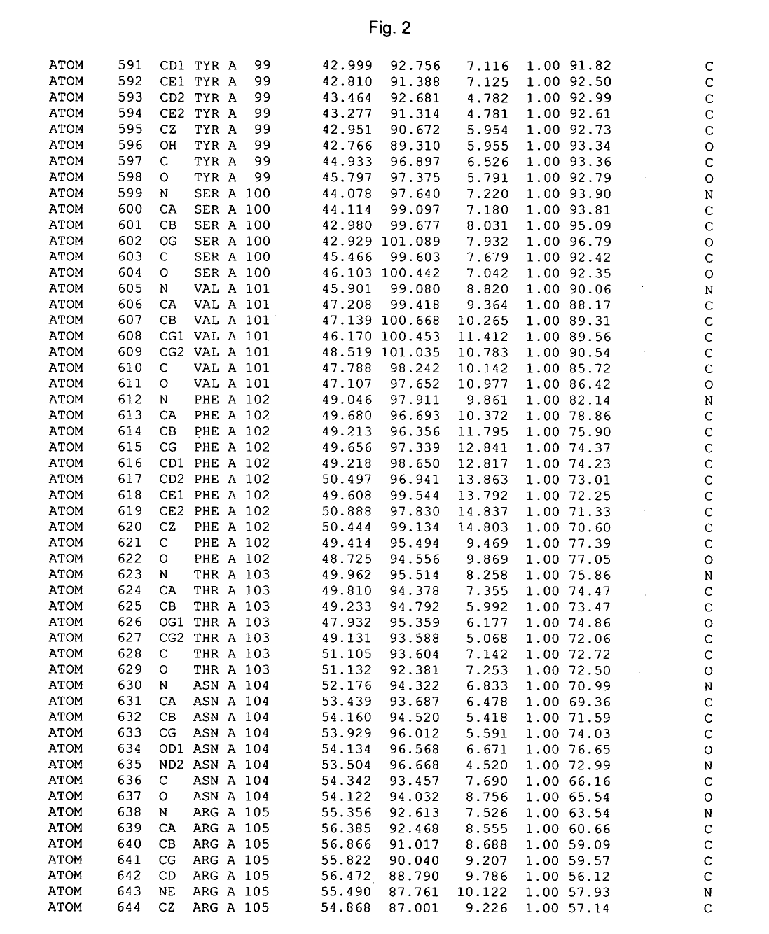

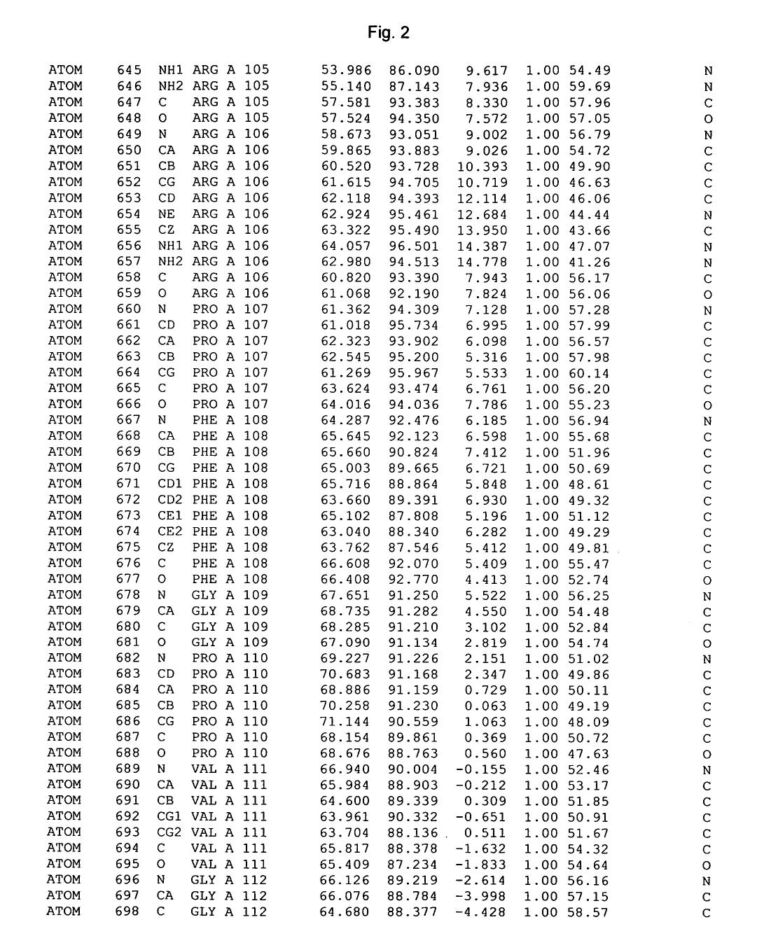

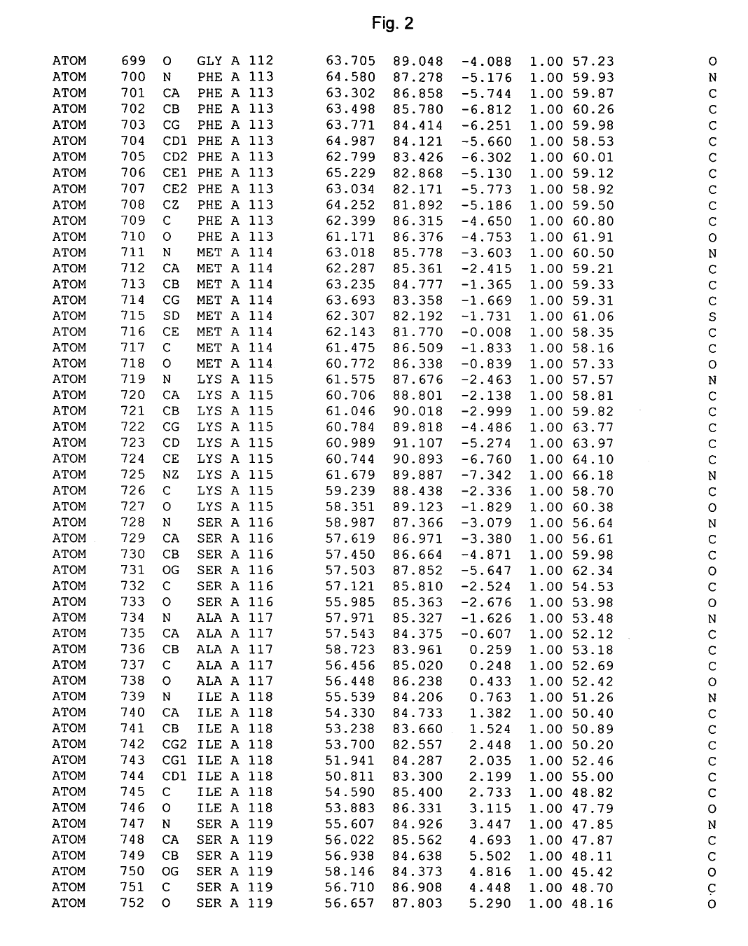

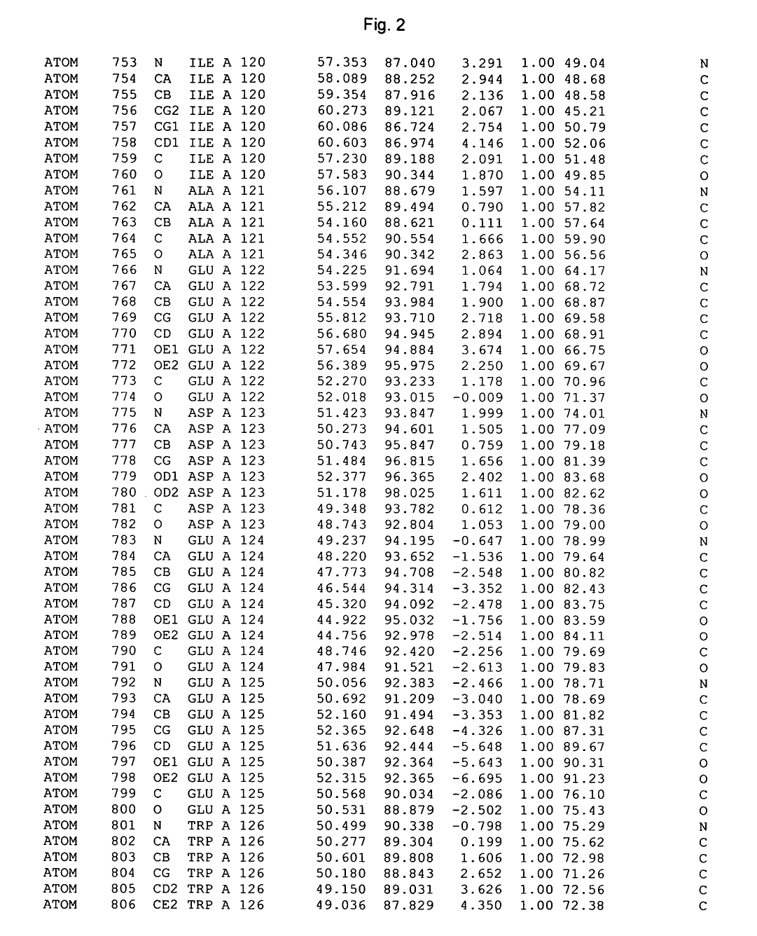

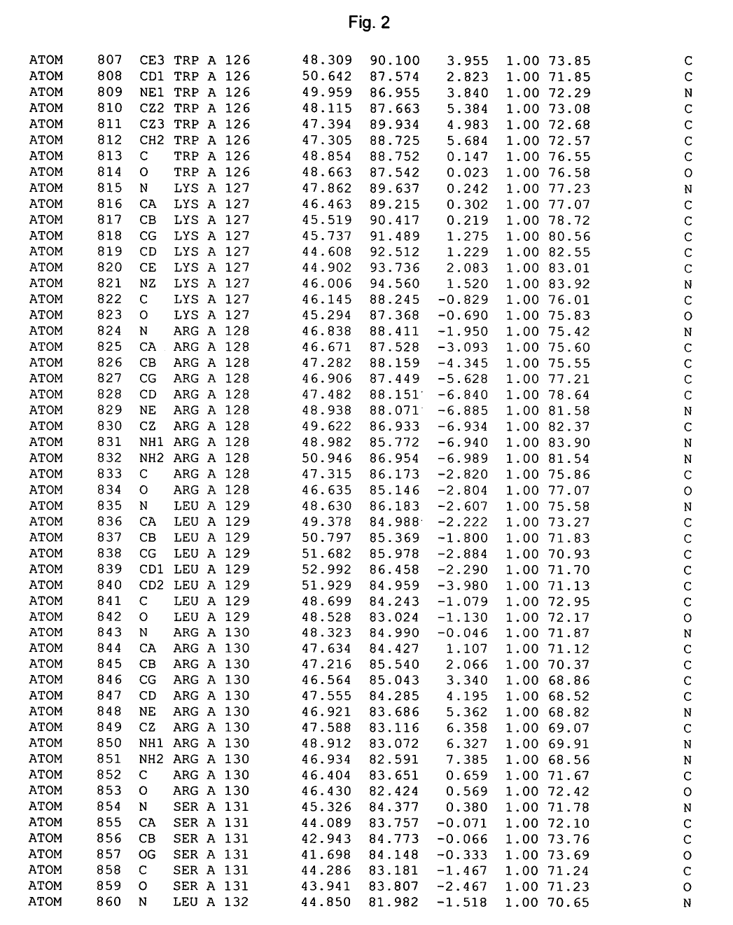

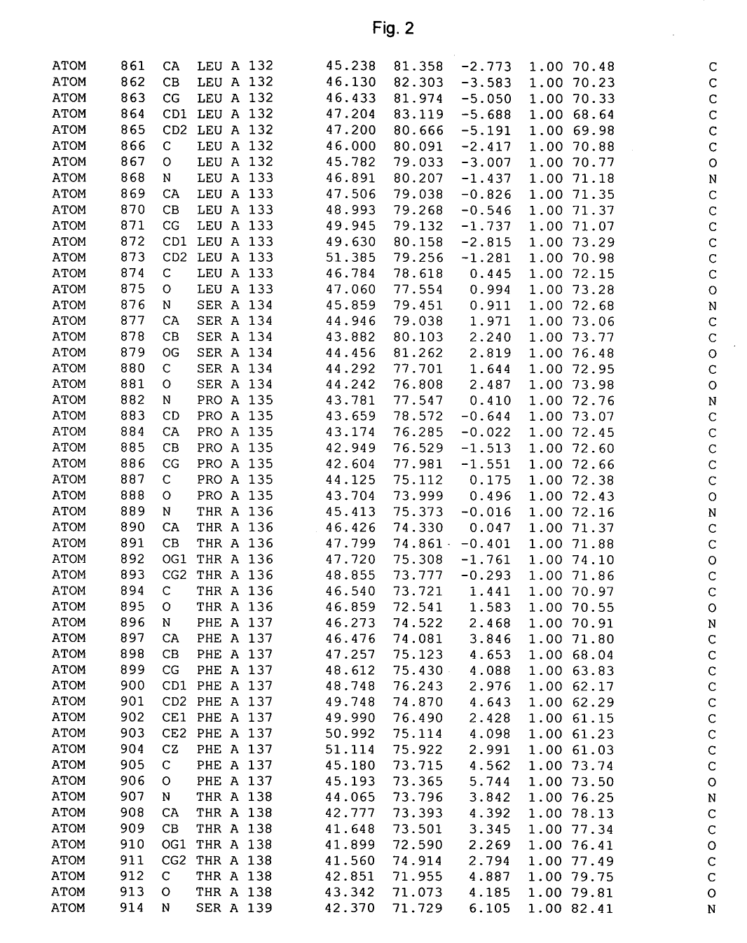

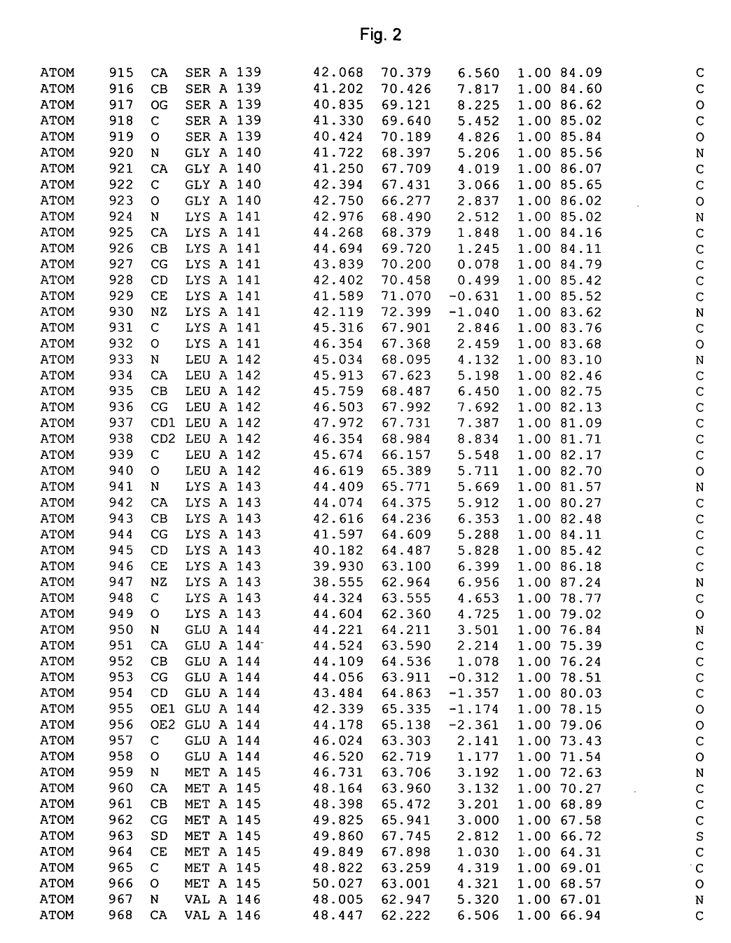

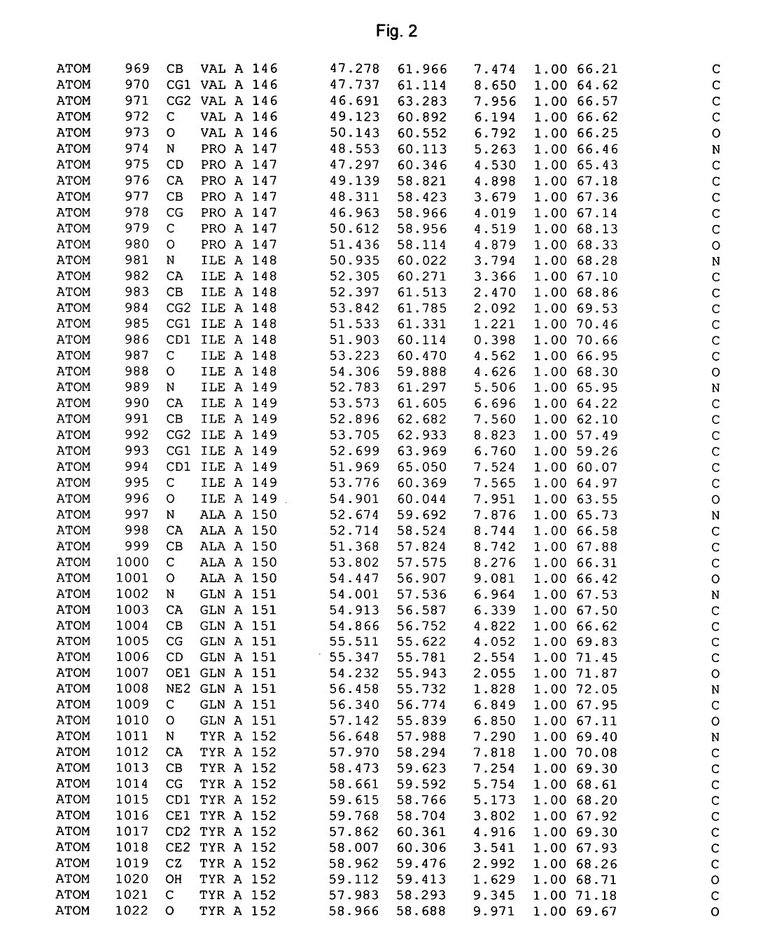

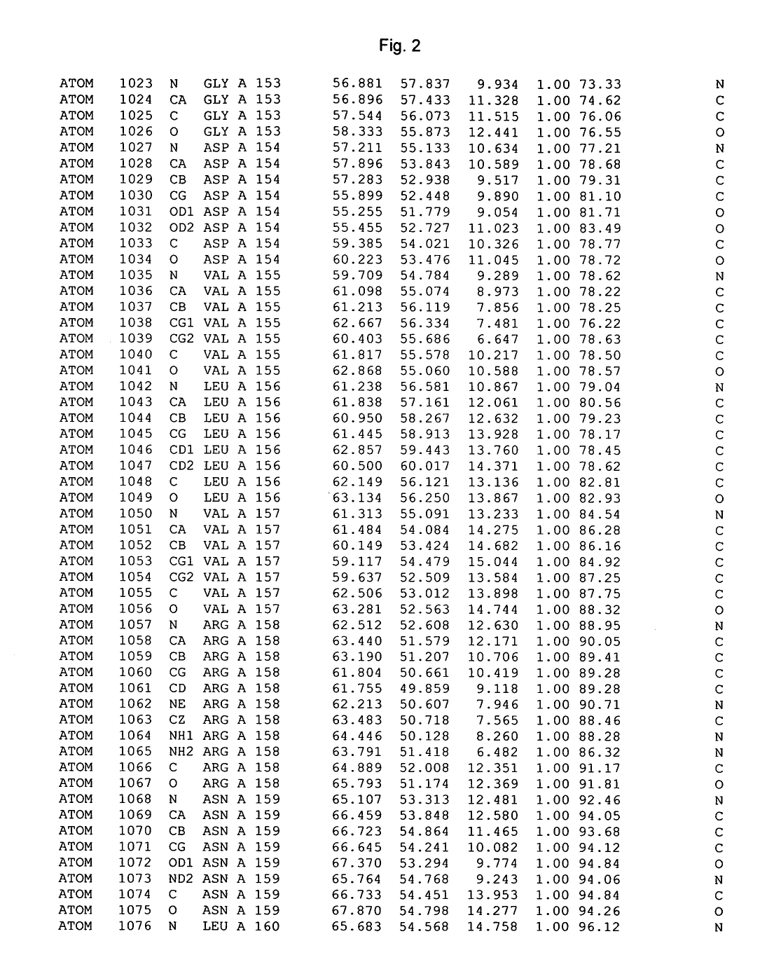

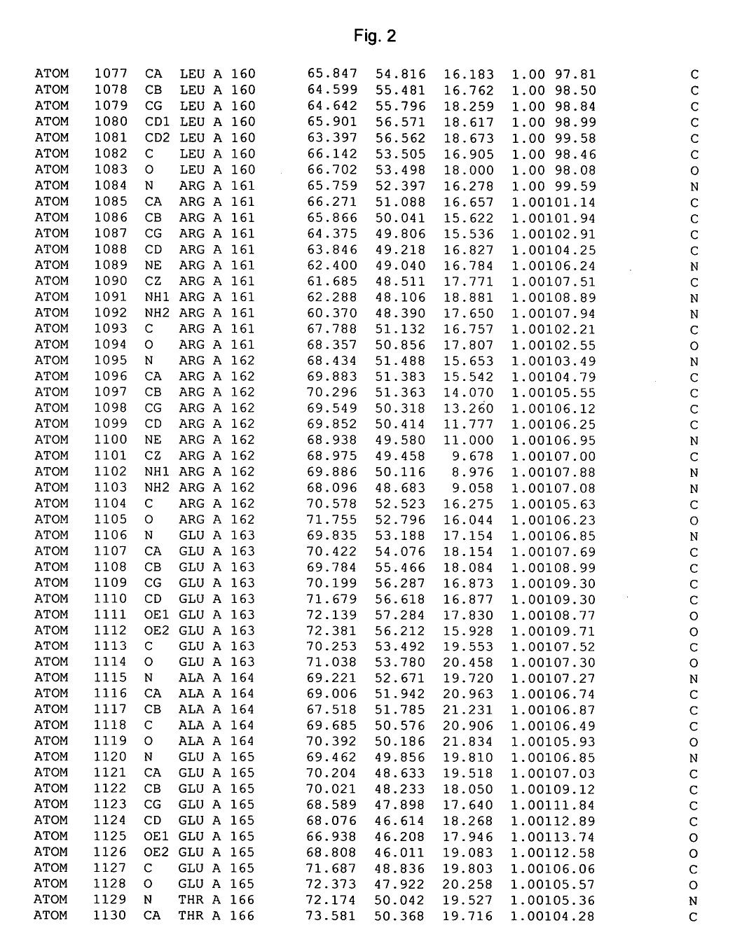

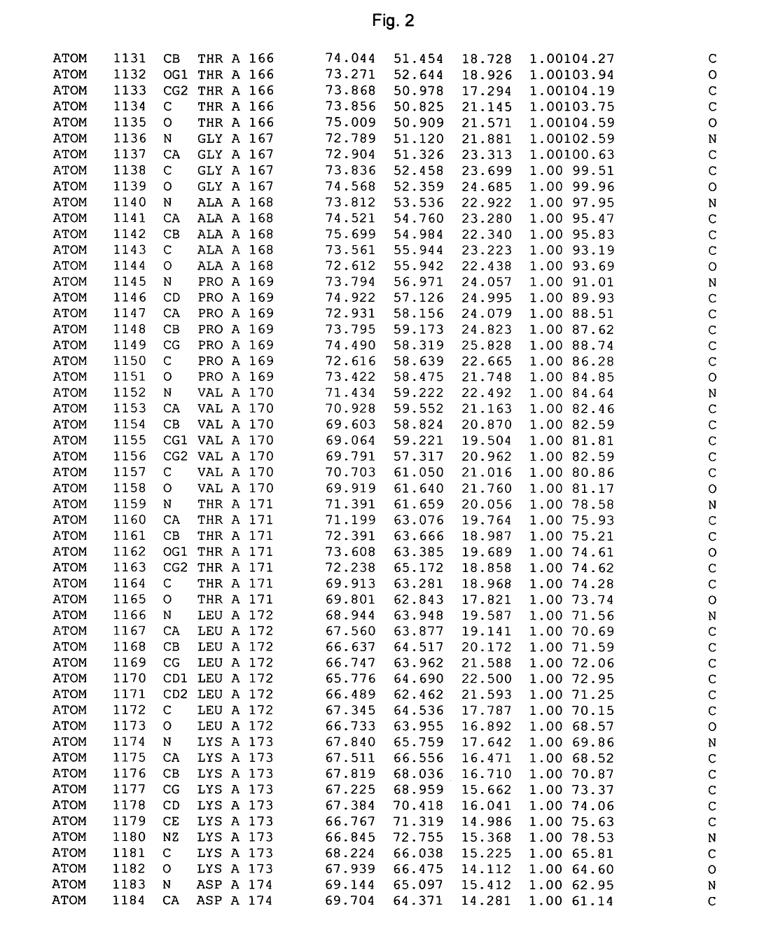

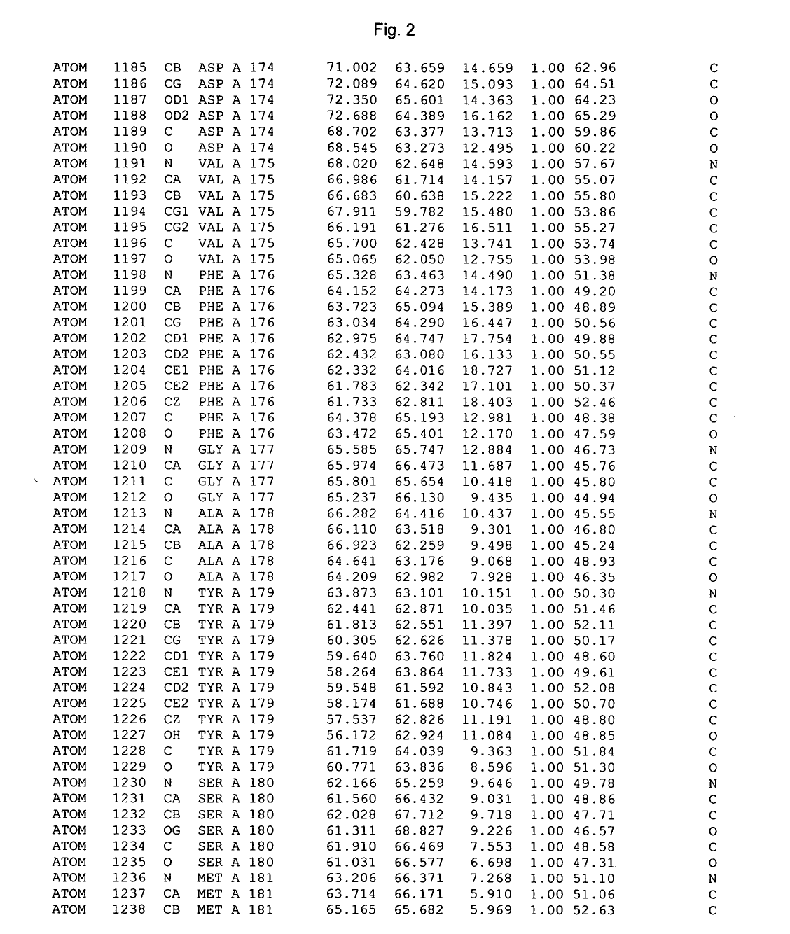

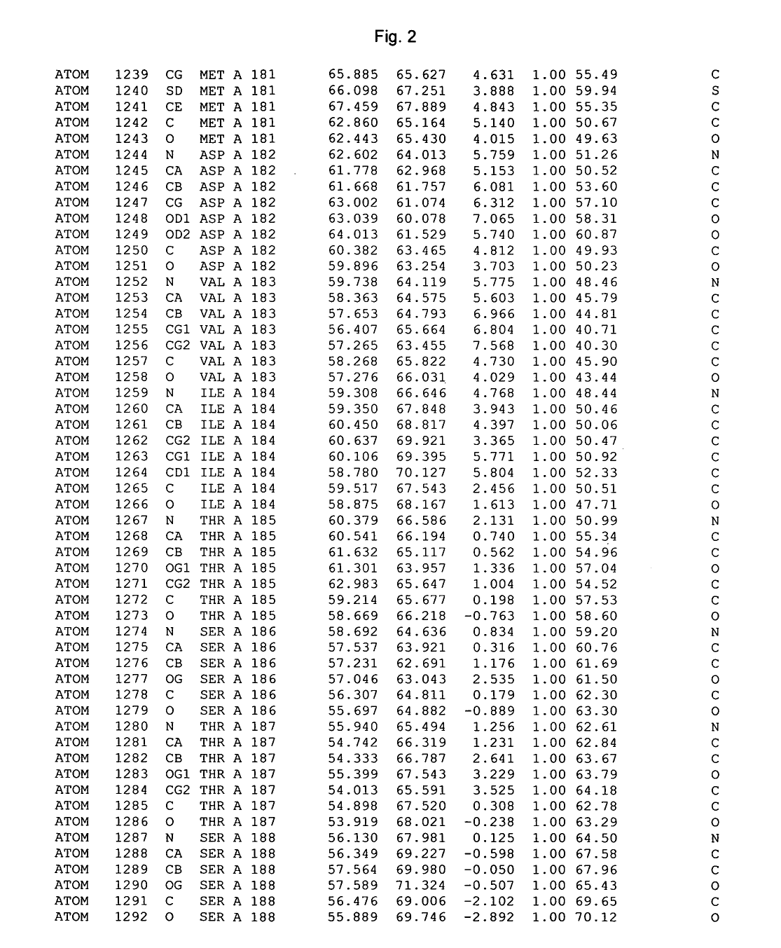

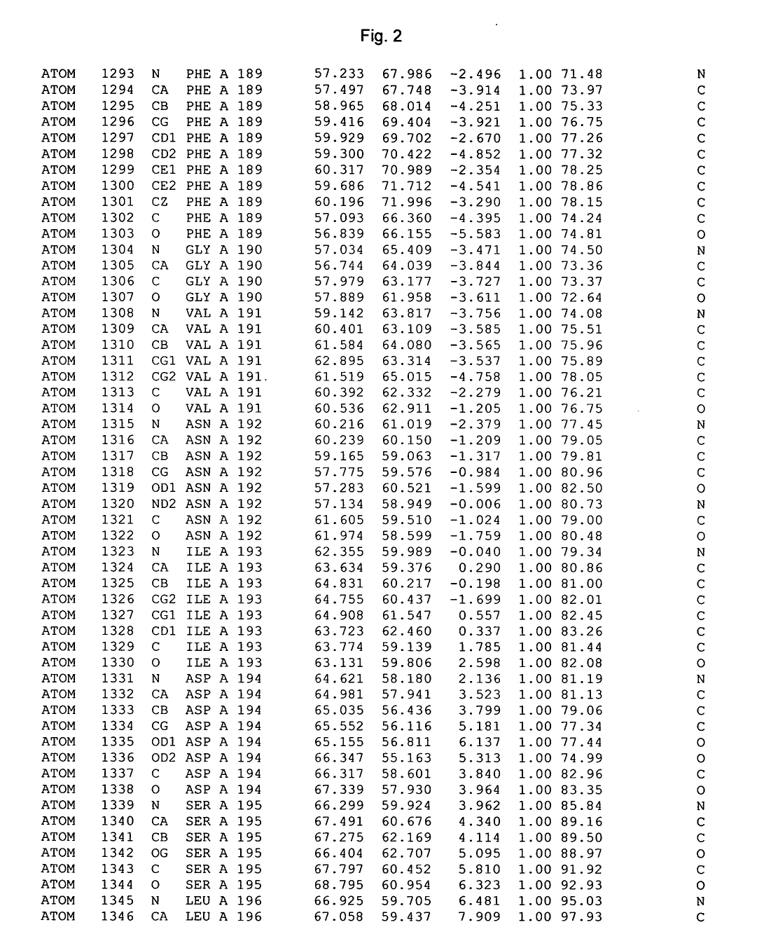

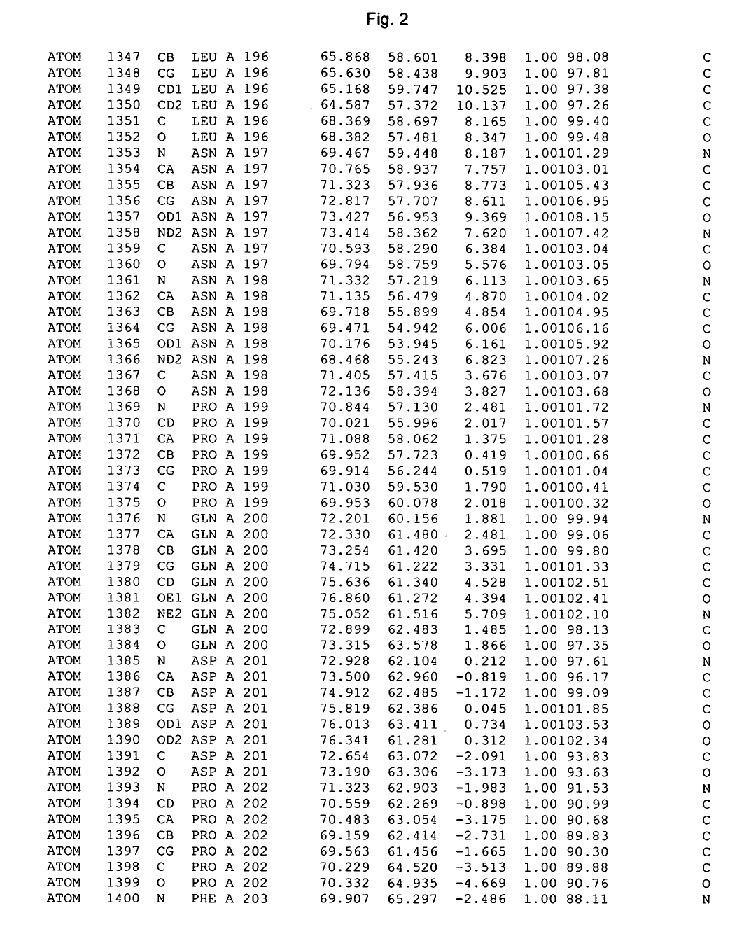

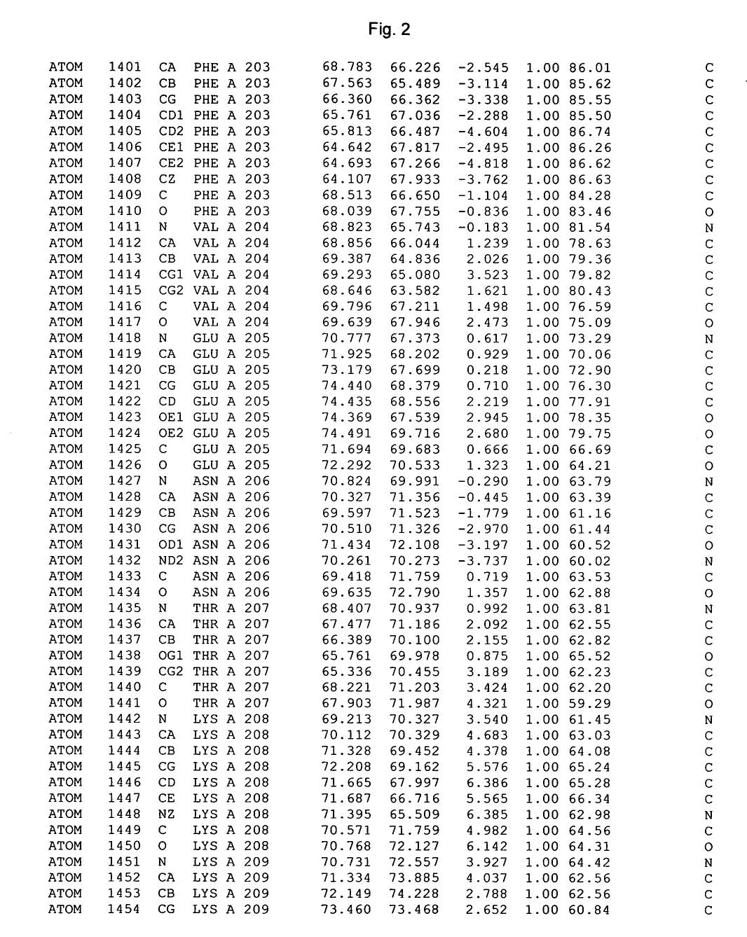

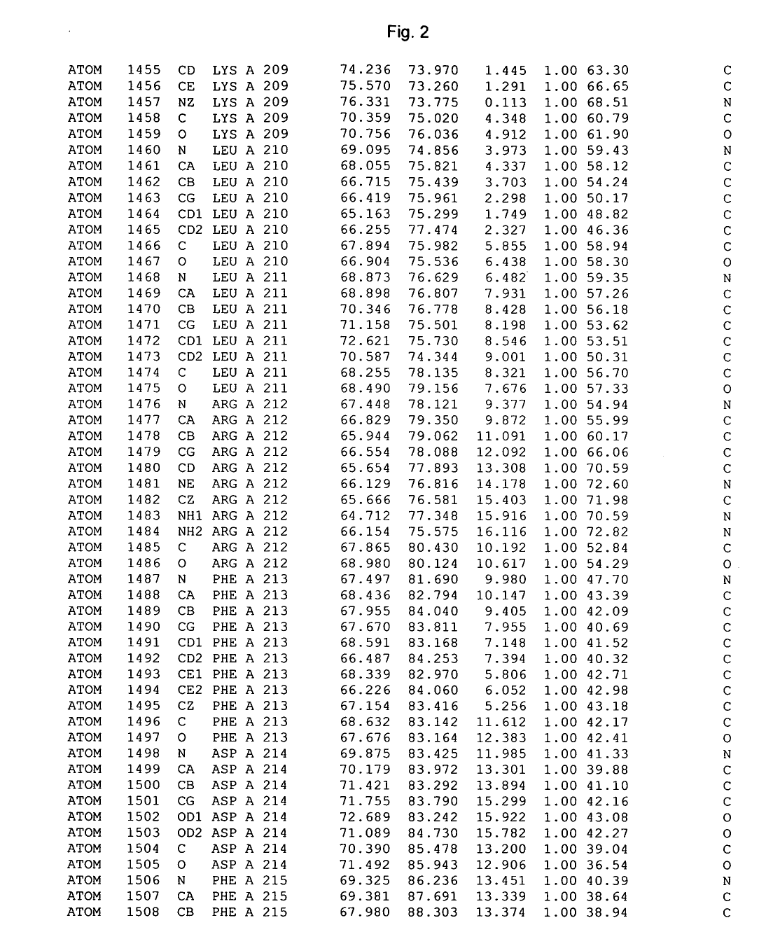

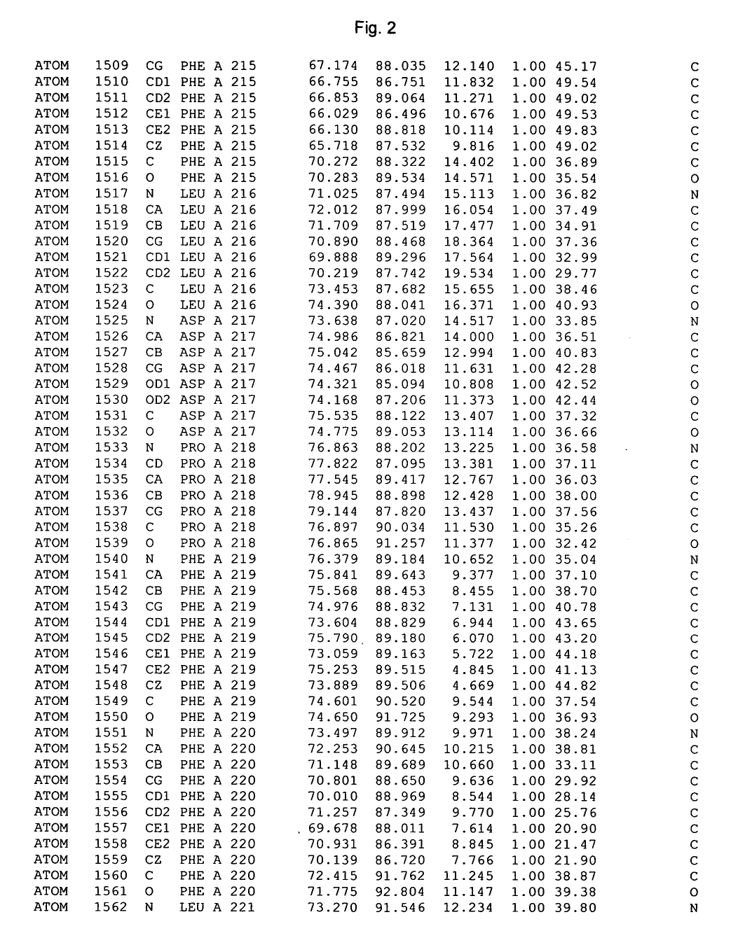

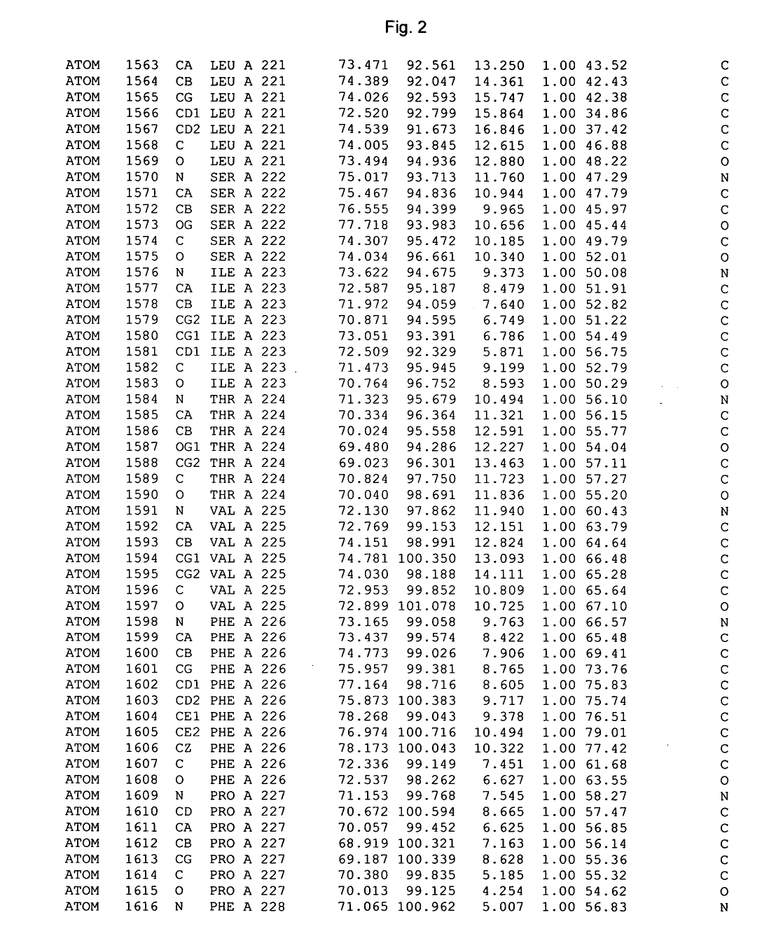

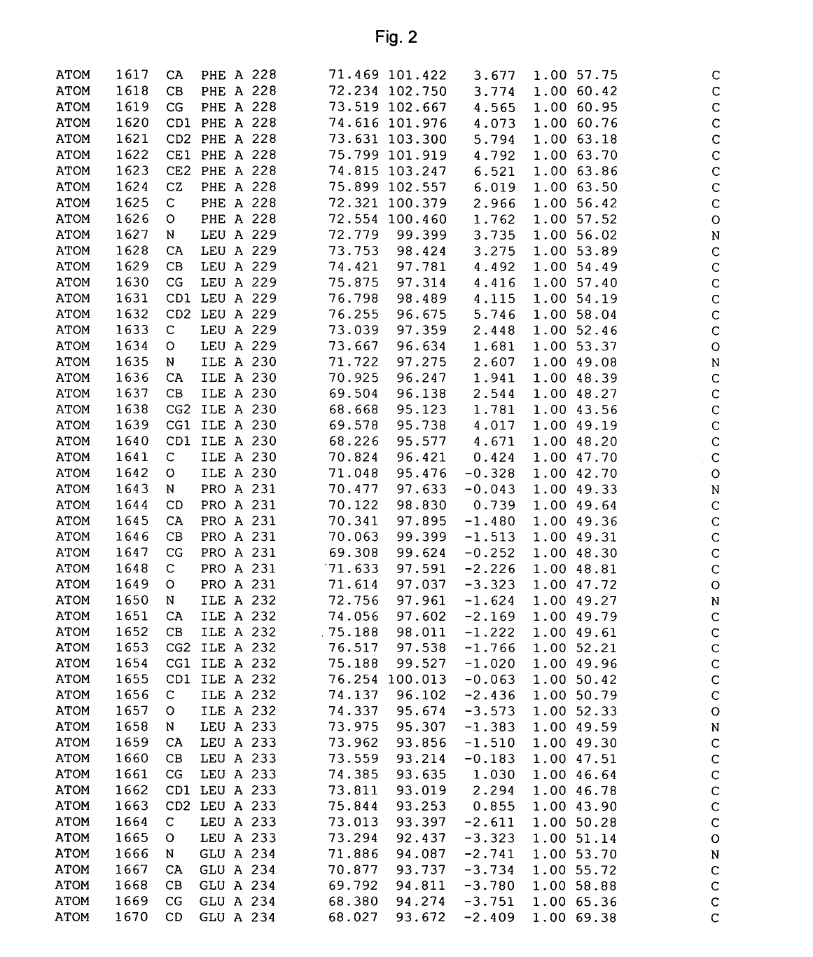

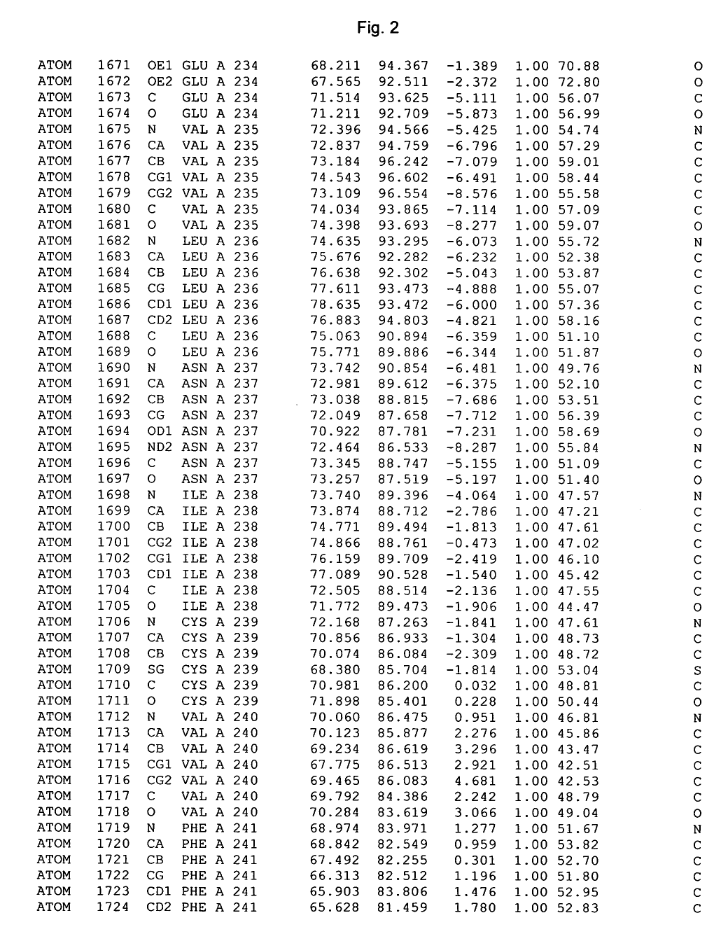

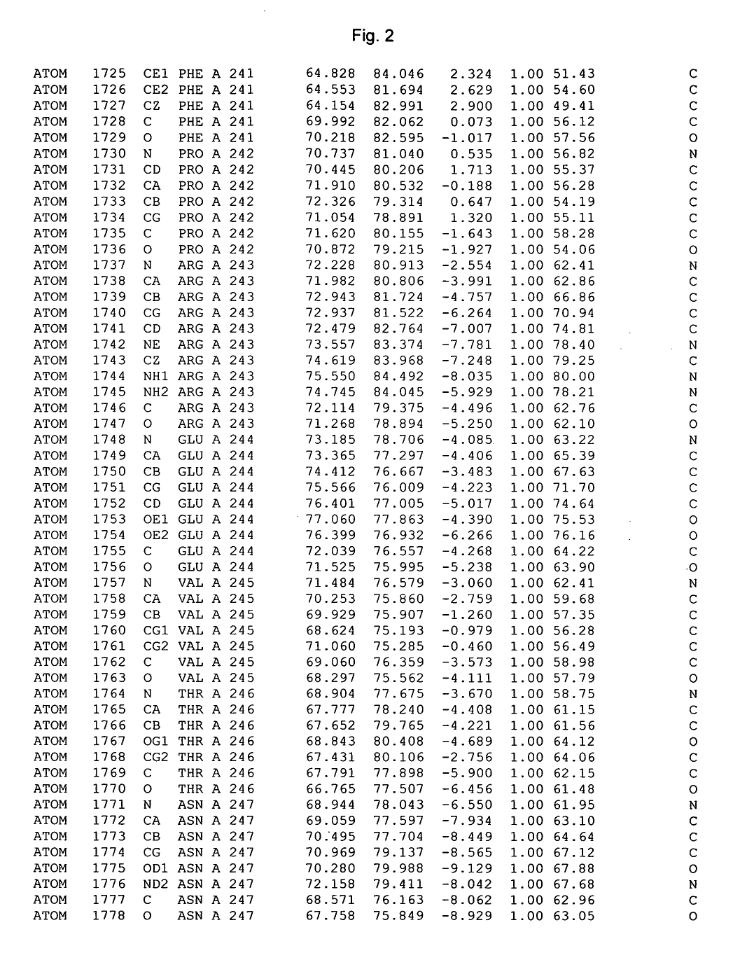

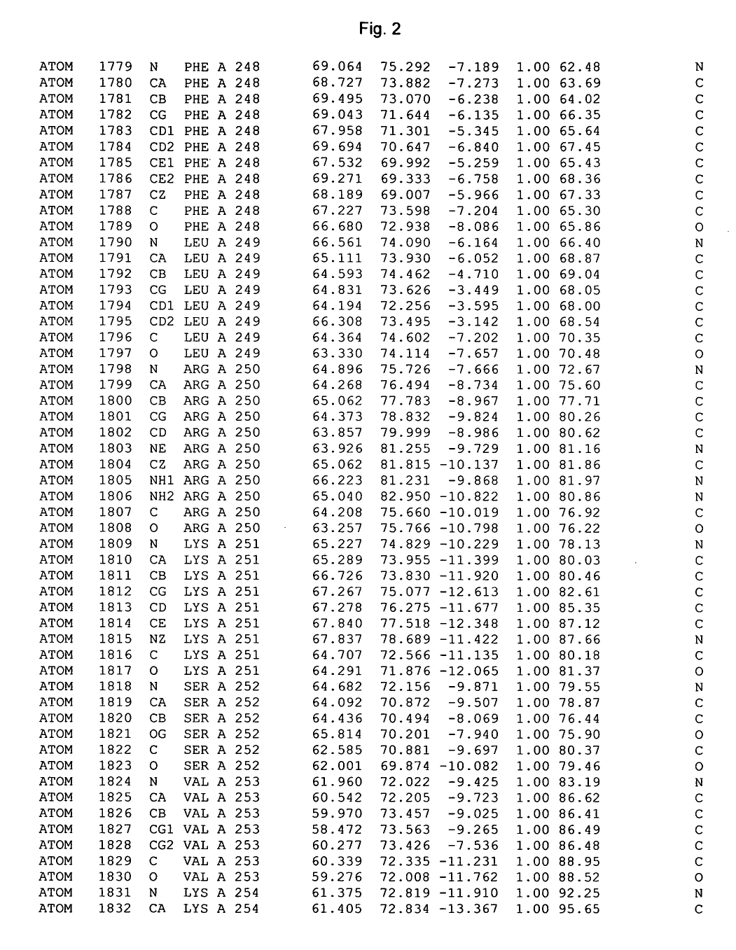

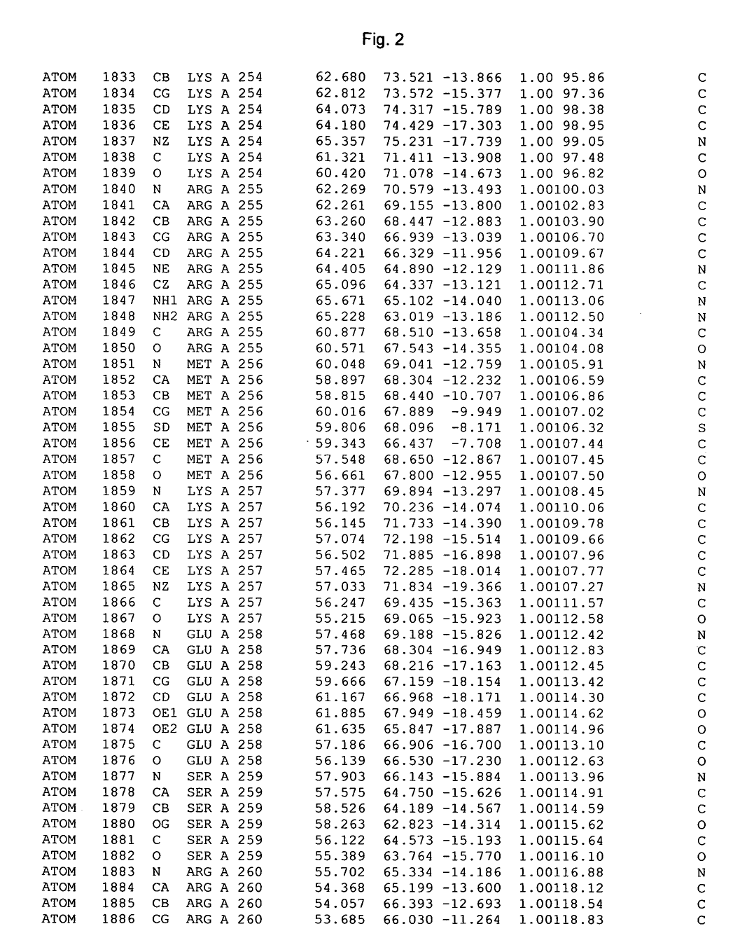

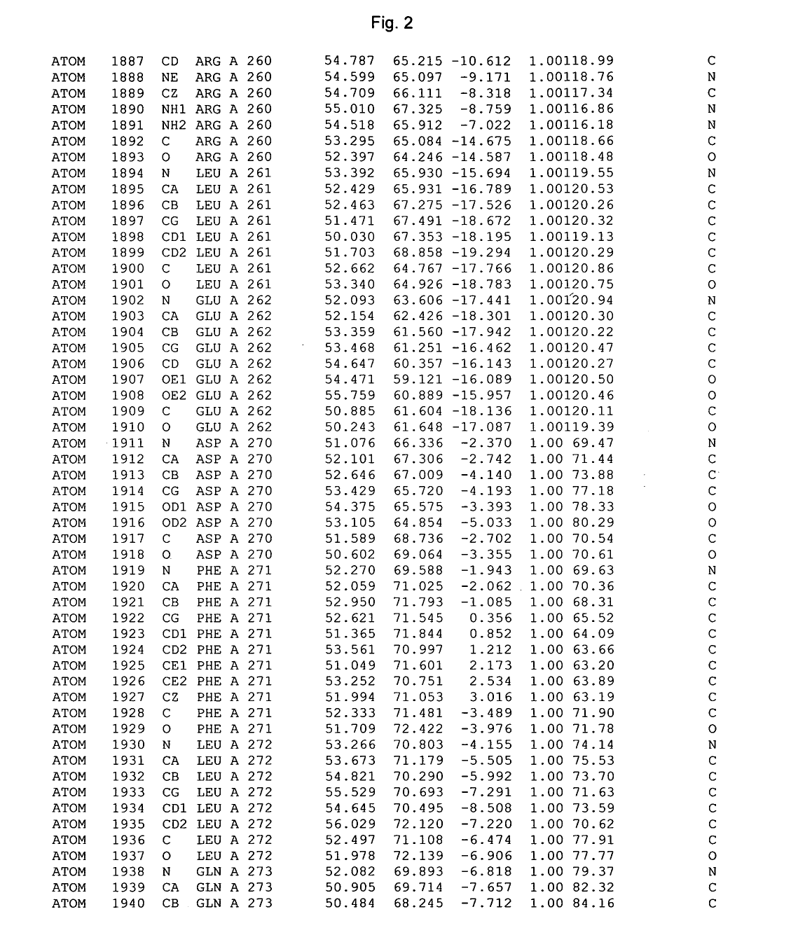

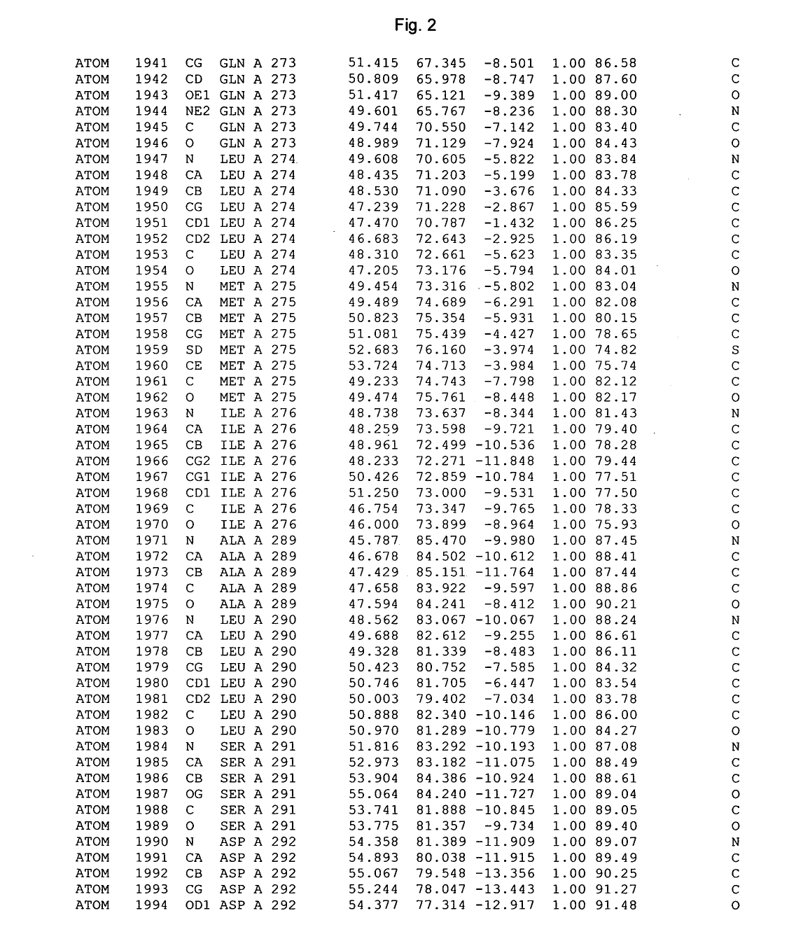

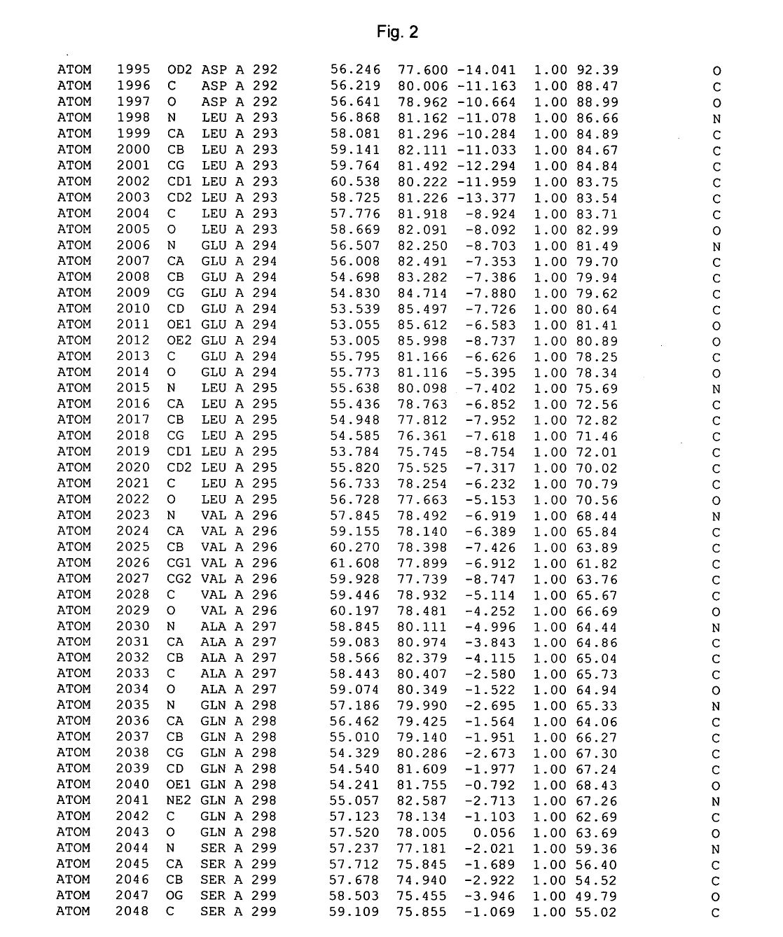

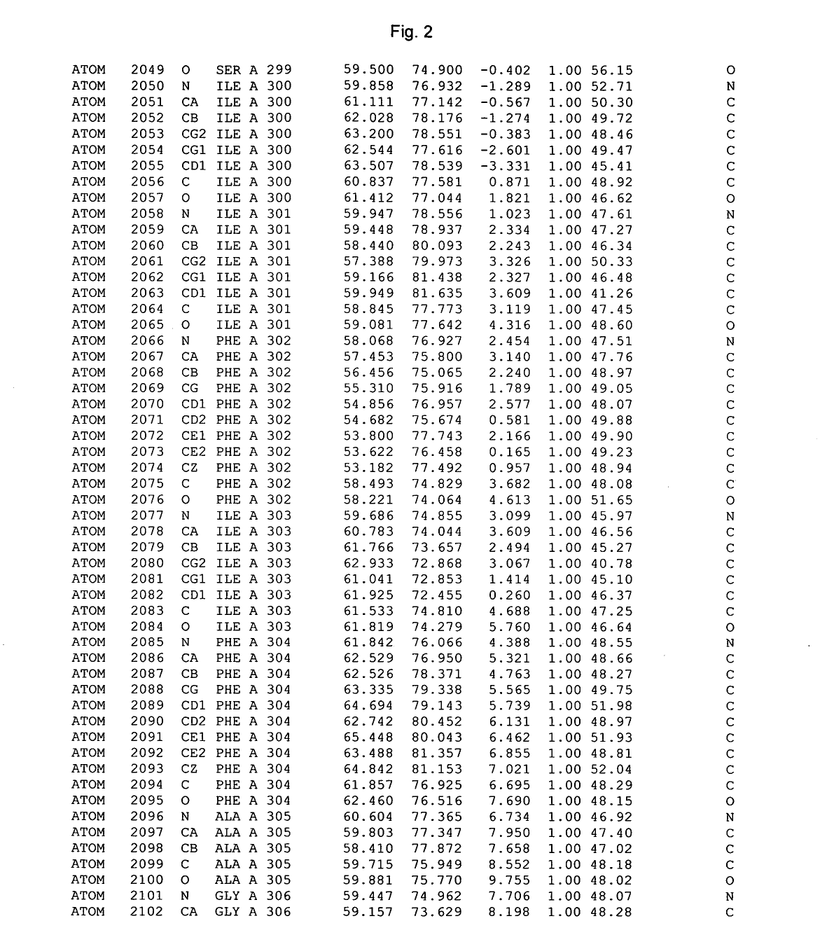

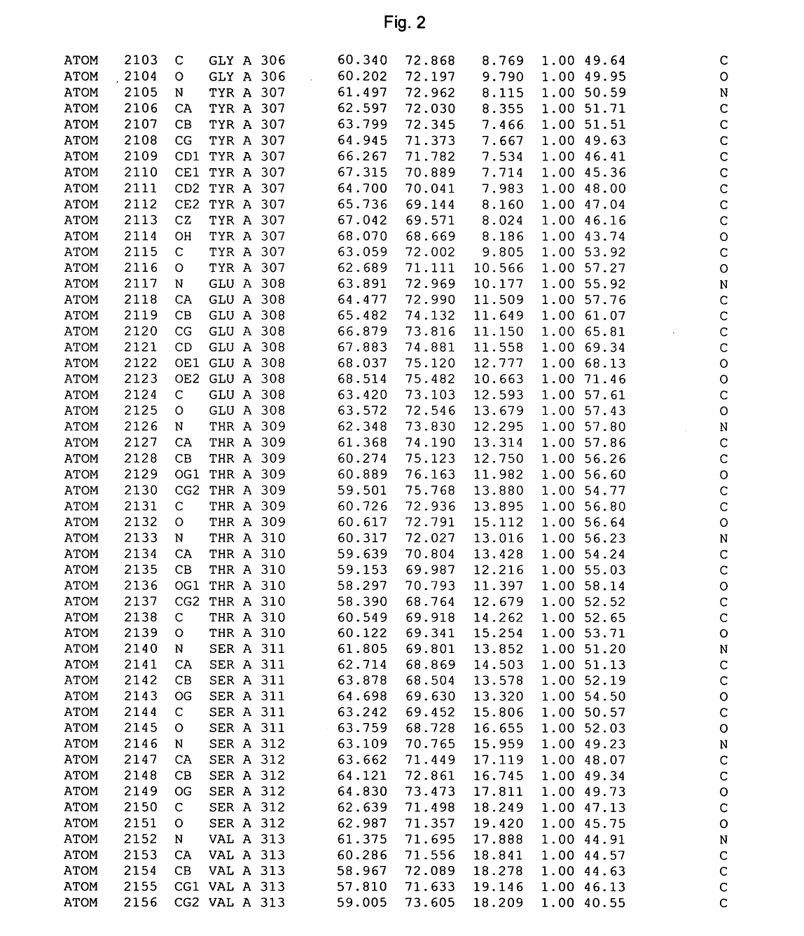

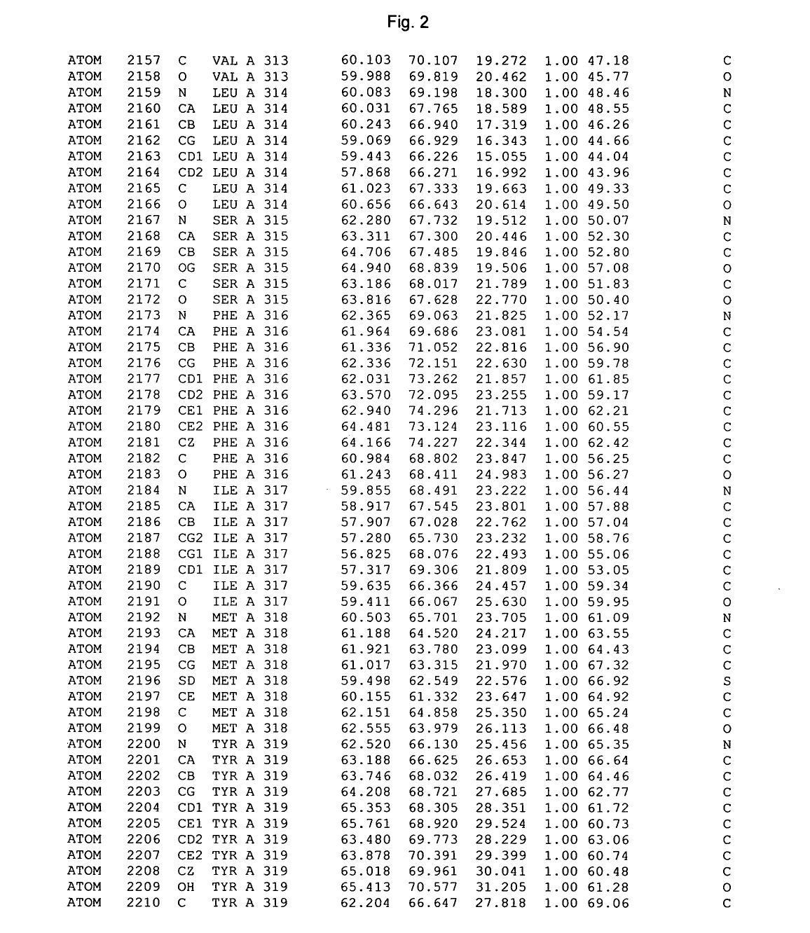

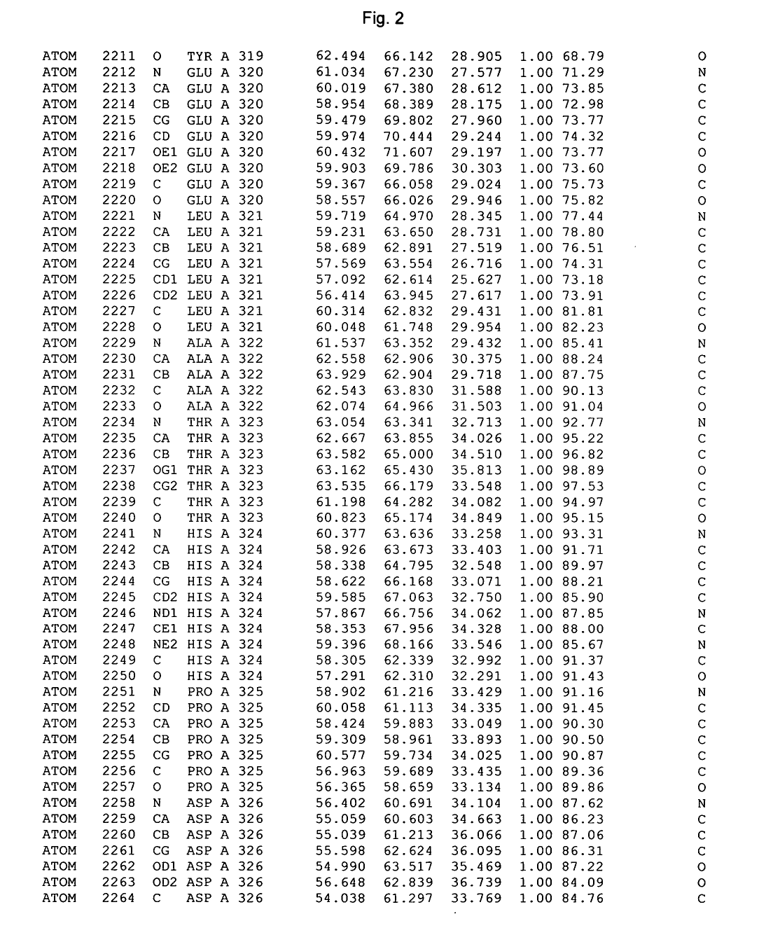

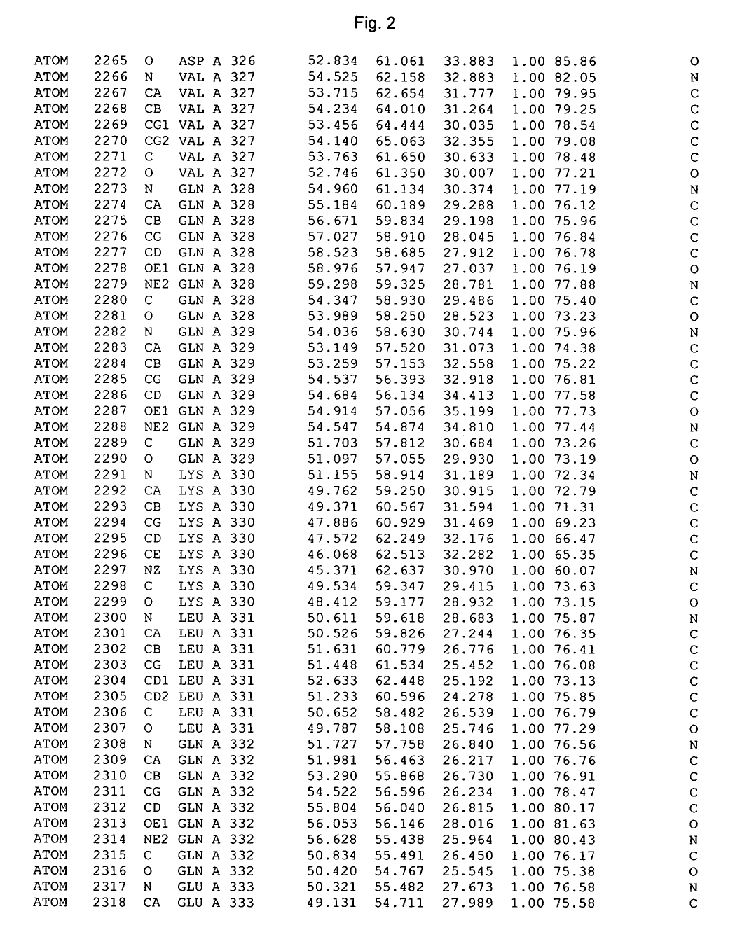

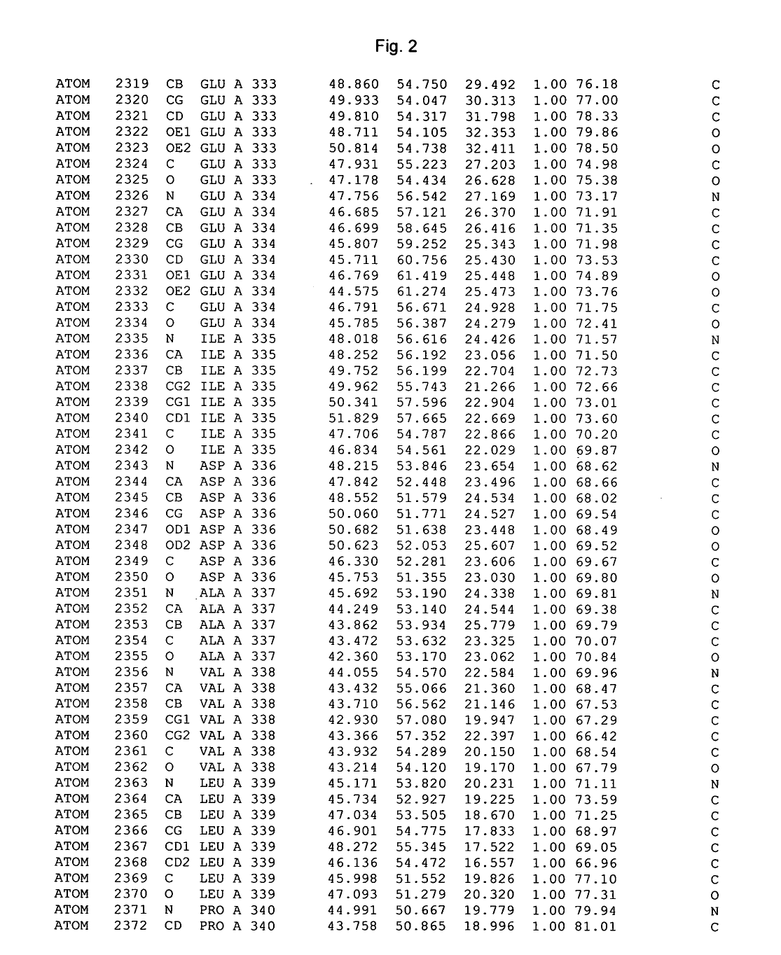

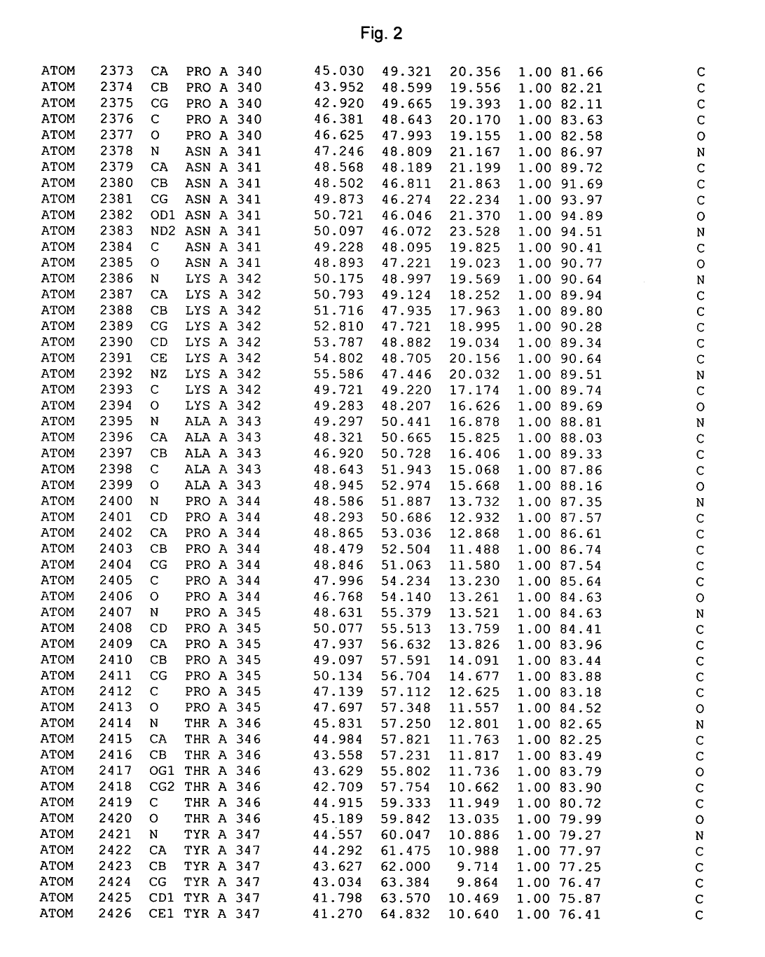

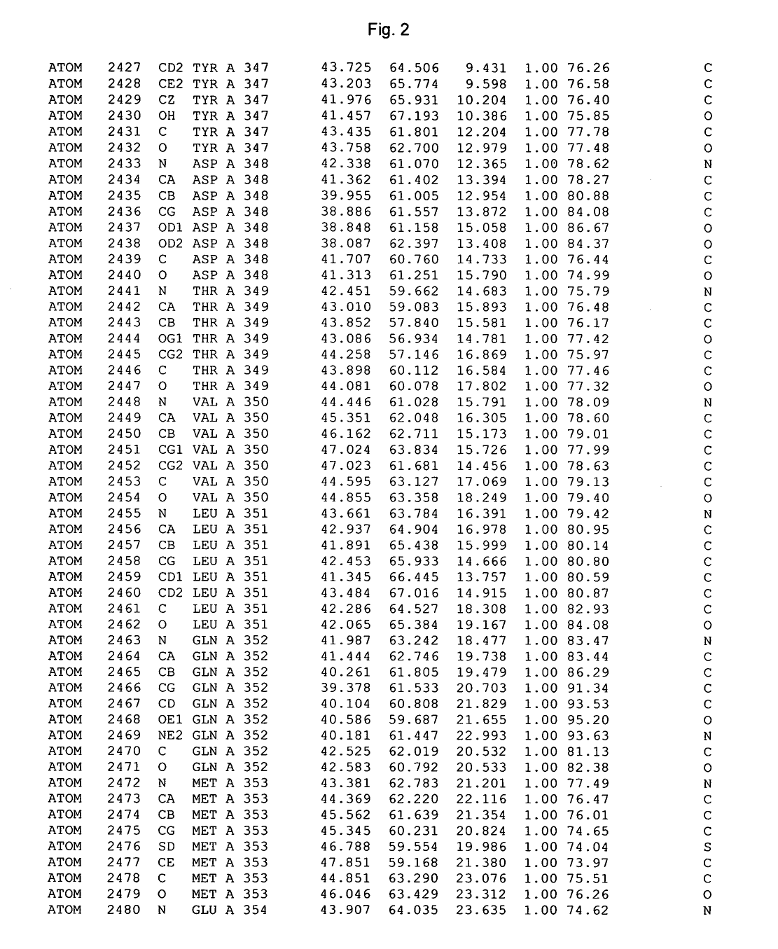

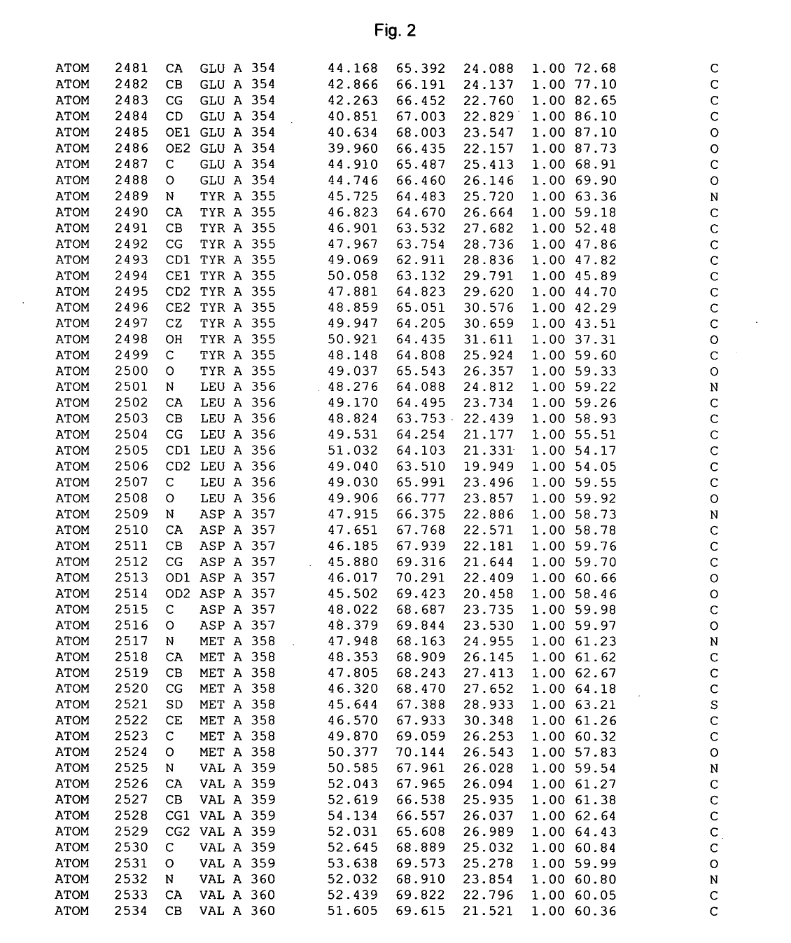

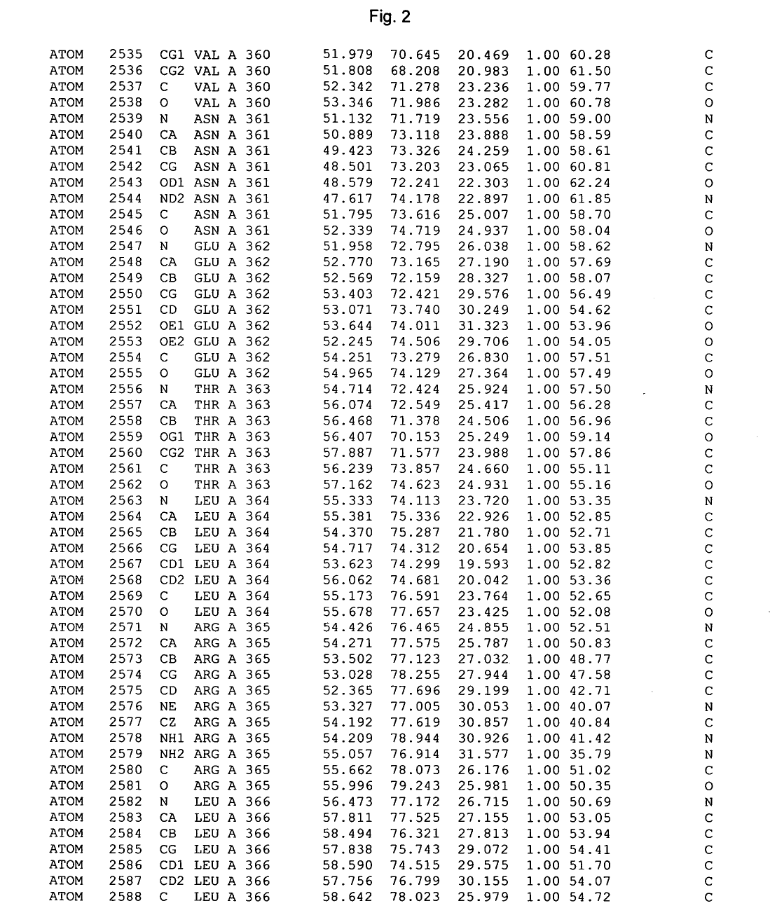

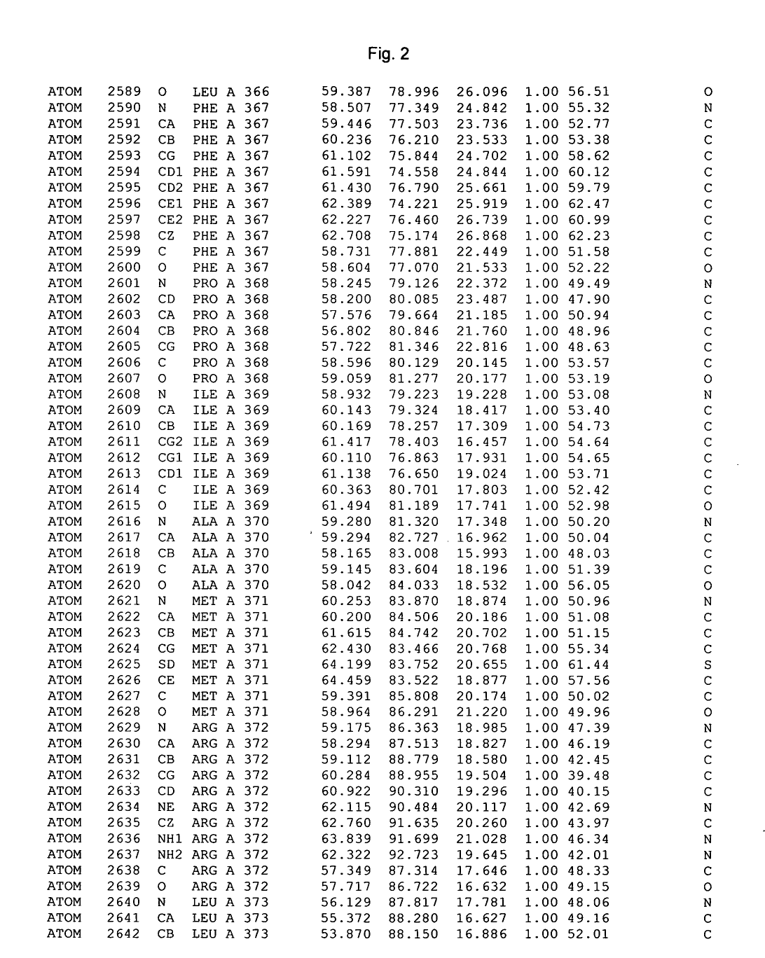

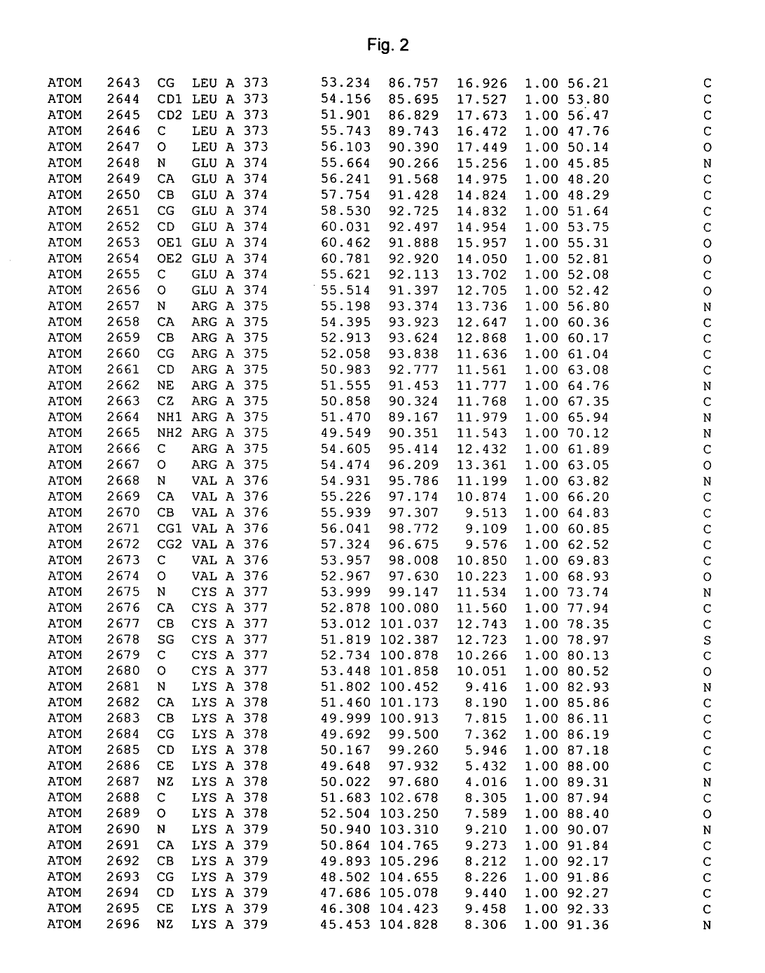

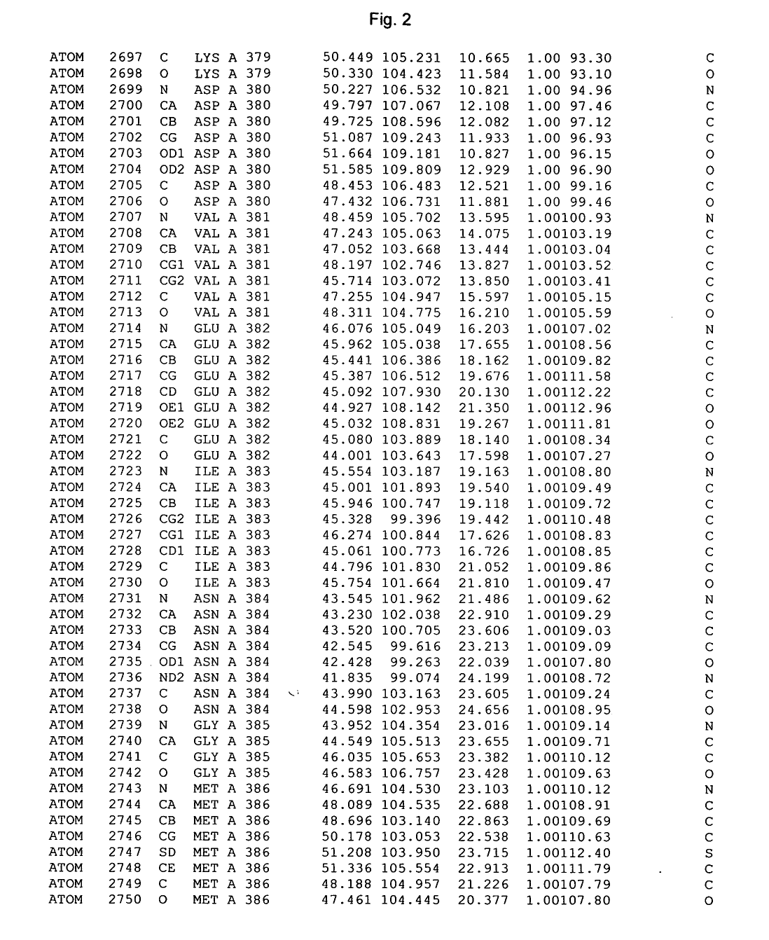

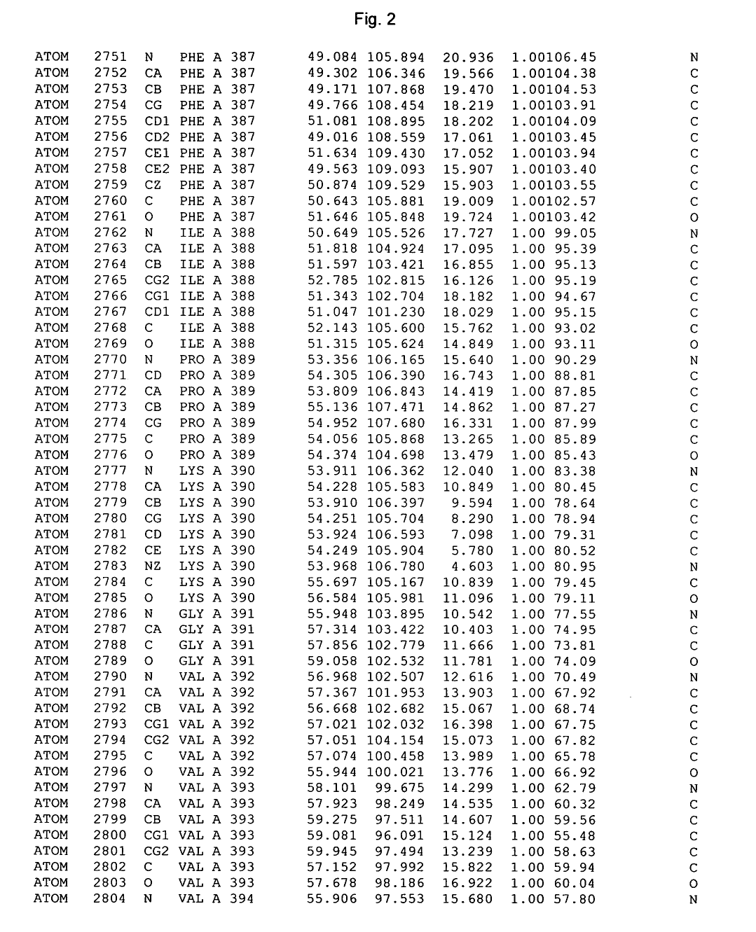

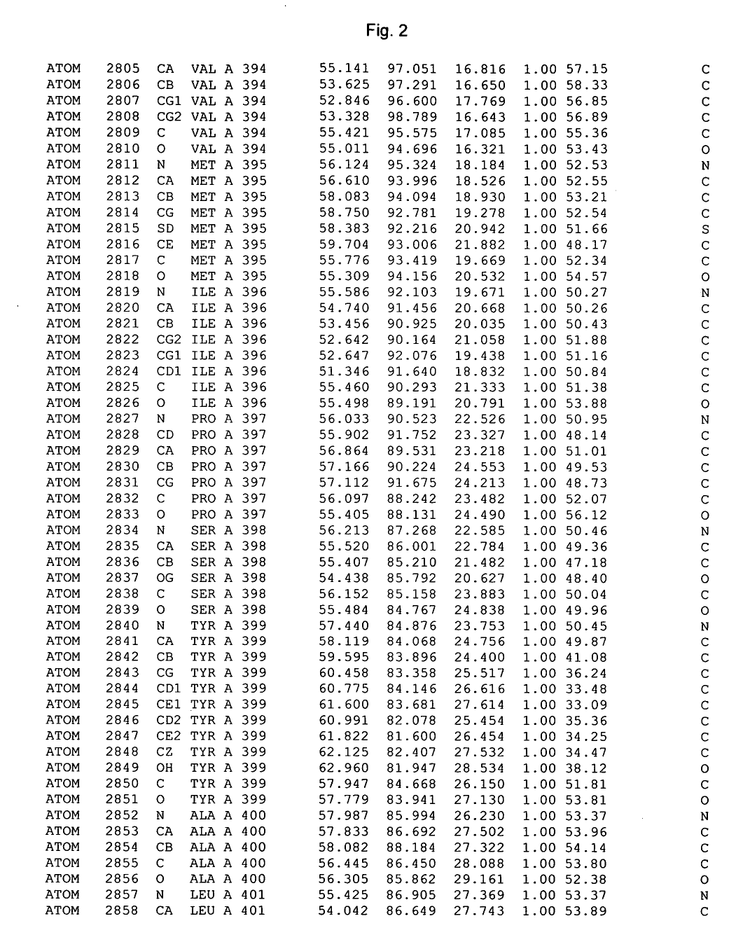

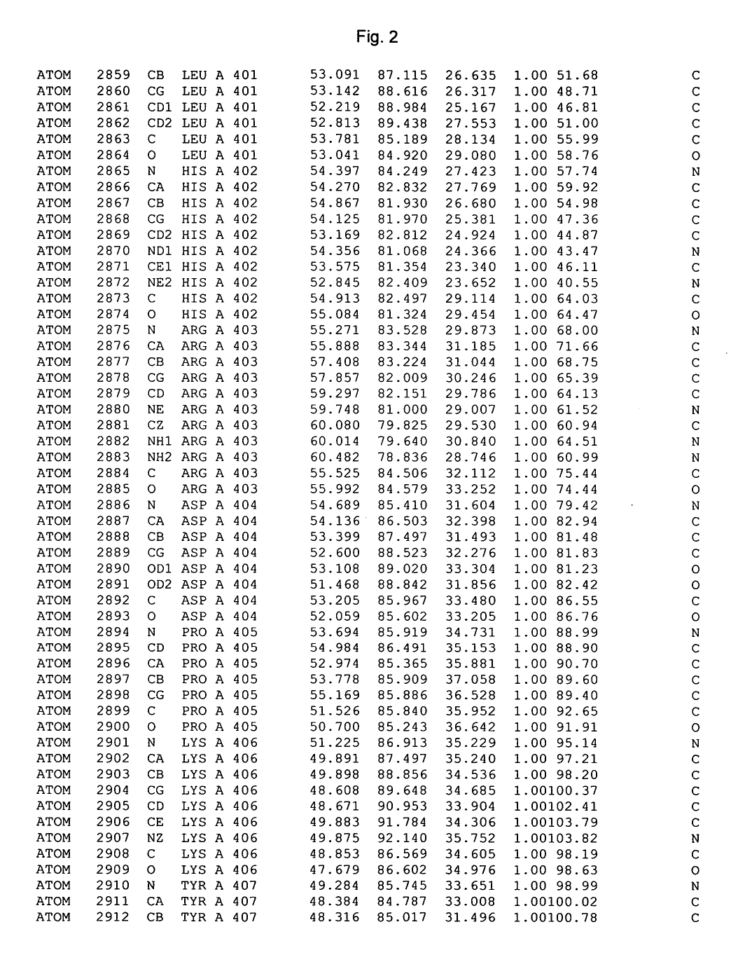

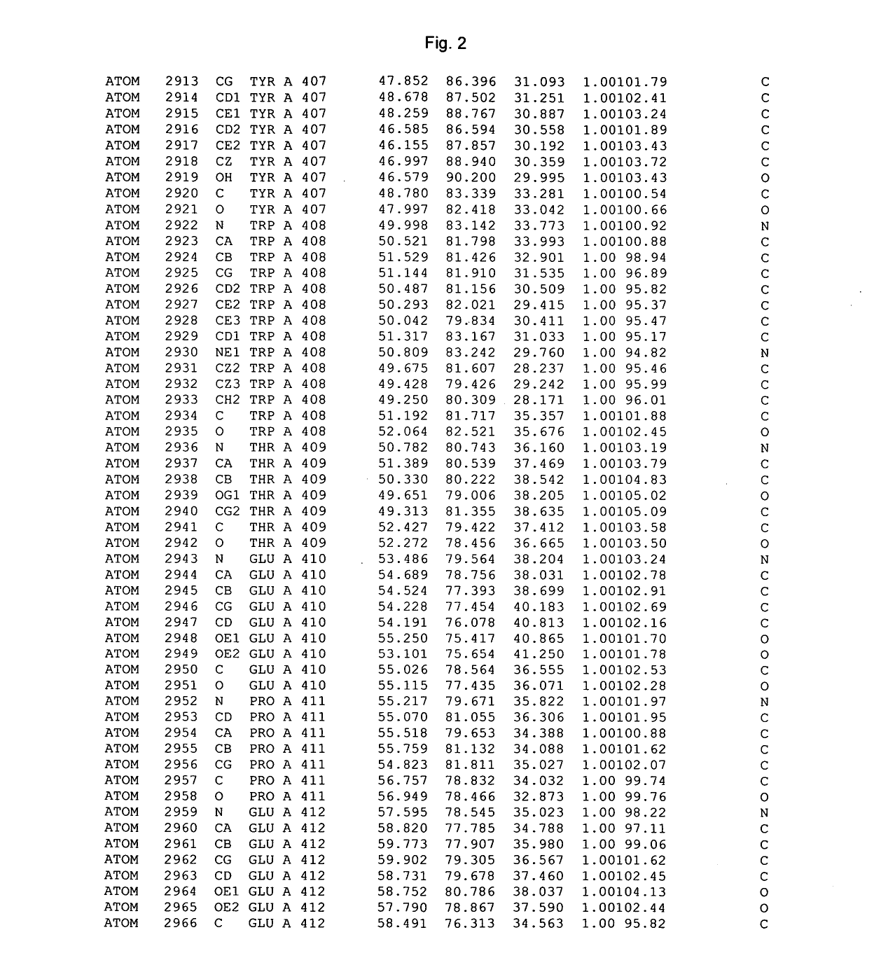

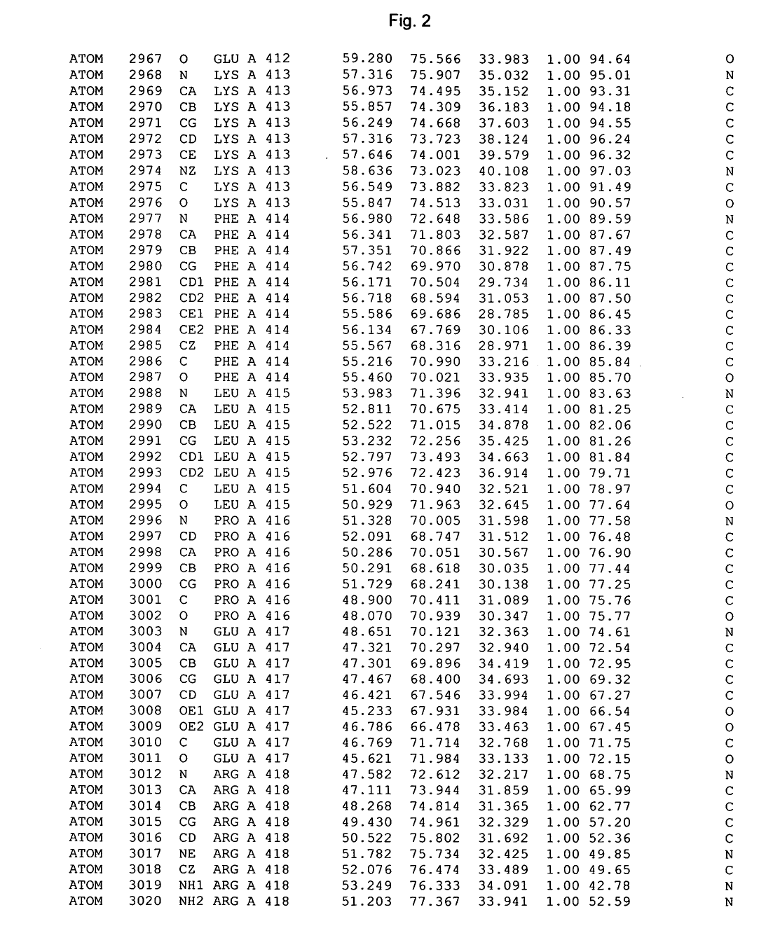

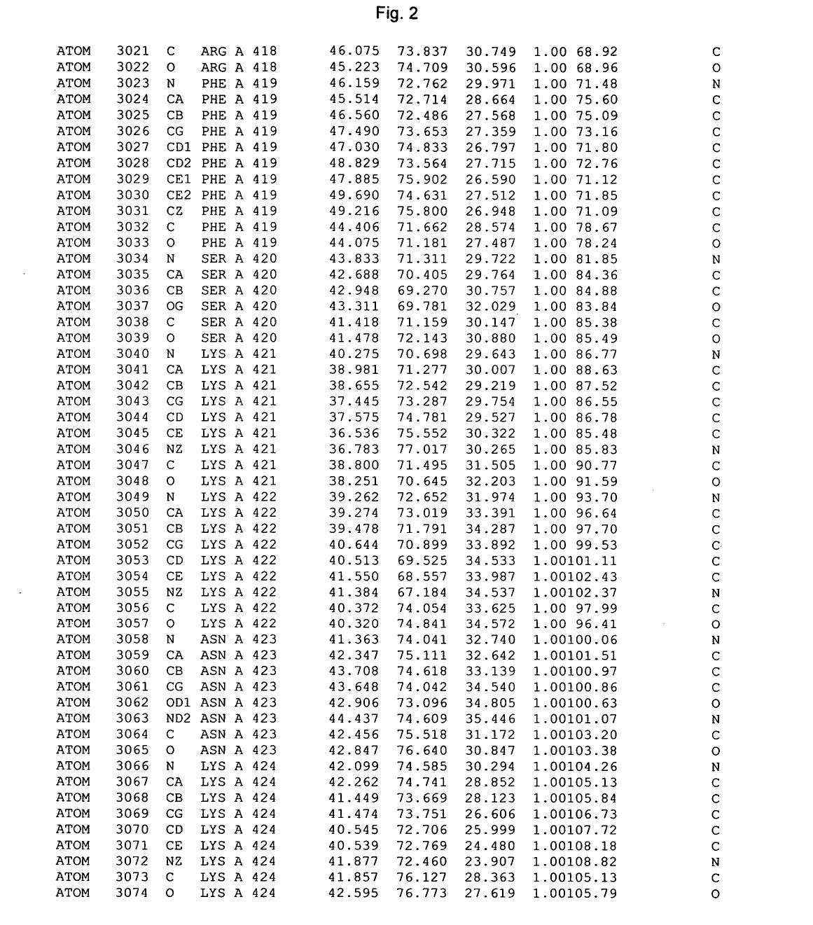

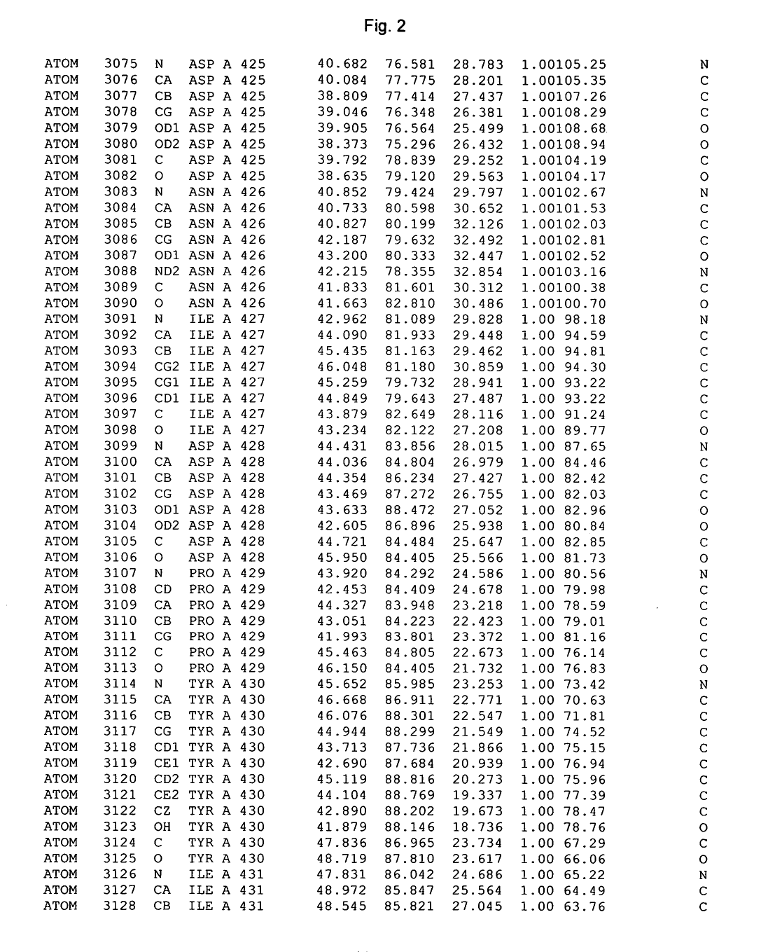

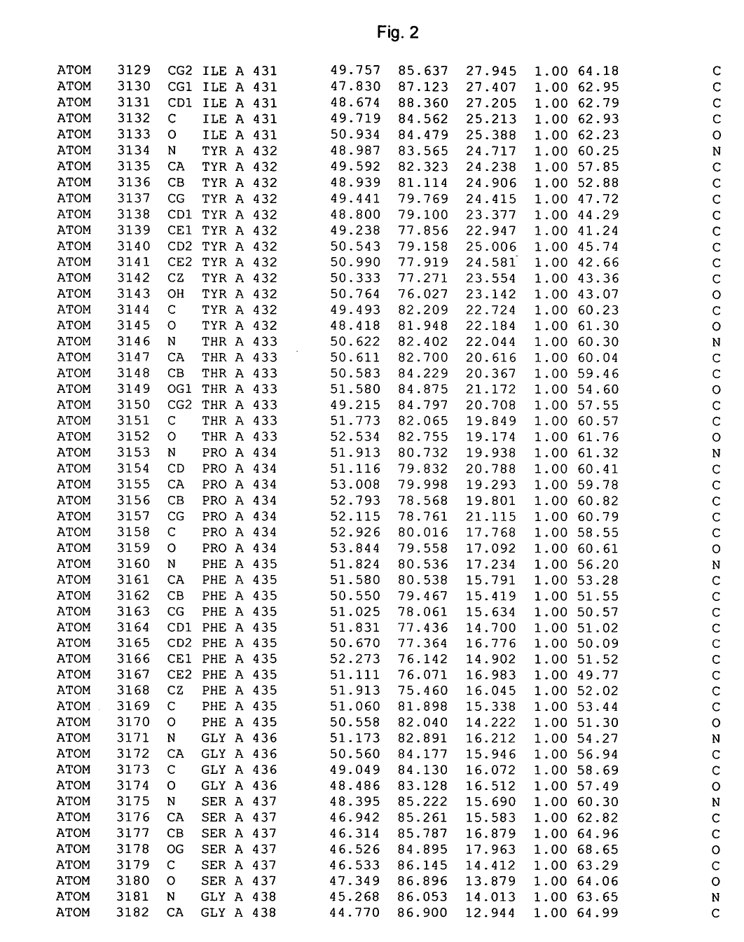

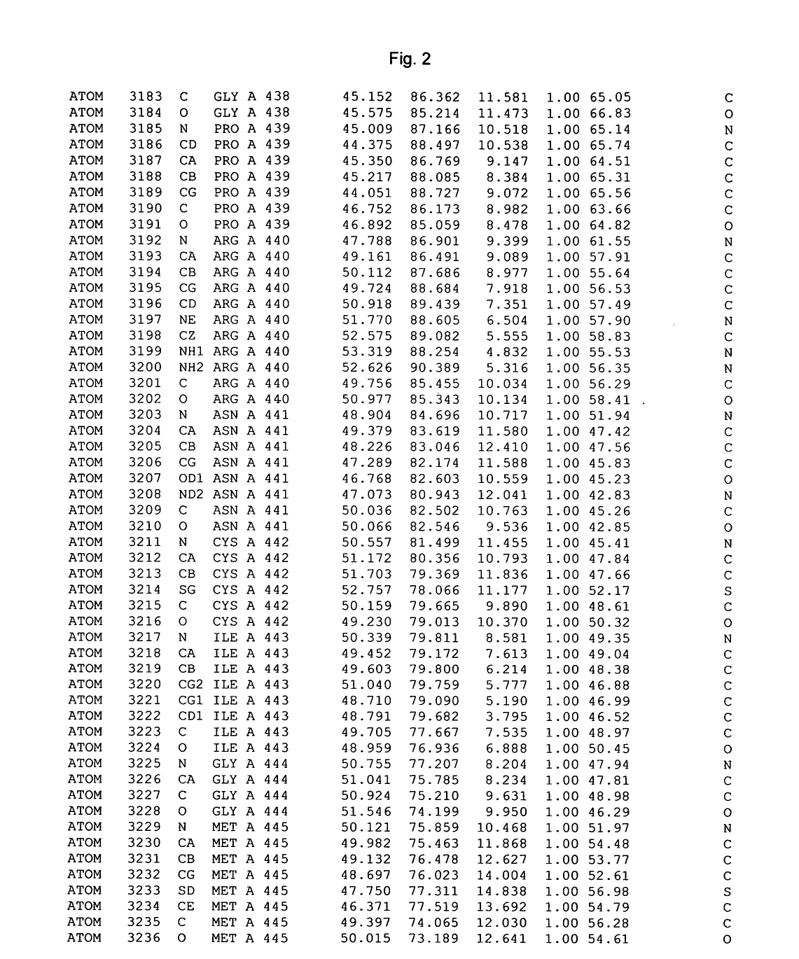

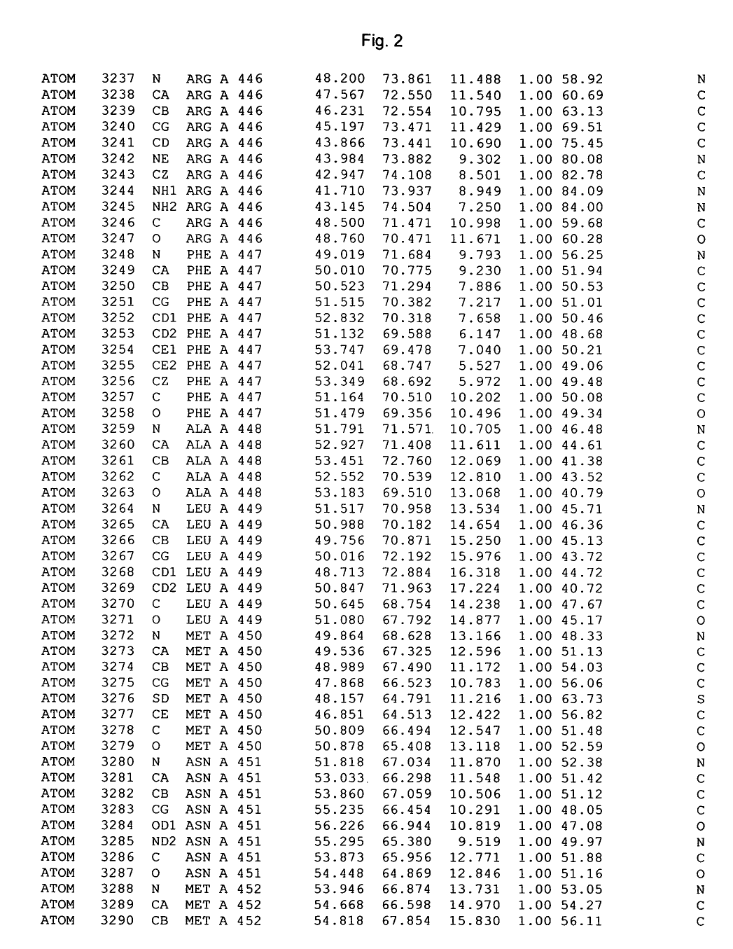

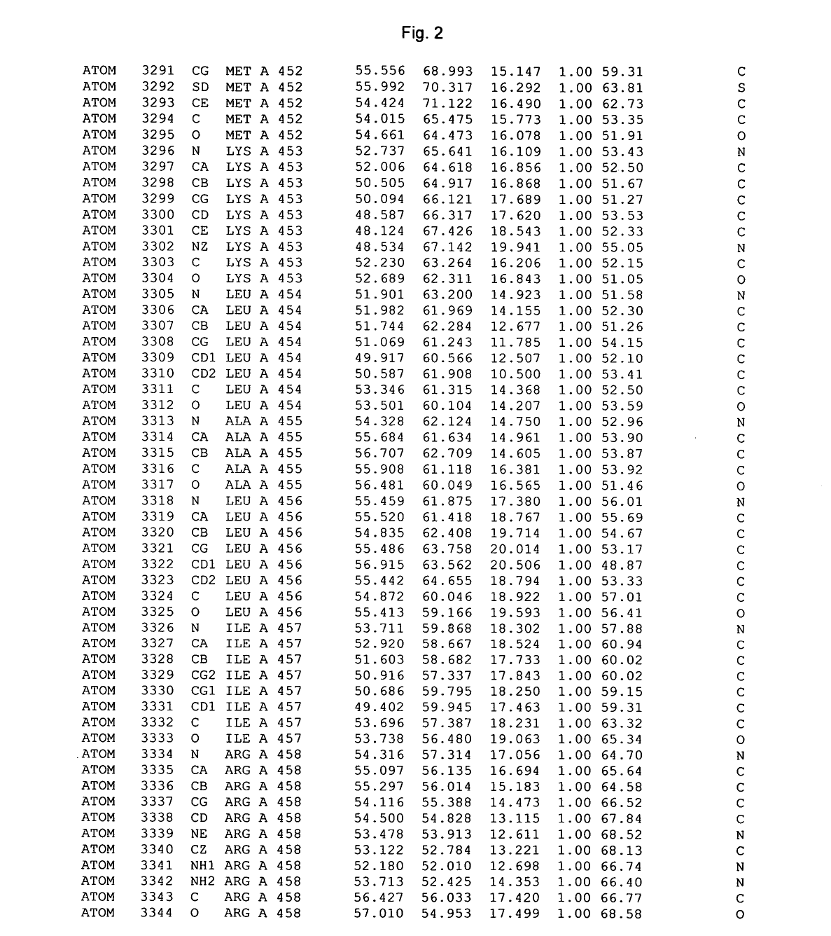

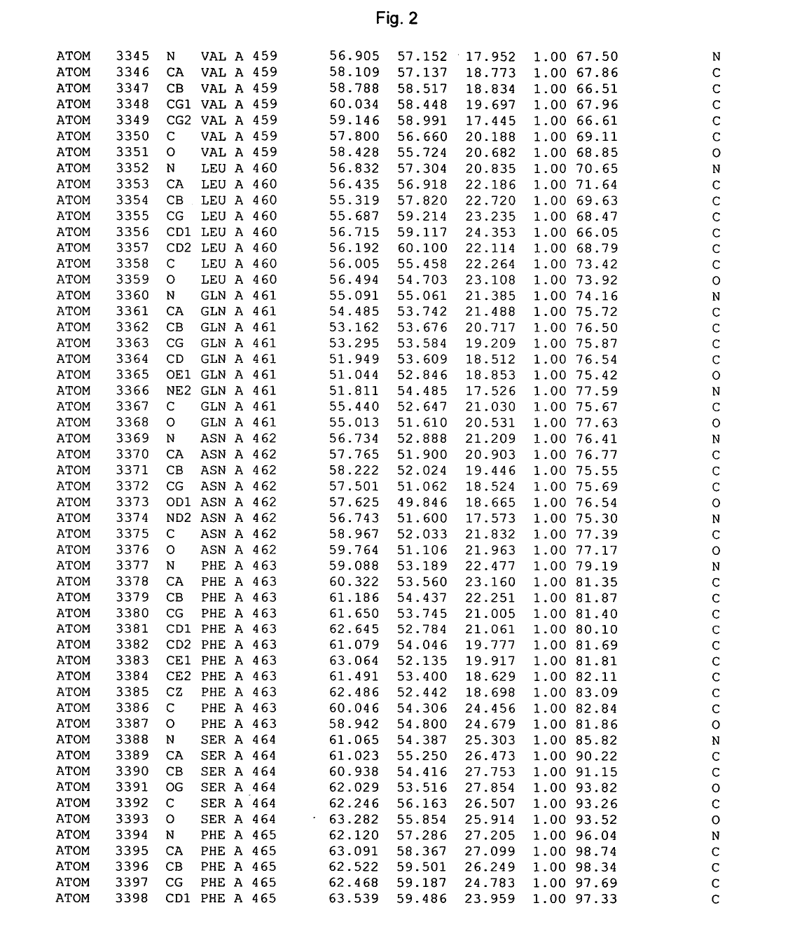

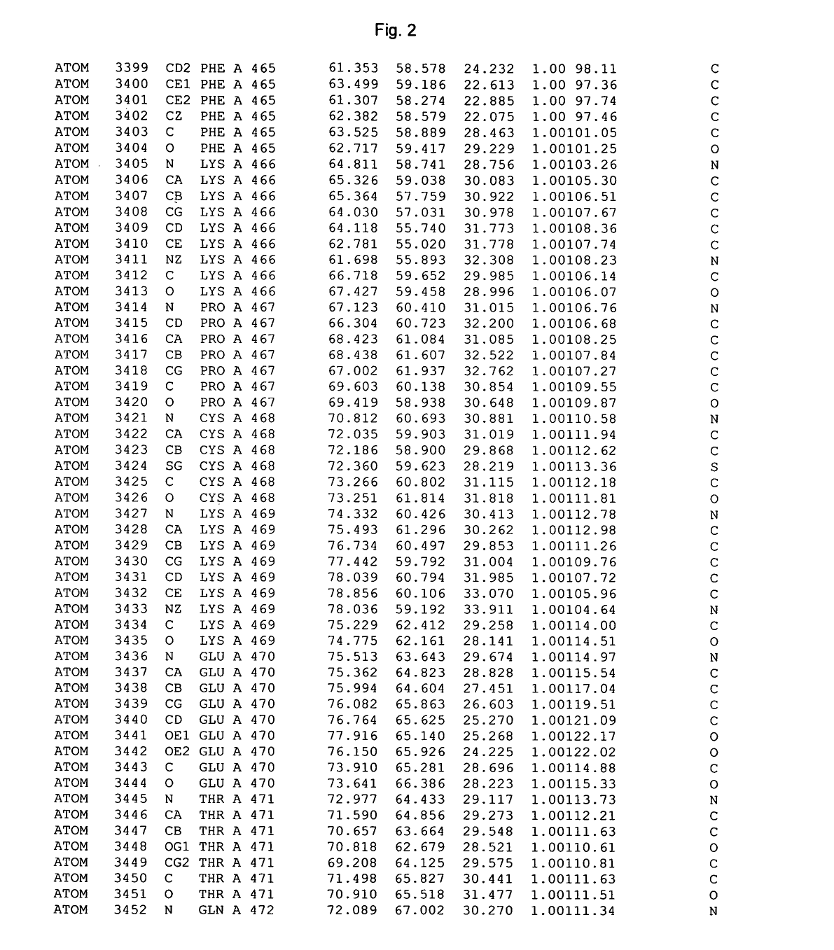

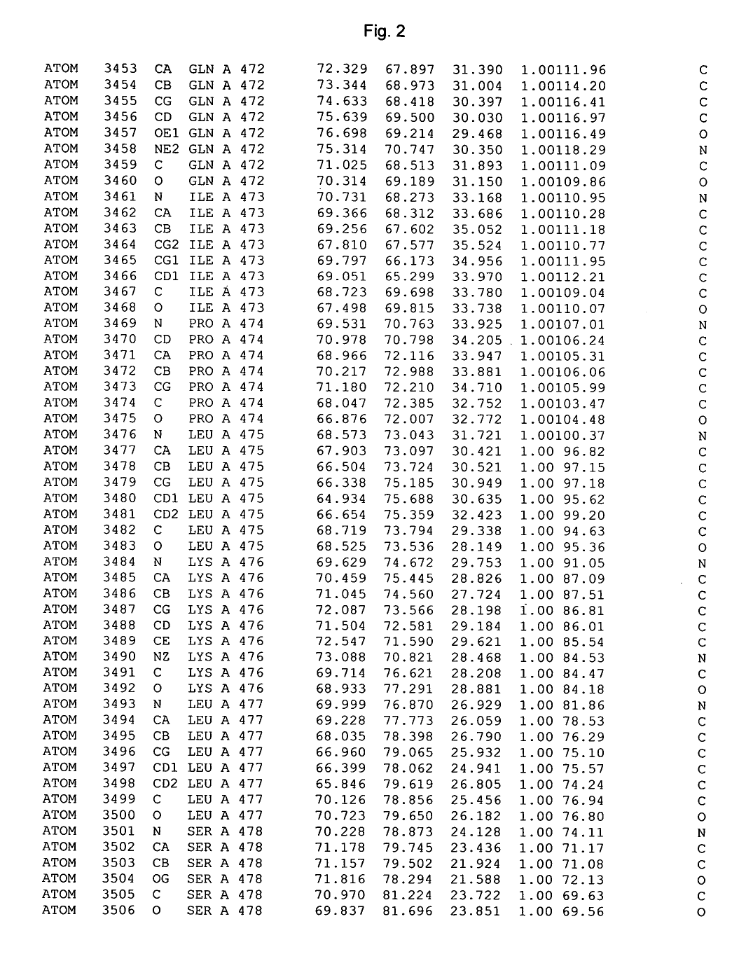

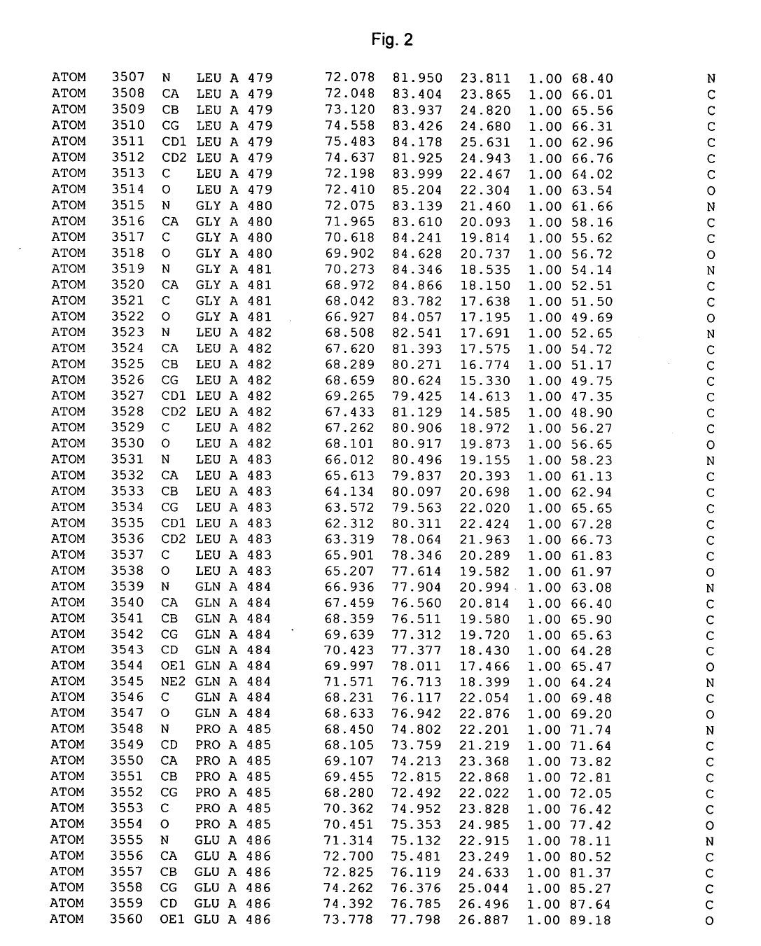

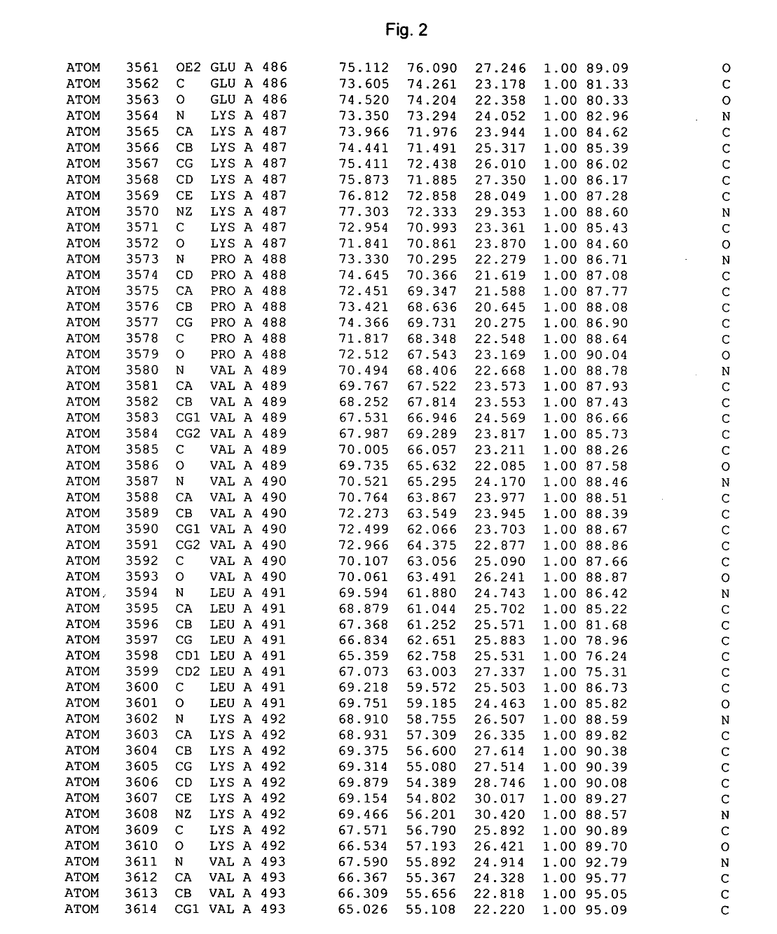

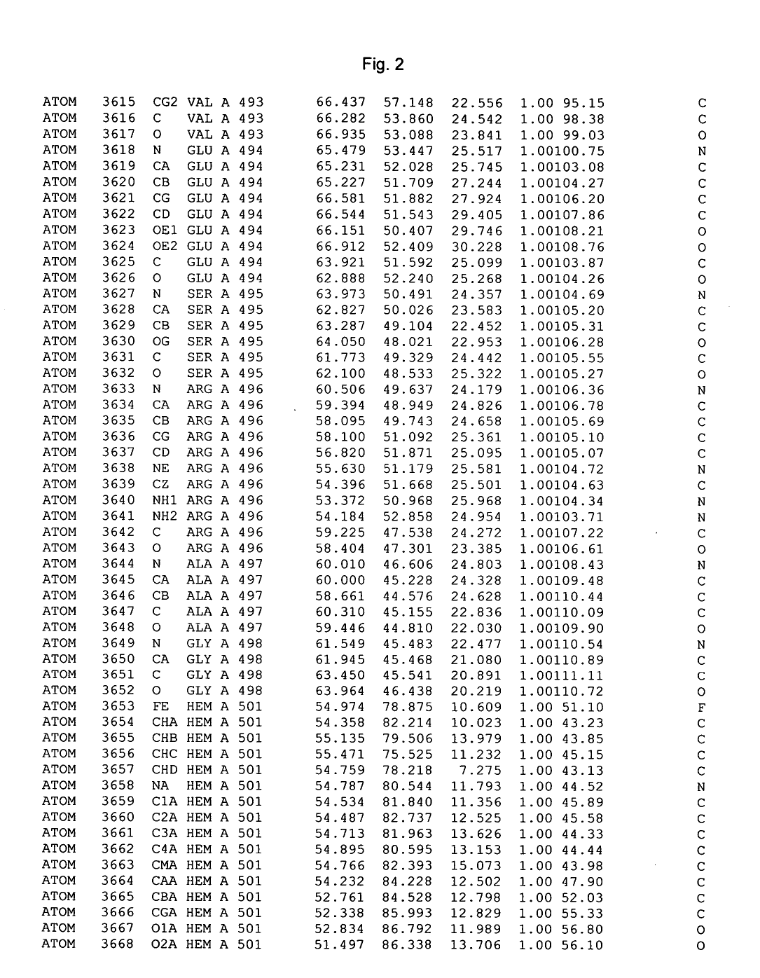

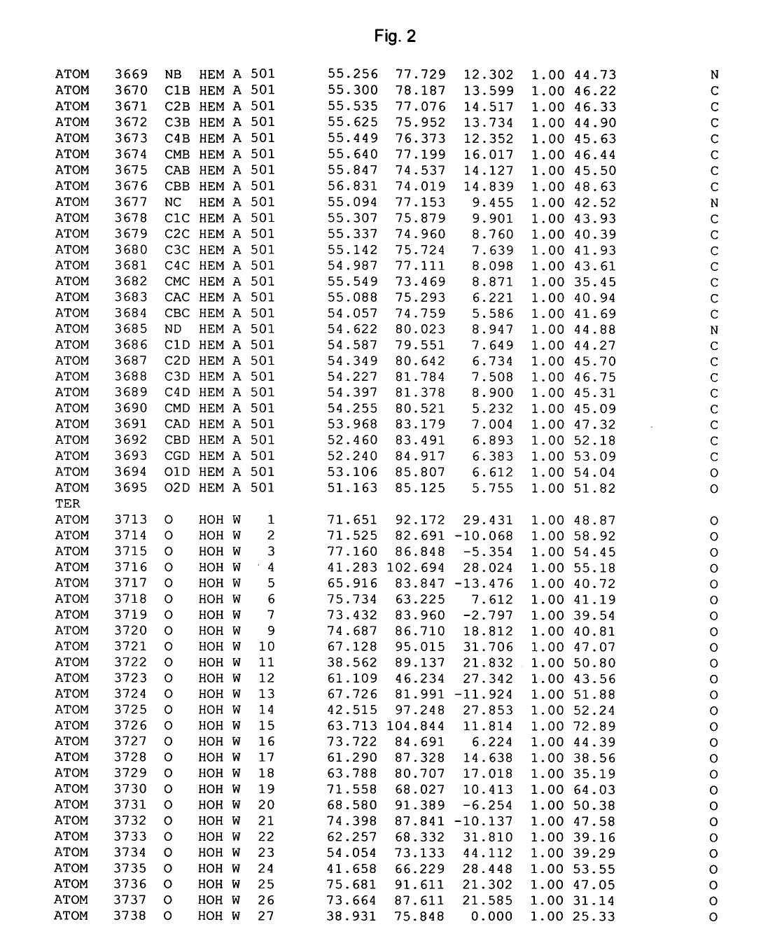

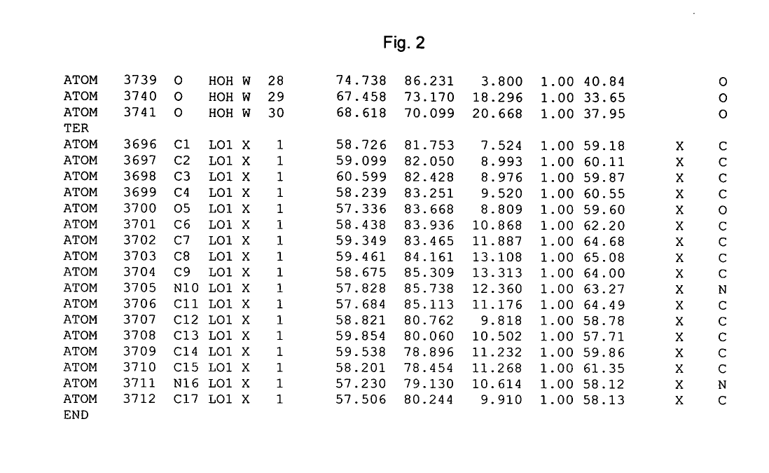

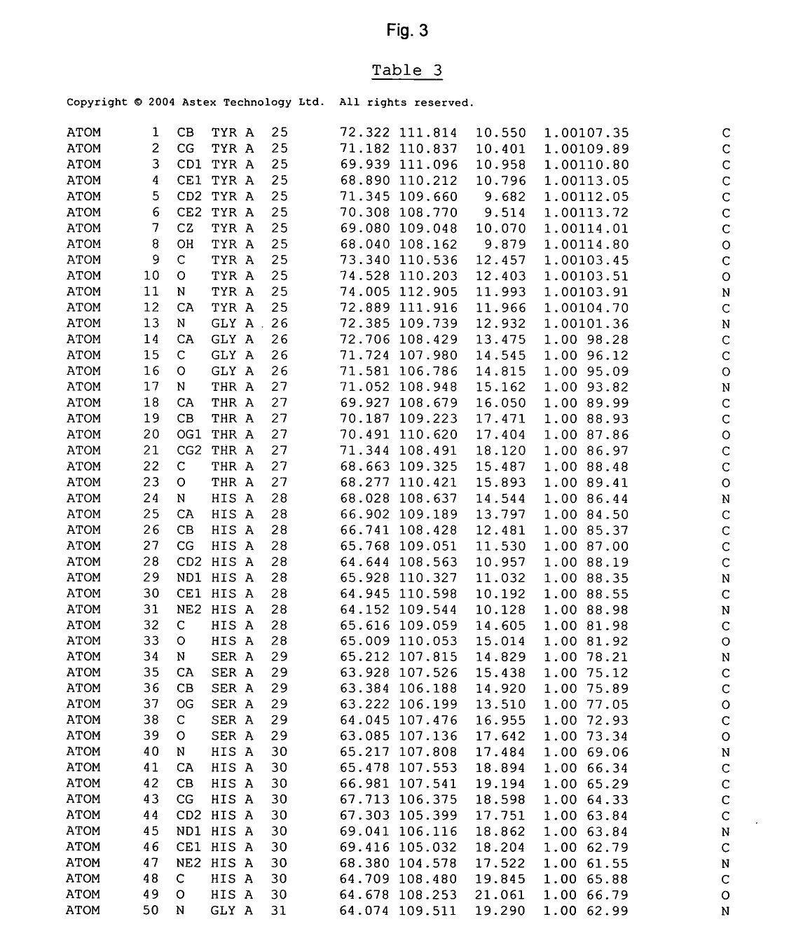

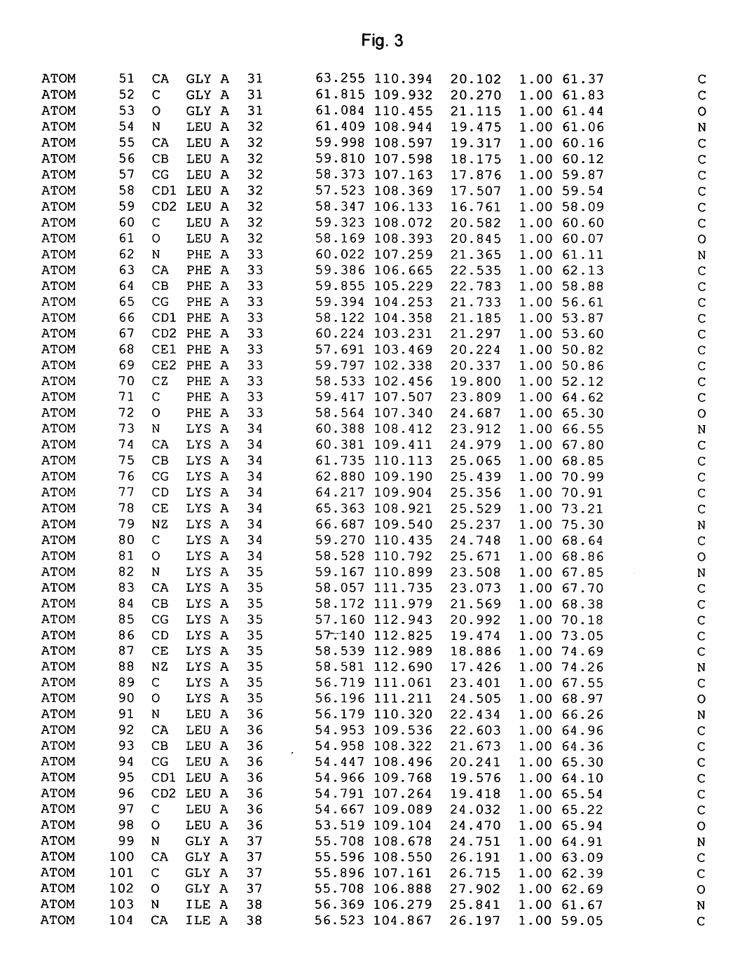

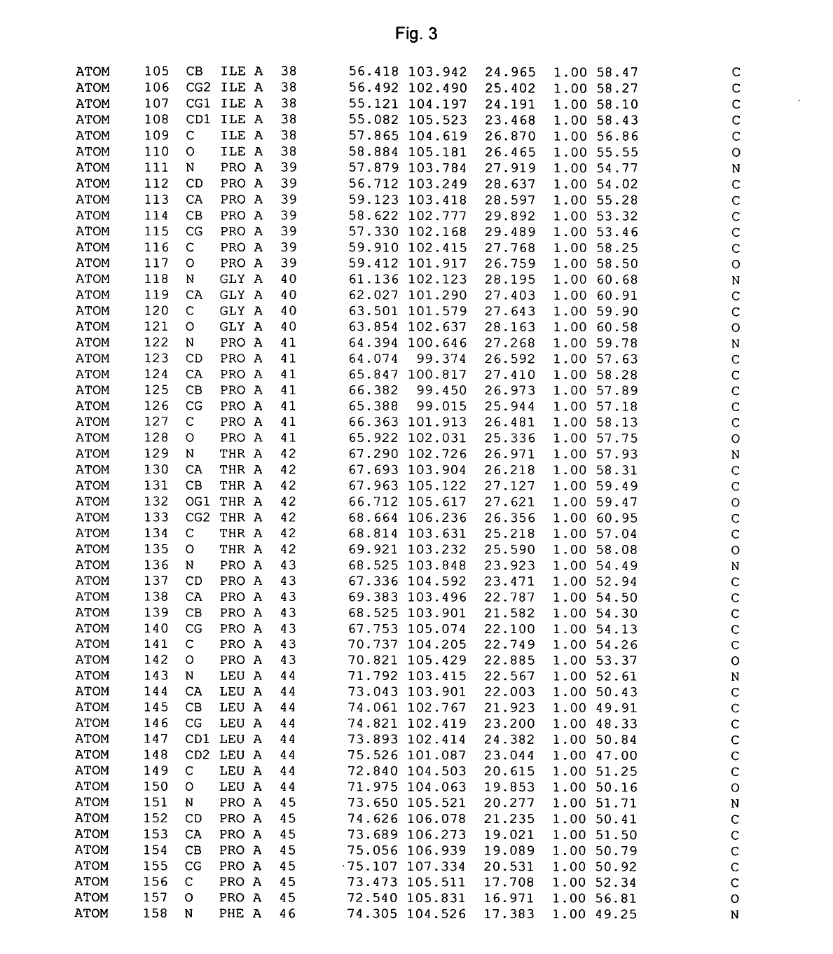

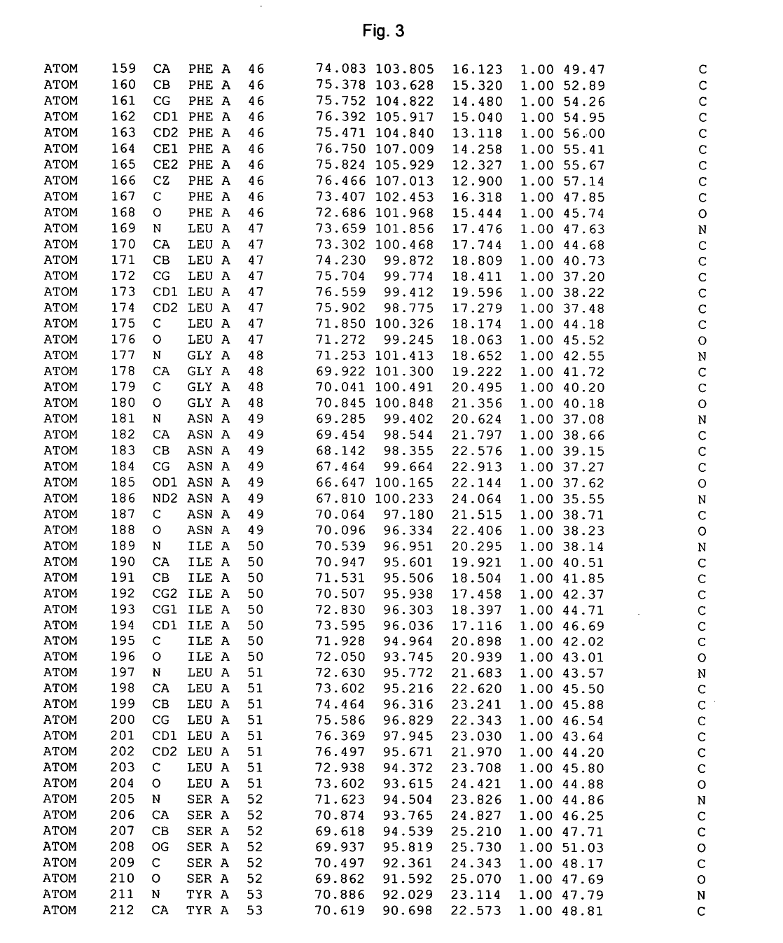

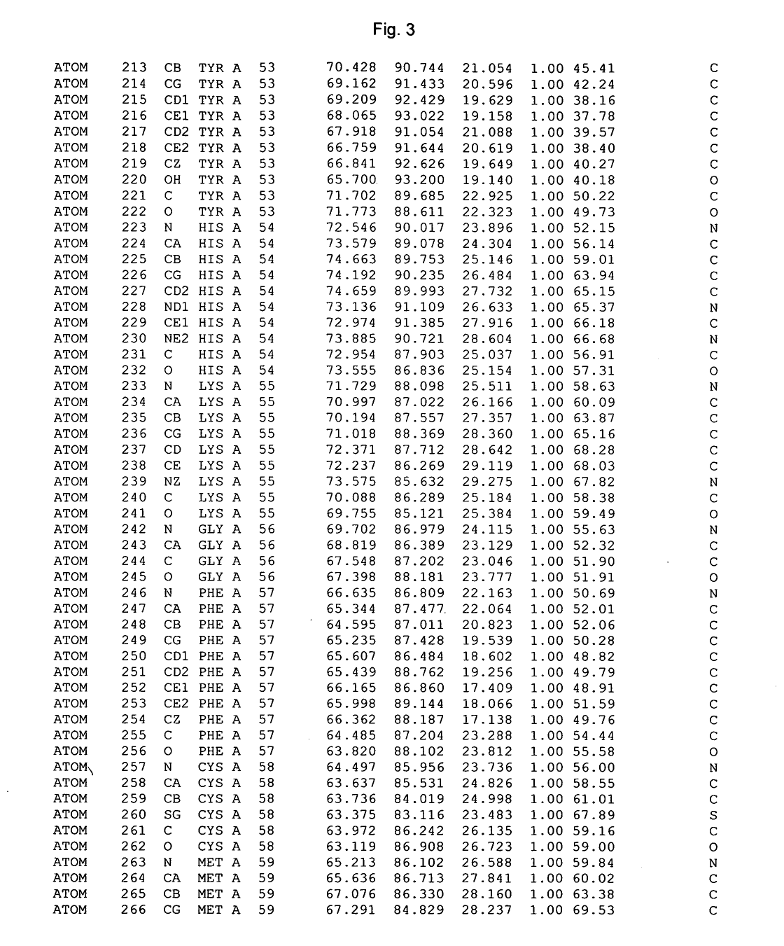

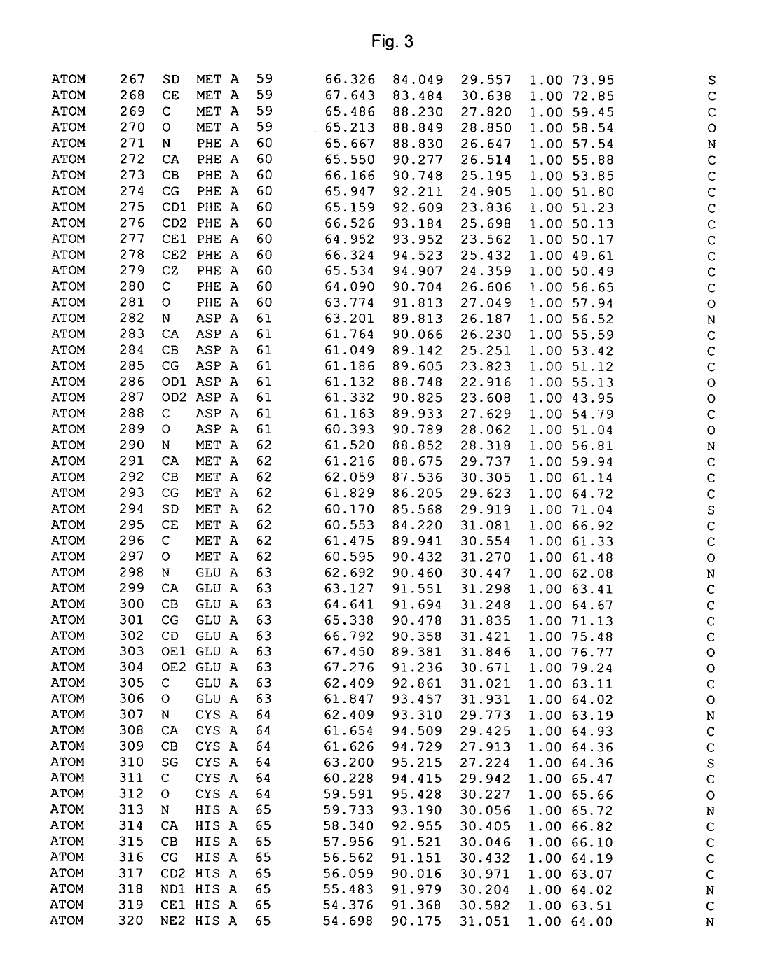

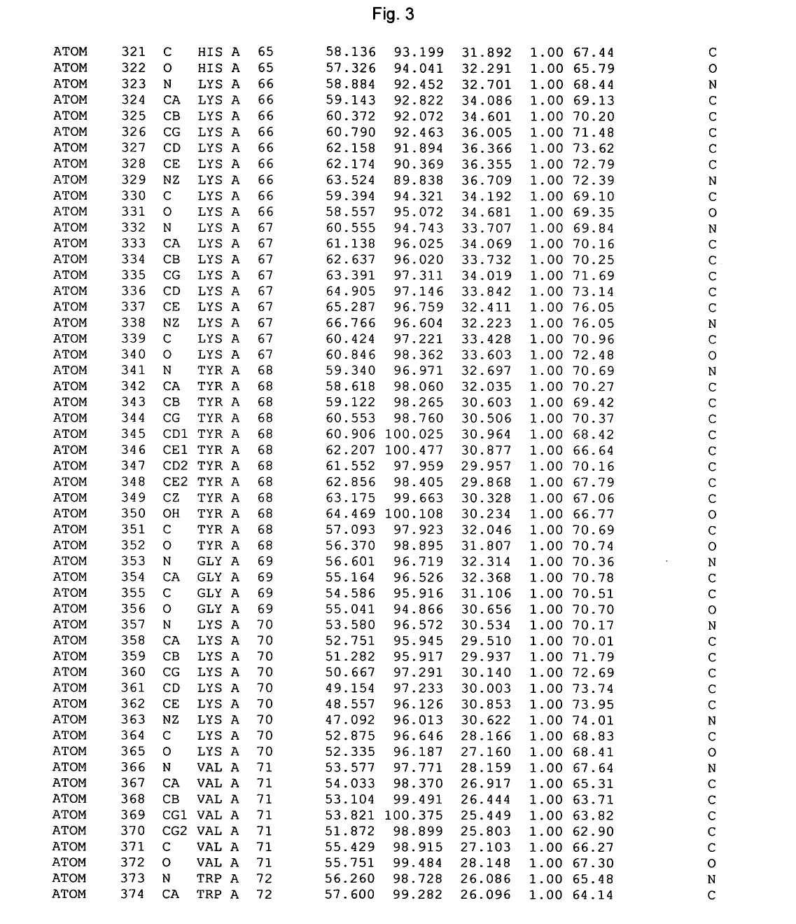

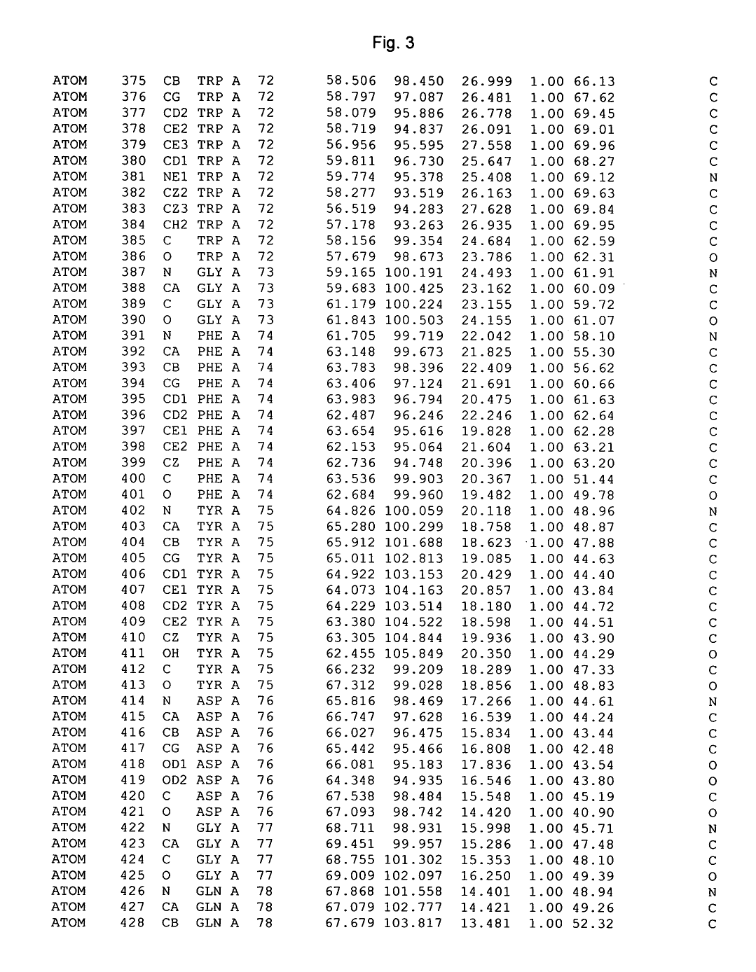

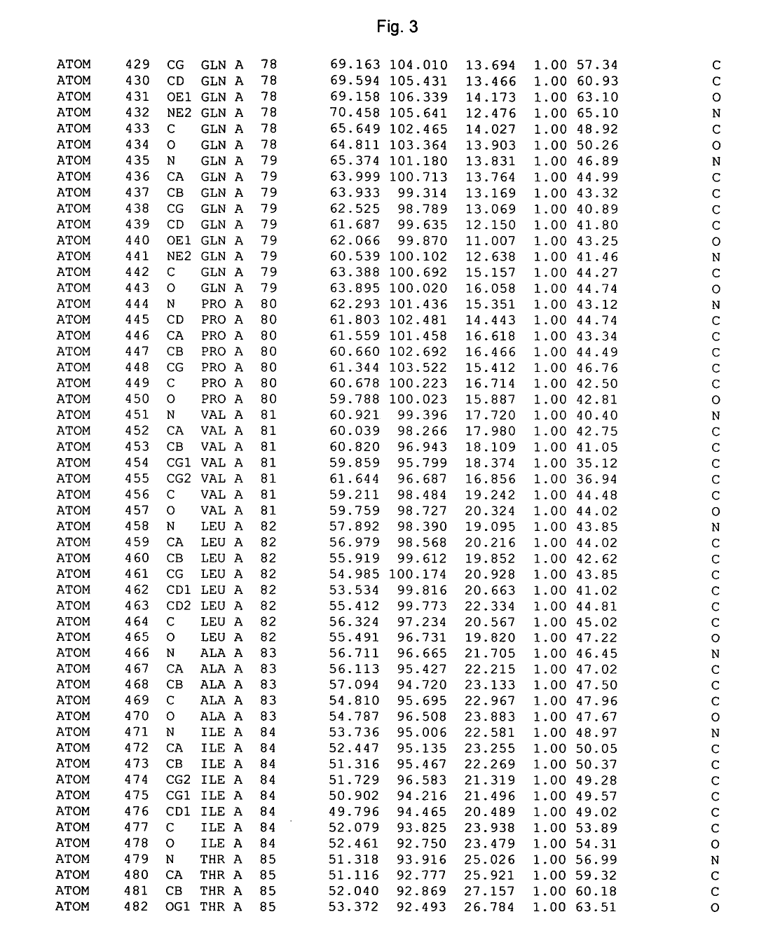

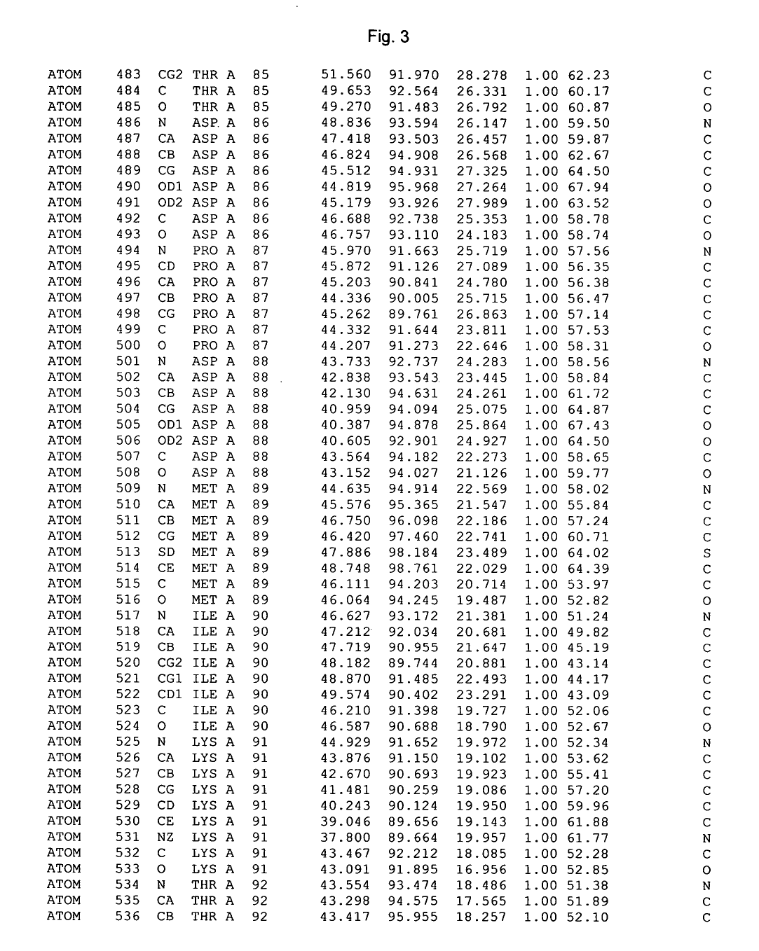

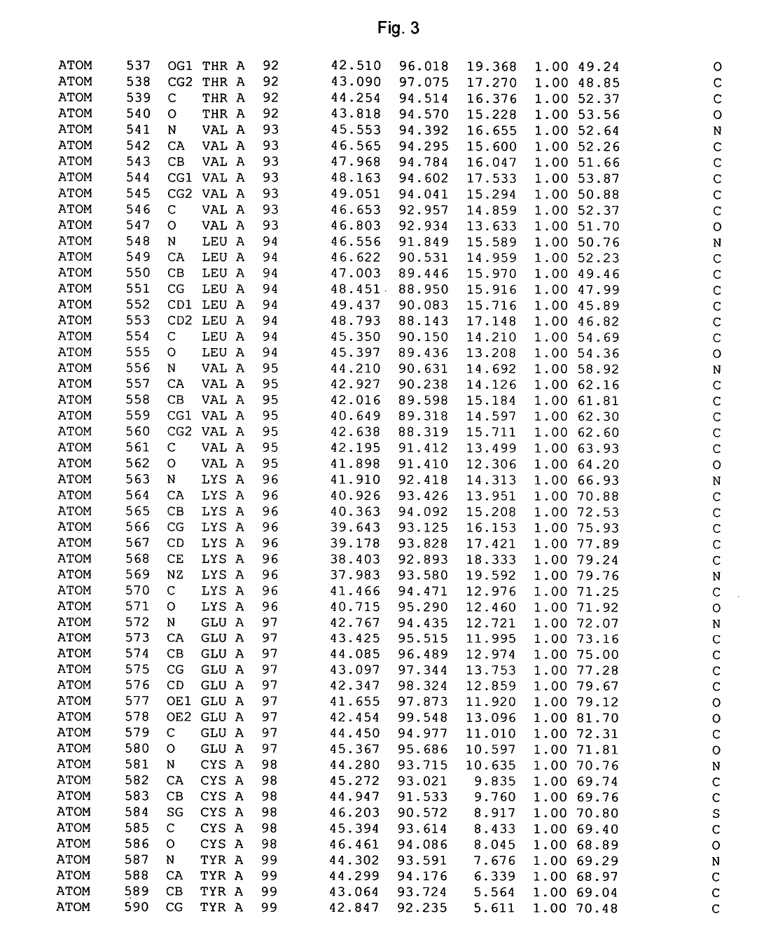

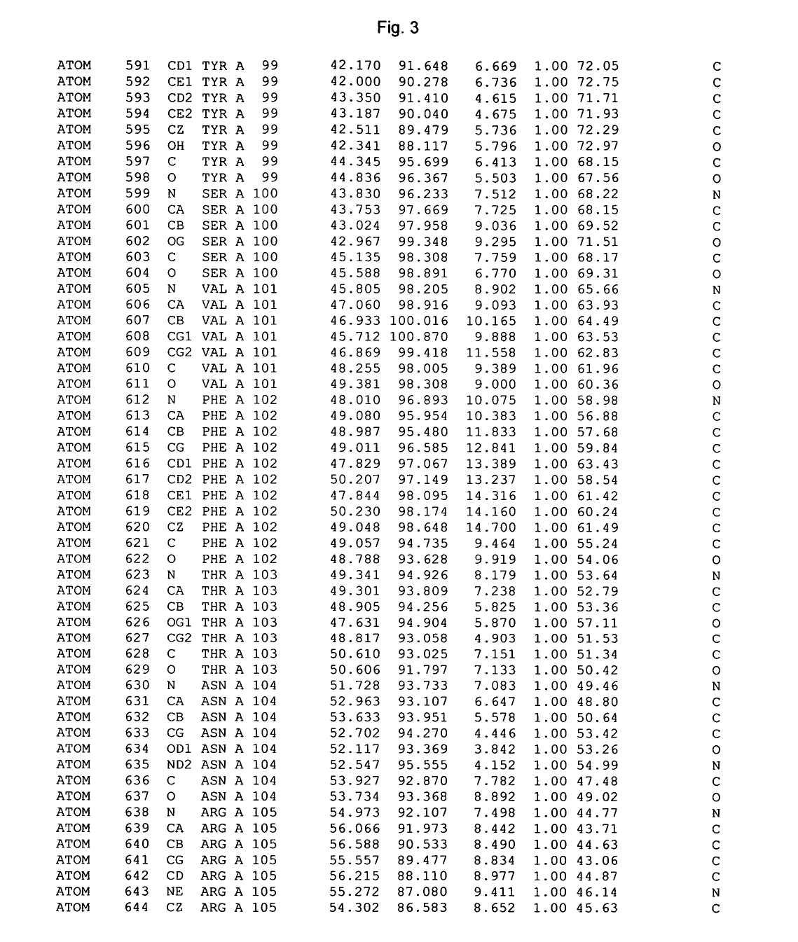

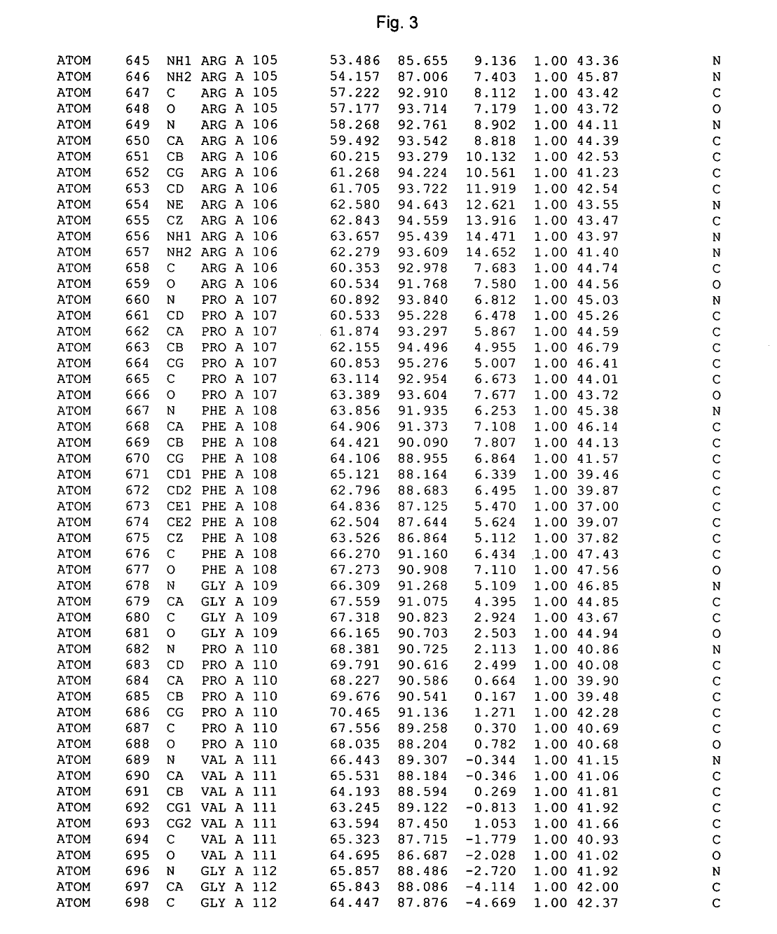

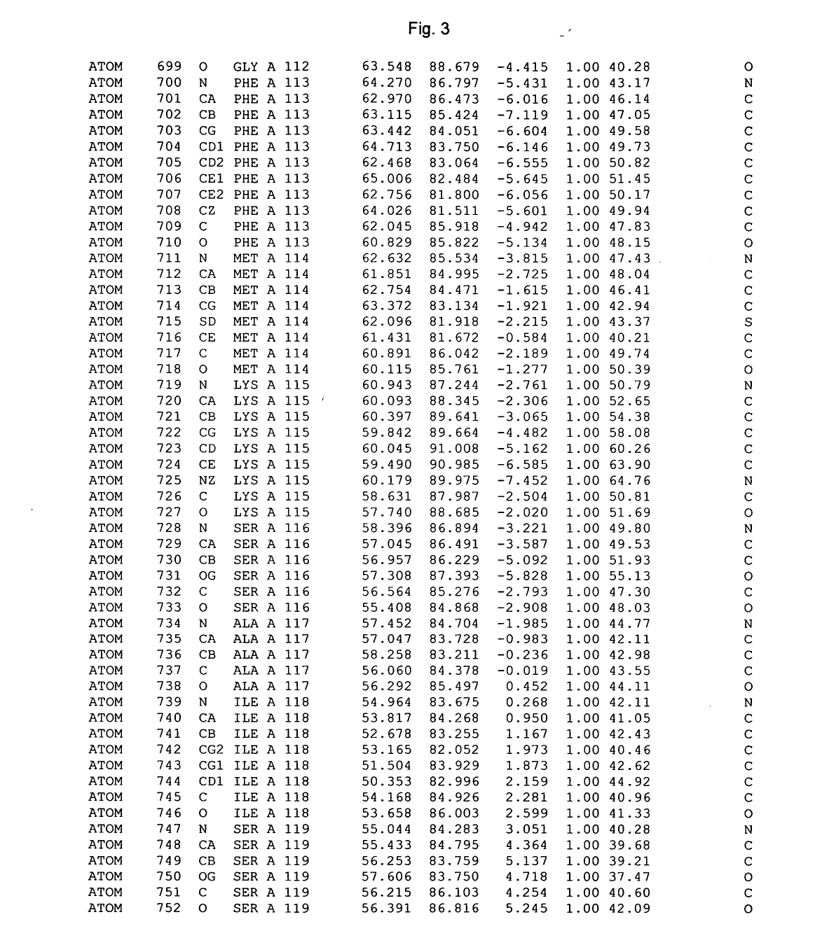

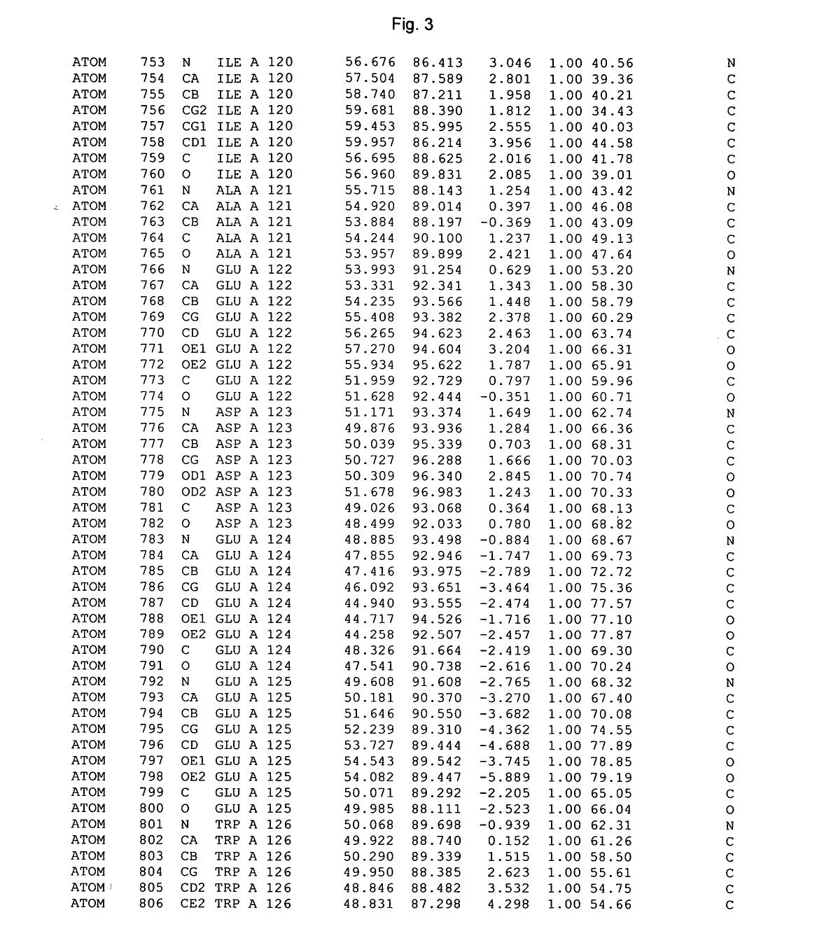

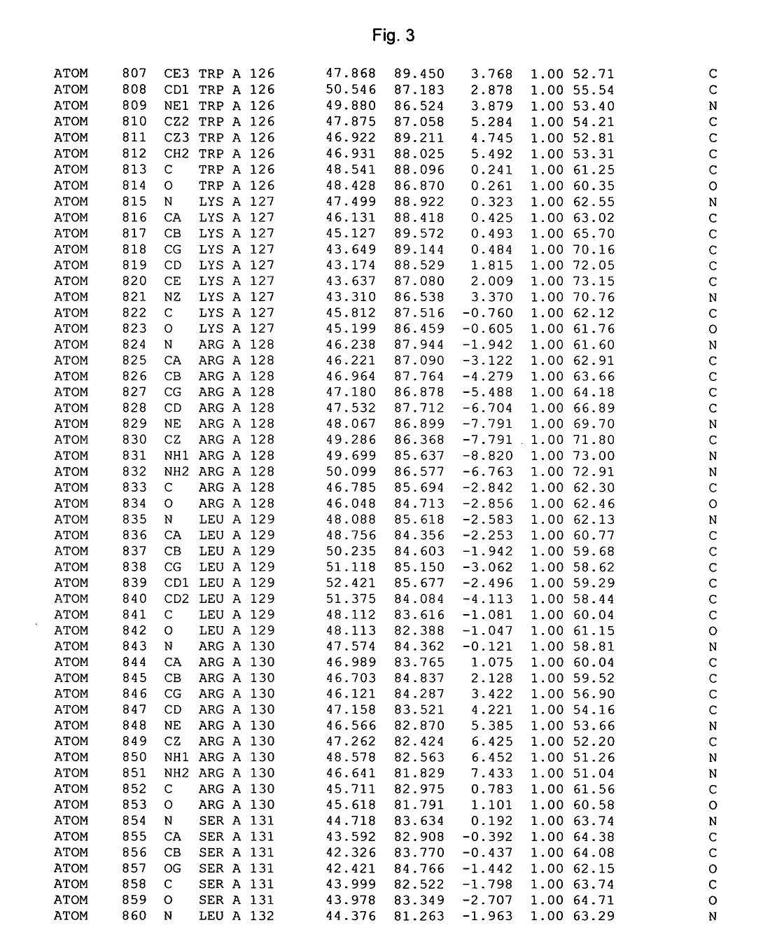

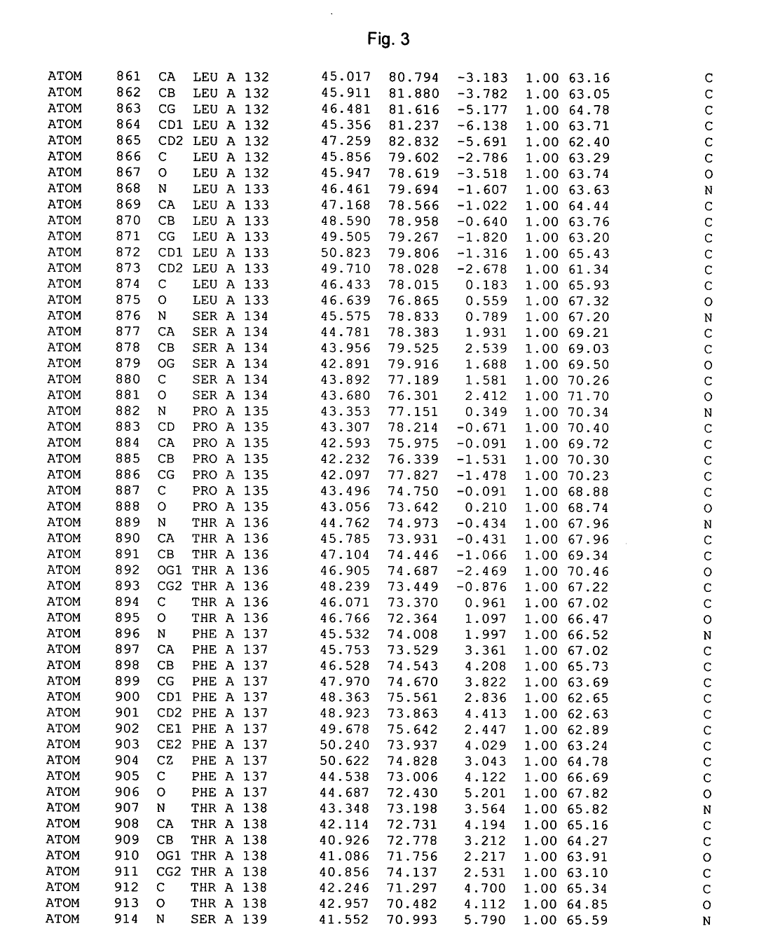

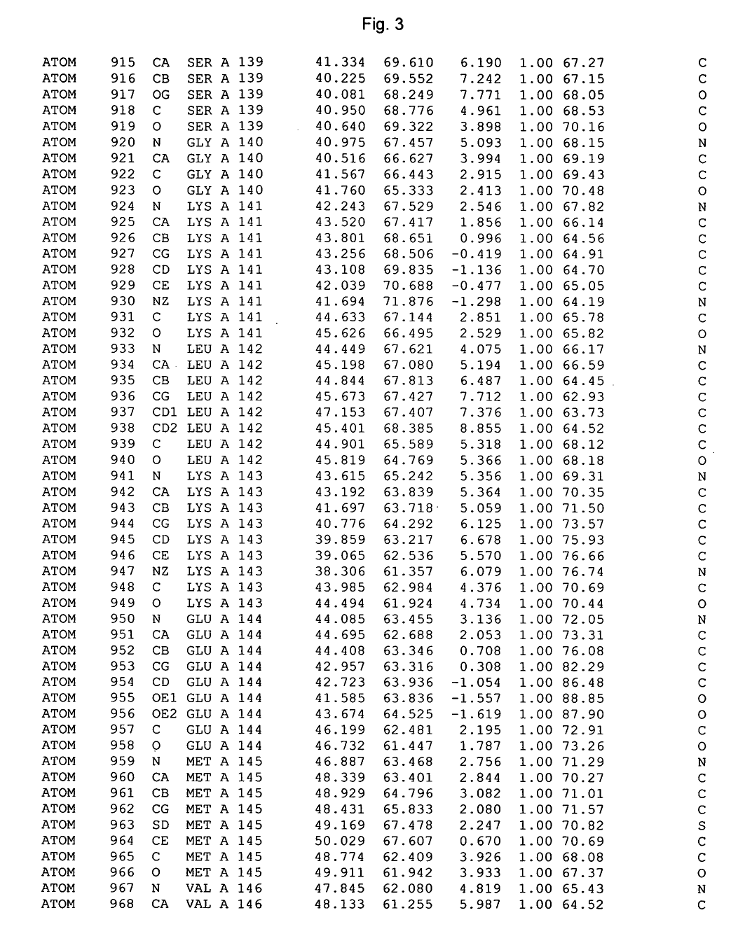

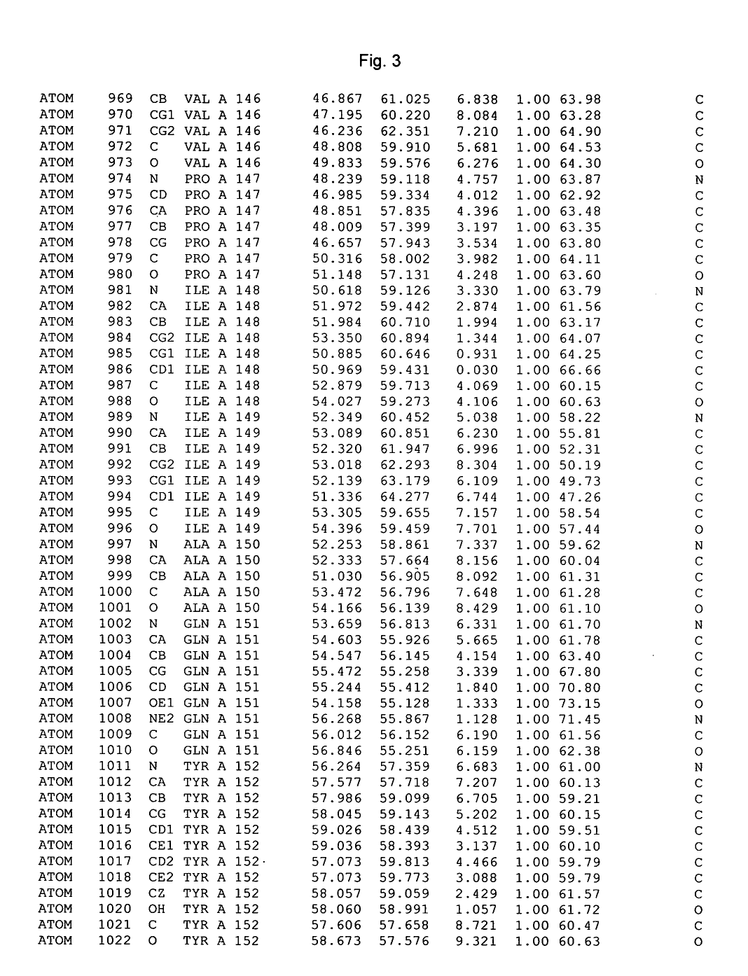

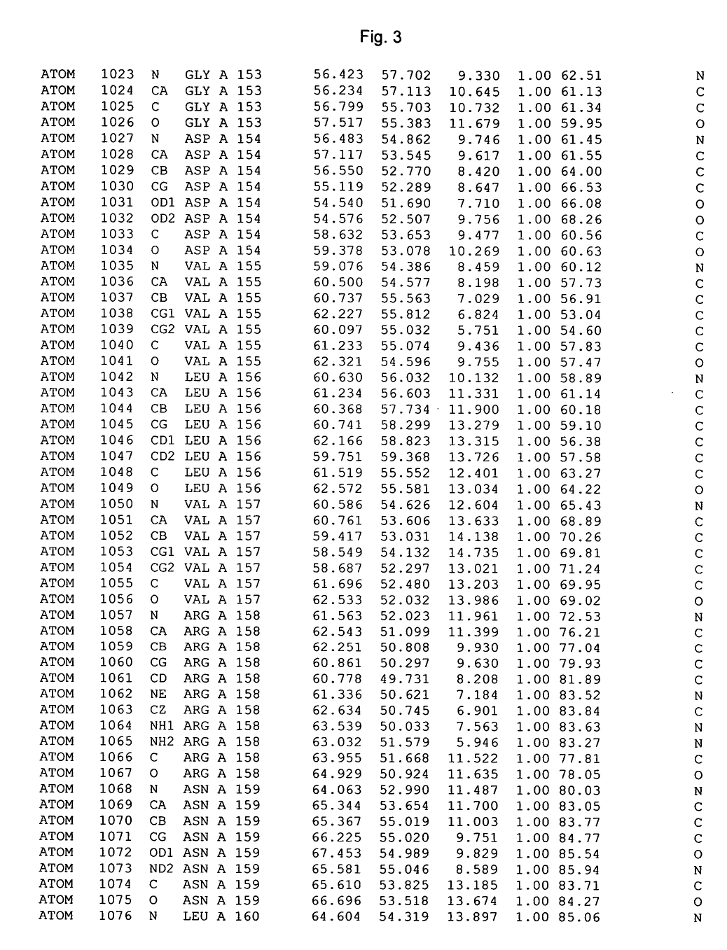

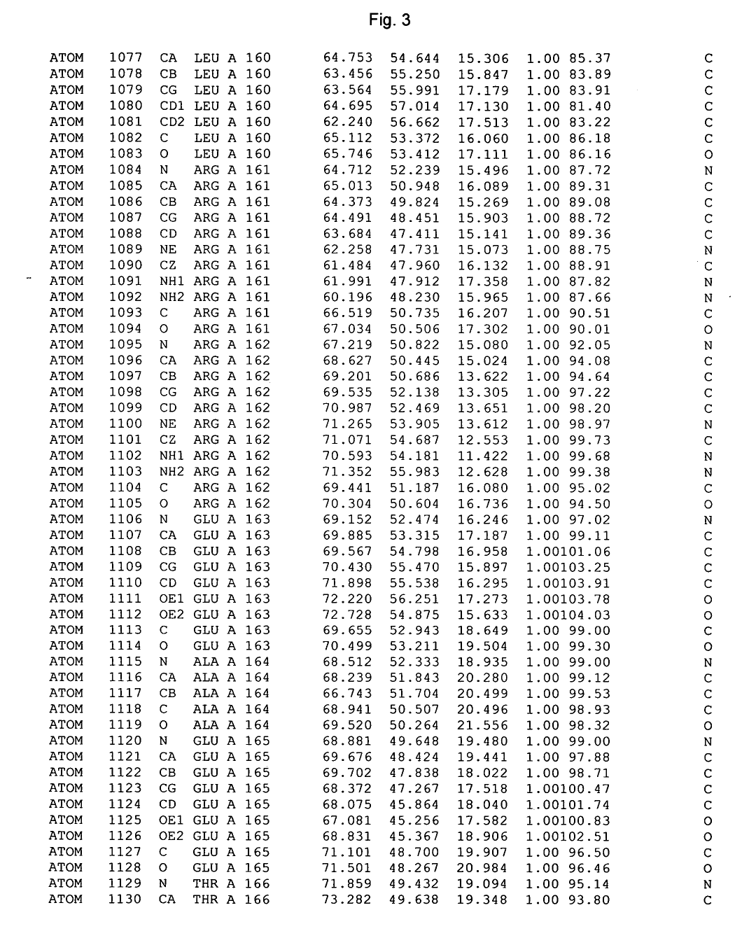

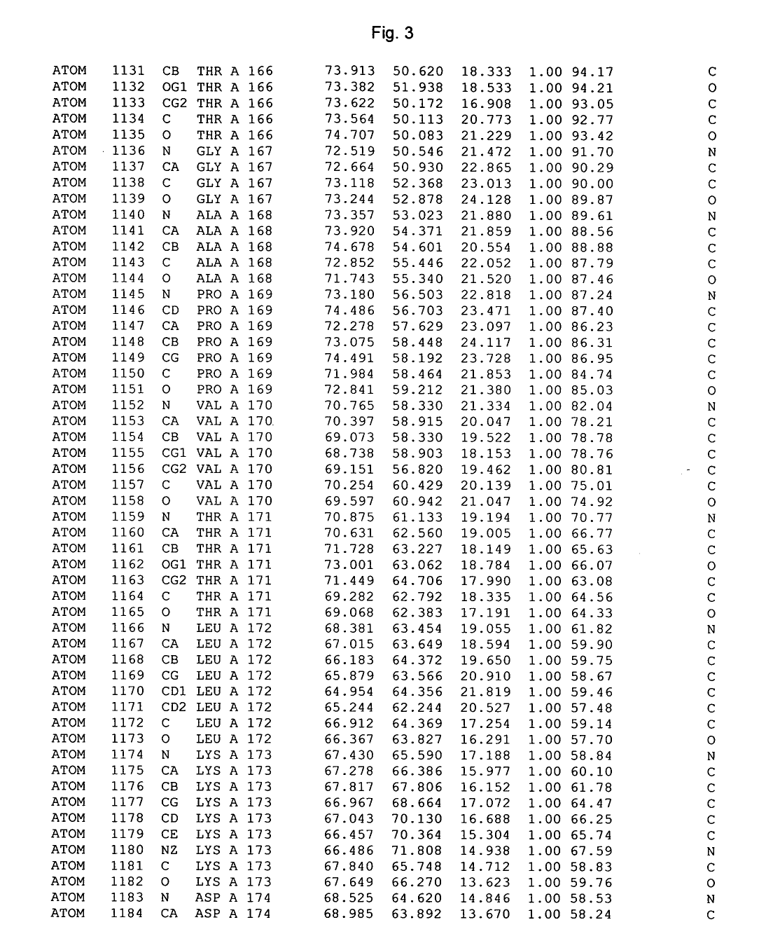

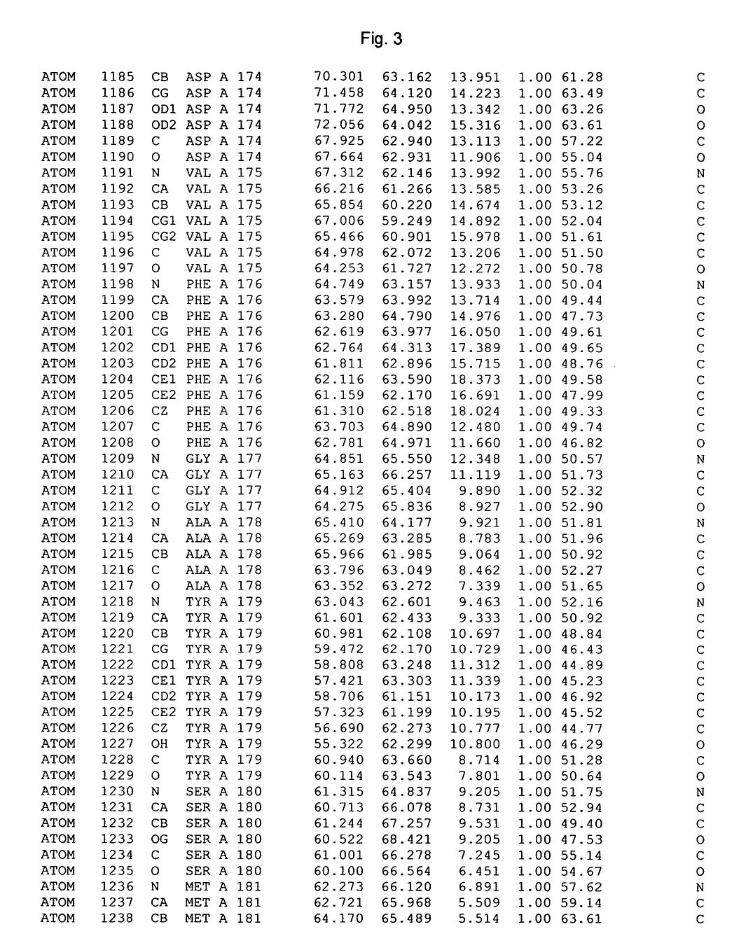

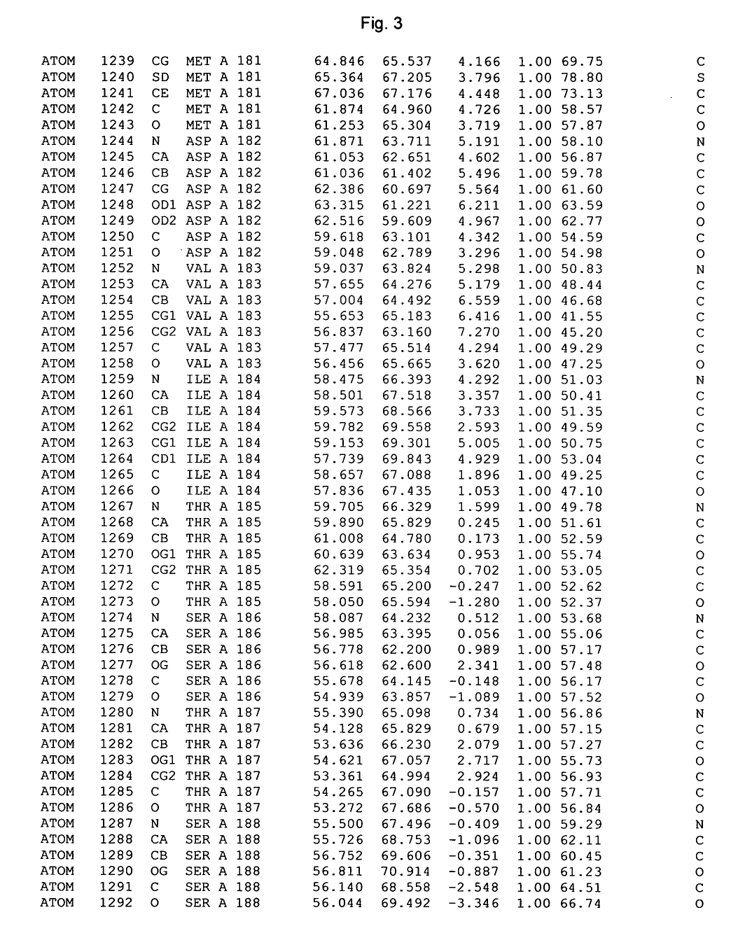

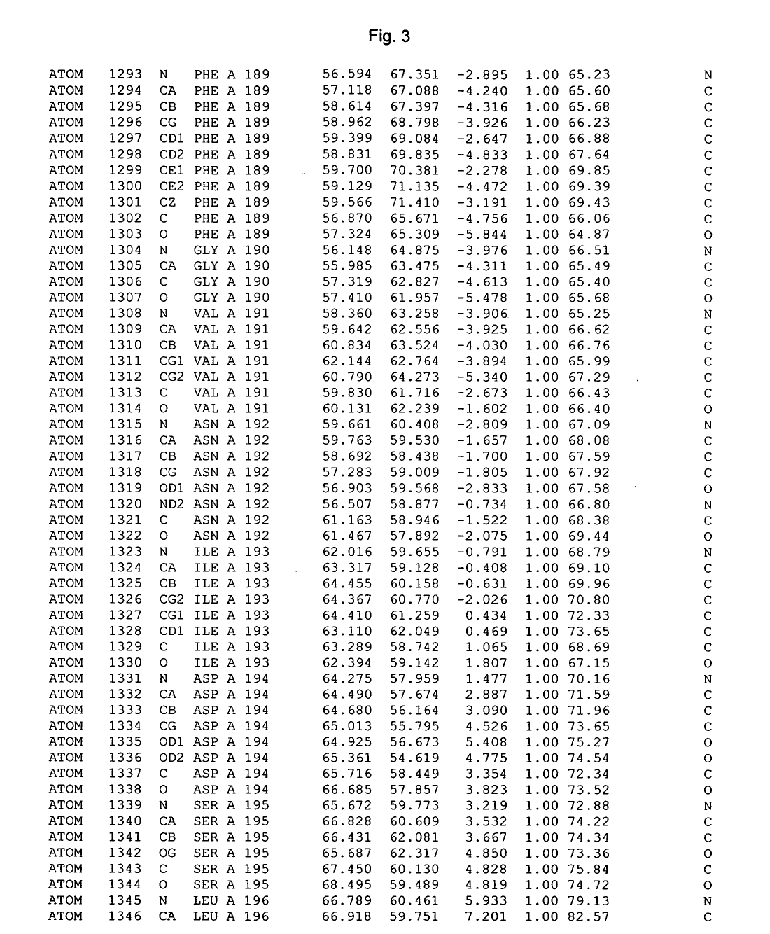

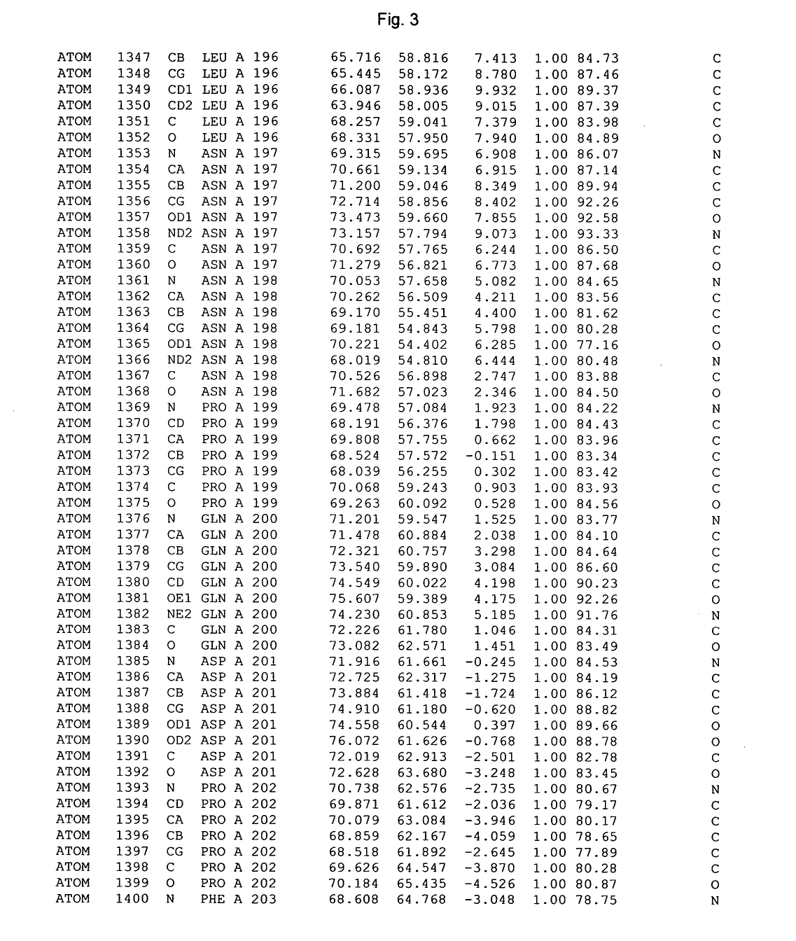

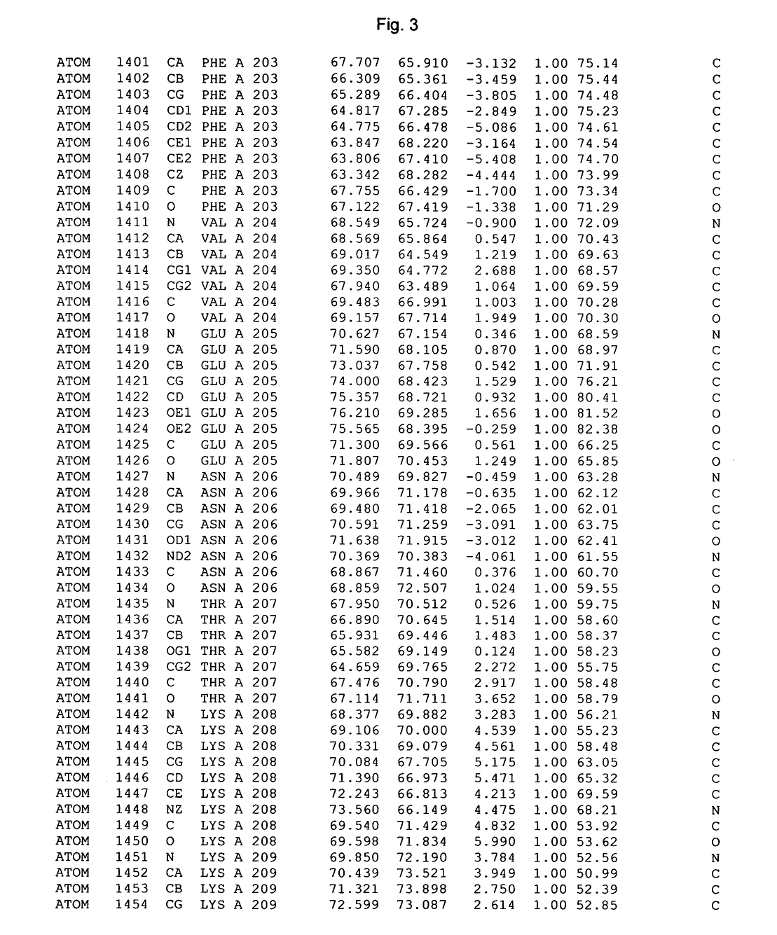

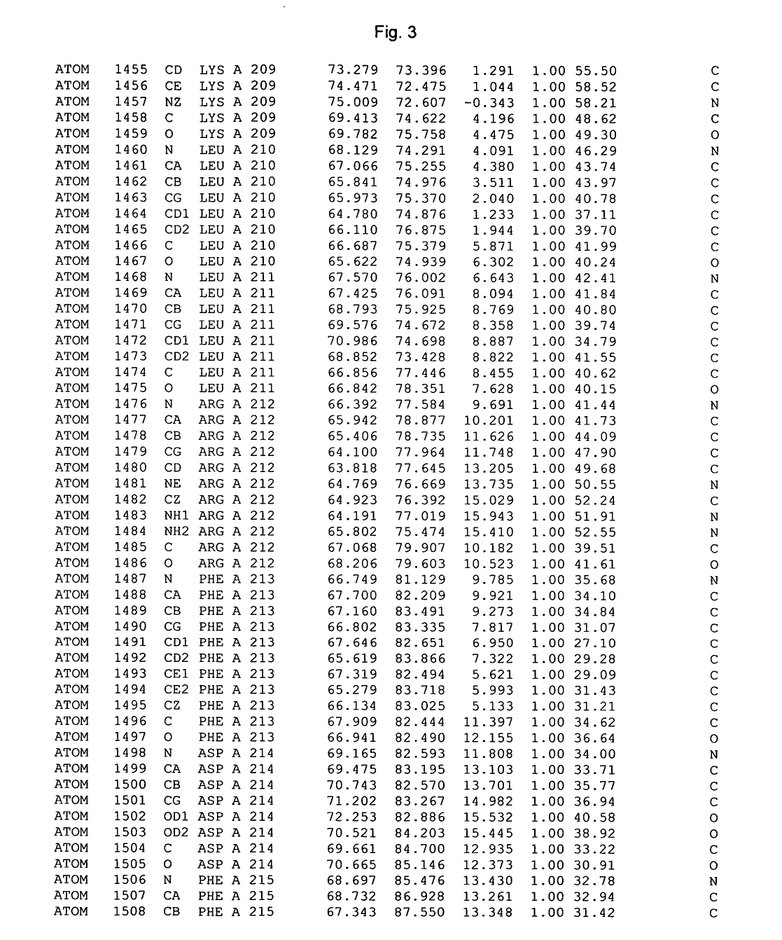

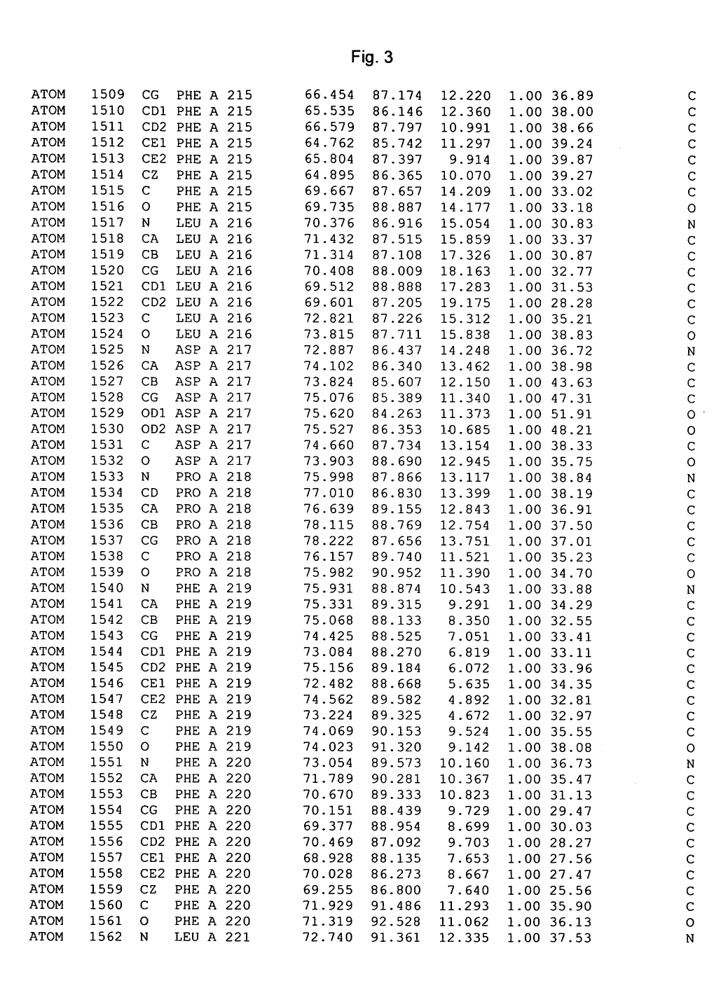

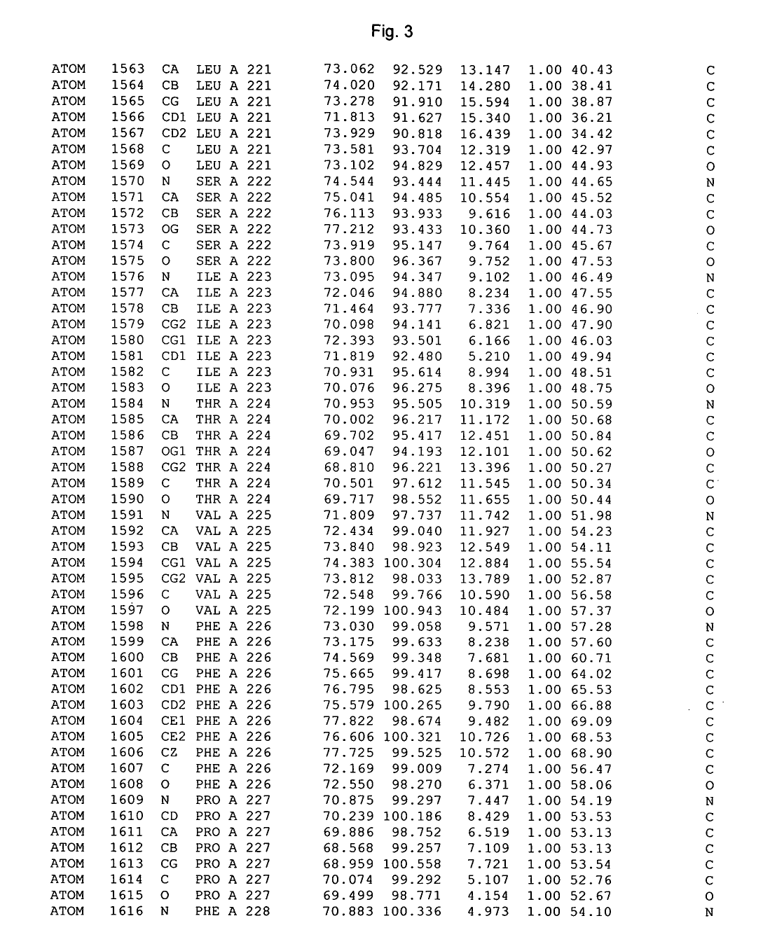

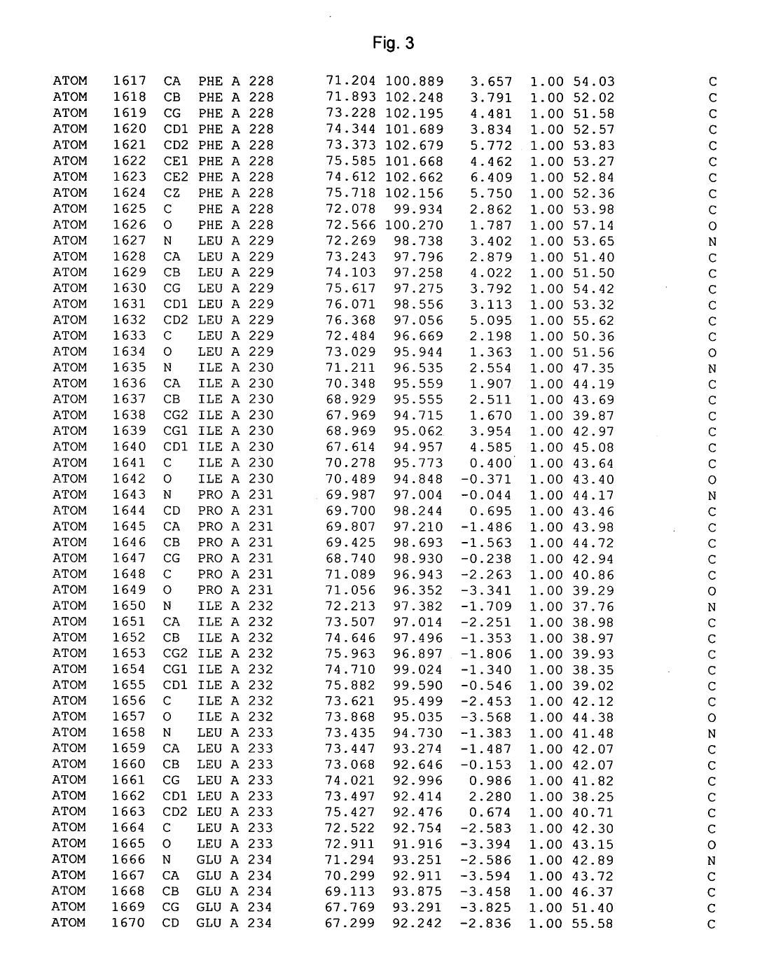

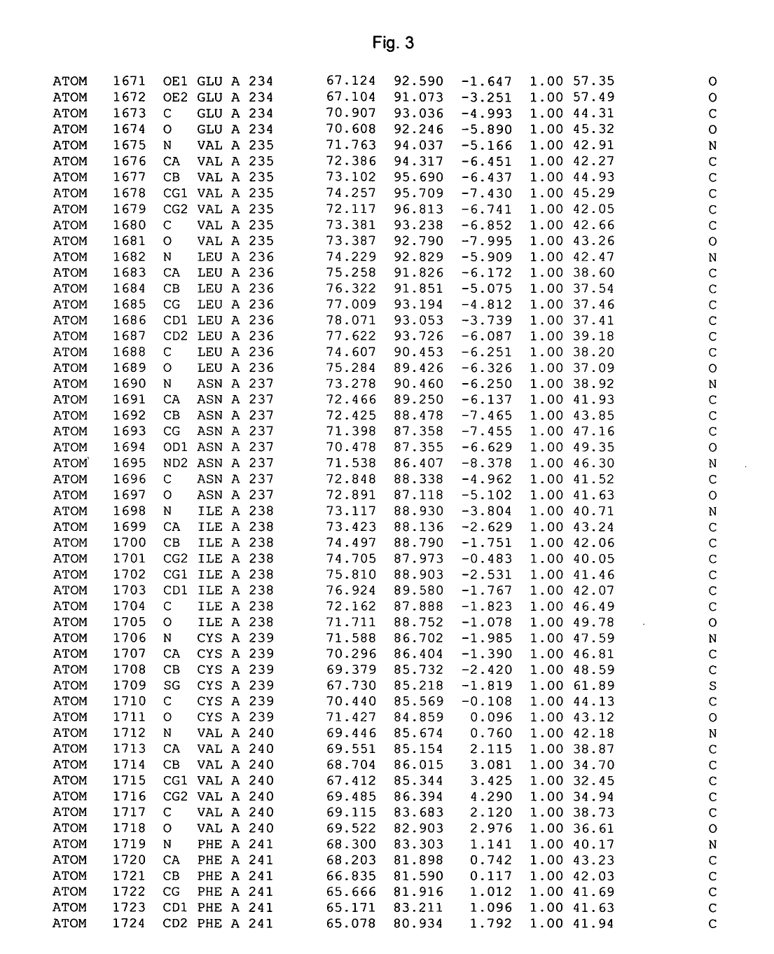

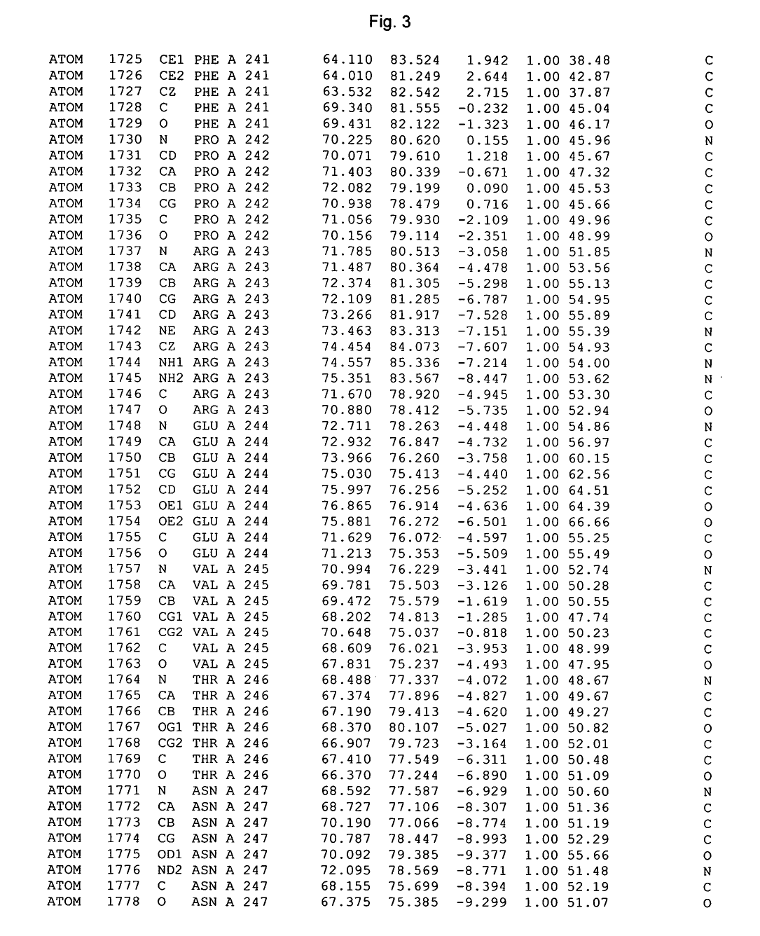

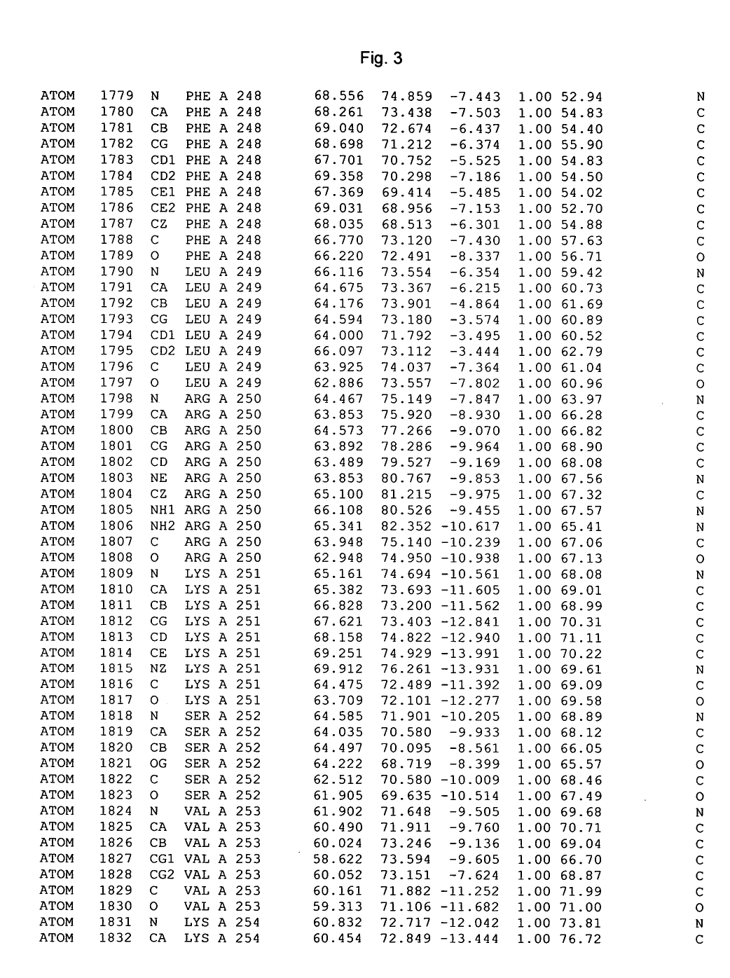

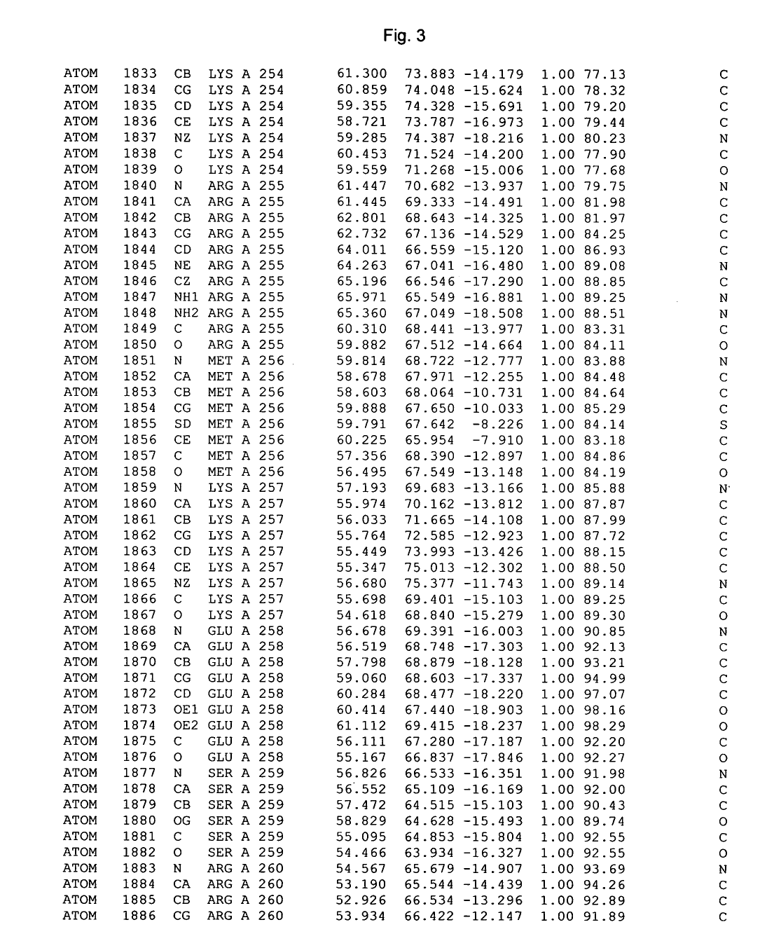

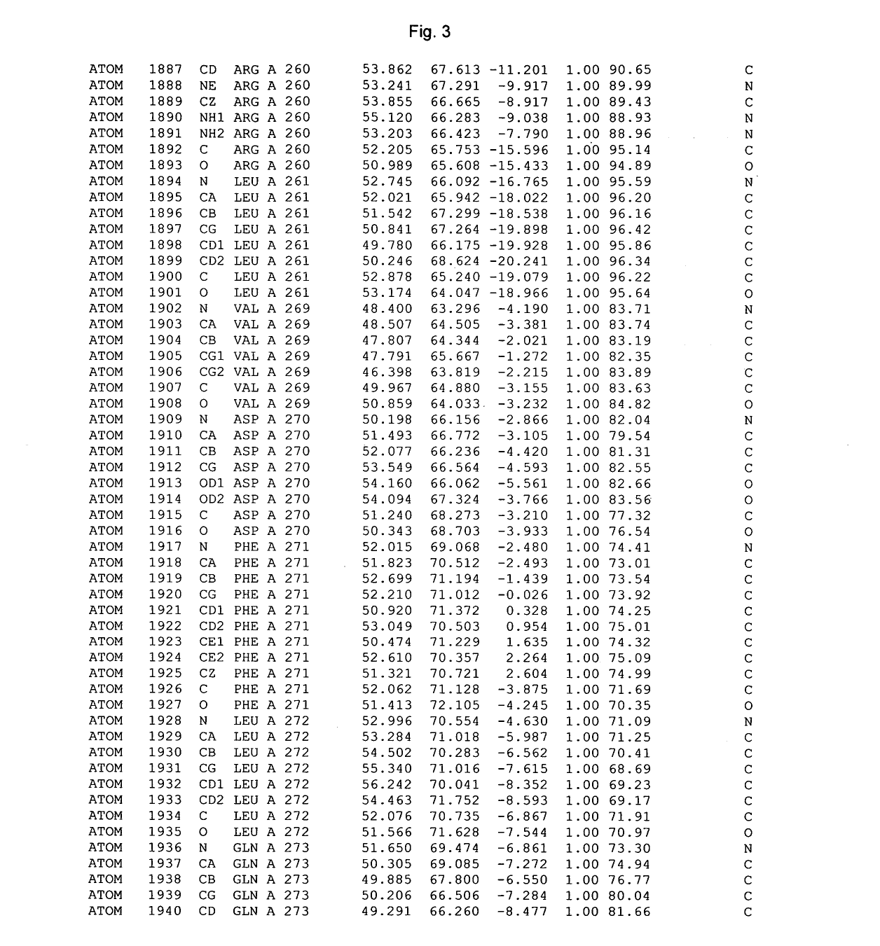

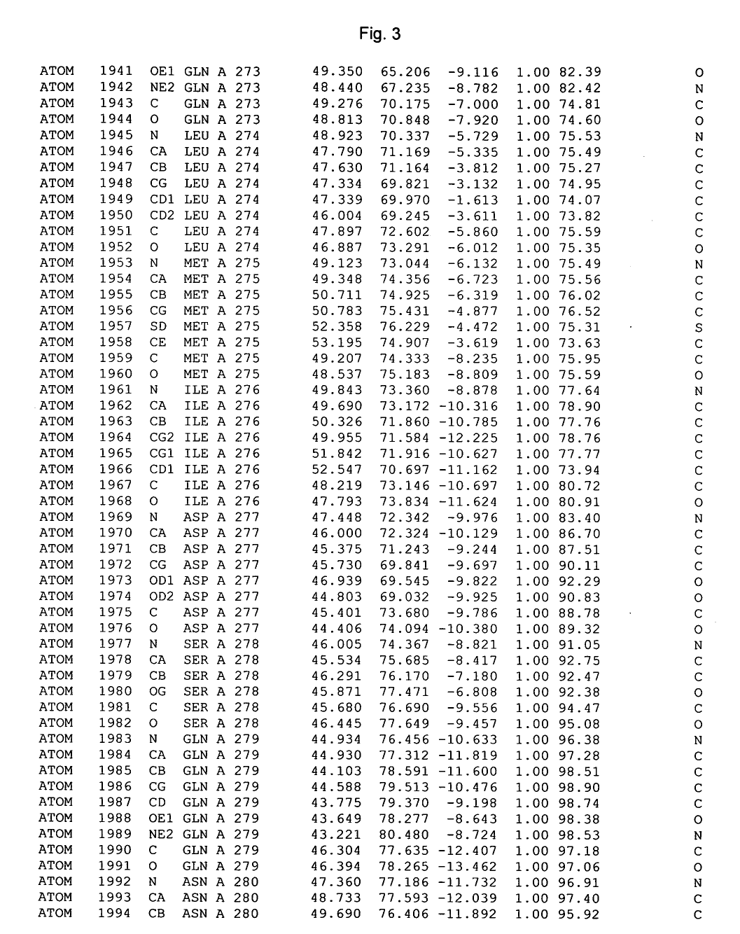

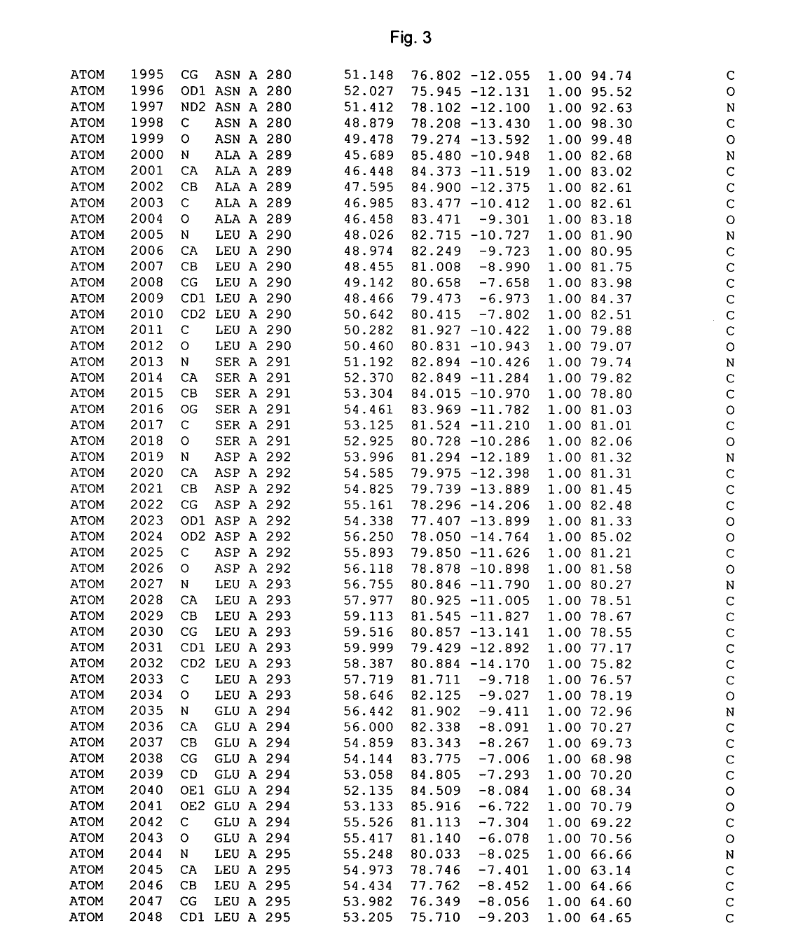

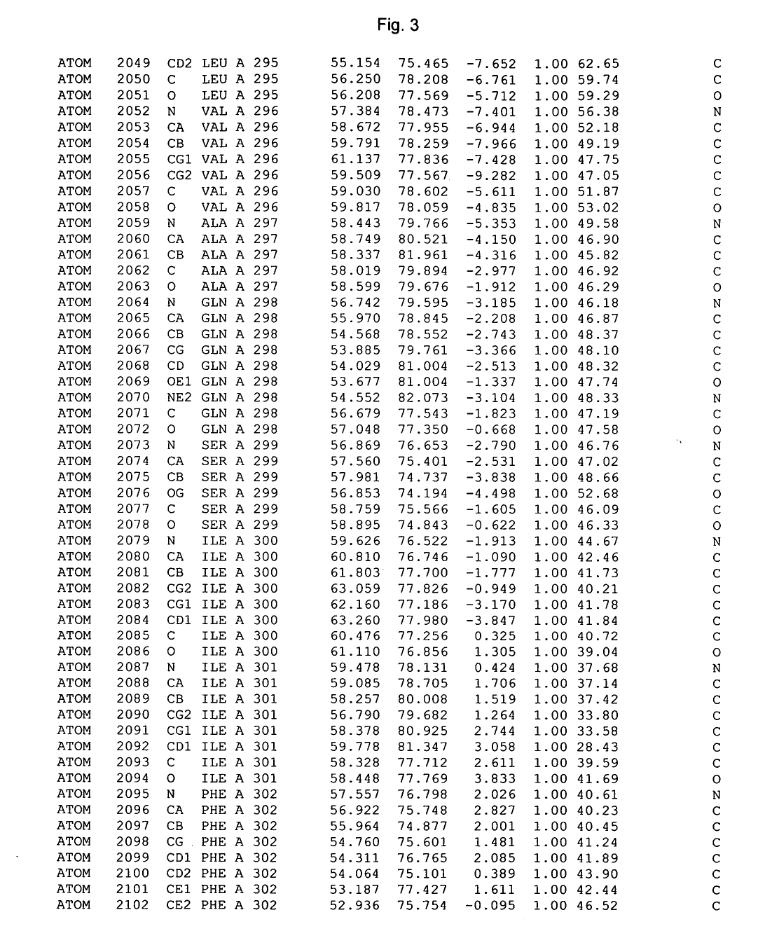

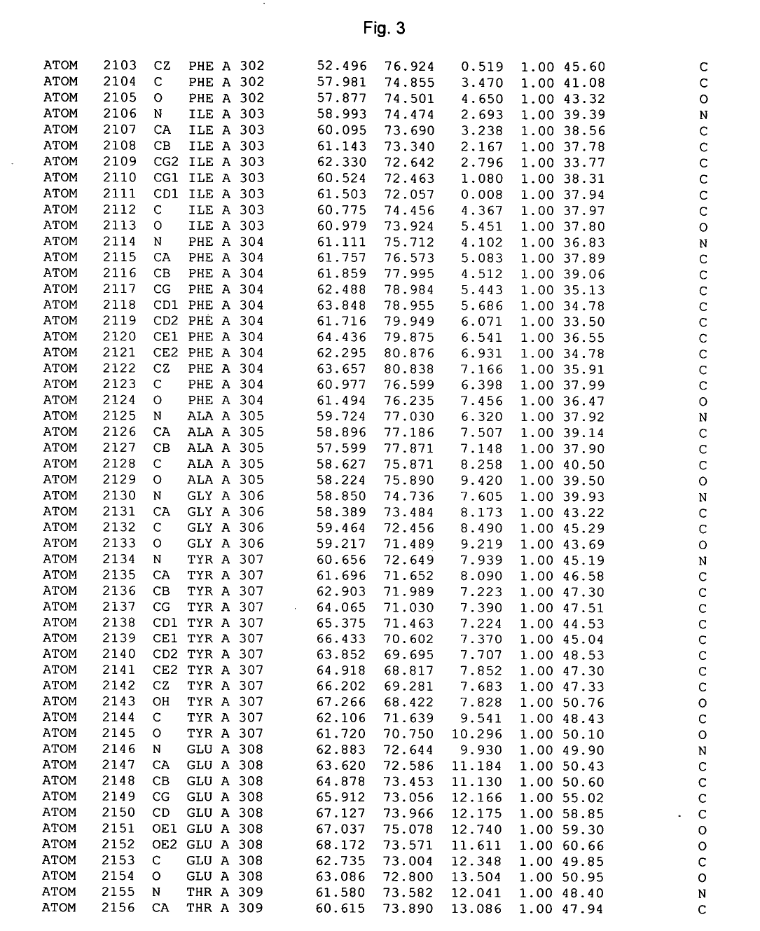

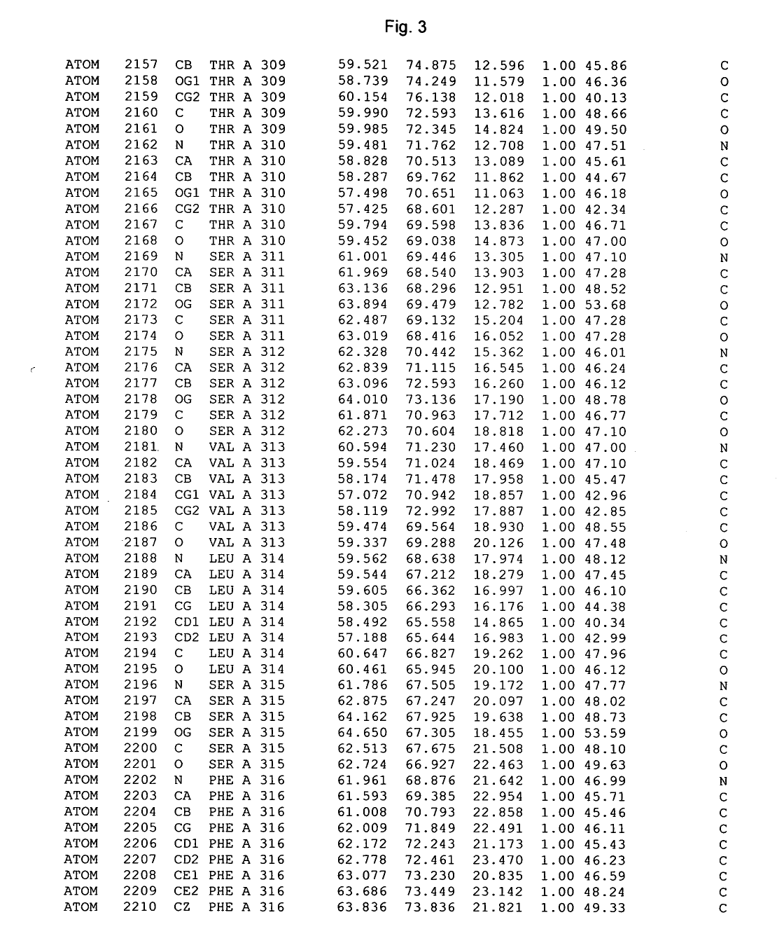

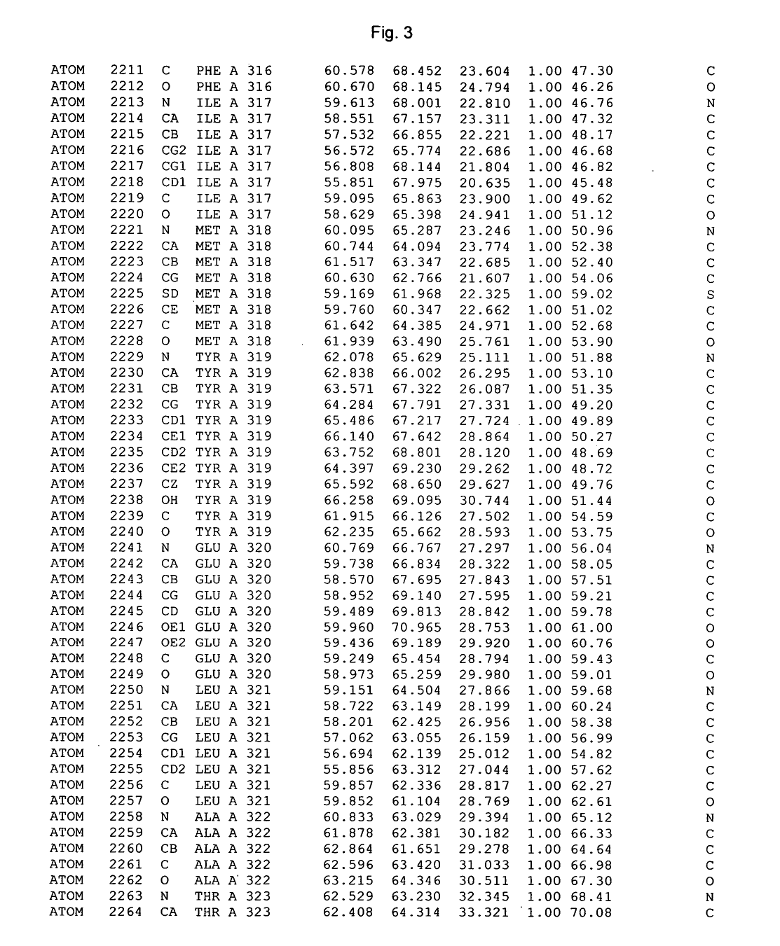

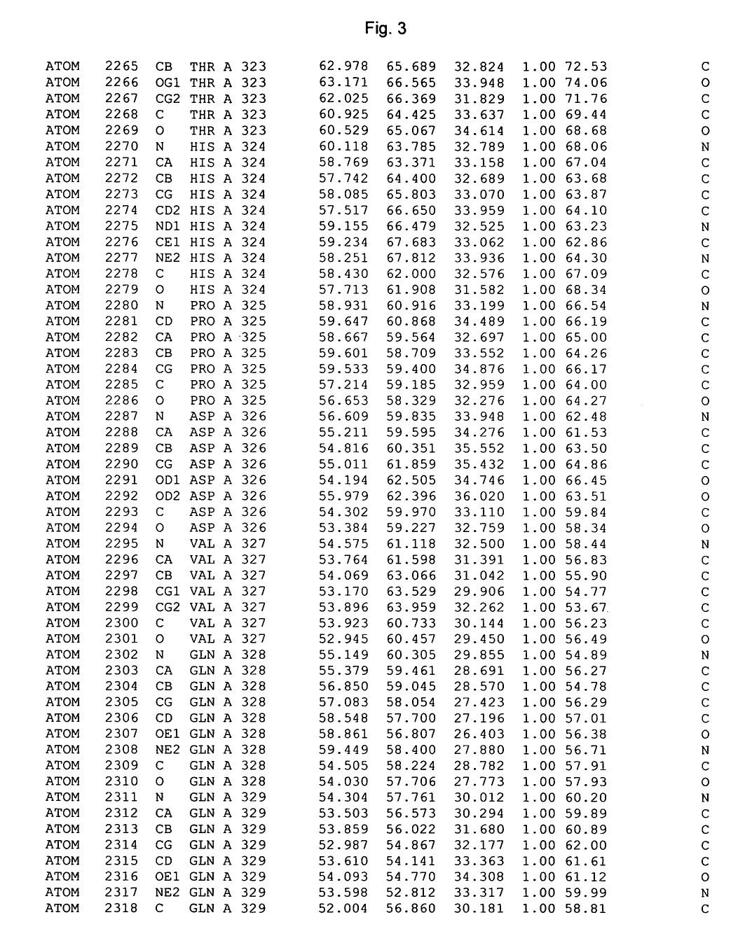

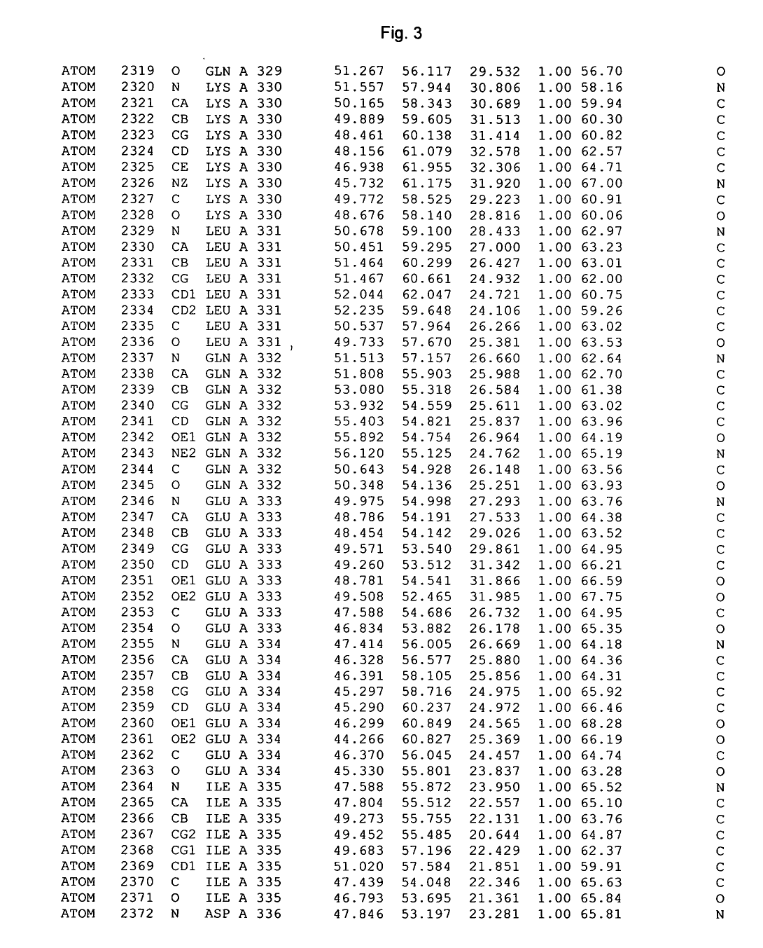

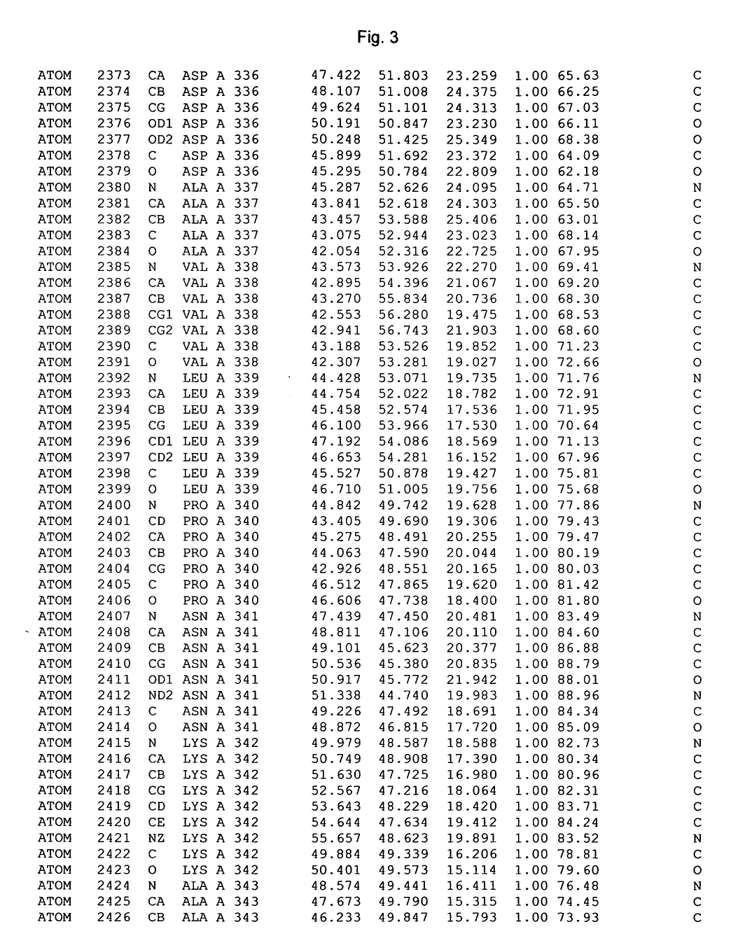

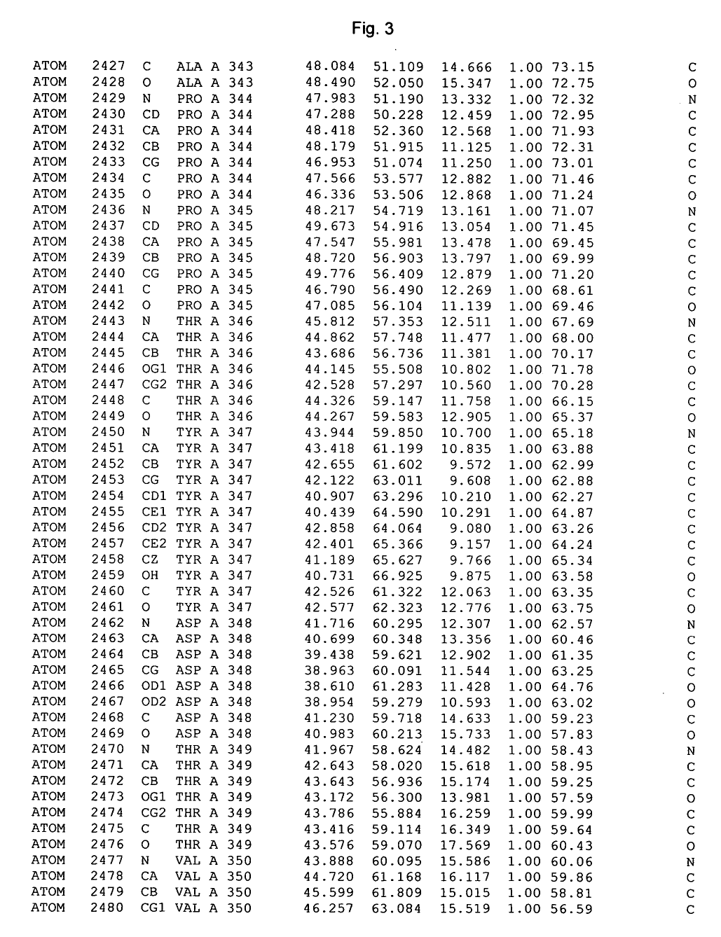

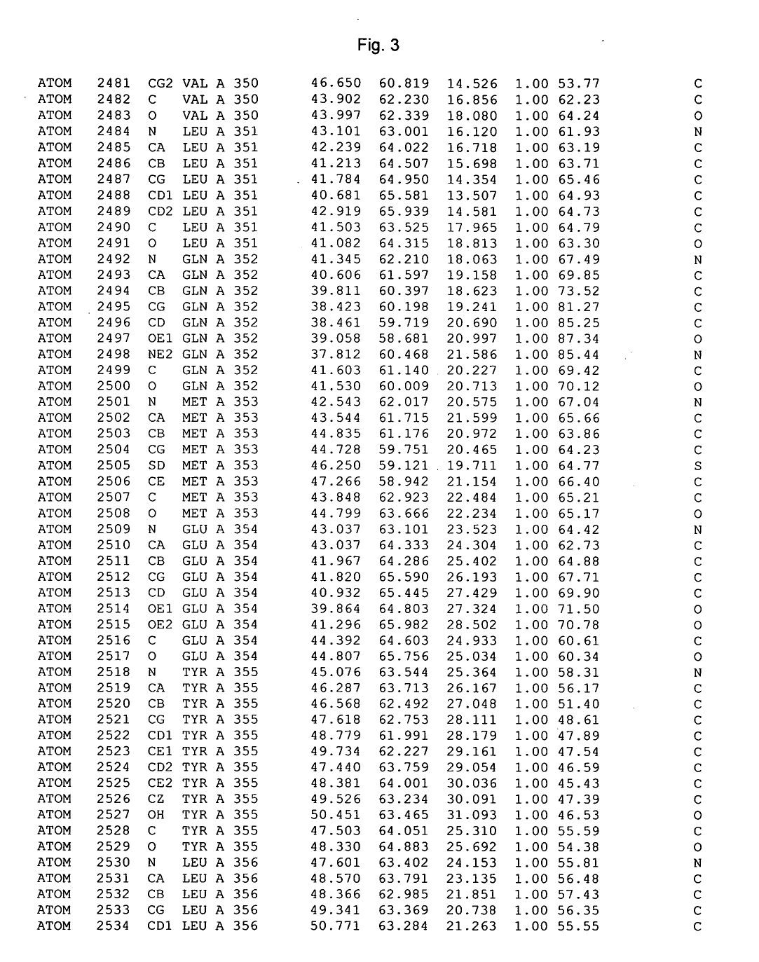

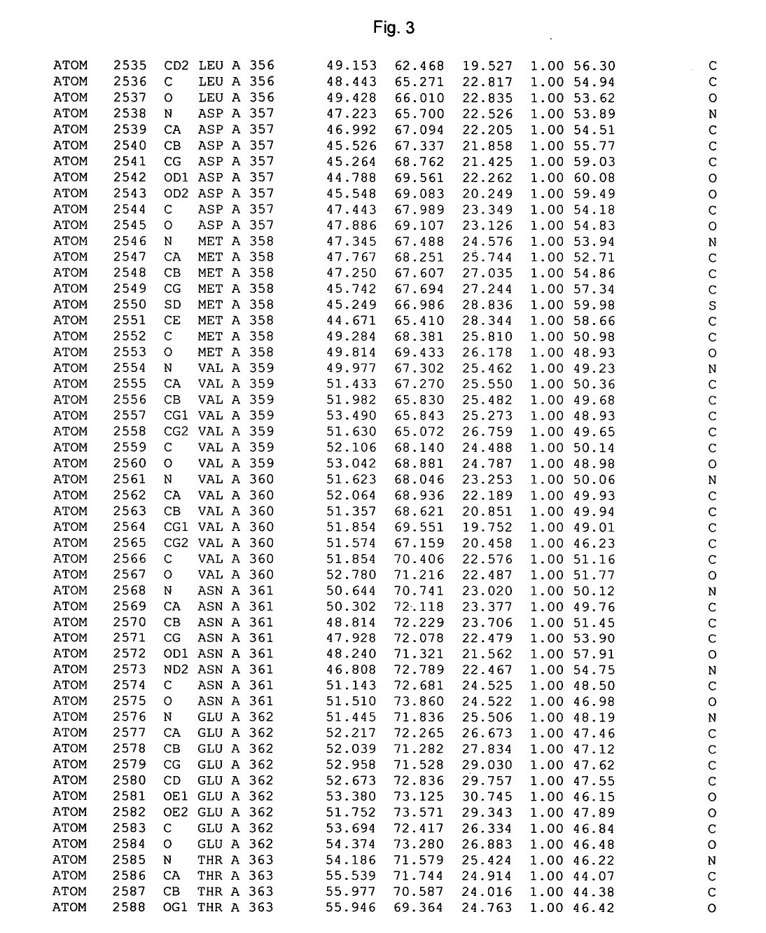

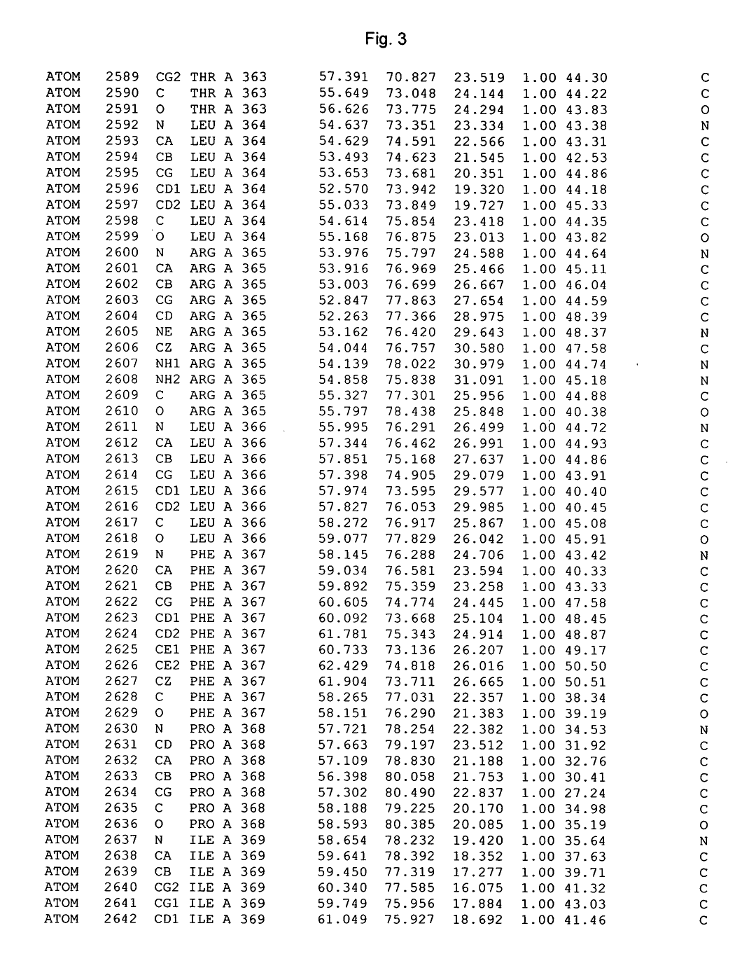

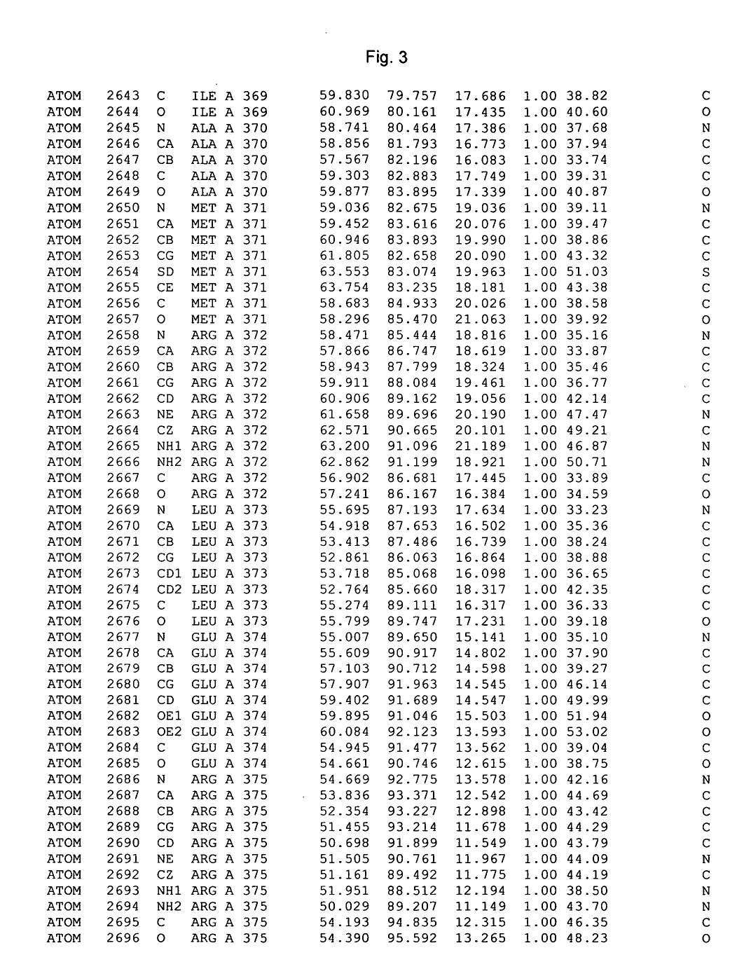

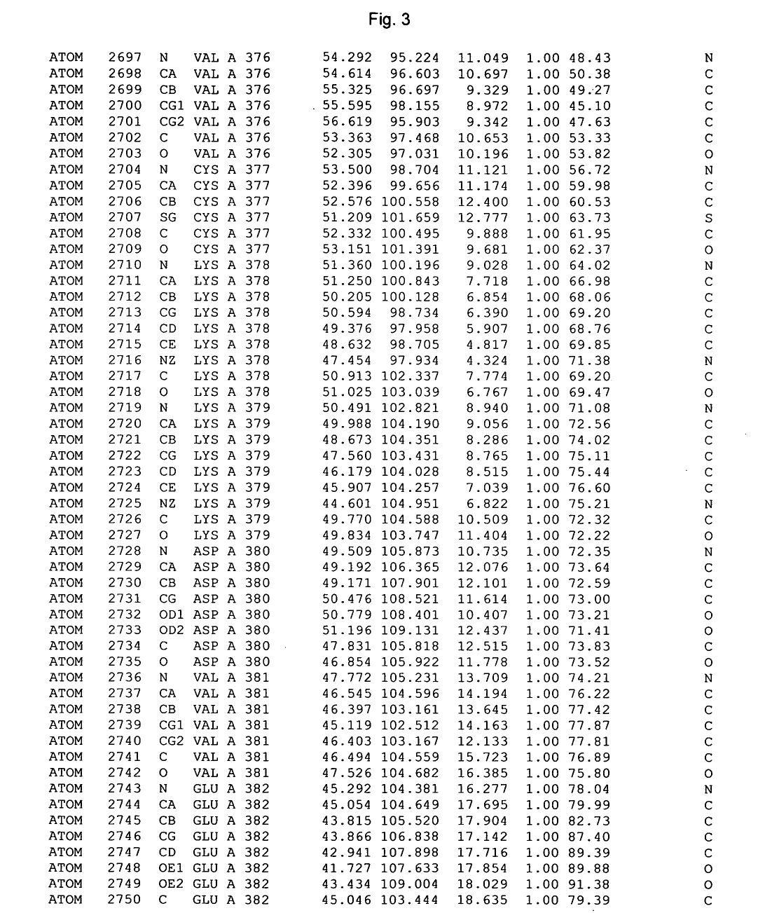

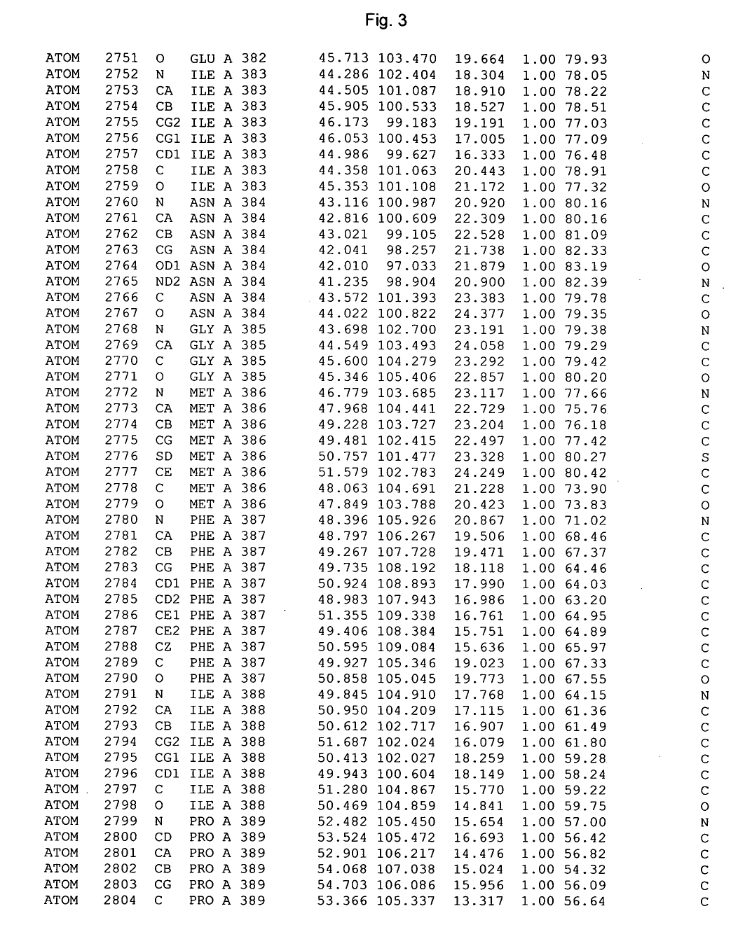

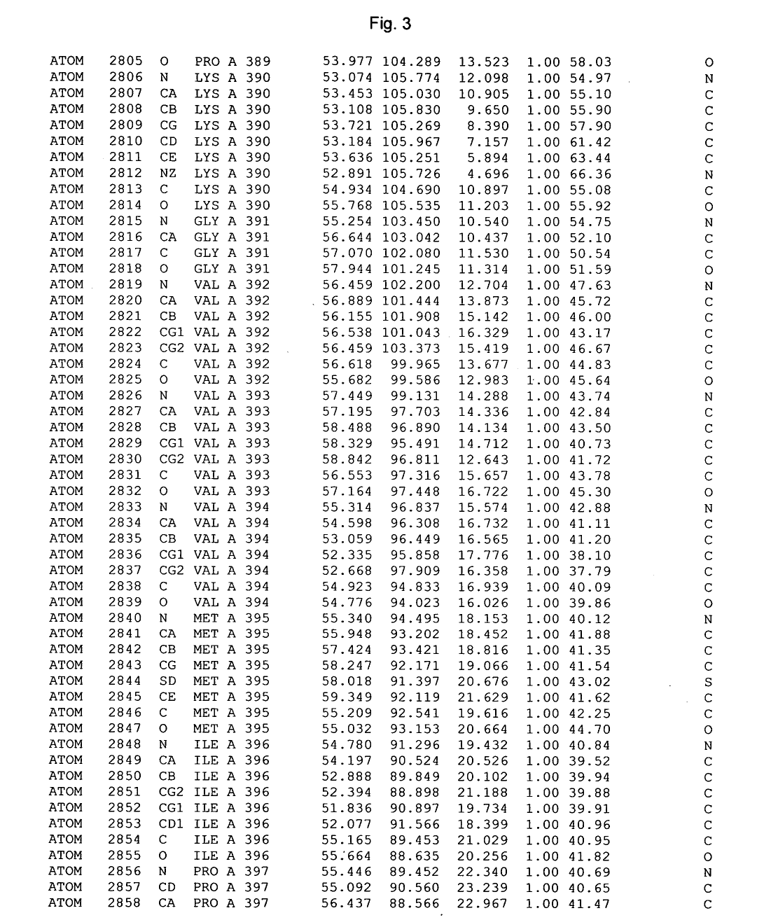

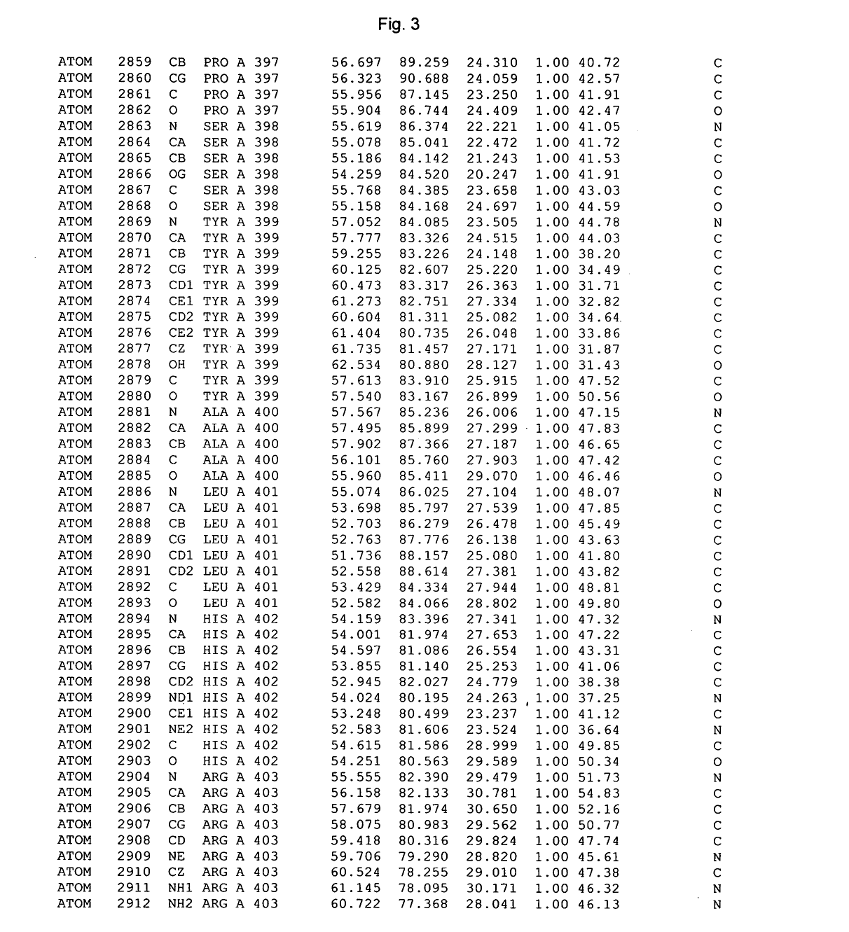

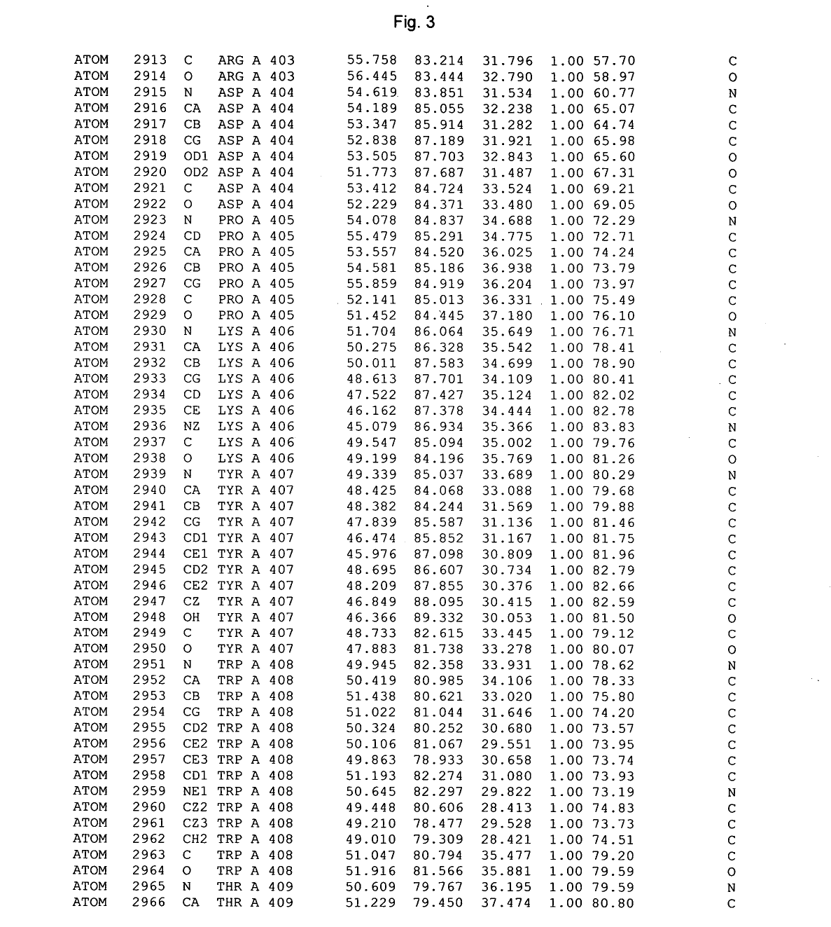

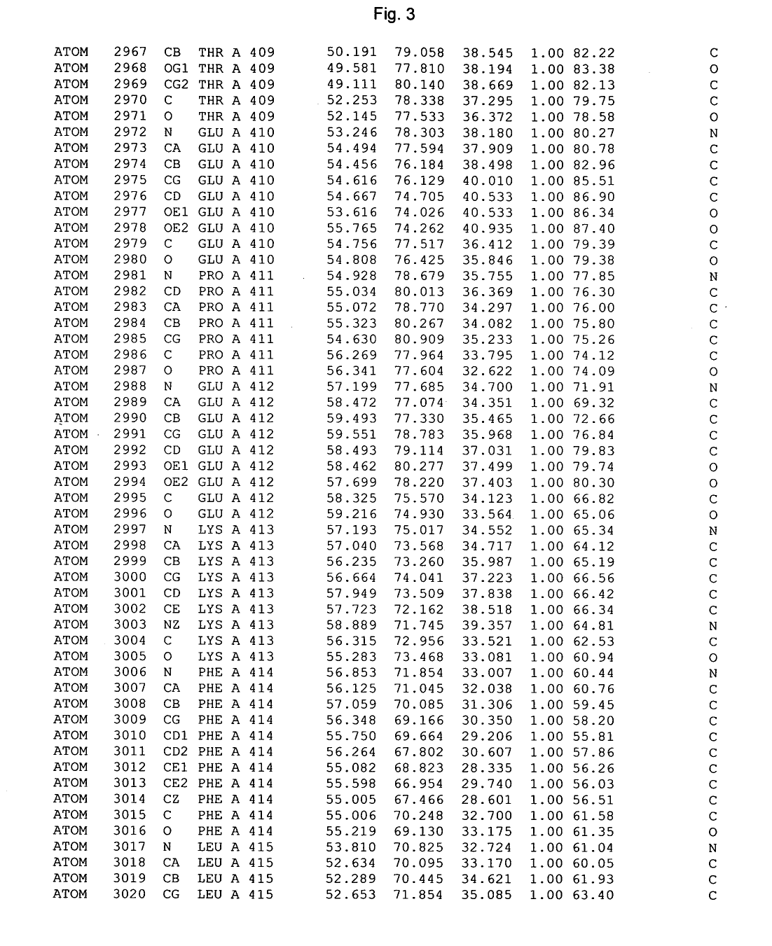

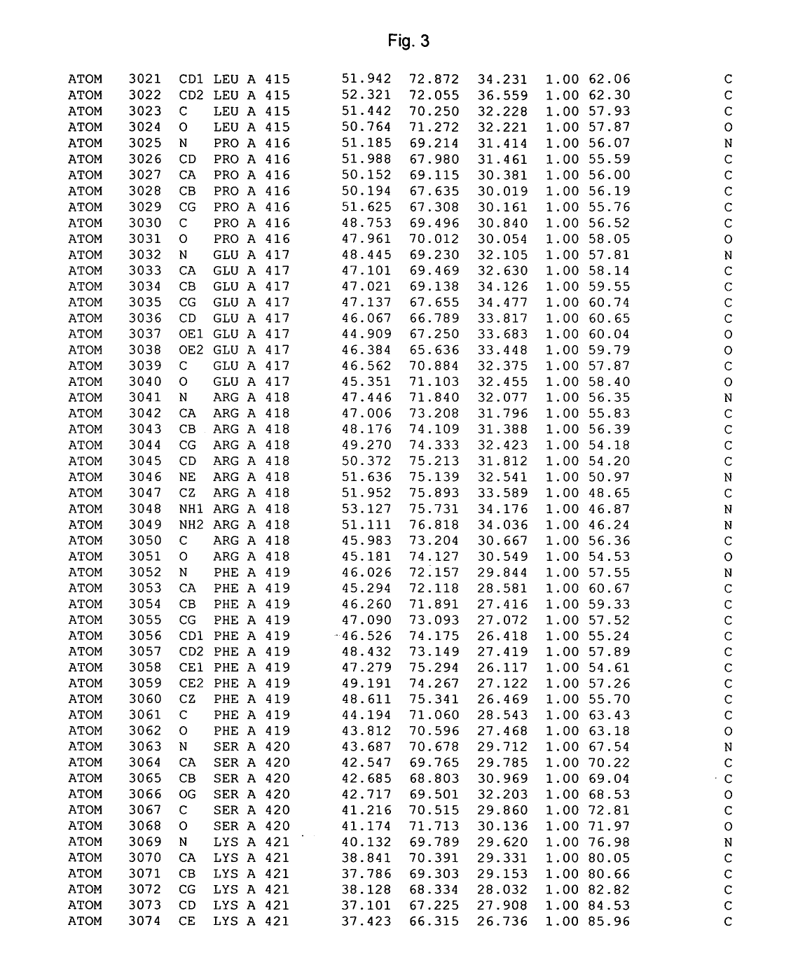

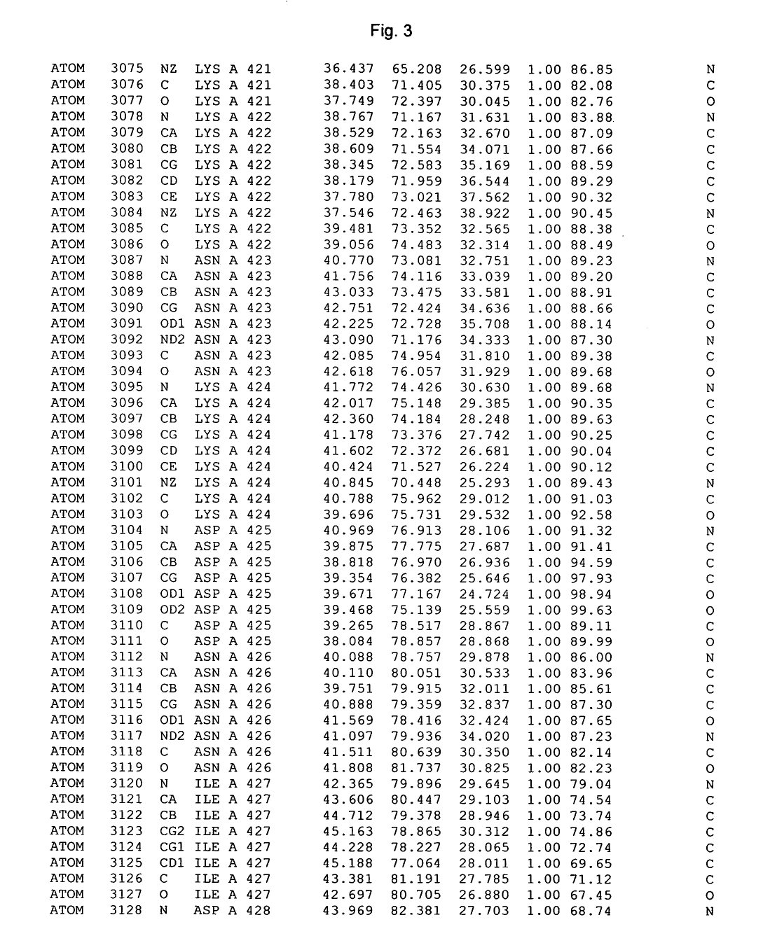

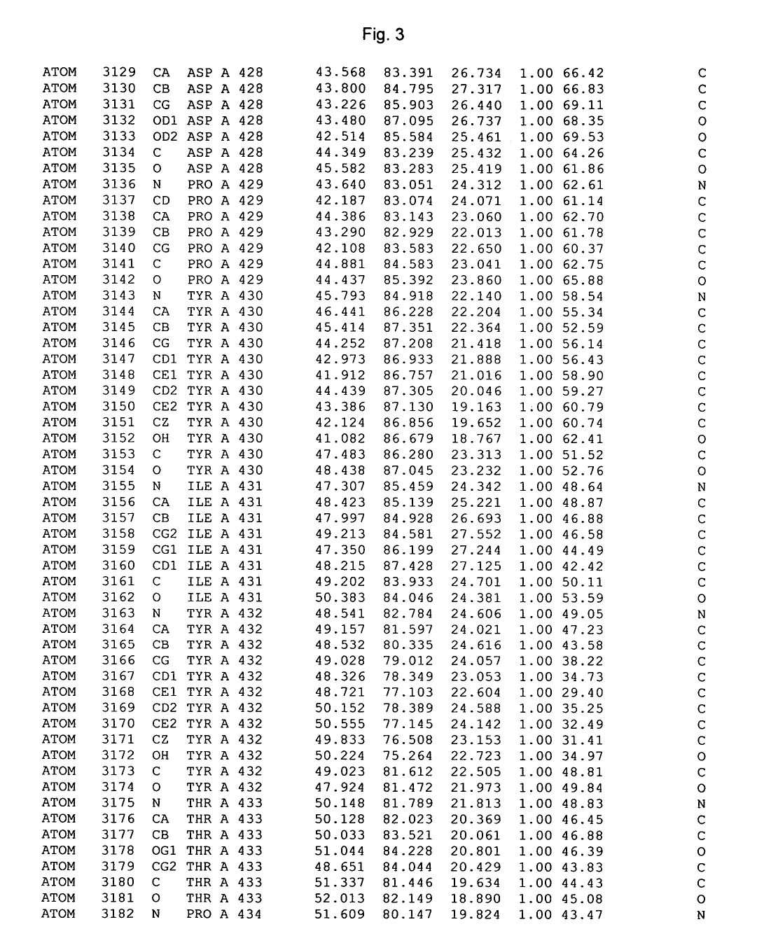

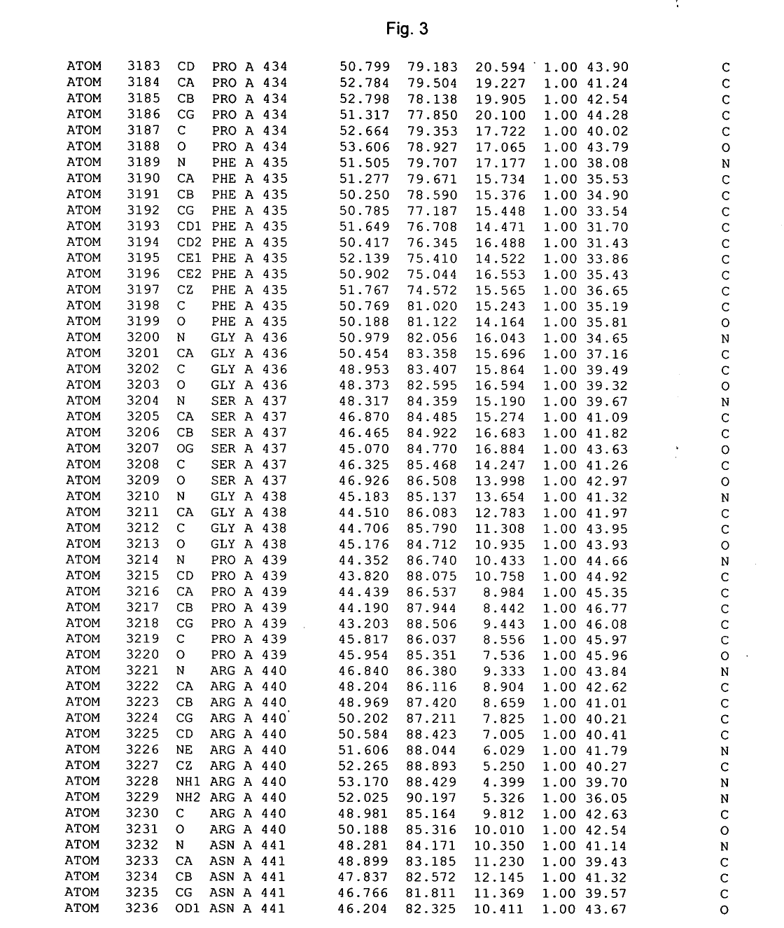

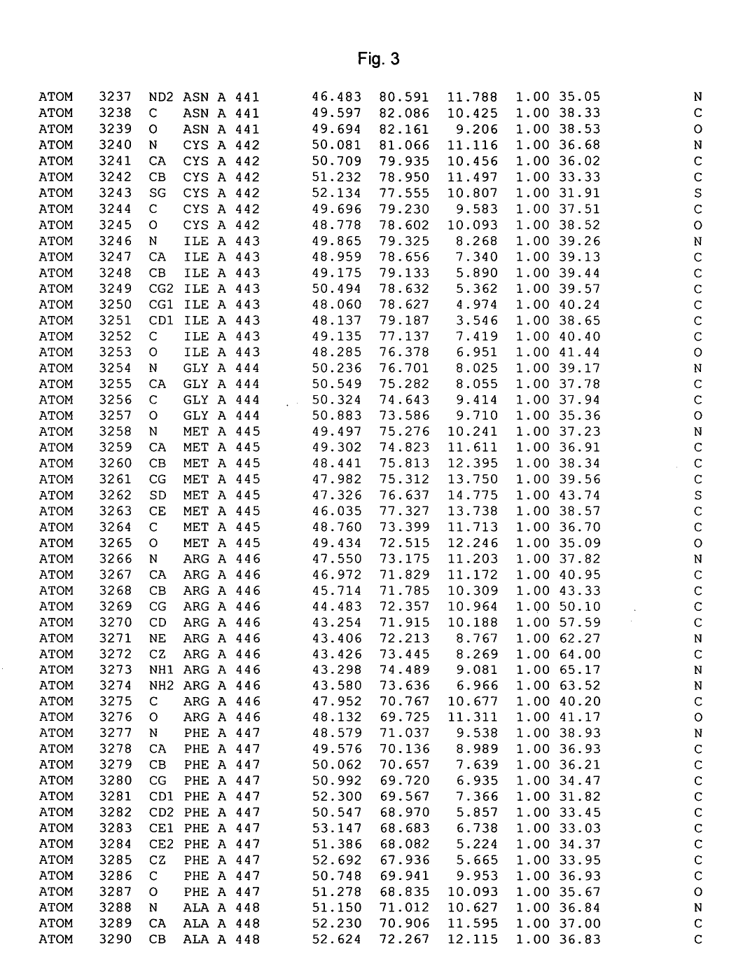

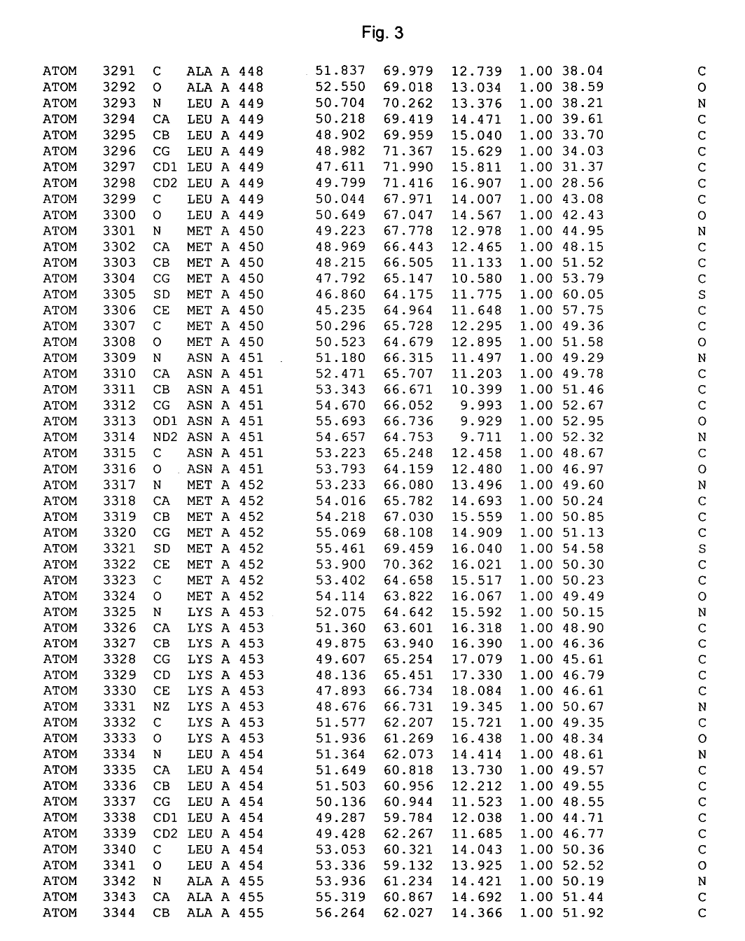

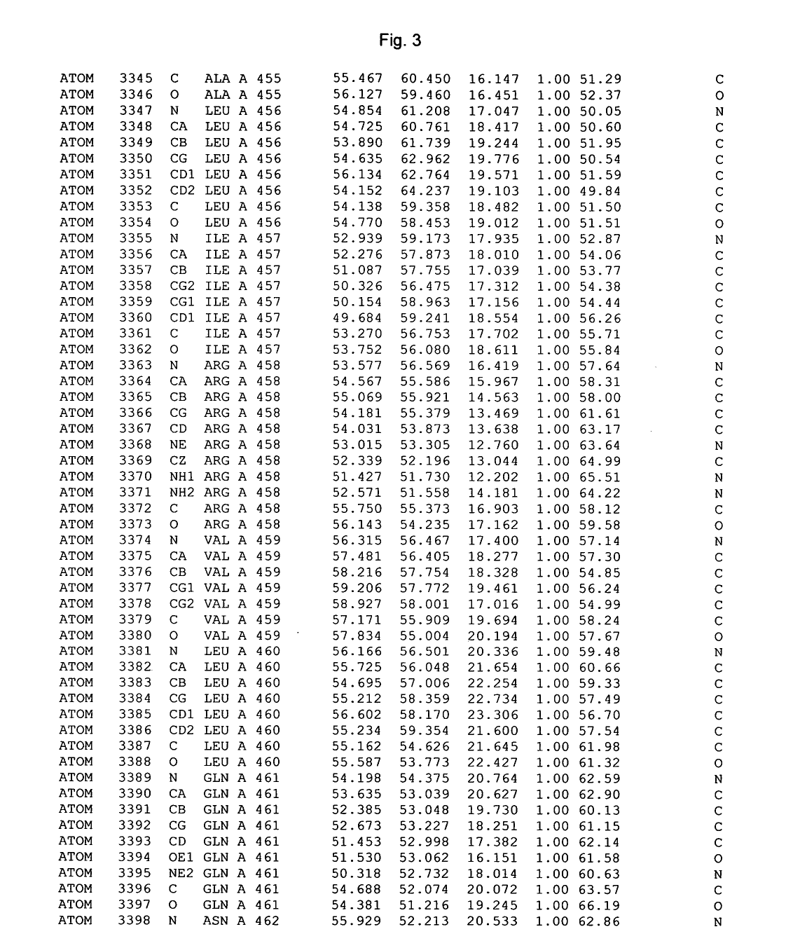

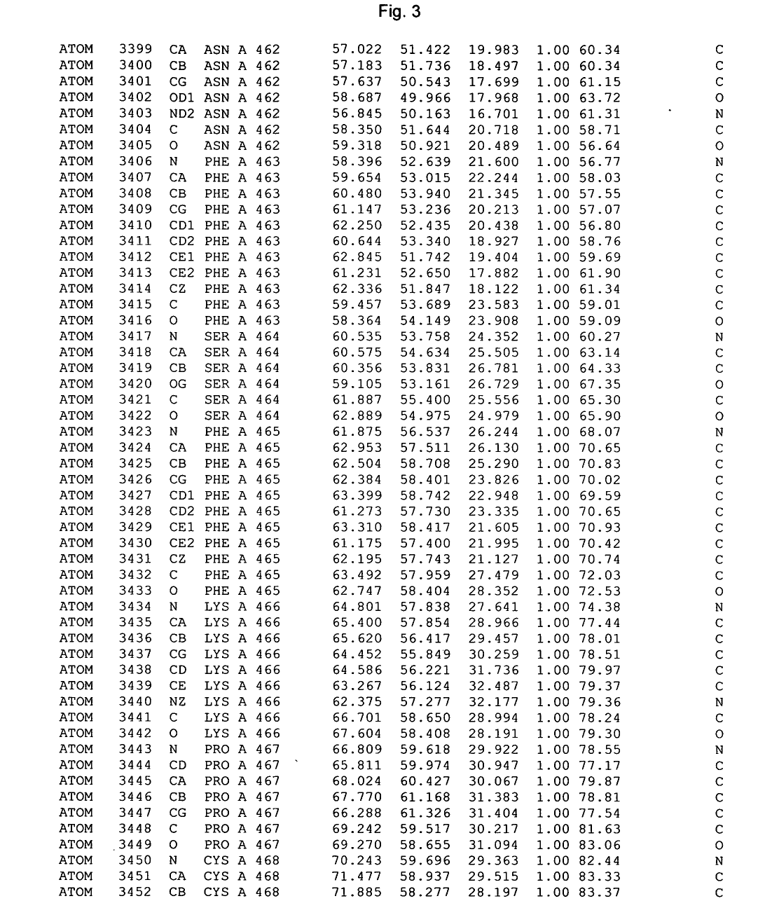

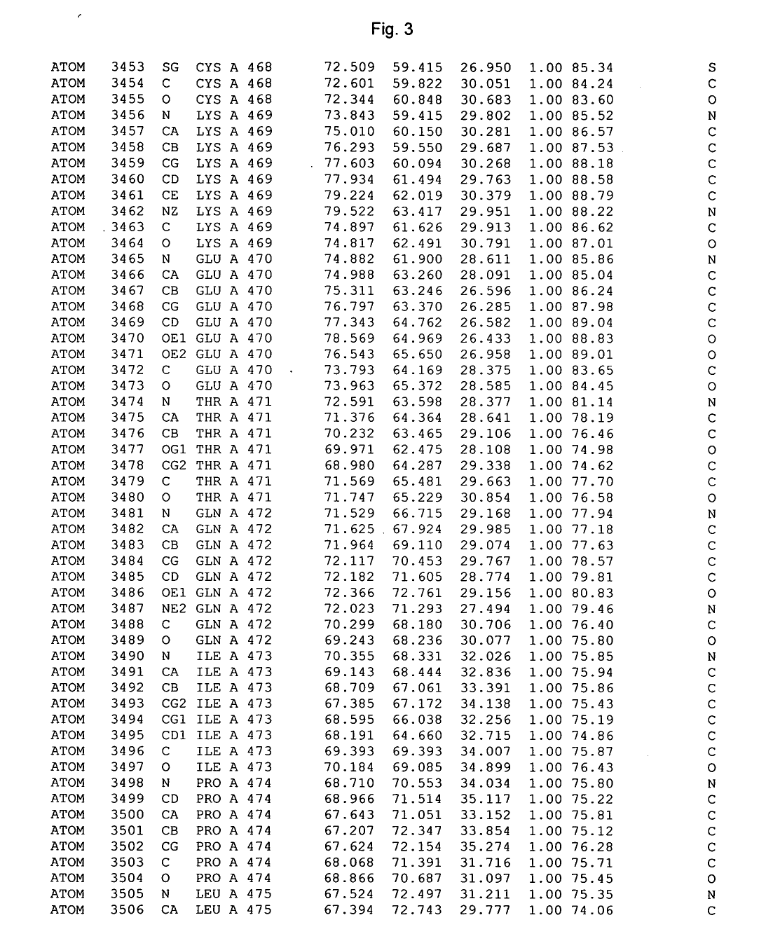

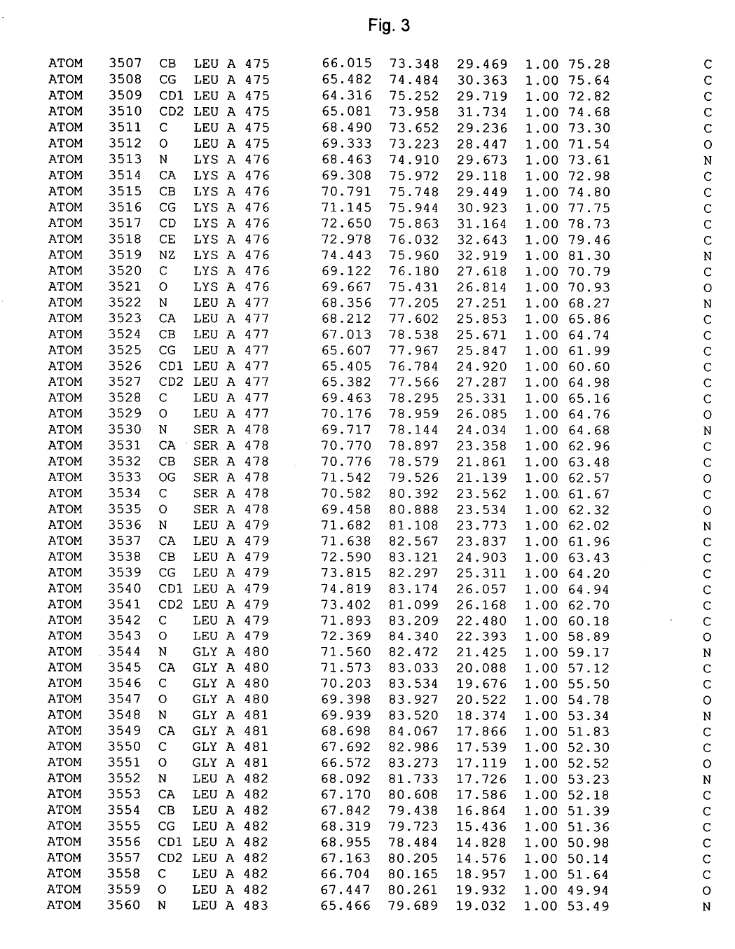

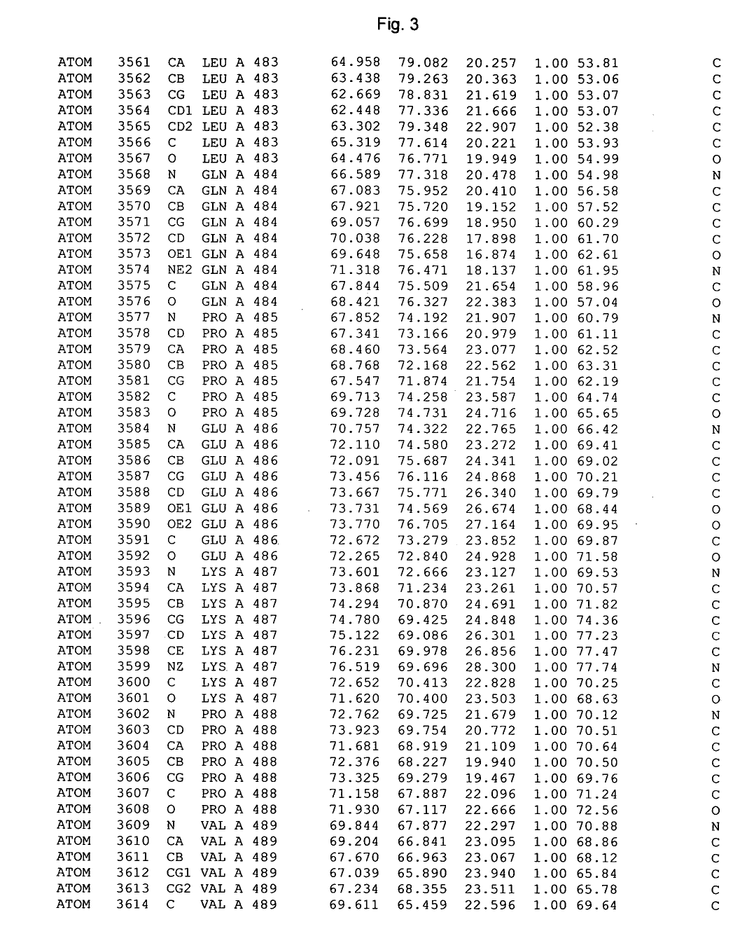

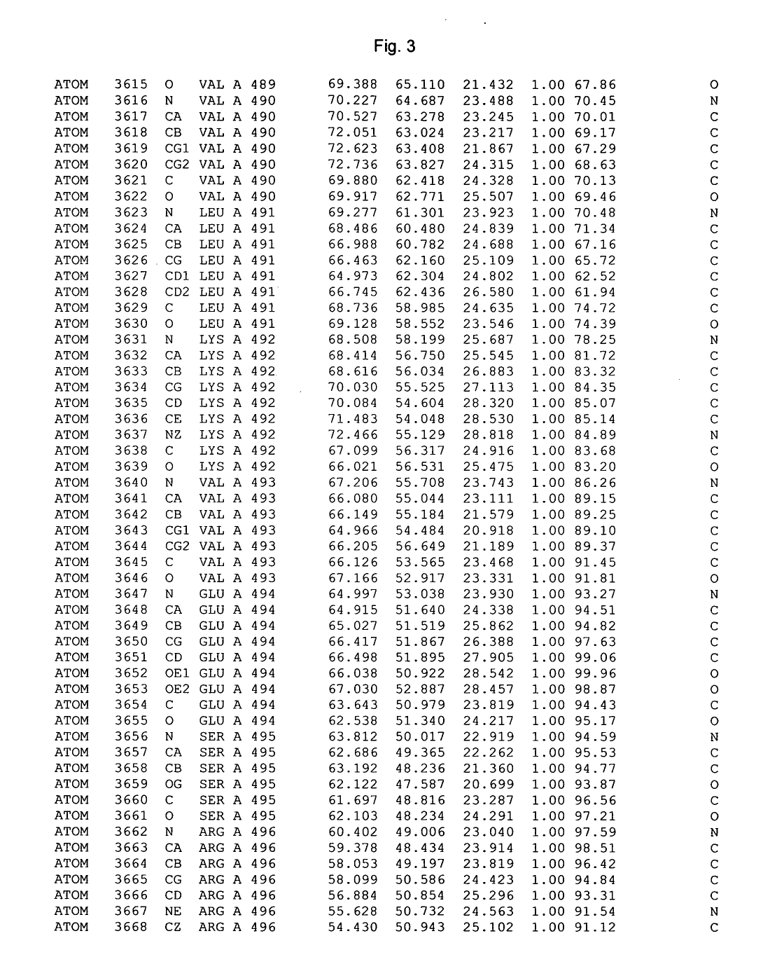

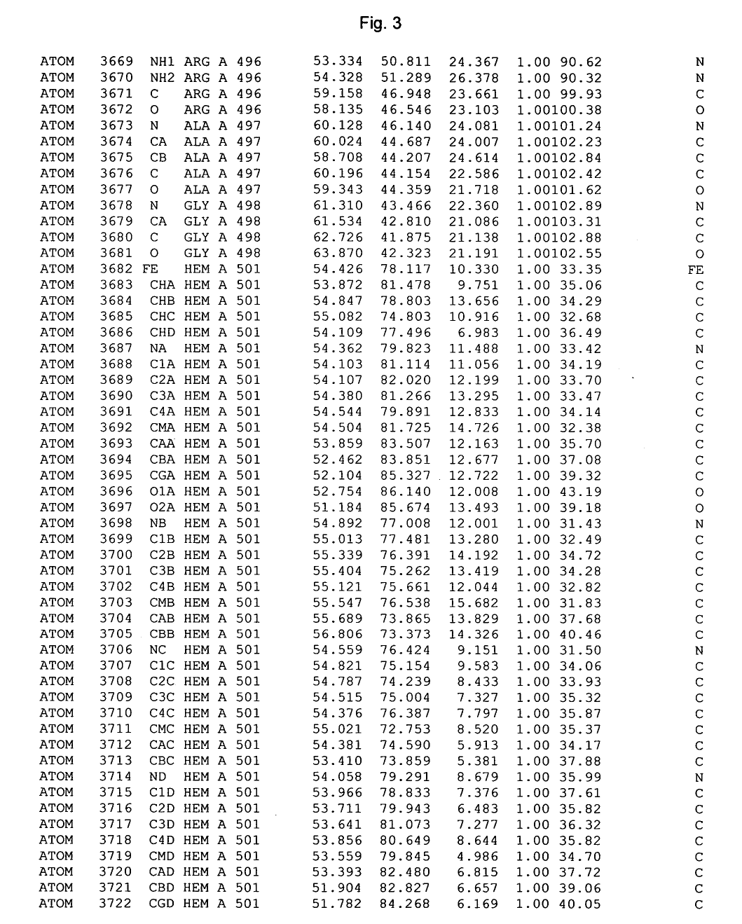

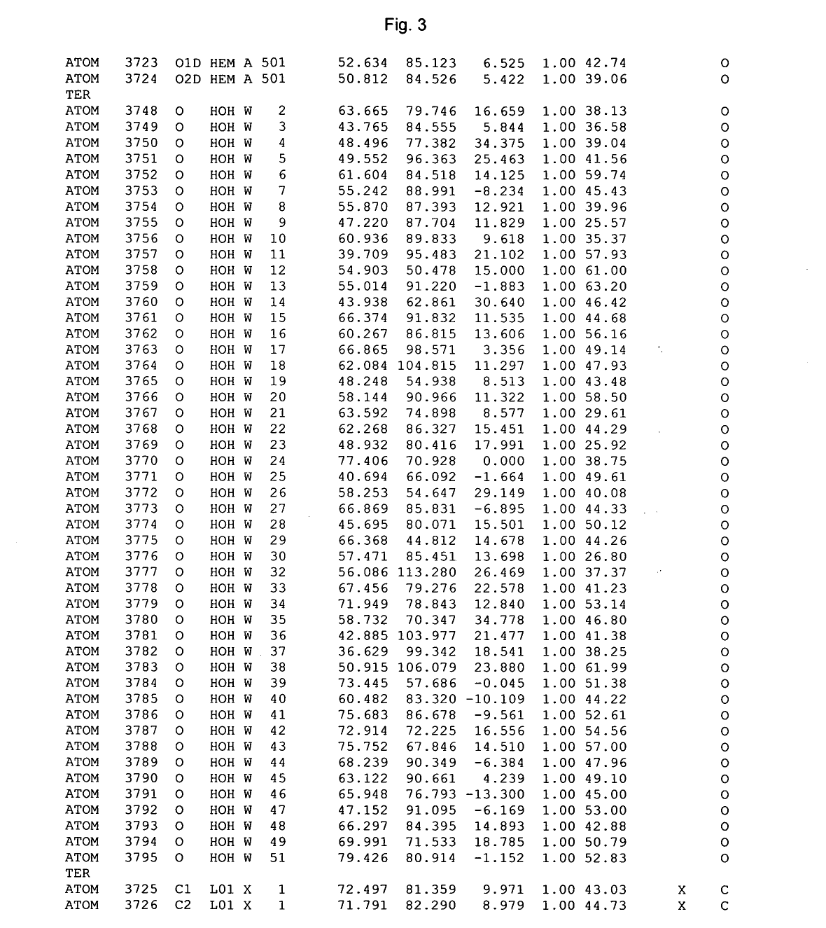

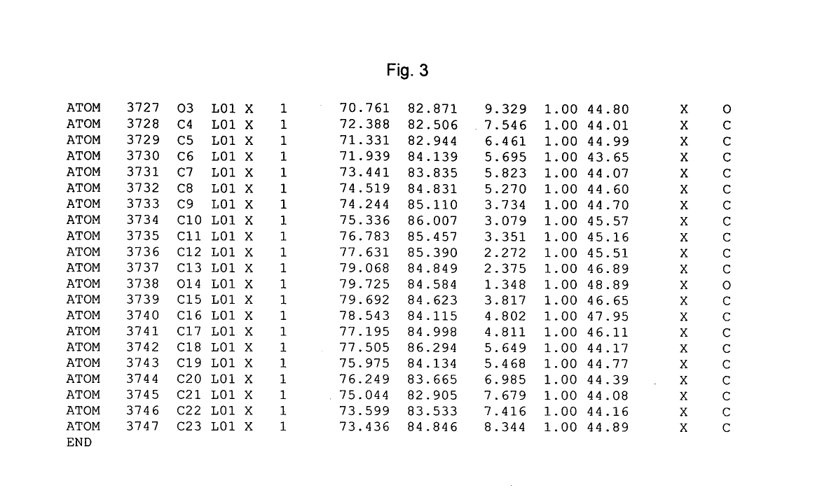

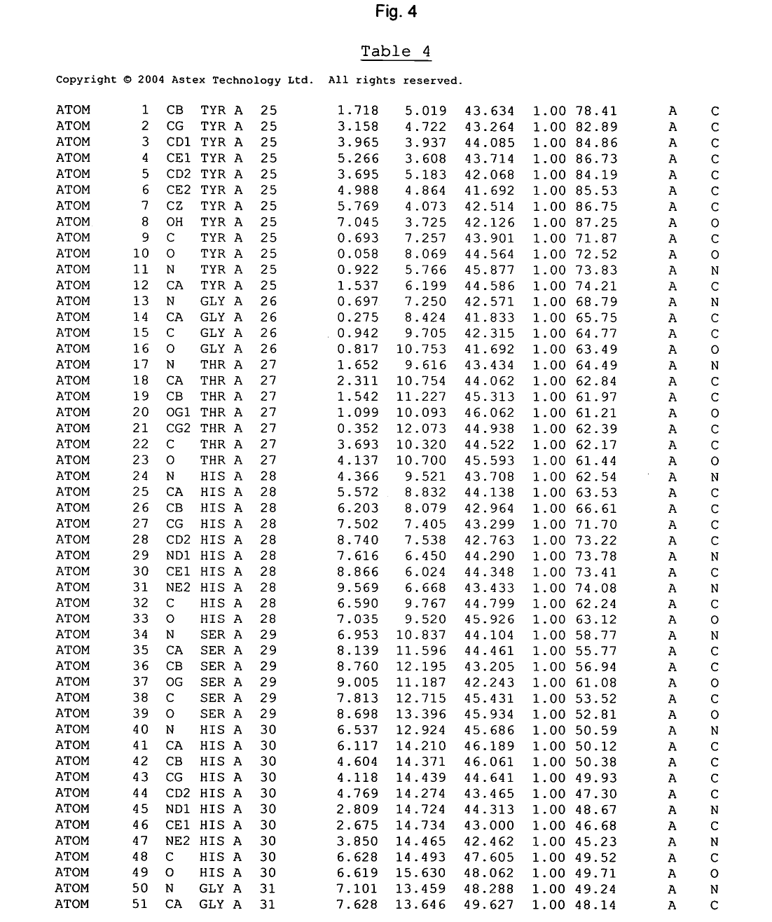

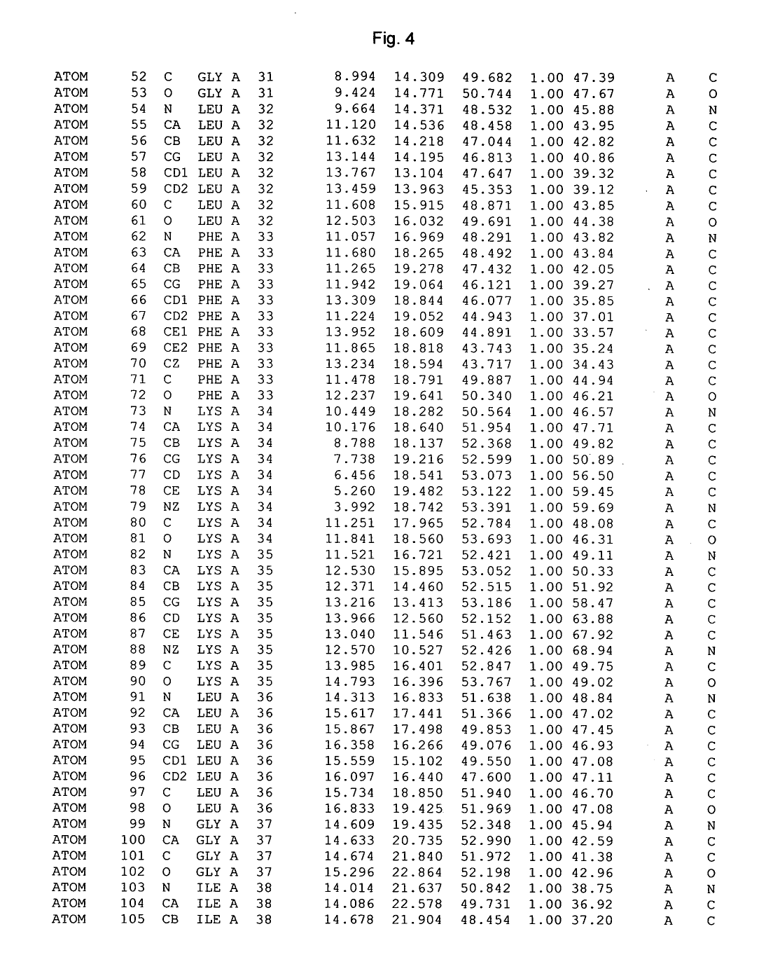

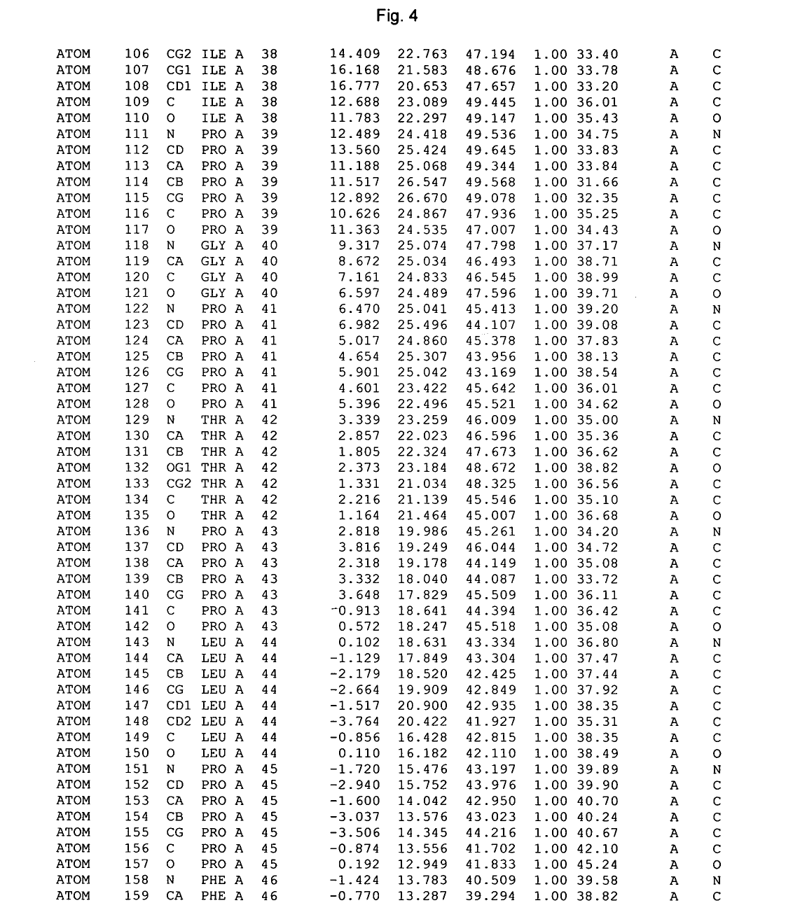

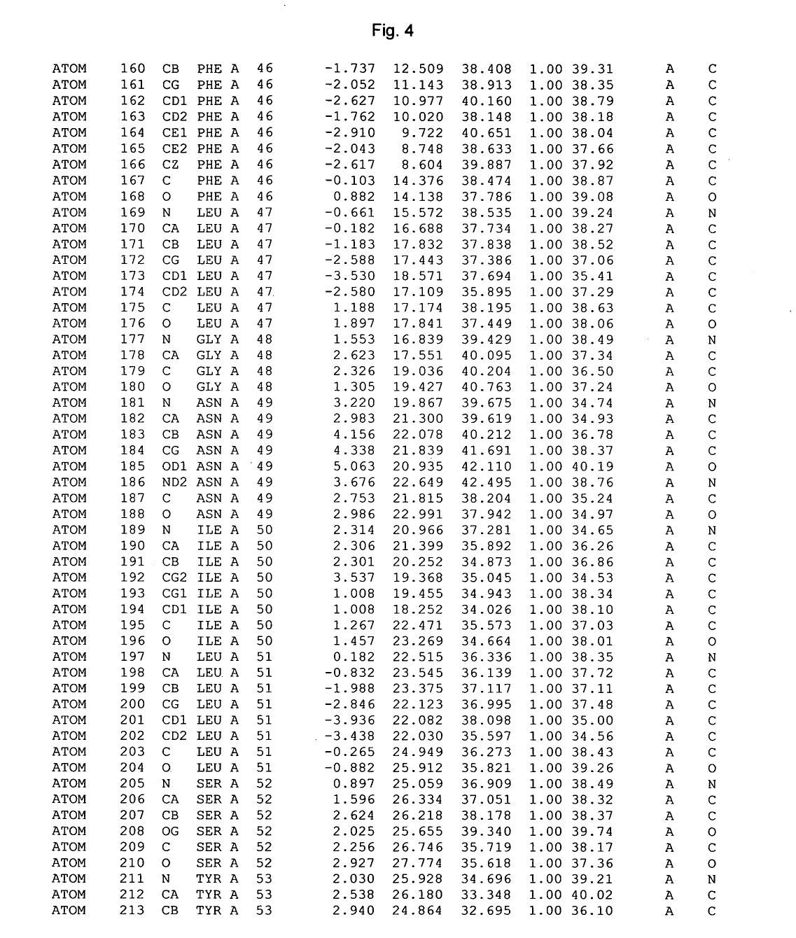

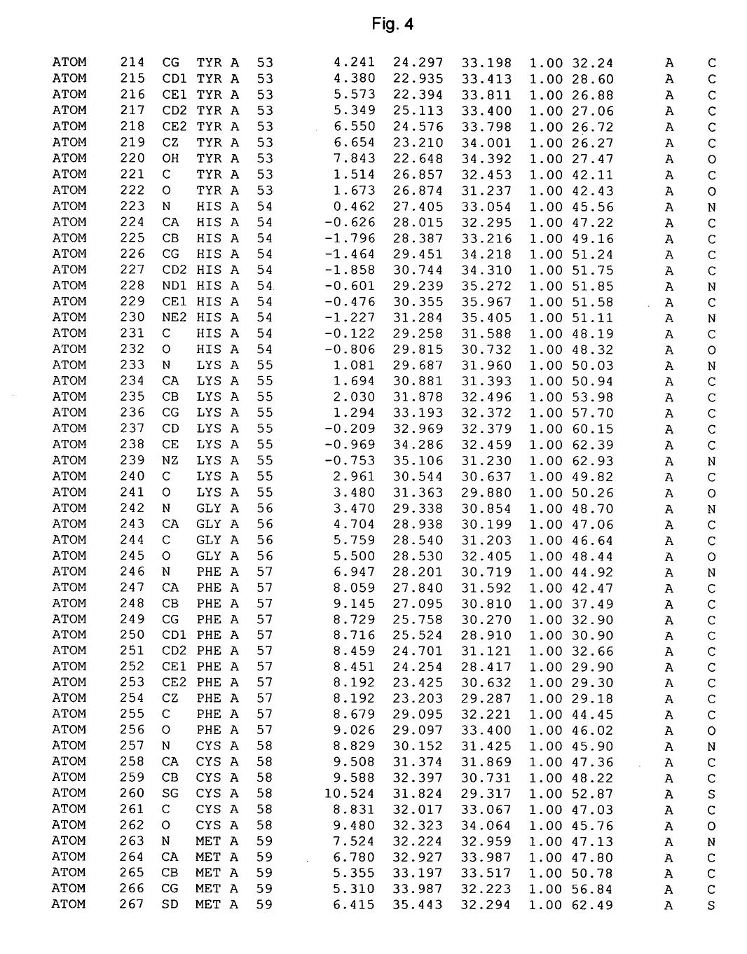

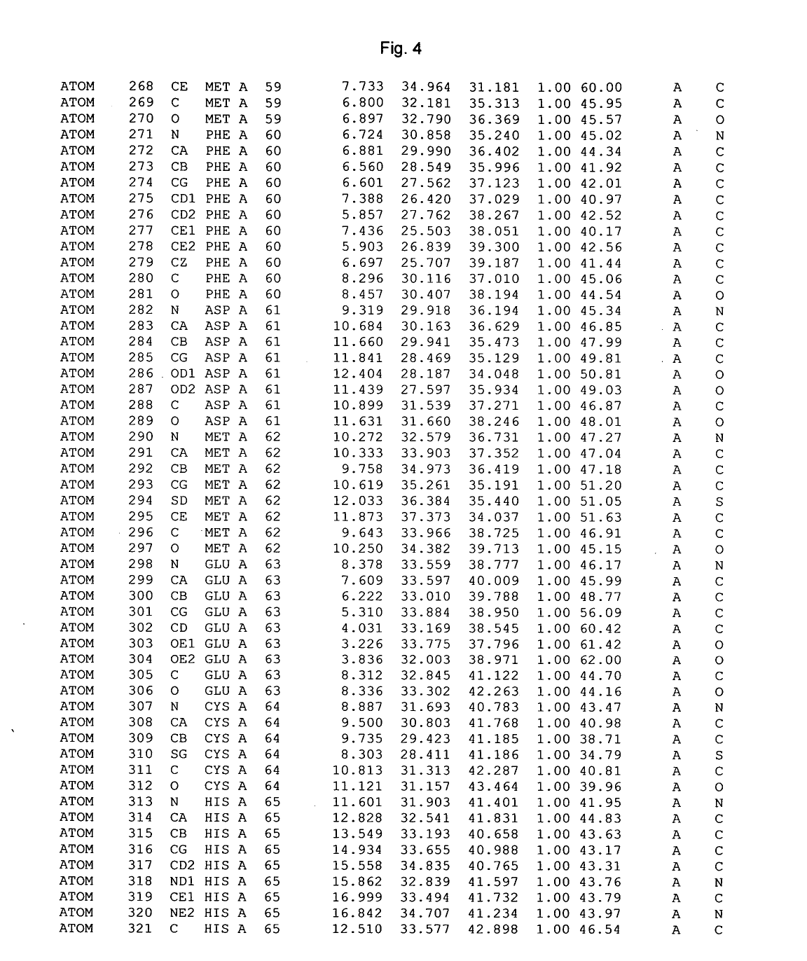

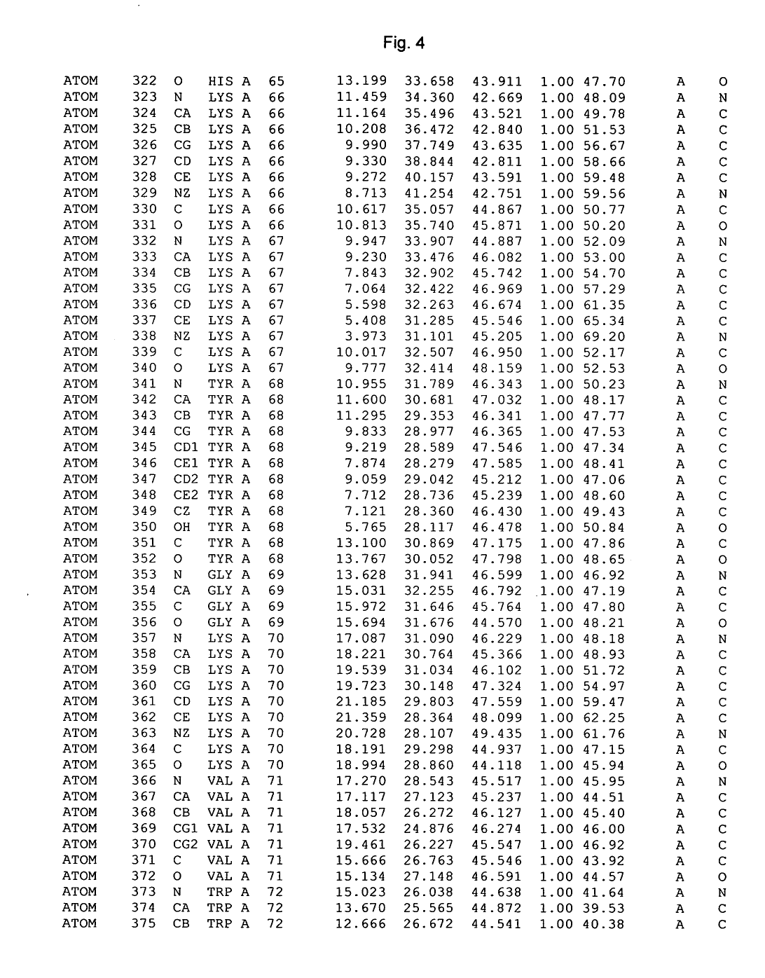

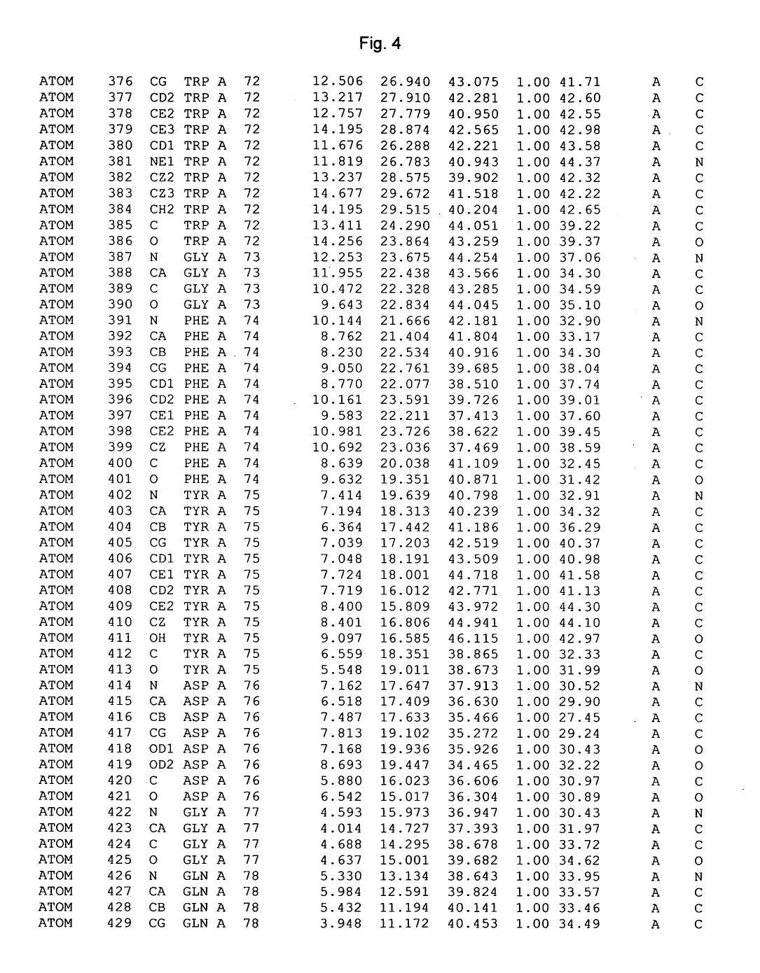

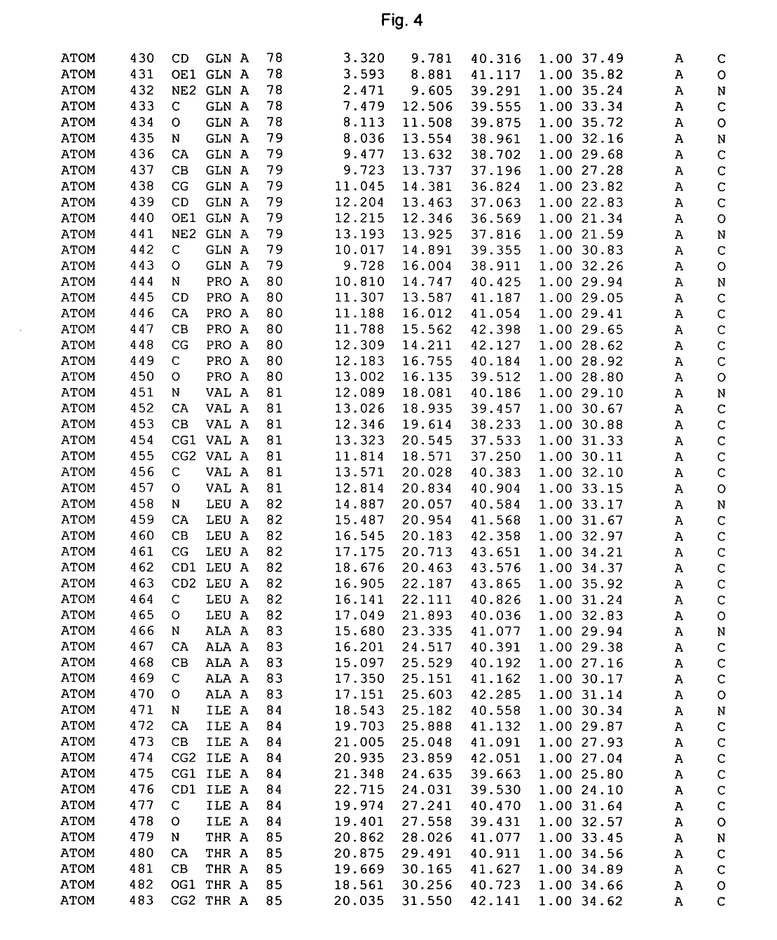

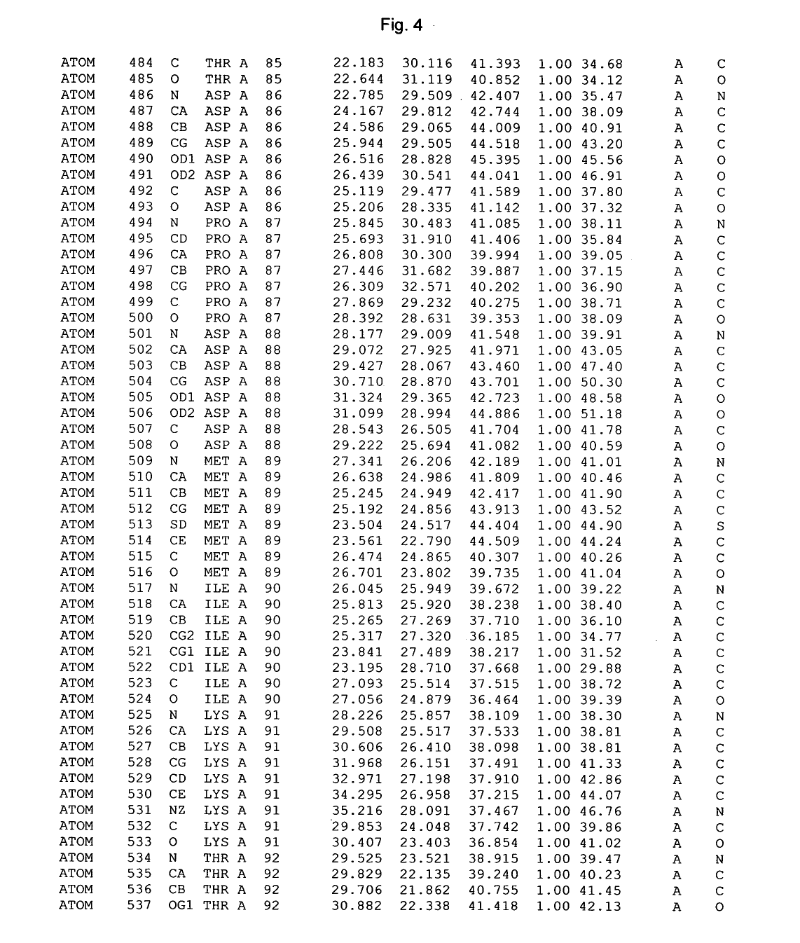

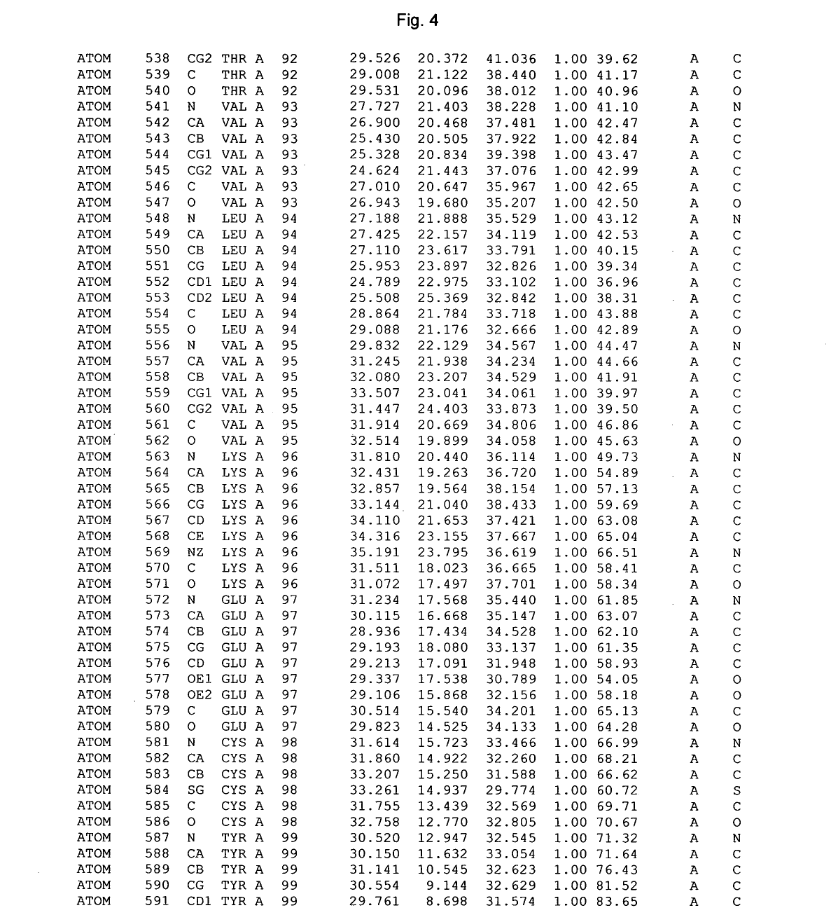

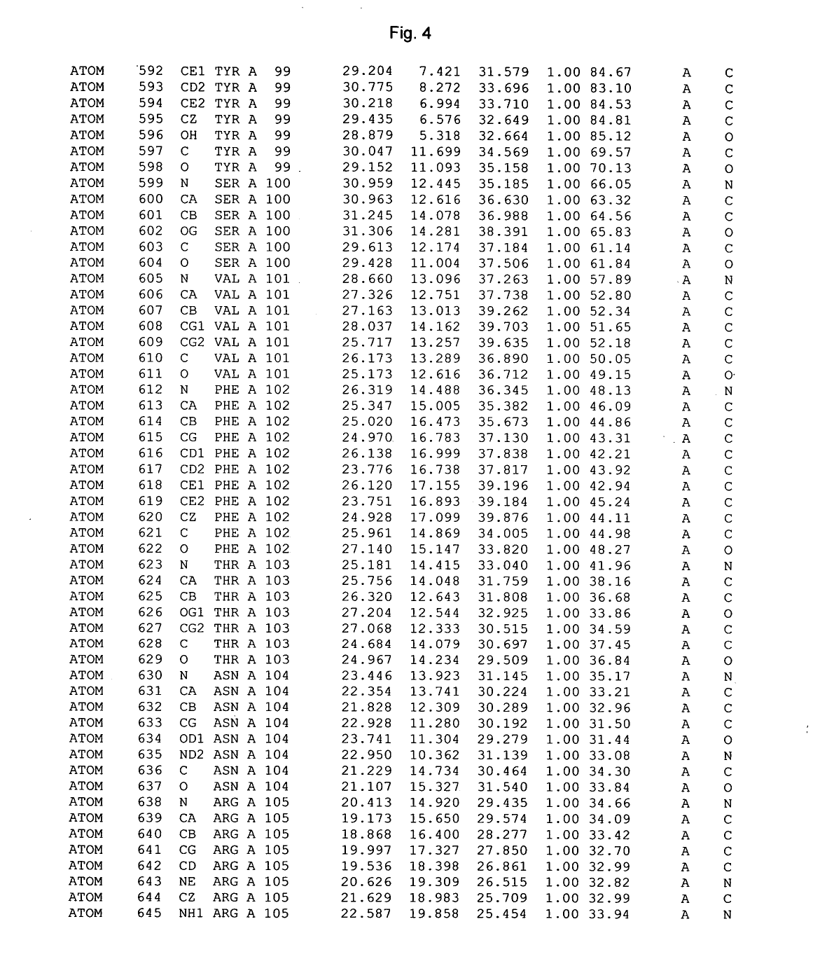

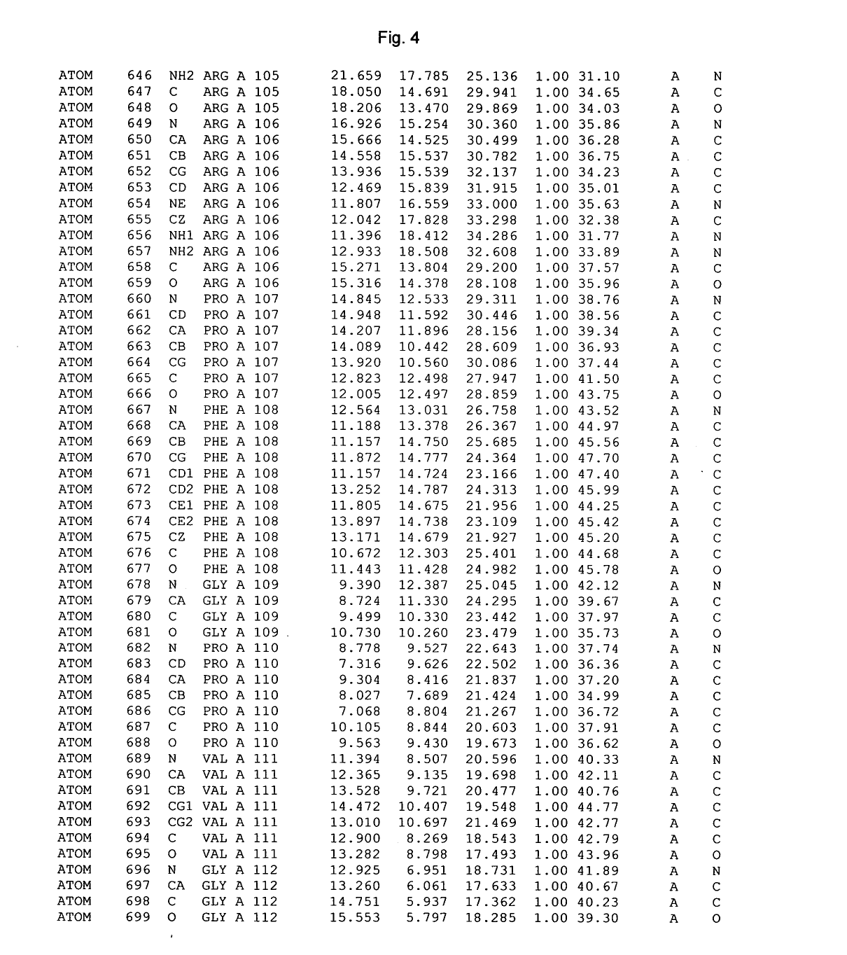

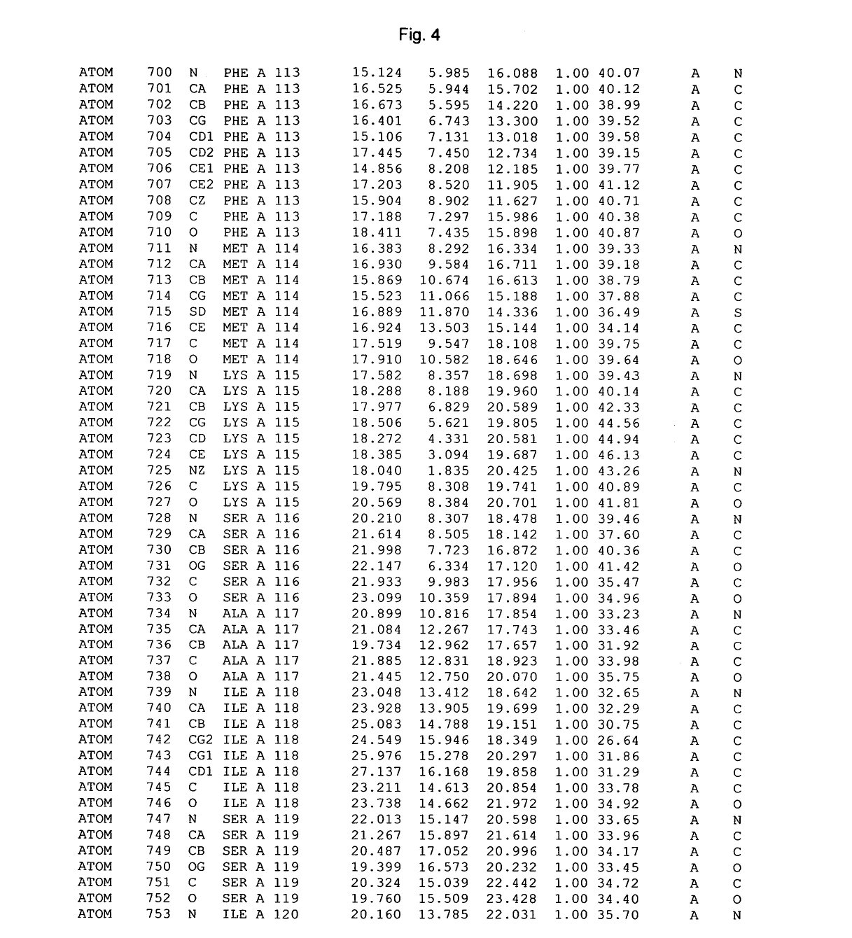

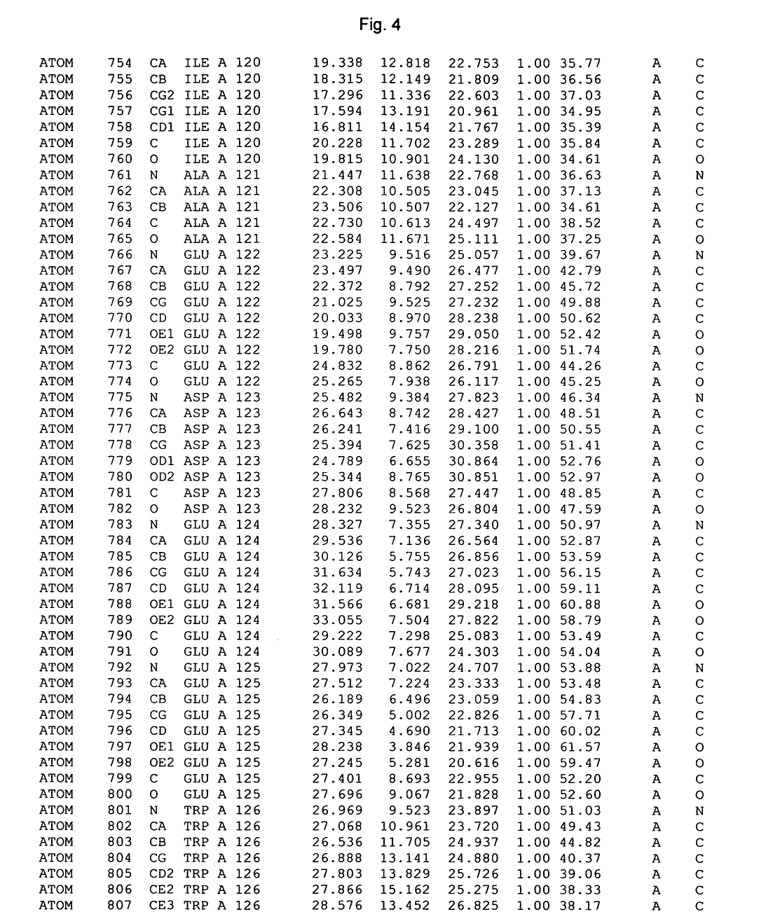

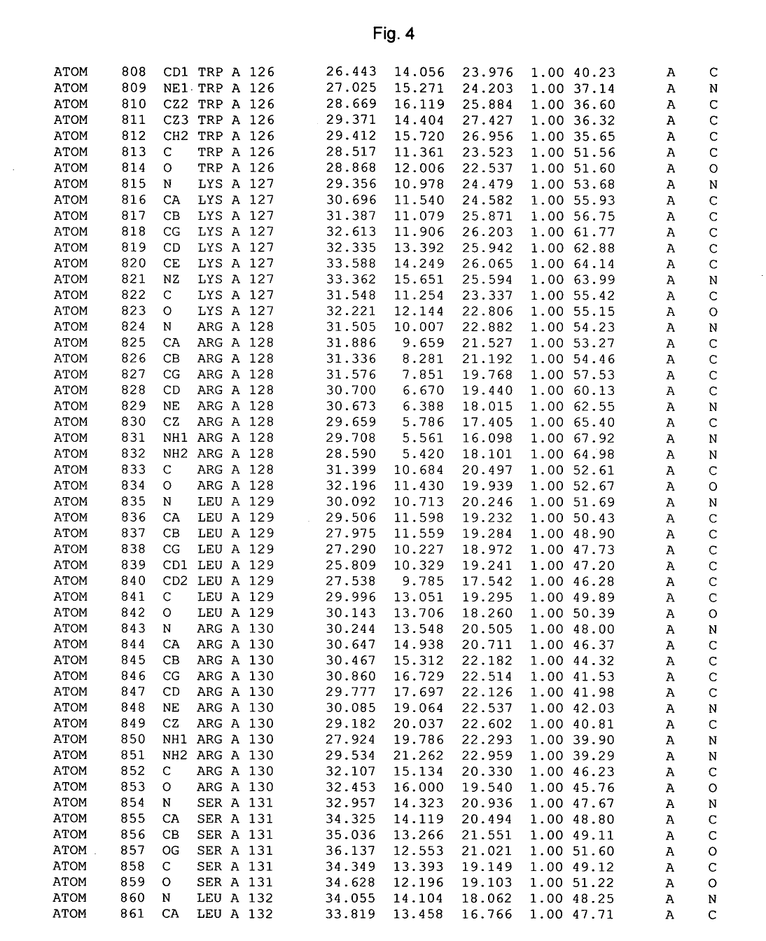

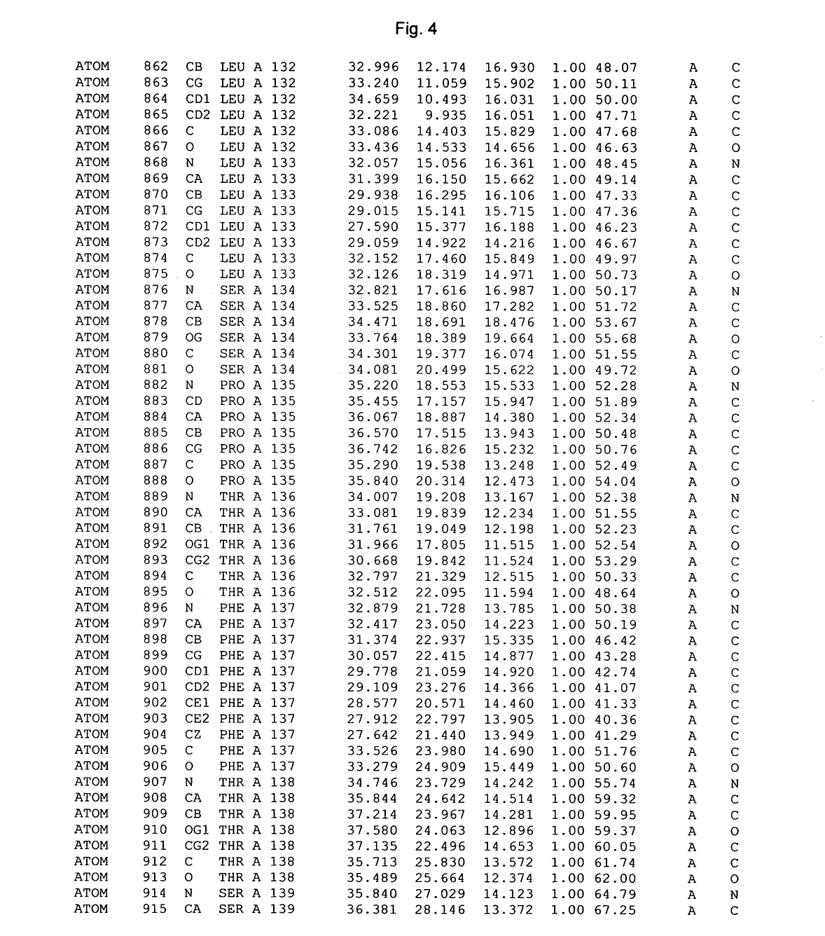

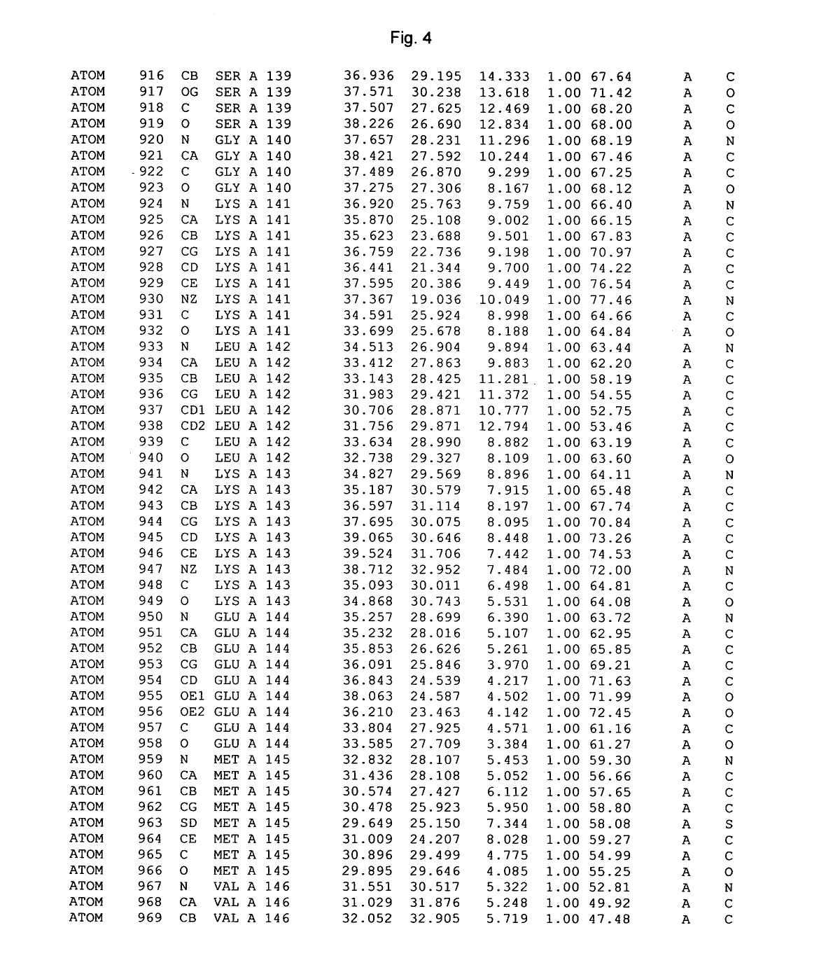

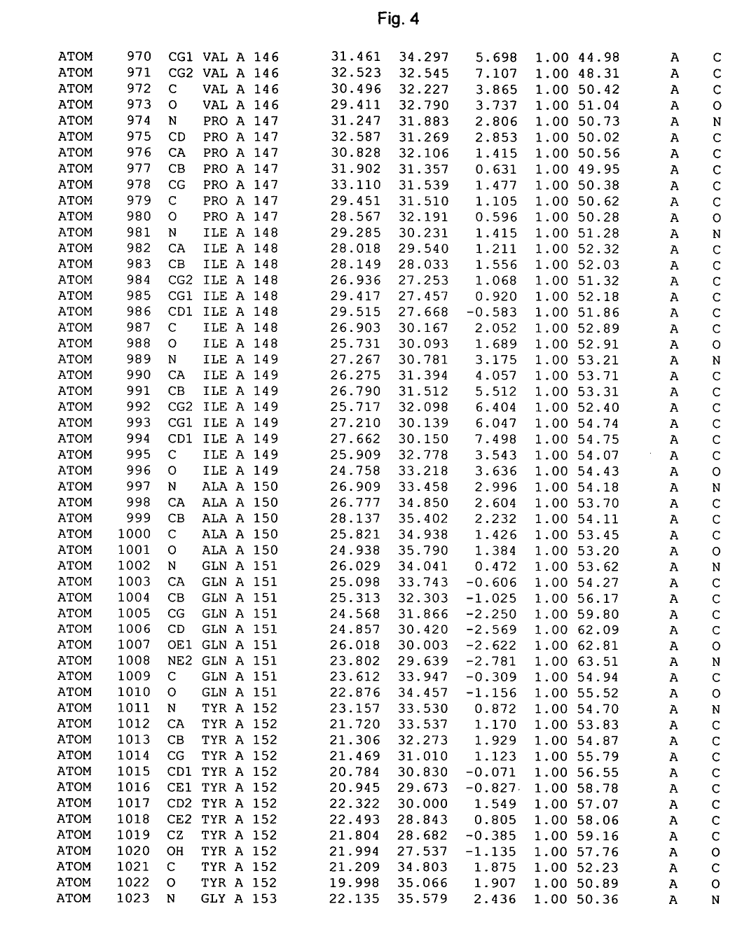

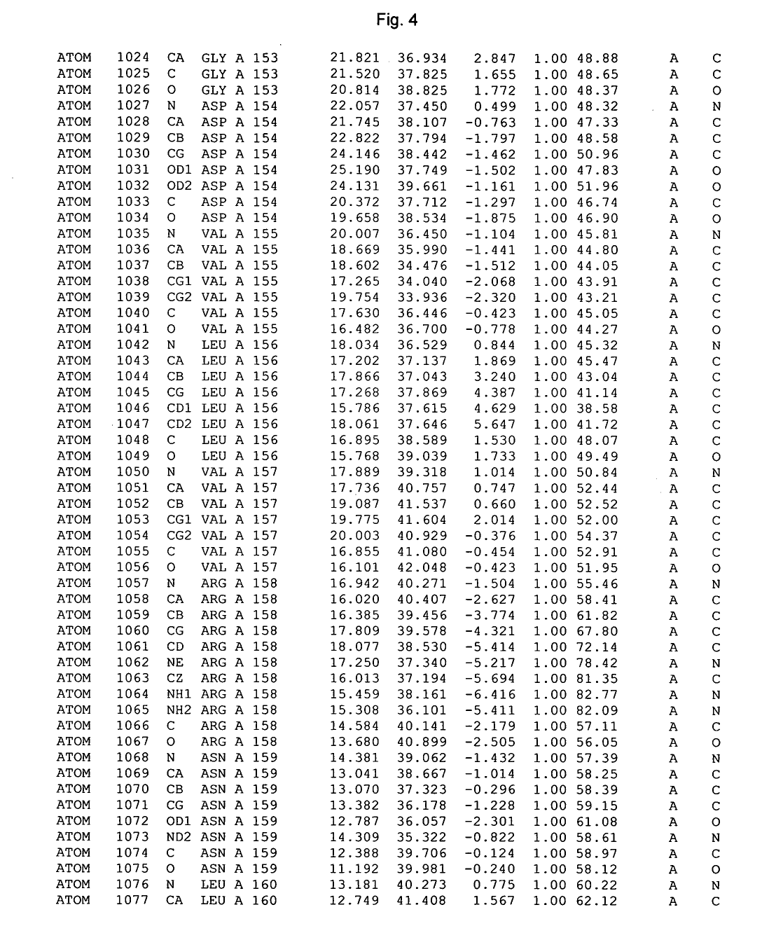

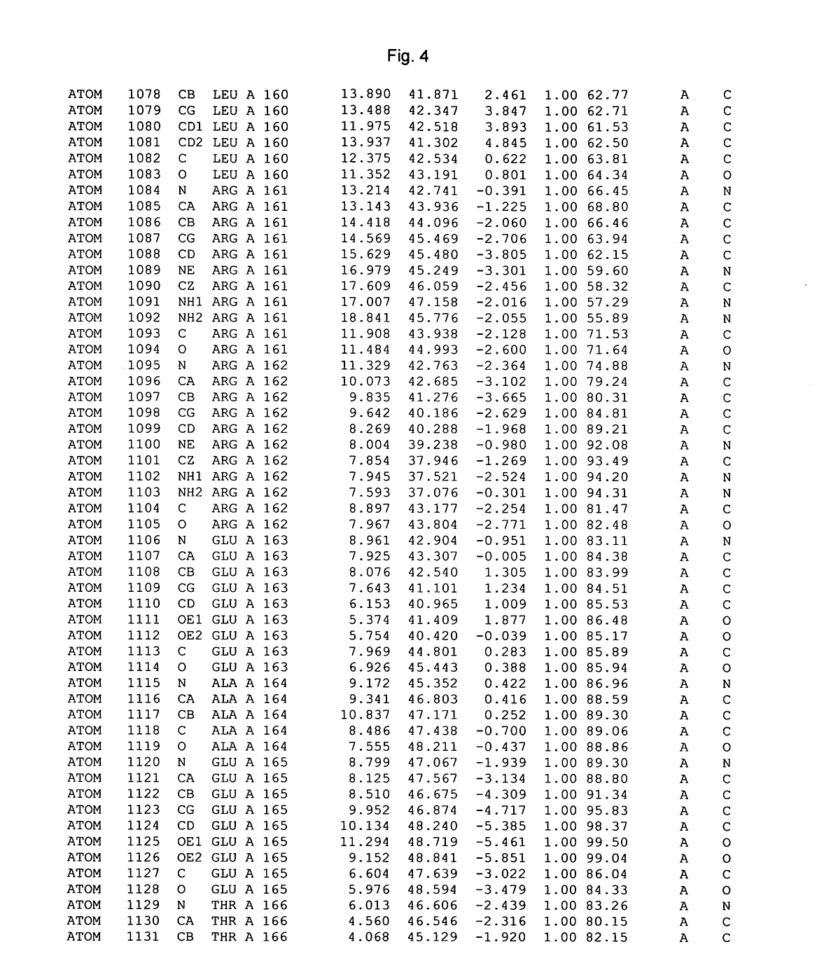

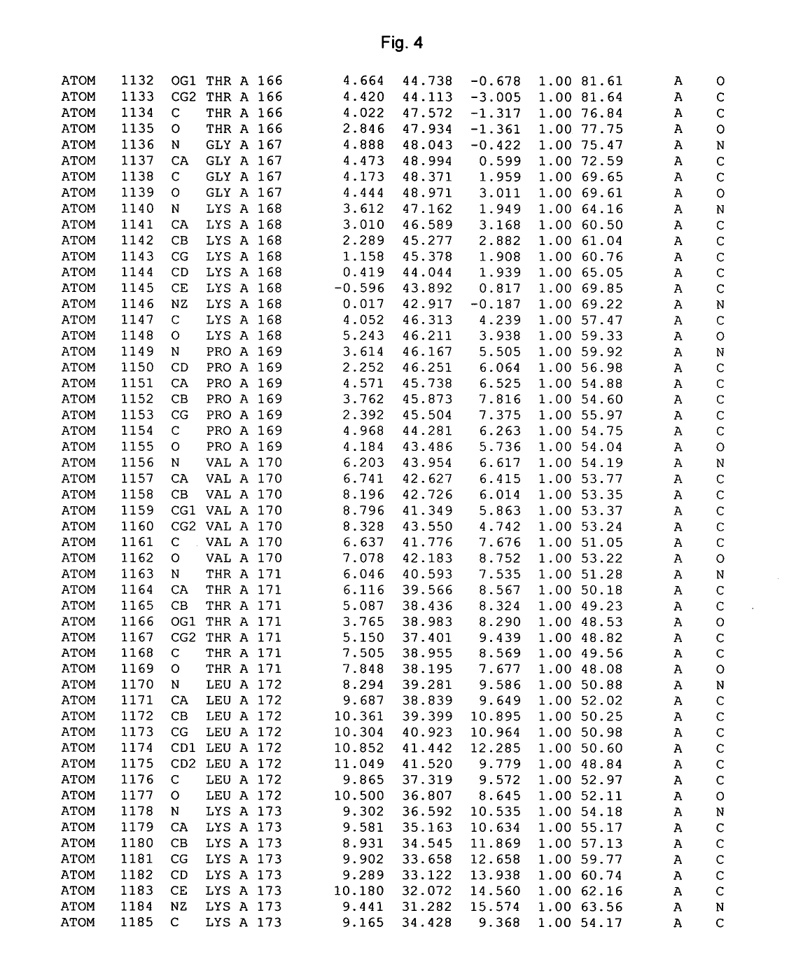

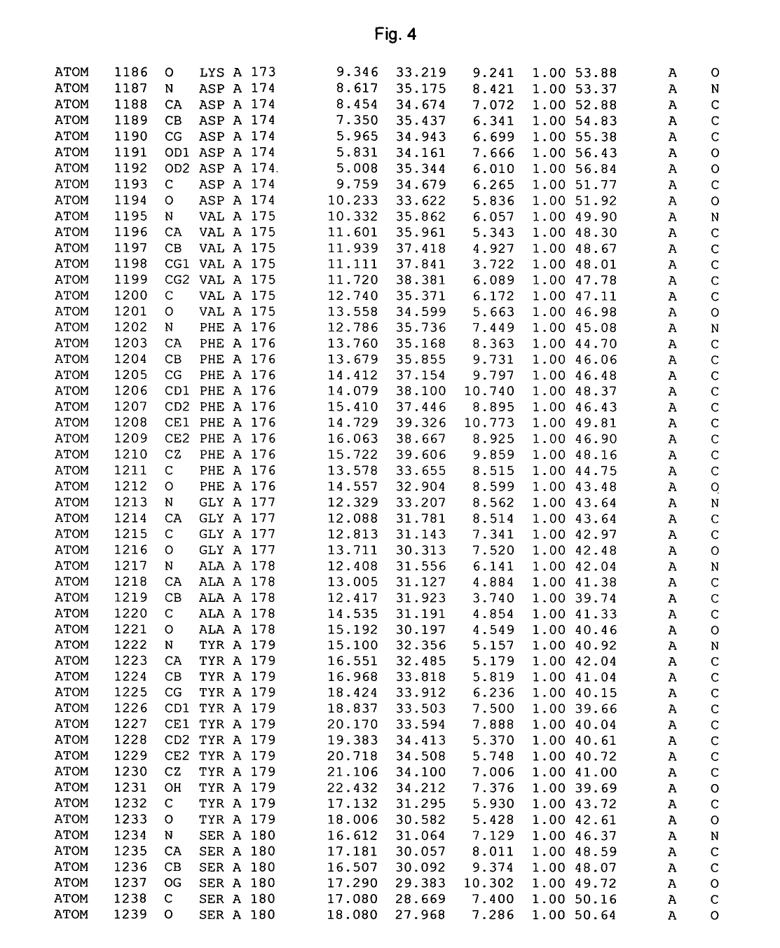

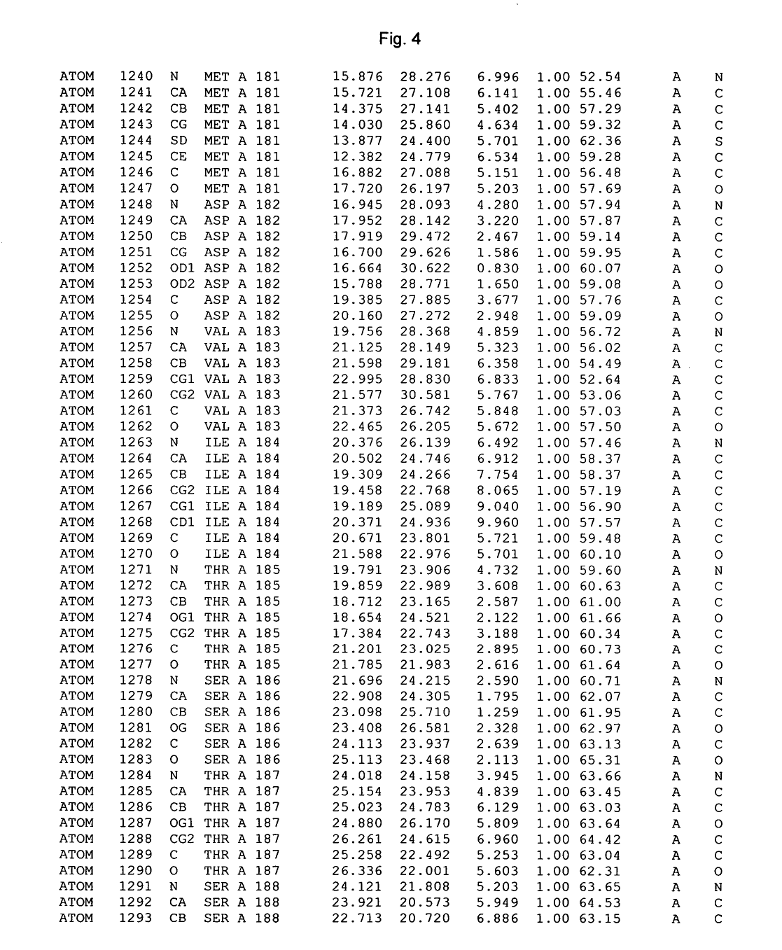

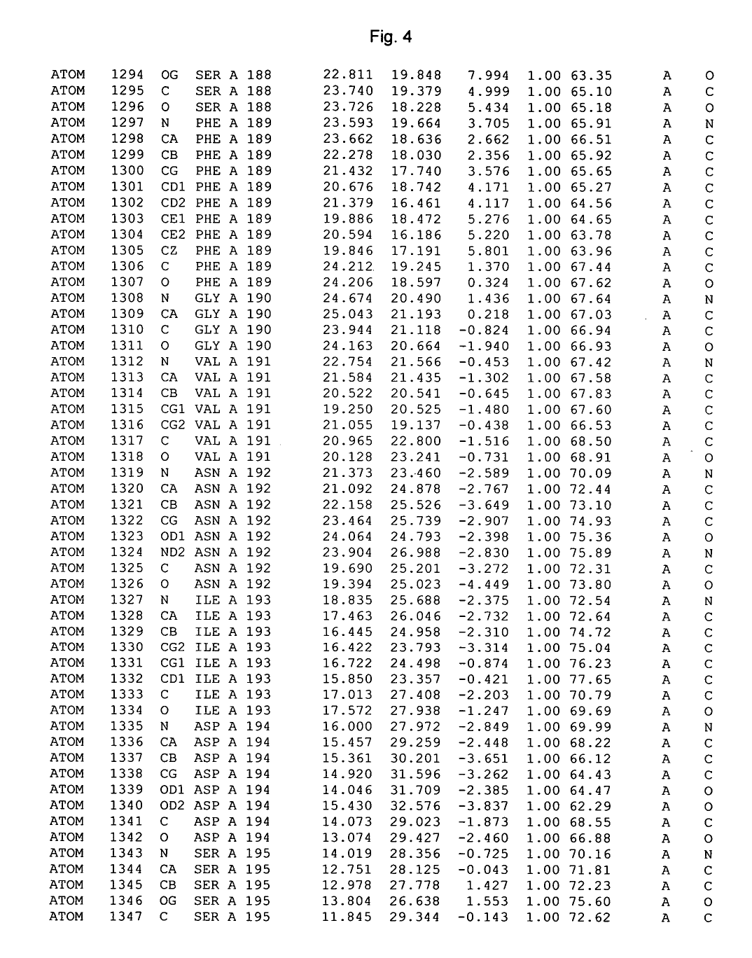

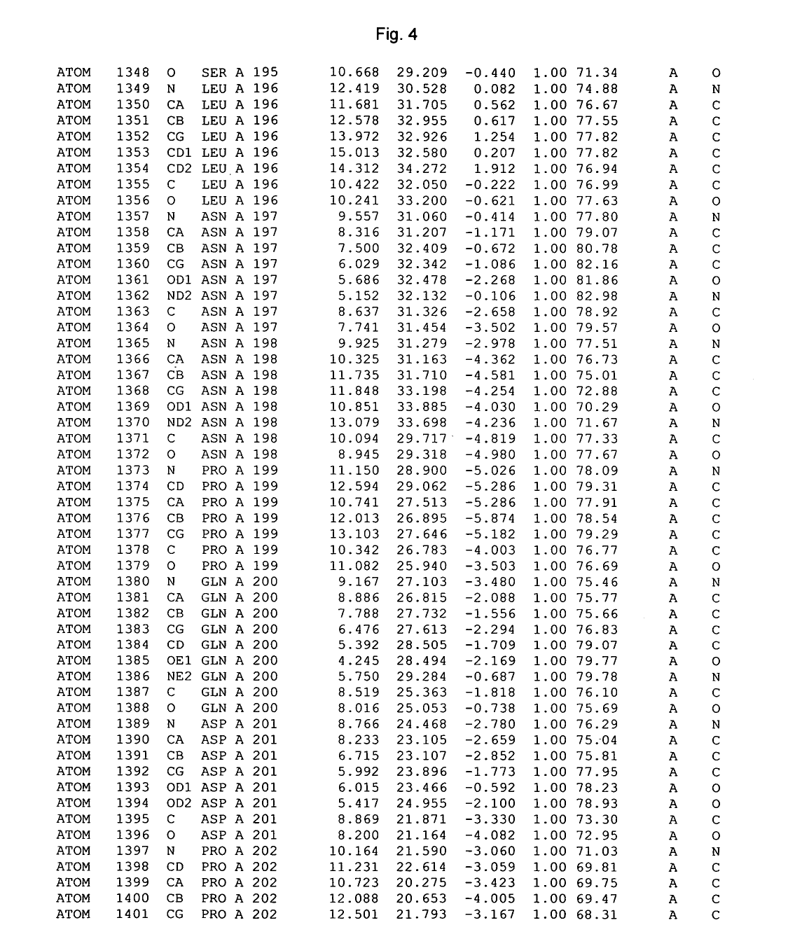

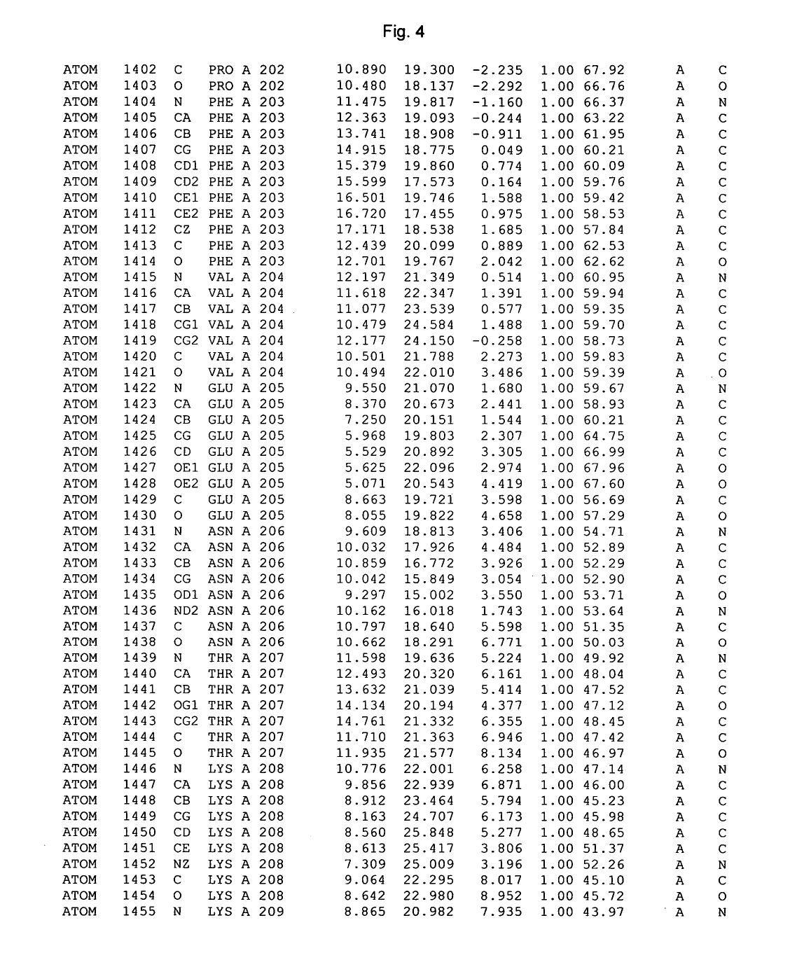

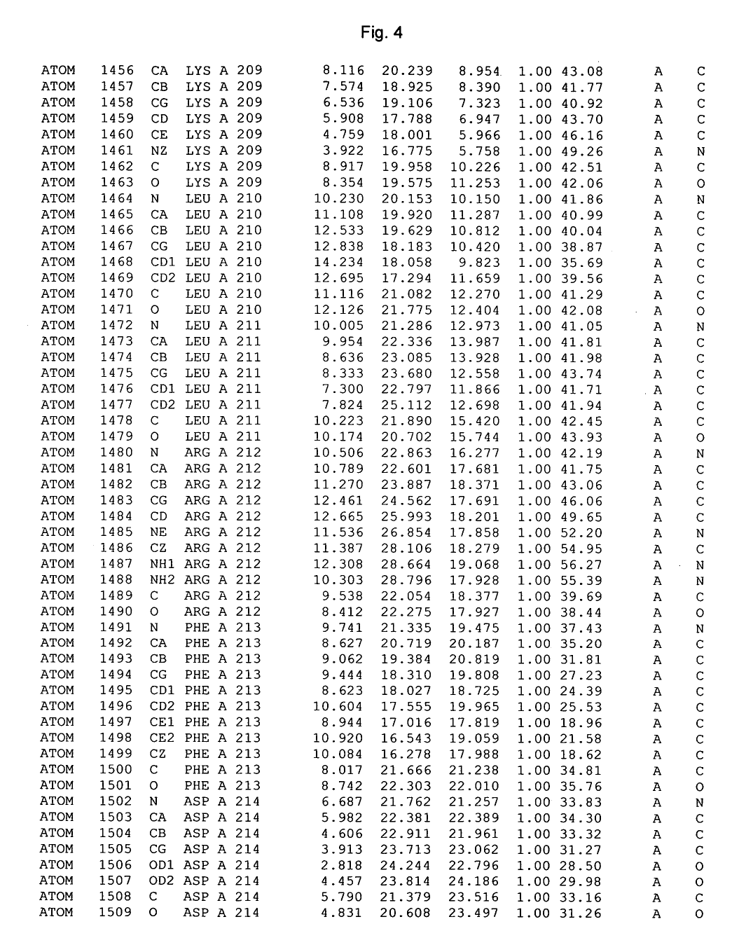

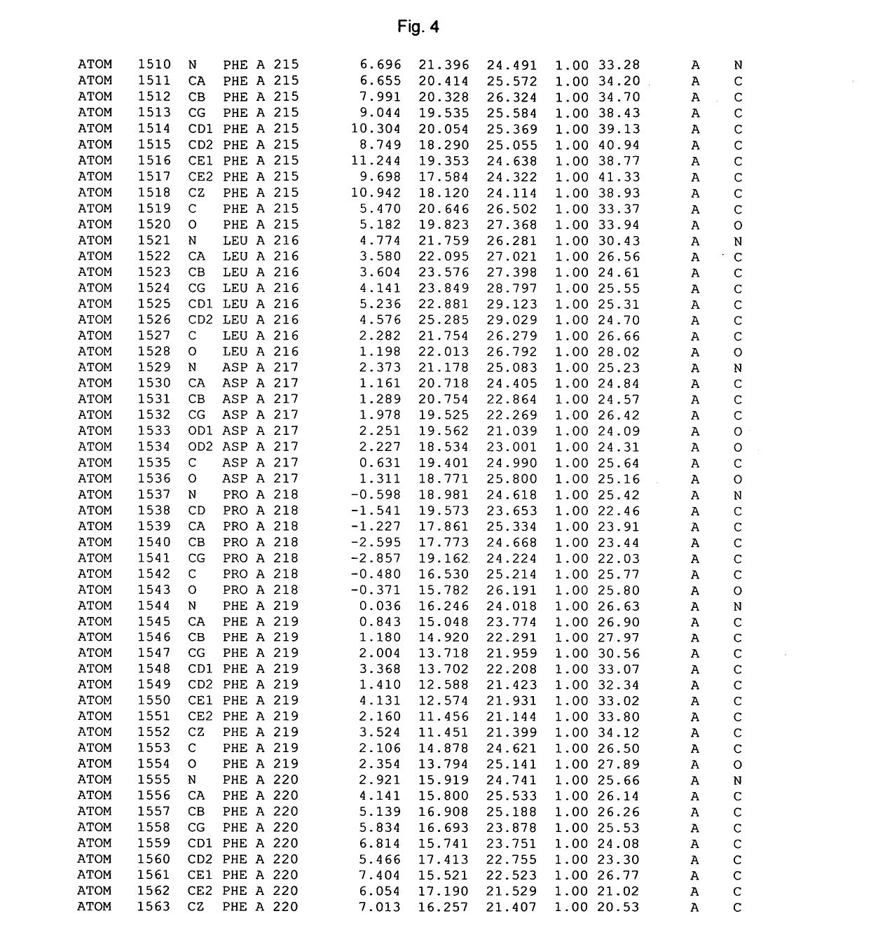

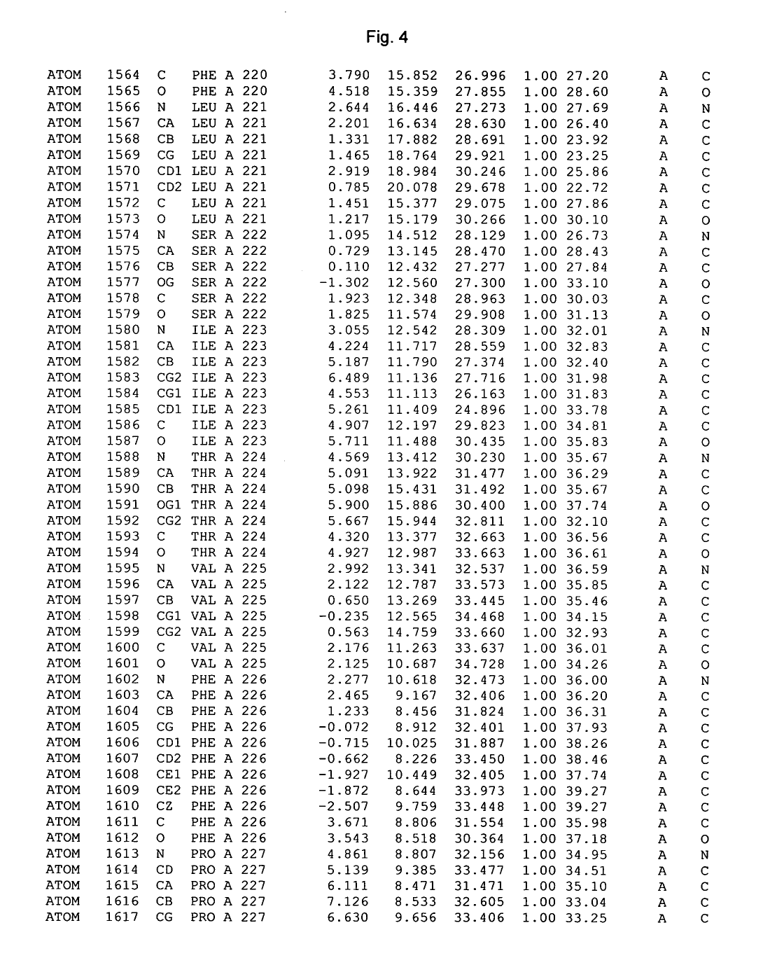

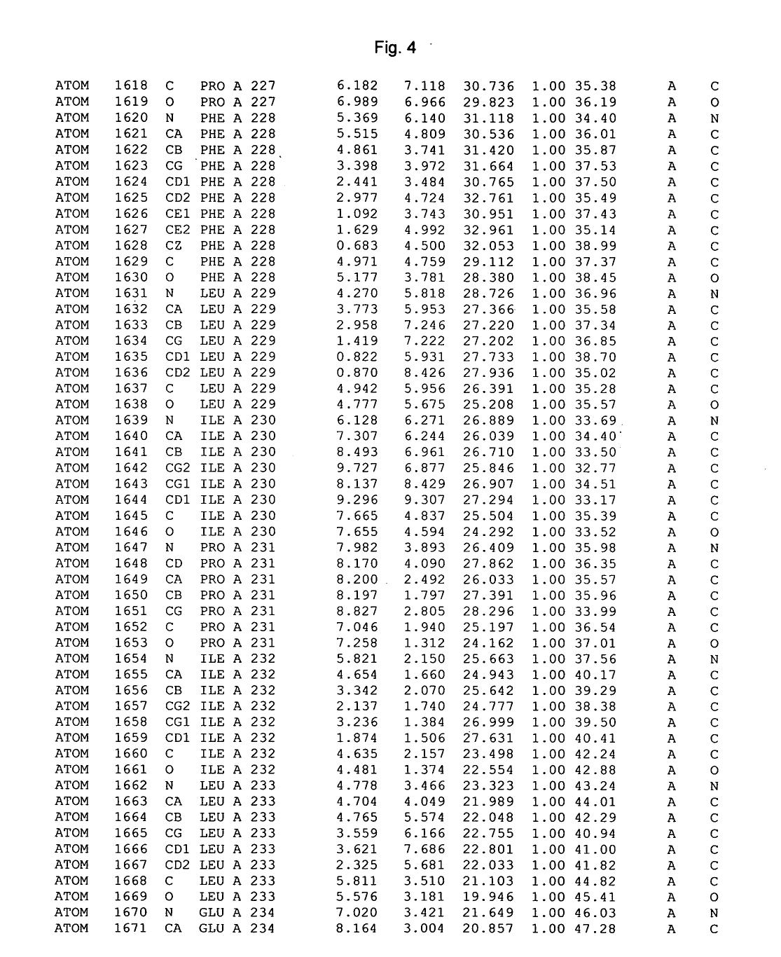

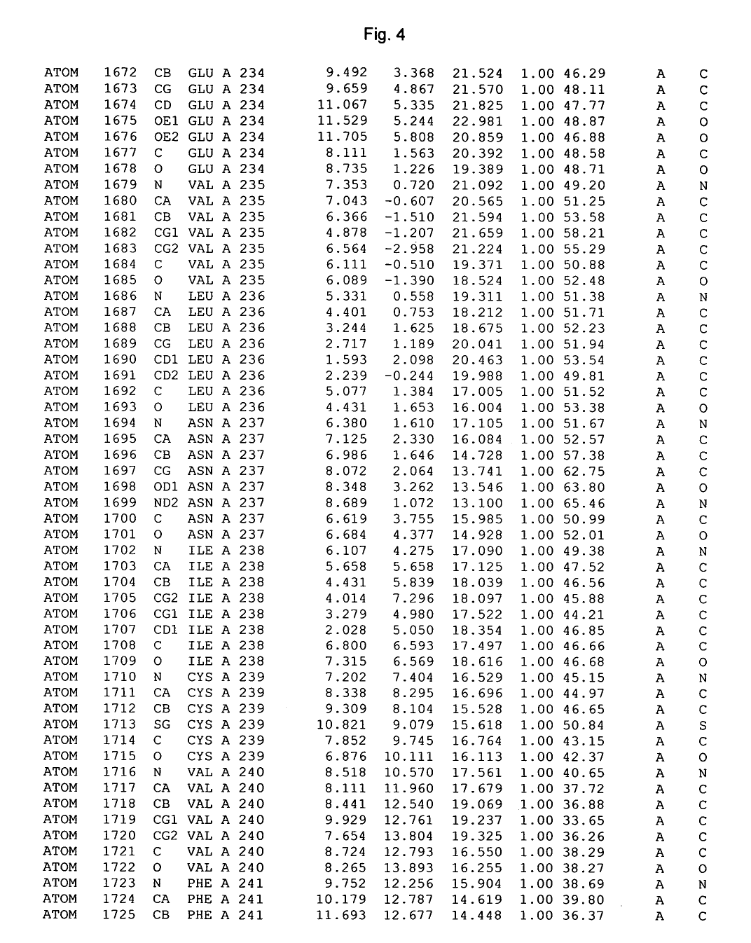

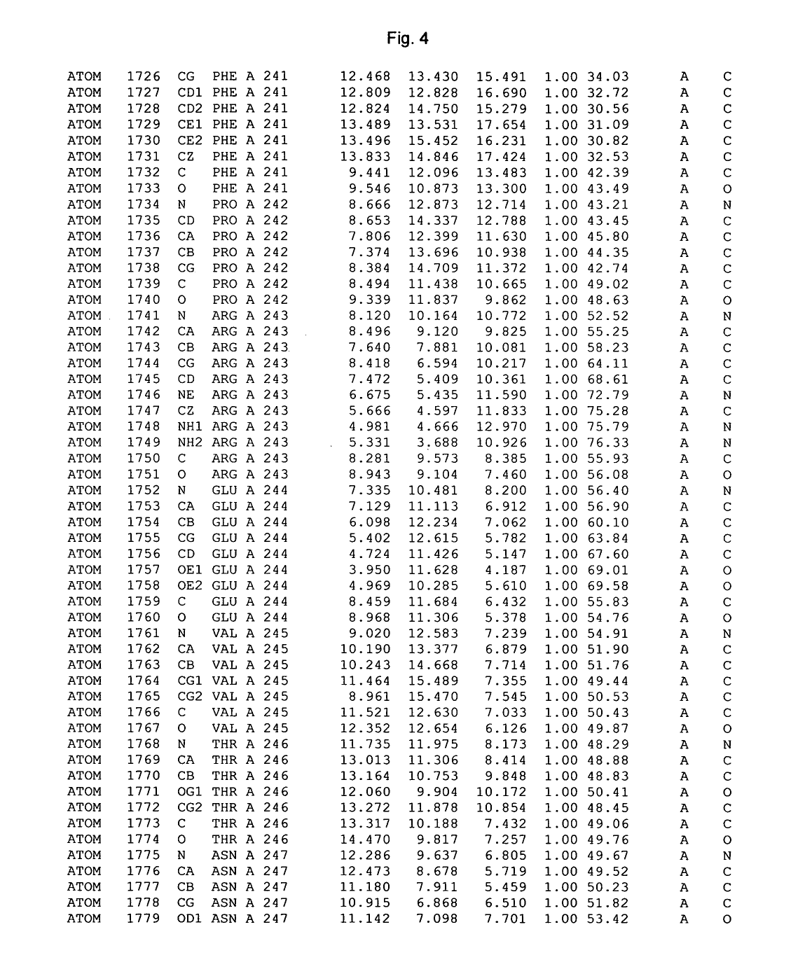

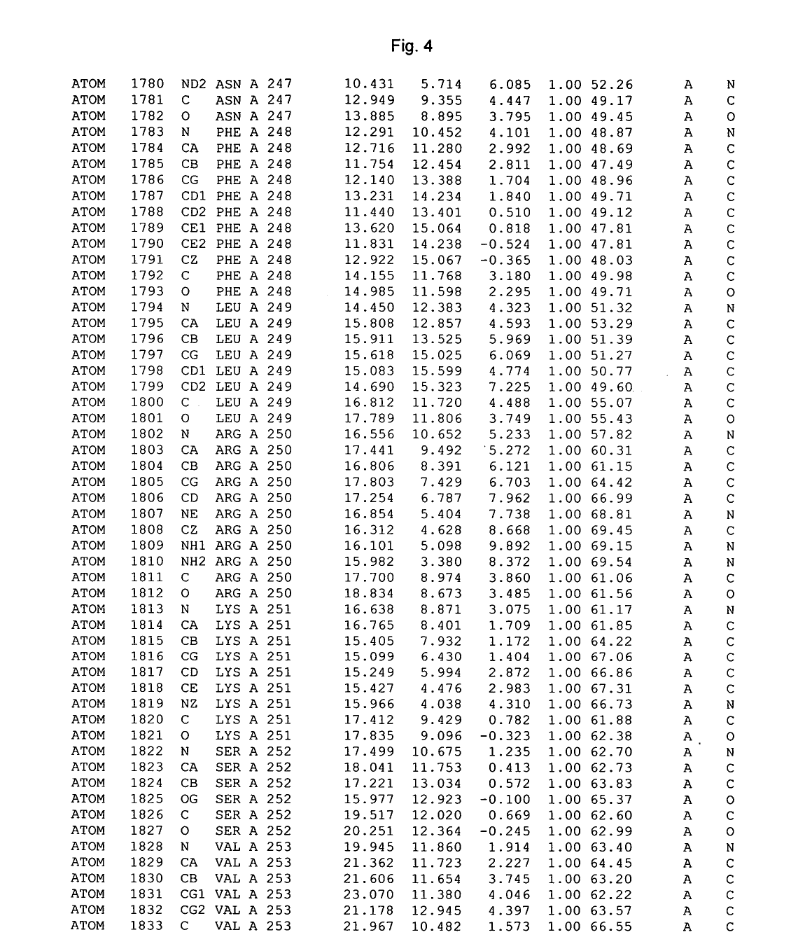

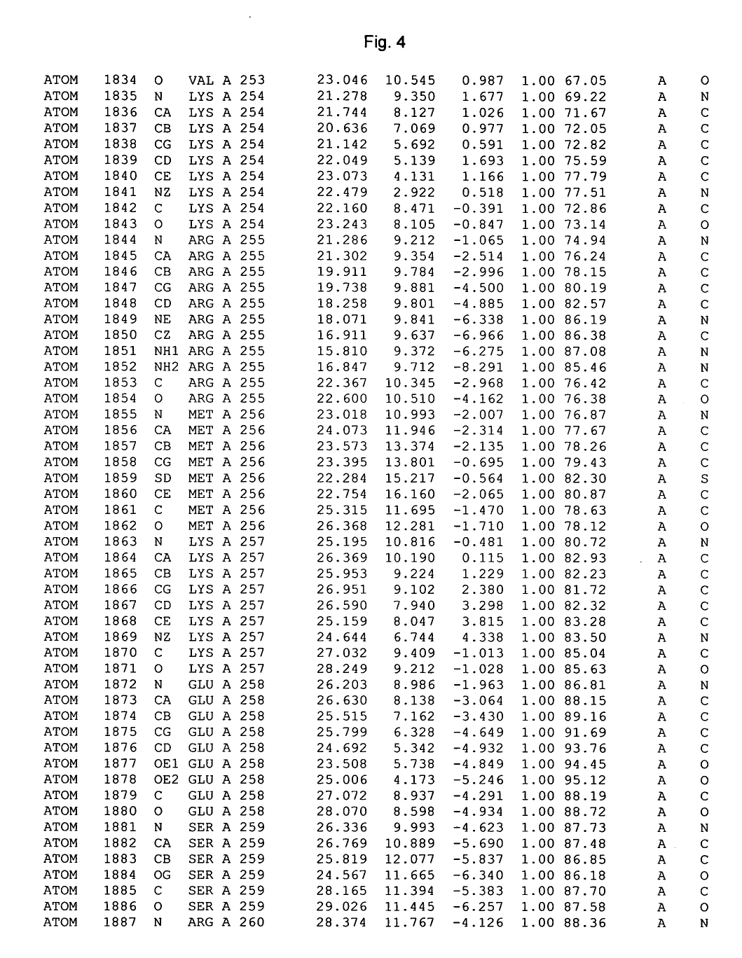

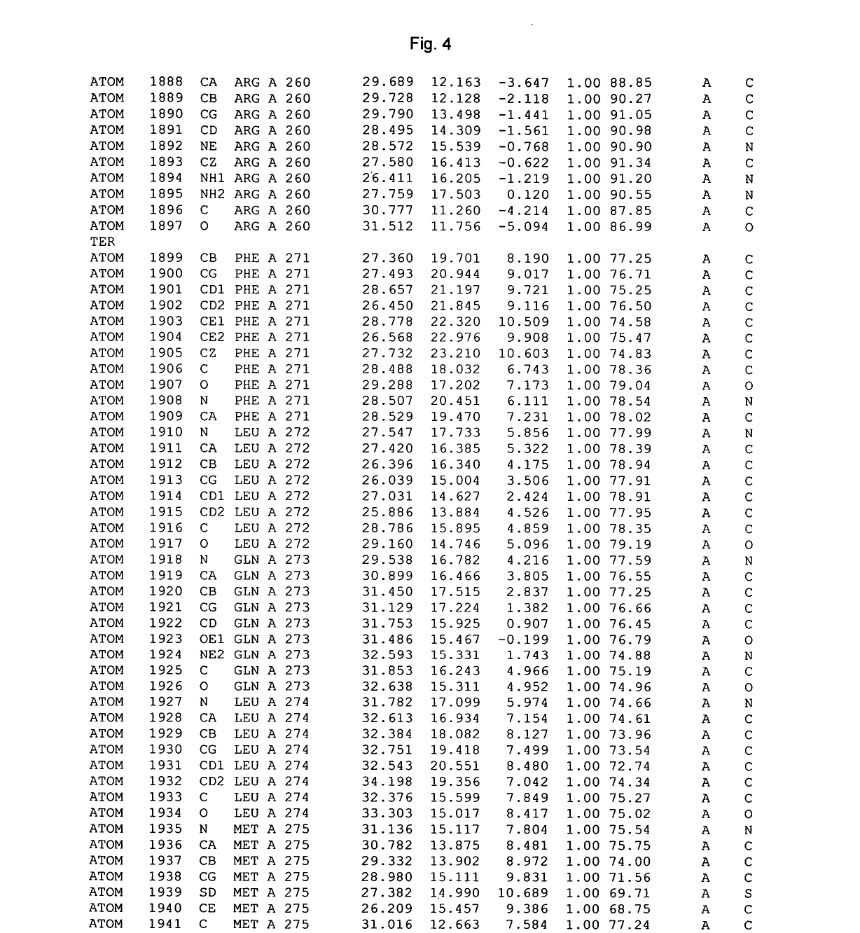

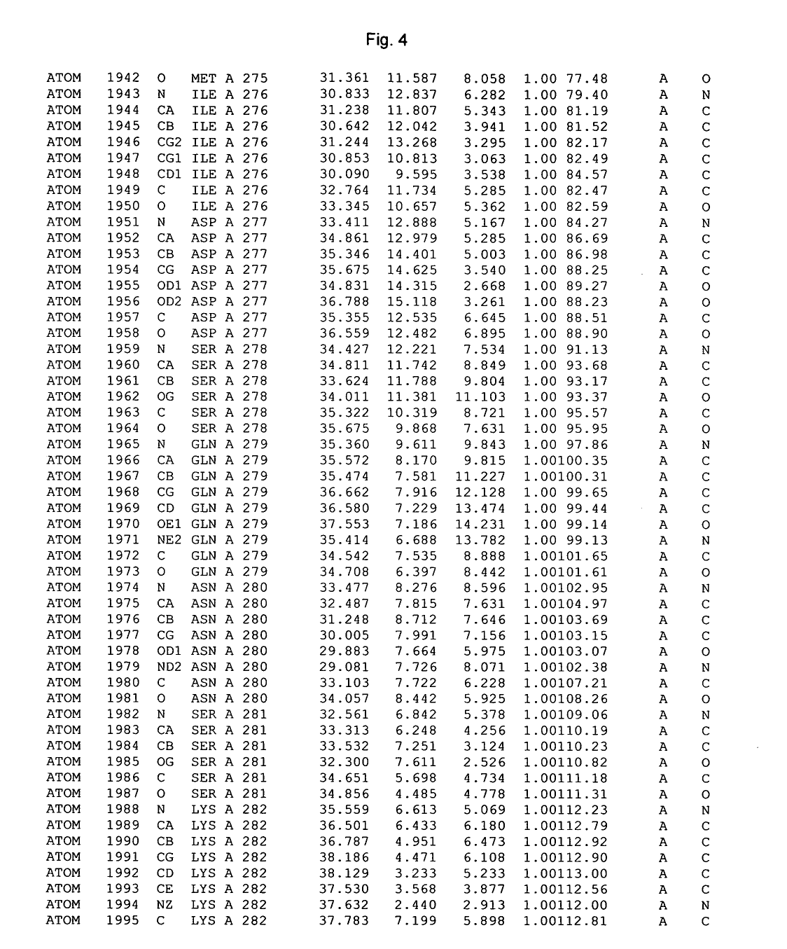

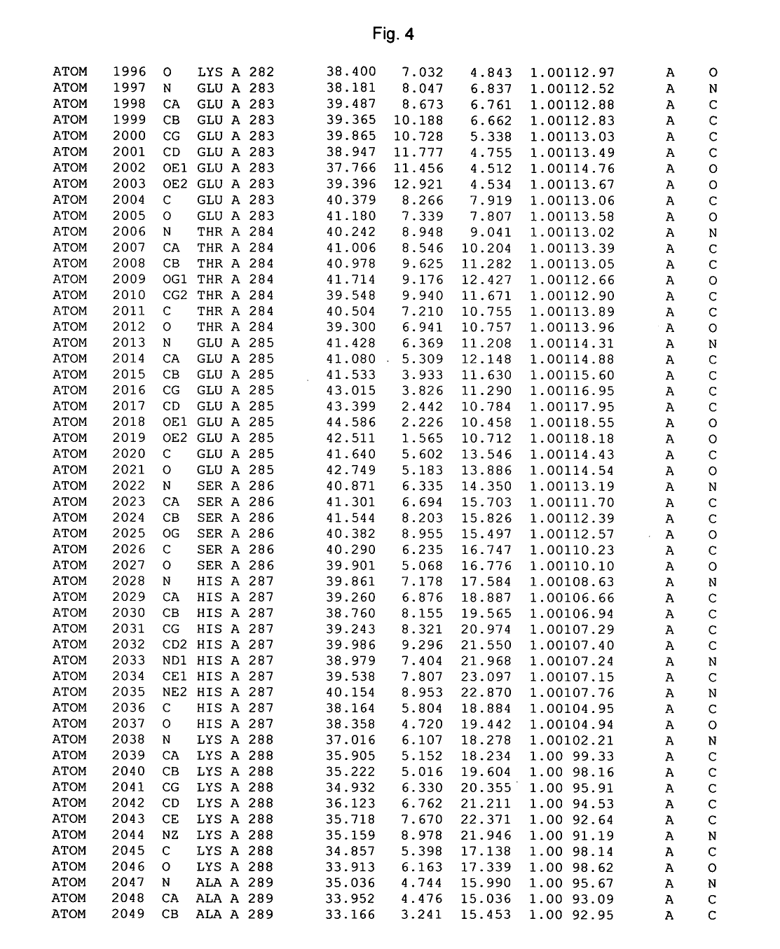

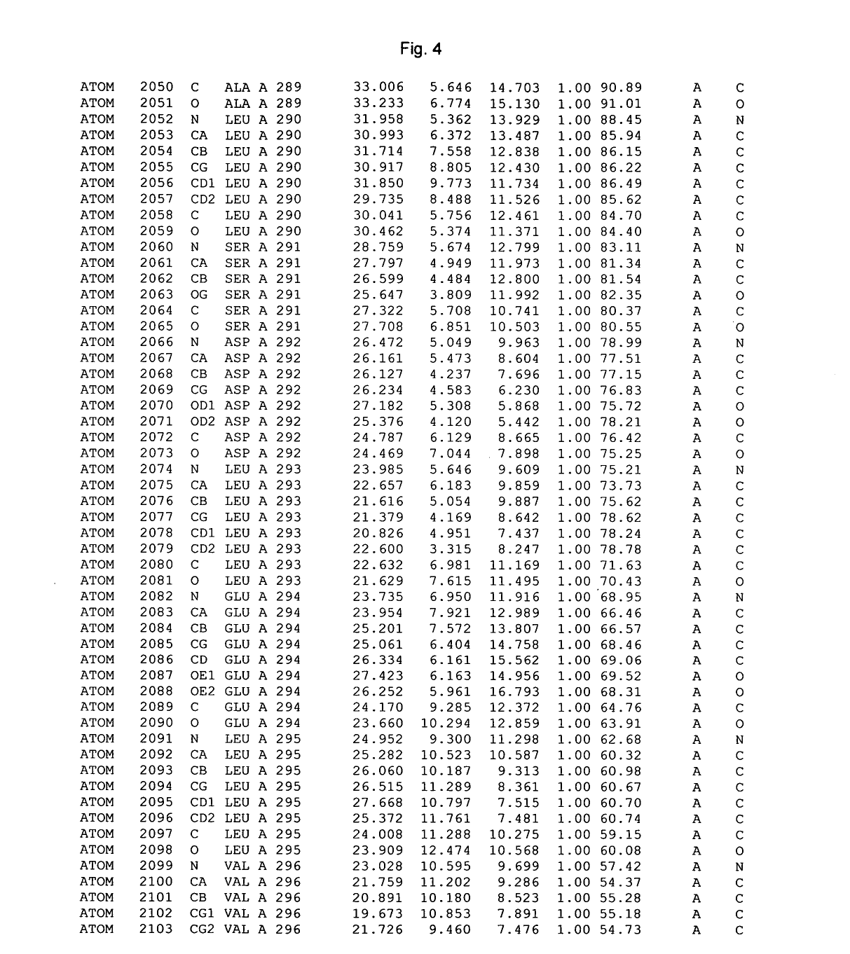

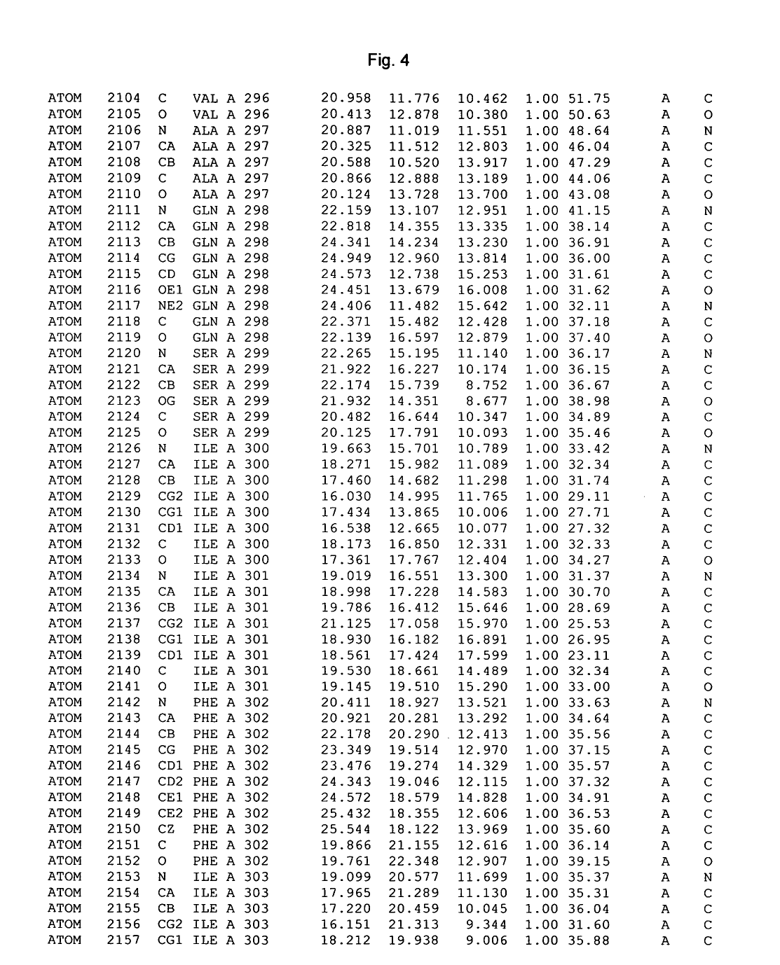

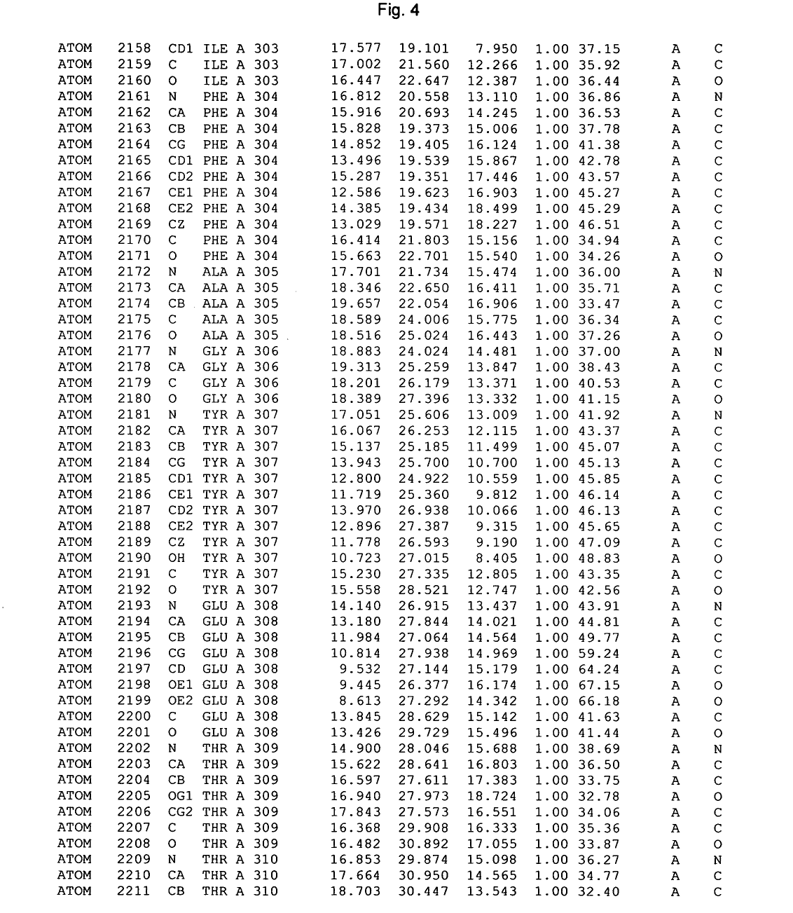

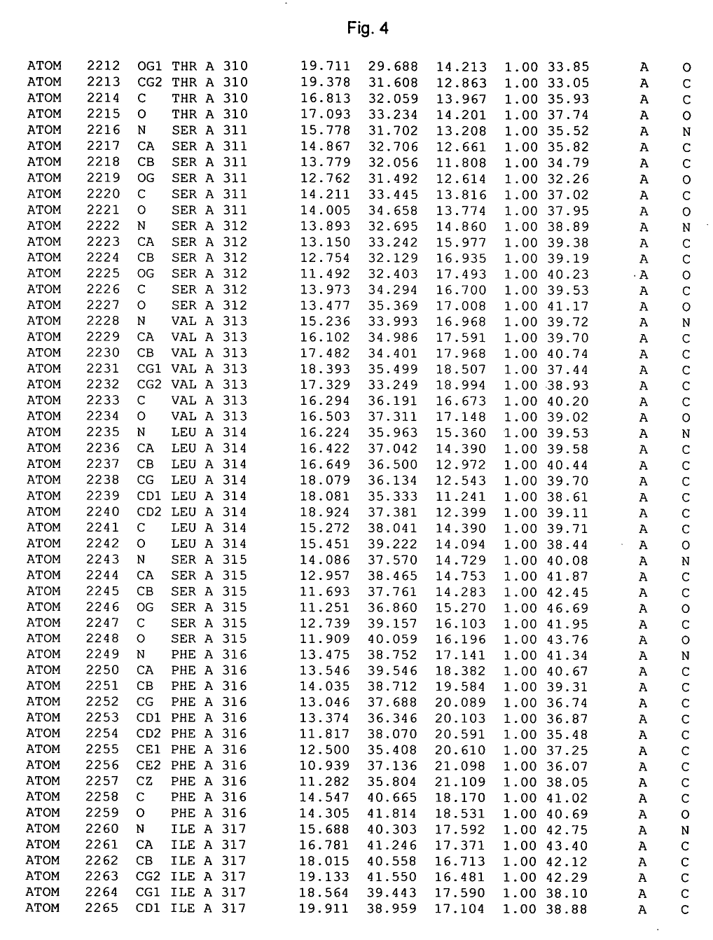

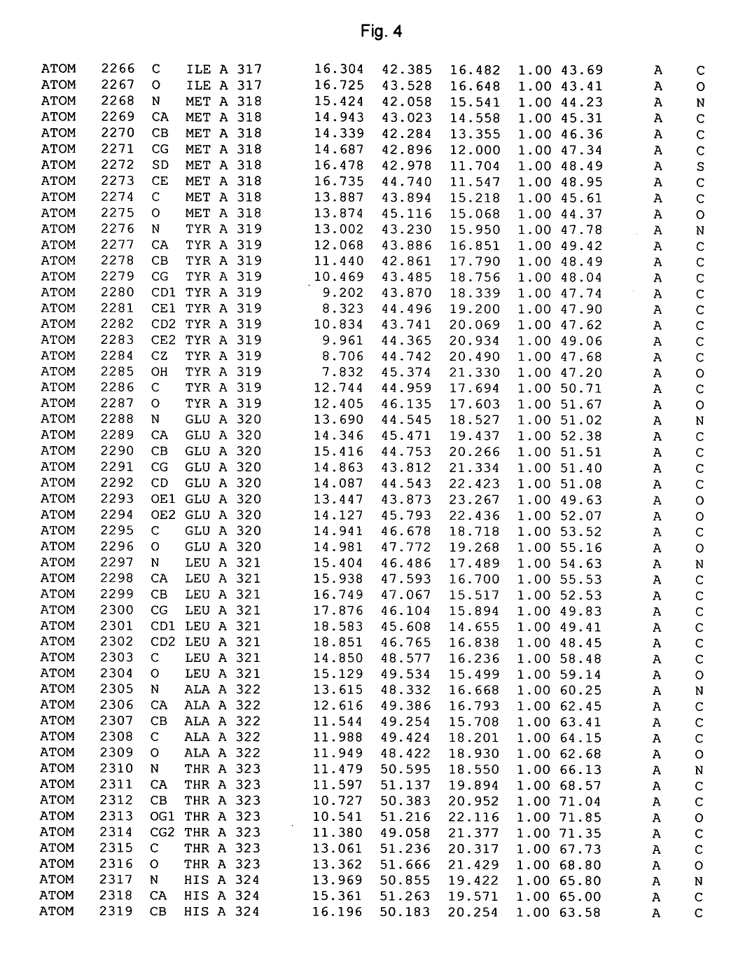

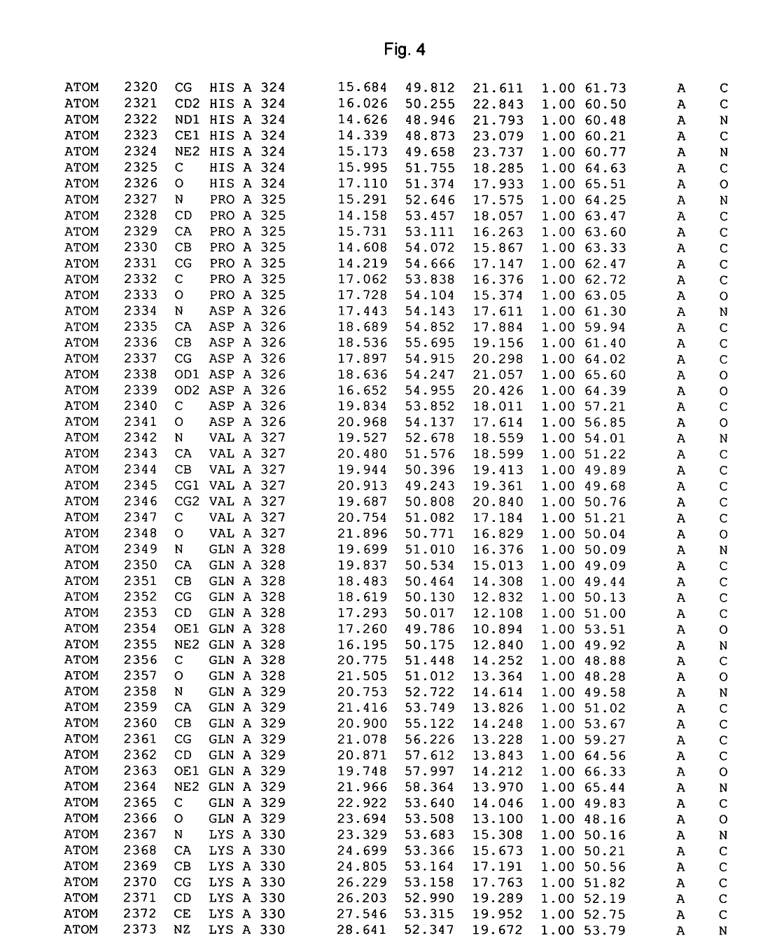

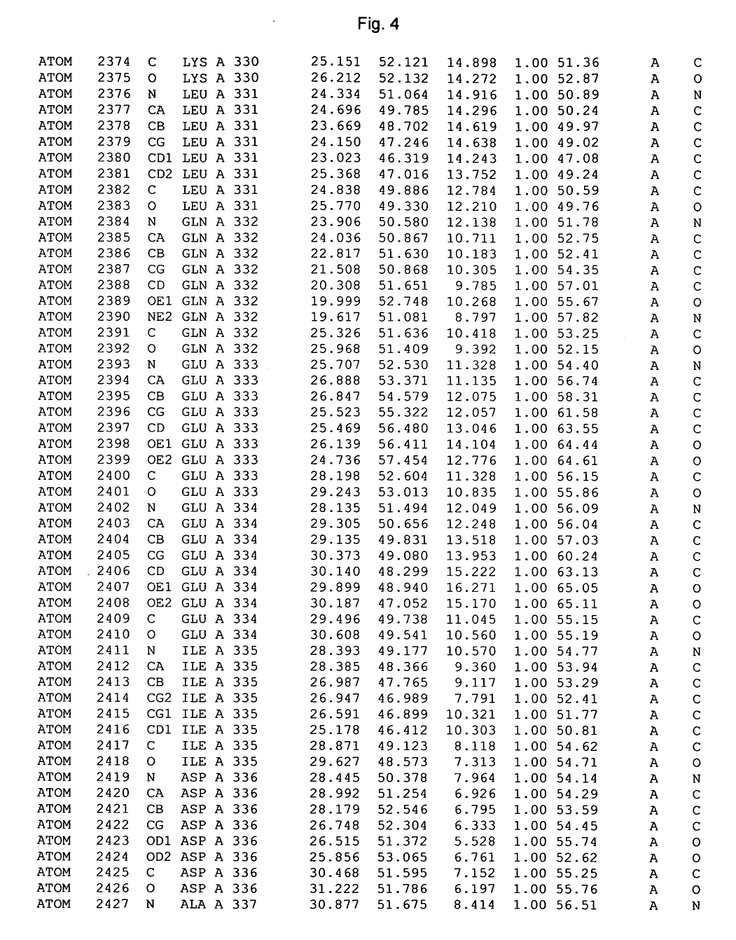

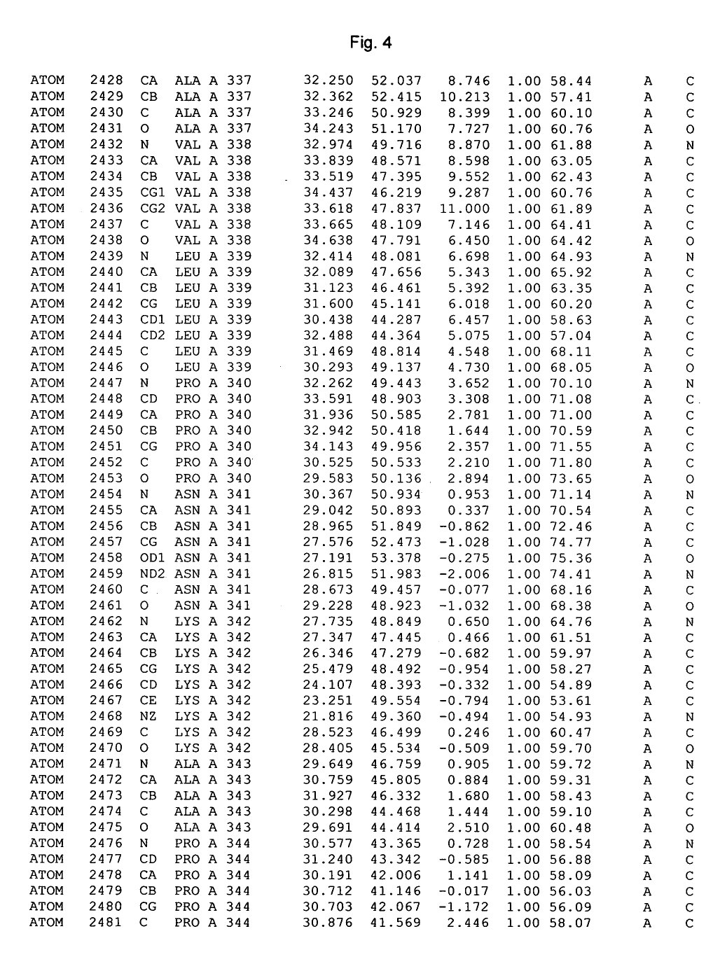

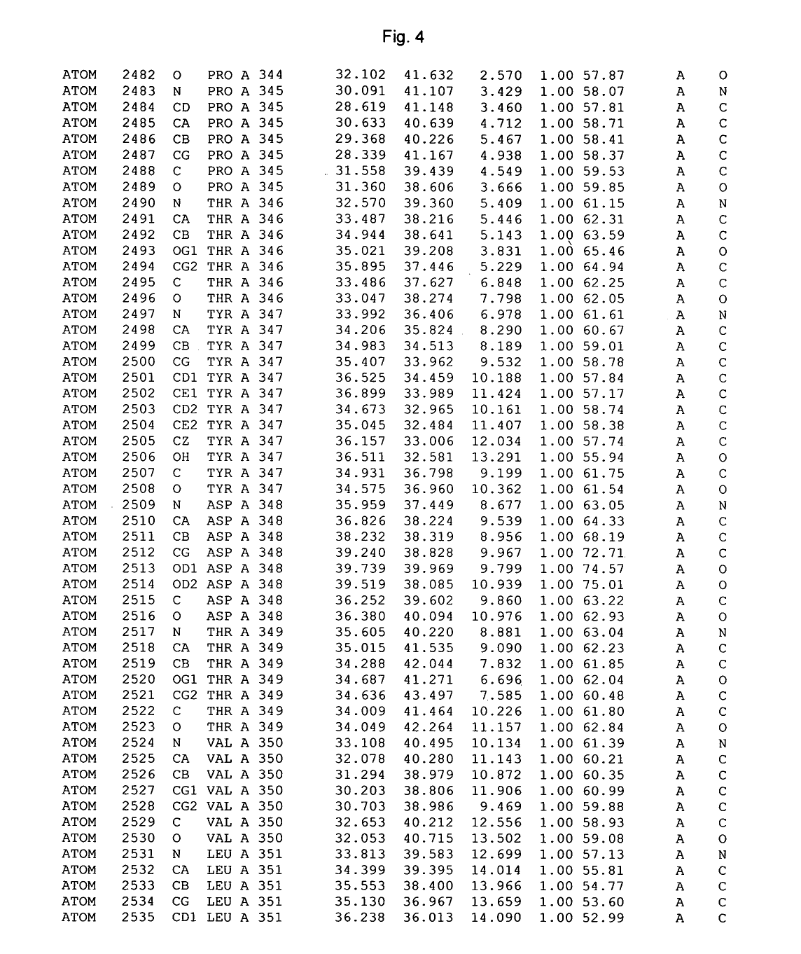

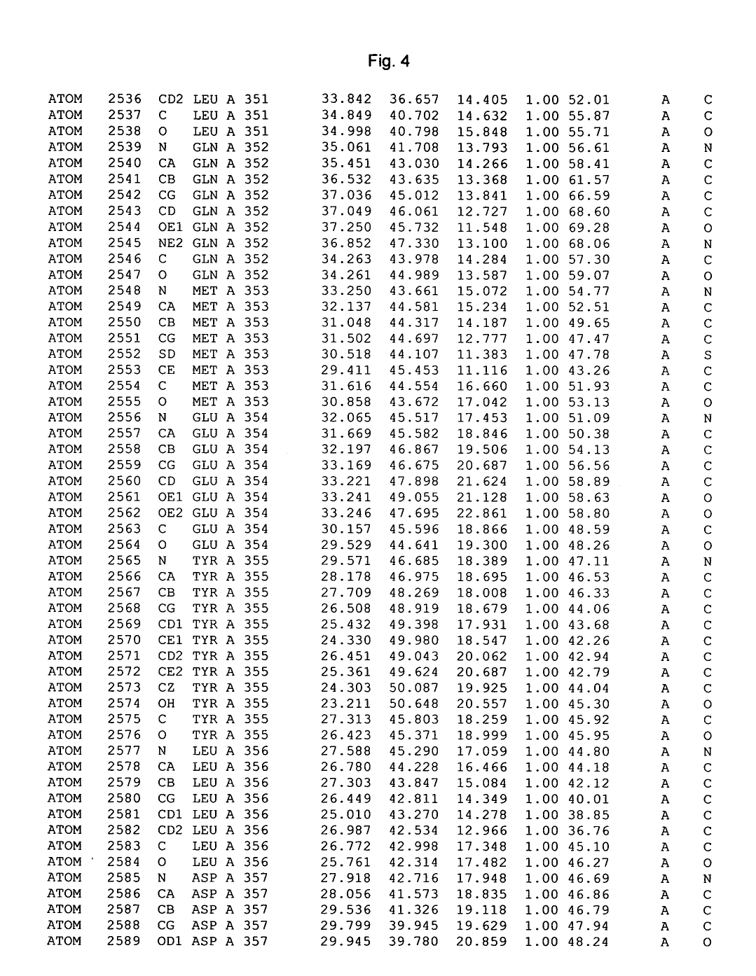

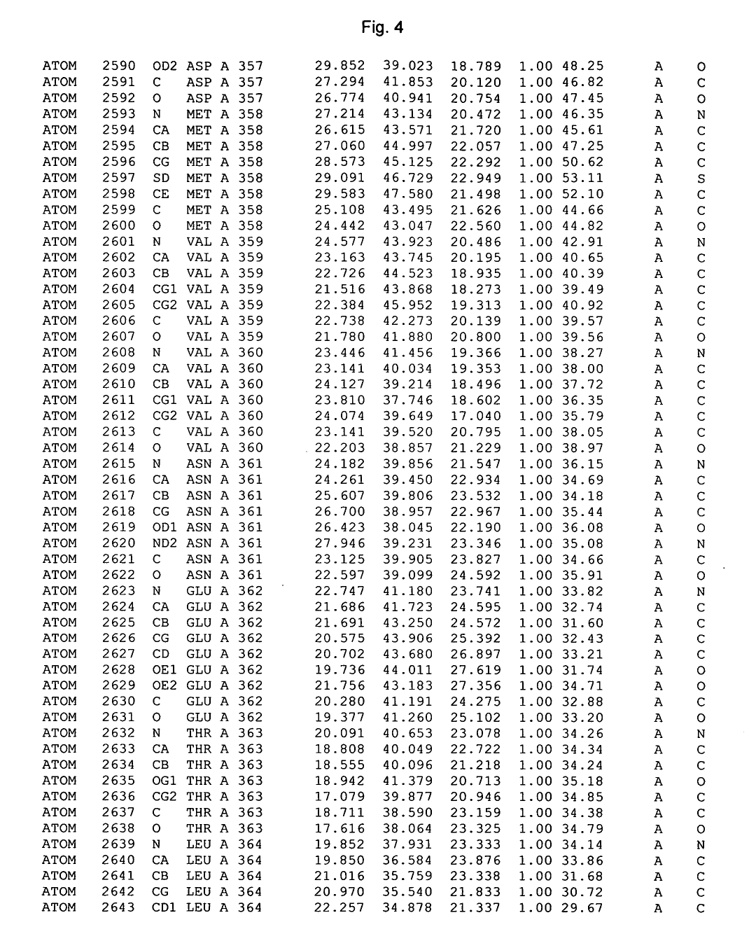

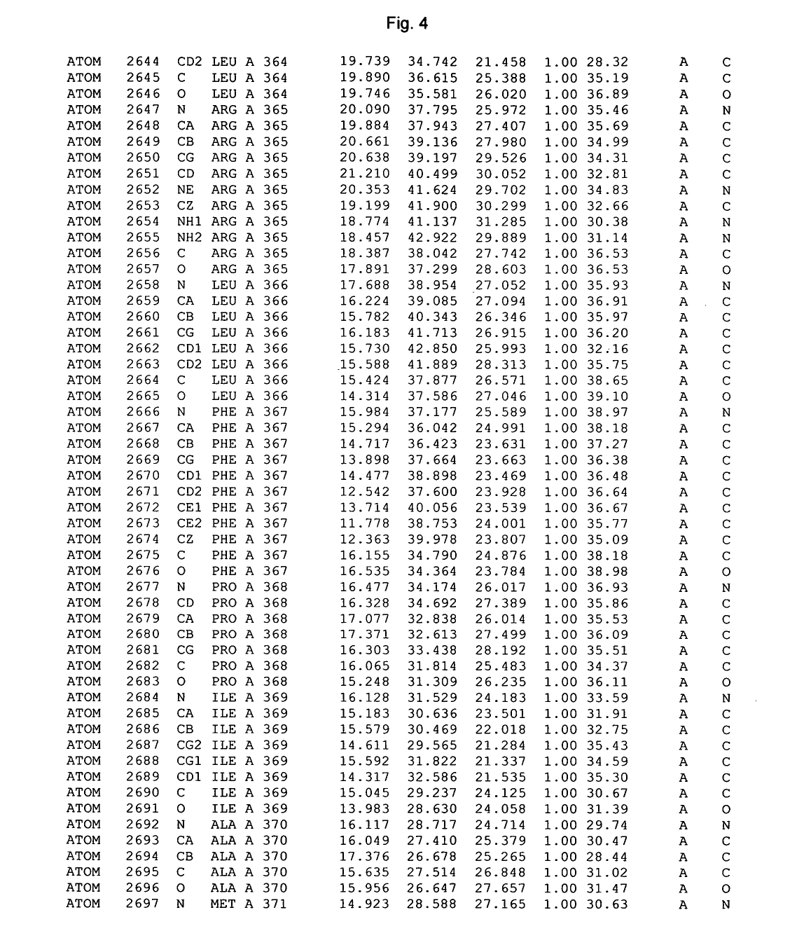

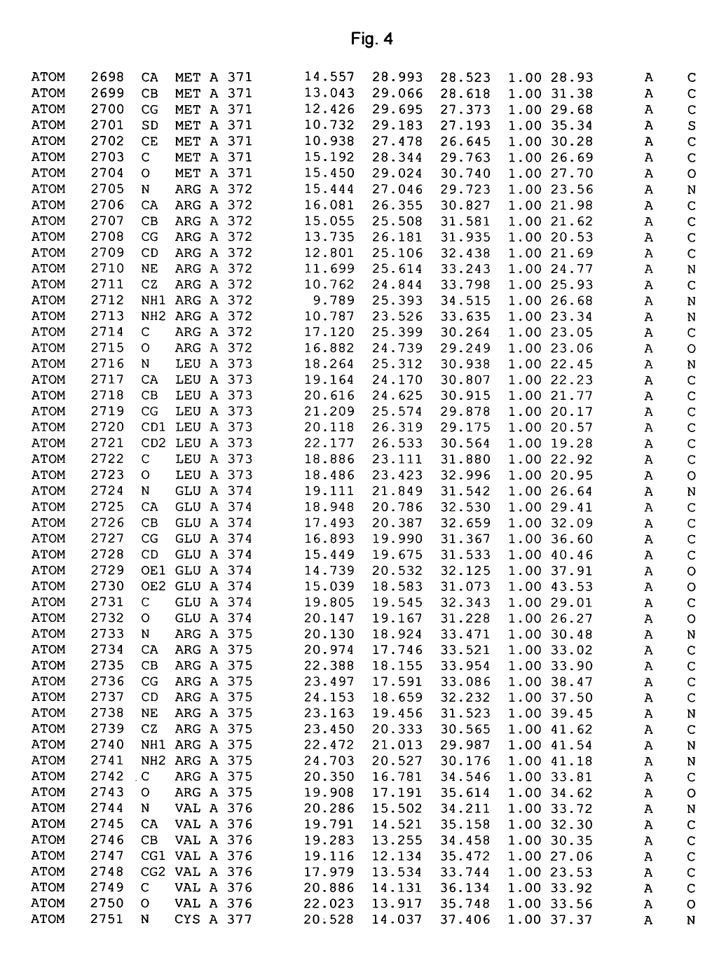

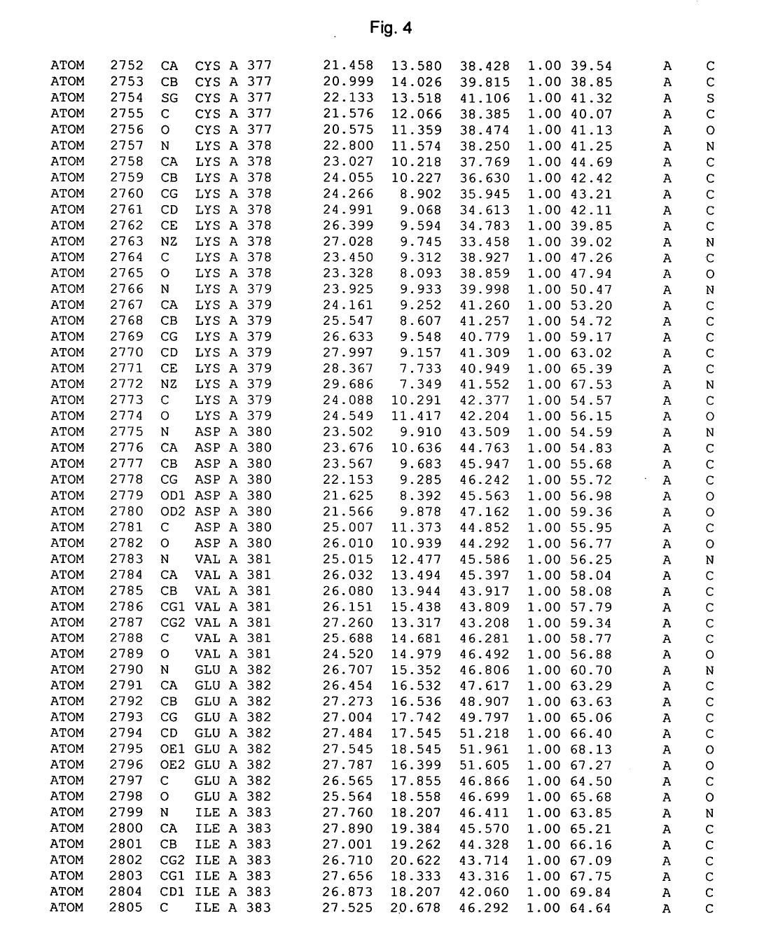

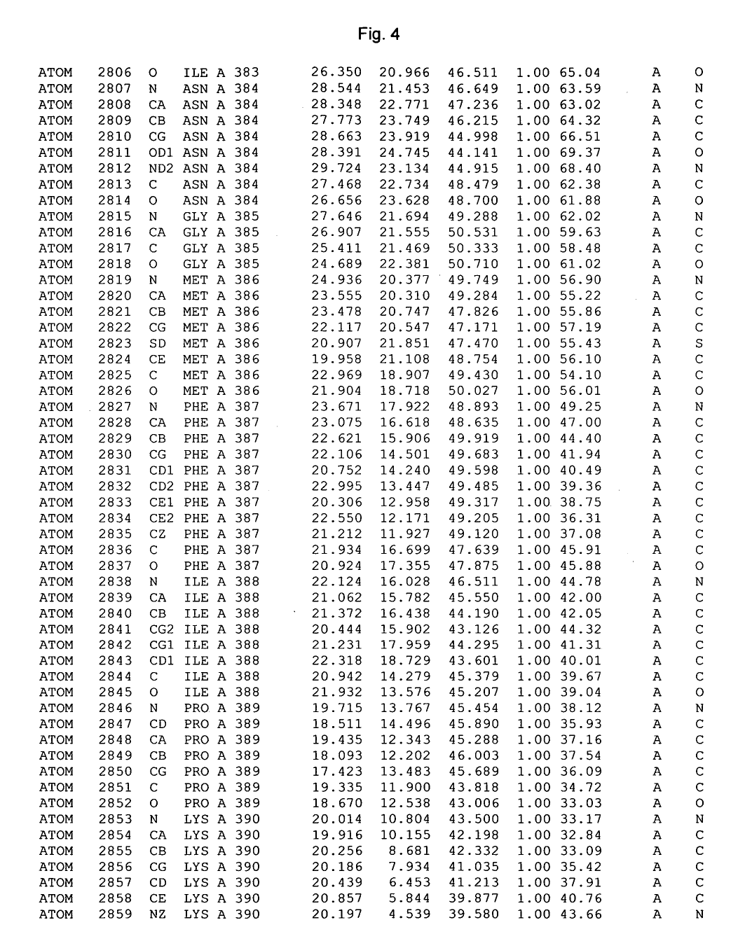

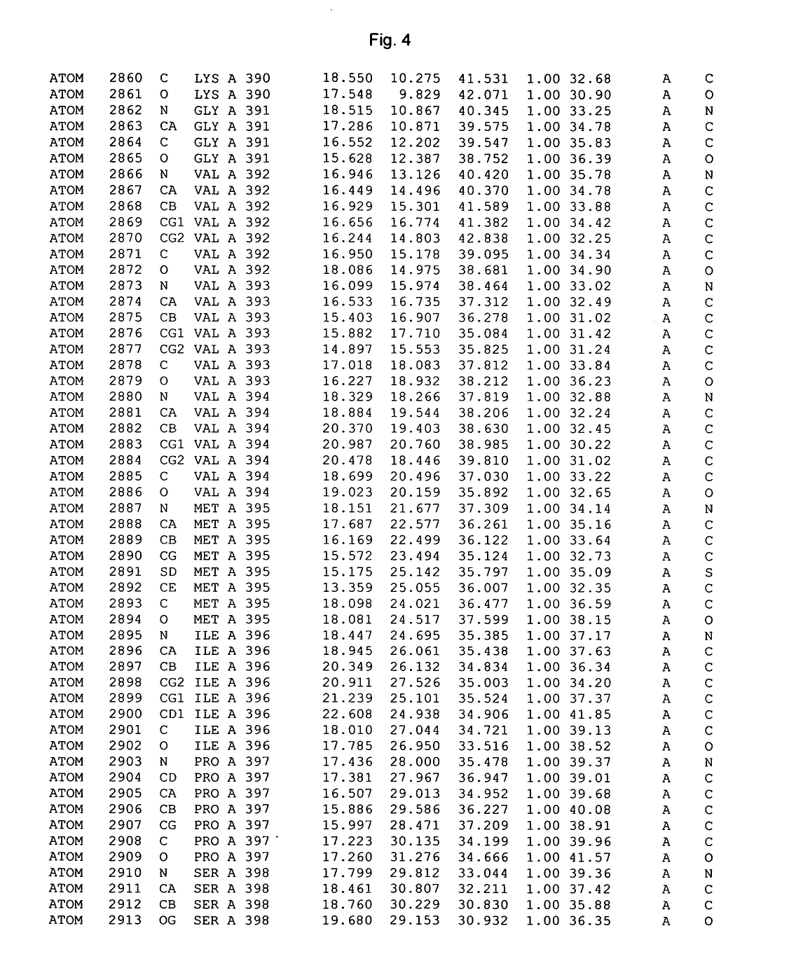

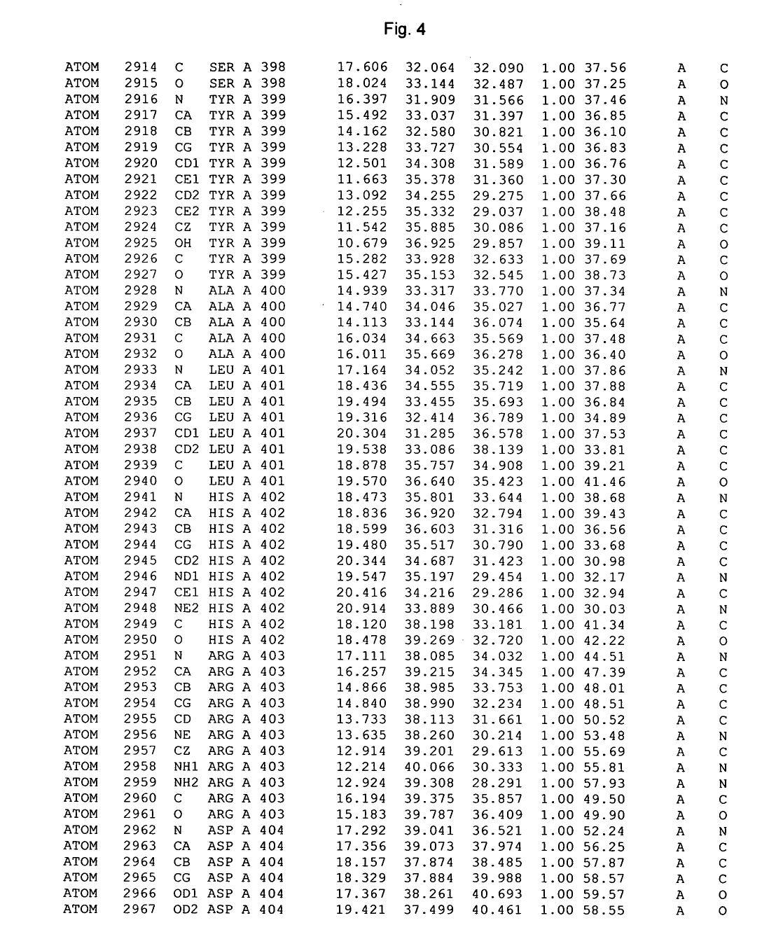

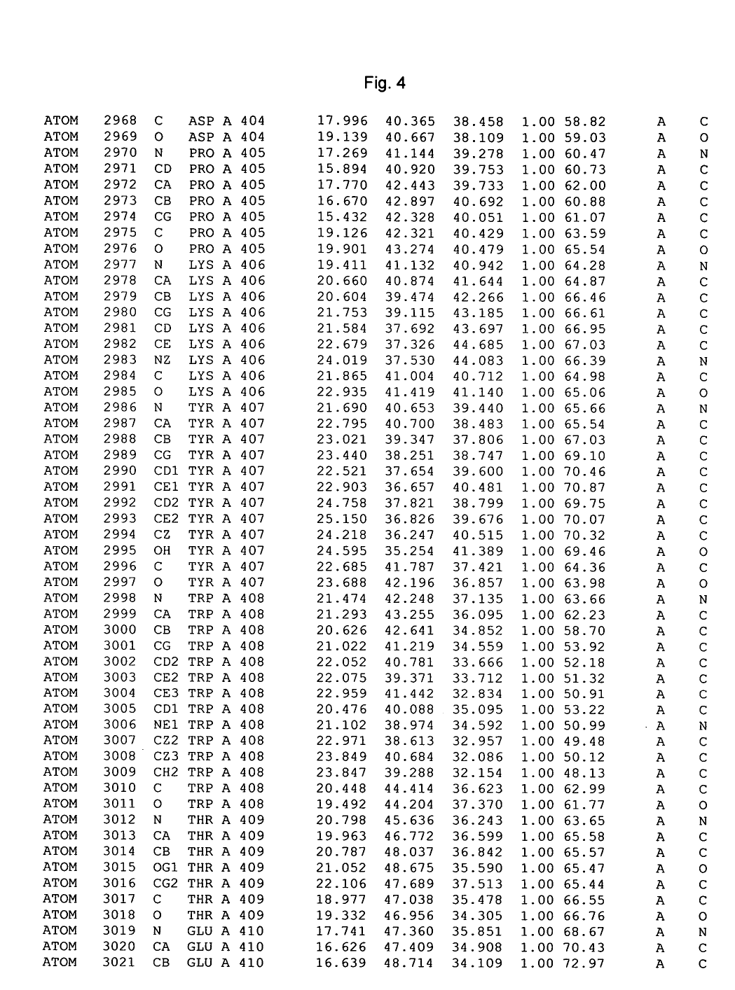

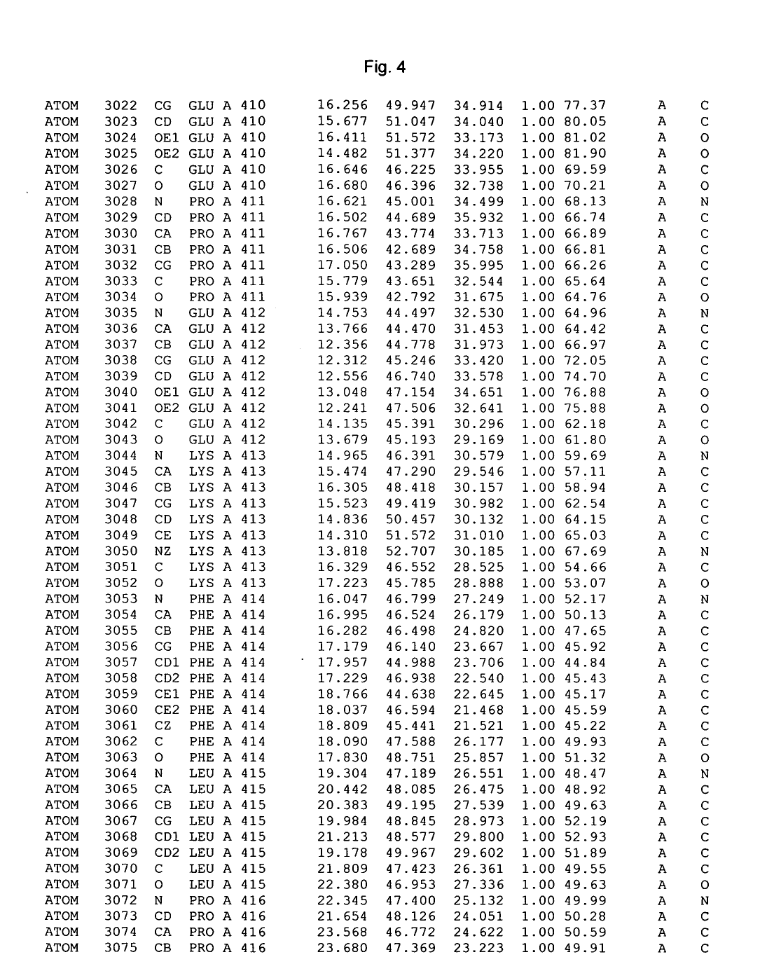

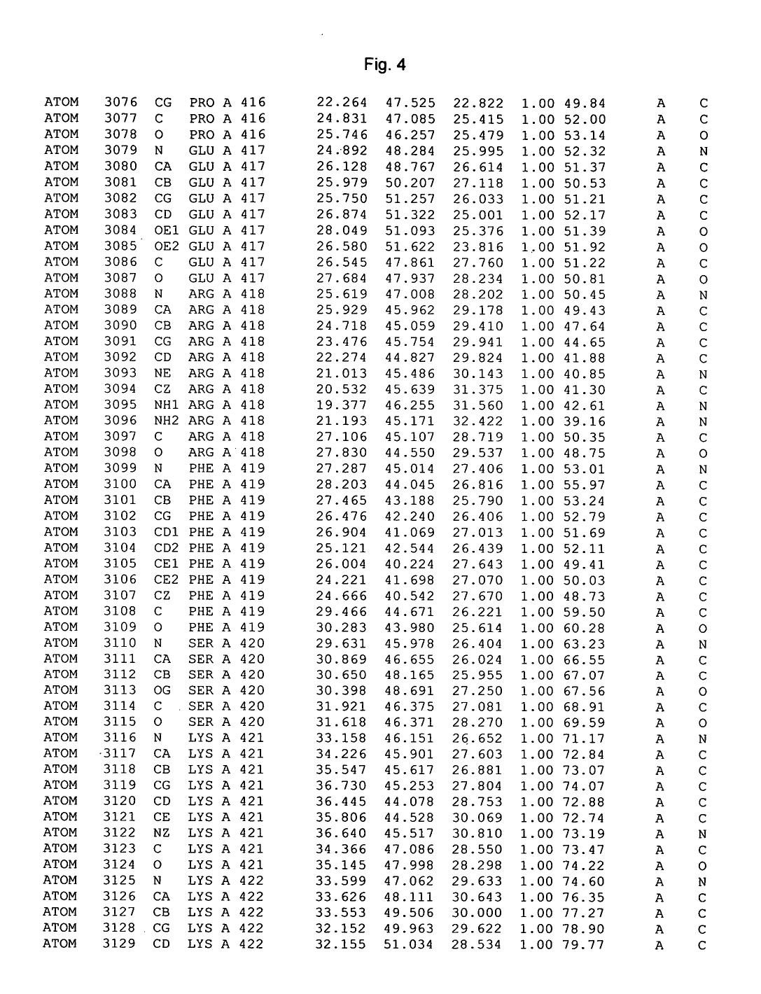

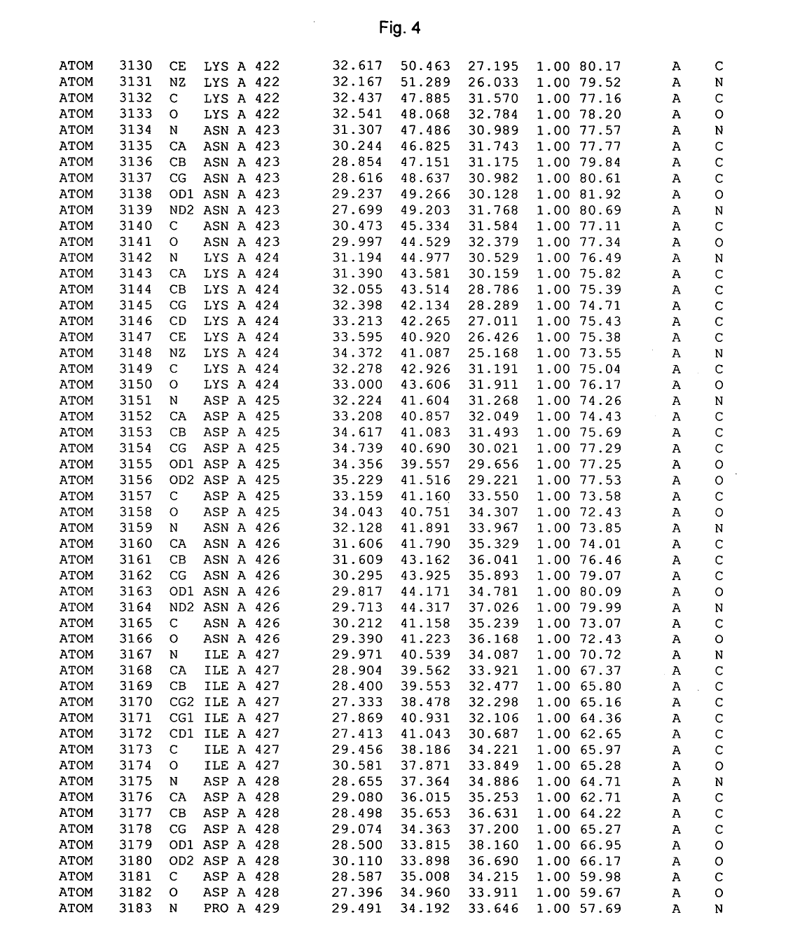

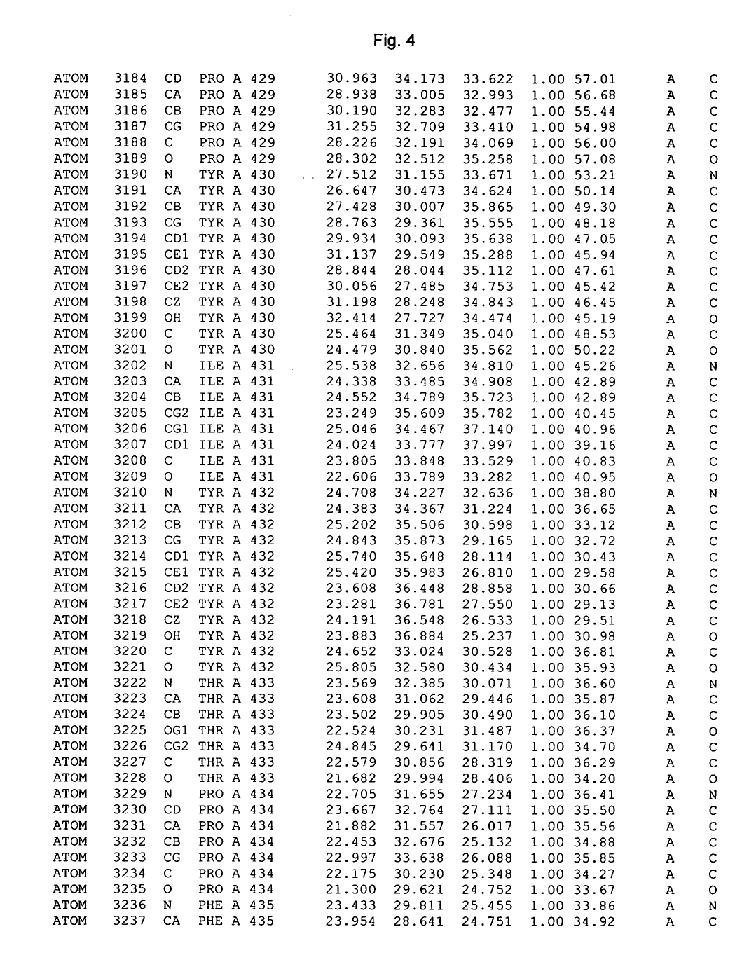

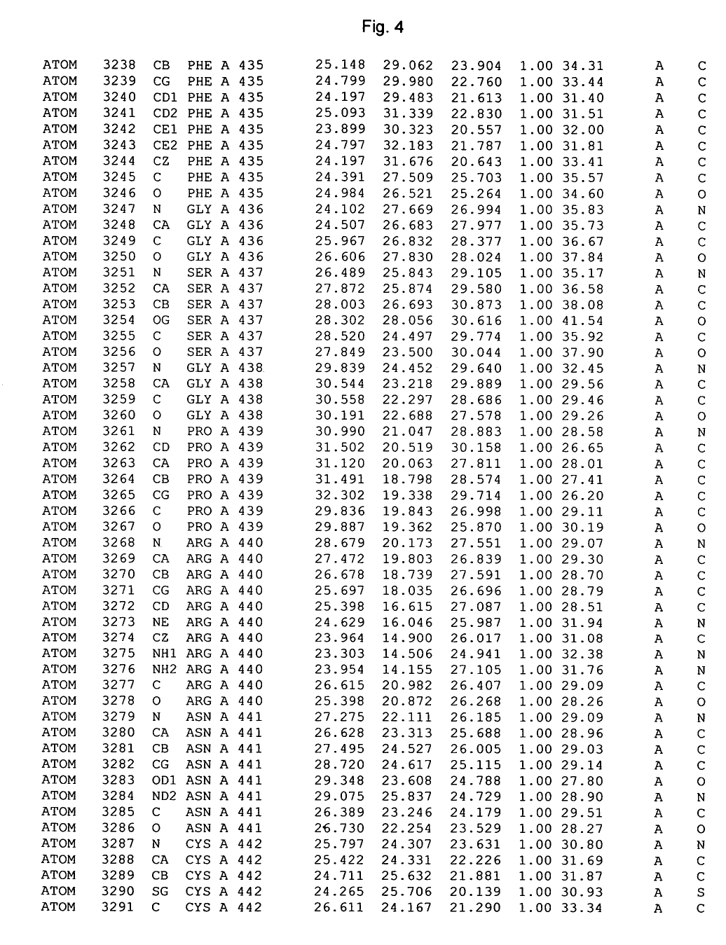

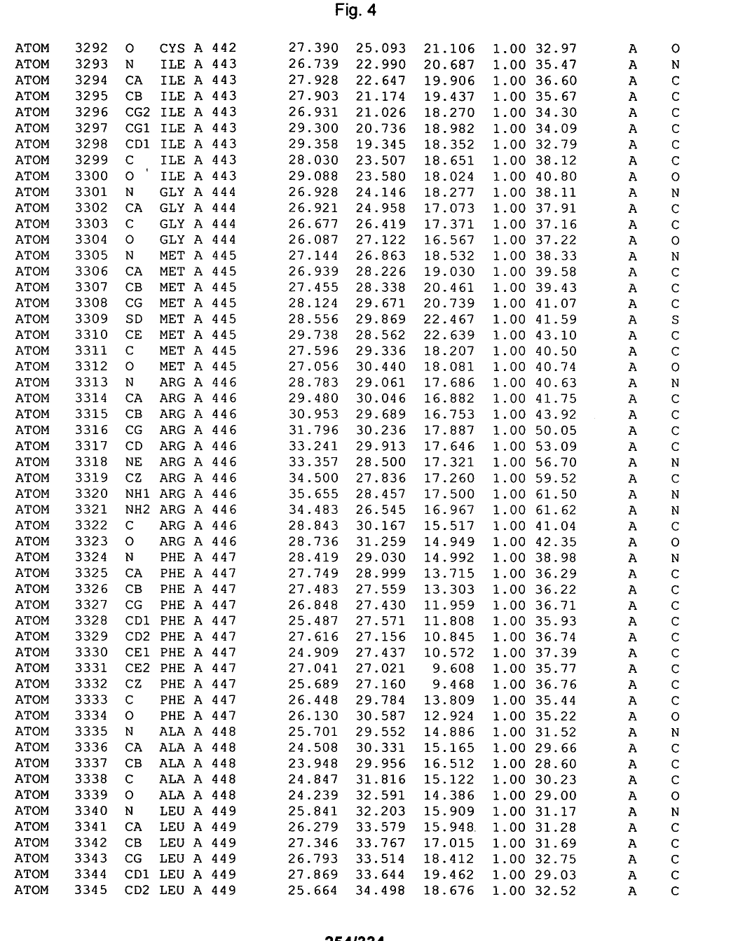

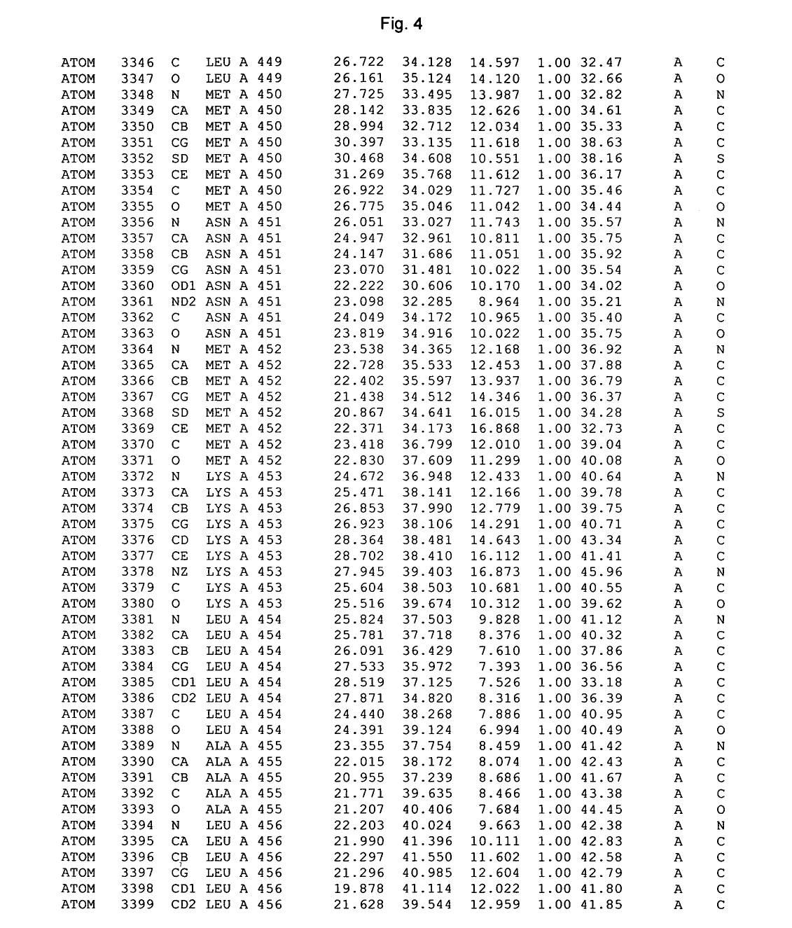

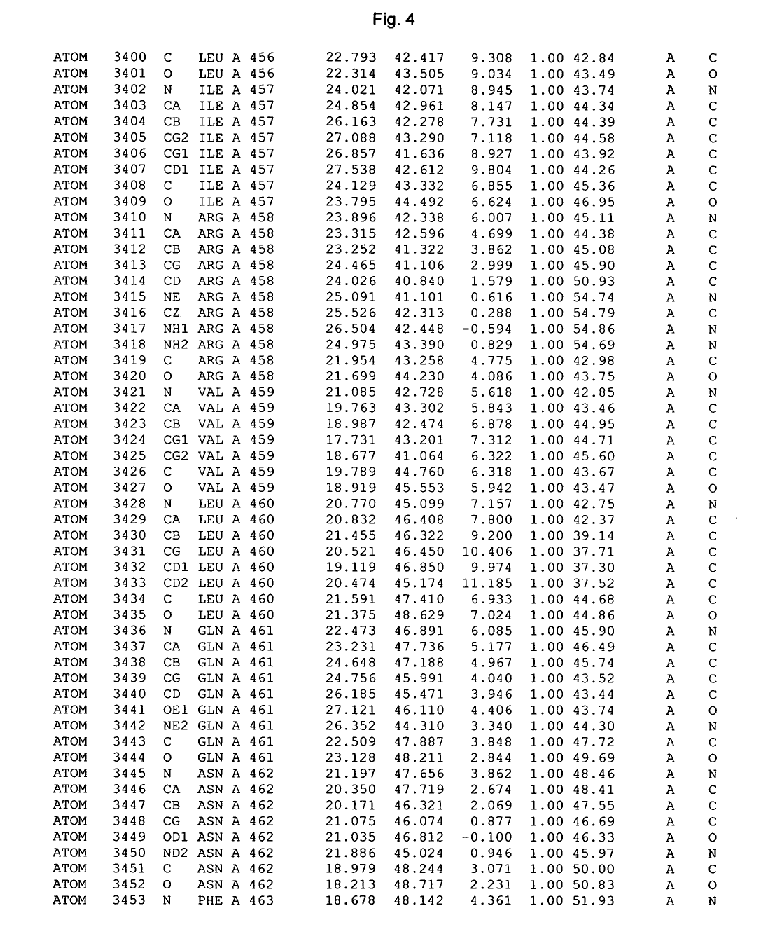

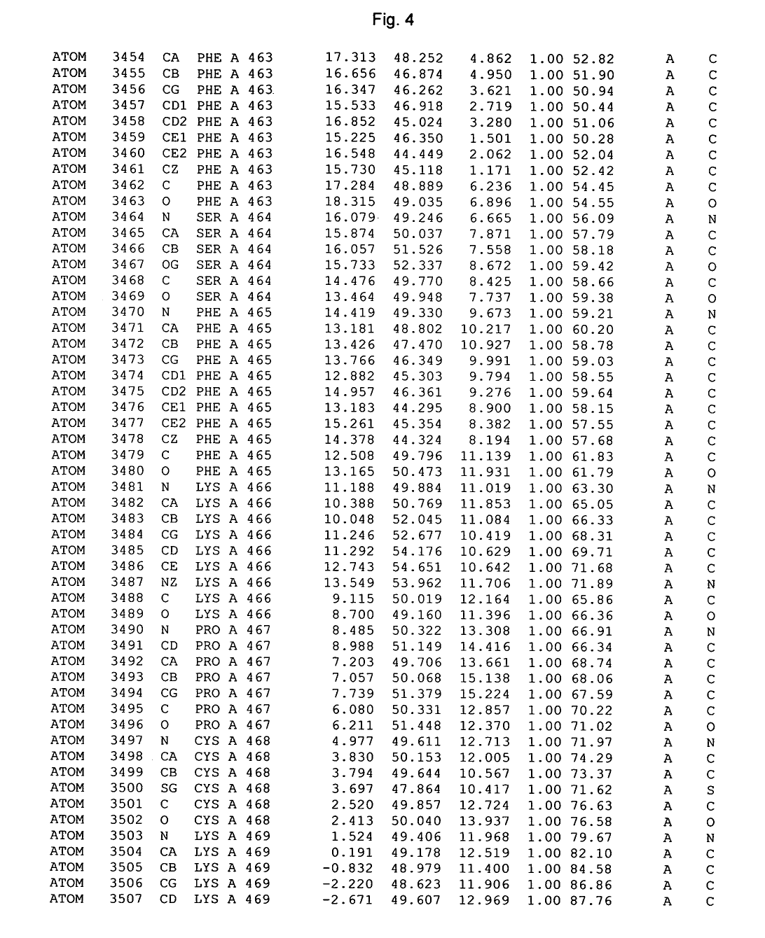

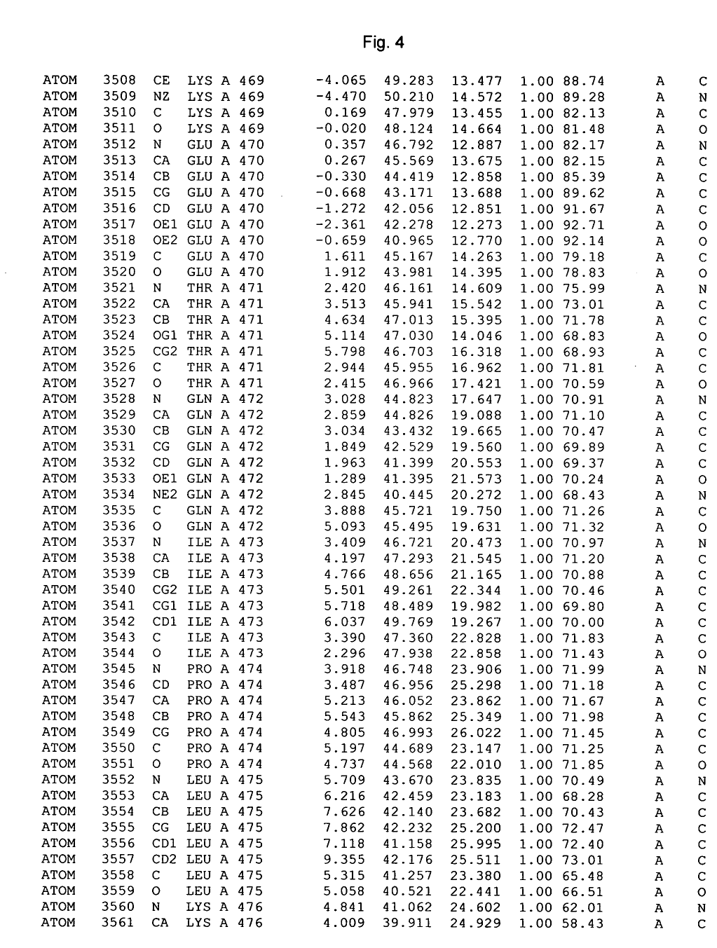

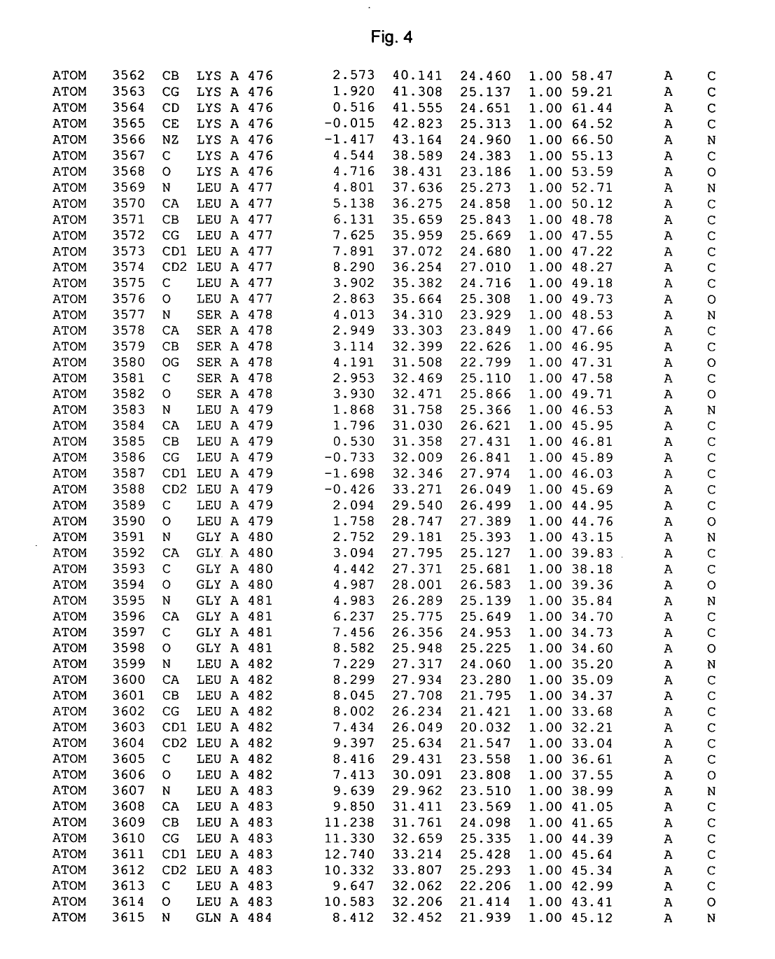

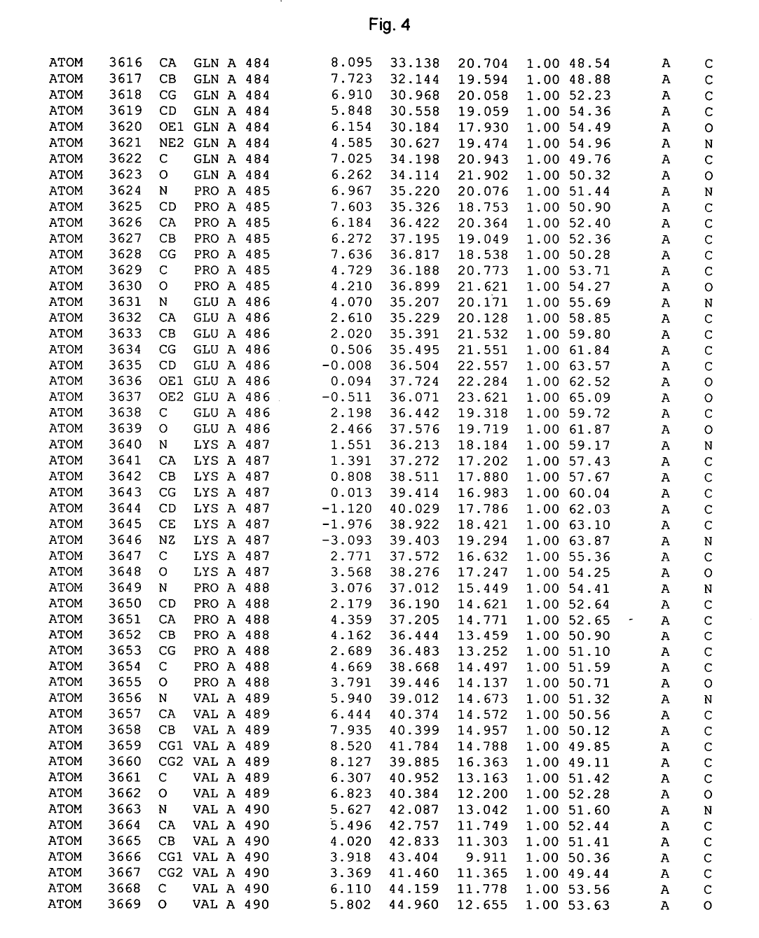

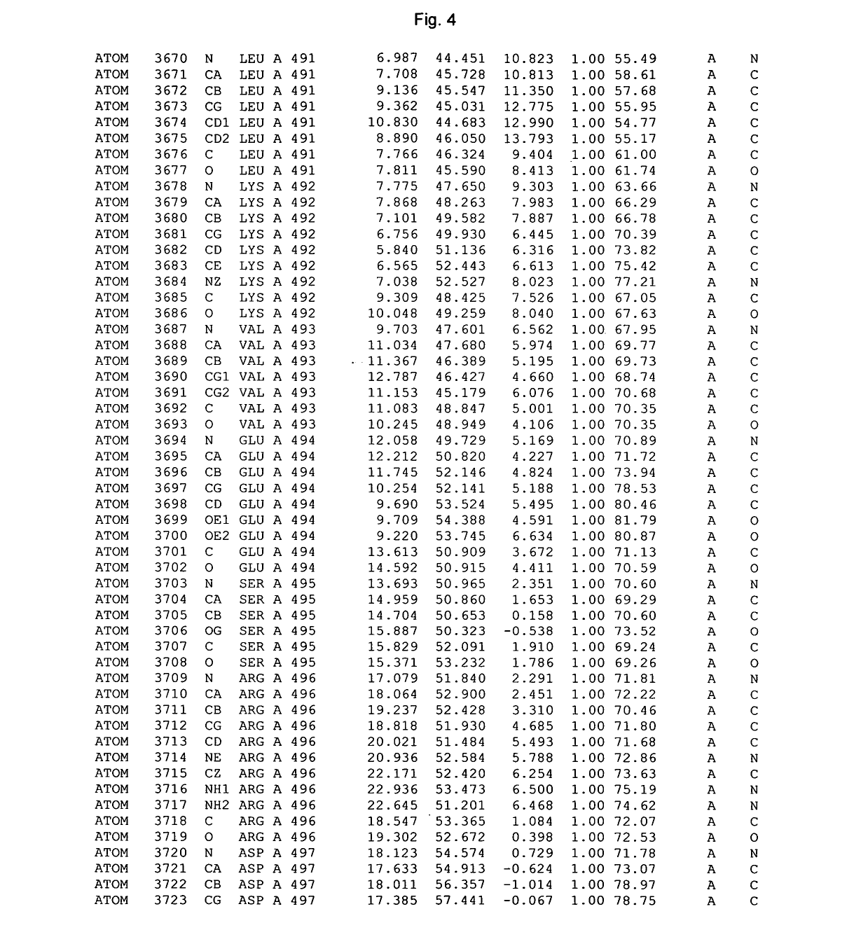

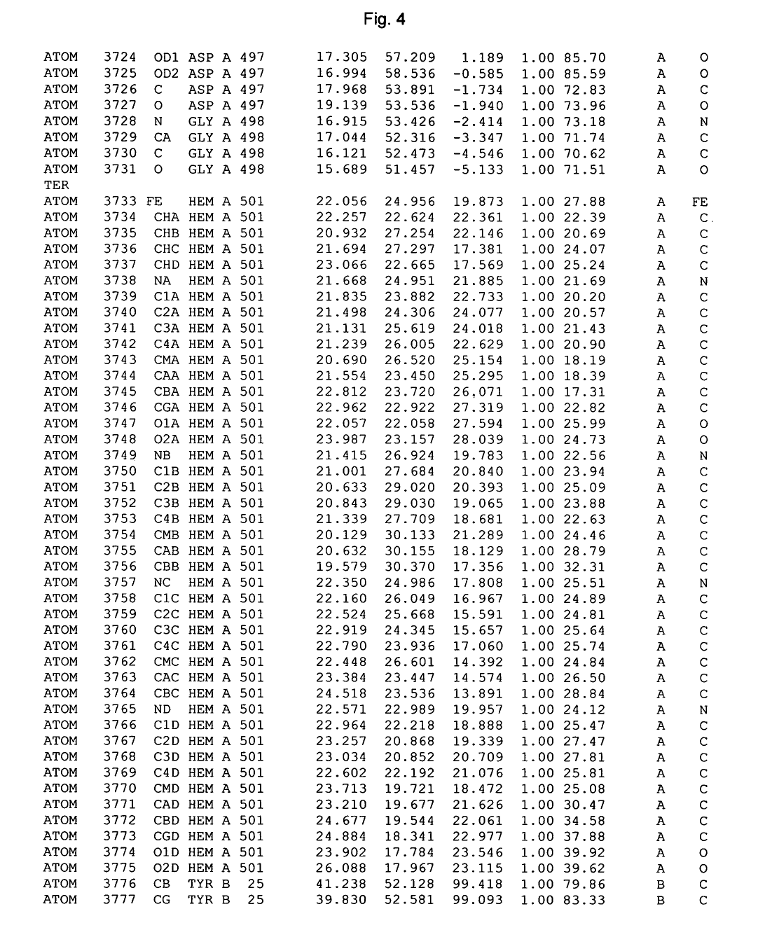

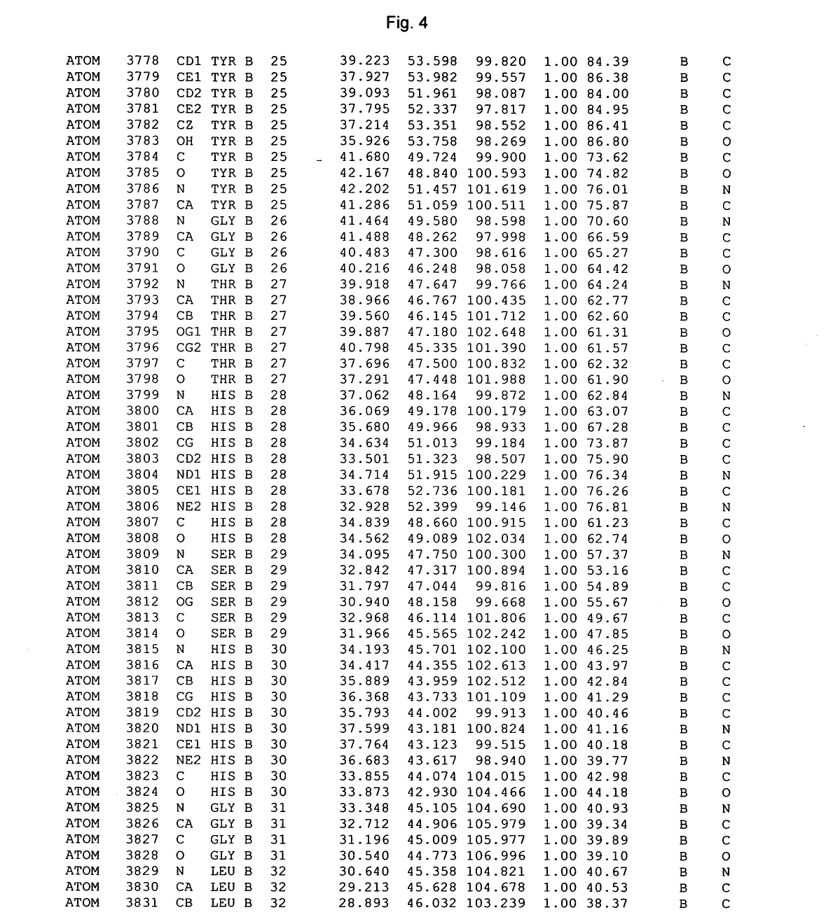

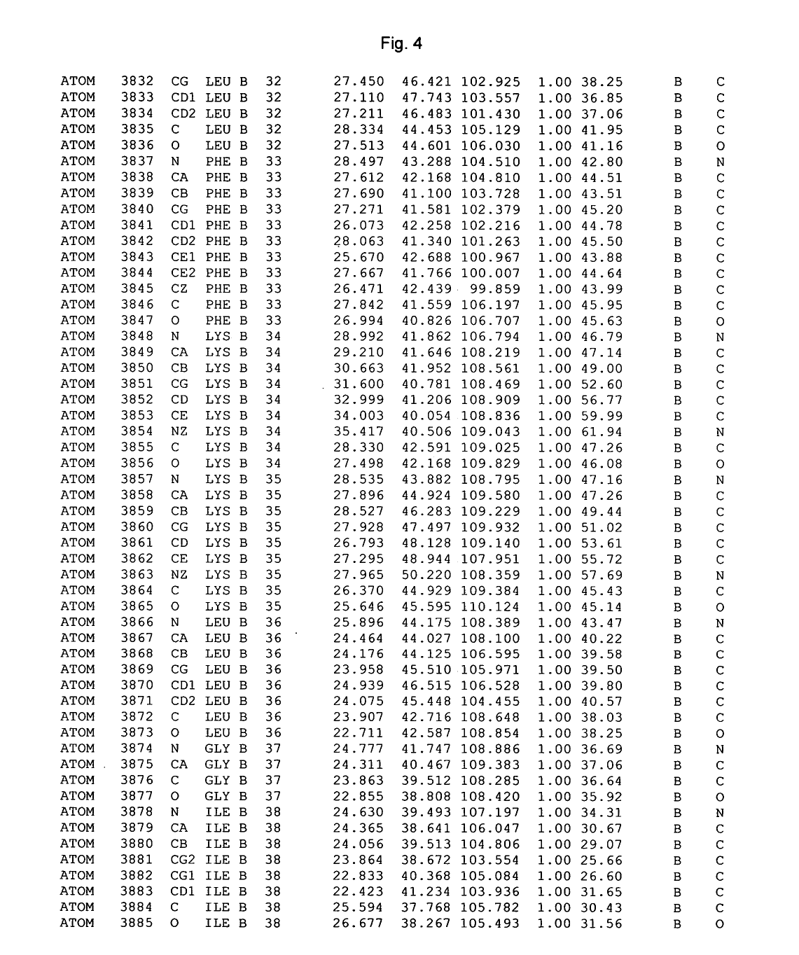

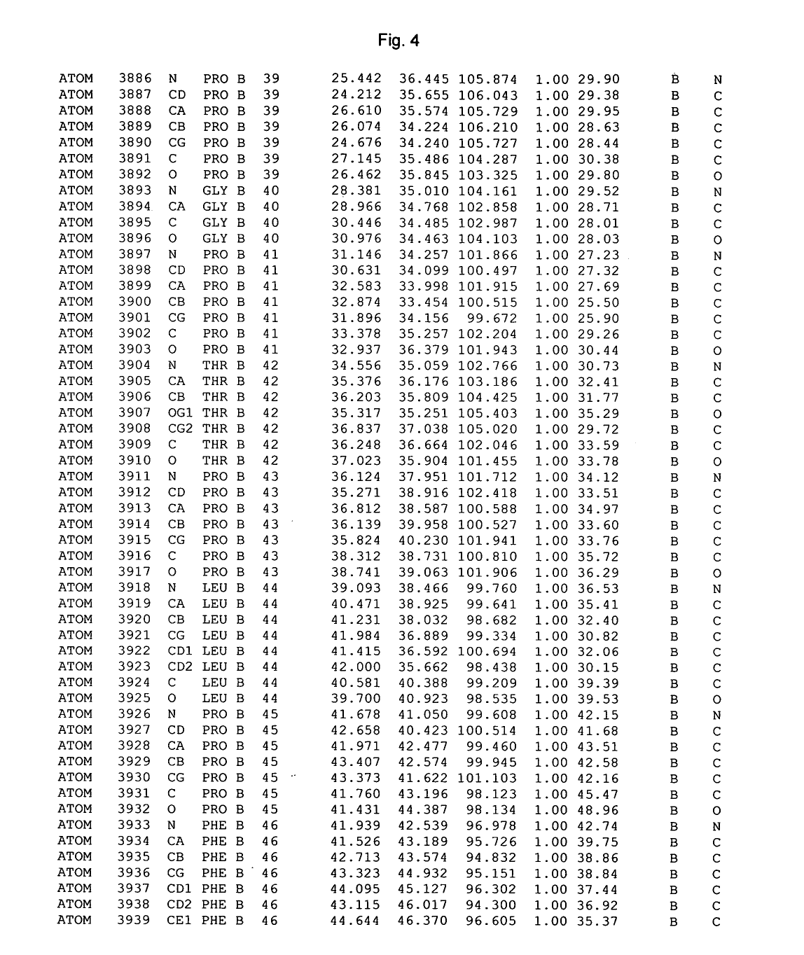

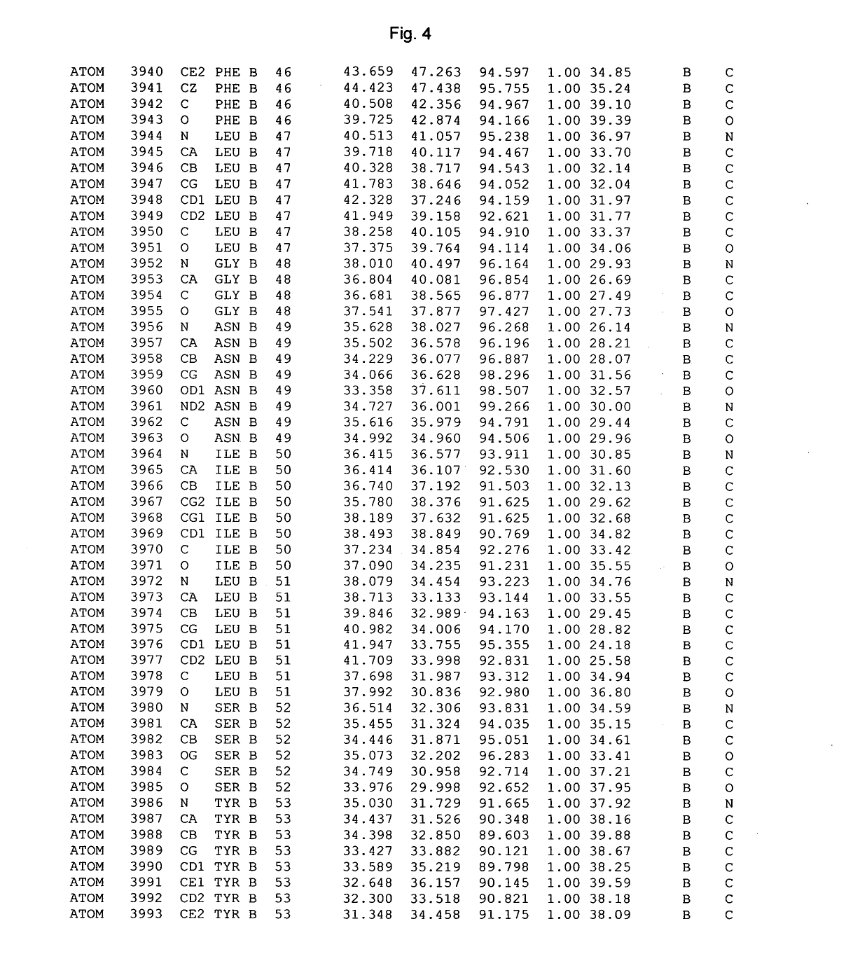

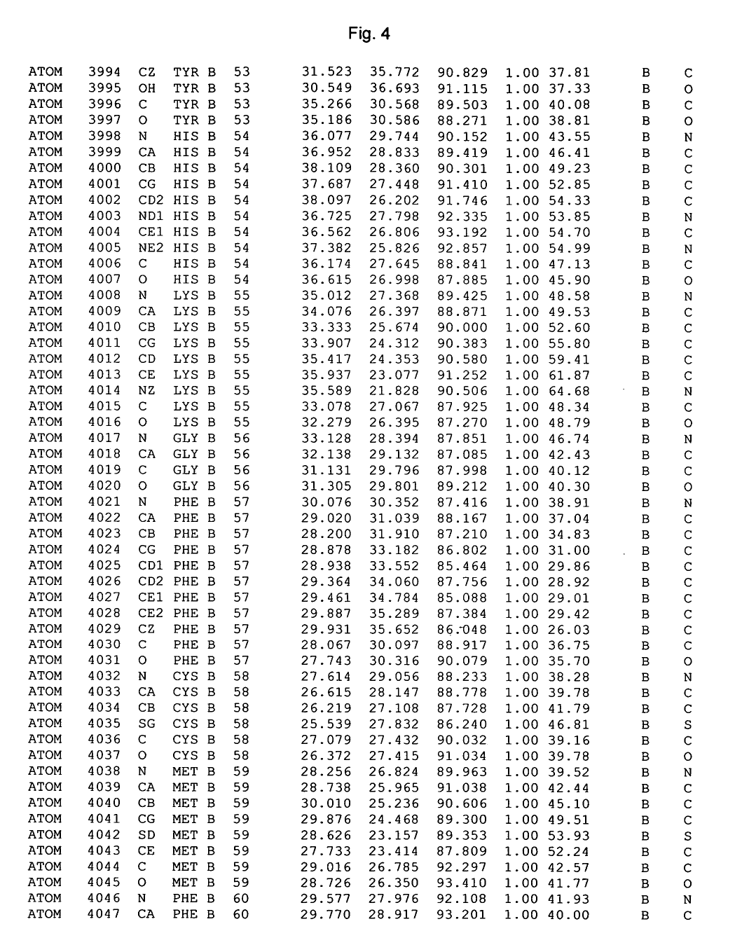

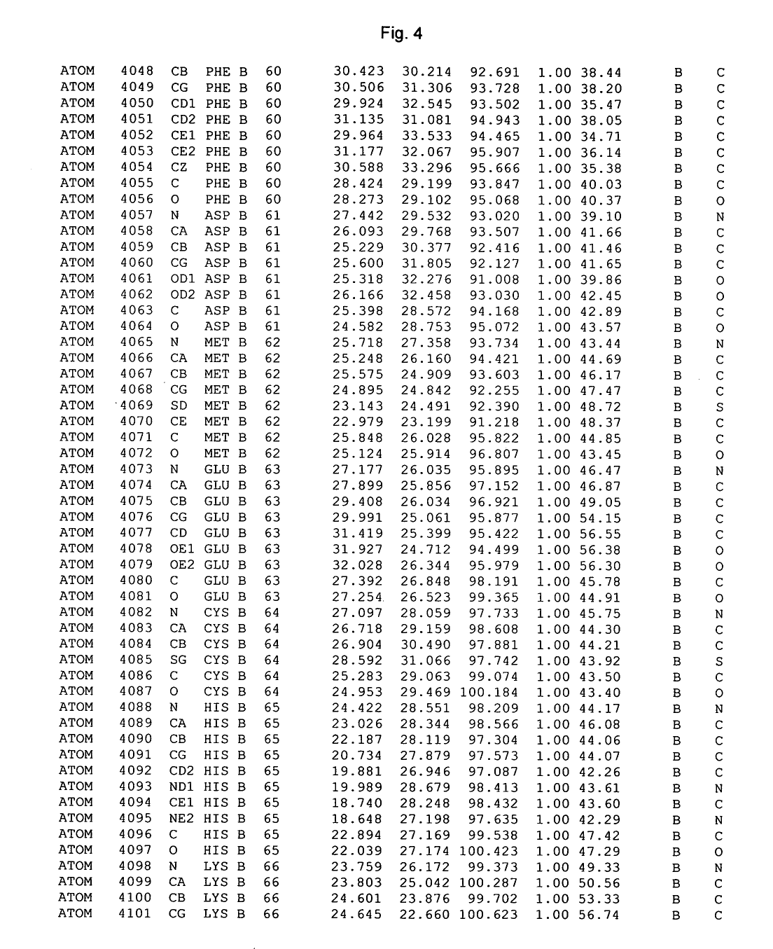

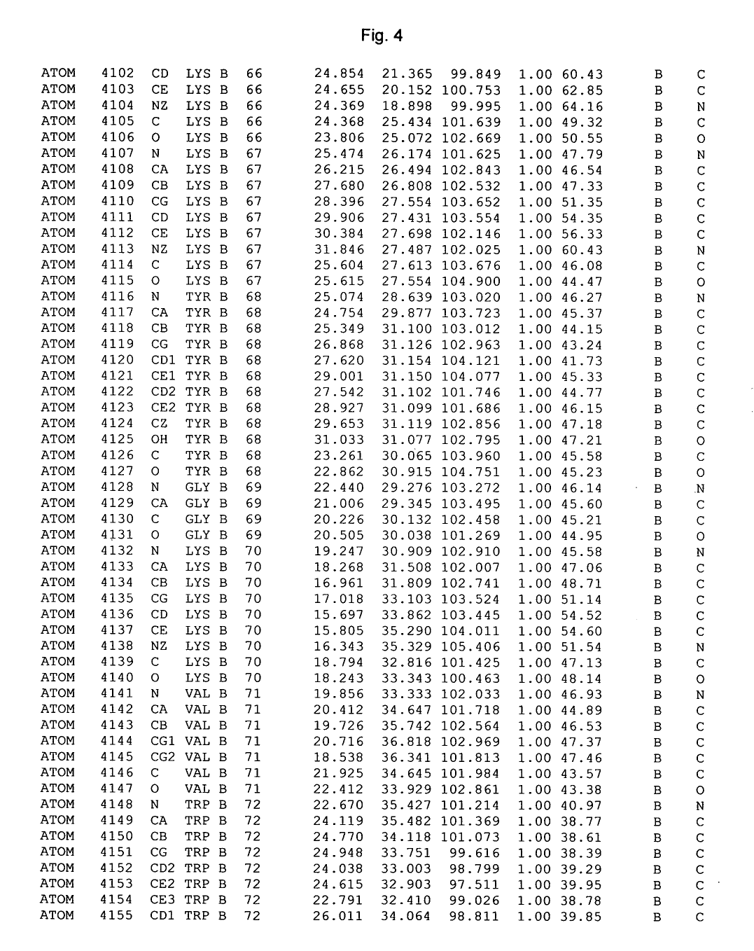

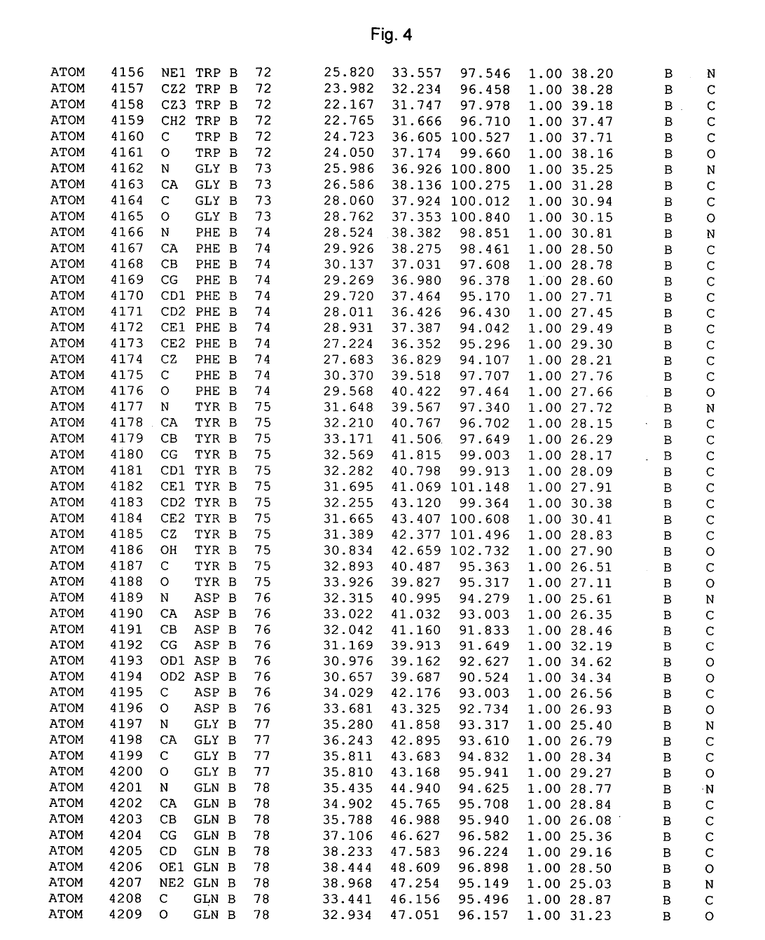

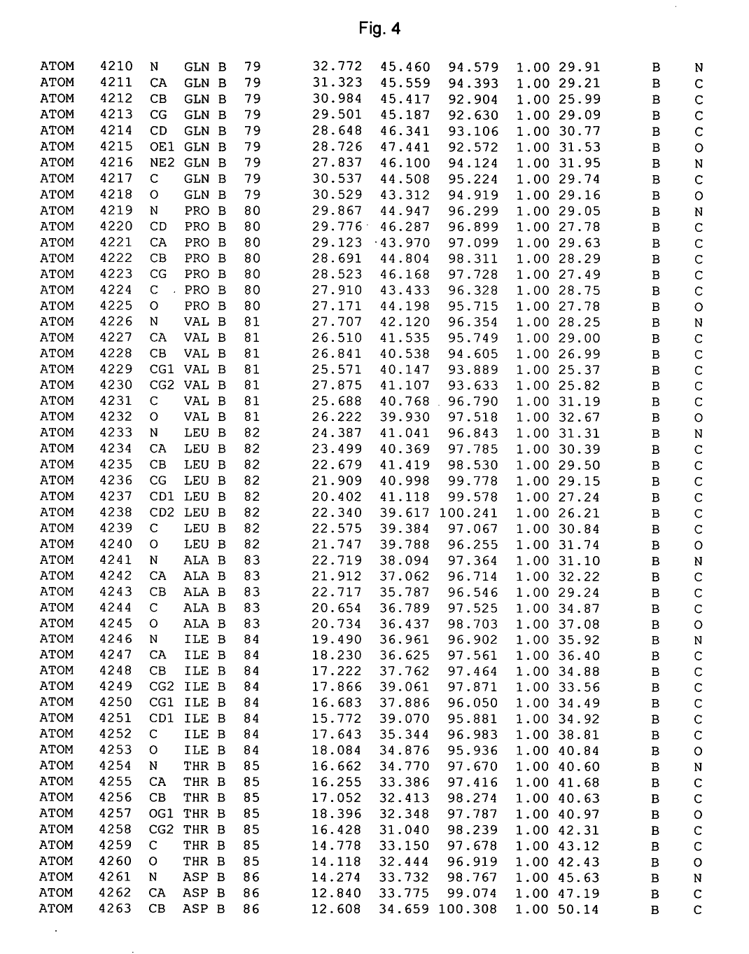

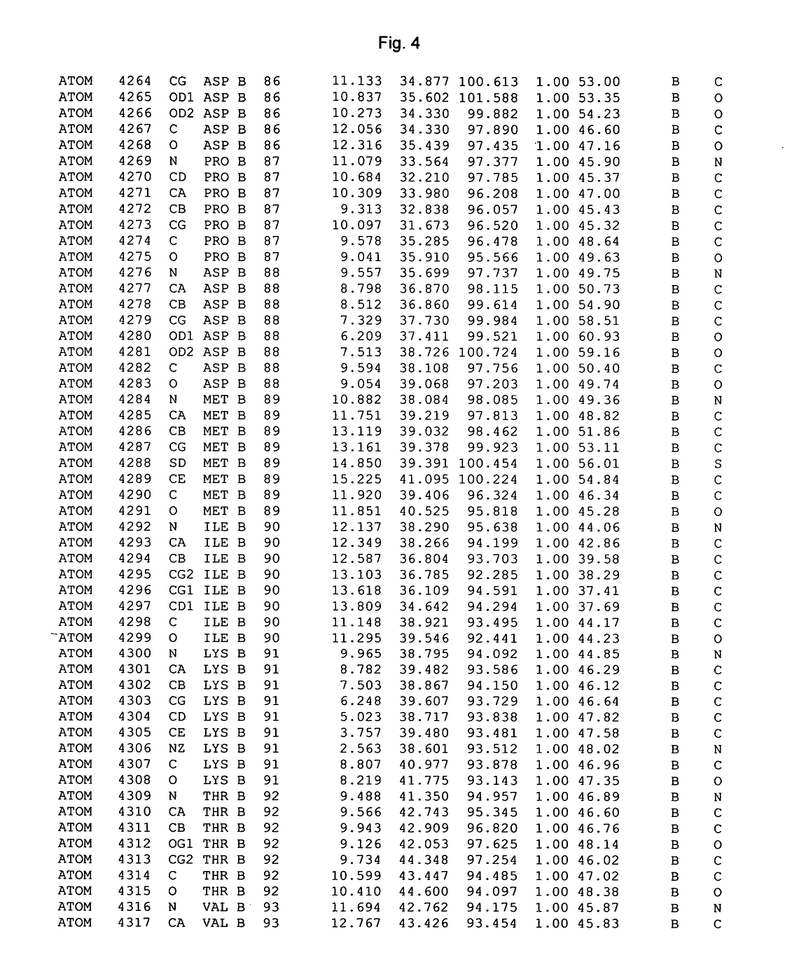

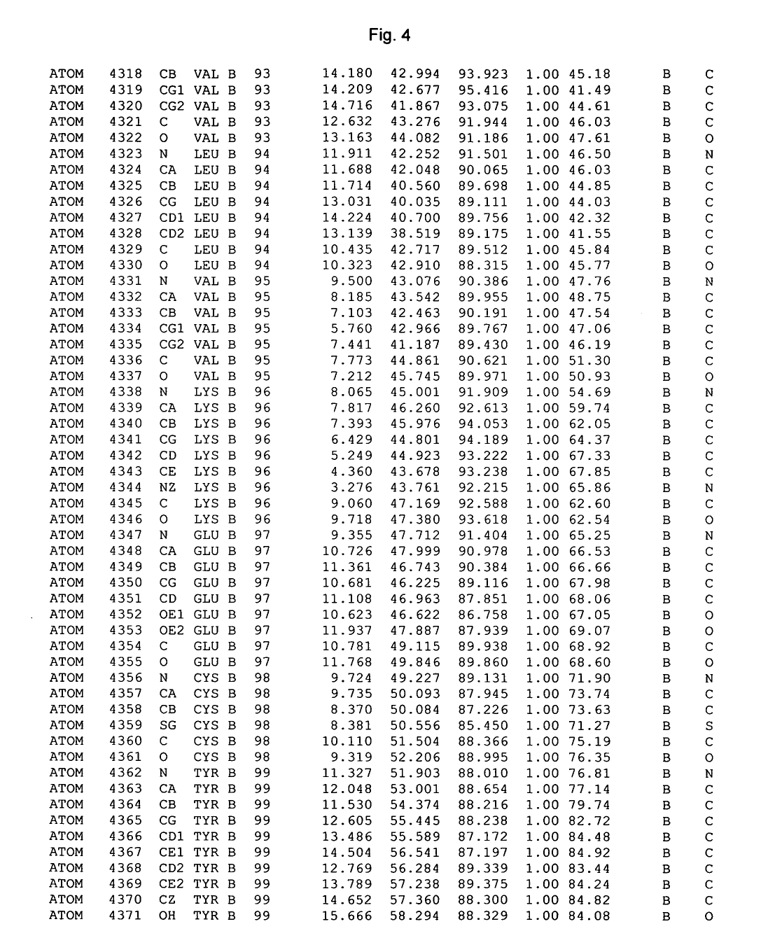

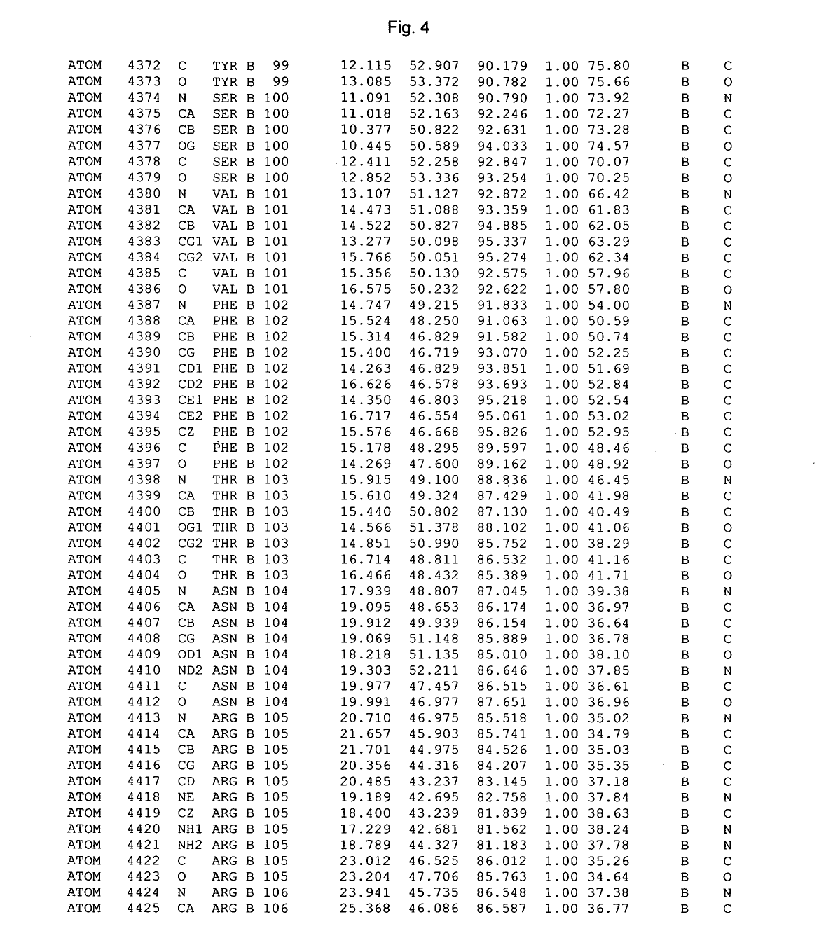

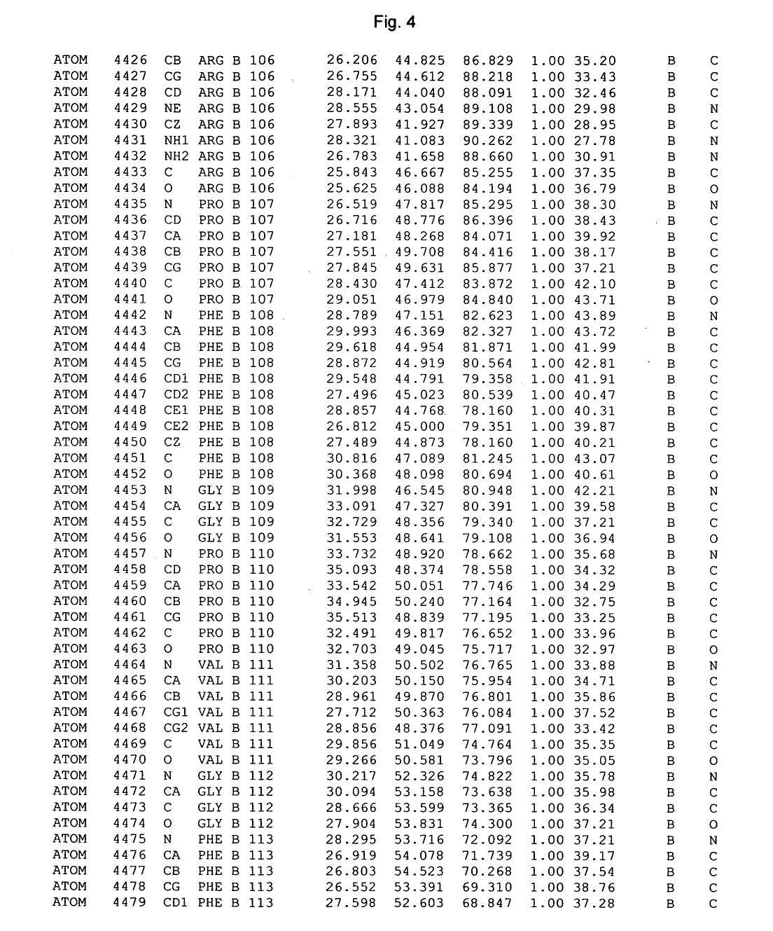

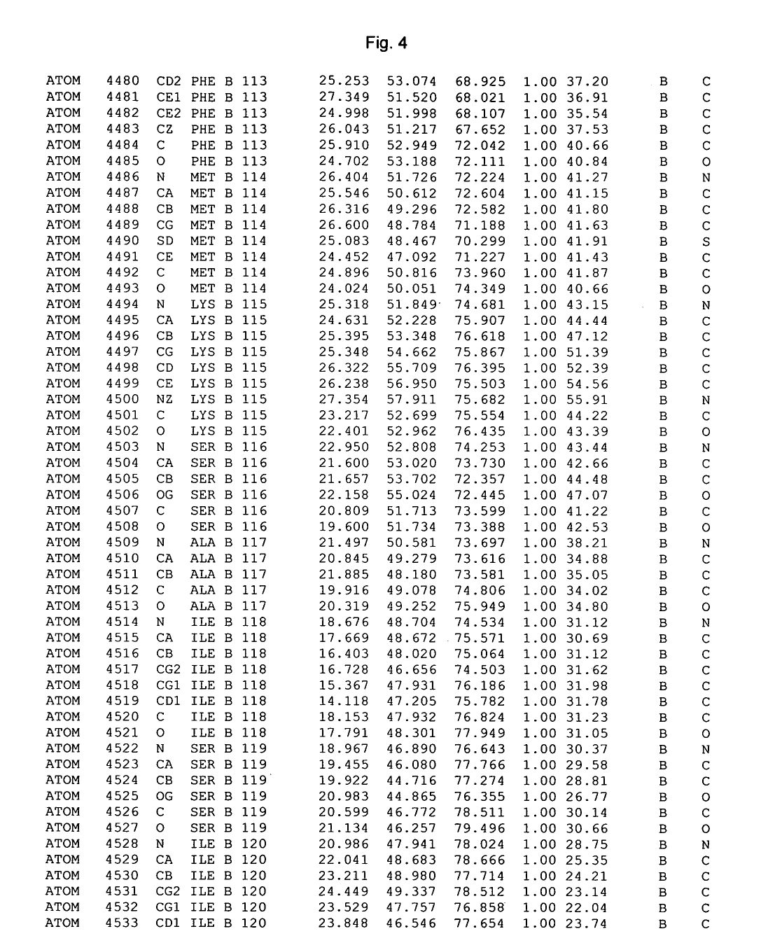

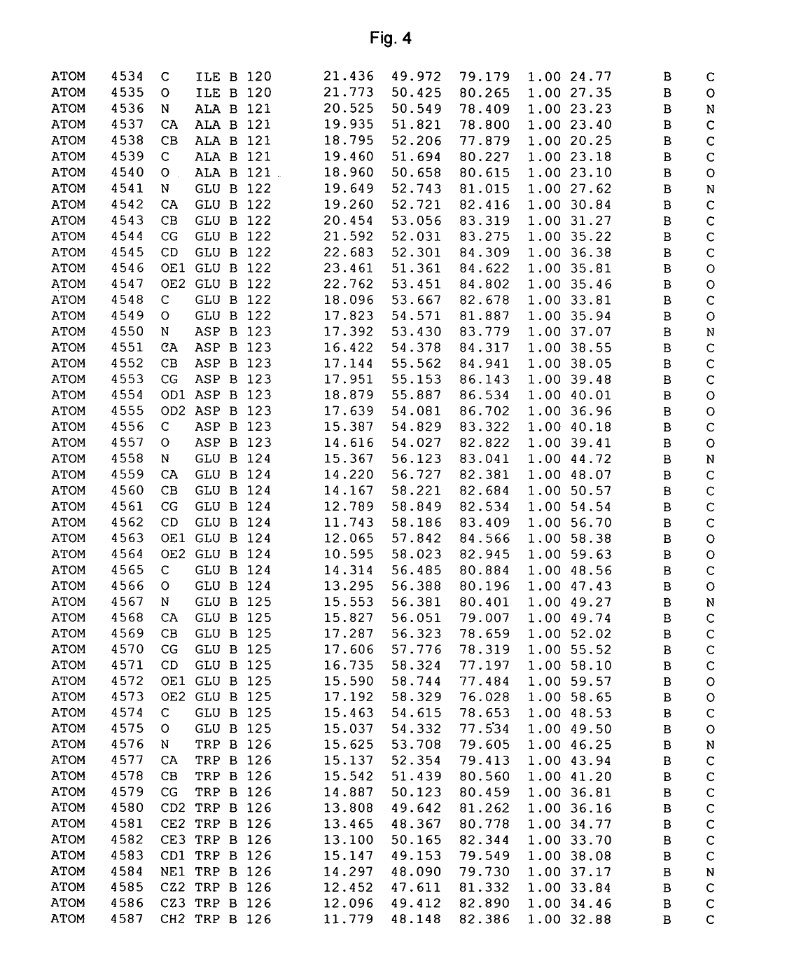

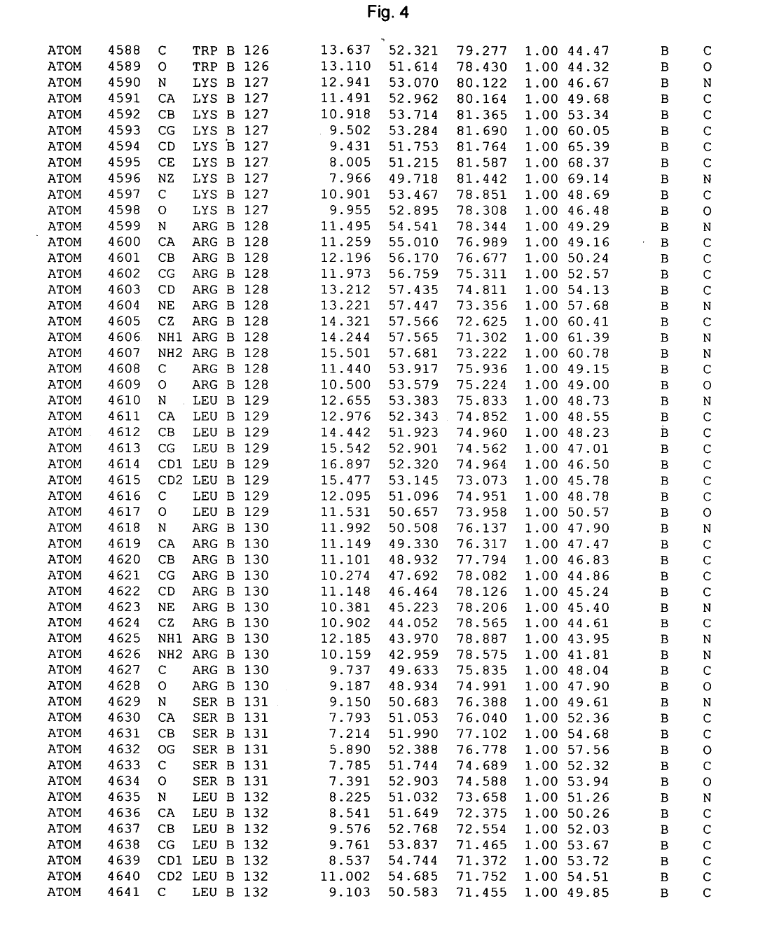

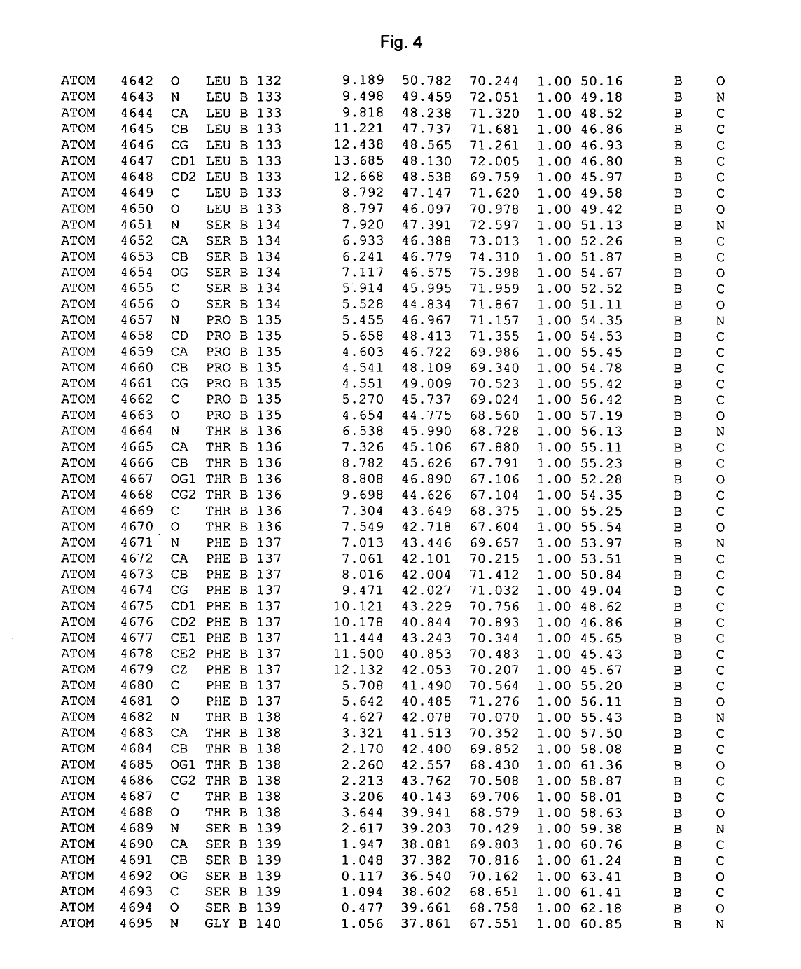

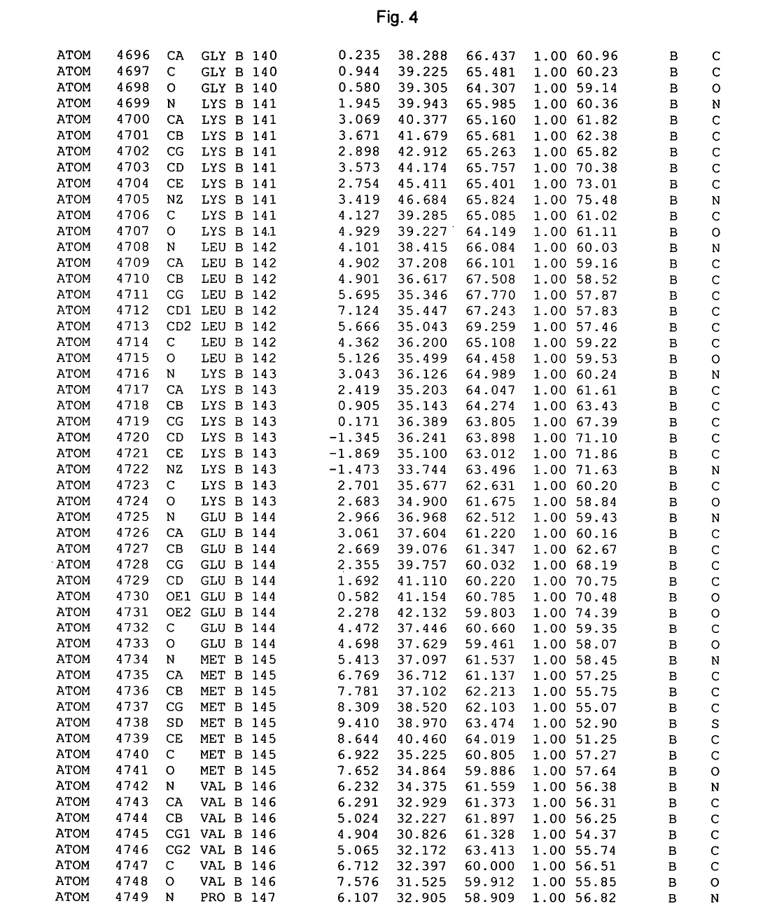

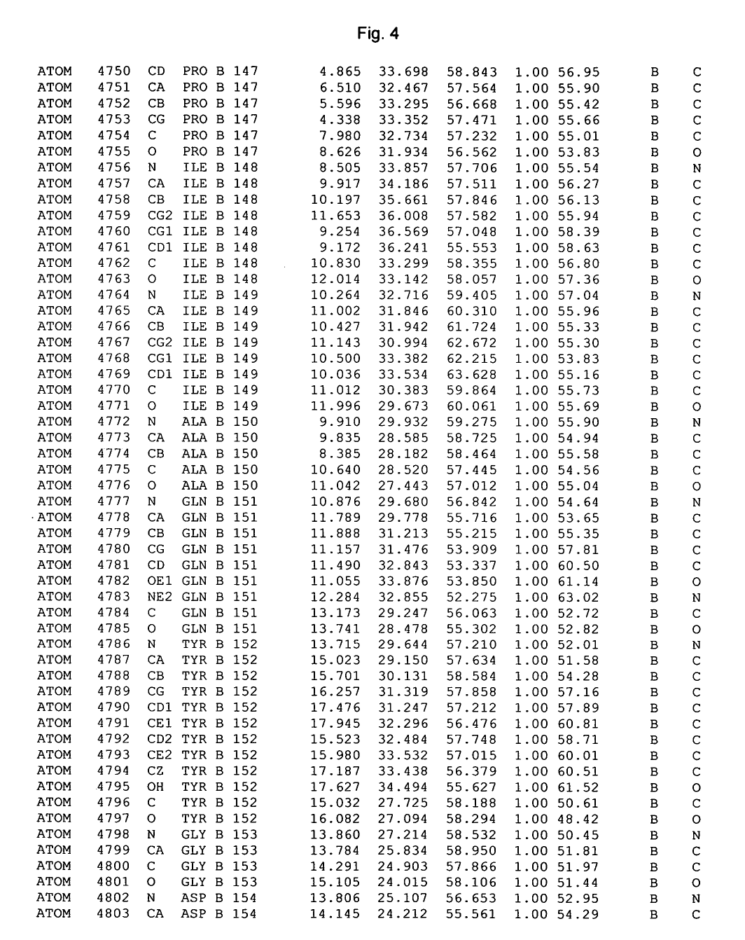

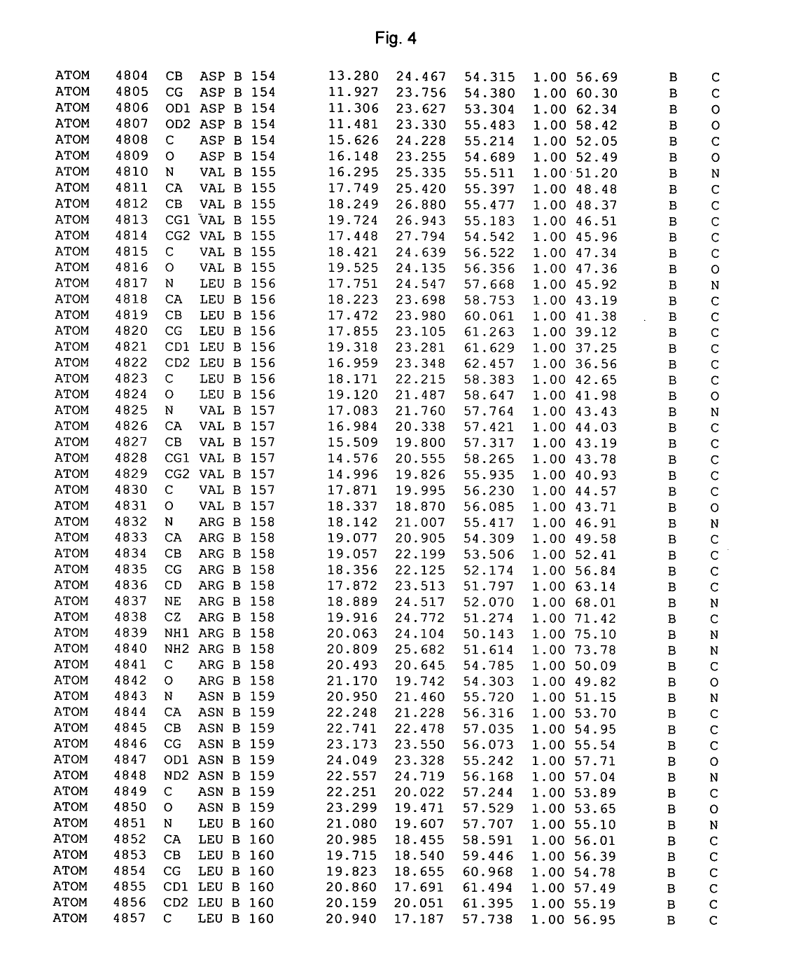

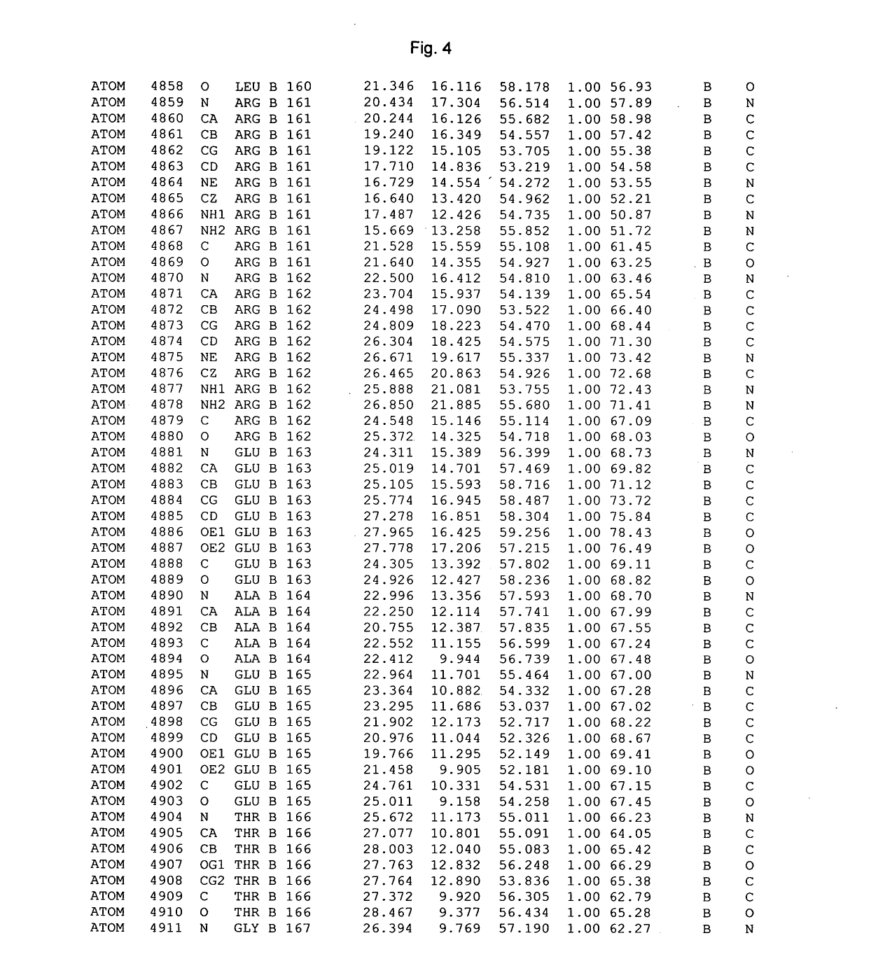

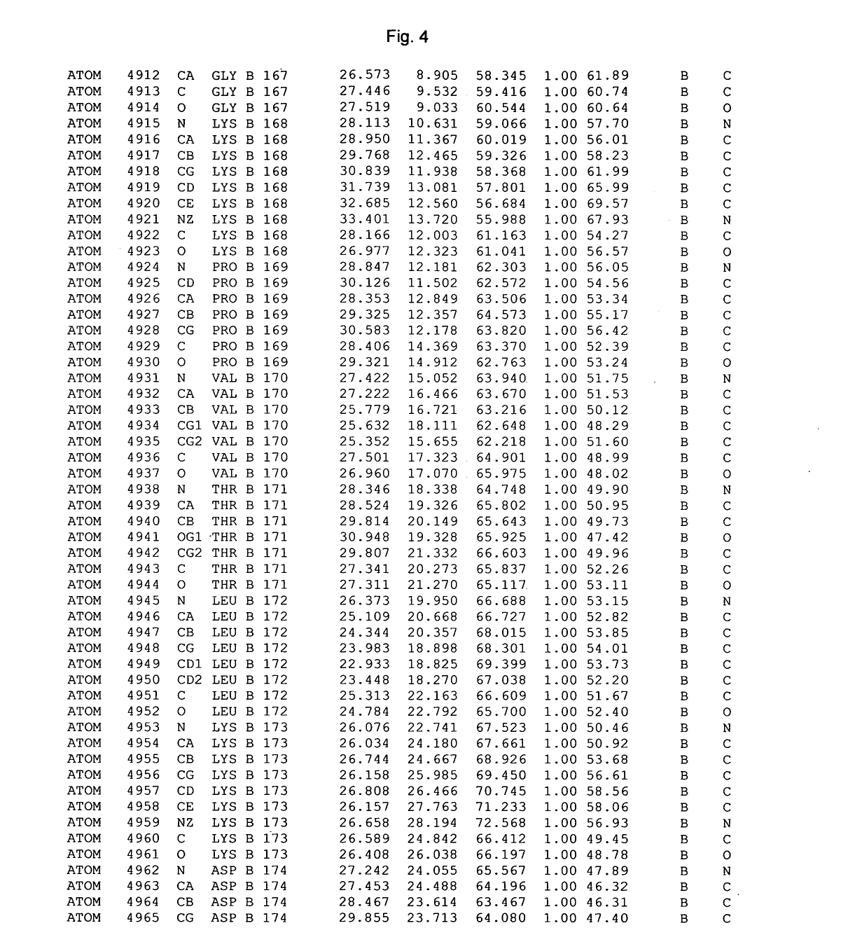

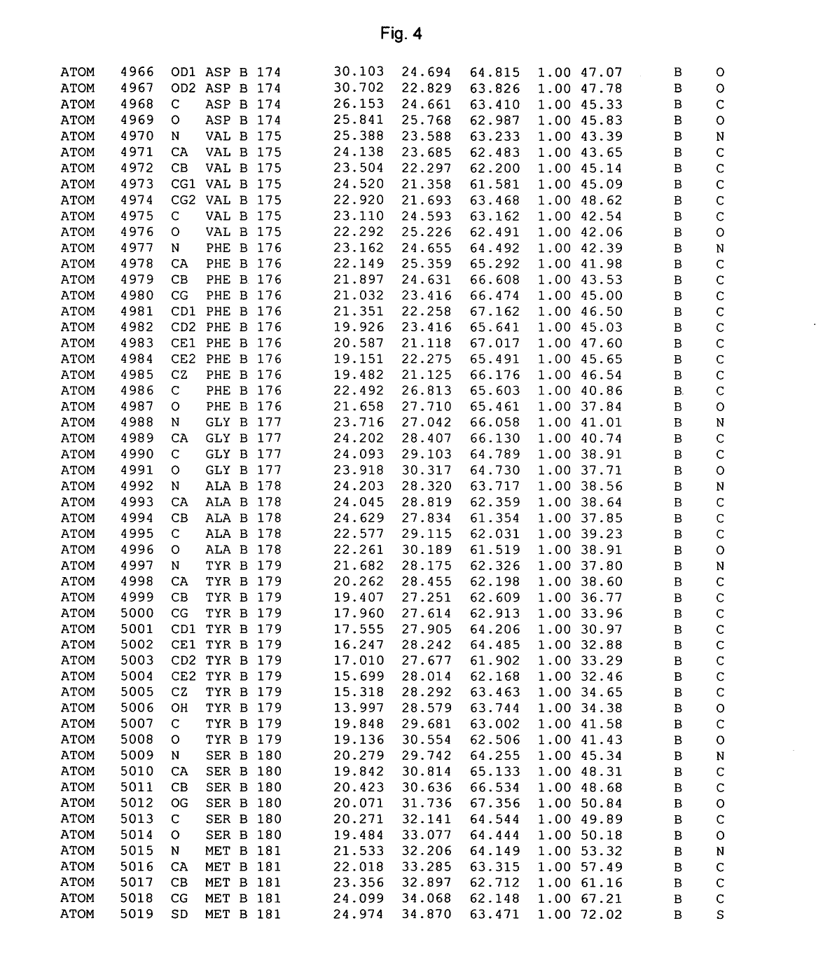

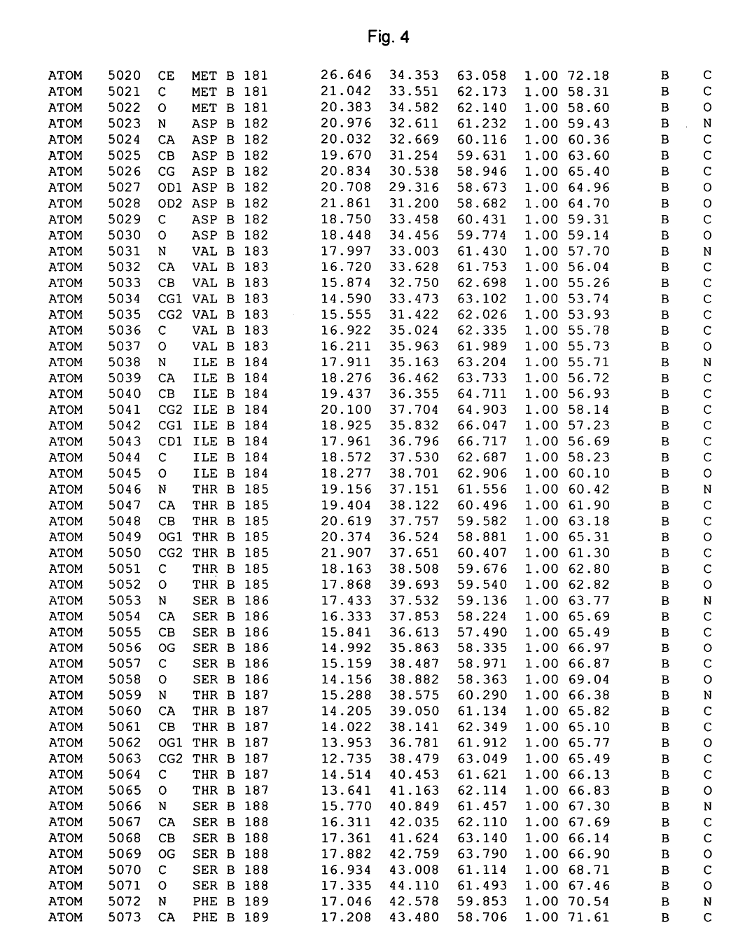

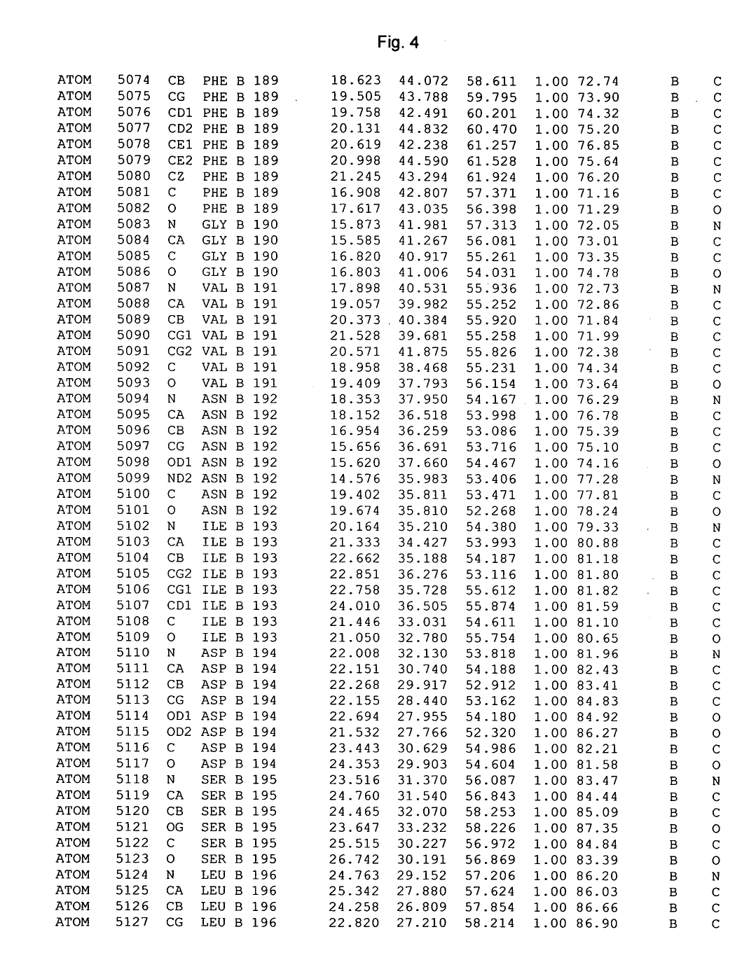

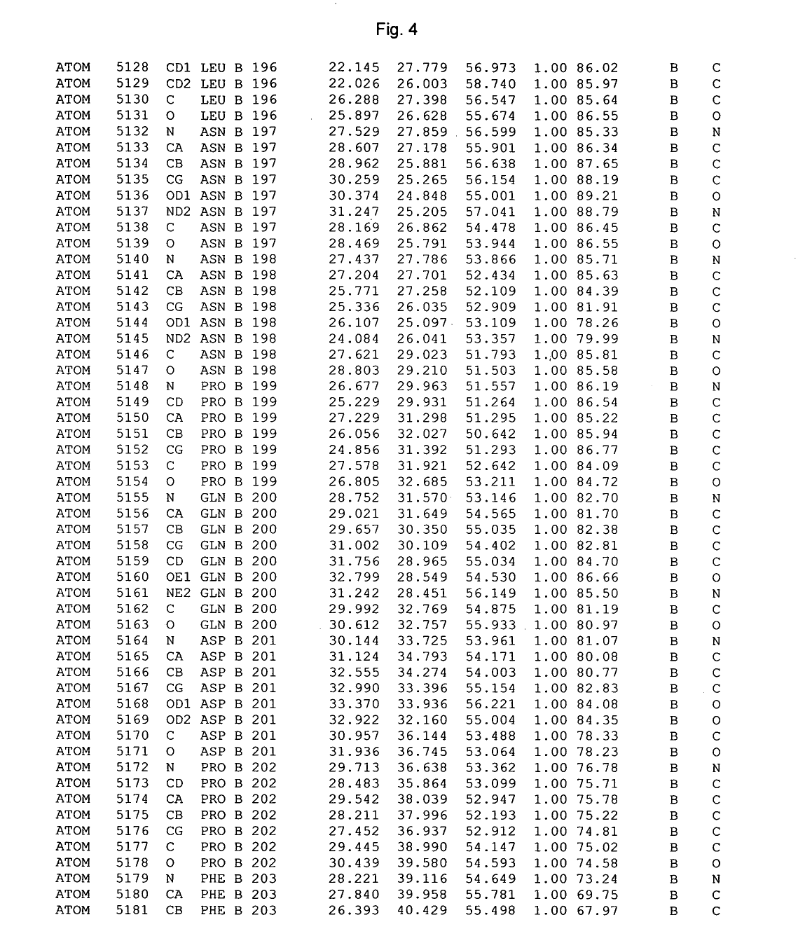

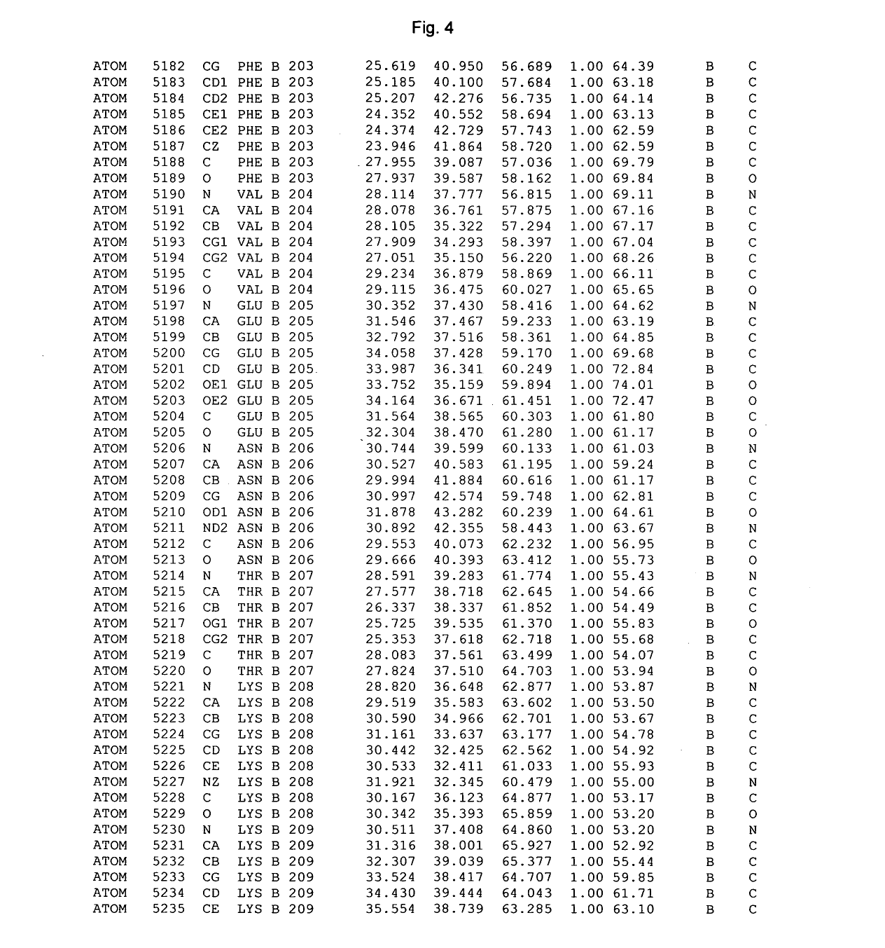

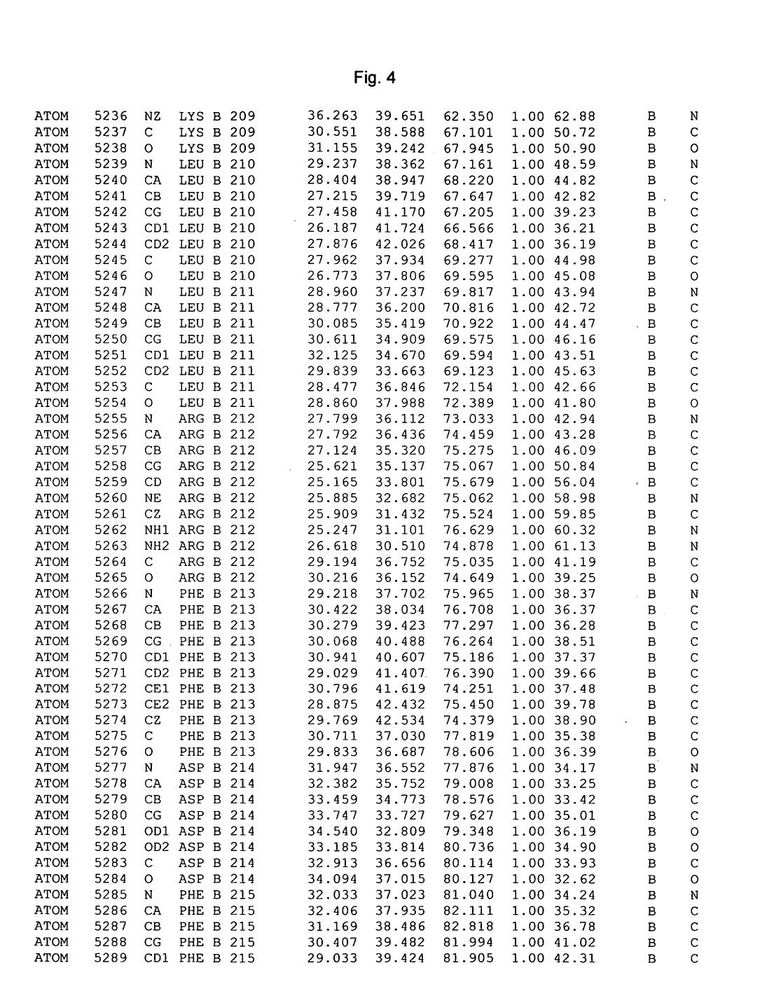

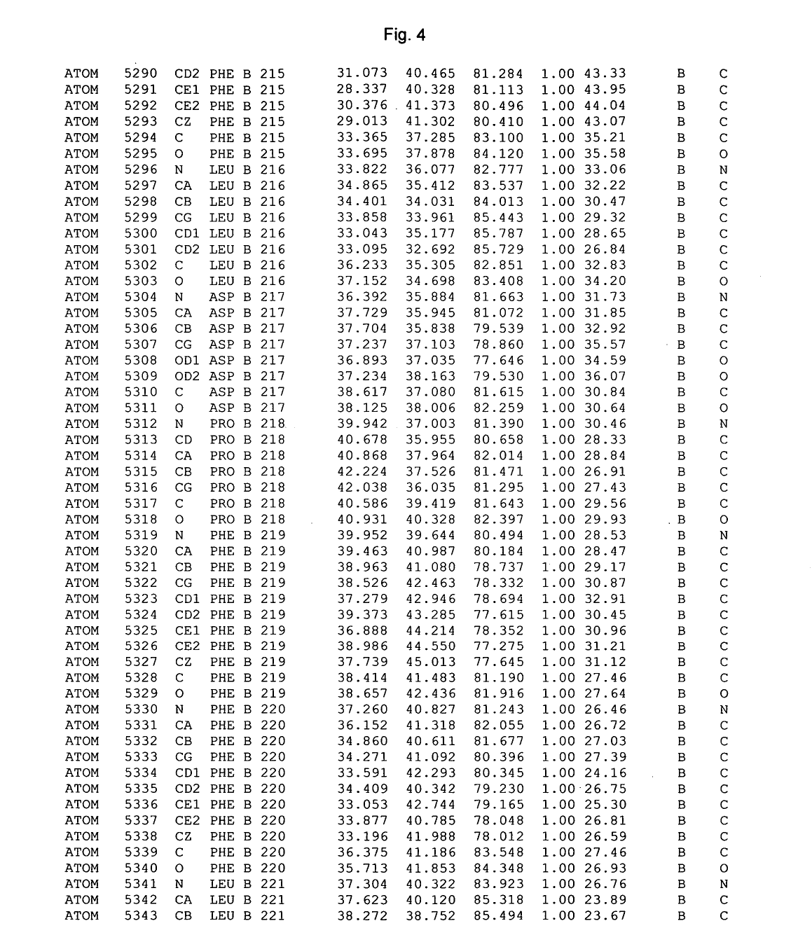

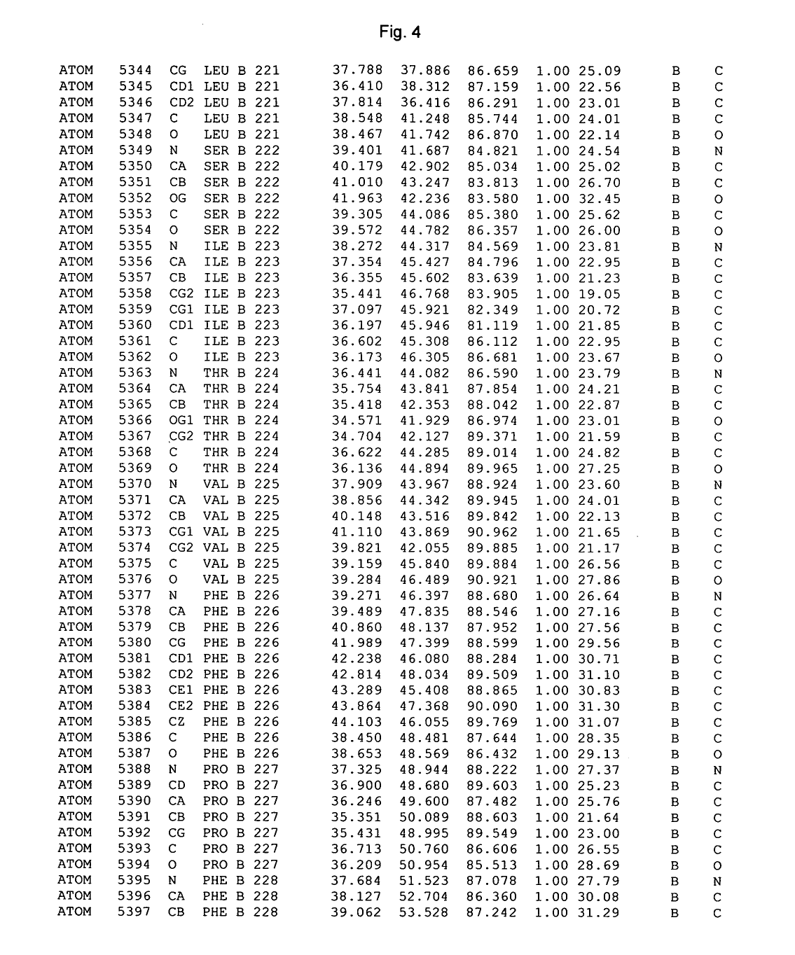

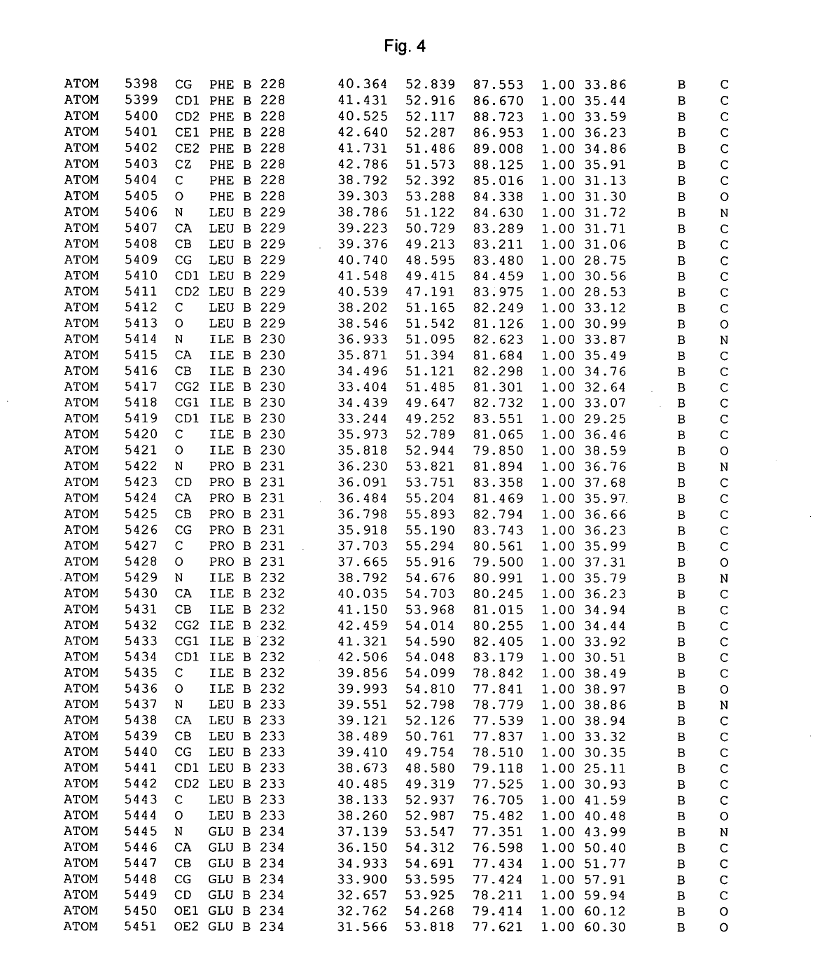

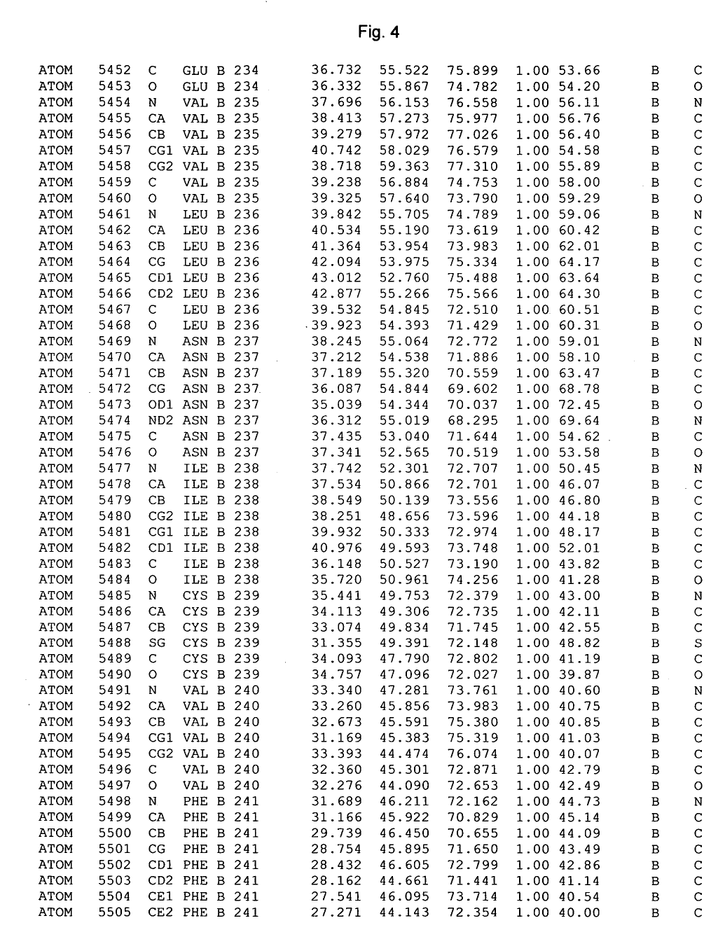

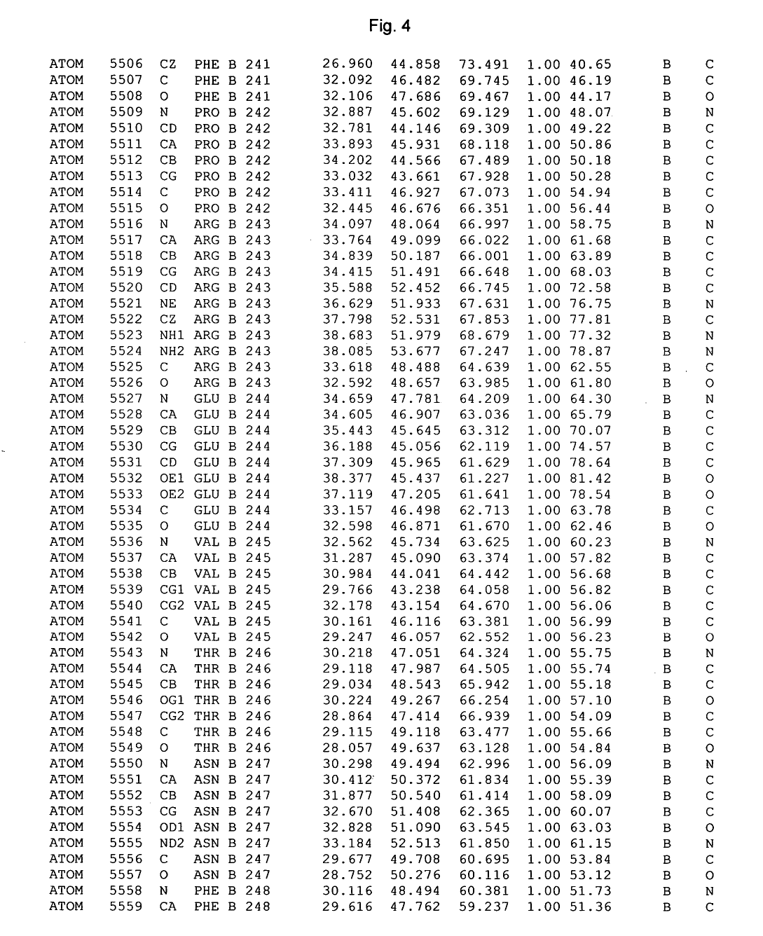

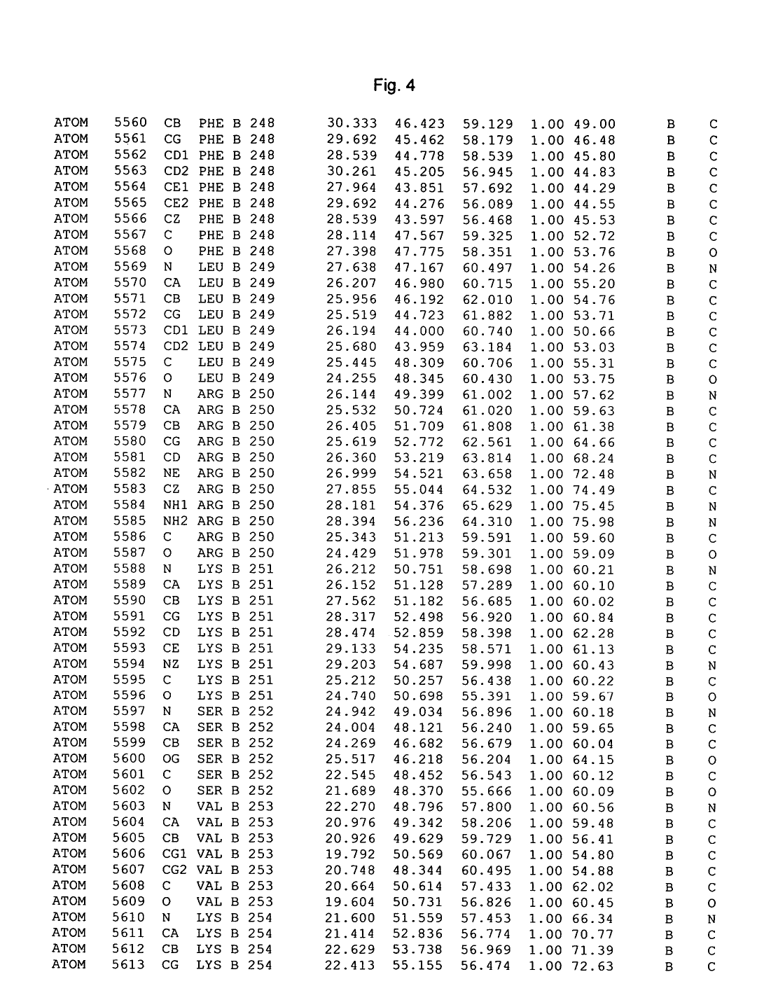

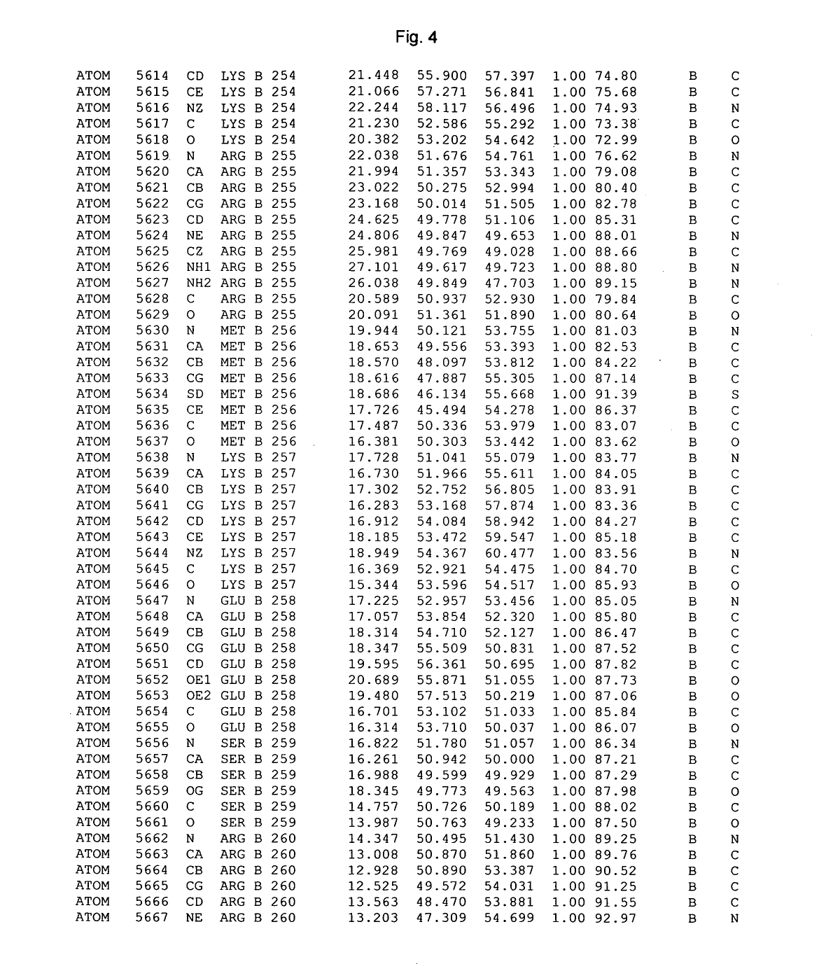

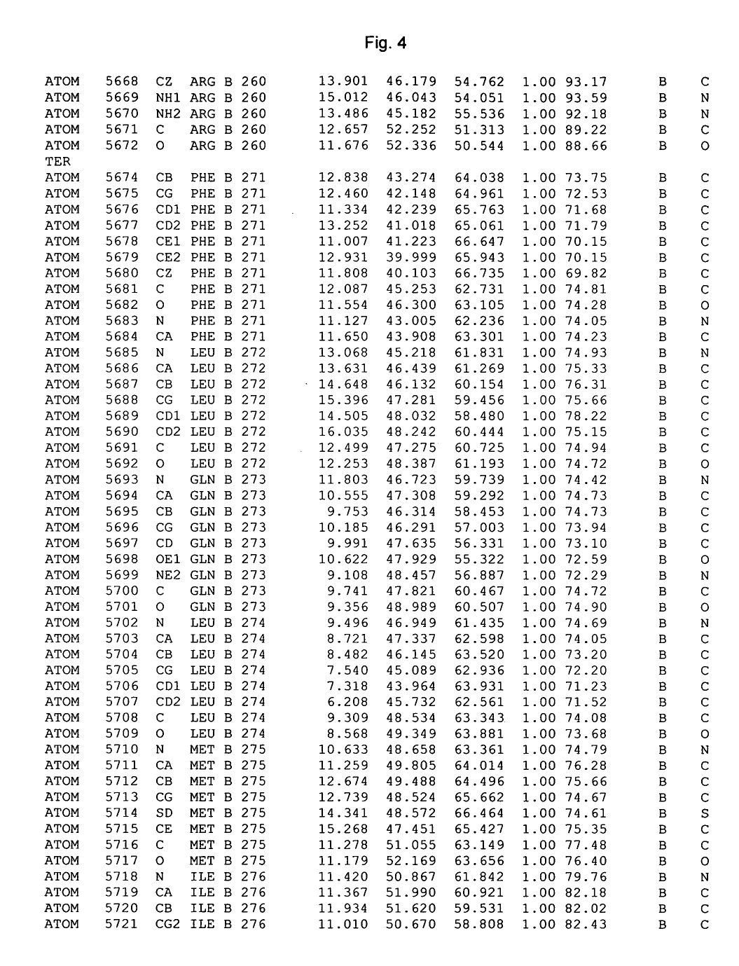

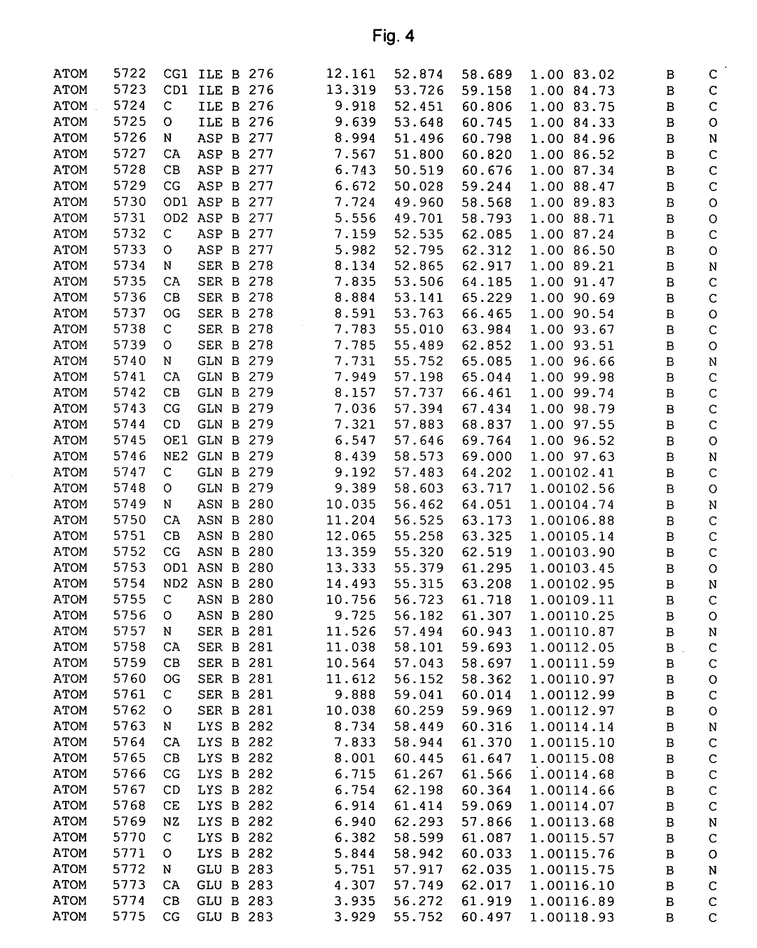

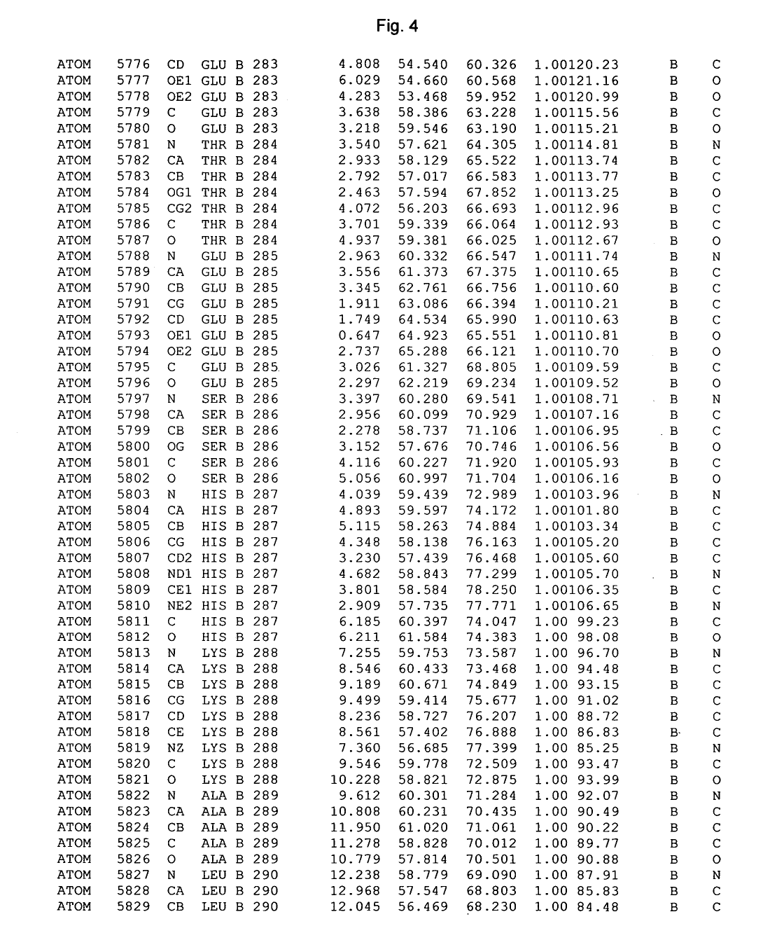

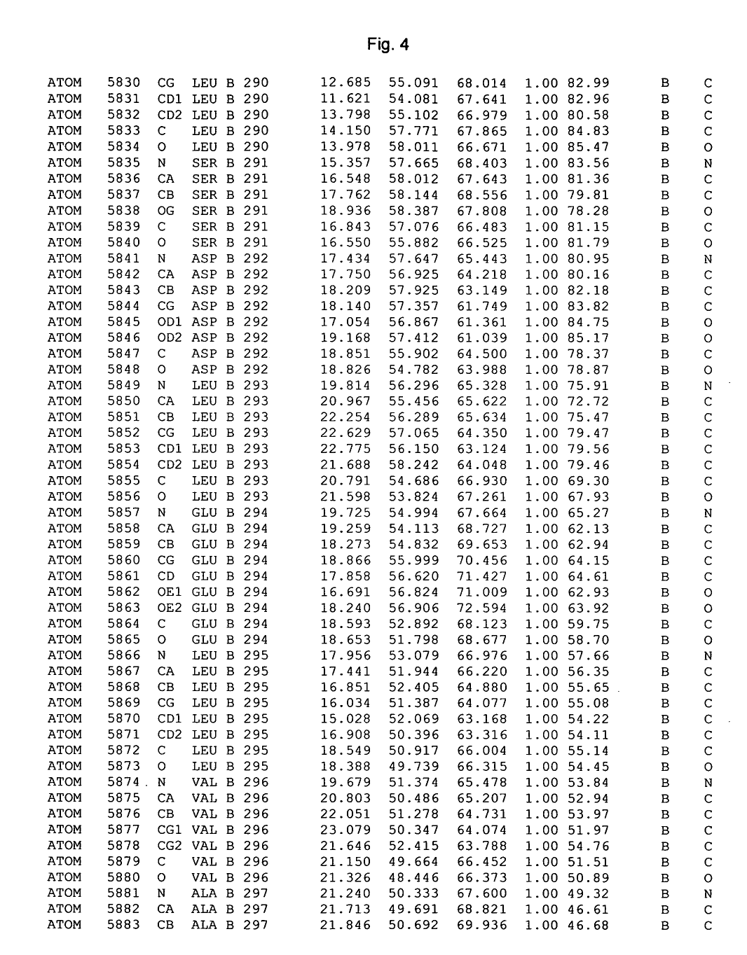

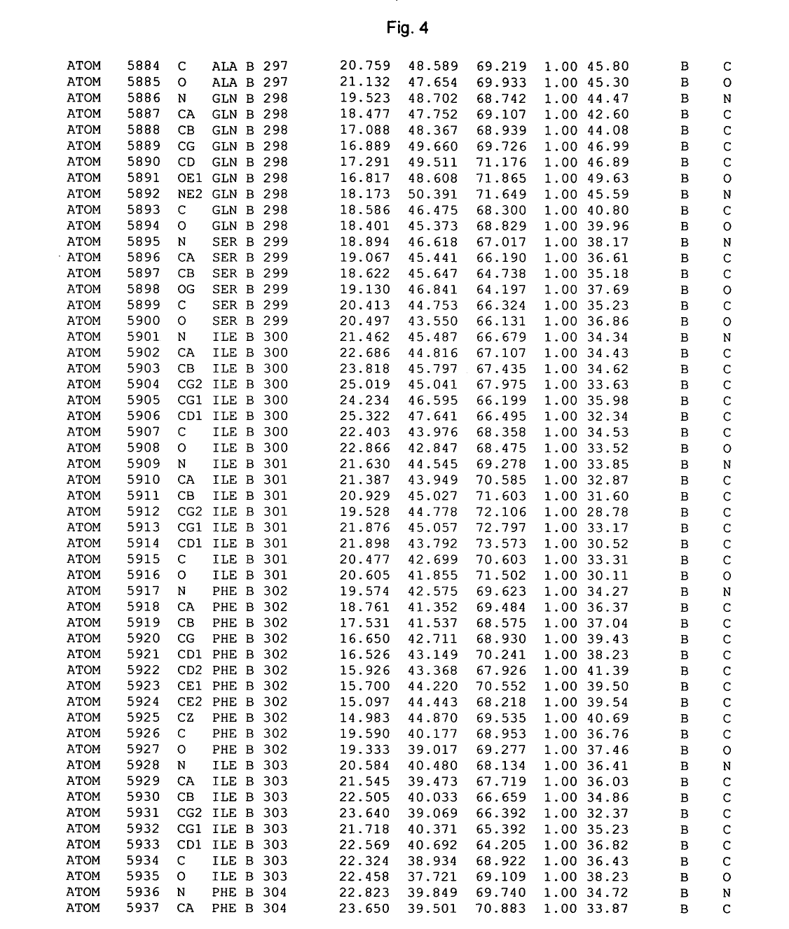

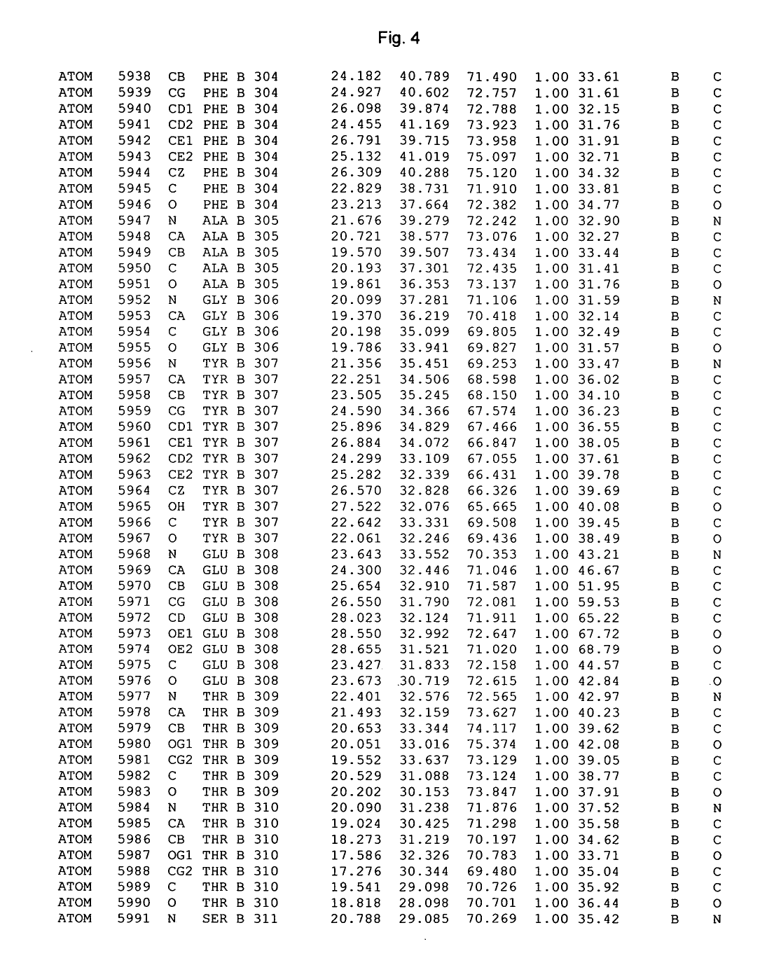

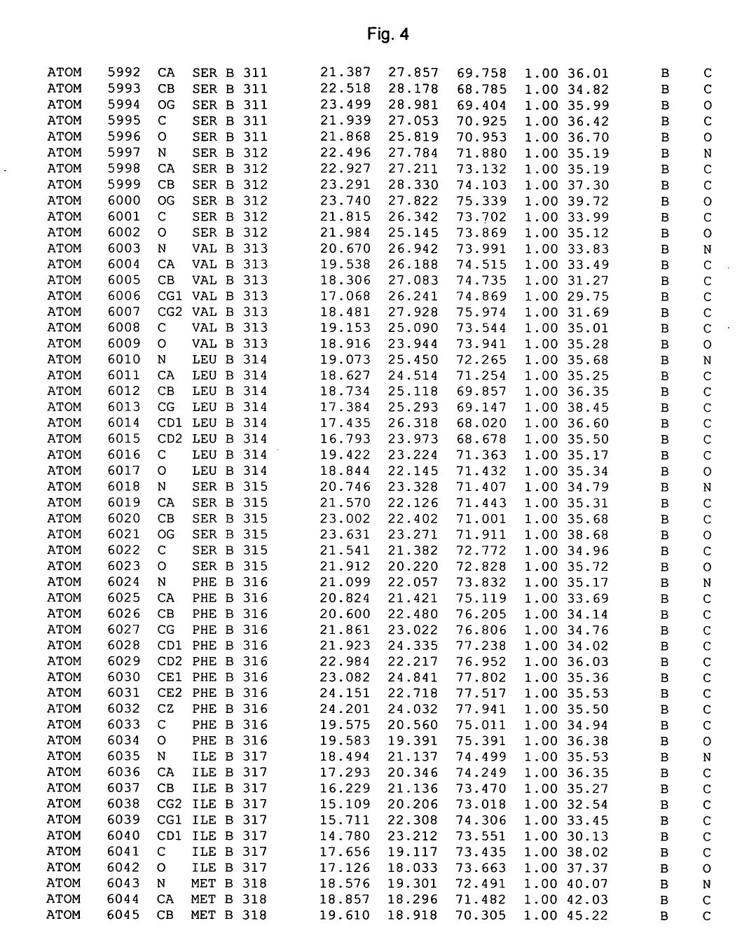

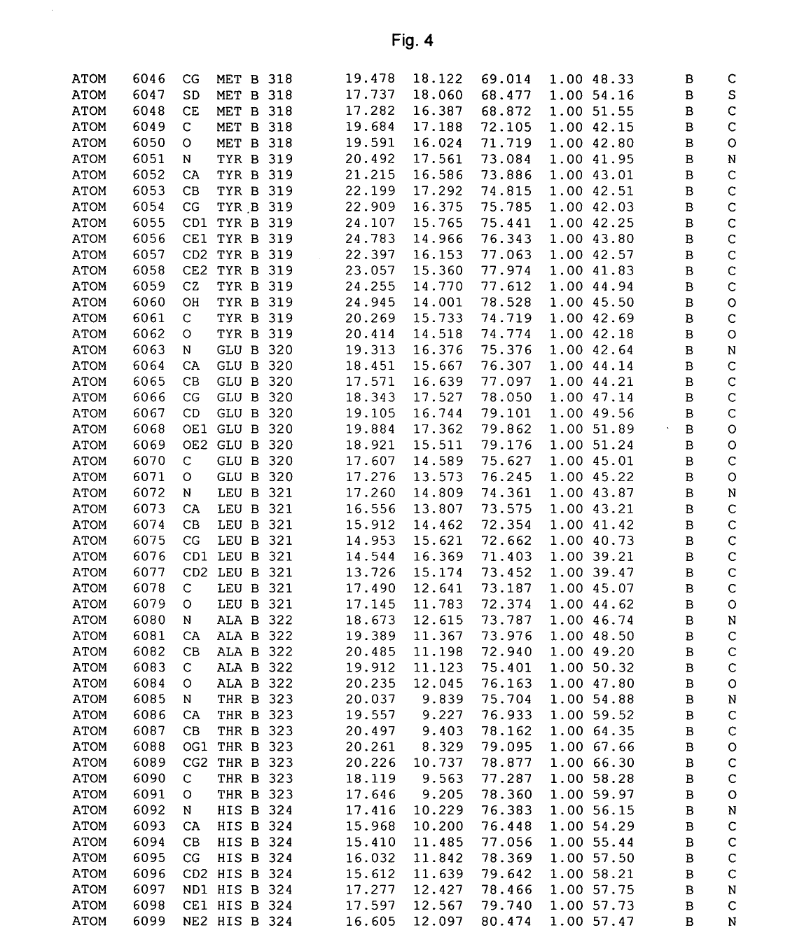

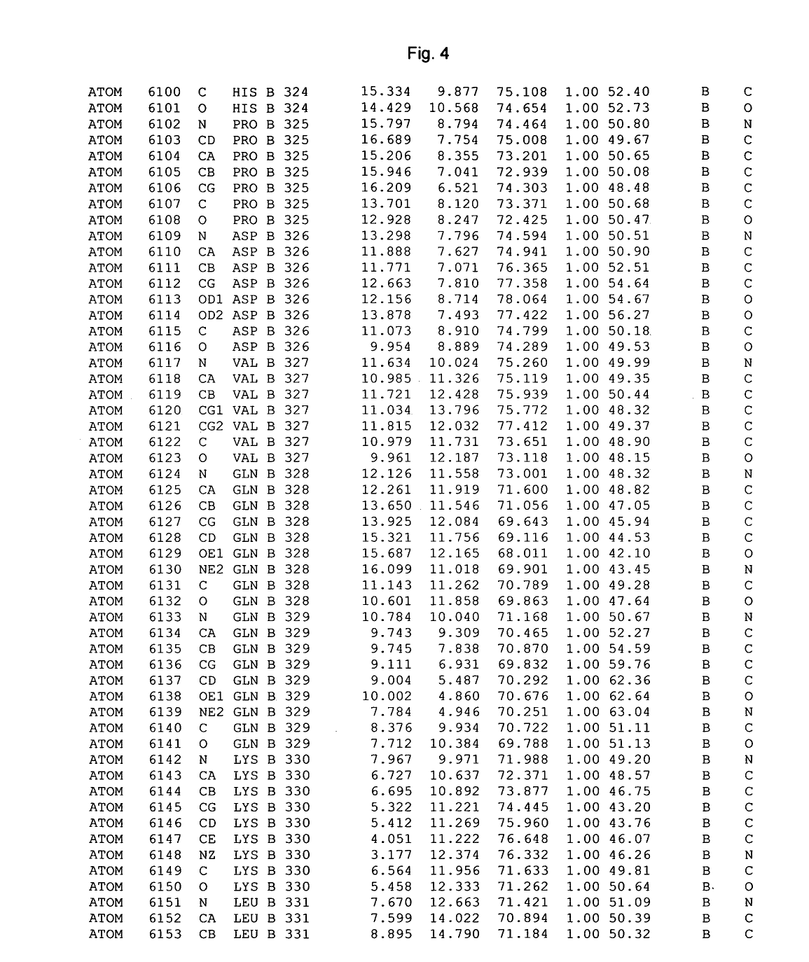

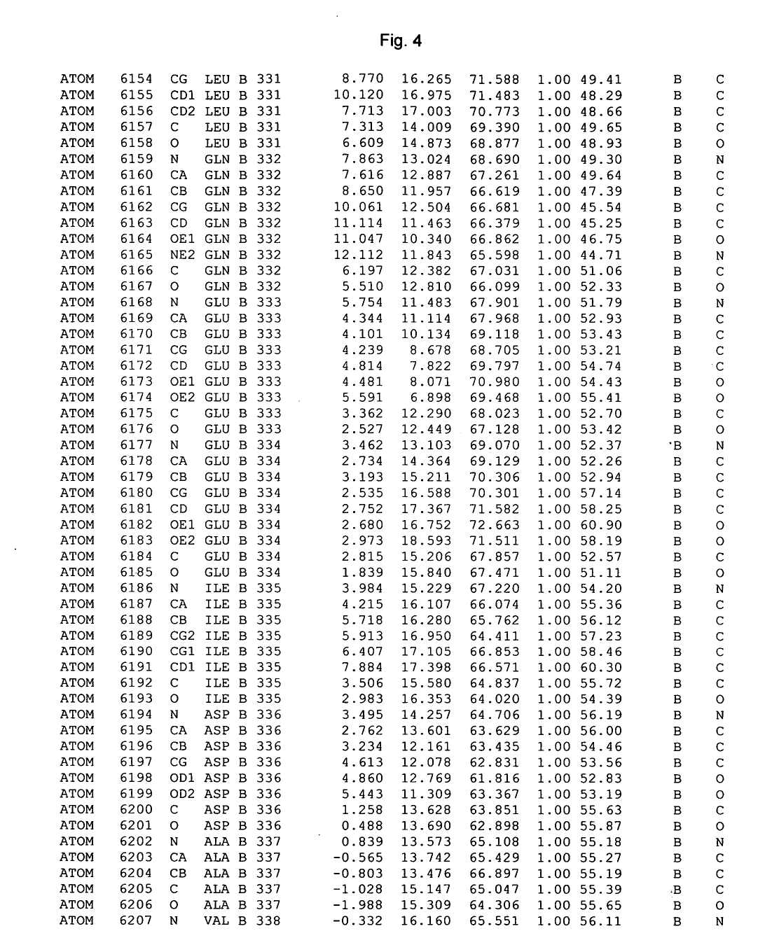

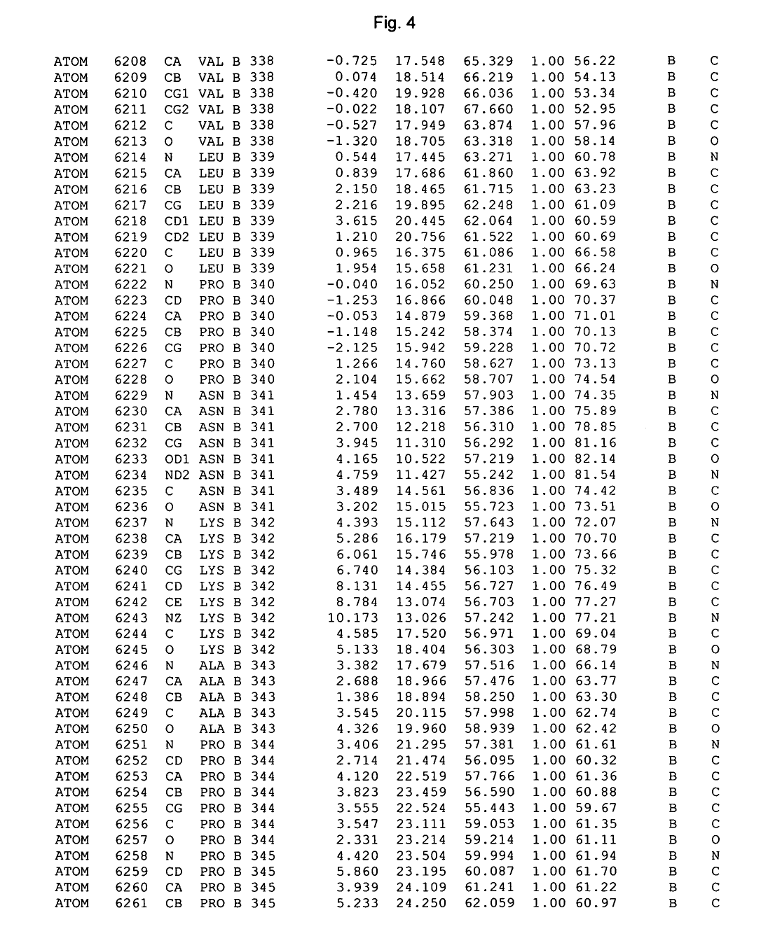

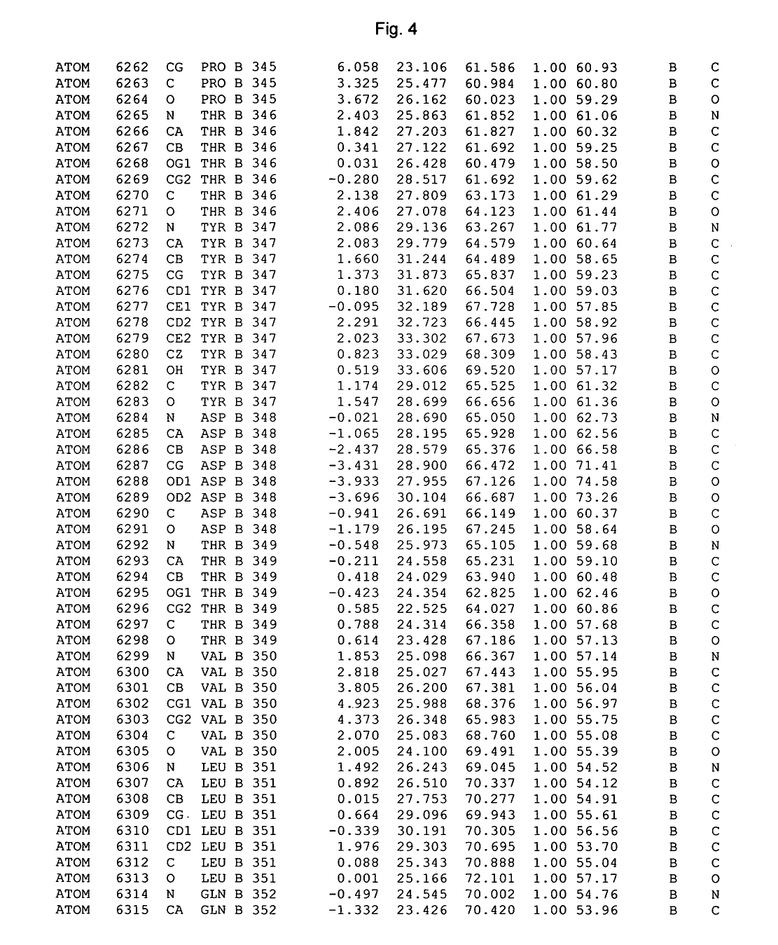

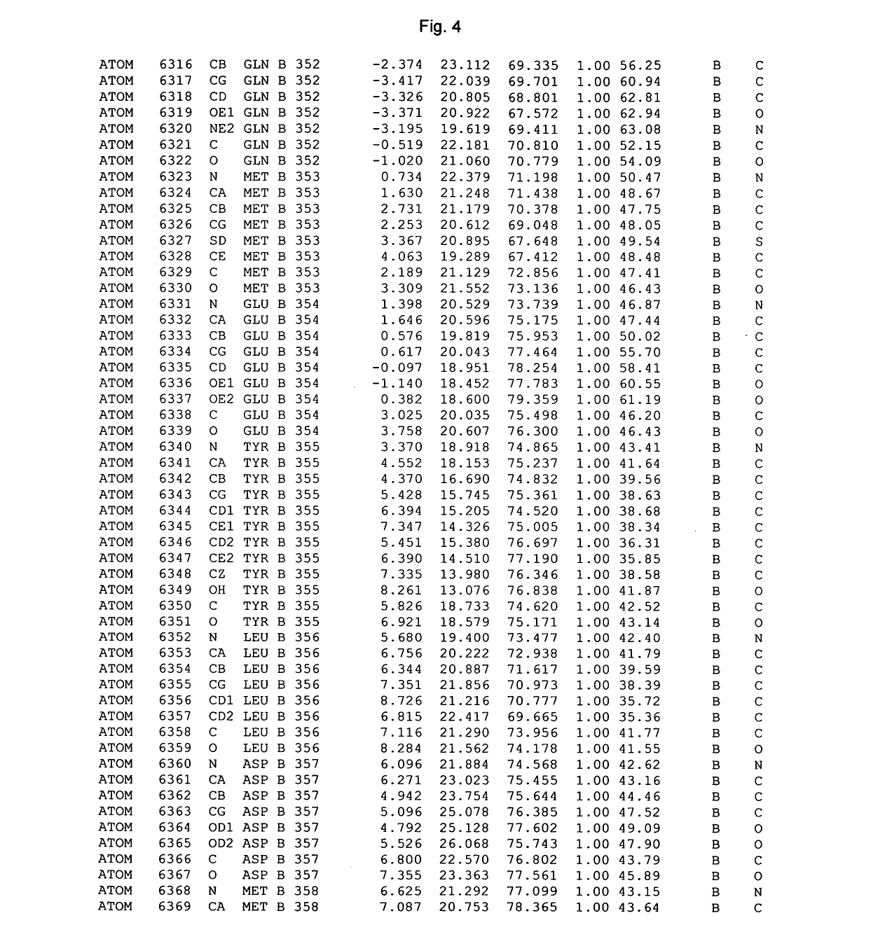

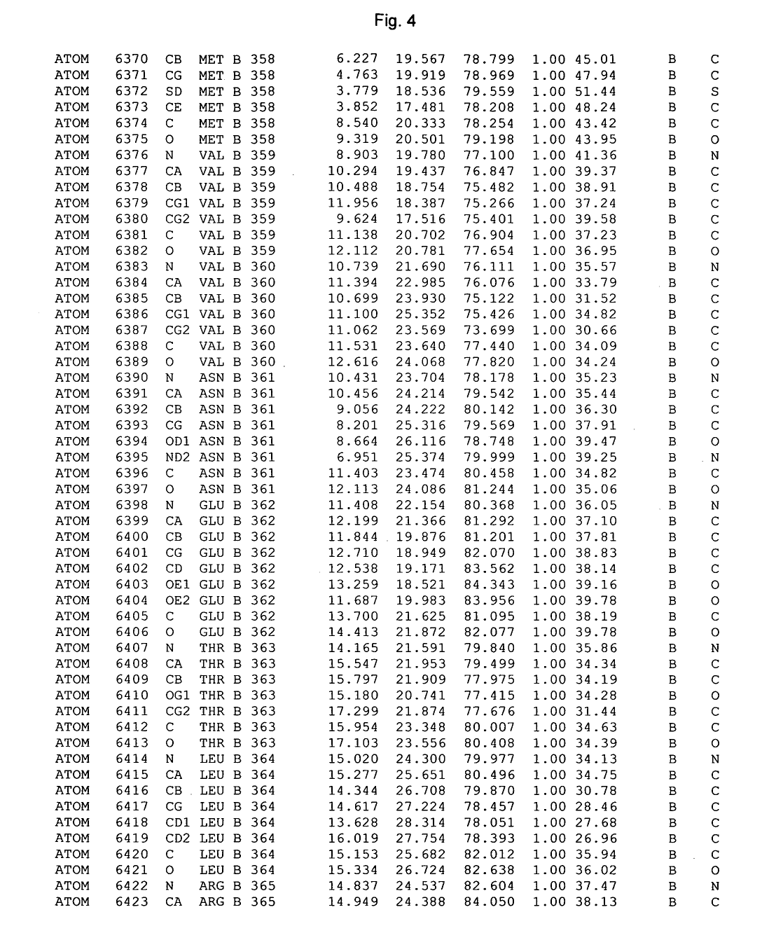

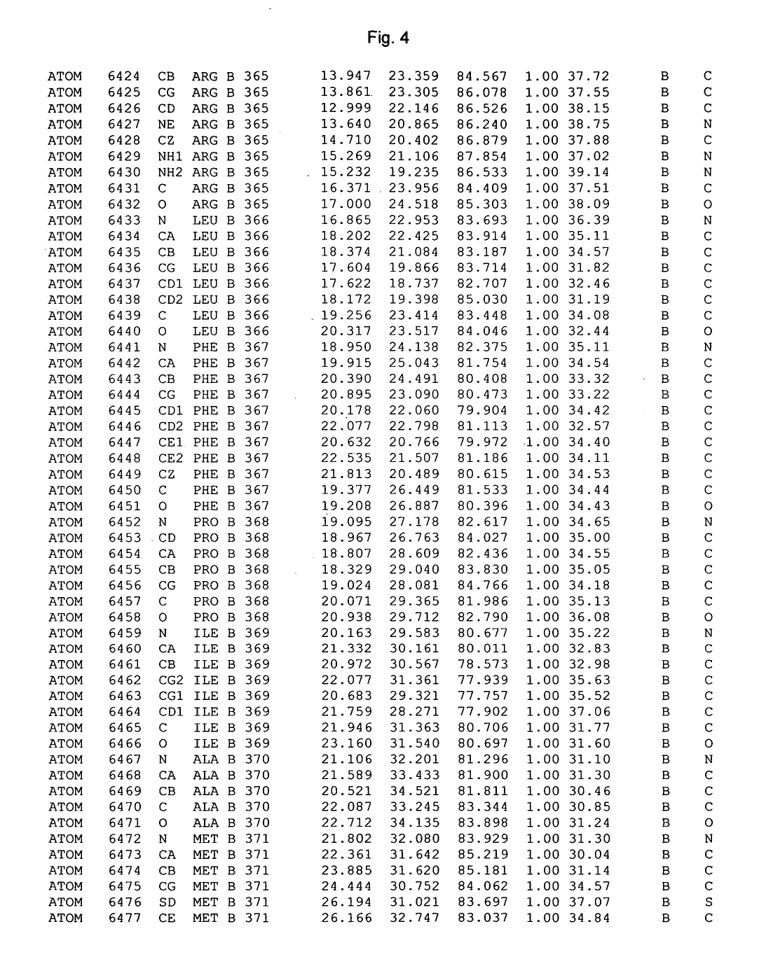

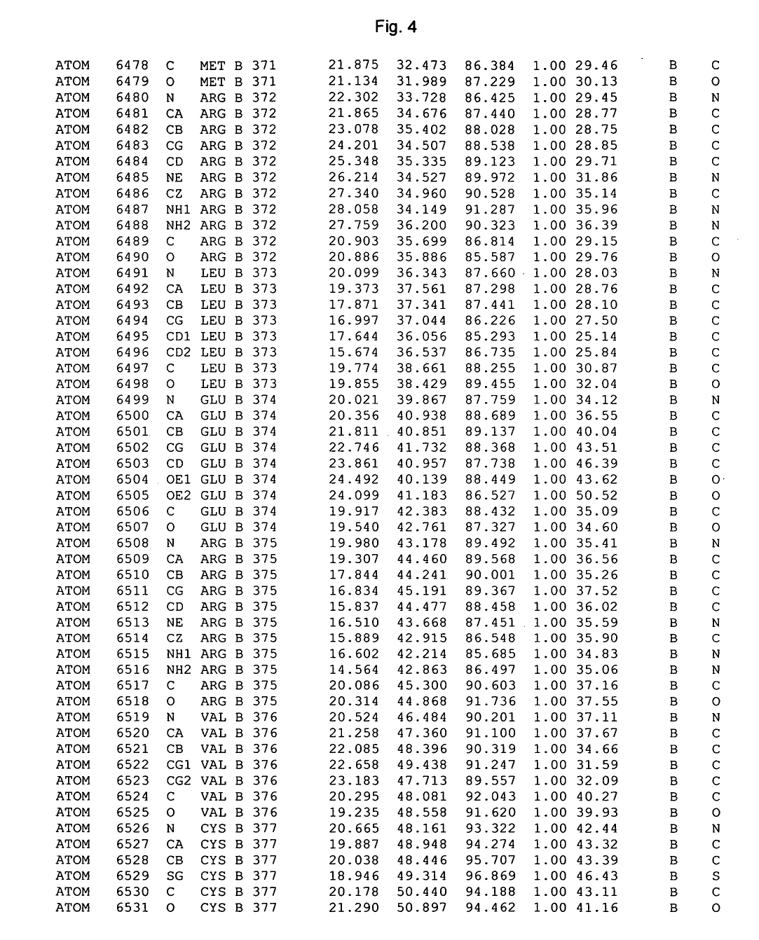

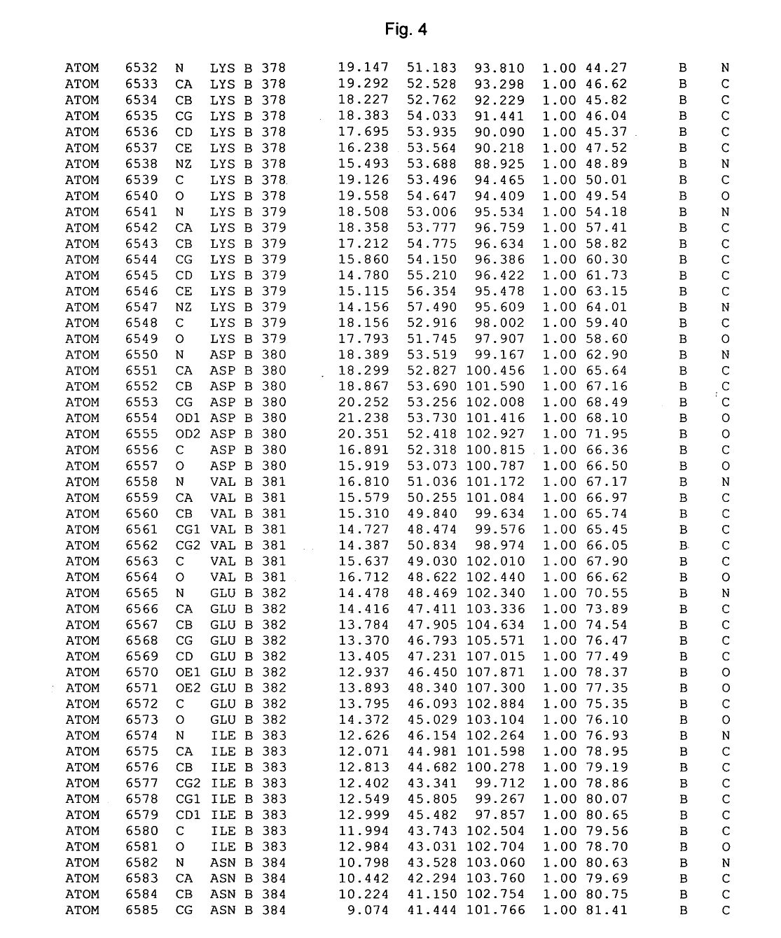

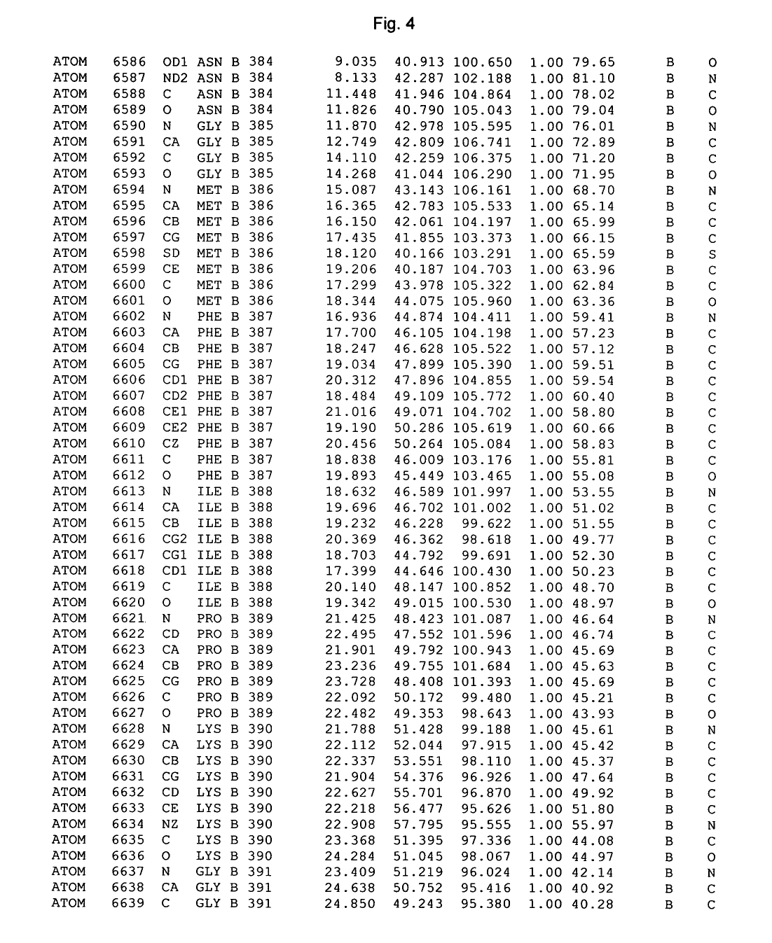

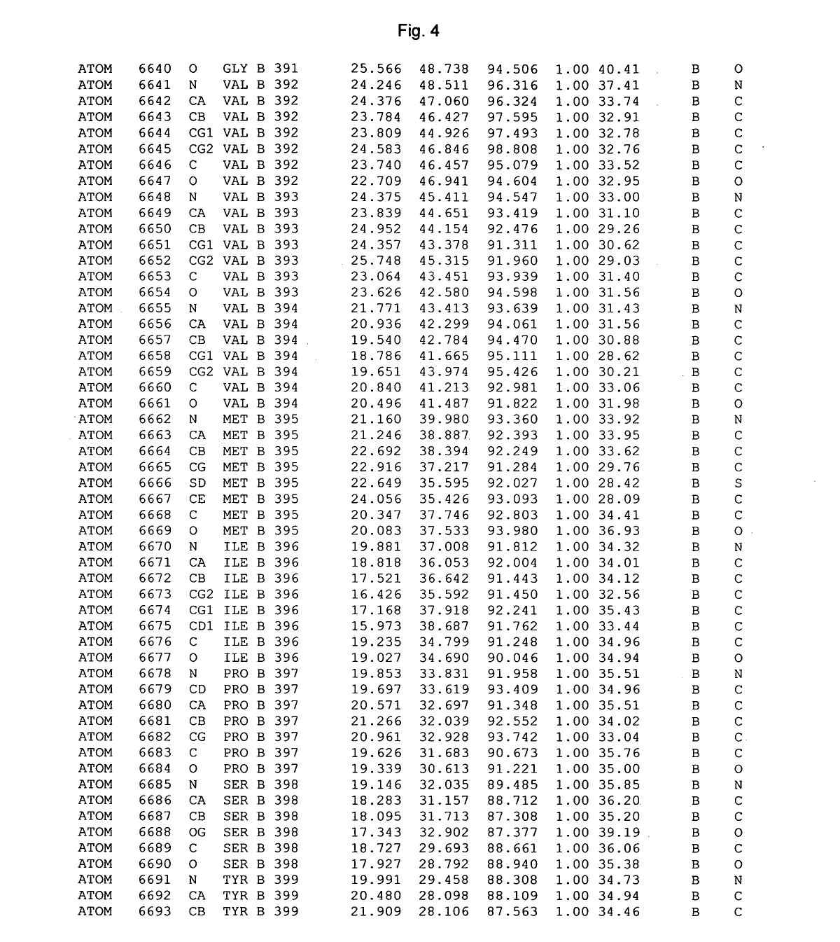

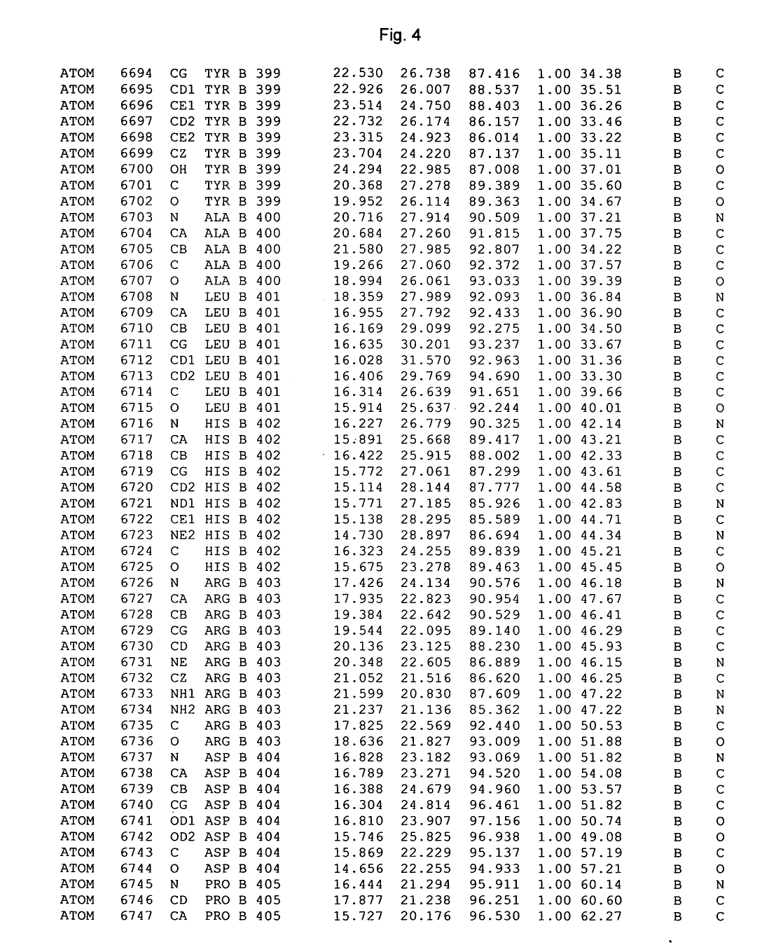

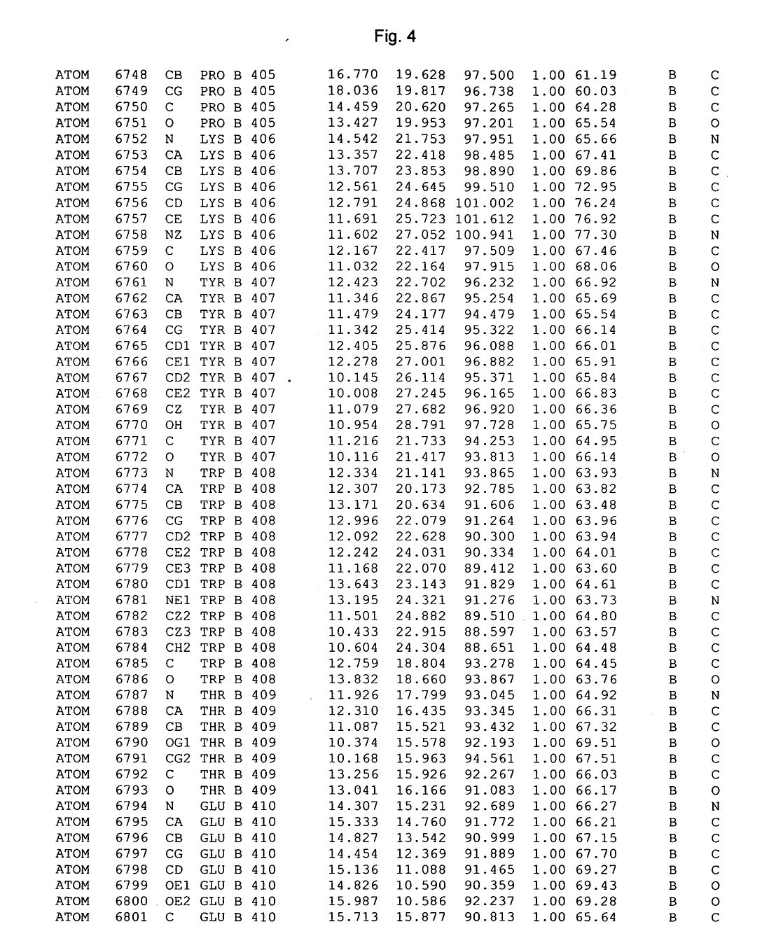

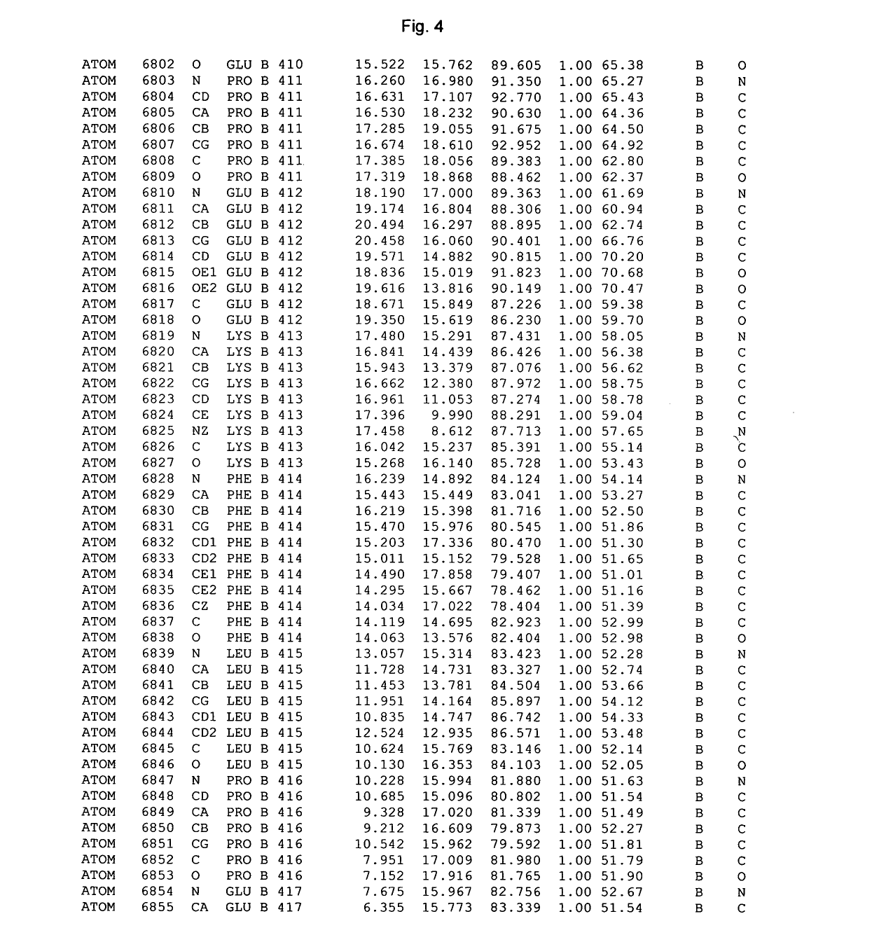

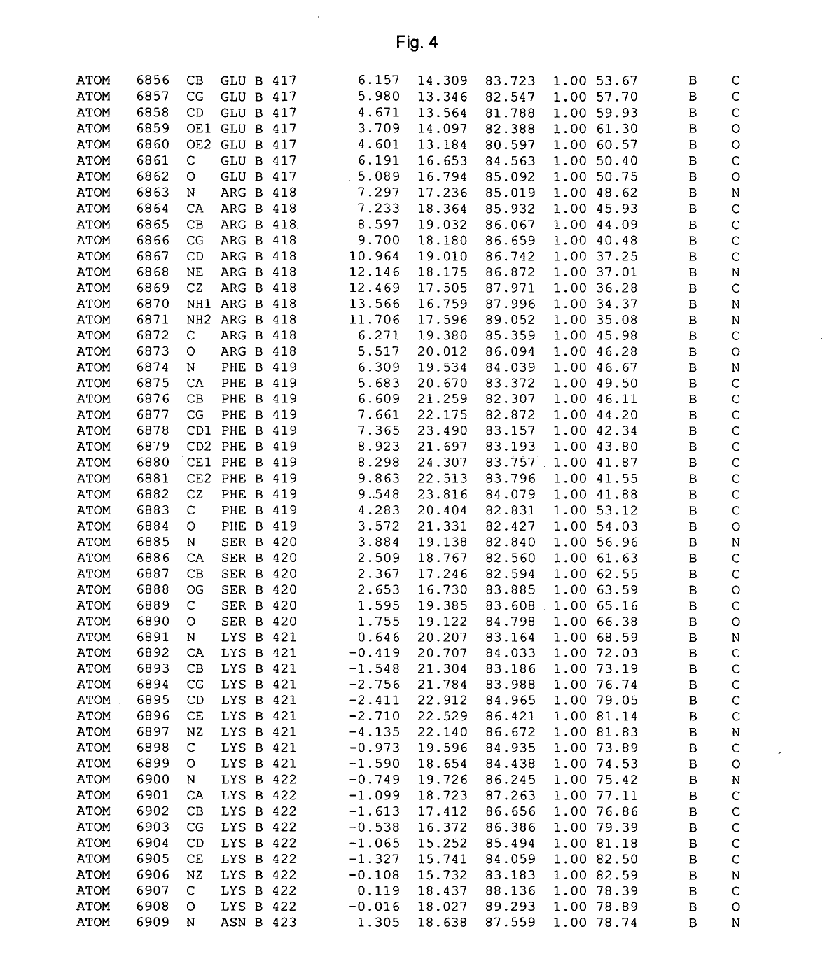

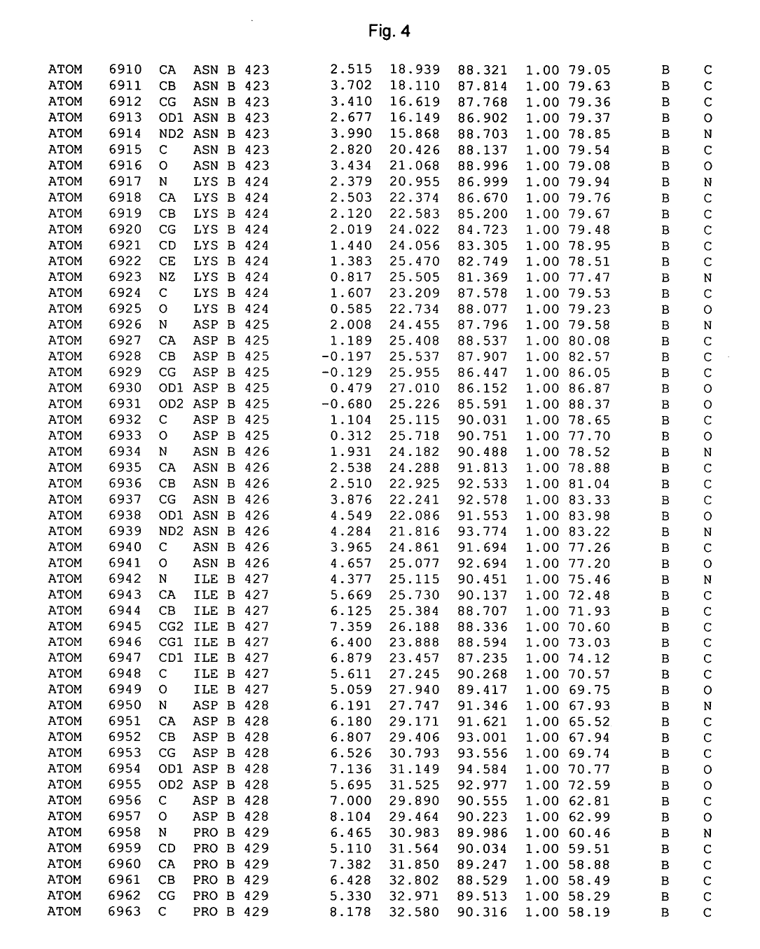

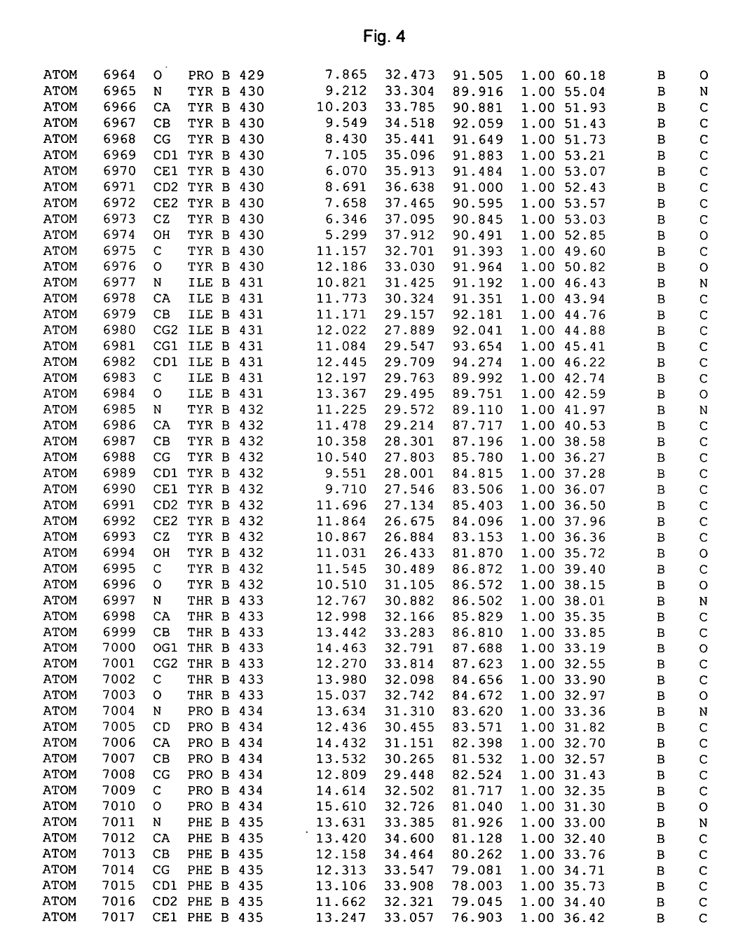

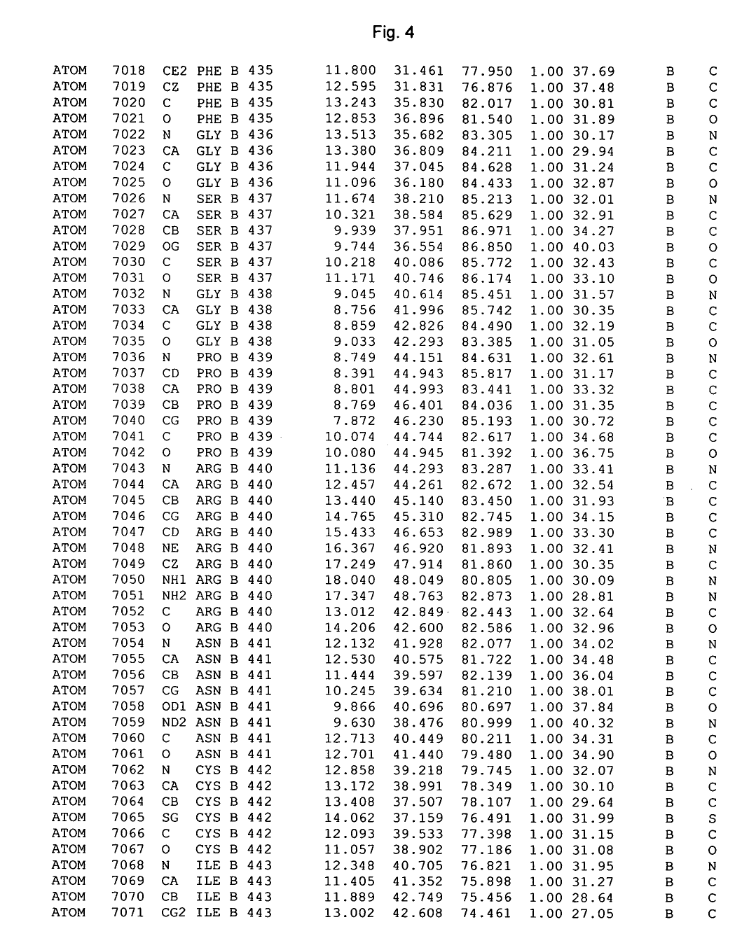

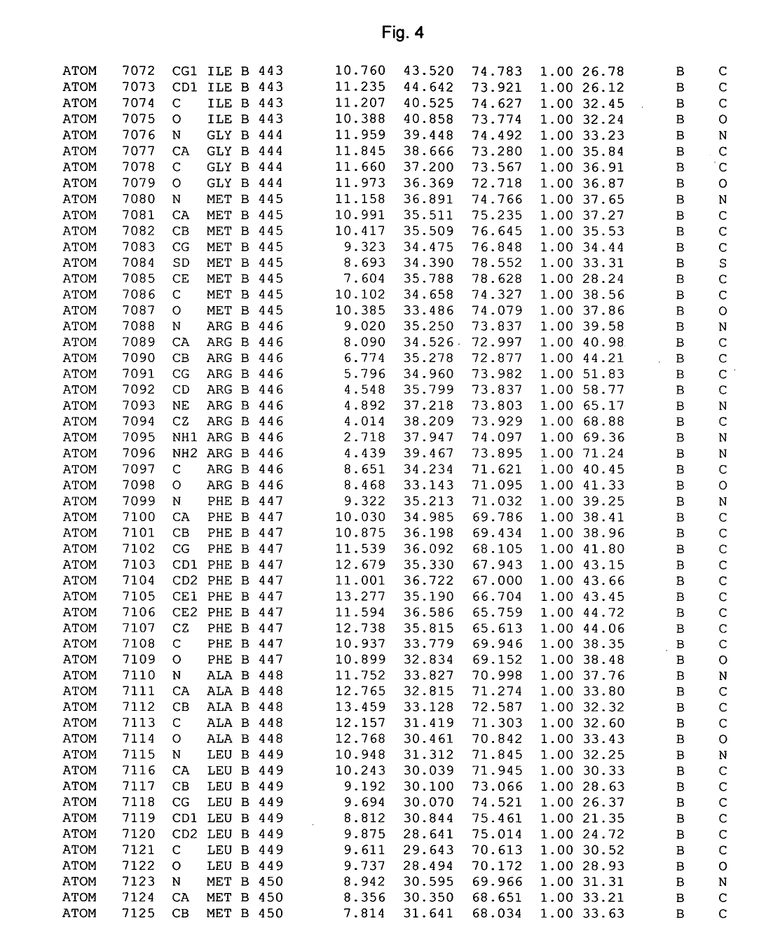

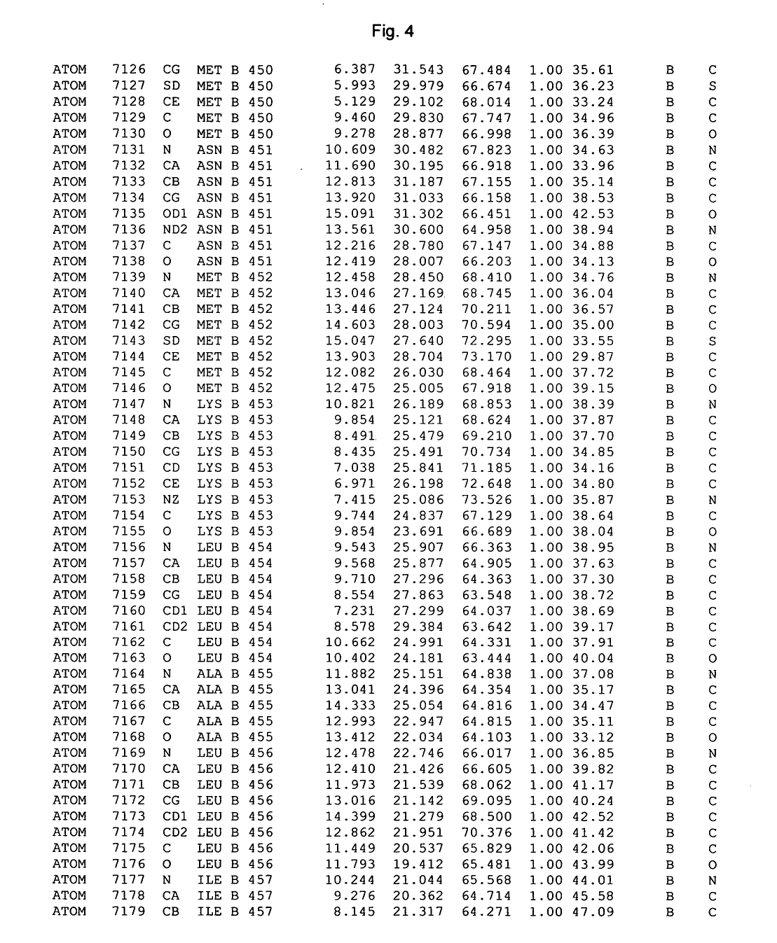

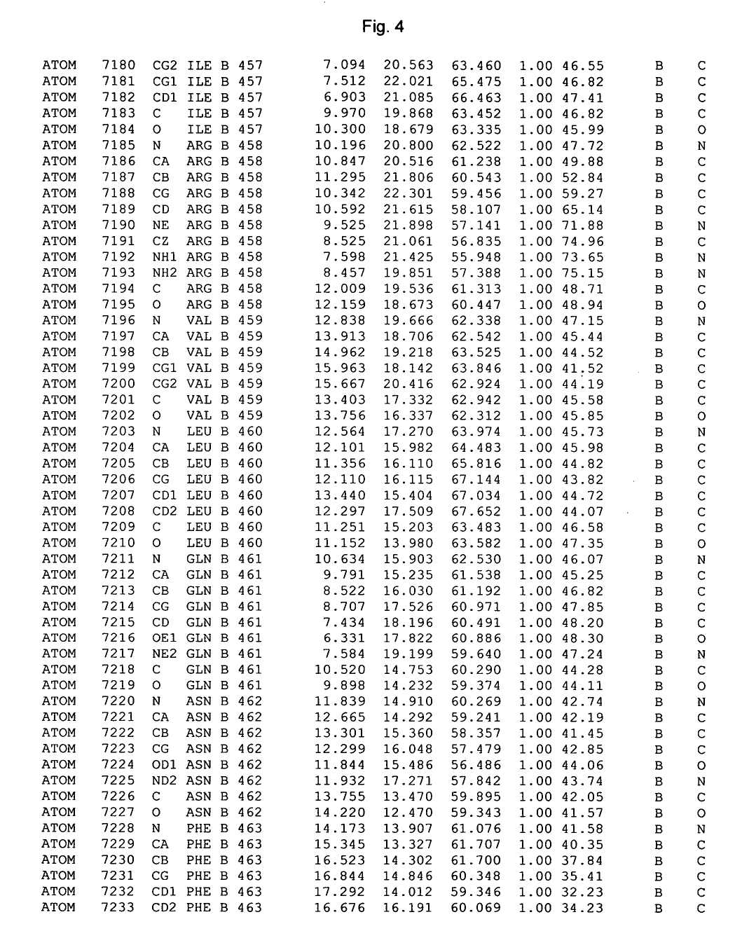

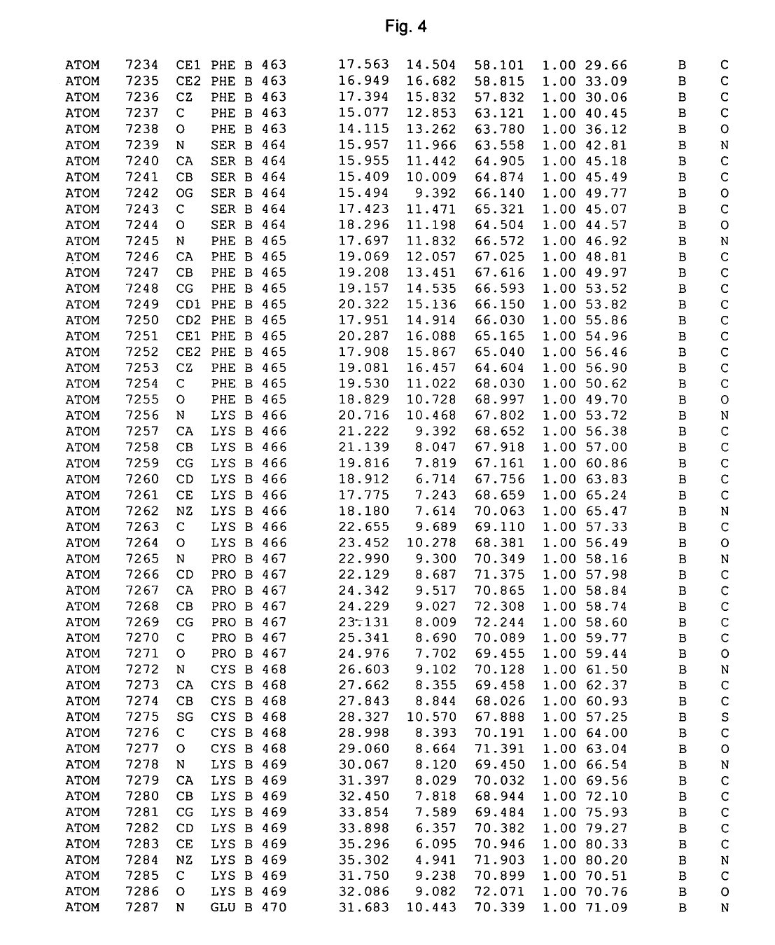

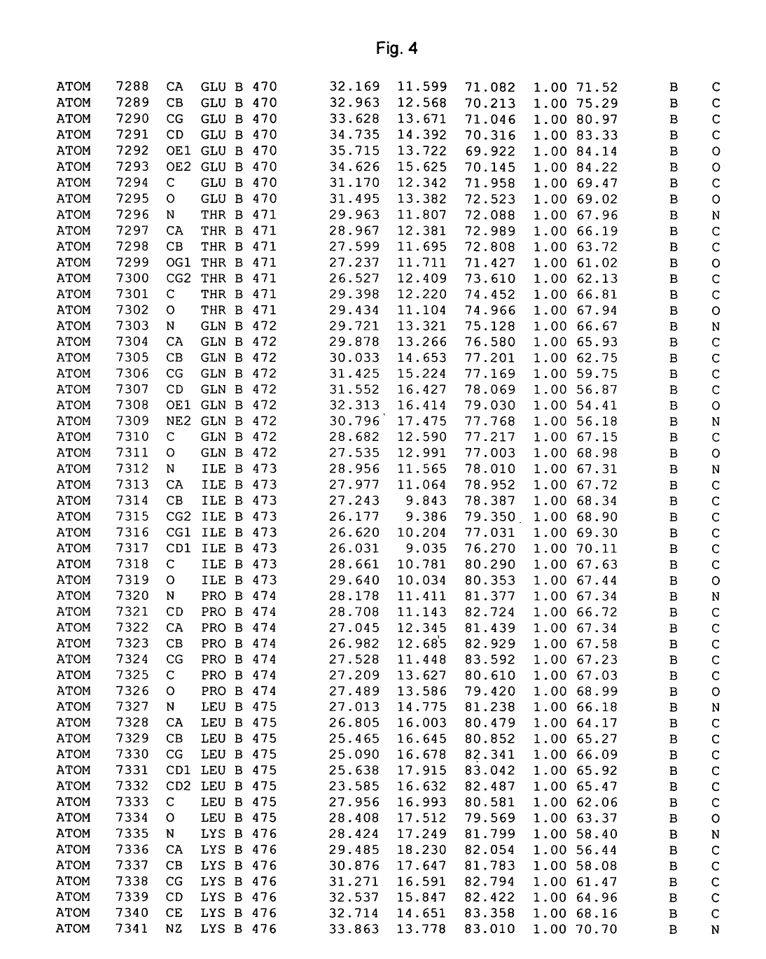

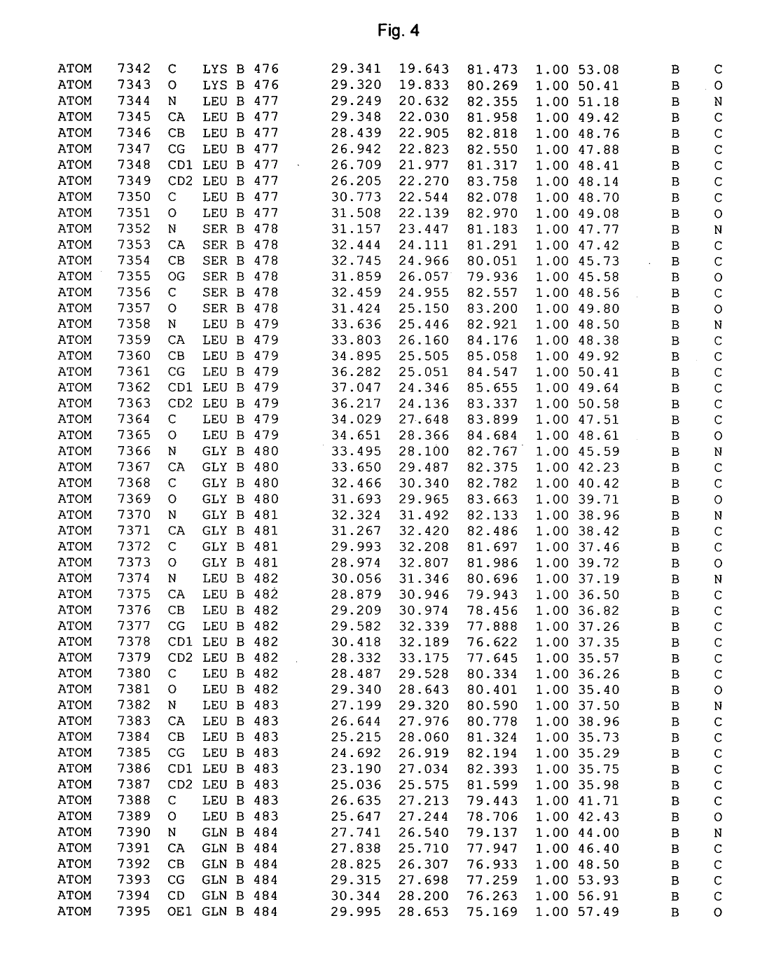

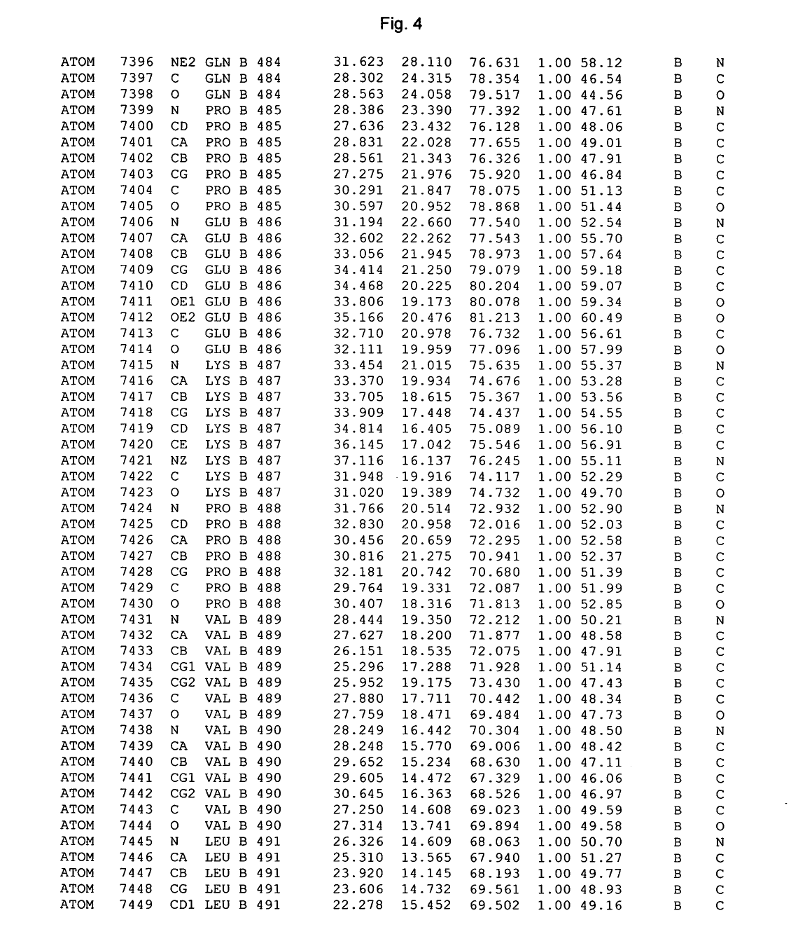

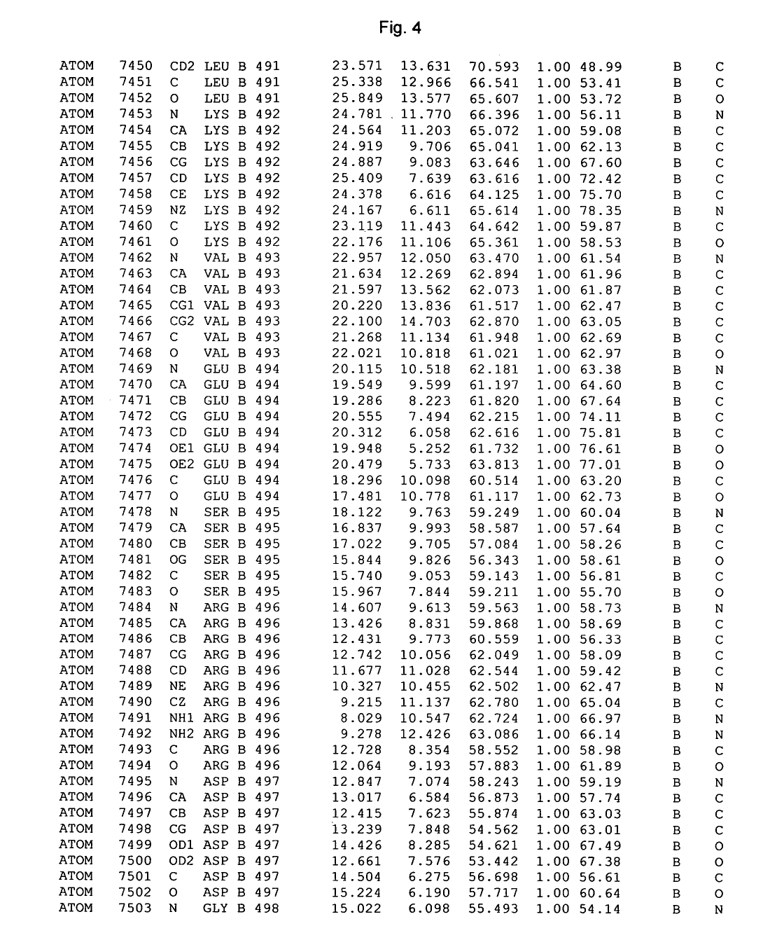

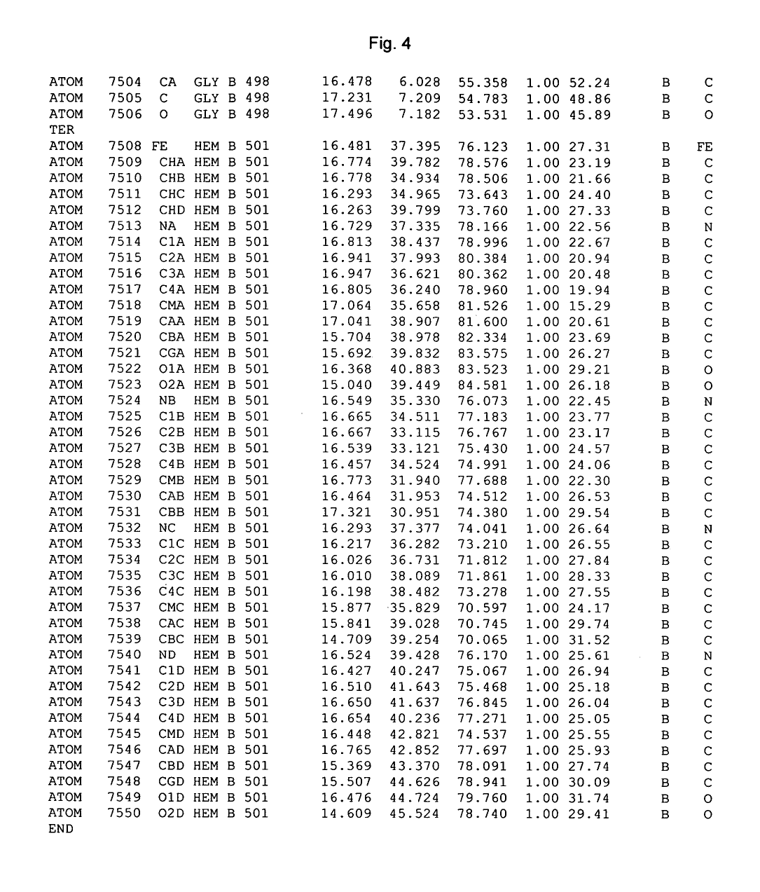

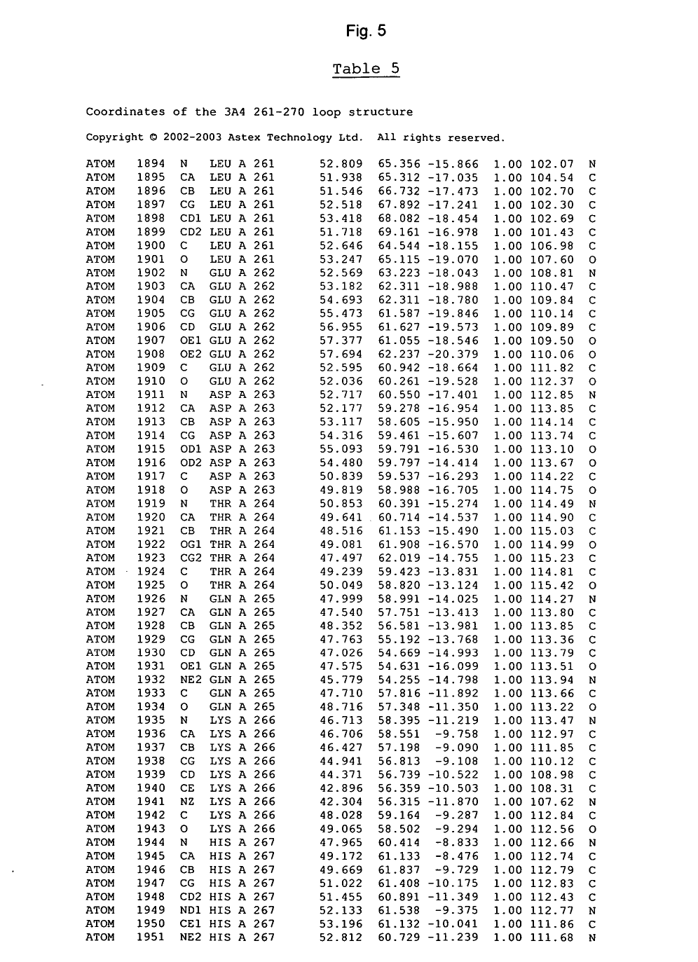

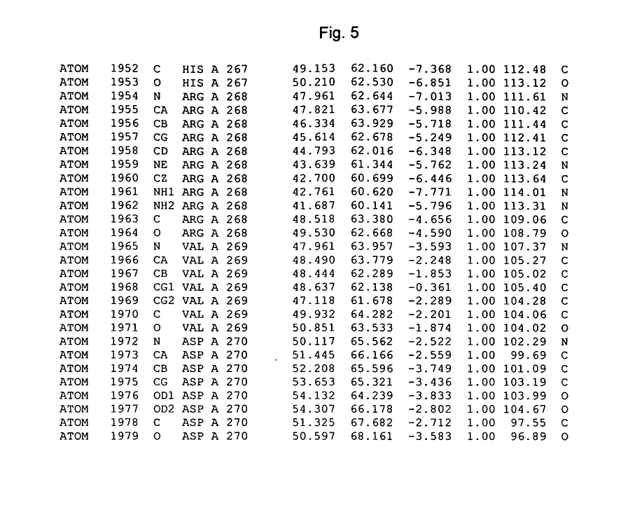

### DETAILED DESCRIPTION OF THE INVENTION

A. Protein Crystals.

The present invention provides a crystal of 3A4 having an orthorhomobic space group I222, and unit cell dimensions 78 Å, 100 Å, 132 Å, 90°, 90°, 90°. Unit cell variability of 5% may be observed in all dimensions. An example of such variability is a crystal of unit cell dimensions 77.94 Å, 100.91 Å, 131.00 Å.

In another aspect, the invention provides a crystal of 3A4 having a space group P2₁2₁2 with cell dimensions of about 88 Å, 111 Å, 113 Å, 90°, 90°, 90°. Unit cell variability of 5% may be observed in all dimensions.

In a further aspect, the invention provides a co-crystal of a 3A4 and a ligand, such as a compound selected from the group of metyrapone and progesterone.

Such crystals may be obtained using the methods described in the accompanying examples.

The crystal may be of a 3A4 protein which is desirably truncated in its N-terminal region to delete the hydrophobic trans-membrane domain, and the region is replaced by a short (e.g. 8 to 20) amino acid sequence. For expression of the human 3A4 P450, we have used an N-terminal sequence MAYGTHSHGLFKKLGI (SEQ ID NO:3) in place of the native N-terminal residues, which increases expression of the proteins in E. coli and increases solubility.

The 3A4 P450 may optionally comprise a tag, such as a C-terminal polyhistidine tag to allow for recovery and purification of the protein.

Our experiments have been based on the use of the particular N-terminal truncation mentioned above, and this protein also comprises a polyhistidine tag at the C-terminus. The N-terminal truncation and tag are both features which can be varied by those of skill in the art using routine skill. For example, alternative N-terminal sequence might be utilised, for example for production in host cells other than E. coli. Likewise, other tags may be used for purification of the protein as described below. These N- and C-terminal modification may be made to a 3A4 protein which retains the core sequence of the wild type protein from the residue 17 onwards of SEQ ID NO:2 shown herein, up to the residue immediately preceding the polyhistidine tag.

Where present, the N-terminal sequence is preferably not the full length wild-type sequence, and preferably smaller than 30, e.g. 20 residues in size. Preferably, it is shorter that the wild type sequence. Preferably, the N-terminal region is the truncation illustrated in the accompanying examples. This type of N-terminal sequence reduces the tendency of 3A4 to anchor to membranes and to aggregate compared to the wild type sequence. The truncation utilised here has wild-type residues 3-24 deleted.

Where present, the C-terminal sequence is preferably no larger than 30, and preferably no larger than 10 amino acids in size.

The 3A4 sequence may be that of the core sequence illustrated herein, or an allele thereof, or a variant which retains the ability to form crystals under the conditions illustrated herein. Such variants include those with a number of amino acid substitutions, for example 1, 2, 3, 4, 5, 6, 7, 8, 9 or 10 amino acids by an equivalent or fewer number of amino acids. Further examples of variants, including mutants, are discussed further herein below.

The methodology used to provide a P450 crystal illustrated herein may be used generally to provide a human 3A4 crystal resolvable at a resolution of at least 3.0 Å, and preferably at least 2.8 Å.

The invention thus further provides a 3A4 crystal having a resolution of at least 3.0 Å, preferably at least 2.8 Å.

The proteins may be wild-type proteins or variants thereof, which are modified to promote crystal formation, for example by N-terminal truncations and/or deletion of loop regions, which prevent crystal formation.

In a further aspect, the invention provides a method for making a P450 protein crystal, particularly of a 3A4 protein comprising the core sequence of 3A4 (as defined above) or a variant thereof, which method comprises growing a crystal by vapor diffusion using a reservoir buffer that contains 0.05-0.2 M HEPES pH 7.0-7.8, 2.5-10% IPA, 0-20% PEG 4000, 0-0.3 M sodium chloride, 0-10% PEG 400, 0-10% glycerol, preferably 0.1 M HEPES pH 7.2, 5% IPA, 10% PEG 4000. The crystal is grown by vapor diffusion and is performed by placing an aliquot of the solution on a cover slip as a hanging drop above a well containing the reservoir buffer. The concentration of the protein solution used was 0.3-0.7 mM.

Crystals of the invention also include crystals of 3A4 mutants, chimeras, homologues in the 3A family (e.g. 3A1, 3A5, 3A7, 3A12 and 3A43) and alleles.

(i) Mutants

A mutant is a 3A4 protein characterized by the replacement or deletion of at least one amino acid from the wild type 3A4. Such a mutant may be prepared for example by site-specific mutagenesis, or incorporation of natural or unnatural amino acids.

The present invention contemplates “mutants” wherein a “mutant” refers to a polypeptide which is obtained by replacing at least one amino acid residue in a native or synthetic 3A4 with a different amino acid residue and/or by adding and/or deleting amino acid residues within the native polypeptide or at the N- and/or C-terminus of a polypeptide corresponding to 3A4, and which has substantially the same three-dimensional structure as 3A4 from which it is derived. By having substantially the same three-dimensional structure is meant having a set of atomic structure co-ordinates that have a root mean square deviation (r.m.s.d.) of less than or equal to about 2.0 Å (preferably less than 1.55 or 1.5 Å, more preferably less than 1.0 Å, and most preferably less than 0.5 Å) when superimposed with the atomic structure co-ordinates of the 3A4 from which the mutant is derived when at least about 50% to 100% of the C_{α} atoms of the 3A4 are included in the superposition. A mutant may have, but need not have, enzymatic or catalytic activity.

To produce homologues or mutants, amino acids present in the said protein can be replaced by other amino acids having similar properties, for example hydrophobicity, hydrophobic moment, antigenicity, propensity to form or break α-helical or β-sheet structures, and so on. Substitutional variants of a protein are those in which at least one amino acid in the protein sequence has been removed and a different residue inserted in its place. Amino acid substitutions are typically of single residues but may be clustered depending on functional constraints e.g. at a crystal contact. Preferably amino acid substitutions will comprise conservative amino acid substitutions. Insertional amino acid variants are those in which one or more amino acids are introduced. This can be amino-terminal and/or carboxy-terminal fusion as well as intrasequence. Examples of amino-terminal and/or carboxy-terminal fusions are affinity tags, MBP tag, and epitope tags.

Amino acid substitutions, deletions and additions which do not significantly interfere with the three-dimensional structure of the 3A4 will depend, in part, on the region of the 3A4 where the substitution, addition or deletion occurs. In highly variable regions of the molecule, non-conservative substitutions as well as conservative substitutions may be tolerated without significantly disrupting the three-dimensional structure of the molecule. In highly conserved regions, or regions containing significant secondary structure, conservative amino acid substitutions are preferred.

Conservative amino acid substitutions are well-known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

In some instances, it may be particularly advantageous or convenient to substitute, delete and/or add amino acid residues in order to provide convenient cloning sites in the cDNA encoding the polypeptide, to aid in purification of the polypeptide, etc. Such substitutions, deletions and/or additions which do not substantially alter the three dimensional structure of 3A4 will be apparent to those having skills in the art.

It should be noted that the mutants contemplated herein need not exhibit enzymatic activity. Indeed, amino acid substitutions, additions or deletions that interfere with the catalytic activity of the 3A4 but which do not significantly alter the three-dimensional structure of the catalytic region are specifically contemplated by the invention. Such crystalline polypeptides, or the atomic structure co-ordinates obtained there from, can be used to identify compounds that bind to the protein.

The residues for mutation could easily be identified by those skilled in the art and these mutations can be introduced by site-directed mutagenesis e.g. using a Stratagene QuikChange™ Site-Directed Mutagenesis Kit or cassette mutagenesis methods (see e.g. Ausubel et al., eds., Current Protocols in Molecular Biology, John Wiley & Sons, Inc., New York, and Sambrook et al., Molecular Cloning: a Laboratory Manual, 2nd ed., Cold Spring Harbor Laboratory Press, Cold Spring Harbor, N.Y., (1989)).

(ii) Alleles

The present invention contemplates “alleles” wherein allele is a term coined by Bateson and Saunders (1902) for characters which are alternative to one another in Mendelian inheritance (Gk. Allelon, one another; morphe, form). Now the term allele is used for two or more alternative forms of a gene resulting in different gene products and thus different phenotypes. An allele contains nucleotide changes that have been shown to affect transcription, splicing, translation, post-transcriptional or post-translational modifications or result in at least one amino acid change. These different alleles are particularly important in P450s as some confer different metabolic clearance rates of specific drugs onto the phenotype. Alleles of P450s are often only different by one or two amino acids. As of 2002, 25 alleles of 3A4 have been identified, where wild type is CYP3A4*1A (NCBI ACCESSION M18907, Gonzalez F J, Schmid B J, Umeno M, Mcbride O W, Hardwick J P, Meyer U A, Gelboin H V, Idle J R, DNA 1988 March;7(2):79-86).

To the extent that the present invention relates to 3A4-ligand complexes and mutant, homologue, analogue, allelic form, species variant proteins of 3A4, crystals of such proteins may be formed. The skilled person would recognize that the conditions provided herein for crystallising 3A4 may be used to form such crystals. Alternatively, the skilled person would use the conditions as a basis for identifying modified conditions for forming the crystals.

Thus the aspects of the invention relating to crystals of 3A4, may be extended to crystals of mutant and mutants of 3A4 which result in homologue, allelic form, and species variant.

(iii) Crystallization of 3A4

To produce crystals of 3A4 protein the final protein is, conveniently, concentrated to 10-60, e.g. 20-40 mg/ml in 10-100 mM potassium phosphate with high salt (e.g. 500 mM NaCl or KCl), optionally also with about 1 mM EDTA and/or about 2 mM dithiothreitol, by using concentration devices which are commercially available. Crystallisation of the protein is set up by the 0.5-2 μl hanging or sitting drop methods and the protein is crystallised by vapor diffusion at 5-25° C. against a range of vapor diffusion buffer compositions. It is customary to use a 1:1 ratio of protein solution and vapor diffusion buffer in the hanging drop, and this has been used herein unless stated to the contrary.

Typically the vapor diffusion buffer comprises 0-27.5%, preferably 2.5-27.5% PEG 1K-20 K, preferably 1-8K or PEG 2000MME-5000MME, preferably PEG 2000 MME, or 0-10% Jeffamine M-600 and/or 5-20%, e.g. 10-20% propanol or 15-20% ethanol or about 15%-30%, e.g. about 15% 2-methyl-2,4-pentanediol (MPD), optionally with 0.01 M-1.6 M salt or salts and/or 0-0.15, e.g. 0-0.1, M of a solution buffer and/or 0-35%, such as 0-15%, glycerol and/or 0-35% PEG300-400; but preferably:

10-25% PEG 1K-8K or PEG 2000MME or 0-10% Jeffamine M-600 and/or 5-15%, e.g. 10-15%, propanol or ethanol, optionally with 0.1 M-0.2 M salt or salts and/or 0-0.15, e.g. 0-0.1 M solution buffer and/or PEG400, but more preferably:

15-20% PEG 3350 or PEG 4000 or PEG 2000MME or 0-10% Jeffamine M-600 or 5-15%, e.g. 10-15% propanol or ethanol, optionally with 0.1 M-0.2 M salt or salts and/or 0-0.15 M solution buffer.

Alternatively the vapor diffusion buffer may be 0.1 M HEPES pH 7.5 0.2-0.3 M potassium chloride, 1-5% MPD, 7-14.0% PEG 3350 or PEG 4000, 25-50 mM calcium chloride more specifically 0.1 M HEPES pH 7.5, 0.20-0.30 M KCl, 10-14% PEG 4000, 5% MPD, 25 mM calcium chloride.

It has also been found that crystals of the space group P2₁2₁2 may be preferentially obtained using a vapor diffusion buffer of 0.1 Tris-Acetic acid pH 7.5-8.5, 0.8-1.0M sodium formate, 9.5-17.5% MPEG 2000 with 0-5% glycerol, or 0-5% PEG 400 or 0-5% ethylene glycol. More specifically such a buffer may comprise 0.1 Tris-acetic acid pH 7.5-8.5, 0.8-0.9M sodium formate, 10.5-17.5% MPEG 2000, particularly 0.1 Tris-Acetic acid pH 7.5, 0.9M Sodium formate, 10.5-12.5% MPEG 2000, or 0.1 Tris-acetic acid pH 8.5, 0.8M sodium formate, 17.5% MPEG 2000.

The salt may be an alkali metal (particularly lithium, sodium and potassium), alkaline earth metal (e.g. magnesium or calcium), ammonium, ferric, ferrous or transition metal salt (e.g. zinc) of a halide (e.g. bromide, chloride or fluoride), acetate, formate, nitrate, sulfate, tartrate, citrate or phosphate. This includes sodium fluoride, potassium fluoride, ammonium fluoride, ammonium acetate, lithium acetate, magnesium acetate, sodium acetate, potassium acetate, calcium acetate, zinc acetate, ammonium chloride, lithium chloride, magnesium chloride, potassium chloride, sodium chloride, potassium bromide, magnesium formate, sodium formate, potassium formate, ammonium formate, ammonium nitrate, lithium nitrate, potassium nitrate, sodium nitrate, ammonium sulfate, potassium sulfate, lithium sulfate, sodium sulfate, di-sodium tartrate, potassium sodium tartrate, di-ammonium tartrate, potassium dihydrogen phosphate, tri-sodium citrate, tri-potassium citrate, zinc acetate, ferric chloride, calcium chloride, magnesium nitrate, magnesium sulfate, sodium dihydrogen phosphate, di-sodium hydrogen phosphate, di-potassium hydrogen phosphate, ammonium dihydrogen phosphate, di-ammonium hydrogen phosphate, tri-lithium citrate, nickel chloride, ammonium iodide, di-ammonium hydrogen citrate.

Solution buffers if present include, for example, Hepes, Tris, imidazole, cacodylate, tri-sodium citrate/citric acid, tri-sodium citrate/HCl, acetic acid/sodium acetate, phosphate-citrate, sodium potassium phosphate, 2-(N-morpholino)-ethane sulphonic acid/NaOH (MES), CHES or bis-trispropane.

The pH range is desirably maintained at pH 4.2-8.5, preferably 4.7-8.5.

Solution buffers if present can also include, for example, bicine, bis-tris, CAPS, MOPS, ADA which allow the pH to be maintained in the range 5.8-11.

Crystals may be prepared using a Hampton Research Screening kits, Poly-ethylene glycol (PEG)/ion screens, PEG grid, Ammonium sulphate grid, PEG/ammonium sulphate grid or the like.

Crystallisation may also be performed in the presence of an inhibitor of P450, e.g. fluvoxamine or 2-phenyl imidazole. 3A4 crystallisation may also be performed in the presence of one or more inhibitors e.g. ketoconazole, metyrapone, fluconazole or triadimefon and/or in the presence of one or more substrate(s) e.g. testosterone or progesterone.

Additives can be added to a crystallisation condition identified to influence crystallisation. Additive Screens are to be used during the optimisation of preliminary crystallisation conditions where the presence of additives may assist in the crystallisation of the sample and the additives may improve the quality of the crystal e.g. Hampton Research additive screens which use glycerol, polyols and other protein stabilizing agents in protein crystallisation (R. Sousa. Acta. Cryst. (1995) D51, 271-277) or divalent cations (Trakhanov, S. and Quiocho, F. A. Protein Science (1995) 4,9, 1914-1919).

In addition, detergents may be added to a crystallisation condition to improve the crystallisation behaviour e.g. the ionic, non-ionic and zwitterionic detergents found in the Hampton Research detergent screens (McPherson, A., et al., The effects of neutral detergents on the crystallization of soluble proteins, J. Crystal Growth (1986) 76, 547-553).

Alternatively, the vapor diffusion buffer typically comprises 0-27.5% PEG 1K-20 K, preferably 1-8K or PEG 2000MME-5000MME, preferably PEG 2000 MME, or 0-10% Jeffamine M-600 and/or 1-20%, e.g. 1-20% propanol or 15-20% ethanol or about 1%-30%, e.g. about 2-25% 2-methyl-2,4-pentanediol (MPD), optionally with 0.01 M-1.6 M salt or salts and/or 0-0.15 M, e.g. 0-0.1 M, of a solution buffer and/or 0-35%, such as 0-15%, glycerol and/or 0-35% PEG300-400; but preferably:

0-27.5%, preferably 2.5-27.5% PEG 1K-20 K, most preferably 5-20% PEG 4K or PEG 2000MME-5000MME, preferably PEG 2000 MME, and 1-20% alcohol, e.g. 1-20% propanol e.g. iso-propanol or 2-25% 2-methyl-2,4-pentanediol (MPD), optionally with 0.01 M-1.6 M salt or salts and/or 0-0.15 M, e.g. 0-0.1 M, of a solution buffer and/or 0-35%, such as 0-15%, glycerol and/or 0-35% PEG300-400.

B. Crystal Coordinates.

In a further aspect, the invention also provides a crystal of P450 having the three dimensional atomic coordinates of any one of Tables 1-4. The atomic coordinates of Tables 1-4 exclude most of the residues from a loop region (261-270), which are not as clear and amenable for unambiguous interpretation as other regions of the protein. It is possible that this loop may adopt a different conformation under different conditions e.g. data from a different crystal, upon additional of compound, and the like. Crystals of the invention will thus comprise the coordinates of any one of Tables 1-4, with the coordinates of the loop region optionally being as further described herein, though other atomic coordinates for this loop region are not excluded.

An advantageous feature of the structures defined by the atomic coordinates of Tables 1-4 are that they have a resolution of about 2.8 Å. More particularly, the residues in the binding pocket, and in the case of Tables 2 and 3, ligands in the binding pocket, are well resolved.

A further advantage of the 3A4 structure of Table 1 described herein is that it is an unliganded, apo structure. This makes it particularly suitable for soaking in ligands and hence determining co-complex structures and is also ideal for homology modelling purposes as there is no conformational bias from a ligand.

Tables 1-4 give atomic coordinate data for P450 3A4. In these Tables the third column denotes the atom, the fourth the residue type, the fifth the chain identification, the sixth the residue number (the atom numbering is with respect to the full length wild type protein), the seventh, eighth and ninth columns are the X, Y, Z coordinates respectively of the atom in question, the tenth column the occupancy of the atom, the eleventh the temperature factor of the atom, the twelfth the atom type.

Tables 1-4 are set out in an internally consistent format. For example (except in the case of Tyr 25), the coordinates of the atoms of each amino acid residue are listed such that the backbone nitrogen atom is first, followed by the C-alpha backbone carbon atom, designated CA, followed by side chain residues (designated according to one standard convention) and finally the carbon and oxygen of the protein backbone. Alternative file formats (e.g. such as a format consistent with that of the EBI Macromolecular Structure Database (Hinxton, UK)) which may include a different ordering of these atoms, or a different designation of the side-chain residues, ligand or haem molecule atoms, may be used or preferred by others of skill in the art. However it will be apparent that the use of a different file format to present or manipulate the coordinates of the Table is within the scope of the present invention.

The coordinates of Tables 1-4 provide a measure of atomic location in Angstroms, given to 3 decimal places. The coordinates are a relative set of positions that define a shape in three dimensions, but the skilled person would understand that an entirely different set of coordinates having a different origin and/or axes could define a similar or identical shape. Furthermore, the skilled person would understand that varying the relative atomic positions of the atoms of the structure so that the root mean square deviation of the residue backbone atoms (i.e. the nitrogen-carbon-carbon backbone atoms of the protein amino acid residues) is less than 2.0 Å, preferably less than 1.55 or 1.5 Å, more preferably less than 1.0 Å, more preferably less than 0.5 Å, more preferably less than 0.3 Å, such as less than 0.25 Å, or less than 0.2 Å, and most preferably less than 0.1 Å, when superimposed on the coordinates provided in Tables 1-4 for the residue backbone atoms, will generally result in a structure which is substantially the same as the structure of Tables 1-4 in terms of both its structural characteristics and usefulness for structure-based analysis of P450-interactivity molecular structures.

Likewise the skilled person would understand that changing the number and/or positions of the water molecules molecules of the Tables will not generally affect the usefulness of the structures for structure-based analysis of P450-interacting structure. Thus for the purposes described herein as being aspects of the present invention, it is within the scope of the invention if: the coordinates of any of Tables 1-4 are transposed to a different origin and/or axes; the relative atomic positions of the atoms of the structure are varied so that the root mean square deviation of residue backbone atoms is less than 2.0 Å, preferably less than 1.55 or 1.5 Å, more preferably less than 1.0 Å, and most preferably less than 0.5 Å when superimposed on the coordinates provided in any of Tables 1-4 for the residue backbone atoms; and/or the number and/or positions of water molecules is varied.

Furthermore, in the case of Tables 2 and 3, the coordinate data of metyrapone and progesterone respectively may be discarded by those of skill in the art seeking to utilise the 3A4 protein structures of these Tables. It thus will be generally understood that reference to the use of the coordinate data of Tables 2 and 3 refers to the use of the 3A4 protein coordinate data of said Tables.

Reference herein to the coordinate data of Tables 1-4 and the like thus includes the coordinate data in which one or more individual values of the Table are varied in this way. By “root mean square deviation” we mean the square root of the arithmetic mean of the squares of the deviations from the mean.

With regard to the loop region referred to above, comparision of the different P450 structures determined to date indicates that various loops within the proteins can adopt very different conformations, often in response to compound binding. In the apo and co-crystal forms of 3A4 which have been crystallized herein, a possible form of the loop region 261-270 is set out in Table 5. Thus in one aspect, the invention provides a crystal or crystal structure of P450 comprising amino acids having the atomic coordinates of any one of Tables 1-4, wherein the crystal additionally comprises amino acids having the atomic coordinates of Table 5.

Unless explicitly set out to the contrary, or otherwise clear from the context, reference throughout the present specification to the use of all or selected coordinates of or from any one of Tables 1-4 does not exclude the use of additional coordinates, particularly some or all of the coordinates of Table 5.

Furthermore, we have also found that another loop region, the B-B′ loop in the 3A4 co-complexes adopts a slightly different conformation than the conformation observed in the apo structure of 3A4. This is mostly side chain movement rather than main chain movement. The apo structure between residues Val95 and Phe102 could adopt the conformation seen for these residues in the co-complex structure. A further aspect or the invention is therefore where the B-B′ loop of the 3A4 apo structure of Table 1 or Table 4 has the conformation observed for residues Val95 to Phe102 of the 3A4 complexes of either of Tables 2 or 3. Thus in one aspect, the invention provides a crystal or crystal structure of P450 comprising amino acids having the atomic coordinates of Table 1 or Table 4, wherein the crystal alternatively comprises amino acids having the atomic coordinates of residues Val95 and Phe102 from Table 2 or Table 3.

Protein structure similarity is routinely expressed and measured by the root mean square deviation (r.m.s.d.), which measures the difference in positioning in space between two sets of atoms. The r.m.s.d. measures distance between equivalent atoms after their optimal superposition. The r.m.s.d. can be calculated over all atoms, over residue backbone atoms (i.e. the nitrogen-carbon-carbon backbone atoms of the protein amino acid residues), main chain atoms only (i.e. the nitrogen-carbon-oxygen-carbon backbone atoms of the protein amino acid residues), side chain atoms only or more usually over C-alpha atoms only. For the purposes of this invention, the r.m.s.d. can be calculated over any of these, using any of the methods outlined below.

Methods of comparing protein structures are discussed in Methods of Enzymology, vol 115, pg 397-420. The necessary least-squares algebra to calculate r.m.s.d. has been given by Rossman and Argos (J. Biol. Chem., vol 250, pp7525 (1975)) although faster methods have been described by Kabsch (Acta Crystallogr., Section A, A92, 922 (1976)); Acta Cryst. A34, 827-828 (1978)), Hendrickson (Acta Crystallogr., Section A, A35, 158 (1979)); McLachan (J. Mol. Biol., vol 128, pp49 (1979)) and Kearsley (Acta Crystallogr., Section A, A45, 208 (1989)). Some algorithms use an iterative procedure in which the one molecule is moved relative to the other, such as that described by Ferro and Hermans (Ferro and Hermans, Acta Crystallographic, A33, 345-347 (1977)). Other methods e.g. Kabsch's algorithm locate the best fit directly.

Programs for determining r.m.s.d include MNYFIT (part of a collection of programs called COMPOSER, Sutcliffe, M. J., Haneef, I., Carney, D. and Blundell, T. L. (1987) Protein Engineering, 1, 377-384), MAPS (Lu, G. An Approach for Multiple Alignment of Protein Structures (1998, in manuscript and on http://bioinfo1.mbfys.lu.se/TOP/maps.html)).

It is usual to consider C-alpha atoms and the rmsd can then be calculated using programs such as LSQKAB (Collaborative Computational Project 4. The CCP4 Suite: Programs for Protein Crystallography, Acta Crystallographica, D50, (1994), 760-763), QUANTA (Jones et al., Acta Crystallography A47 (1991), 110-119 and commercially available from Accelerys, San Diego, Calif.), Insight (commercially available from Accelerys, San Diego, Calif.), Sybyl® (commercially available from Tripos, Inc., St Louis), O (Jones et al., Acta Crystallographica, A47, (1991), 110-119), and other coordinate fitting programs.

In, for example the programs LSQKAB and O, the user can define the residues in the two proteins that are to be paired for the purpose of the calculation. Alternatively, the pairing of residues can be determined by generating a sequence alignment of the two proteins, programs for sequence alignment are discussed in more detail in Section F. The atomic coordinates can then be superimposed according to this alignment and an r.m.s.d. value calculated. The program Sequoia (C. M. Bruns, I. Hubatsch, M. Ridderström, B. Mannervik, and J. A. Tainer (1999) Human Glutathione Transferase A4-4 Crystal Structures and Mutagenesis Reveal the Basis of High Catalytic Efficiency with Toxic Lipid Peroxidation Products, Journal of Molecular Biology 288(3): 427-439) performs the alignment of homologous protein sequences, and the superposition of homologous protein atomic coordinates. Alternatively, the program Astex-KFIT (see Annex 1 and the subroutine of Annex 2) can be used. Once aligned, the r.m.s.d. can be calculated using programs detailed above. For sequence identical, or highly identical, the structural alignment of proteins can be done manually or automatically as outlined above. Another approach would be to generate a superposition of protein atomic coordinates without considering the sequence.

It is more normal when comparing significantly different sets of coordinates to calculate the r.m.s.d. value over C-alpha atoms only. It is particularly useful when analysing side chain movement to calculate the r.m.s.d. over all atoms and this can be done using LSQKAB and other programs.

Thus, for example, varying the atomic positions of the atoms of the structure by up to about 0.5 Å, preferably up to about 0.3 Å in any direction will result in a structure which is substantially the same as the structure of Table 1 in terms of both its structural characteristics and utility e.g. for molecular structure-based analysis. The same applies to Table 2, 3 and 4.

Those of skill in the art will appreciate that in many applications of the invention, it is not necessary to utilise all the coordinates of Tables 1-4, but merely a portion of them. For example, as described below, in methods of modelling candidate compounds with P450, selected coordinates of 3A4 may be used.

By “selected coordinates” it is meant for example at least 5, preferably at least 10, more preferably at least 50 and even more preferably at least 100, for example at least 500 or at least 1000 atoms of the 3A4 structure. Likewise, the other applications of the invention described herein, including homology modelling and structure solution, and data storage and computer assisted manipulation of the coordinates, may also utilise all or a portion of the coordinates (i.e. selected coordinates) of Tables 1-4. The selected coordinates may include or may consist of atoms found in the 3A4 P450 binding pocket, as described herein below, and particularly those of Table 6 and more particularly those of Table 7.

C. Description of Structure.

In the structure of 3A4 set out in Tables 1-4 herein, the first resolvable residue is Tyr25 and the last residue Gly498 (the protein as purified comprises residues residues 1, 2, and 25-503 of the wild type sequence (using wild type numbering from M18907) and a four histidine tag as shown in SEQ ID 2). The overall fold of the protein is typical of all P450 structures solved to date and the secondary structure elements are named according to the convention adopted for P450s Ravichandran, K. G., Boddupalli, S. S., Hasermann, C. A., Peterson, J. A., and Deisenhofer, J. (1993) Science 261, 731-736.

3A4 Apo Crystal

The overall structure of CYP3A4 conforms to the two-domain fold characteristic of the P450 family. The smaller N-terminal domain is predominantly beta strand while the larger, helical, C-terminal domain contains the haem and the active site. The haem iron is liganded by a conserved cysteine (Cys442) and the propionates of the haem interact with the side chains of Arg105, Trp126, Arg130, Arg375 and Arg440. The haem is accessible to solvent via a channel formed by beta sheet 1, the B-B′ loop and the B′ helix. The oxidation state of the haem iron in the structure is unknown, as while the protein used for crystallisation was oxidised and low spin, X-rays are capable of reducing haem iron. A low spin haem iron would be expected to have a ligand or water molecule in the sixth coordination position, but the structure reveals no ordered water bound in such a location. Another functionally important water molecule observed in other P450s is located between the conserved residues Ala305 and Thr309, which lie on the I helix and distal to the haem. In some CYP450 structures the threonine residue interacts with this water molecule, which disrupts the hydrogen-bonding pattern resulting in a kink in helix I (ref). Although a similar, but less pronounced, kink is observed in CYP3A4 there is no discrete density visible for this water molecule. In both cases, the resolution may explain why we are not able to identify these water molecules.

There are a number of distinguishing differences between previously solved P450 structures and the structure of 3A4. There is a short helix towards the N terminus (here denoted helix A″), not observed previously the mammalian P450 structures, before helix A′. This region, along with the G′ helix and the loop between the G′ and G helices, which are also hydrophobic in nature, may mediate interaction with the microsomal membrane. The B-C loop has less helical nature in 3A4 than in the previously solved human P450 2C9 structure (as contained in WO 03/035693 A2). This region along with the F-G loop region, has been implicated in forming an access channel (Podust, L. M., Stojan, J., Poulos, T. L., and Waterman, M. R. (2001) J Inorg Biochem 87, 227-235).

Another unexpected feature of the CYP3A4 structure is the region following a strikingly short helix F, which leads into a stretch of polypeptide chain that does not conform to any secondary structural motif. This region is located above and perpendicular to helix I, and contains a number of residues that have been shown by site directed mutagenesis to have a direct or indirect role in CYP3A4 function. For example, Leu210, implicated in effector binding as well as the stereo and regio-selectivity profile of CYP3A4, lies in this region with its side-chain pointing away from the substrate-binding-site. Leu211 and Asp214, which have also been implicated in cooperativity, also lie in this extended loop region and point away from the substrate-binding-site. The FG loop comprised 34 residues (210-243) and includes helix F′ and helix G′, compared to the 23 residues in the FG loop of 2C9. When compared to other P450s, the long FG loop of 3A4 is more due to the shortness of helix F than to the length of the FG loop itself. The B-C and F-G loops are in close proximity, forming two sides of the active site. It is widely accepted that 3A4 may bind several compound simultaneously, and can bind large compounds such erythromycin as well as compounds in excess of 1000 Da (e.g. cyclosporine). Movement of these regions may be required to allow the compound entry and egress, and they may become more structured if in alternative conformations. The loops between helices G and H, and helices H and I are not clearly resolved in the electron density maps (residues 261-270, 277-290) and have been excluded from the structure.

The dominating feature of the active site of substrate-free 3A4 is the cluster of phenylalanine residues (Phe57, Phe108, Phe213, Phe215, Phe219, Phe220, Phe241, Phe304) above the haem. Phe213 and Phe 215, which have been shown by mutagenesis to have no role in cooperativity, point towards the substrate binding region together with the remaining phenylalanines of the cluster. Some of these phenylalanines have been implicated by site directed mutagenesis to play a role in cooperativity and stereoselectivity. Phe304, which lies on the I helix, has a dual role in cooperativity, regio- and stereo-selectivity while the substitution of Phe108 with a smaller or larger amino acid affected the metabolism of some substrates.

The ‘Phe-cluster’ lies above the active site, with the aromatic side-chains stacking against each other, forming a prominent hydrophobic core. This region of the structure appears highly ordered and does not exhibit mobility as the average B factor of the residues in the cluster is 41 Å² compared with the average over the entire structure of 66 Å². Furthermore, as a result of this aromatic clustering, the active site of CYP3A4 has a volume almost half of that expected from homology modelling. In fact, the overall volume of the CYP3A4 active site is similar to that of CYP2C9, but has a different shape. The variation in the active site topologies is a consequence of the ‘Phe-cluster’, positioning the top of the active site closer to the haem, and a β-sheet being displaced away from the haem compared to CYP2C9. This results in the haem being more open to the active site, and could allow two substrate molecules to have access to the reactive oxygen, consistent with data that indicates CYP3A4 is able to bind and metabolise multiple substrate molecules simultaneously. Conformational movement involving the ‘Phe-cluster’ could reposition phenylalanine residues, perhaps extending helix F, and result in a larger active site, similar to the changes observed in an analogous region for CYP119 upon ligand binding.

Another cluster of four phenylalanine residues is found just below and to the side of the haem itself, in a position less clearly important for compound binding.

The kinetics exhibited by 3A4 can be complicated, with many literature examples citing one or more compound being accommodated simultaneously within the active site of 3A4 (Domanski et al, Biochemistry 2001, 40, 10150-10160). Site directed mutagenesis suggests that different substrates may bind at different regions of the active site. There is also evidence for homotropic cooperativity (interactions between a substrate and one or more effector molecules of the same chemical structure) and hetertropic cooperativity (where the substrate and effector molecules have different chemical structures).

Co-Crystals

To investigate whether conformational movement is a necessary prerequisite for ligand binding by CYP3A4 we first obtained the crystal structure complexed with a known inhibitor metyrapone. The metyrapone co-complex structure was determined using both soaking and, more importantly cocrystallisation techniques, to allow conformational movement by the protein if required.

Contrary to expectations, the binding of metyrapone to CYP3A4 reveals essentially no protein movement upon compound binding. UV/visable spectroscopic data (not shown) indicates that in solution the inhibitor is liganded to the haem, which is consistent with the binding mode observed in the crystal structure. Metyrapone is bound directly to the haem iron via a pyridine nitrogen and exhibits good shape complementarity as shown by the molecular surfaces. An alternative binding mode for metyrapone may involve the nitrogen atom of the other pyridine group, as observed previously in the co-complex with bacterial P450cam. However, the electron density for this CYP3A4 co-complex strongly supports the current binding mode as being predominant. The volume occupied by the metyrapone molecule is 50 Å³, leaving sufficient space for additional molecules to bind within the active site.

Although the metyrapone complex shows little conformational movement, we would anticipate that significant protein movement, possibly involving the F and G helical regions and the Phe-cluster, may be required to accommodate perhaps larger compounds. To investigate this possibility and also explore how CYP3A4 would recognise a substrate molecule, we determined the complex with progesterone by cocrystallisation. Surprisingly, we found the progesterone molecule induced very little conformational movement and bound at a peripheral site very close to the Phe-cluster, and some distance (>17 Å) away from the haem iron. The progesterone molecule forms a hydrogen bond between its acetyl oxygen and the amide nitrogen of Asp214 and packs against the side chains of Phe219 and Phe220, members of the ‘Phe-cluster’. Although this binding-site may be an artefact of crystallisation, we believe it more likely to have functional relevance, as several residues known to alter homo-cooperativity of progesterone are located in this region. For example, the side chains of both Leu211 and Asp214, residues implicated in effector binding, are in the vicinity of this progesterone binding pocket. Based on these findings we propose that the progesterone binding-site may be involved in the recognition of effector as well as substrate molecules and has a role in modulating cooperativity.

The location of the progesterone binding-site is also consistent with a role in initial substrate recognition. Appropriate conformational movement of residues around the Phe-cluster would result in a substrate access channel that stretched from this peripheral binding-site to the haem group, providing a route for a compound to move from this initial recognition site to the active site. Furthermore, this putative substrate-access channel is close to the F-G and B-C loops, regions previously implicated in this role. These conformational movements in the Phe-cluster could be triggered by interaction with a physiological electron-transfer partner such as cytochrome b₅, cytochrome P450 reductase or a change in membrane properties. Without such an interaction, the substrate molecule would remain bound at the initial recognition site as observed in the progesterone co-complex structure. Future studies to investigate these potential mechanisms involved in drug metabolism can now be guided by the crystal structure of this pharmacologically important protein.

Identification and Use of P450 Binding Pocket Residues.

The crystal structure for 3A4 has for the first time allowed the precise identification of all the residues that line the binding site of the enzyme (Table 6). Some residues proposed to be in the catalytic site by a variety of sources can now be shown not to be binding pocket residues but residues that hold the catalytic residues in place.

TABLE 6
below details all the residues that line the binding site of 3A4.

| | | | | | |
|---|---|---|---|---|---|
| Phe 57 | Asp 76 | Val 81 | Asn 104 | Arg 105 | Arg 106 |
| Pro 107 | Phe 108 | Gly 109 | Pro 110 | Val 111 | Met 114 |
| Ser 116 | Ala 117 | Ile 118 | Ser 119 | Ile 120 | Glu 122 |
| Thr 207 | Leu 210 | Leu 211 | Phe 2l5 | Phe 220 | Leu 221 |
| Ile 223 | Thr 224 | Ile 230 | Glu 234 | Val 235 | Leu 236 |
| Ile 238 | Cys 239 | Phe 241 | Pro 242 | Ala 297 | Ile 301 |
| Phe 302 | Ile 303 | Phe 304 | Ala 305 | Gly 306 | Glu 308 |
| Thr 309 | Ser 312 | Val 313 | Pro 368 | Ile 369 | Ala 370 |
| Met 371 | Arg 372 | Leu 373 | Glu 374 | Arg 375 | Ser 398 |
| Gly 481 | Leu 482 | Leu 483 | Glu 484 | | |

Some of these residues have previously been inferred to be in the binding site of 3A4 from modelling (e.g. homology modelling, SRS proposals, 3D/4D-QSAR, sequence alignments, or mutagenesis studies) which with the aid of the crystal structure can now be known to line the 3A4 binding pocket. Some residues found in the binding pocket have never before been identified as binding site residues. These are listed in Table 7. The identification of these will greatly facilitate the modelling of compound binding.

TABLE 7
Residues newly identified as lining the 3A4 binding pocket

| | | | | | |
|---|---|---|---|---|---|
| Phe 57 | Asp 76 | Val 81 | Arg 106 | Gly 109 | Pro 110 |
| Val 111 | Ser 116 | Ala 117 | Ile 118 | Glu 122 | Thr 207 |
| Phe 220 | Leu 221 | Ile 223 | Thr 224 | Ile 230 | Glu 234 |
| Val 235 | Leu 236 | Cys 239 | Phe 241 | Pro 242 | Ala 297 |
| Phe 302 | Ile 303 | Gly 306 | Ser 312 | Val 313 | Pro 368 |
| Arg 372 | Ser 398 | Gly 481 | Leu 482 | Leu 483 | Glu 484 |

Accordingly, in a preferred aspect of the invention, where the invention contemplates the use of selected coordinates in a method of the invention, such selected coordinates will comprise at least one coordinate, preferably at least one side-chain coordinate of an amino acid residue selected from either Table 6 or Table 7.

Preferably, the selected coordinates include the coordinates relating to at least one amino acid from Table 6 or Table 7 from any one of Tables 1-4.

Also preferred, whether all or just some atoms of a particular amino acid are selected, is that at least 2, more preferably at least 5, and most preferably at least 10 of the selected coordinates are of side chain residues from the corresponding number of different amino acid residues. These may be selected exclusively from either of Table 6 or Table 7, or a combination thereof. Preferably at least one side chain residue coordinate of Table 7 is included.

D. Chimeras.

The use of chimeric proteins to achieve desired properties is now common in the scientific literature. For example, Sieber et al (Nature Biotechnology (2001) 19, 456-460) produced hybrids between human cytochrome P450 isoform 1A2 and the bacterial P450 BM3, in order to make proteins with the specificity of 1A2, but which had desirable expression and solubility properties of BM3. Active site chimeras are also described: for example, Swairjo et al (Biochemistry (1998) 37, 10928-10936) made loop chimeras of HIV-1 and HIV-2 protease to try to understand determinants of inhibitor-binding specificity.

Of particular relevance are cases where the active site is modified so as to provide a surrogate system to obtain structural information. Thus Ikuta et al (J Biol Chem (2001) 276, 27548-27554) modified the active site of cdk2, for which they could obtain structural data, to resemble that of cdk4, for which no X-ray structure is currently available. In this way they were able to obtain protein/ligand structures from the chimaeric protein which were useful in cdk4 inhibitor design. In a similar way, based on comparison of primary sequences of highly related isoforms (such as 3A1, 3A5, 3A7, 3A12 or 3A43) the active site of the 3A4 protein could be modifie resemble those isoforms. Protein structures or protein/ligand structures of the chimaeric proteins could be used in structure-based alteration of the metabolism of compounds which are substrates of that related P450 isoform.

Even if the percentage of the amino acid sequence identity between mammalian P450 ranks from 20 to 80%, the overall folding of mammalian P450s is expected to be very similar, with the same spatial distribution of the structural elements. Furthermore, this class of enzymes exhibits distinct substrate specificities that rely on only a limited number of residues located in non-contiguous parts of the polypeptide chain. The substrate-binding pocket of P450 is generally constituted by residues that fall in the SRS regions (substrate recognition sites) defined by Gotoh (Gotoh, O, J. Biol. Chem, 267; 83-90 (1992)) and in loops of the molecule.

(i) Converting other P450 Proteins to 3A4-like Chimeras

Aspects of the present invention therefore relate to modification of P450 proteins such that the active sites mimic those of related isoforms. For example, from a knowledge of the structure and residues of the active site of the human 3A4 structure contained herein, a person skilled in the art could modify a P450 protein such that the active site mimicked that of human 3A4. This protein could then be used to obtain information on compound binding through the determination of protein/ligand complex structures using the chimaeric P450 protein.

For example, in one aspect the present invention provides a chimaeric protein having a binding cavity which provides a substrate specificity substantially identical to that of P450 3A4 protein, wherein the chimaeric protein binding cavity is lined by a plurality of atoms which correspond to selected P450 3A4 atoms lining the P450 3A4 binding cavity, and the relative positions of the plurality of atoms corresponding to the relative positions, as defined by any one of Tables 1-4, of the selected P450 3A4 atoms.

It is possible to postulate that only few changes would be required to inter-convert the substrate specificities of P450 isoforms that exhibit more than 70% of amino acid identity. 3A4 is 89% identical to 3A7, and 3A43 shares 76, 76, and 71% sequence identity on the amino acid level with CYP3A4, 3A5, and 3A7, respectively (Westlind et al, Biochemical and Biophysical Research Communications (2001), 281(5), 1349-1355; Gellner et al, Pharmacogenetics (2001), 11(2), 111-121). For example, although 3A4 and 3A5 are 84% identical they exhibit clear substrate specificity differences (Aoyama T; Yamano S; Waxman D J; Lapenson D P; Meyer U A; Fischer V; Tyndale R; Inaba T; Kalow W; Gelboin H V; Journal Of Biological Chemistry (1989 Jun. 25), 264(18), 10388-95). CYP3A4 is inhibited by mifepristone and yet CYP3A5 is not. Using a panel of 3A4/3A5 chimaeric proteins, Khan et al (Khan, Kishore K.; He, You Qun; Correia, Maria Almira; Halpert, James R; Drug Metabolism and Disposition (2002), 30(9), 985-990) have identified the sequence differences that explain the lack of inhibition of CYP3A5. These studies have demonstrated the feasibility of the transfer of substrate specificities between 3A4 and 3A5 by mutating residues within the SRS regions. CYP3A4 and CYP3A5 also show different regioselectivity towards aflatoxin B1 (AFB1) biotransformation, and a site-directed mutagenesis program to understand the structural features responsible for these differences, concluded that residues within the SRS region 2 alone were responsible for these differences (Huifen Wang, Ryan Dick, Hequn Yin, Estefania Licad-Coles, Deanna L. Kroetz, Grazyna Szklarz, Greg Harlow, James R. Halpert, and Maria Almira Correia, Biochemistry, 37 (36), 12536-12545, 1998).

The substrate specificity of an enzyme generally relies on only a limited number of residues located in non-contiguous parts of the polypeptide chain. The substrate specificities of these isoforms could be analysed by substituting these residues by site-directed mutagenesis. The minimal changes that would be required to convert another P450 protein into a 3A4-like chimera could be at least two amino acids selected from binding pocket, particularly the amino acid binding pocket residues of Table 6 or Table 7, more preferably Table 7. These mutations can be introduced by site-directed mutagenesis e.g. using a Stratagene QuikChange™ Site-Directed Mutagenesis Kit or cassette mutagenesis methods (Ausubel, F. M., Brent, R., Kingston, R. E. et al. editors. Current Protocols in Molecular Biology. John Wiley & Sons, Inc., New York, Sambrook, J., Fritsch, E. F., and Maniatis, T. (1989). Molecular Cloning: a Laboratory Manual. 2nd ed. Cold Spring Harbor Laboratory Press, Cold Spring Harbor, N.Y.). Thus the invention provides a chimaeric protein having one or more binding pockets defined by the residues of any one of Tables 1-4 and preferably including some or all of the binding pocket residues of Table 6 or Table 7.

(ii) Converting 3A4 to other 3A Isoforms

This strategy could clearly be applied for proteins that exhibit high sequence homology with or without overlapping substrate specificities and from different species. The use of the crystal structure solved for 3A4 would allow the characterization of the binding mode of a variety of molecules in the substrate pocket of these proteins. This in turn would allow the identification of residues to be modified in the human isoforms to convert them into metabolising enzymes with different substrate or regioselective preferences.

In one embodiment, a chimaeric 3A4 enzyme is produced which is isoformal with another enzyme of the 3A subfamily. For example, 3A4 could be turned into a 3A1-like, 3A5-like, 3A7 like, 3A12-like or 3A43-like isoform with a few amino acid changes. Based on the information available from the literature on the structure/activity studies performed on the human 3A4, 3A5, 3A7 and 3A43 isoforms, and the analysis of the structure of the human 3A4, we postulate that the 3A4 protein could be converted to a 3A5-like, 3A7-like or 3A43-like isoform with the substrate specificities attributed to 3A5, 3A7 or 3A43, 3A5 in particular based on the references above. The mutations can be introduced by site-directed mutagenesis or cassette mutagenesis methods, as described herein.

The crystallization of such chimeras and the determination of the three-dimensional structures relies on the ability of our 3A4 protein to yield crystals that diffract at high resolution. The aim is to modify the inside part of 3A4 to produce a new substrate binding site of 3A5, 3A7 or 3A43 without modifying the outside shell of the proteins that allow the protein to crystallize.

E. Homology Modelling.

The invention also provides a means for homology modelling of other proteins (referred to below as target P450 proteins). By “homology modelling”, it is meant the prediction of related P450 structures based either on X-ray crystallographic data or computer-assisted de novo prediction of structure, based upon manipulation of the coordinate data derivable from any one of Tables 1-4 or selected portions thereof.

“Homology modelling” extends to target P450 proteins which are analogues or homologues of the 3A4 protein whose structure has been determined in the accompanying examples. It also extends to P450 protein mutants of 3A4 protein itself.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being respectively “invariant” and “conserved” by those skilled in the art.

In general, the method involves comparing the amino acid sequences of the 3A4 protein of SEQ ID 2 with a target P450 protein by aligning the amino acid sequences. Amino acids in the sequences are then compared and groups of amino acids that are homologous (conveniently referred to as “corresponding regions”) are grouped together. This method detects conserved regions of the polypeptides and accounts for amino acid insertions or deletions.

Homology between amino acid sequences can be determined using commercially available algorithms. The programs BLAST, gapped BLAST, BLASTN, PSI-BLAST and BLAST 2 (provided by the National Center for Biotechnology Information) are widely used in the art for this purpose, and can align homologous regions of two amino acid sequences. These may be used with default parameters to determine the degree of homology between the amino acid sequence of the SEQ ID 2 protein and other target P450 proteins which are to be modelled.

Analogues are defined as proteins with similar three-dimensional structures and/or functions with little evidence of a common ancestor at a sequence level.

Homologues are defined as proteins with evidence of a common ancestor, i.e. likely to be the result of evolutionary divergence and are divided into remote, medium and close sub-divisions based on the degree (usually expressed as a percentage) of sequence identity.

A homologue is defined here as a protein with at least 15% sequence identity or which has at least one functional domain, which is characteristic of 3A4. This includes polymorphic forms of 3A4.

There are two types of homologue: orthologues and paralogues. Orthologues are defined as homologous genes in different organisms, i.e. the genes share a common ancestor coincident with the speciation event that generated them. Paralogues are defined as homologous genes in the same organism derived from a gene/chromosome/genome duplication, i.e. the common ancestor of the genes occurred since the last speciation event.

The homlogues could also be polymorphic forms of 3A4 such as alleles or mutants as described in section (A).

Once the amino acid sequences of the polypeptides with known and unknown structures are aligned, the structures of the conserved amino acids in a computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions may be assigned manually by using standard peptide geometries or by molecular simulation techniques, such as molecular dynamics. The final step in the process is accomplished by refining the entire structure using molecular dynamics and/or energy minimization.

Homology modelling as such is a technique that is well known to those skilled in the art (see e.g. Greer, Science, Vol. 228, (1985), 1055, and Blundell et al., Eur. J. Biochem, Vol. 172, (1988), 513). The techniques described in these references, as well as other homology modelling techniques, generally available in the art, may be used in performing the present invention.

Thus the invention provides a method of homology modelling comprising the steps of:
(a) aligning a representation of an amino acid sequence of a target P450 protein of unknown three-dimensional structure with the amino acid sequence of the P450 of SEQ ID 2 to match homologous regions of the amino acid sequences;
(b) modelling the structure of the matched homologous regions of said target P450 of unknown structure on the corresponding regions of the P450 structure as obtained as described above and/or that of any one of Tables 1-4 or selected coordinates thereof; and
(c) determining a conformation (e.g. so that favourable interactions are formed within the target P450 of unknown structure and/or so that a low energy conformation is formed) for said target P450 of unknown structure which substantially preserves the structure of said matched homologous regions.

Preferably one or all of steps (a) to (c) are performed by computer modelling.

The co-ordinate data of Tables 1-4 or selected coordinates thereof, will be particularly advantageous for homology modelling of other human P450 proteins, in particular human P450s such as 2C9, 2C19, 2D6, 3A5, 3A7, 1A1, 1A2, 2E1 preferably 3A5, 3A7 and 3A43. These proteins may be the target P450 protein in the method of the invention described above.

The aspects of the invention described herein which utilise the P450 structure in silico may be equally applied to homologue models of P450 obtained by the above aspect of the invention, and this application forms a further aspect of the present invention. Thus having determined a conformation of a P450 by the method described above, such a conformation may be used in a computer-based method of rational drug design as described herein.

F. Structure Solution

The atomic coordinate data of 3A4 can also be used to solve the crystal structure of other target P450 proteins including other crystal forms of 3A4, mutants, co-complexes of 3A4, where X-ray diffraction data or NMR spectroscopic data of these target P450 proteins has been generated and requires interpretation in order to provide a structure.

In the case of 3A4, this protein may crystallize in more than one crystal form. The data of Tables 1-4, or portions thereof, as provided by this invention, are particularly useful to solve the structure of those other crystal forms of 3A4. It may also be used to solve the structure of 3A4 mutants, 3A4 co-complexes, or of the crystalline form of any other protein with significant amino acid sequence homology to any functional domain of 3A4.

In the case of other target P450 proteins, particularly the human P450 proteins referred to in Section E above, the present invention allows the structures of such targets to be obtained more readily where raw X-ray diffraction data is generated.

Thus, where X-ray crystallographic or NMR spectroscopic data is provided for a target P450 of unknown three-dimensional structure, the atomic coordinate data derived from any one of Tables 1-4, may be used to interpret that data to provide a likely structure for the other P450 by techniques which are well known in the art, e.g. phasing in the case of X-ray crystallography and assisting peak assignments in NMR spectra.

One method that may be employed for these purposes is molecular replacement. In this method, the unknown crystal structure, whether it is another crystal form of 3A4, a 3A4 mutant, a 3A4 chimera or an 3A4 co-complex, or the crystal of a target P450 protein with amino acid sequence homology to any functional domain of 3A4, may be determined using the 3A4 structure coordinates of all or part of any one of Tables 1-4 of this invention. This method will provide an accurate structural form for the unknown crystal more quickly and efficiently than attempting to determine such information ab initio.

Examples of computer programs known in the art for performing molecular replacement are CNX (Brunger A. T.; Adams P. D.; Rice L. M., Current Opinion in Structural Biology, Volume 8, Issue 5, October 1998, Pages 606-611 (also commercially available from Accelrys San Diego, Calif.), MOLREP (A. Vagin, A. Teplyakov, MOLREP: an automated program for molecular replacement, J. Appl. Cryst. (1997) 30, 1022-1025, part of the CCP4 suite) or AMoRe (Navaza, J. (1994). AMoRe: an automated package for molecular replacement. Acta Cryst. A50, 157-163).

Thus, in a further aspect of the invention provides a method for determining the structure of a protein, which method comprises;
providing the coordinates (or selected coordinates thereof) of the 3A4 structure of any one of Tables 1-4,
positioning the coordinates in the crystal unit cell of said protein so as to provide a structure for said protein.

Preferably the coordinates of Tables 1-4 or selected coordinates thereof, include coordinates of atoms of the amino acid residues set out in Table 6 and more preferably in Table 7.

The invention may also be used to assign peaks of NMR spectra of such proteins, by manipulation of the data of any one of Tables 1-4.

In a preferred aspect of this invention the co-ordinates are used to solve the structure of target 3A4 particularly homologues of 3A4 for example P450s such as 3A5, 3A7 and 3A43.

G. Computer Systems.

In another aspect, the present invention provides systems, particularly a computer system, the systems containing one of (a) 3A4 co-ordinate data of any one of Tables 1-4, said data defining the three-dimensional structure of P450 or at least selected coordinates thereof; (b) atomic coordinate data of a target P450 protein generated by homology modelling of the target based on the coordinate data of any one of Tables 1-4, (c) atomic coordinate data of a target P450 protein generated by interpreting X-ray crystallographic data or NMR data by reference to the co-ordinate data of any one of Tables 1-4; or (d) structure factor data derivable from the atomic coordinate data of (b) or (c).

For example the computer system may comprise: (i) a computer-readable data storage medium comprising data storage material encoded with the computer-readable data; (ii) a working memory for storing instructions for processing said computer-readable data; and (iii) a central-processing unit coupled to said working memory and to said computer-readable data storage medium for processing said computer-readable data and thereby generating structures and/or performing rational drug design. The computer system may further comprise a display coupled to said central-processing unit for displaying said structures.

The invention also provides such systems containing atomic coordinate data of target P450 proteins wherein such data has been generated according to the methods of the invention described herein based on the starting data provided the data of any one of Tables 1-4 or selected coordinates thereof.

Such data is useful for a number of purposes, including the generation of structures to analyse the mechanisms of action of P450 proteins and/or to perform rational drug design of compounds, which interact with P450, such as compounds, which are metabolised by P450s.

In a further aspect, the present invention provides computer readable media with at least one of (a) 3A4 co-ordinate data of any one of Tables 1-4, said data defining the three-dimensional structure of P450 or at least selected coordinates thereof; (b) atomic coordinate data of a target P450 protein generated by homology modelling of the target based on the coordinate data of any one of Tables 1-4, (c) atomic coordinate data of a target P450 protein generated by interpreting X-ray crystallographic data or NMR data by reference to the co-ordinate data of any one of Tables 1-4; or (d) structure factor data derivable from the atomic coordinate data of (b) or (c).

In another aspect, the invention provides a computer-readable storage medium, comprising a data storage material encoded with computer readable data, wherein the data are defined by all or a portion (e.g. selected coordinates as defined herein) of the structure coordinates of P450 of any one of Tables 1-4, or a homologue of said P450, wherein said homologue comprises backbone atoms that have a root mean square deviation from the Cα or backbone atoms (nitrogen-carbon_{α}-carbon) of any one of Tables 1-4 of less than 2 Å, preferably less than 1.55 or 1.5 Å, more preferably less than 1.0 Å, and most preferably less than 0.5 Å.

As used herein, “computer readable media” refers to any medium or media, which can be read and accessed directly by a computer. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By providing such computer readable media, the atomic coordinate data of the invention can be routinely accessed to model P450s or selected coordinates thereof. For example, RASMOL (Sayle et al., TIBS, Vol. 20, (1995), 374) is a publicly available computer software package, which allows access and analysis of atomic coordinate data for structure determination and/or rational drug design.

As used herein, “a computer system” refers to the hardware means, software means and data storage means used to analyse the atomic coordinate data of the invention. The minimum hardware means of the computer-based systems of the present invention comprises a central processing unit (CPU), input means, output means and data storage means. Desirably a monitor is provided to visualize structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

The invention also provides a computer-readable data storage medium comprising a data storage material encoded with a first set of computer-readable data comprising the 3A4 coordinates of any one of Tables 1-4 or selected coordinates thereof; which, when combined with a second set of machine readable data comprising an X-ray diffraction pattern of a molecule or molecular complex of unknown structure, using a machine programmed with the instructions for using said first set of data and said second set of data, can determine at least a portion of the electron density corresponding to the second set of machine readable data.

A further aspect of the invention provides a method of providing data for generating structures and/or performing rational drug redesign with 3A4, 3A4 homologues or analogues, complexes of 3A4 with a compound, or complexes of 3A4 homologues or analogues with compounds, the method comprising:
(i) establishing communication with a remote device containing computer-readable data comprising at least one of: (a) co-ordinate data defining the three-dimensional structure of 3A4, at least one sub-domain of the three-dimensional structure of 3A4, or the coordinates of a plurality of atoms of 3A4, said coordinate data being the 3A4 coordinate data of any one of Tables 1-4; (b) atomic coordinate data of a target 3A4 homologue or analogue generated by homology modelling of the target based on the coordinate data of any one of Tables 1-4; (c) atomic coordinate data of a protein generated by interpreting X-ray crystallographic data or NMR data by reference to the data of any one of Tables 1-4; and (d) structure factor data derivable from the atomic coordinate data of (b) or (c); and
(ii) receiving said computer-readable data from said remote device.

The atomic coordinate data may include coordinates of amino acids set out in Tables 6 or 7.

Thus the remote device may comprise e.g. a computer system or computer readable media of one of the previous aspects of the invention. The device may be in a different country or jurisdiction from where the computer-readable data is received.

The communication may be via the intemet, intranet, e-mail etc, transmitted through wires or by wireless means such as by terrestrial radio or by satellite. Typically the communication will be electronic in nature, but some or all of the communication pathway may be optical, for example, over optical fibers.

H. Uses of the Structures of the Invention.

The crystal structures obtained according to the present invention as well as the structures of target P450 proteins obtained in accordance with the methods described herein), may be used in several ways for drug design. For example, many drugs or drug candidates fail to be of clinical use due to the detrimental interactions with P450 proteins, resulting in a rapid clearance of the drugs from the body. The present invention will allow those of skill in the art to attempt to rescue such compounds from development, by following the structure-based chemical strategies detailed below.

In the case where a drug molecule is being metabolised by a P450, information on the binding orientation by either co-crystallization, soaking or computationally docking the binding orientation of the drug in the binding pocket can be determined. This will guide specific modifications to the chemical structure designed to mediate or control the interaction of the drug with the protein. Such modifications can be designed with an aim to reduce the metabolism of the drug by P450 and so improve its therapeutic action.

The crystal structure could also be useful to understand drug-drug interactions. Many examples exist where adverse reactions to drugs are recorded if administered while the patient is already taking other medicines. The mechanism behind this detrimental and often dangerous drug-drug interaction scenario may be when one drug behaves as an inhibitor of a P450 resulting in toxic levels of the other drug building-up due to less or no metabolism occurring. The crystal structure of the present invention complexed to such an inhibitor (either in vitro or in silico) may also allow rational modifications either to modify the inhibitor such that it no longer inhibits or inhibits less, or to modify the second drug such that it could bind better to the P450 (so becoming metabolised) and so displace the inhibitor.

P450s display significant polymorphic variations dependent on the age, gender, or ethnic origin of the patient. This can manifest itself in adverse reactions from some segments of patient populations to some drugs. By using the crystal structures of the present invention to map the relevant mutation with respect to the binding mode of the drug, chemical modifications could also be made to the drug to avoid interactions with the variable region of the protein. This could ensure more consistent therapeutic value from the drug for such segments of the population and avoid dangerous side effects.

Some pharmaceutical compounds are converted by P450s into active metabolites. In the case of such compounds, a greater understanding of how such compounds are converted by a P450 will allow modification of the compound so that it can be converted at a different rate. For example, increasing the rate of conversion may allow a more rapid delivery of a desired therapeutic effect, whereas decreasing the rate of conversion may allow for higher doses to be administered or the development of sustained release pharmaceutical preparations, for example comprising a mixture of compounds which are metabolized at different rates to form the same active metabolite.

Thus, the determination of the three-dimensional structure of P450 provides a basis for the design of new compounds, which interact with P450 in novel ways. For example, knowing the three-dimensional structure of P450, computer modelling programs may be used to design different molecules expected to interact with possible or confirmed active sites, such as binding sites or other structural or functional features of P450.

(i) Obtaining and Analysing Crystal Complexes.

In one approach, the structure of a compound bound to a P450 may be determined by experiment. This will provide a starting point in the analysis of the compound bound to P450, thus providing those of skill in the art with a detailed insight as to how that particular compound interacts with P450 and the mechanism by which it is metabolised.

Many of the techniques and approaches to structure-based drug design described above rely at some stage on X-ray analysis to identify the binding position of a ligand in a ligand-protein complex. A common way of doing this is to perform X-ray crystallography on the complex, produce a difference Fourier electron density map, and associate a particular pattern of electron density with the ligand. However, in order to produce the map (as explained e.g. by Blundell et al., in Protein Crystallography, Academic Press, New York, London and San Francisco, (1976)), it is necessary to know beforehand the protein 3D structure (or at least the protein structure factors). Therefore, determination of the P450 structure also allows difference Fourier electron density maps of P450-compound complexes to be produced, determination of the binding position of the drug and hence may greatly assist the process of rational drug design.

Accordingly, the invention provides a method for determining the structure of a compound bound to P450, said method comprising:
providing a crystal of P450 according to the invention;
soaking the crystal with said compounds; and
determining the structure of said P450 compound complex by employing the coordinate data of any one of Tables 1-4 or selected coordinates thereof.

Alternatively, the P450 and compound may be co-crystallized. Thus the invention provides a method for determining the structure of a compound bound to P450, said method comprising; mixing the protein with the compound(s), crystallizing the protein-compound(s) complex; and determining the structure of said P450-compound(s) complex by reference to the coordinate data of any one of Tables 1-4 or selected coordinates thereof.

The analysis of such structures may employ (i) X-ray crystallographic diffraction data from the complex and (ii) a three-dimensional structure of P450, or at least selected coordinates thereof, to generate a difference Fourier electron density map of the complex, the three-dimensional structure being defined by atomic coordinate data of any one of Tables 1-4 or selected coordinates thereof. The difference Fourier electron density map may then be analysed.

Therefore, such complexes can be crystallized and analysed using X-ray diffraction methods, e.g. according to the approach described by Greer et al., J. of Medicinal Chemistry, Vol. 37, (1994), 1035-1054, and difference Fourier electron density maps can be calculated based on X-ray diffraction patterns of soaked or co-crystallized P450 and the solved structure of uncomplexed P450. These maps can then be analysed e.g. to determine whether and where a particular compound binds to P450 and/or changes the conformation of P450.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualization and model building programs such as “O” (Jones et al., Acta Crystallographica, A47, (1991), 110-119) can be used.

In addition, in accordance with this invention, 3A4 mutants may be crystallized in co-complex with known 3A4 substrates or inhibitors or novel compounds. The crystal structures of a series of such complexes may then be solved by molecular replacement and compared with that of the 3A4 structure of any one of Tables 1-4 or selected coordinates thereof. Potential sites for modification within the various binding sites of the enzyme may thus be identified. This information provides an additional tool for determining the most efficient binding interactions, for example, increased hydrophobic interactions, between 3A4 and a chemical entity or compound.

For example there are alleles of 3A4, which differ from the native 3A4 by only 1-2 amino acid substitutions, and yet individuals who express these allelic variants may exhibit very different drug metabolism profiles. Polymorphisms in the human CYP3A4 genes can influence the outcome of a treatment for a range of diseases including cancer. The metabolism of chemotherapeutic agents used in the treatment of cancer can be investigated using the structure provided here and the agents then altered using the methods described herein.

The methods described herein could be used to design prodrugs which are activated by 3A4. CYP3A4 plays a major role in the activation of procarcinogens such as polycyclic hydrocarbon dihydrodiols, aflatoxins and heterocyclic amines as well as of several drugs including tamoxifen which is used in breast cancer therapy. The level of expression of CYP3A4 in breast tumour and surrounding tumour free (control) breast tissue showed that CYP3A4 levels were found to be significantly higher in tumours compared to that of normal breast tissues. These results show that CYP3A4 protein is expressed in both tumour and normal breast tissue with an increased expression in tumours. (Nilgun et al, 2003, Cancer Letters, 202(1), 17-23). As 3A4 is expressed at higher levels in tumour cells than non-tumour cells, it may present a cancer prodrug opportunity. Cyclophosphamide, ifosfamide and other nitrogen mustard prodrugs chemotherapy compounds are activated by 4-hydroxylation catalysed by CYP3A4 and other P450s. Alkylaminoanthraquinone 1,4-bis-((2-(dimethyl-amino-N-oxide)ethyl)amoni)-5,8-dihydroxyanthracene-9,10-dione (AQ4N) is activated by CYP3A and other isoforms into a high-affinity DNA-binding compound capable of inhibiting topoisomerase II inhibitor, AQ4 (Raleigh et al, 1999, Int J Radiat Oncol Biol Phys, 42, 763-767).

CYPs are also implicated in myocardial ischemia/reperfusion injury and reduction of ischemia and reperfusion-induced myocardial damage has been observed by cytochrome P450 inhibitors, thus the methods described herein could be used to cytochrome P450 inhibitors for reduction of ischemia and reperfusion-induced myocardial damage.

By generating such allelic proteins and determining the co-complex with compounds a greater understanding of allelic interactions with compounds may be developed.

All of the complexes referred to above may be studied using well-known X-ray diffraction techniques and may be refined against 1.5 to 3.5 Å resolution X-ray data to an R value of about 0.30 or less using computer software, such as CNX (Brunger et al., Current Opinion in Structural Biology, Vol. 8, Issue 5, October 1998, 606-611, and commercially available from Accelrys, San Diego, Calif.), and as described by Blundell et al, (1976) and Methods in Enzymology, vol. 114 & 115, H. W. Wyckoff et al., eds., Academic Press (1985).

This information may thus be used to optimise known classes of 3A4 substrates or inhibitors, and more importantly, to design and synthesize novel classes of 3A4 inhibitors and design drug with modified P450 metabolism.

(ii) In Silico Analysis and Design

Although the invention will facilitate the determination of actual crystal structures comprising a P450 and a compound, which interacts with the P450, current computational techniques provide a powerful alternative to the need to generate such crystals and generate and analyse diffraction date. Accordingly, a particularly preferred aspect of the invention relates to in silico methods directed to the analysis and development of compounds which interact with P450 structures of the present invention.

Determination of the three-dimensional structure of 3A4 provides important information about the binding sites of 3A4, particularly when comparisons are made with similar enzymes. This information may then be used for rational design and modification of 3A4 substrates and inhibitors, e.g. by computational techniques which identify possible binding ligands for the binding sites, by enabling linked-fragment approaches to drug design, and by enabling the identification and location of bound ligands using X-ray crystallographic analysis. These techniques are discussed in more detail below.

Thus as a result of the determination of the P450 three-dimensional structure, more purely computational techniques for rational drug design may also be used to design structures whose interaction with P450 is better understood (for an overview of these techniques see e.g. Walters et al (Drug Discovery Today, Vol. 3, No. 4, (1998), 160-178; Abagyan, R.; Totrov, M. Curr. Opin. Chem. Biol. 2001, 5, 375-382). For example, automated ligand-receptor docking programs (discussed e.g. by Jones et al. in Current Opinion in Biotechnology, Vol. 6, (1995), 652-656 and Halperin, I.; Ma, B.; Wolfson, H.; Nussinov, R. Proteins 2002, 47, 409-443), which require accurate information on the atomic coordinates of target receptors may be used.

The aspects of the invention described herein which utilize the P450 structure in silico may be equally applied to both the 3A4 structure of any one of Tables 1-4 or selected coordinates thereof and the models of target P450 proteins obtained by other aspects of the invention. Thus having determined a conformation of a P450 by the method described above, such a conformation may be used in a computer-based method of rational drug design as described herein. In addition the availability of the structure of the P450 3A4 will allow the generation of highly predictive pharmacophore models for virtual library screening or compound design.

Accordingly, the invention provides a computer-based method for the analysis of the interaction of a molecular structure with a P450 structure of the invention, which comprises:
providing the structure of a P450 of the invention;
providing a molecular structure to be fitted to said P450 structure; and
fitting the molecular structure to the P450 structure.

The P450 structure of the invention may be that of any one of Tables 1-4, or selected coordinates thereof.

In an alternative aspect, the method of the invention may utilize the coordinates of atoms of interest of the P450 binding region, which are in the vicinity of a putative molecular structure, for example within 10-25 Å of the catalytic regions or within 5-10 Å of a compound bound, in order to model the pocket in which the structure binds. These coordinates may be used to define a space, which is then analysed “in silico”. Thus the invention provides a computer-based method for the analysis of molecular structures which comprises:
providing the coordinates of at least two atoms of a P450 structure of the invention (“selected coordinates”);
providing the structure of a molecular structure to be fitted to said coordinates; and
fitting the structure to the selected coordinates of the P450.

In practice, it will be desirable to model a sufficient number of atoms of the P450 as defined by the coordinates of any one of Tables 1-4 or selected coordinates thereof), which represent a binding pocket, e.g. the atoms of the residues identified in Tables 6 and 7, preferably Table 7. Binding pockets and other features of the interaction of P450 with co-factor are described in the accompanying example. Thus, in this embodiment of the invention, there will preferably be provided the coordinates of at least 5, preferably at least 10, more preferably at least 50 and even more preferably at least 100, e.g. at least 500 such as at least 1000, selected atoms of the P450 structure.

Although every different compound metabolised by P450 may interact with different parts of the binding pocket of the protein, the structure of this P450 allows the identification of a number of particular sites which are likely to be involved in many of the interactions of P450 with a drug candidate. The residues are set out in Tables 6 and 7. Thus in this aspect of the invention, the selected coordinates may comprise coordinates of some or all of these residues.

In order to provide a three-dimensional structure of compounds to be fitted to a P450 structure of the invention, the compound structure may be modelled in three dimensions using commercially available software for this purpose or, if its crystal structure is available, the coordinates of the structure may be used to provide a representation of the compound for fitting to a P450 structure of the invention.

The binding pockets of cytochrome P450 molecules are of a size which can accommodate more than one ligand. Indeed, some drug-drug interactions may occur as a result of interaction of the compounds within the binding pocket of the same P450. In any event, the findings of the present invention may be used to examine or predict the interaction of two or more separate molecular structures within the P450 3A4 binding pocket of the invention.

Thus the invention provides a computer-based method for the analysis of the interaction of two molecular structures within a P450 binding pocket structure, which comprises:
providing the P450 structure of any one of Tables 1-4 or selected coordinates thereof;
providing a first molecular structure;
fitting the first molecular structure to said P450 structure;
providing a second molecular structure; and
fitting the second molecular structure to a different part said P450 structure.

Optionally the method of analysis further comprises providing a third molecular structure and also fitting that structure to the P450 structure. Indeed, further molecular structures may be provided and fitted in the same way.

In one aspect, one or more of the molecular structures may be fitted to one or more of the phenylalanine residues of the 3A4 binding pocket mentioned above, and one or more of the other molecular structures may be fitted to coordinates of amino acids from another part of the P450 binding pocket, such as another part of the ligand-binding region, to the haem-binding region, or to atoms of the amino acid residues of Tables 6 or 7. In one embodiment, the one or more other molecular structures may be fitted, in addition to or instead of, to the haem structure in the P450 binding pocket.

Following the fitting of the molecular structures, a person of skill in the art may seek to use molecular modelling to determine to what extent the structures interact with each other (e.g. by hydrogen bonding, other non-covalent interactions, or by reaction to provide a covalent bond between parts of the structures) or the interaction of one structure with 3A4 is altered by the presence of another structure.

The person of skill in the art may use in silico modelling methods to alter one or more of the structures in order to design new structures which interact in different ways with 3A4, so as to speed up or slow down their metabolism, as the case may be.

Newly designed structures may be synthesised and their interaction with 3A4 may be determined or predicted as to how the newly designed structure is metabolised by said P450 structure. This process may be iterated so as to further alter the interaction between it and the 3A4.

By “fitting”, it is meant determining by automatic, or semi-automatic means, interactions between at least one atom of a molecular structure and at least one atom of a P450 structure of the invention, and calculating the extent to which such an interaction is stable. Interactions include attraction and repulsion, brought about by charge, steric considerations and the like. Various computer-based methods for fitting are described further herein.

More specifically, the interaction of a compound or compounds with P450 can be examined through the use of computer modelling using a docking program such as GOLD (Jones et al., J. Mol. Biol., 245, 43-53 (1995), Jones et al., J. Mol. Biol., 267, 727-748 (1997)), GRAMM (Vakser, I. A., Proteins, Suppl., 1:226-230 (1997)), DOCK (Kuntz et al, J. Mol. Biol. 1982, 161, 269-288, Makino et al, J.Comput.Chem. 1997, 18,1812-1825), AUTODOCK (Goodsell et al, Proteins 1990, 8, 195-202, Morris et al, J. Comput. Chem. 1998, 19, 1639-1662.), FlexX, (Rarey et al, J.Mol.Biol. 1996, 261, 470-489) or ICM (Abagyan et al, J. Comput. Chem. 1994, 15, 488-506). This procedure can include computer fitting of compounds to P450 to ascertain how well the shape and the chemical structure of the compound will bind to the P450.

Also computer-assisted, manual examination of the active site structure of P450 may be performed. The use of programs such as GRID (Goodford, J. Med. Chem., 28, (1985), 849-857)—a program that determines probable interaction sites between molecules with various functional groups and an enzyme surface-may also be used to analyse the active site to predict, for example, the types of modifications which will alter the rate of metabolism of a compound.

Computer programs can be employed to estimate the attraction, repulsion, and steric hindrance of the two binding partners (i.e. the P450 and a compound).

If more than one P450 active site is characterized and a plurality of respective smaller compounds are designed or selected, a compound may be formed by linking the respective small compounds into a larger compound, which maintains the relative positions and orientations of the respective compounds at the active sites. The larger compound may be formed as a real molecule or by computer modelling.

Detailed structural information can then be obtained about the binding of the compound to P450, and in the light of this information adjustments can be made to the structure or functionality of the compound, e.g. to alter its interaction with P450. The above steps may be repeated and re-repeated as necessary.

As indicated above, molecular structures, which may be fitted to the P450 structure of the invention, include compounds under development as potential pharmaceutical agents. The agents may be fitted in order to determine how the action of P450 modifies the agent and to provide a basis for modelling candidate agents, which are metabolised at a different rate by a P450.

Molecular structures, which may be used in the present invention, will usually be compounds under development for pharmaceutical use. Generally such compounds will be organic molecules, which are typically from about 100 to 2000 Da, more preferably from about 100 to 1000 Da in molecular weight. Such compounds include peptides and derivatives thereof, steroids, anti-inflammatory drugs, anti-cancer agents, anti-bacterial or antiviral agents, neurological agents and the like. In principle, any compound under development in the field of pharmacy can be used in the present invention in order to facilitate its development or to allow further rational drug design to improve its properties.

(iii) Analysis and Modification of Compounds and Metabolites

Where the primary metabolite of a potential or actual pharmaceutical compound is known, and this metabolite is generated by the action of P450, the structure of the agent and its metabolite may both be modelled and compared to each other in order to better determine residues of P450 which interact with the agent. In any event, the present invention provides a process for predicting potential pharmaceutical compounds with a desired activity which are metabolised by P450 at a rate different from a starting compound having the same desired activity, which method comprises:
fitting a starting compound to a P450 structure of the invention or selected coordinates thereof;
determining or predicting how said compound is metabolized by said P450 structure; and modifying the compound structure so as to alter the interaction between it and the P450.

It would be understood by those of skill in the art that modification of the structure will usually occur in silico, allowing predictions to be made as to how the modified structure interacts with the P450.

Greer et al. (J. of Medicinal Chemistry, Vol. 37, (1994), 1035-1054) describes an iterative approach to ligand design based on repeated sequences of computer modelling, protein-ligand complex formation and X-ray crystallographic or NMR spectroscopic analysis. Thus novel thymidylate synthase inhibitor series were designed de novo by Greer et al., and P450 ligands may also be designed or modified in the this way. More specifically, using e.g. GRID on the solved structure of P450, a ligand for P450 may be designed that complements the functionalities of the P450 binding sites. Alternatively a ligand for P450 may be modified such that it complements the functionalities of the P450 binding sites better or less well. The ligand can then be synthesised, formed into a complex with P450, and the complex then analysed by X-ray crystallography to identify the actual position of the bound ligand. The structure and/or functional groups of the ligand can then be adjusted, if necessary, in view of the results of the X-ray analysis, and the synthesis and analysis sequence repeated until an optimised ligand is obtained. Related approaches to structure-based drug design are also discussed in Bohacek et al., Medicinal Research Reviews, Vol. 16, (1996), 3-50. Design of a compound with alternative P450 properties using structure based drug design may also take into account the requirements for high affinity to a second, target protein. Gschwend et al., (Bioorganic & Medicinal Chemistry Letters, Vol 9, (1999), 307-312) and Bayley et al., (Proteins: Structure, Function and Genetics, Vol 29, (1997) 29-67) describe approaches where structure based drug design is used to reduce affinity to one protein whilst maintaining affinity for a target protein.

Modification will be those conventional in the art known to the skilled medicinal chemist, and will include, for example, substitutions or removal of groups containing residues which interact with the amino acid side chain groups of a P450 structure of the invention. For example, the replacements may include the addition or removal of groups in order to decrease or increase the charge of a group in a test compound, the replacement of a group to increase or decrease the size of the group in a test compound, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting compound and its activity.

Although it is usually desired to alter a compound to prevent its metabolism by P450, or at least to reduce the rate at which P450 metabolises the compound, the present invention also includes developing compounds which are metabolised more rapidly than a starting compound. Additionally the present invention includes developing compounds with high affinity for a P450, where such a compound blocks metabolism of another drug.

Where a potential modified compound has been developed by fitting a starting compound to the P450 structure of the invention and predicting from this a modified compound with an altered rate of metabolism, the invention further includes the step of synthesizing the modified compound and testing it in a in vivo or in vitro biological system in order to determine its activity and/or the rate at which it is metabolised.

The above-described processes of the invention may be iterated in that the modified compound may itself be the basis for further compound design. The above-described processes may also be used to modify a compound which interacts with a second compound within the 3A4 binding pocket.

(iv) Analysis of Compounds in Binding Pocket Regions

Our finding of a cluster of phenylalanine residues in the vicinity of the haem of 3A4 allows the analysis and design methods described in the preceding subsections to be focused on compounds which interact with one or more of these residues.

For example, compounds which dock in the 3A4 substrate binding pocket in a manner which includes pi:pi stacking interactions with a phenylalanine side chain, may be modified in order to alter their metabolism. For example, such interactions may be influential in determining the rate at which the compounds undergo metabolism via movement towards, and reaction with, the haem moiety, located in the haem binding region of the 3A4 binding pocket. By altering (i.e. increasing or decreasing) their affinity of the compound to these phenylalanine residues, or other features of the ligand binding region compared to the haem binding region it may alter (i.e. increase or decrease) their ability to move towards, or be retained by, the haem-binding region.

For example by increasing their affinity to the ligand-binding region over the haem binding region may decrease their ability to move towards the haem-binding region. Alternatively, decreasing their affinity to the ligand-binding region may be desired to decrease their affinity to this region compared to the haem binding region and hence increase their ability to move towards the haem binding region. If compound binding to the ligand-binding pocket is a necessary prerequisite of compound binding in the haem-binding region and its subsequent metabolism by or inhibition of 3A4, elimination of binding to the ligand-binding region may eliminate all compound metabolism by 3A4 or inhibition of 3A4. An alternative or additional approach is to modify such substrates to increase or decrease their affinity for residues of the haem-binding region. Changes of this type may be introduced in order to increase or decrease the turnover of the substrates.

Some molecules are known to be effectors or activators of 3A4 metabolism. Modification of the binding between 3A4 and such a compound would mediate metabolism of the substrate.

Thus in one embodiment, the present invention provides a method for modifying the structure of a compound in order to alter its metabolism by a P450, which method comprises:
fitting a starting compound to one or more coordinates of at least one amino acid residue of the ligand-binding region of the P450;
modifying the starting compound structure so as to increase or decrease its interaction with the ligand-binding region;
wherein said ligand-binding region is defined as including at least one, for example at least four, or all eight, of the P450 residues numbered as Phe57, Phe108, Phe213, Phe215, Phe219, Phe220, Phe241 and Phe304.

In another embodiment, the present invention provides a method for modifying the structure of a compound in order to alter its metabolism by a P450, which method comprises:
fitting a starting compound to one or more coordinates of at least one amino acid residue of the ligand-binding region of the P450;
modifying the starting compound structure so as to increase or decrease its interaction with the ligand-binding region;
wherein said ligand-binding region is defined as including at least one, such as at least two, for example such as at least five, preferably at least ten of the P450 residues of Table 6 and preferably of Table 7.

In another embodiment, the invention provides a method for modifying the structure of a compound in order to alter its metabolism by a P450 3A4, which method comprises:
fitting a starting compound to one or more coordinates of at least one amino acid residue of the haem-binding region of the P450;
modifying the starting compound structure so as to increase or decrease its interaction with the haem-binding region.

The haem binding region also optionally includes the iron ion bound to the haem molecule, and if desired, one or more of the other atoms of the haem molecule itself. In a preferred aspect of the invention, the iron ion is also included in the haem-binding region.

Desirably, in the above aspects of the invention, coordinates from at least two, preferably at least five, and more preferably at least ten amino acid residues of the P450 (including where desired the iron ion) will be used.

For the avoidance of doubt, the term “modifying” is used as defined in the preceding subsection, and once such a compound has been developed it may be synthesised and tested also as described above.

(v) Peripheral Binding Site and Use Thereof

It is well documented in the literature that CYP3A4 exhibits atypical kinetics including both homotrophic and hetereotrophic cooperativity. Cooperativity is the stimulation or inhibition of catalytic activity of one compound by either the same compound (homo cooperativity) or a different compound (hetero cooperativity). A number of residues have, by site-directed mutagenesis, been shown to play a role in CYP3A4 cooperativity but not activity, and have thus been implicated in forming part of an “effector” binding site (e.g. Leu211 (Harlow et al, J. Biol. Chem, 1997, 272:5396-5402) and Asp214 (Harlow et al, PNAS, 1998, 95:6636-6641)) which may be distinct from the active site as defined in Table 5. These two residues lie close to the progesterone molecule bound to CYP3A4 (defined by Table 3).

In one hypothesis the peripheral binding site of progesterone is an effector binding site, thus designing a compound molecule to bind at this peripheral site may increase or decrease binding of the same or different compound molecule within the active site of CYP3A4. An alternative or additional approach is to modify compounds to increase or decrease their affinity for residues of the peripheral binding region. For example, knowledge of this peripheral binding site will enable the re-design of compounds such that they do not bind to this peripheral binding site (defined herein as Phe213, Asp214, Phe219), thus designing out any cooperativity effects that may be undesirable. By altering (i.e. increasing or decreasing) a compounds affinity to the peripheral binding site compared to the active binding site as defined by Table 6 it may alter (i.e. increase or decrease) the compounds metabolism or alter the metabolism of other compounds. Thus one aspect of the invention is the modification of the binding of a compound to the residues of the peripheral binding site to modify the metabolism of the same compound or a different compound.

The binding of some CYP3A4 compounds in this peripheral binding site may be a prerequisite for them entering the CYP3A4 active site for metabolism. For example, S-warfarin has been observed to bind to hydrophobic pocket in CYP2C9 that is remote from the haem group. The S-warfarin binding site is at a distance from the haem, widely suggesting that subsequent movement of the compound would be required for hydroxylation of S-warfarin to occur. In the same way, this remote binding pocket on CYP3A4 may also serve as a “pre-binding pocket”, facilitating the physiological metabolism of certain compounds.

The structure of 3A4 with progesterone shows that the binding pocket in which progesterone binds is physically distinct from the region of the molecule in which the haem is located. In our structure the progesterone is located on the periphery of the protein, some 17 Å away from the haem iron, and not in a location favouring metabolism. Thus the progesterone molecule is located in a binding site that may represent a holding position, and ligands may have to move from this site towards the haem binding site for metabolism to occur. The movement of a ligand from this holding site towards the haem may be triggered by a protein conformational movement. Rearrangement of the Phe-cluster, which is positioned below the progesterone binding site, so as to open up the active site of 3A4, could be such a conformational movement.

Such a mechanism provides a means to modify ligands of 3A4 in order to alter their metabolism. By altering (i.e. increasing or decreasing) a ligand's affinity to the peripheral binding region compared to the haem binding region it may alter (i.e. increase or decrease) their ability to move towards the haem-binding region. For example by increasing a ligand's affinity to the peripheral binding region over the haem binding region may decrease their ability to move towards the haem-binding region. Alternatively, decreasing their affinity to the peripheral binding region may be desired to decrease their affinity to this region compared to the haem binding region and hence increase their ability to move towards the haem binding region. If compound binding to the peripheral binding pocket is a necessary prerequisite of compound binding in the haem-binding region and its subsequent metabolism by or inhibition of 3A4, elimination of binding to the peripheral binding region may eliminate all compound metabolism by 3A4 or inhibition of 3A4. An alternative or additional approach is to modify such substrates to increase or decrease their affinity for residues of the haem-binding region. Changes of this type may be introduced in order to increase or decrease the turnover of the substrates.

Thus in one embodiment, the present invention provides a method for modifying the structure of a compound in order to alter the compound's, or another compounds, metabolism by a P450, which method comprises:
fitting a starting compound to one or more coordinates of at least one amino acid residue of the peripheral binding region of the P450;
modifying the starting compound structure so as to increase or decrease its interaction with the peripheral binding region;
wherein said peripheral binding region is defined as the P450 residues numbered as: 213, 214, 219.

In one case, a compound may be designed to target the active site of CYP3A4 and then it may be possible to modify this compounds metabolism properties by coadministrating with a compound which targets this peripheral binding site. CYP3A4 metabolises endogenous compounds, including steroids such as progesterone and therefore it may be possible to “control” endogenous cooperativity by designing compounds with increased or decreased affinity to this peripheral binding site.

Thus in another embodiment, the present invention provides a method for designing the structure of a compound which binds to the peripheral binding region, in order to alter another compounds metabolism by a P450, which method comprises:
fitting a starting compound to one or more coordinates of at least one amino acid residue of the peripheral binding region of the P450;
modifying the starting compound structure so as to increase or decrease its interaction with the peripheral binding region;
wherein said peripheral binding region is defined as the P450 residues numbered as: 213, 214, 219.

The above embodiments of the invention may be performed in conjunction with the previously described embodiments wherein a compound is fitted to the ligand-binding region of P450. This may be done for example to analyse the binding of a compound to the ligand-binding region before and after modification of a structure fitted to the peripheral binding site. Alternatively, the structure fitted to the ligand-binding region may itself be modified as described above wherein a structure is also fitted to the peripheral binding site.

(vi) Fragment Linking and Growing.

The provision of the crystal structures of the invention will also allow the development of compounds which interact with the binding pocket regions of P450s (for example to act as inhibitors of a P450) based on a fragment linking or fragment growing approach.

For example, the binding of one or more molecular fragments can be determined in the protein binding pocket by X-ray crystallography. Molecular fragments are typically compounds with a molecular weight between 100 and 200 Da (Carr et al, 2002). This can then provide a starting point for medicinal chemistry to optimise the interactions using a structure-based approach. The fragments can be combined onto a template or used as the starting point for ‘growing out’ an inhibitor into other pockets of the protein (Blundell et al, 2002). The fragments can be positioned in the binding pocket of the P450 and then ‘grown’ to fill the space available, exploring the electrostatic, van der Waals or hydrogen-bonding interactions that are involved in molecular recognition. The potency of the original weakly binding fragment thus can be rapidly improved using iterative structure-based chemical synthesis.

At one or more stages in the fragment growing approach, the compound may be synthesized and tested in a biological system for its activity. This can be used to guide the further growing out of the fragment.

Where two fragment-binding regions are identified, a linked fragment approach may be based upon attempting to link the two fragments directly, or growing one or both fragments in the manner described above in order to obtain a larger, linked structure, which may have the desired properties.

Where the binding site of two or more ligands are determined they may be connected to form a potential lead compound that can be further refined using e.g. the iterative technique of Greer et al. For a virtual linked-fragment approach see Verlinde et al., J. of Computer-Aided Molecular Design, 6, (1992), 131-147, and for NMR and X-ray approaches see Shuker et al., Science, 274, (1996), 1531-1534 and Stout et al., Structure, 6, (1998), 839-848. The use of these approaches to design P450 inhibitors is made possible by the determination of the P450 structure.

(vii) Compounds of the Invention.

Where a potential modified compound has been developed by fitting a starting compound to the P450 structure of the invention and predicting from this a modified compound with an altered rate of metabolism (including a slower, faster or zero rate), the invention further includes the step of synthesizing the modified compound and testing it in an in vivo or in vitro biological system in order to determine its activity and/or the rate at which it is metabolised.

The method comprises: (a) providing 3A4 under conditions where, in the absence of modulator, the 3A4 is able to metabolise known substrates; (b) providing the compound; and (c) determining the extent to which the compound is metabolised in the presence of 3A4 or (d) determining the extent to which the compound inhibits metabolism of a known substrate of 3A4.

More preferably, in the latter steps the compound is contacted with P450 under conditions to determine its function.

For example, in the contacting step above the compound is contacted with P450 in the presence of the compound, and typically a buffer and substrate, to determine the ability of said compound to inhibit P450 or to be metabolised by P450. The substrate may be e.g. dibenzylfluorescein. So, for example, an assay mixture for P450 may be produced which comprises the compound, substrate and buffer.

In another aspect, the invention includes a compound, which is identified by the methods of the invention described above.

Following identification of such a compound, it may be manufactured and/or used in the preparation, i.e. manufacture or formulation, of a composition such as a medicament, pharmaceutical composition or drug. These may be administered to individuals.

Thus, the present invention extends in various aspects not only to a compound as provided by the invention, but also a pharmaceutical composition, medicament, drug or other composition comprising such a compound. The compositions may be used. for treatment (which may include preventative treatment) of disease such as cancer. Such a treatment may comprise administration of such a composition to a patient, e.g. for treatment of disease; the use of such an inhibitor in the manufacture of a composition for administration, e.g. for treatment of disease; and a method of making a pharmaceutical composition comprising admixing such an inhibitor with a pharmaceutically acceptable excipient, vehicle or carrier, and optionally other ingredients.

Thus a further aspect of the present invention provides a method for preparing a medicament, pharmaceutical composition or drug, the method comprising:

(a) identifying or modifying a compound by a method of any one of the other aspects of the invention disclosed herein; (b) optimising the structure of the molecule; and (c) preparing a medicament, pharmaceutical composition or drug containing the optimised compound.

The above-described processes of the invention may be iterated in that the modified compound may itself be the basis for further compound design.

By “optimising the structure” we mean e.g. adding molecular scaffolding, adding or varying functional groups, or connecting the molecule with other molecules (e.g. using a fragment linking approach) such that the chemical structure of the modulator molecule is changed while its original modulating functionality is maintained or enhanced. Such optimisation is regularly undertaken during drug development programmes to e.g. enhance potency, promote pharmacological acceptability, increase chemical stability etc. of lead compounds.

Modification will be those conventional in the art known to the skilled medicinal chemist, and will include, for example, substitutions or removal of groups containing residues which interact with the amino acid side chain groups of a P450 structure of the invention. For example, the replacements may include the addition or removal of groups in order to decrease or increase the charge of a group in a test compound, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting compound and its activity.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The invention is illustrated by the following examples:

### EXAMPLE

Cloning of 3A4

3A4 corresponding to M18907 (GI_{—}181373) was cloned from human liver library (Origene Technologies, Inc.).

PCR carried out as recommended by the manufacturer:

| | |
|---|---|
| Liver library | 2.0 μl |
| 10 × PCR buffer (−Mg²⁺) | 2.5 μl |
| 10 mM dNTPs | 0.5 μl |
| 10 mM MgSO₄ | 2.5 μl |
| Water | 11.0 μl |
| Primer 1 (@10 pmol/μl) | 3.0 μl |
| Primer 2 (@10 pmol/μl) | 3.0 μl |

Primer 1 is complementary to the 5′ end of the full length 3A4 cDNA. Primer 2 is complementary to the 3′ end of the cDNA and adds a four histidine tag onto the C-terminus of the 3A4 protein.

Heat to 94° C., add 0.5 μl (1 Unit) Vent polymerase

35 cycles as follows:

| | |
|---|---|
| 94° C. | 30 seconds |
| 65° C. | 60 seconds |
| 72° C. | 60 seconds |

1 cycle of 72° C. for 5 minutes.

Following the addition of 1 μl (2.5 Units) Taq polymerase and incubation at 72° C. for 10 minutes, 1 μl of product was used in a TOPO cloning reaction (vector pCR4TOPO, Invitrogen). The cloning reaction was used to transform E. coli XL1-blue and positive clones identified by NdeI/SalI restriction digestion of purified plasmids. Positive clones were sequenced fully on both strands and the NdeI/SalI insert subcloned into pET20b to yield the template clone. This clone was used as the template in subsequent PCR reactions.

N-Terminal Truncation of 3A4

The expression vector pCWOri+, provided by Prof. F. W. Dahlquist, University of Oregon, Eugene, Oreg., USA, was used to express the truncated human cytochrome P450 in the E. coli strain XL1 Blue (Stratagene). Full-length CDNA encoding cytochrome P450 3A4 isolated above was used as a template for PCR amplification, engineering the 5′ terminus and insertion of a four Histidine tag at the C-terminus.

N-terminal truncation of 3A4 was carried out by PCR as outlined below, to generate the published NF10 N-terminal truncation described by Gillam (Gillam et al, Arch. Biochem. Biophys. Vol. 305, 123-131, 1993).

| | | |
|---|---|---|
| Template | ˜5 | ng |
| 10 × PCR buffer (+Mg²⁺) | 5.0 | μl |
| 10 mM dNTPs | 1.0 | μl |
| Water | 42.0 | μl |
| Primer 2 (@100 pmol/μl) | 0.5 | μl |
| Primer 3 (@100 pmol/μl) | 0.5 | μl |
| Vent polymerase (2 U/μl) | 0.5 | μl |

25 cycles of:

| | |
|---|---|
| 94° C. | 30 seconds |
| 65° C. | 60 seconds |
| 72° C. | 60 seconds |

1 cycle of 72° C. for 5 minutes.

Following the addition of 1 μl (2.5 units) Taq polymerase and incubation at 72° C. for 10 minutes, 1 μl of product was used in a TOPO cloning reaction (vector pCR4TOPO, Invitrogen). The cloning reaction was used to transform E. coli XL1-blue and positive clones identified by NdeI/SalI restriction digestion of purified plasmids. Positive clones were sequenced fully and the NdeI/SalI insert subcloned into pCWori+ to yield clone p3A4. This clone was used for protein expression.

Primer 1
5′-GGAATTCATATGGCTCTCATCCCAGACTTGGCC-3′
Primer 2
5′-TGCGGTCGACTCAATGGTGATGGTGGGCTCCACTTACGGTGCCATC
C-3′
Primer 3
5′-TTAACATATGGCATATGGTACTCATTCACATGGTCTGTTTAAAAAAC
TGGGAATTCCAGGGCCCACACC-3′

Bacterial Expression

A single ampicillin resistant colony of XL1 blue cells was grown overnight at 37° C. in Terrific Broth (TB) with shaking to near saturation and used to inoculate fresh TB media. Bacteria were grown to an OD600 nm=0.5 in 1 litre of TB broth containing 100 μg/ml of ampicillin at 37° C. at 185 rpm in 2 litre flask. The haem precursor delta aminolevulinic acid (80 mg/l) was added 30 min prior to induction with 1 mM isopropyl-β-D-thiogalactopyranoside (IPTG) and the temperature lowered to 25° C. The bacterial culture was continued under agitation at 25° C. for 48 hours.

Protein Purification 1A

Cells expressing 3A4 grown as described above were pelleted at 10000 g for 10 min and resuspended in a buffer containing 500 mM KPi, pH 7.4, 20% glycerol, 10 mM mercaptoethanol, 0.1% (v/v) of protease inhibitor cocktail (Calbiochem), 10 mM imidazole, 40 U/ml DNase 1 and 5 mM MgSO₄.

The cells were lysed by passing twice through a Constant Systems Cell Homogeniser at 10000 psi. The cell debris was then removed by centrifugation at 22000×g at 4° C. for 30 min.

Detergent IGEPAL CA630 (Sigma) was added dropwise from a 10% stock solution to the lysate at a final concentration of 0.3% (v/v) and the lysate was incubated with previously washed NiNTA resin (Qiagen) overnight at 4° C., using agitation. The protein bound-NiNTA resin was
pelleted by centrifugation at 2000 g for 2 min at 4° C. The resin was washed with 20 resin volumes of 500 mM KPi, pH 7.4, 20% glycerol, 10 mM mercaptoethanol, 10 mM imidazole, 1:1000 dilution of protease inhibitor cocktail, 0.3% (v/v) IGEPAL CA630 and the resin pelleted by centrifugation at 2000×g for 2 min at 4° C. The resin was then washed with 10 resin volumes of 500 mM KPi, pH 7.4, 20% glycerol, 10 mM mercaptoethanol, 20 mM imidazole, 0.1% (v/v) protease inhibitors, 0.3% IGEPAL CA630 and the resin recovered by centrifugation as described above.

The resin was packed into a column at 4° C. and the cytochrome P450 eluted with 500 mM KPi, pH 7.4, 20% glycerol, 10 mM mercaptoethanol, 300 mM imidazole, 0.1% (v/v) of protease inhibitor cocktail, 0.3% (v/v) IGEPAL CA630.

The cytochrome P450 obtained from the NiNTA column was quickly desalted into 10 mM KPi, pH 7.4, 20% glycerol, 2.0 mM DTT, 1 mM EDTA using a HiPrep 26/10 desalting column (Pharmacia), at a flow rate of 5 ml/min.

The desalted cytochrome P450 was directly applied to a CM Sepharose column (Pharmacia), previously equilibrated with 10 mM KPi, pH 7.4, 20% glycerol, 2.0 mM DTT, 1 mM EDTA. The following step elution was applied: wash with 20 column volumes of 10 mM KPi, pH 7.4, 20% glycerol, 2.0 mM DTT, 1 mM EDTA, wash with the above buffer with 75 mM KCl in order to remove any trace of detergent, then eluted with the above buffer with KCl concentration increased to 500 mM.

The protein was concentrated up to 40 mg/ml using a microconcentrator for crystallization assays.

Protein Characterization

The quality of the final preparation was evaluated by:

(a) SDS polyacrylamide gel electrophoresis: This was performed using commercial gels (Nugen) followed by CBB staining according to the manufacturer's instructions. The purity as estimated by scanning a digital image of a gel was estimated to be at least 95%.

(b) Mass Spectroscopy This was performed using a Bruker “BioTOF” electrospray time of flight instrument. Samples were either diluted by a factor of 1000 straight from storage buffer into methanol/water/formic acid (50:48:2 v/v/v), or subjected to reverse phase HPLC separation using a C4 column.

Calibration was achieved using Bombesin and angiotensin I using the 2+ and 1+ charged states. Data were acquired between 200 and 2000 m/z range and were subsequently processed using Bruker's X-mass program. Mass accuracy was typically below 1 in 10 000.

Mass spec of 3A4: 55281 Da(observed)
55278 Da (predicted minus N-terminal methionine)
   Crystallization 1A

Crystals of the 3A4 were grown using the hanging drop vapor diffusion method. Protein at 40 mg/ml in 10 mM Kpi pH 7.4, 0.5 M KCl, 2 mM DTT, 1 mM EDTA. 20% glycerol, was mixed in a 1:1 ratio, using 0.5 ul drops, with a reservoir solution. The crystals of 3A4 grew over a reservoir solution containing 0.1 M HEPES pH 7.5, 0.2 M sodium chloride, 30% PEG 400.

Alterative conditions are listed below:

0.1 M HEPES pH 7.5, 0.2 M sodium chloride, 30% PEG 400

0.05 M HEPES pH 7.5, 0.2 M sodium chloride, 35% PEG 400

0.05 M HEPES pH 7.5, 0.2 M sodium chloride, 30% PEG 400

0.15 M Imidazole-HCl pH 8, 10% 2-propanol

0.1 M 2-(N-cyclohexylamino)ethanesulfonic acid (CHES) pH 9.5, 30% PEG 400

0.15 M Hepes—Na pH 7.5, 5% IPA, 10% Peg 4000

0.1 M phosphate-citrate pH 4.2, 1.6 M NaH2PO4/0.4M K2HPO4

0.1 M citrate pH 5.5, 0.2 sodium chloride, 1.0 M Ammonium phosphate

0.2 M Lithium chloride, 20% PEG 3350

0.2 M Potassium chloride, 20% PEG 3350

0.2 M Sodium formate, 20% PEG 3350

0.2 M Potassium formate, 20% PEG 3350

0.2 M Ammonium formate, 20% PEG 3350

0.2 M Lithium acetate, 20% PEG 3350

0.2 M Potassium chloride, 20% PEG 3350

0.2 M Sodium formate, 20% PEG 3350

0.2 M Lithium acetate, 20% PEG 3350

0.2 M Sodium acetate, 20% PEG 3350

0.2 M Potassium acetate, 20% PEG 3350

0.2 M Ammonium acetate, 20% PEG 3350

0.1 M HEPES pH 7.5, 0.2 M sodium chloride, 30% PEG 400

0.1 M HEPES pH 7.5, 5% Iso-Propanol, 10% PEG 4000

200 mM K Acetate, 25% peg 3350

200 mM K Acetate, 25% peg 3350

300 mM Na acetate, 25% peg 3350

200 mM Sodium formate, 25% PEG 3350

0.300 M Lithium acetate, 25.0% PEG 3350

0.100 M Imidazole-HCl pH 8, 10% 2-propanol

0.150 M Imidazole-HCl pH 8, 10% 2-propanol

Crystals formed within 1-7 days at 25° C., and were rod shaped in morphology.

The approximate cell dimensions of the crystals were a=77 Å, b=99 Å, c=129 Å, β=90°. The space group is I222.

The crystals were flash frozen in liquid nitrogen, using 80% reservoir solution, 20% ethylene glycol as a cryoprotectant.

Crystals of 3A4 were also grown over a reservoir solution containing:

0.15M HEPES pH7.5, 5% IPA, 10% PEG 4000.

Crystals were obtained with unit cell C2: a=152 Å, b=101 Å, c=78 Å, α=90°, β=120°, γ=90°. The invention thus provides crystal of 3A4 having this space group and unit cell dimensions, the dimensions a, b and c and β varying independently by +/−5%.

The crystal form obtained belonging to spacegroup I222, with cell dimensions 77 Å, 99 Å, 129 Å, 90°, 90°, 90° contains one copy in the asymmetric unit. As is true with many crystal forms, data from this crystal can be processed in the lower symmetry of C2, with cell dimensions 152 Å, 101 Å, 78 Å, 90°, 120°, 90° and two copies in the asymmetric unit. The relationship between the two is that in the C2 classification, the two molecules in the asymmetric unit are related by a 180° rotation, which in the I222 classification is treated as a crystallographic (and not a non-crystallographic) rotation. The difference between a crystallographic two-fold and a non-crystallographic two-fold is that in the former the two molecules have to be identical, while in the latter the two molecules can adopt different conformations. At lower resolution two molecules may appear identical, but with the addition of higher resolution data, differences may become apparent, and hence the crystallographic symmetry may break down.

In summary the invention includes a crystal of 3A4 having a space group I222 and unit cell size a=77 Å, b=99 Å, c=129 Å, β=90°; or having a space group C2 and unit cell size a=152 Å, b=101 Å, c=78 Å, α=90°, β=120°, γ=90°. Those of skill in the art will recognise that the cell dimensions of the crystal may vary by 5%, though preferably by 1-2 Å, upon repeat crystallization, and such variation resides within the spirit and scope of the invention.

Protein Purification (1B)

The cells were pelleted at 10000g for 10 min and resuspended in a buffer containing 500 mM KPi, pH 7.4, 20% glycerol (v/v), 10 mM mercaptoethanol, 0.1% (v/v) of protease inhibitor cocktail 3 (Calbiochem), 10 mM imidazole, 40 U/ml DNase 1 and 5 mM MgSO₄.

Passing twice through a Constant Systems Cell Homogeniser at 10000 psi lysed the cells. The cell debris was then removed by centrifugation at 22000×g at 4° C. for 30 min.

Detergent IGEPAL CA630 (Sigma) was added dropwise from a 10% stock solution to the lysate at a final concentration of 0.3% (v/v) and the lysate was incubated with previously washed NiNTA resin (Qiagen) overnight at 4° C., using agitation. The protein bound-NiNTA resin was pelleted by centrifugation at 2000 g for 5 min at 4° C. The resin was washed with 20 resin volumes of 500 mM KPi, pH 7.4, 20% glycerol, 10 mM mercaptoethanol, 10 mM imidazole, 0.1% (v/v) of protease inhibitor cocktail, 0.3% (v/v) IGEPAL CA630 and the resin pelleted by centrifugation at 2000 g for 5 min at 4° C. The resin was then washed with 10 resin volumes of 500 mM KPi, pH 7.4, 20% glycerol, 10 mM mercaptoethanol, 20 mM imidazole, 0.1% (v/v) protease inhibitors, 0.3% IGEPAL CA630 and the resin recovered by centrifugation as described above.

The resin was packed into a column at room temperature and the cytochrome P450 eluted with cold 500 mM KPi, pH 7.4, 20% glycerol, 10 mM mercaptoethanol, 300 mM imidazole, 0.1% (v/v) of protease inhibitor cocktail, 0.3% (v/v) IGEPAL CA630.

The cytochrome P450 obtained from the NiNTA column was quickly desalted into 20 mM KPi, pH 7.2, 20% glycerol, 2.0 mM DTT, 1 mM EDTA using a HiPrep 26/10 desalting column (Pharmacia), at a flow rate of 5 ml/min on a Akta FPLC system (Pharmacia). A watch UV command (280 nm) of greater than 750 mAu was then used to divert the desalted P450 from the HiPrep 26/10 desalting column onto a CM Sepharose column (Pharmacia), previously equilibrated with 20 mM KPi, pH 7.2, 20% glycerol, 2.0 mM DTT, 1 mM EDTA for final purification. The peak divert was ended when the mAu fell below 750 mAu. The following step elution was then applied to the CM Sepharose column: wash with 10 column volumes of 20 mM KPi, pH 7.2, 20% glycerol, 2.0 mM DTT, 1 mM EDTA, followed by a wash with 6 column volumes with the above buffer with 75 mM KCl added in order to remove any trace of detergent, then eluted with the above buffer with KCl concentration increased to 500 mM.

The protein was concentrated up to 40 mg/ml using a microconcentrator for crystallization trials.

Crystallization (1B)

Crystals of the 3A4 were grown using the hanging drop vapor diffusion method. Protein at 37.4 mg/ml in 20 mM Kpi pH 7.2, 0.5 M KCl, 2 mM DTT, 1 mM EDTA, 20% glycerol, was mixed in a 1:1 ratio, using 0.5 ul drops, with a reservoir solution. The crystals of 3A4 grew over a reservoir solution containing 0.15 M HEPES pH 7.5, 2.5% IPA, 10% PEG 4000.

Crystals formed within 1-7 days at 25° C., and were rod shaped in morphology.

The crystals were flash frozen in liquid nitrogen, using crystallisation solution supplemented with 15% glycerol as a cryoprotectant.

Dataset Collection (1)

A native dataset was collected at the ESRF beamline 14.2 to a resolution of 2.7 Å, from a crystal produced using the protocol above in Protein purification (1B) and Crystallisation (1B).

The cell dimensions of the crystals were a=77.85 Å, b=99.71 Å, c=132.74 Å, α=β=γ=90°. The space group was I222.

A total of 100 one degree oscillation images were collected, processed with MOSFLM (Leslie, A. G. W. (1992). In Joint CCP4 and EESF-EACMB Newsletter on Protein Crystallography, vol. 26, Warrington, Daresbury Laboratory), scaled using SCALA (CCP4—Collaborative Computational Project 4. (1994) The CCP4 Suite: Programs for Protein Crystallography. Acta Crystallographica D50, 760-763) and reduced using the CCP4 suite of programs.

Protein Purification (2)

The cells were pelleted at 10000g for 10 min and resuspended in a buffer containing 500 mM KPi, pH 7.4, 20% glycerol, 10 mM mercaptoethanol, 0.1% (v/v) of protease inhibitor cocktail 3 (Calbiochem), 10 mM imidazole, 40 U/ml DNase 1 and 5 mM MgSO₄.

Passing twice through a Constant Systems Cell Homogeniser at 10000 psi lysed the cells. The cell debris was then removed by centrifugation at 22000g at 4° C. for 30 min.

Detergent IGEPAL CA630 (Sigma) was added dropwise from a 10% stock solution to the lysate at a final concentration of 0.3% (v/v) and the lysate was incubated with previously washed NiNTA resin (Qiagen) overnight at 4° C., using agitation. The protein bound-NiNTA resin was pelleted by centrifugation at 2000g for 5 min at 4° C. The resin was washed with 20 resin volumes of 500 mM KPi, pH 7.4, 20% glycerol, 10 mM mercaptoethanol, 10 mM imidazole, 0.1% (v/v) of protease inhibitor cocktail, 0.3% (v/v) IGEPAL CA630 and the resin pelleted by centrifugation at 2000g for 5 min at 4° C. The resin was then washed with 10 resin volumes of 500 mM KPi, pH 7.4, 20% glycerol, 10 mM mercaptoethanol, 20 mM imidazole, 0.1% (v/v) protease inhibitors, 0.3% IGEPAL CA630 and the resin recovered by centrifugation as described above.

The resin was packed into a column at room temperature and the cytochrome P450 eluted with cold 500 mM KPi, pH 7.4, 20% glycerol, 10 mM mercaptoethanol, 300 mM imidazole, 0.1% (v/v) of protease inhibitor cocktail, 0.3% (v/v) IGEPAL CA630.

The cytochrome P450 obtained from the NiNTA column was quickly desalted into 10 mM KPi, pH 7.2, 20% glycerol, 2.0 mM DTT, 1 mM EDTA, 10 mM K₂SO₄ using a HiPrep 26/10 desalting column (Pharmacia), at a flow rate of 5 ml/min.

The desalted cytochrome P450 was directly applied to a CM Sepharose column (Pharmacia) previously equilibrated with 10 mM KPi, pH 7.2, 20% glycerol, 2.0 mM DTT, 1 mM EDTA, 10 mM K₂SO₄. The following step elution was applied: wash with 20 column volumes of 10 mM KPi, pH 7.2, 20% glycerol, 2.0 mM DTT, 1 mM EDTA, 10 mM K₂SO₄ followed by a wash with 20 column volumes of the above buffer with 75 mM KCl in order to remove any trace of detergent, then eluted with the above buffer with KCl concentration increased to 500 mM.

The protein was concentrated up to 20 mg/ml using a microconcentrator for crystallization assays.

Crystallization (2)

Crystals of the 3A4 were grown using the hanging drop vapor diffusion method. Protein at 18.5 mg/ml in 10 mM Kpi pH 7.2, 0.5 M KCl, 2 mM DTT, 1 mM EDTA, 20% glycerol, 10 mM K2SO4 was mixed in a 1:1 ratio, using 0.5 ul drops, with a reservoir solution. The crystals of 3A4 grew over a reservoir solution containing 0.1 M HEPES pH 7.2, 5% IPA, 10% PEG 4000. The crystal was frozen using the crystallization solution supplemented by glycerol to 33%.

Crystals formed within 1-7 days at 25° C., and were rod shaped in morphology.

Dataset Collection (2)

A native dataset was collected at the ESRF beamline 14.2 to a resolution of 2.8 Å, from a crystal produced using the protocol above in Protein purification (2) and Crystallisation (2).

The approximate cell dimensions of the crystals were a=77.32 Å, b=100.37 Å, c=132.72 Å, α=β=γ=90°. The space group was I222.

A total of eighty one degree oscillation images were collected, processed with MOSFLM (Leslie, A. G. W. (1992). In Joint CCP4 and EESF-EACMB Newsletter on Protein Crystallography, vol. 26, Warrington, Daresbury Laboratory), scaled using SCALA (CCP4—Collaborative Computational Project 4. (1994) The CCP4 Suite: Programs for Protein Crystallography, Acta Crystallographica D50, 760-763) and reduced using the CCP4 suite of programs.

Protein Purification (3)

The cells were pelleted at 10000g for 10 min and resuspended in a buffer containing 500 mM KPi, pH 7.4, 20% glycerol, 10 mM mercaptoethanol, 0.1% (v/v) of protease inhibitor cocktail 3 (Calbiochem), 10 mM imidazole, 40 U/ml DNase 1 and 5 mM MgSO₄.

Passing twice through a Constant Systems Cell Homogeniser at 10000 psi lysed the cells. The cell debris was then removed by centrifugation at 22000×g at 4° C. for 30 min.

Detergent IGEPAL CA630 (Sigma) was added dropwise from a 10% stock solution to the lysate at a final concentration of 0.3% (v/v) and the lysate was incubated with previously washed NiNTA resin (Qiagen) overnight at 4° C., using agitation. The protein bound-NiNTA resin was pelleted by centrifugation, 2000g for 5 min at 4° C. The resin was washed with 20 resin volumes of 500 mM KPi, pH 7.4, 20% glycerol, 10 mM mercaptoethanol, 10 mM imidazole, 0.1% (v/v) of protease inhibitor cocktail, 0.3% (v/v) IGEPAL CA630 and the resin pelleted by centrifugation at 2000g for 5 min at 4° C. The resin was then washed with 10 resin volumes of 500 mM KPi, pH 7.4, 20% glycerol, 10 mM mercaptoethanol, 20 mM imidazole, 0.1% (v/v) protease inhibitors, 0.3% IGEPAL CA630 and the resin recovered by centrifugation as described above.

The resin was packed into a column at room temperature and the cytochrome P450 eluted with cold 500 mM KPi, pH 7.4, 20% glycerol, 10 mM mercaptoethanol, 300 mM imidazole, 0.1% (v/v) of protease inhibitor cocktail, 0.3% (v/v) IGEPAL CA630.

The cytochrome P450 obtained from the NiNTA column was quickly desalted into 10 mM KPi, pH 7.2, 20% glycerol, 2.0 mM DTT, 1 mM EDTA using a HiPrep 26/10 desalting column (Pharmacia), at a flow rate of 5 ml/min.

The desalted cytochrome P450 was directly applied to a CM Sepharose column (Pharmacia) previously equilibrated with 10 mM KPi, pH 7.2, 20% glycerol, 2.0 mM DTT, 1 mM EDTA. The following step elution was applied: wash with 20 column volumes of 10 mM KPi, pH 7.2, 20% glycerol, 2.0 mM DTT, 1 mM EDTA, followed by a wash with 20 column volumes of the above buffer with 75 mM KCl in order to remove any trace of detergent, then eluted with the above buffer with KCl concentration increased to 500 mM.

The concentrated sample (200 μL, 7.9 mg protein) was then gel filtered using a Superdex 200 HR10/30 column (Pharmacia) in 10 mM KPi, pH7.2, 20% glycerol, 1 mM EDTA, 2 mM DTT, 500 mM KCl at a flow rate of 0.4 ml/min. Fractions of 0.5 ml were collected. Three peaks of protein were collected, of these the first (elution volume, Ve=8.64 ml) represented aggregated protein that had been excluded by the void volume, Vo (Vo=8.66 ml) of the column, the second peak (Ve=12.4 ml) was the largest and represented the P450, and the third and smallest peak (Ve=15.49 ml) was low molecular weight protein contaminants.

The P450 peak was then pooled and concentrated up to 40 mg/ml using a microconcentrator for crystallization trials. 3A4 can alternatively be purified by gel filtration chromatography, by passage down a 26/60 Superdex 200 column equilibrated in 10 mM K Pi pH 7.2, 20% glycerol, 0.5M KCl, 2 mM DTT run at 1.5 mg/ml, to improve homogeneity for crystallisation.

Crystallization (3)

Crystals of the 3A4 were grown using the hanging drop vapor diffusion method. Protein at 36 mg/ml in 10 mM Kpi pH 7.2, 0.5 M KCl, 2 mM DTT, 1 mM EDTA, 20% glycerol, was mixed in a 1:1 ratio, using 0.5 μl drops, with a reservoir solution. The crystals of 3A4 grew over a reservoir solution containing 0.1 M HEPES pH 7.5, 0.025 M sodium chloride, 7.5% IPA, 10% PEG 4000.

The crystals formed over a number of days at 25° C., and were rod shaped in morphology.

The crystals were transferred to a cryo-solution consisting of 0.1 M HEPES pH 7.5, 0.25 M KCl, 15% PEG 4000 and 20% glycerol and then frozen in liquid nitrogen prior to data collection.

Dataset Collection (3)

Data was collected from a single crystal, produced using the protocol above in Protein purification (3) and Crystallisation (3), at beamline ID29 at the European Synchrotron Radiation Facility to a resolution of 2.8 Å. An energy scan was taken from the crystal prior to data collection to determine the precise energy at which the haem iron provided a detectable signal. The energy scan indicated the peak energy to be 7.126 KeV (corresponding to a wavelength of 1.7398 Å), and a suitable point of inflection wavelength to be 7.123 KeV (corresponding to a wavelength of 1.7406 Å).

The approximate cell dimensions of the crystals were a=77.94 Å, b=100.91 Å, c=131.00 Å, α=β=γ=90°. The space group was I222.

Two datasets were collected from a single crystal, one at a wavelength of 1.7398 Å (peak dataset) to a resolution of 2.8 Å and the second at a wavelength of 1.7406 Å (inflection dataset) to a resolution of 3.1 Å. A total of 180° of data were collected at each wavelength to ensure that the data were redundant. The data were processed using MOSFLM (Leslie, A. G. W. (1992). In Joint CCP4 and EESF-EACMB Newsletter on Protein Crystallography, vol. 26, Warrington, Daresbury Laboratory), scaled using SCALA (CCP4 computing package (Collaborative Computational Project 4. The CCP4 Suite: Programs for Protein Crystallography, Acta Crystallographica, D50, (1994), 760-763) and further reduced using the CCP4 suite of programs.

MAD Structure Determination

The location of the iron atom within the unit cell was determined by visual inspection of the three Harker sections of the anomalous difference Patterson map calculated using the peak anomalous data by the program FFT (part of the CCP4 suite).

The refined parameters of the iron atom used to generated phases are as follows: x=23.255, y=23.237, z=10.742, occupancy=0.92, temperature factor=69.45. These refined parameters were obtained using the program SHARP, by refinement against the experimental data obtained from the crystal. These atom parameters were then used within SHARP to generate phases for 3A4. These phases can then be modified by density modification procedures. The phases from SHARP were solvent flattened using SOLOMON/DM as available through the SHARP program.

We choose to refine the iron atom parameters within SHARP, generate phases within SHARP and then perform density modification using SOLOMON and DM as implemented through SHARP. It however would be possible to generate phases using the heavy atom parameters given above and to solvent flatten the resulting phases using alternative programs (for example using the CCP4 program MLPHARE ((Z. Otwinowski: Daresbury Study Weekend proceedings, 1991) to generate the phases and the CCP4 program DM (K. Cowtan (1994), Joint CCP4 and ESF-EACBM Newsletter on Protein Crystallography, 31, p34-38).

The generation of such phases (unflattened or solvent flattened) is reliant on determining accurate parameters that describe the heavy or anomalous atom (in this case the iron of the haem), as are given above.

This assignment of the iron position was consistent with the given space group I222 and not with the alternative choice I2₁2₁2₁. Both datasets together with the spacegroup I222 were giving to the program autoSHARP (Vonrhein, C. & Bricogne, G., autoSHARP (2003) Version 3.0.15. An Automated Structure Determination System. Global Phasing Ltd, Cambridge, UK) that automatically determined the position and handedness of the heavy atom substructure solution, resulting in a set of phases after density modification. The resulting density modified phases were used as phase restraints during further refinement of the heavy atom model in SHARP (La Fortelle, E. de and Bricogne, G. (1997). Maximum-likelihood heavy-atom parameter refinement for multiple isomorphous replacement and multiwavelength anomalous diffraction methods. Methods in Enzymology 276, 472-494) to give a set of phases (phase set I). In a similar heavy atom refinement and phasing experiment, using the peak wavelength alone, a set of phases (phase set II) was obtained.

The resulting phases (phase set I) were used in phased molecular replacement as implemented in MOLREP (A. Vagin, A. Teplyakov, J. Appl. Cryst. (1997) 30, 1022-1025, part of the CCP4 suite) and using 2C5 with the haem excluded (pdbent 1DT6) as a search model together with the sequence of SEQ ID 2. This gave an unambiguous solution where the haem moiety was consistent with the iron position obtained through inspection of the Harker sections.

The oriented and positioned model (based on 1DT6 and the sequence of SEQ ID 2), model-A, was used together with the phase set II phases in density modification as implement in SOLOMON (Abrahams J. P. and Leslie A. G. W., Acta Crystallographica D52, (1996), 30-42) through the SHARP program package.

The resulting electron density map showed clear structural features. When comparing the electron density with the molecular replacement solution, the secondary structure of P450 was apparent, although structural elements were clearly slightly displaced from their location in the 2C5 search model. The haem group, missing from the molecular replacement model, has clearly defined planar electron density.

Protein Characterization

The final quality of each of the protein preparations was evaluated by:

(a) SDS Polyacrylamide Gel Electrophoresis

This was performed using commercial gels (Nugen) followed by coomassie brilliant blue (CBB) staining according to the manufacturer's instructions. The purity as estimated by scanning a digital image of a gel was estimated to be at least 95%.

(b) Mass Spectroscopy

Mass spectrometry was performed using a Bruker BioTOF II electrospray time of flight instrument. Samples were either diluted by a factor of 1000 straight from storage buffer into methanol/water/formic acid (50:48:2 v/v/v), or subjected to a reverse phase separation using a C4 Millipore ‘zip-tip’ or a C4 HPLC column, before being diluted into methanol/water/formic acid.

Calibration was achieved by measurement of the 2+ and 1+ charge states of a peptide mixture containing Bombesin and angiotensin I or by using the multiple charge states of Horse Myoglobin. Data were acquired in the m/z range 200 to 2000 and were subsequently processed using Bruker's X-mass program. Mass accuracy was expected to be better than 1 in 10 000 (100 ppm).

Mass spec of 3A4: 55279.43 Da (observed) 55277.81 Da (predicted for protein minus the N-terminus Methionine)

(c) Functionality Assays

Activity assays on 3A4 were performed using dibenzylfluorescein (Gentest), which is dealkylated to the fluorescein ester, as a fluorescent substrate.

Assays were carried out in 96-well half-area black, Costar plates in a final assay volume of 50 μl. The reaction rates were monitored for 1 hour at room temperature on a Fluoroscan Ascent FL Instruments (Labsystem) platereader with excitation and emission wavelengths of 485 nm and 538 nm respectively. Reaction rates were measured using Prizm (GraphPad) software

Reaction mixtures were composed of 300 nM of 3A4 enzyme incubated with 2 units/ml purified human oxidoreductase, 2.8 μM dibenzylfluorescein and a regeneration system composed of 140 μM NADP⁺, 400 μM glucose-6-phosphate and 2.8 units/ml glucose-6-phosphate dehydrogenase in 100 mM potassium phosphate pH 7.8, 1 mM MgCl₂.

3A4 Structure Determination.

Using the electron density map obtained in the previous examples, a model of 3A4 was built using the graphical program O (Jones, T. A., Zou, J. Y., Cowan, S. W., and Kjeldgaard (1991) Acta Cryst A47, 110-119). This model was then refined to 2.8 Å resolution against the peak wavelength dataset from the iron MAD experiment using the programs CNX (Brünger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., and Warren, G. L. (1998) Acta Cryst D54, 905-921) and Refmac (Murshudov, G. N., Vagin, A. A., and Dodson, E. J. (1997) Acta Cryst. D50, 760-763). The refinement statistics in Table 8 are of the structure given in Table 1. The structure includes 29 ordered water molecules.

TABLE 8
Refinement statistics of the 3A4 crystal structure:

| | |
|---|---|
| Resolution | 2.8 Å |
| R factor | 24.36% |
| Free R factor (5% of data) | 27.38% |
| r.m.s.d. bonds | 0.0083 Å |
| r.m.s.d. angles | 1.904° |
| Average B factor (all atoms) | 64 Å² |

Co-Crystal with Metyrapone.

3A4 protein produced essentially as described in “Protein purification (3)” above was used to obtain co-crystals with metyrapone, apart from replacing the CM-Sepharose column with a CM-fast flow column. Co-crystals of metyrapone were generated using crystallisation conditions of 0.1M Hepes pH 7.5, 0.25M sodium chloride, 5% MPD, 50 mM Calcium chloride, 10% (w/v) PEG 4000, and soaking the crystals in 0.5mM metyrapone for 4 hours. Crystals were frozen using 0.05 M Hepes pH 7.5, 0.25 M sodium chloride, 5% (w/v) PEG 4000, 13% (w/v) 2-methyl-2,4-pentanediol, and 10% (w/v) glycerol and X-ray data were collected in-house to 2.7 Å resolution. The metyrapone-binding mode observed in CYP3A4 was identified in the initial electron density difference maps. Refinement of the compound in alternative binding conformation observed for P450cam resulted in positive and negative difference density in the Fo-Fc electron density maps. We currently cannot rule that both binding modes are present within the crystalline CYP3A4, but the conformation described appears to be the dominant one. The Fe—N bond distance was loosely restrained to2-2-2.3 Å during the refinement.

The coordinates of the co-rystal are set out in Table 2.

Co-Crystal with Progesterone.

3A4 protein produced essentially as described in “Protein purification (3)” above was used to obtain co-crystals with progesterone, apart from replacing the CM-Sepharose column with a CM-fast flow column. Co-crystals of progesterone were obtained by co-crystallisation using 0.5 mM progesterone in 2.5% ethanol and crystallisation conditions of 0.1M HEPES pH 7.5, 0.25M potassium chloride, 12% (w/v) PEG 4000, 5% (w/v) MPD, 25 mM calcium chloride.

Crystals were frozen using using 0.05 M Hepes pH 7.5, 0.25M sodium chloride, 5% (w/v) PEG 4000, 13% (w/v) 2-methyl-2,4-pentanediol, and 10% (w/v) glycerol. X-ray data were collection on beam line 14.1 at the ESRF to 2.65 Å resolution. In both complex structures a small rearrangement occurs away from the active site; the ligand-free and complexed structures diverge at residue Val95 towards the end of helix B, at the beginning of the long B-B′ loop region, but converge again at residue Phe102.

The coordinates of the co-crystal are set out in Table 3.

X-Ray Data Collection and Sefinement statistics.

The X-ray collection and refinement statistics for the crystals whose structures are set out in Tables 1-3 are summarised in the following Table:

| | | | | | Metyrapone | |
|---|---|---|---|---|---|---|
| | Apo CYP3A4 | | Progesterone | | CYP3A4 | |
| | structure | | CYP3A4 structure | | structure | |
| Spacegroup | I222 | | I222 | | I222 | |
| Cell dimensions | a = 77.94 Å, | | a = 77.41, | | a = 77.87, | |
| | b = 100.91 Å | | b = 101.51 Å | | b = 102.04 Å | |
| | c = 131.00 Å | | c = 128.66 Å | | c = 130.41 Å | |
| No. of reflections | 156995 | | 127063 | | 137770 | |
| No. of unique reflections | 12465 | | 14651 | | 13760 | |
| Resolution | 2.8 | Å | 79-2.65 | Å (2.79-2.65) | 40.18-2.70 | (2.85-2.7) |
| R merge¹ (%) | 7.2 | (62.6) | 4.5 | (32.8) | 4.0 | (38.1) |
| Completeness (%) | 95.6 | (74.5) | 97.8 | (97.8) | 94.8 | (97.2) |
| Multiplicity | 6.4 | (4.8) | 3.6 | (3.4) | 2.8 | (2.7) |
| I/SigmaI | 6.7 | (1.2) | 11.9 | (2.2) | 10.6 | (2.0) |
| ²R factor % | 24.4 | | 23.9 | | 27.8 | |
| ³R_{free} (%) | 27.4 | | 30.3 | | 34.4 | |
| ⁴RMSD bond lengths (Å) | 0.0083 | | 0.005 | | 0.004 | |
| ⁴RMSD bond angles (°) | 1.90 | | 1.08 | | 0.83 | |

Generation and Analysis of Alternative 3A4 Structure.

A second, unique, crystal form of CYP3A4 has been obtained. The spacegroup of this form was found to be P2₁2₁2. The unit cell dimensions were determined to be, to one decimal place, 88.4 Å, 110.7 Å, 113.4 Å, 90°, 90°, 90°. There are two copies in the asymmetric unit. There is no crystallographic relationship between this form and the first crystal form; analysis of the crystal packing of the two crystal forms reveals that the contacts are very different. This second crystal form diffracts to 2.8-3.0 Å.

While the morphology of the first crystal form is rod-like, the second crystal form takes on a more plate-like morphology. Crystals of both crystal forms were obtained using crystallisation conditions 0.1 M HEPES pH 7.5, 0.20-0.30 M KCl, 10-14% PEG 4000, 5% MPD, 25 mM Calcium chloride.

In addition, a second set of crystallisation conditions were also found to form the second crystal form preferentially. These conditions were 0.1 Tris-Acetic acid pH 7.5, 0.9M Sodium Formate, 10.5-12.5% MPEG 2000 or 0.1 Tris-Acetic acid pH 8.5, 0.8M Sodium Formate, 17.5% MPEG 2000.

In this crystal form, there are two copies of 3A4 in an asymmetric unit. The mathematical transformations below, detail the “co-ordinate transformation” (or “co-ordinate conversion”) from the co-ordinates of an I222 space group crystal to the co-ordinates from a P2₁2₁2 space group crystal. The atomic co-ordinates for molecules A and B in the P2₁2₁2 crystal form can be generated from those in the I222 form by the transformations:

$$\begin{matrix}\begin{matrix}\begin{matrix}and \text{:} \begin{matrix}{x}_{A} ( P21212 ) = {R}_{A} x ( 1222 ) + {t}_{A} \\ {x}_{B} ( P21212 ) = {R}_{B} x ( 1222 ) + {t}_{B}\end{matrix} \\ where \text{:}\end{matrix} \\ {R}_{A} = \begin{matrix}( - 0.9764 & - 0.1121 & - 0.1847 ) \\ ( - 0.0904 & - 0.5644 & 0.8205 ) \\ ( - 0.1962 & 0.8179 & 0.5409 )\end{matrix} {t}_{A} = \begin{matrix}( 86.361 ) \\ ( 64.676 ) \\ ( - 38.936 )\end{matrix}\end{matrix} \\ {R}_{B} = \begin{matrix}( 0.9689 & 0.2442 & - 0.0400 ) \\ ( - 0.1706 & 0.5417 & - 0.8231 ) \\ ( - 0.1793 & 0.8043 & 0.5665 )\end{matrix} {t}_{B} = \begin{matrix}( - 54.829 ) \\ ( 13.549 ) \\ ( 17.123 )\end{matrix}\end{matrix}$$

Accordingly, in another aspect the invention provides a set of atomic coordinates for a 3A4 protein which coordinates are of the A or B subunit of the P2₁2₁2 crystal form and which are obtained by applying the above mathematical transformation to the coordinate data set of Tables 1-3.

A set of coordinates in accordance with this aspect of the invention was obtained as follows:

Crystals of CYP3A4 were obtained using the crystallisation conditions 0.1 M Hepes-NaOH pH 7.5, 0.25M Potassium Chloride, 2.0% MPD, 7.0% PEG 4000, 50 mM Calcium chloride, 2.5 mM simvastatin, 2.5% ethanol. X-ray data were collected on ESRF beamline 14.1 and processed to 2.8 Å resolution using mosflm and the CCP4 suite of programs (statistics given in Table 9). Molecular replacement was performed using the program AMORE and using the CYP3A4 crystal structure described in Table 1 as the search model. The rotation function indicated two copies in the asymmetric unit, giving a solvent content of approximately 50%. After rigid body minimisation using AMORE the R factor was 35.8% and the correlation coefficient 70.2%. This molecular replacement model was then used as a starting model for subsequent refinement of other datasets from crystal form 2. The molecular replacement solution was further refined using the program Refmac to give the structure described in Table 4 with statistics described in Table 9. Inspection of the electron density maps revealed that the compound had not bound to the protein. Residues 261 to 270 were omitted from the final model due to the diffuse electron density for these regions.

TABLE 9
Data and model statistics for crystal form 2.

| Resolution | 40-2.8 Å (2.95-2.8 Å) |
|---|---|
| R merge | 7.9 (38.5) |
| I/Sigma I | 7.7 (2.0) |
| Completeness | 97.3% (94.9%) |
| Model | 2 copies of 3A4 |
| R factor | 25.7% |
| Free R factor | 32.6% % |

All publications and patents mentioned in the above specification are herein incorporated by reference. Various modifications and variations of the described invention will be apparent to those of skill in the art without departing from the scope and spirit of the invention. Although the invention has been described in connection with specific preferred embodiments, it should be understood that the invention as claimed should not be unduly limited to such specific embodiments.

1. A computer-based method for the analysis of the interaction of a molecular structure with a P450 structure, which comprises:
providing a structure comprising a three-dimensional representation of P450 3A4 or a portion of P450 3A4, which representation comprises all or a portion of the coordinates of any one of Tables 1-4±a root mean square deviation from the Cα atoms of not more than 1.5 Å;
providing a molecular structure to be fitted to said P450 3A4 structure or selected coordinates thereof; and
fitting the molecular structure to said P450 3A4 structure.

2. The method of claim 1 wherein said selected coordinates include atoms from one or more of the residues of Phe57, Phe108, Phe213, Phe215, Phe219, Phe220, Phe241 and Phe304.

3. The method of claim 1 wherein said selected coordinates include atoms from one or more of the residues identified in Table 6.

4. The method of claim 1 wherein said selected coordinates include atoms from one or more of the residues identified in Table 7.

5. The method of claim 1 which further comprises the steps of:
obtaining or synthesising a compound which has said molecular structure; and
contacting said compound with P450 protein to determine the ability of said compound to interact with the P450.

6. The method of claim 1 which further comprises the steps of:
obtaining or synthesising a compound which has said molecular structure;
forming a complex of a 3A4 P450 protein and said compound; and
analysing said complex by X-ray crystallography to determine the ability of said compound to interact with the P450.

7. The method of claim 1 which further comprises the steps of:
obtaining or synthesising a compound which has said molecular structure; and
determining or predicting how said compound is metabolised by said P450 structure; and
modifying the compound structure so as to alter the interaction between it and the P450.

8. A compound having the modified structure identified using the method of claim 7.

9. A method of obtaining a structure of a target P450 protein of unknown structure, the method comprises the steps of:
providing a crystal of said target P450;
obtaining an X-ray diffraction pattern of said crystal,
calculating a three-dimensional atomic coordinate structure of said target, by modelling the structure of said target P450 of unknown structure on the 3A4 P450 structure of any one of Tables 1-4±a root mean square deviation from the Cα atoms of not more than 1.5 Å or selected coordinates thereof.

10. The method of claim 9 wherein said target P450 protein is selected from the group consisting of 3A5, 3A7 and 3A43.

11. The method of claim 1 wherein said representation further comprises all or a portion of the coordinates of Table 5.

12. The method of claim 1 wherein the molecular structure to be fitted is in the form of a model of a pharmacophore.

13. The method of claim 1 wherein the three-dimensional representation is a model constructed from all or a portion of the coordinates of any one of Tables 1-4±a root mean square deviation from the Cα atoms of less than 0.5 Å.

14. The method of claim 13 wherein the model is: (a) a wire-frame model; (b) a chicken-wire model; (c) a ball-and-stick model; (d) a space-filling model; (e) a stick-model; (e a ribbon model; (g) a snake model; (h) an arrow and cylinder model; (i) an electron density map; (j) a molecular surface model.

15. A computer-based method for the analysis of molecular structures which comprises:
(a) providing the coordinates of at least two atoms of a P450 3A4 structure as defined in any one of Tables 1-4±a root mean square deviation from the Cα atoms of less than 1.5 Å (“selected coordinates”);
(b) providing the structure of a molecular structure to be fitted to the selected coordinates; and
(c) fitting the structure to the selected coordinates of the P450 3A4 structure.

16. The method of claim 15 wherein the selected coordinates are of at least 5, 10, 50, 100, 500 or 1000 atoms.

17. The method of claim 15 wherein the coordinates of any one of Tables 1-4 represent at least a portion of a binding pocket.

18. The method of claim 15 wherein the coordinates of any one of Tables 1-4 comprise at least 2 atoms of the amino acid residues of Table 6.

19. The method of claim 18 wherein the coordinates of any one of Tables 1-4 comprise at least 2 atoms of the amino acid residues of Table 7.

20. A computer-based method of rational drug design comprising:
(a) providing the coordinates of at least two atoms of a P450 3A4 structure as defined in any one of Tables 1-4±a root mean square deviation from the Cα atoms of less than 1.5 Å (“selected coordinates”);
(b) providing the structures of a plurality of molecular fragments;
(c) fitting the structure of each of the molecular fragments to the selected coordinates; and
(d) assembling the molecular fragments into a single molecule to form a candidate modulator molecule.

21. The method of claim 20 further comprising the step of:
(a) obtaining or synthesising the molecular fragment or modulator molecule; and
(b) contacting the molecular fragment or modulator molecule with P450 3A4 to determine the ability of the molecular fragment or modulator molecule to interact with P450 3A4.

22. A method for identifying a candidate modulator of P450 3A4 comprising the steps of: (a) employing a three-dimensional structure of P450 3A4, at least one sub-domain thereof, or a plurality of atoms thereof, to characterise at least one P450 3A4 binding cavity, the three-dimensional structure being defined by atomic coordinate data according to any one of Tables 1-4±a root mean square deviation from the Cα atoms of less than 1.5 Å; and (b) identifying the candidate modulator by designing or selecting a compound for interaction with the binding cavity.

23. The method of claim 22 further comprising the step of:
(a) obtaining or synthesising the candidate modulator; and
(b) contacting the candidate modulator with P450 3A4 to determine the ability of the candidate modulator to interact with P450 3A4.

24. A method for determining the structure of a protein, which method comprises;
providing the co-ordinates of any one of Tables 1-4±a root mean square deviation from the Cα atoms of not more than 1.5 Å or selected coordinates thereof, and
either (a) positioning said co-ordinates in the crystal unit cell of said protein so as to provide a structure for said protein, or (b) assigning NMR spectra peaks of said protein by manipulating said co-ordinates.

25. A method for determining the structure of a compound bound to P450 protein, said method comprising:
providing a crystal of P450 protein;
soaking the crystal with the compound to form a complex; and
determining the structure of the complex by employing the data of any one of Tables 1-4± a root mean square deviation from the Cα atoms of not more than 1.5 Å or a portion thereof.

26. A method for determining the structure of a compound bound to P450 protein, said method comprising:
mixing P450 protein with the compound;
crystallizing a P450 protein-compound complex; and
determining the structure of the complex by employing the data of any one of Tables 1-4±a root mean square deviation from the Cα atoms of not more than 1.5 Å or a portion thereof.

27. A method for modifying the structure of a compound in order to alter its metabolism by a P450, which method comprises:
fitting a starting compound to one or more coordinates of at least one amino acid residue of the ligand-binding region of the P450;
modifying the starting compound structure so as to increase or decrease its interaction with the ligand-binding region.

28. The method of claim 27 wherein said ligand-binding region includes at least one of the P450 residues numbered as Phe57, Phe108, Phe213, Phe215, Phe219, Phe220, Phe241 and Phe304.

29. The method of claim 28 wherein said ligand binding region includes at least 4 of said residues.

30. A method for modifying the structure of a compound in order to alter its metabolism by a P450 3A4, which method comprises:
fitting a starting compound to one or more coordinates of at least one amino acid residue of the haem-binding region of the P450;
modifying the starting compound structure so as to increase or decrease its interaction with the haem-binding region.

31. A method for modifying the structure of a compound in order to alter its, or another compounds, metabolism by a P450, which method comprises:
fitting a starting compound to one or more coordinates of at least one amino acid residue of the peripheral binding region of the P450;
modifying the starting compound structure so as to increase or decrease its interaction with the peripheral binding region;
wherein said peripheral binding region is defined as the P450 residues numbered as: 213, 214, 219.

32. A method for designing the structure of a compound which binds to the peripheral binding region, in order to alter another compounds metabolism by a P450, which method comprises:
fitting a starting compound to one or more coordinates of at least one amino acid residue of the peripheral binding region of the P450;
modifying the starting compound structure so as to increase or decrease its interaction with the peripheral binding region;
wherein said peripheral binding region is defined as the P450 residues numbered as: 213, 214, 219.

33. The method of claim 31 or 32 which further comprises fitting a second compound to the ligand binding site of said P450.

34. A method of obtaining a representation of the three dimensional structure of a crystal of cytochrome P450 3A4, which method comprises providing the data of any one of Tables 1-4 or selected coordinates thereof, and constructing a three-dimensional structure representing said coordinates.

35. A computer system, intended to generate structures and/or perform optimisation of compounds which interact with P450, P450 homologues or analogues, complexes of P450 with compounds, or complexes of P450 homologues or analogues with compounds, the system containing computer-readable data comprising one or more of:
(a) 3A4 co-ordinate data of any one of Tables 1-4, said data defining the three-dimensional structure of P450 or at least selected coordinates thereof;
(b) atomic coordinate data of a target P450 protein generated by homology modelling of the target based on the coordinate data of any one of Tables 1-4;
(c) atomic coordinate data of a target P450 protein generated by interpreting X-ray crystallographic data or NMR data by reference to the co-ordinate data of any one of Tables 1-4;
(d) structure factor data derivable from the atomic coordinate data of (b) or (c). and
(e) atomic coordinate data of any one of Tables 1-4±a root mean square deviation from the Cα atoms of not more than 1.5 Å or selected coordinates thereof.

36. A computer system according to claim 35, wherein said atomic coordinate data is for at least one of the atoms provided by the residues Phe57, Phe108, Phe213, Phe215, Phe219, Phe220, Phe241 and Phe304.

37. A computer system according to claim 35, wherein said atomic coordinate data is for at least one of the atoms provided by the residues of Table 6.

38. A computer system according to claim 37, wherein said atomic coordinate data is for at least one of the atoms provided by the residues of Table 7.

39. A computer system according to claim 35 comprising:
(i) a computer-readable data storage medium comprising data storage material encoded with said computer-readable data;
(ii) a working memory for storing instructions for processing said computer-readable data; and
(iii) a central-processing unit coupled to said working memory and to said computer-readable data storage medium for processing said computer-readable data and thereby generating structures and/or performing rational drug design.

40. A computer system according to claim 39 further comprising a display coupled to said central-processing unit for displaying said structures.

41. A method of providing data for generating structures and/or performing optimisation of compounds which interact with P450, P450 homologues or analogues, complexes of P450 with compounds, or complexes of P450 homologues or analogues with compounds, the method comprising:
(i) establishing communication with a remote device containing
(a) computer-readable data comprising atomic coordinate data of any one of Tables 1-4±a root mean square deviation from the Cα atoms of not more than 1.5 Å or selected coordinates thereof;
(b) atomic coordinate data of a target P450 homologue or analogue generated by homology modelling of the target based on the data (a);
(c) atomic coordinate data of a protein generated by interpreting X-ray crystallographic data or NMR data by reference to the data of any one of Tables 1-4 and
(d) structure factor data derivable from the atomic coordinate data of (d) or (e); and
(ii) receiving said computer-readable data from said remote device.

42. The method of claim 41 wherein said atomic coordinate data is that of any one of Tables 1-4±a root mean square deviation from the Cα atoms of not more than 1.5 Å or a selected portion thereof.

43. A computer-readable storage medium, comprising a data storage material encoded with computer readable data, wherein the data are defined by all or a portion of the structure coordinates of the P450 protein of any one of Tables 1-4 or a homologue of P450, wherein said homologue comprises backbone atoms that have a root mean square deviation from the backbone atoms of said any one of Tables 1-4 respectively of not more than 1.5 Å.

44. A computer-readable storage medium comprising a data storage material encoded with a first set of computer-readable data comprising a Fourier transform of at least a portion of the structural coordinates for the P450 protein defined by the structure of any one of Tables 1-4±a root mean square deviation from the Cα atoms of not more than 1.5 Å or selected coordinates thereof; which data, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.

45. A crystal of P450 3A4.

46. The crystal of claim 45 in apo form.

47. A co-crystal of P450 3A4 and a ligand.

48. A crystal of P450 3A4 having an orthorhomobic space group I222.

49. The crystal of claim 47 having unit cell dimensions 78 Å, 100 Å, 132 Å, 90°, 90°, 90° with a unit cell variability of 5% in all dimensions.

50. A crystal of P450 3A4 having a space group space group P2₁2₁2.

51. The crystal of claim 50 with cell dimensions of 88 Å, 111 Å, 113 Å, 90°, 90°, 90° with a unit cell variability of 5% in all dimensions.

52. The crystal of claim 45 or 47 wherein said 3A4 comprises the sequence of SEQ ID NO:2

53. A crystal of P450 3A4 protein having a resolution better than 3.1 Å.

54. A crystal of P450 protein having the structure defined by the co-ordinates of any one of Tables 1-4±a root mean square deviation from the Cα atoms of not more than 1.5 Å.

55. A method of predicting three dimensional structures of P450 homologues or analogues of unknown structure, the method comprises the steps of:
aligning a representation of an amino acid sequence of a target P450 protein of unknown three-dimensional structure with the amino acid sequence of the P450 of any one of Tables 1-4±a root mean square deviation from the Cα atoms of not more than 1.5 Å to match homologous regions of the amino acid sequences;
modelling the structure of the matched homologous regions of said target P450 of unknown structure on the corresponding regions of the P450 structure as defined by said any one of Tables 1-4 respectively±a root mean square deviation from the Cα atoms of not more than 1.5 Å; and
determining a conformation for said target P450 of unknown structure which substantially preserves the structure of said matched homologous regions.

56. The method of claim 55 wherein said target P450 protein is selected from the group consisting of 3A5, 3A7 or 3A43.

57. A chimaeric protein having a binding cavity which provides a substrate specificity substantially identical to that of P450 3A4 protein,
wherein the chimaeric protein binding cavity is lined by a plurality of atoms which correspond to selected P450 3A4 atoms lining the P450 3A4 binding cavity, the relative positions of said plurality of atoms corresponding to the relative positions, as defined by any one of Tables 1-4±a root mean square deviation from the Cα atoms of not more than 1.5 Å, of said selected P450 3A4 atoms.

58. A method of assessing the ability of a compound to interact with P450 3A4 protein which comprises:
obtaining or synthesising said compound;
forming a crystallised complex of a P450 3A4 protein and said compound, said complex diffracting X-rays for the determination of atomic coordinates of said complex to a resolution of better than 2.8 Å; and
analysing said complex by X-ray crystallography to determine the ability of said compound to interact with the P450 3A4 protein.

59. A method of preparing a composition comprising identifying a molecular structure or modulator according to the method of claim 40 or 42, and admixing the molecule with a carrier.

60. A process for producing a medicament, pharmaceutical composition or drug, the process comprising: (a) identifying a molecular structure or modulator according to the method as defined in claim 20 or 22; and (b) preparing a medicament, pharmaceutical composition or drug containing the optimised modulator molecule.

61. A process according to claim 60 which comprises (a) identifying a molecular structure or modulator according to the method as defined in any one of claims 28 to 40; (b) optimising the structure of the modulator molecule; and (c) preparing a medicament, pharmaceutical composition or drug containing the optimised modulator molecule.

62. A compound identified, produced or obtainable by the process or method of claim 20 or 22.

63. A compound of claim 62 or composition thereof for use in medicine.

64. A computer-based method for identifying a candidate modulator of P450 3A4 comprising the steps of:
employing a three-dimensional structure of P450 3A4, or selected co-ordinates thereof, the three-dimensional structure being defined by atomic coordinate data according to any one of Tables 1-4±a root mean square deviation from the Cα atoms of less than 1.5 Å;
identifying the candidate modulator by designing or selecting a compound for interaction with the binding cavity.

65. The method of claim 27 wherein said ligand-bindingregion includes at least one of the P450 residues of Table 6 or Table 7.

66. The method of claim 65 wherein said ligandbinding region includes at least 4 of said residues.
